(19) United States
(12) Patent Application Publication
(10) Pub. No.: US 2013/0109682 A1
(43) Pub. Date: May 2, 2013

(54) CYCLIC ETHER COMPOUNDS USEFUL AS KINASE INHIBITORS

(71) Applicants: Matthew Burger, Albany, CA (US); Yu Ding, Union City, CA (US); Wooseok Han, San Ramon, CA (US); Mika Lindvall, Oakland, CA (US); Gisele A. Nishiguchi, Albany, CA (US); Alice Rico, Castro Valley, CA (US); Aaron Smith, Fremont, CA (US); Huw Tanner, Alameda, CA (US); Lifeng Wan, Union City, CA (US)

(72) Inventors: Matthew Burger, Albany, CA (US); Yu Ding, Union City, CA (US); Wooseok Han, San Ramon, CA (US); Mika Lindvall, Oakland, CA (US); Gisele A. Nishiguchi, Albany, CA (US); Alice Rico, Castro Valley, CA (US); Aaron Smith, Fremont, CA (US); Huw Tanner, Alameda, CA (US); Lifeng Wan, Union City, CA (US)

(73) Assignee: NOVARTIS AG, Basel (CH)

(21) Appl. No.: 13/807,993

(22) PCT Filed: Jul. 4, 2011
(86) PCT No.: PCT/EP11/61198

§ 371 Date: Jan. 2, 2013

Related U.S. Application Data
(60) Provisional application No. 61/361,755, filed on Jul. 6, 2010.

(51) Int. Cl. C07D 417/14; A61K 31/444; A61K 45/06; A61K 31/506; A61K 31/501; A61K 31/541; C07D 405/14; A61K 31/497

(52) U.S. Cl. CPC C07D 417/14; C07D 405/14; A61K 31/444; A61K 31/497; A61K 31/506; A61K 31/501; A61K 31/541; A61K 45/06
USPC 514/227.8; 546/256; 514/333; 544/405; 514/255.05; 544/322; 514/256; 544/238; 514/252.03; 544/58.2

(57) ABSTRACT

The present invention provides certain compounds of Formula (I):

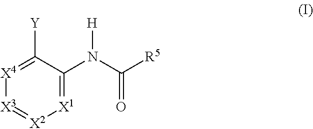

and pharmaceutically acceptable salts thereof, as further described herein. Also provided are formulations comprising compounds of formula I, and a method to use such compounds for treating a disease or condition mediated by Provirus Integration of Maloney Kinase (PIM Kinase), GSK3, PKC, KDR, PDGFRa, FGFR3, FLT3, or cABL.

### FIELD OF THE INVENTION

The present invention relates to new compounds that are inhibitors of protein kinases, and the new compounds tautomers and stereoisomers, and pharmaceutically acceptable salts, esters, metabolites or prodrugs thereof, and compositions of the new compounds together with pharmaceutically acceptable carriers. The present invention also relates to uses of the new compounds, either alone or in combination with at least one additional therapeutic agent, in the prophylaxis or treatment of various disorders, including cancer.

### BACKGROUND

Protein kinases constitute a large family of structurally related enzymes that are responsible for the control of a variety of signal transduction processes within the cell. (See, Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serinelthreonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂0₂), cytokines (e.g., interleukin-1 (L-1) and tumor necrosis factor a (TNF-α, cytokines (e.g., interleukin-1 (L-1) to macrophagecolony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there has been a substantial effort in medicinal chemistry to find protein kinase inhibitors that are effective as therapeutic agents.

Glycogen synthase kinase 3 (GSK3) is a serine/threonine kinase for which two isoforms, α and β, have been identified. Woodgett, Trends Biochem. Sci., 16:177-81 (1991). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such glucose transport. Subsequently, it has been shown that GSK3 activity is also inactivated by other growth factors that, like insulin, signal through receptor tyrosine kinases (RTKs). Examples of such signaling molecules include IGF-1 and EGF. Saito et al., Biochem. J., 303:27-31 (1994); Welsh et al., Biochem. J. 294:625-29 (1993); and Cross et al., Biochem. J., 303:21-26 (1994).

Agents that inhibit GSK3 activity are useful in the treatment of disorders that are mediated by GSK3 activity. In addition, inhibition of GSK3 mimics the activation of growth factor signaling pathways and consequently GSK3 inhibitors are useful in the treatment of diseases in which such pathways are insufficiently active. Examples of diseases that can be treated with GSK3 inhibitors are described below.

Diabetes mellitus is a serious metabolic disease that is defined by the presence of chronically elevated levels of blood glucose (hyperglycemia). This state of hyperglycemia is the result of a relative or absolute lack of activity of the peptide hormone, insulin. Insulin is produced and secreted by the B cells of the pancreas. Insulin is reported to promote glucose utilization, protein synthesis, and the formation and storage of carbohydrate energy as glycogen. Glucose is stored in the body as glycogen, a form of polymerized glucose, which may be converted back into glucose to meet metabolism requirements. Under normal conditions, insulin is secreted at both a basal rate and at enhanced rates following glucose stimulation, all to maintain metabolic homeostasis by the conversion of glucose into glycogen.

The term diabetes mellitus encompasses several different hyperglycemic states. These states include Type 1 (insulin-dependent diabetes mellitus or IDDM) and Type 2 (non-insulin dependent diabetes mellitus or NIDDM) diabetes. The hyperglycemia present in individuals with Type 1 diabetes is associated with deficient, reduced, or nonexistent levels of insulin that are insufficient to maintain blood glucose levels within the physiological range. Conventionally, Type 1 diabetes is treated by administration of replacement doses of insulin, generally by a parental route. Since GSK3 inhibition stimulates insulin-dependent processes, it is consequently useful in the treatment of type 1 diabetes.

Type 2 diabetes is an increasingly prevalent disease of aging. It is initially characterized by decreased sensitivity to insulin and a compensatory elevation in circulating insulin concentrations, the latter of which is required to maintain normal blood glucose levels. Increased insulin levels are caused by increased secretion from the pancreatic beta cells, and the resulting hyperinsulinemia is associated with cardiovascular complications of diabetes. As insulin resistance worsens, the demand on the pancreatic beta cells steadily increases until the pancreas can no longer provide adequate levels of insulin, resulting in elevated levels of glucose in the blood. Ultimately, overt hyperglycemia and hyperlipidemia occur, leading to the devastating long-term complications associated with diabetes, including cardiovascular disease, renal failure and blindness. The exact mechanism(s) causing type 2 diabetes are unknown, but result in impaired glucose transport into skeletal muscle and increased hepatic glucose production, in addition to inadequate insulin response. Dietary modifications are often ineffective, therefore the majority of patients ultimately require pharmaceutical intervention in an effort to prevent and/or slow the progression of the complications of the disease. Many patients can be treated with one or more of the many oral anti-diabetic agents available, including sulfonylureas, to increase insulin secretion. Examples of sulfonylurea drugs include metformin for suppression of hepatic glucose production, and troglitazone, an insulin-sensitizing medication. Despite the utility of these agents, 30-40% of diabetics are not adequately controlled using these medications and require subcutaneous insulin injections. Additionally, each of these therapies has associated side effects. For example, sulfonylureas can cause hypoglycemia and troglitazone can cause severe hepatoxicity. Presently, there is a need for new and improved drugs for the treatment of prediabetic and diabetic patients.

As described above, GSK3 inhibition stimulates insulin-dependent processes and is consequently useful in the treatment of type 2 diabetes. Recent data obtained using lithium salts provides evidence for this notion. The lithium ion has recently been reported to inhibit GSK3 activity. Klein et al., PNAS 93:8455-9 (1996). Since 1924, lithium has been reported to have antidiabetic effects including the ability to reduce plasma glucose levels, increase glycogen uptake, potentiate insulin, up-regulate glucose synthase activity and to stimulate glycogen synthesis in skin, muscle and fat cells. However, lithium has not been widely accepted for use in the inhibition of GSK3 activity, possibly because of its documented effects on molecular targets other than GSK3. The purine analog 5-iodotubercidin, also a GSK3 inhibitor, likewise stimulates glycogen synthesis and antagonizes inactivation of glycogen synthase by glucagon and vasopressin in rat liver cells. Fluckiger-Isler et al., Biochem J 292:85-91 (1993); and Massillon et al., Biochem J 299:123-8 (1994). However, this compound has also been shown to inhibit other serine/threonine and tyrosine kinases. Massillon et al., Biochem J 299:123-8 (1994).

One of the main goals in the management of patients with diabetes mellitus is to achieve blood glucose levels as close to normal as possible. In general, obtaining normal postprandial blood glucose levels is more difficult than normalizing fasting hyperglycemia. In addition, some epidemiological studies suggest that postprandial hyperglycemia (PPHG) or hyperinsulinemia are independent risk factors for the development of macrovascular complications of diabetes mellitus. Recently, several drugs with differing pharmacodynamic profiles have been developed which target PPHG. These include insulin lispro, amylin analogues, alpha-glucosidase inhibitors and meglitinide analogues. Insulin lispro has a more rapid onset of action and shorter duration of efficacy compared with regular human insulin. In clinical trials, the use of insulin lispro has been associated with improved control of PPHG and a reduced incidence of hypoglycemic episodes. Repaglinide, a meglitinide analogue, is a short-acting insulinotropic agent which, when given before meals, stimulates endogenous insulin secretions and lowers postprandial hyperglycemic excursions. Both insulin lispro and repaglinide are associated with postprandial hyperinsulinemia. In contrast, amylin analogues reduce PPHG by slowing gastric emptying and delivery of nutrients to the absorbing surface of the gut. Alpha-glucosidase inhibitors such as acarbose, miglitol and voglibose also reduce PPHG primarily by interfering with the carbohydrate-digesting enzymes and delaying glucose absorption. Yamasaki et al., Tohoku J Exp Med 1997 November; 183(3):173-83. The GSK inhibitors of the present invention are also useful, alone or in combination with the agents set forth above, in the treatment of postprandial hyperglycemia as well as in the treatment of fasting hyperglycemia.

GSK3 is also involved in biological pathways relating to Alzheimer's disease (AD). The characteristic pathological features of AD are extracellular plaques of an abnormally processed form of the amyloid precursor protein (APP), so called β3-amyloid peptide (β-AP) and the development of intracellular neurofibrillary tangles containing paired helical filaments (PHF) that consist largely of hyperphosphorylated tau protein. GSK3 is one of a number of kinases that have been found to phosphorylate tau protein in vitro on the abnormal sites characteristic of PHF tau, and is the only kinase also demonstrated to do this in living cells and in animals. Lovestone et al., Current Biology 4:1077-86 (1994); and Brownlees et al., Neuroreport 8: 3251-3255 (1997). Furthermore, the GSK3 kinase inhibitor, LiCl, blocks tau hyperphosphorylation in cells. Stambolic et al., Current Biology 6:1664-8 (1996). Thus GSK3 activity may contribute to the generation of neurofibrillary tangles and consequently to disease progression. Recently it has been shown that GSK3β associates with another key protein in AD pathogenesis, presenillin 1 (PS1). Takashima et al., PNAS 95:9637-9641 (1998). Mutations in the PS1 gene lead to increased production of β-AP, but the authors also demonstrate that the mutant PS1 proteins bind more tightly to GSK3β and potentiate the phosphorylation of tau, which is bound to the same region of PS1.

Interestingly it has also been shown that another GSK3 substrate, β-catenin, binds to PS1. Zhong et al., Nature 395:698-702 (1998). Cytosolic β-catenin is targeted for degradation upon phosphorylation by GSK3 and reduced β-catenin activity is associated with increased sensitivity of neuronal cells to β-AP induced neuronal apoptosis. Consequently, increased association of GSK3β with mutant PS1 may account for the reduced levels of β-catenin that have been observed in the brains of PS1-mutant AD patients and to the disease related increase in neuronal cell-death. Consistent with these observations, it has been shown that injection of GSK3 antisense but not sense, blocks the pathological effects of β-AP on neurons in vitro, resulting in a 24 hr delay in the onset of cell death. Takashima et al., PNAS 90:7789-93. (1993). In these latter studies, the effects on cell-death are preceded (within 3-6 hours of β-AP administration) by a doubling of intracellular GSK3 activity, suggesting that in addition to genetic mechanisms may increase GSK3 activity. Further evidence for a role for GSK3 in AD is provided by the observation that the protein expression level (but, in this case, not specific activity) of GSK3 is increased by 50% in postsynaptosomal supernatants of AD vs. normal brain tissue. Pei et al., J Neuropathol Exp 56:70-78 (1997).

Even more recently, it has been shown that therapeutic concentrations of lithium, a known GSK3 inhibitor, block the production of β-AP by interfering with amyloid precursor protein (APP) cleavage. Phiel et al., Nature 423(22): 435-438 (2003). Since GSK3 also phosphorylates tau protein, the principal component of neurofibrillary tangles, inhibition of GSK3 provides both a reduction in amyloid plaques and neurofibrillary tangles, and is useful in the treatment of Alzheimer's disease.

In addition to the effects of lithium described above, there is a long history of the use of lithium to treat bipolar disorder (manic depressive syndrome). This clinical response to lithium may reflect an involvement of GSK3 activity in the etiology of bipolar disorder, in which case GSK3 inhibitors could be relevant to that indication. In support of this notion it was recently shown that valproate, another drug commonly used in the treatment of bipolar disorder, is also a GSK3 inhibitor. Chen et al., J. Neurochemistry 72:1327-1330 (1999). One mechanism by which lithium and other GSK3 inhibitors may act to treat bipolar disorder is to increase the survival of neurons subjected to aberrantly high levels of excitation induced by the neurotransmitter, glutamate. Nonaka et al., PNAS 95: 2642-2647 (1998). Glutamate-induced neuronal excitotoxicity is also believed to be a major cause of neurodegeneration associated with acute damage, such as in cerebral ischemia, traumatic brain injury and bacterial infection. Furthermore it is believed that excessive glutamate signaling is a factor in the chronic neuronal damage seen in diseases such as Alzheimer's, Huntingdon's, Parkinson's, AIDS associated dementia, amyotrophic lateral sclerosis (ALS) and multiple sclerosis (MS). Thomas, J. Am. Geriatr. Soc. 43: 1279-89 (1995). Consequently GSK3 inhibitors are believed to be a useful treatment in these and other neurodegenerative disorders.

GSK3 phosphorylates transcription factor NF-AT and promotes its export from the nucleus, in opposition to the effect of calcineurin. Beals et al., Science 275:1930-33 (1997). Thus, GSK3 blocks early immune response gene activation via NF-AT, and GSK3 inhibitors may tend to permit or prolong activation of immune responses. Thus GSK3 inhibitors are believed to prolong and potentiate the immunostimulatory effects of certain cytokines, and such an effect may enhance the potential of those cytokines for tumor immunotherapy or indeed for immunotherapy in general.

Lithium also has other biological effects. It is a potent stimulator of hematopoiesis, both in vitro and in vivo. Hammond et al., Blood 55: 26-28 (1980). In dogs, lithium carbonate eliminated recurrent neutropenia and normalized other blood cell counts. Doukas et al. Exp Hematol 14: 215-221 (1986). If these effects of lithium are mediated through the inhibition of GSK3, GSK3 inhibitors may have even broader applications.

Infection with the Maloney retrovirus and genome integration in the host cell genome results in development of lymphomas in mice. Provirus Integration of Maloney Kinase (PIM-Kinase) was identified as one of the frequent proto-oncogenes capable of being transcriptionally activated by this retrovirus integration event (Cuypers H T et al., “Murine leukemia virus-induced T-cell lymphomagenesis: integration of proviruses in a distinct chromosomal region,” Cell 37(1):141-50 (1984); Selten G, et al., “Proviral activation of the putative oncogene Pim-1 in MuLV induced T-cell lymphomas” EMBO J 4(7):1793-8 (1985)), thus establishing a correlation between over-expression of this kinase and its oncogenic potential. Sequence homology analysis demonstrated that there are 3 highly homologous Pim-Kinases (Pim1, 2 & 3), Pim1 being the proto-oncogene originally identified by retrovirus integration. Furthermore, transgenic mice over-expressing Pim1 or Pim2 show increased incidence of T-cell lymphomas (Breuer M et al., “Very high frequency of lymphoma induction by a chemical carcinogen in pim-1 transgenic mice” Nature 340(6228):61-3 (1989)), while over-expression in conjunction with c-myc is associated with incidence of B-cell lymphomas (Verbeek S et al., “Mice bearing the E mu-myc and E mu-pim-1 transgenes develop pre-B-cell leukemia prenatally” Mol Cell Biol 11(2):1176-9 (1991)). Thus, these animal models establish a strong correlation between Pim over-expression and oncogenesis in hematopoietic malignancies. In addition to these animal models, Pim over-expression has been reported in many other human malignancies. Pim1, 2 & 3 over-expression is frequently observed in many hematopoietic malignancies (Amson R et al., “The human protooncogene product p33pim is expressed during fetal hematopoiesis and in diverse leukemias,” PNAS USA 86(22):8857-61 (1989); Cohen A M et al., “Increased expression of the hPim-2 gene in human chronic lymphocytic leukemia and non-Hodgkin lymphoma,” Leuk Lymph 45(5):951-5 (2004), Huttmann A et al., “Gene expression signatures separate B-cell chronic lymphocytic leukaemia prognostic subgroups defined by ZAP-70 and CD38 expression status,” Leukemia 20:1774-1782 (2006)) and in prostate cancer (Dhanasekaran S M, et al., “Delineation of prognostic biomarkers in prostate cancer,” Nature 412(6849):822-6 (2001); Cibull T L, et al., “Overexpression of Pim-1 during progression of prostatic adenocarcinoma,” J Clin Pathol 59(3):285-8 (2006)), while over-expression of Pim3 is frequently observed in hepatocellular carcinoma (Fujii C, et al., “Aberrant expression of serine/threonine kinase Pim-3 in hepatocellular carcinoma development and its role in the proliferation of human hepatoma cell lines,” Int J Cancer 114:209-218 (2005)) and pancreatic cancer (Li Y Y et al., “Pim-3, a proto-oncogene with serine/threonine kinase activity, is aberrantly expressed in human pancreatic cancer and phosphorylates bad to block bad-mediated apoptosis in human pancreatic cancer cell lines,” Cancer Res 66(13):6741-7 (2006)).

Pim1, 2 & 3 are Serine/Threonine kinases that normally function in survival and proliferation of hematopoietic cells in response to growth factors and cytokines Cytokines signaling through the Jak/Stat pathway leads to activation of transcription of the Pim genes and synthesis of the proteins. No further post-translational modifications are required for the Kinase Pim activity. Thus, signaling down stream is primarily controlled at the transcriptional/translational and protein turnover level. Substrates for Pim kinases include regulators of apoptosis such as the Bcl-2 family member BAD (Aho T et al., “Pim-1 kinase promotes inactivation of the pro-apoptotic Bad protein by phosphorylating it on the Ser112 gatekeeper site: FEBS Letters 571: 43-49 (2004)), cell cycle regulators such as p21^{WFA1/CIP1} (Wang Z, et al., “Phosphorylation of the cell cycle inhibitor p21Cip1/WAF1 by Pim-1 kinase,” Biochem Biophys Acta 1593:45-55 (2002)), CDC25A (1999), C-TAK (Bachmann M et al., “The Oncogenic Serine/Threonine Kinase Pim-1 Phosphorylates and Inhibits the Activity of Cdc25C-associated Kinase 1 (C-TAK1). A novel role for Pim-1 at the G2/M cell cycle checkpoint,” J Biol Chem 179:48319-48328 (2004)) and NuMA (Bhattacharya N, et al., “Pim-1 associates with protein complexes necessary for mitosis,” Chromosoma 111(2):80-95 (2002)) and the protein synthesis regulator 4EBP1 (Hammerman P S et al., “Pim and Akt oncogenes are independent regulators of hematopoietic cell growth and survival,” Blood 105(11):4477-83 (2005)). The effects of Pim(s) in these regulators are consistent with a role in protection from apoptosis and promotion of cell proliferation and growth. Thus, over-expression of Pim(s) in cancer is thought to play a role in promoting survival and proliferation of cancer cells and, therefore, their inhibitions should be an effective way of treating cancers on which they are over-expressed. In fact several reports indicate that knocking down expression of Pim(s) with siRNA results in inhibition of proliferation and cell death (Dai J M, et al., “Antisense oligodeoxynucleotides targeting the serine/threonine kinase Pim-2 inhibited proliferation of DU-145 cells,” Acta Pharmacol Sin 26(3):364-8 (2005); Fujii et al. 2005; Li et al. 2006). Furthermore, mutational activation of several well know oncogenes in hematopoietic malignancies are thought exert its effects at least in part through Pim(s). For example, targeted down regulation of pim expression impairs survival of hematopoietic cells transformed by Flt3 and BCR/ABL (Adam et al. 2006). Thus, inhibitors to Pim1, 2 &3 would be useful in the treatment of these malignancies.

In addition to a potential role in cancer treatment and myeloproliferative diseases, such inhibitor could be useful to control expansion of immune cells in other pathologic condition such as autoimmune diseases, allergic reactions and in organ transplantation rejection syndromes. This notion is supported by the findings that differentiation of Th1 Helper T-cells by IL-12 and IFN-α results in induction of expression of both Pim1 and Pim2 (Aho T et al., “Expression of human Pim family genes is selectively up-regulated by cytokines promoting T helper type 1, but not T helper type 2, cell differentiation,” Immunology 116: 82-88 (2005)). Moreover, Pim(s) expression is inhibited in both cell types by the immunosuppressive TGF-β (Aho et al. 2005). These results suggest that Pim kinases are involved in the early differentiation process of Helper T-cells, which coordinate the immunological responses in autoimmune diseases, allergic reaction and tissue transplant rejection. Recent reports demonstrate that Pim kinase inhibitors show activity in animal models of inflammation and autoimmune diseases. See JE Robinson “Targeting the Pim Kinase Pathway for Treatment of Autoimmune and Inflammatory Diseases,” for the Second Annual Conference on Anti-Inflammatories: Small Molecule Approaches,” San Diego, Calif. (Conf. April 2011; Abstract published earlier on-line). Accordingly, compounds that inhibit Pim kinases are predicted to be useful to treat such autoimmune disorders as Crohn's disease, inflammatory bowel disease, rheumatoid arthritis, and chronic inflammatory diseases.

A continuing need exists for compounds that inhibit the proliferation of capillaries, inhibit the growth of tumors, treat cancer, modulate cell cycle arrest, and/or inhibit molecules such as Pim1, Pim2 and Pim3, and pharmaceutical formulations and medicaments that contain such compounds. A need also exists for methods of administering such compounds, pharmaceutical formulations, and medicaments to patients or subjects in need thereof.

Capillaries reach into almost all tissues of the human body and supply tissues with oxygen and nutrients as well as removing waste products. Under typical conditions, the endothelial cells lining the capillaries do not divide, and capillaries, therefore, do not normally increase in number or size in a human adult. Under certain normal conditions, however, such as when a tissue is damaged, or during certain parts of the menstrual cycle, the capillaries begin to proliferate rapidly. This process of forming new capillaries from pre-existing blood vessels is known as angiogenesis or neovascularization. See Folkman, J. Scientific American 275, 150-154 (1996). Angiogenesis during wound healing is an example of pathophysiological neovascularization during adult life. During wound healing, the additional capillaries provide a supply of oxygen and nutrients, promote granulation tissue, and aid in waste removal. After termination of the healing process, the capillaries normally regress. Lymboussaki, A. “Vascular Endothelial Growth Factors and their Receptors in Embryos, Adults, and in Tumors” Academic Dissertation, University of Helsinki, Molecular/Cancer Biology Laboratory and Department of Pathology, Haartman Institute, (1999).

Angiogenesis also plays an important role in the growth of cancer cells. It is known that once a nest of cancer cells reaches a certain size, roughly 1 to 2 mm in diameter, the cancer cells must develop a blood supply in order for the tumor to grow larger as diffusion will not be sufficient to supply the cancer cells with enough oxygen and nutrients. Thus, inhibition of angiogenesis is expected to retard or halt the growth of cancer cells.

Receptor tyrosine kinases (RTKs) are transmembrane polypeptides that regulate developmental cell growth and differentiation and remodeling and regeneration of adult tissues. Mustonen, T. et al., J. Cell Biology 129, 895-898 (1995); van der Geer, P. et al. Ann Rev. Cell Biol. 10, 251-337 (1994). Polypeptide ligands known as growth factors, or cytokines, are known to activate RTKs. Signaling of RTKs involves ligand binding and a shift in conformation in the external domain of the receptor resulting in its dimerization. Lymboussaki, A. “Vascular Endothelial Growth Factors and their Receptors in Embryos, Adults, and in Tumors” Academic Dissertation, University of Helsinki, Molecular/Cancer Biology Laboratory and Department of Pathology, Haartman Institute, (1999); Ullrich, A. et al., Cell 61, 203-212 (1990). Binding of the ligand to the RTK results in receptor trans-phosphorylation at specific tyrosine residues and subsequent activation of the catalytic domains for the phosphorylation of cytoplasmic substrates.

Two subfamilies of RTKs are specific to the vascular endothelium. These include the vascular endothelial growth factor (VEGF) subfamily and the Tie receptor subfamily. Class III RTKs include VEGFR-1, VEGFR-2, and VEGFR-3. Shibuya, M. et al., Oncogene 5, 519-525 (1990); Terman, B. et al., Oncogene 6, 1677-1683 (1991); Aprelikova, O. et al., Cancer Res. 52, 746-748 (1992).

Members of the VEGF subfamily have been described as being able to induce vascular permeability and endothelial cell proliferation and further identified as a major inducer of angiogenesis and vasculogenesis. Ferrara, N. et al., Endocrinol. Rev. 18, 4-25 (1997). VEGF is known to specifically bind to RTKs including VEGFR-1 and VEGFR-2. DeVries, C. et al., Science 255, 989-991 (1992); Quinn, T. et al., Proc. Natl. Acad. Sci. 90, 7533-7537 (1993). VEGF stimulates the migration and proliferation of endothelial cells and induces angiogenesis both in vitro and in vivo. Connolly, D. et al., J. Biol. Chem. 264, 20017-20024 (1989); Connolly, D. et al., J. Clin. Invest. 84, 1470-1478 (1989); Ferrara, N. et al., Endocrino. Rew. 18, 4-25 (1997); Leung, D. et al., Science 246, 1306-1309 (1989); Plouet, J. et al., EMBO J. 8, 3801-3806 (1989).

Because angiogenesis is known to be critical to the growth of cancer and to be controlled by VEGF and VEGF-RTK, substantial efforts have been undertaken to develop therapeutics that are antagonists of VEGF-RTK to thereby inhibit or retard angiogenesis, and hopefully interfere or stop tumor proliferation.

Phospholipid- and calcium-dependent protein kinase C occurs in cells in a number of forms and participates in various fundamental processes, such as signal transmission, proliferation and differentiation, and also the release of hormones and neurotransmitters. The activation of that enzyme is effected either by receptor-mediated hydrolysis of phospholipids of the cell membrane or by direct interaction with certain turnout-promoting active substances. The sensitivity of the cell to receptor-mediated signal transmission can be substantially influenced by modifying the activity of protein kinase C (as a signal transmitter). Compounds that are capable of influencing the activity of protein kinase C can be used as tumour-inhibiting, as antiinflammatory, immunomodulating and antibacterial active ingredients and may even be of value as agents against atherosclerosis and disorders of the cardiovascular system and central nervous system.

The Philadelphia Chromosome is a hallmark for chronic myelogenous leukaemia (CML) and carries a hybrid gene that contains N-terminal exons of the BCR gene and the major C terminal part (exons 2-1 l) of the ABL gene. This gene encodes a 210 kD protein, p210 Bcr-Abl, the Abl sequence of which contains the Abl tyrosine kinase domain which is tightly regulated in the wild type c-Abl, but constitutively activated in the Bcr-Abl fusion protein. This deregulated tyrosine kinase interacts with multiple cellular signaling pathways leading to transformation and deregulated proliferation of the cells (Lugo et al., Science 247, 1079, 1990). Mutant forms of the Bcr-Abl protein have also been identified. A detailed review of Bcr-Abl mutant forms has been published (Cowan-Jones et a/, Mini Reviews in Medicinal Chemistry, 2004, 4 285-299). Compounds that are capable of influencing the activity of Abl, especially mutant forms can be used as tumor-inhibiting agents.

### SUMMARY OF INVENTION

The present invention provides compounds of Formula I, their stereoisomers, tautomers and pharmaceutically acceptable salts thereof:

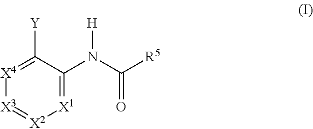

wherein,
X¹ represents CR¹ or N;
X² represents CR² or N;
X³ represents CR³ or N;
X⁴ represents CR⁴ or N;
   provided that not more than two of X¹, X², X³, and X⁴ can be N;
Y is selected from a group consisting of heterocyclo-alkyl, and partially unsaturated heterocyclo-alkyl, wherein each said Y group is independently substituted with at least one of R⁷, R⁸, R⁹, R¹⁰, R¹¹, R¹², R¹³, R¹⁴, and R¹⁵;
R¹, R², R³, and R⁴ independently are selected from the group consisting of hydrogen, halo, hydroxyl, nitro, cyano, SO₃H and substituted or unsubstituted alkyl, alkenyl, alkynyl, alkoxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, alkylthio, aryl, heteroaryl, cycloalkyl, hetero cycloalkyl, partially saturated cycloalkyl, aryloxy, heteroaryloxy, heterocyclyloxy, cycloalkyloxy, acyl, acylamino and acyloxy;
R⁵ is selected from a group consisting of thiazole, pyridine, pyrazole, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from hydroxy, hydroxy-C₁₋₄-alkyl, C₁₋₄-alkyl, H, D, C₁₋₄-halo-alkyl, C₁₋₄ alkoxy, —(CH₂)₁₋₄—X (where X is amino, C₁₋₄ alkoxy, hydroxy, F, Cl), amino, C₃₋₆-cycloalkyl, C₃₋₆ heterocyclo-alkyl, C₂₋₄ alkynyl, C₂₋₄ alkylene, (CH₂)₁₋₄—CN, (CH₂)₁₋₄—CONH₂, (CH₂)₁₋₄—CO₂H, carboxy, cyano, oxo, CONR₂ (where each R is independently H or C₁₋₄ alkyl), and halogen; alternatively any two of R¹¹, R¹², R¹³, R¹⁴, and R¹⁵ along with the carbon atom or atoms that they are attached to can form a C₃₋₈-cycloalkyl or a C₃₋₈-heterocycloalkyl group that can be substituted with up to two groups selected from hydroxy, hydroxy-C₁₋₄-alkyl, C₁₋₄-alkyl, C₁₋₄-halo-alkyl, C₁₋₄ alkoxy, —(CH₂)₁₋₄—X (where X is amino, C₁₋₄ alkoxy, hydroxy, F, Cl), amino, C₂₋₄ alkynyl, C₂₋₄ alkylene, (CH₂)₁₋₄—CN, (CH₂)₁₋₄—CONH₂, (CH₂)₁₋₄—CO₂H, carboxy, cyano, oxo, CONR₂ (where each R is independently H or C₁₋₄ alkyl), and halogen; or two of R¹¹, R¹², R¹³, R¹⁴, and R¹⁵ when attached to the same carbon can form an exocyclic methylene (═CH₂);
R¹⁸, R¹⁹, and R²⁰ independently are selected from H, aryl, heteroaryl, hydroxy, amino, cyano, halogen, and C₁₋₆-alkyl, C₃₋₈-cycloalkyl, C₃₋₈-heterocycloalkyl, wherein said aryl, alkyl, heteroaryl, alkyl, cycloalkyl and heterocycloalkyl groups are further substituted with at least one of R²¹, R²², or R²³; and
R²¹, R²², and R²³ independently are selected from halogen, D, C₁₋₄-alkyl, amino, —NHC(O)—C₁₋₄ alkyl, COOH, hydroxy, oxo, CN, NO₂, H, CONH—C₁₋₄ alkyl, CO—NH—C₃₋₆-branched alkyl, —OC₁₋₄-alkyl, —SO₂—C₁₋₄ alkyl, —(CH₂)₁₋₄—X where X is OH, OMe, CN, or halo, and —OC₁₋₄-haloalkyl.

These compounds inhibit one or more of the kinases discussed above, especially one or more Pim kinases. Accordingly, these compounds are useful to treat conditions mediated by Pim kinase, such as the cancers and autoimmune disorders discussed herein.

Preferably, in the compounds of Formula I, Y represents a cyclic ether, e.g., a 5-6 membered ring containing one or two oxygen atoms as ring members, such as tetrahydropyran, tetrahydrofuran, dioxane, dioxolane, dihydropyran, dihyhydrofuran, and the like.

Another aspect of the present invention provides a method for treating a condition by modulation of Provirus Integration of Maloney Kinase (PIM Kinase), GSK3, KDR, PKC, KDR, PDGFRa, FGFR3, FLT3, or cABL activity comprising administering to a patient in need of such treatment an effective amount of a compound of Formula I or any of the various compounds of this type that are disclosed herein. A preferred embodiment of this aspect provides a method wherein the condition treated by modulation of PIM Kinase is a cancer selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

Yet another aspect of the present invention provides a pharmaceutical composition comprising a compound of Formula I, in its broadest and preferred embodiments including compounds of Formula IA, IB, IA′, IB′, II, and other variations thereof that are disclosed herein. The pharmaceutical composition comprises at least one pharmaceutically acceptable excipient, which is typically sterile. A preferred embodiment of this aspect provides a pharmaceutical composition comprising a compound of Formula I, in its broadest and preferred embodiments, wherein said pharmaceutical composition comprises an additional agent for the treatment of cancer. A further preferred embodiment of this aspect provides a pharmaceutical composition wherein the additional agent is selected from irinotecan, topotecan, gemcitabine, 5-fluorouracil, leucovorin carboplatin, cisplatin, taxanes, tezacitabine, cyclophosphamide, vinca alkaloids, imatinib (Gleevec), anthracyclines, rituximab, and trastuzumab.

A preferred aspect of the present invention provides a compound of Formula I having the following Formula II structure, or a stereoisomer, tautomer, or pharmaceutically acceptable salt thereof:

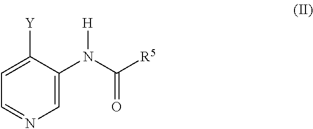

wherein,
Y is selected from tetrahydropyran, dioxane, dihydro-2H-pyran, dioxolane, dihydro-2H-pyran-4-(3H)-one, 5-methylenetetrahydro-2H-pyran-4-ol, 3,4-dihydro-2H-pyran-4-ol, 2H-pyran-4(3H)-one, and tetrahydrofuran, wherein each said Y group is independently substituted with at least one of R⁷, R⁸, R⁹, R¹⁰, R¹¹, R¹², R¹³, R¹⁴, and R¹⁵;
R⁵ is selected from a group consisting of thiazole, pyridine, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group, or C₃₋₈-heterocycloalkyl group;
R¹⁸, R¹⁹, and R²⁰ independently are selected from H, aryl, pyridine, thiazole, pyrimidine, pyrazine, pyridazine, amino, cyano, halogen, and C₁₋₄-alkyl, wherein said aryl, pyridine, thiazole, pyrimidine, pyridazine, and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, oxo, —SO₂—C₁₋₄ alkyl, CO—NH—C₃₋₆-branched alkyl, OC₁₋₄-alkyl, and OC₁₋₄-haloalkyl.

Another aspect of the present invention provides a method for treating a condition by modulation of Provirus Integration of Maloney Kinase (PIM Kinase), GSK3, PKC, KDR, PDGFRa, FGFR3, FLT3, or cABL activity comprising administering to a patient in need of such treatment an effective amount of a compound of Formula II. A preferred embodiment of this aspect provides a method wherein the condition treated by modulation of PIM Kinase is a cancer selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

Another aspect of the present invention provides a pharmaceutical composition comprising a compound of Formula II, with a preferred pharmaceutical composition comprising a compound of Formula II and an additional agent for the treatment of cancer. In a further preferred embodiment is provided a pharmaceutical composition wherein the additional agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib (Gleevec), anthracyclines, rituximab, and trastuzumab.

In other aspects, the present invention provides methods for treating Provirus Integration of Maloney Kinase (PIM Kinase) related disorders in a human or animal subject in need of such treatment comprising administering to said subject an amount of a compound of Formula I or II effective to inhibit PIM activity in the subject.

In yet other aspects, the present invention provides methods for treating PIM related disorders in a human or animal subject in need of such treatment comprising administering to said subject an amount of a compound of Formula I or II effective to reduce or prevent tumor growth in the subject in combination with at least one additional agent for the treatment of cancer.

Other aspects of the present invention provide therapeutic compositions comprising at least one compound of Formula I or II in combination with one or more additional agents for the treatment of cancer, as are commonly employed in cancer therapy.

The compounds of the invention are useful in the treatment of cancers, including hematopoietic malignancies, carcinomas (e.g., of the lungs, liver, pancreas, ovaries, thyroid, bladder or colon), melanoma, myeloid disorders (e.g., myeloid leukemia, multiple myeloma and erythroleukemia), adenomas (e.g., villous colon adenoma), sarcomas (e.g., osteosarcoma), autoimmune diseases, allergic reactions and in organ transplantation rejection syndromes.

The invention further provides compositions, methods of use, and methods of manufacture as described in the detailed description of the invention.

### DETAILED DESCRIPTION

One aspect of the present invention provides compounds of Formula I, and their stereoisomers, tautomers and pharmaceutically acceptable salts thereof:

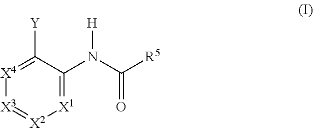

wherein,
X¹ represents CR¹ or N;
X² represents CR² or N;
X³ represents CR³ or N;
X⁴ represents CR⁴ or N; provided that not more than two of X¹, X², X³, and X⁴ can be N;
Y is selected from a group consisting of heterocyclo-alkyl, and partially unsaturated heterocyclo-alkyl, wherein each said Y group is independently substituted with at least one of R⁷, R⁸, R⁹, R¹⁰, R¹¹, R¹², R¹³, R¹⁴, and R¹⁵;
R¹, R², R³, and R⁴ independently are selected from the group consisting of hydrogen, halo, hydroxyl, nitro, cyano, SO₃H and substituted or unsubstituted alkyl, alkenyl, alkynyl, alkoxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, alkylthio, aryl, heteroaryl, cycloalkyl, hetero cycloalkyl, partially saturated cycloalkyl, aryloxy, heteroaryloxy, heterocyclyloxy, cycloalkyloxy, acyl, acylamino and acyloxy;
R⁵ is selected from a group consisting of thiazole, pyridine, pyrazole, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from hydroxy, hydroxy-C₁₋₄-alkyl, C₁₋₄-alkyl, H, D, C₁₋₄-halo-alkyl, C₁₋₄ alkoxy, —(CH₂)₁₋₄—X (where X is amino, C₁₋₄ alkoxy, hydroxy, F, Cl), amino, C₃₋₆-cycloalkyl, C₃₋₆ heterocyclo-alkyl, C₂₋₄ alkynyl, C₂₋₄ alkylene, (CH₂)₁₋₄—CN, (CH₂)₁₋₄—CONH₂, (CH₂)₁₋₄-CO₂H, carboxy, cyano, oxo, CONR₂ (where each R is independently H or C1-4 alkyl), and halogen; alternatively any two of R¹¹, R¹², R¹³, R¹⁴, and R¹⁵ along with the carbon atom or atoms that they are attached to can form a C₃₋₈-cycloalkyl or a C₃₋₈-heterocycloalkyl group that can be substituted with up to two groups selected from hydroxy, hydroxy-C₁₋₄-alkyl, C₁₋₄-alkyl, C₁₋₄-halo-alkyl, C₁₋₄ alkoxy, —(CH₂)₁₋₄—X (where X is amino, C₁₋₄ alkoxy, hydroxy, F, Cl), amino, C₂₋₄ alkynyl, C₂₋₄ alkylene, (CH₂)₁₋₄—CN, (CH₂)₁₋₄—CONH₂, (CH₂)₁₋₄—CO₂H, carboxy, cyano, oxo, CONR₂ (where each R is independently H or C₁₋₄ alkyl), and halogen; or two of R¹¹, R¹², R¹³, R¹⁴, and R¹⁵ when attached to the same carbon can form an exocyclic methylene (═CH₂);
R¹⁸, R¹⁹, and R²⁰ independently are selected from H, aryl, heteroaryl, hydroxy, amino, cyano, halogen, and C₁₋₆-alkyl, C₃₋₈-cycloalkyl, C₃₋₈-heterocycloalkyl, wherein said aryl, alkyl, heteroaryl, alkyl, cycloalkyl and heterocycloalkyl groups are further substituted with at least one of R²¹, R²², or R²³; and
R²¹, R²², and R²³ independently are selected from halogen, D, C₁₋₄-alkyl, amino, —NHC(O)—C₁₋₄ alkyl, COOH, hydroxy, oxo, CN, NO₂, H, CONH—C₁₋₄ alkyl, CO—NH—C₃₋₆-branched alkyl, —OC₁₋₄-alkyl, —SO₂—C₁₋₄ alkyl, —(CH₂)₁₋₄—X where X is OH, OMe, CN, or halo, and —OC₁₋₄-haloalkyl.

Typically, one of X¹, X², X³ and X⁴ is N; the remainder are optionally substituted carbon atoms as described above. Alternatively, two of these ring members may be N. Typically, two or all three of the others are CH.

Provided in one embodiment is a compound of Formula I wherein X₁ is N and X² is CR², X³ is CR³, and X⁴ is CR⁴. A preferred embodiment provides a compound of Formula I wherein X₂ is N and X¹ is CR¹, X³ is CR³, and X⁴ is CR⁴. Yet another preferred embodiment provides a compound of Formula I wherein X₃ is N and X¹ is CR¹, X² is CR², and X⁴ is CR⁴. Provided in another preferred embodiment is a compound of Formula I wherein X₄ is N and X¹ is CR¹, X² is N, and X³ is CR³. Yet another preferred embodiment provides a compound of Formula I, wherein X₁ is N and X² is CR², X³ is N, and X⁴ is CR⁴. Another embodiment provides a compound of Formula I, wherein X¹ represents CR¹; X² represents CR²; X³ represents CR³; and X⁴ represents CR⁴. Another embodiment provides a compound of Formula I, wherein X¹ represents CR¹; X² represents N; X³ represents CR³; and X⁴ represents N.

In the most preferred embodiments, X₂ is N and X¹ is CR¹, X³ is CR³, and X⁴ is CR⁴.

In some embodiments, each of R¹, R², R³ and R⁴ that is present represents H. In some embodiments, one of R¹, R², R³ and R⁴ that is present represents halo, Me, OMe, or OH, while the others each represent H.

In preferred embodiments, Y represents a cyclic ether such as a partially or fully saturated non-aromatic pyran or furan ring.

A further preferred embodiment provides a compound of Formula I, wherein Y is selected from a group consisting of tetrahydropyran, dioxane (particularly 1,3-dioxane), dioxolane, dihydro-2H-pyran, tetrahydrofuran, dihydro-2H-pyran-4(3H)-one, 5-methylenetetrahydro-2H-pyran-4-ol, 3,4-dihydro-2H-pyran-4-ol, and 2H-pyran-4(3H)-one wherein each said Y group is independently substituted with at least one of R⁷, R⁸, R⁹, R¹⁰, R¹¹, R¹², R¹³, R¹⁴, and R¹⁵. Compounds herein Y is tetrahydropyran, particularly 2-tetrahydropyranyl, are most preferred. Typically, Y is substituted with at least two and preferably three to five groups selected from OH, NH₂, and C₁₋₄ alkyl such as Me, Et or Propyl. It is typical that neither OH nor NH₂ is attached at the 2- or the 6-position of a tetrahydropyran or the 2- or 5-positions of a tetrahydrofuran, for example.

Another preferred embodiment provides a compound of Formula I, wherein R⁵ is selected from pyridine, pyrazine, pyrimidine, triazine, pyridone, pyridazinone, and thiazole, wherein each said R⁵ group is substituted with one to three substituents selected from R¹⁸, R¹⁹, and R²⁰ as described herein. Typically, R⁵ is substituted with at least one group selected from aryl, heteroaryl, amino, cyano, halogen, and C₁₋₆-alkyl, C₃₋₈-cycloalkyl, C₃₋₈-heterocycloalkyl, wherein said aryl, alkyl, heteroaryl, alkyl, cycloalkyl and heterocycloalkyl groups are further substituted with at least one of R²¹, R²², or R²³; suitable heteroaryl groups that can be present as R¹⁸, R¹⁹, or R²⁰ include thiazole, pyrazole, pyridine, and pyrimidine and bicyclic groups such as azaindole, benzopyrazole, benzothiazole, and the like. Suitable aryl groups for R⁵ include phenyl, or fused ring systems such as indole, benzothiazole, benzopyrazole or benzimidazole when attached to R⁵ through the phenyl ring. These heteroaryl and aryl groups are optionally substituted with one or more, typically one to three, R²¹, R²², or R²³.

In some embodiments, R⁵ is selected from 2-pyridyl, 4-pyrimidinyl, 2-pyrazinyl, and 4-thiazolyl; ring numbering here reflects the point of attachment of R⁵ to the carbonyl shown in Formula I and does not take into account other substituents (e.g., R¹⁹, and R²⁰) that may be present on R⁵.

Particularly preferred are compounds wherein R⁵ is substituted with a phenyl group, and the phenyl group is substituted by up to three groups as described herein; and R⁵ may be further substituted with halo, cyano, and/or amino. Preferred groups selected for substituents on a phenyl ring attached to R⁵ include halo (e.g., F or Cl), C₁₋₄ alkyl or alkoxy, C₁₋₄ alkylsulfonyl, and the like.

Yet another preferred aspect provides a compound of Formula I wherein R⁷ represents H, trifluoromethyl, trifluoro-ethyl, D, fluoro, methyl, or ethyl. Typically in these embodiments, R⁷ is attached to the ring carbon of group Y that is attached to the ring in Formula I containing X¹ to X⁴ as ring atoms. In some embodiments of these compounds, the ring carbon of group Y that is attached to the ring in Formula I containing X¹ to X⁴ as ring atoms is position 2 of a tetrahydropyran ring.

Yet another preferred aspect of the present invention provides a compound of Formula I wherein R⁸, R⁹, R¹⁰, R¹¹, R¹², R¹³, R¹⁴, and R¹⁵ independently are selected from H, hydroxy, D, hydroxy-methyl, Cl, chloro-methyl, F, methyl, ethyl, amino, ethylene, oxo, fluoromethyl, difluoromethyl, trifluoromethyl, vinyl, acetylene, cyano and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl or a C₃₋₈-heterocycloalkyl group. In some embodiments, at least two and preferably three of R⁸, R⁹, R¹⁰, R¹¹, R¹², R¹³, R¹⁴, and R¹⁵ are selected from hydroxy, amino, methyl, ethyl, propyl, halo (F, Cl) and C₁₋₄ haloalkyl.

A further preferred aspect of the present invention provides a compound of Formula I wherein R¹⁸, R¹⁹, and R²⁰ independently are selected from H, hydroxy, phenyl, pyridine, thiazole, pyrimidine, pyrazine, pyridazine, amino, cyano, halogen, C₃₋₄-cycloalkyl or a C₃₋₄-heterocycloalkyl, and C₁₋₄-alkyl, wherein said phenyl, pyridine, thiazole, pyrimidine, pyrazine, pyridazine, amino, C₃₋₆-cycloalkyl or a C₃₋₆-heterocycloalkyl, and C₁₋₄-alkyl groups are further substituted with at least one of R²¹, R²², and R²³; and R²¹, R²², and R²³ independently are selected from halogen, C₁₋₄-alkyl, hydroxy, amino, CN, NO₂, H, COOH, CONH—C₁₋₄ alkyl, CO—NH—C₃₋₄-branched alkyl, OC₁₋₂-alkyl, and OC₁₋₂-haloalkyl; or optionally, two of R²¹, R²² and R²³ can be taken together to form a 5-6 membered ring that may contain one or two O, N or S as ring members and can be substituted with 1-2 groups selected from oxo, halo, Me, Et, cyclopropyl, OMe, OH, NH₂, and CN.

In another aspect, the invention provides a compound of Formula IA or IB:

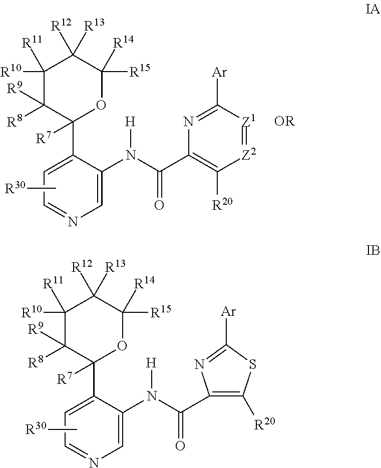

wherein:
Z¹ is N or C—Y, where Y is H, NH₂, F, Cl, or CN;
Z² is CH or N;
R²⁰ is H, H halo, OH, or NH₂;
R³⁰ is H, Me, OMe, CN, or halo;
R⁷ is H, Me or CF₃;
R⁸ and R⁹ are independently H, Me, OH, NH₂, OMe, or F; or R⁸ and R⁹ taken together represent ═O (oxo):
or R⁷ and R⁸ taken together form a double bond between the carbon atoms to which they are attached;
R¹⁰ and R¹¹ are independently H, C₁₋₄ alkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹⁰ and R¹¹ are linked together to form a 3-6 membered cycloalkyl or heterocycloalkyl ring; or R¹⁰ and R¹¹ taken together represent ═O (oxo) or ═CH₂:
R¹² and R¹³ are independently H, C₁₋₄ alkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹² and R¹³ are linked together to form a 3-6 membered cycloalkyl or heterocycloalkyl ring; or R¹² and R¹³ taken together represent ═O (oxo) or ═CH₂:
R¹⁴ and R¹⁵ are independently H, C₁₋₄ alkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹⁴ and R¹⁵ are linked together to form a 3-6 membered cycloalkyl or heterocycloalkyl ring;
   where each X is independently F, Cl, CN, OH, OMe, or NH₂;
   and optionally R¹² can be taken together with either R¹¹ or R¹⁴ to form a 5-6 membered ring containing up to 2 heteroatoms selected from N, O and S as ring members, and optionally substituted with ═O, CN, halo, Me, OMe, OH, or NH₂;
Ar is selected from phenyl, pyridyl, pyrazinyl, pyridazinyl, thiazolyl, and pyrazolyl, where Ar is optionally substituted with up to four groups selected from halo, C₁₋₄ alkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, —(CH₂)₁₋₃—OR,
   wherein each R is H or C₁₋₄ alkyl;
including the tautomers, stereoisomers, and pharmaceutically acceptable salts of these compounds.

In some embodiments of these compounds of Formula IA or IB, Z¹ is N; in alternative embodiments, Z¹ is C—Y, where Y is typically H, F or CN. When Z¹ is C—Y, Z² is sometimes N. When Z¹ is N, Z² is typically CH.

In the compounds of Formula IA or IB, R²⁰ is preferably H or NH₂.

In embodiments of compounds of Formula IA or IB, R³⁰ is preferably H.

In the compounds of Formula IA and IB, Ar is preferably phenyl. In some such embodiments, Ar is unsubstituted. In other such embodiments, Ar is substituted with one or two F (fluorine) groups, and preferred embodiments of Ar include unsubstituted phenyl, 2-fluorophenyl, and 2,6-difluorophenyl. In some embodiments, Ar is 2-fluorophenyl or 2,6-difluorophenyl that is substituted with at least one and optionally two additional group selected from C₁₋₄ alkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, or a group of the formula —(CH₂)₁₋₃—OR, or where two such groups joined together form a 5-6 membered ring fused to Ar, optionally containing one or two N, O or S as ring members and optionally substituted as described herein;
wherein each R is H or C₁₋₄ alkyl, and where two R on the same or adjacent connected atoms can be joined together to form a 5-6 membered ring containing up to two heteroatoms selected from N, O and S as ring members.

In many embodiments of the foregoing compounds of Formula IA or IB, R⁷ is H. In alternative embodiments, R⁷ is CF₃.

In some embodiments of the foregoing compounds of Formula IA or IB, R⁸ is H, and R⁹ is selected from H, OH, F, and Me. In many embodiments, R⁸ and R⁹ are both H.

In some embodiments of the compounds of Formula IA and IB, at least one of R¹⁰, R¹¹, R¹², R¹³, R¹⁴ and R¹⁵ is selected from —OH, NH₂, and C₁₋₄ alkyl. In preferred embodiments, at least two of R¹⁰, R¹¹, R¹², R¹³, R¹⁴ and R¹⁵ are selected from —OH, NH₂, Me, and Et. In many such embodiments, at least three of R¹⁰, R¹¹, R¹², R¹³, R¹⁴ and R¹⁵ are selected from —OH, NH₂, Me, and Et. Preferably, at least two of R¹⁰, R¹¹, R¹², R¹³, R¹⁴ and R¹⁵ represent H. In some preferred embodiments, the compound is of one of these formulas:

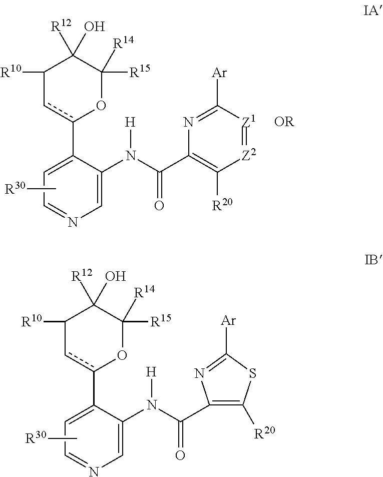

where R¹⁰ is OH or NH₂; R²⁰ is H or NH₂; R³⁰ is H; R¹² is H, Me, Et, or Propyl; and R¹⁴ is selected from H, Me, Et, vinyl, propyl, and —(CH₂)₁₋₃—X, where X is OH, CN, OMe, or halo (particularly F or Cl) while R¹⁵ is H or Me; or R¹⁴ and R¹⁵ taken together form a spirocyclopropane ring; and the other variable groups (Ar, Z¹, Z², etc.) are as defined above for Formulas IA and IB. The dashed lines in Formulas IA′ and IB′ represent an optional carbon-carbon double bond, i.e., the bond represented by the linkage including the dashed line can be either a single bond or a double bond.

In a preferred embodiment, the compounds of Formula IA′ and IB′ are enriched in one stereoisomer, diastereomer or optical isomer of the tetrahydropyran ring, with the major isomer having this stereochemistry:

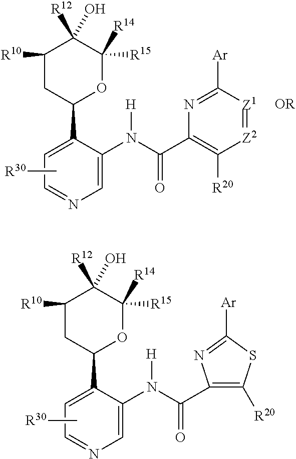

where R¹⁰, R¹², R¹⁴, R¹⁵, R²⁰, R³⁰, Z¹ and Z² and Ar are as defined for Formula IA′ and IB′ above.

Preferably, these compounds are used as a single diastereomer with regard to substitution on the tetrahydropyran ring; optionally, they are used as a single optical isomer (enantiomer). It is understood that ‘single diastereomer’ or ‘single optical isomer’ means that other isomers have been substantially removed, thought they may still be present in small amounts. Typically, the compound will be at least 90% one isomer, preferably at least 95% one isomer.

Another aspect of the present invention provides a method for treating a condition by modulation of Provirus Integration of Maloney Kinase (PIM Kinase), GSK3, KDR, PKC, PDGFRa, FGFR3, FLT3, or cABL activity comprising administering to a patient in need of such treatment an effective amount of a compound of Formula I (including IA, IB, IA′, and IB′ and the disclosed variations thereof). A preferred embodiment of this aspect provides a method wherein the condition treated by modulation of PIM Kinase is a cancer selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

Yet another aspect of the present invention provides a pharmaceutical composition comprising a compound of Formula I, in its broadest and preferred embodiments. A preferred embodiment of this aspect provides a pharmaceutical composition comprising a compound of Formula I, in its broadest and preferred embodiments, wherein said pharmaceutical composition comprises an additional agent for the treatment of cancer. A further preferred embodiment of this aspect provides a pharmaceutical composition wherein the additional agent is selected from irinotecan, topotecan, gemcitabine, 5-fluorouracil, cytarabine, daunorubicin, PI3 Kinase inhibitors, mTOR inhibitors, DNA synthesis inhibitors, leucovorin carboplatin, cisplatin, taxanes, tezacitabine, cyclophosphamide, vinca alkaloids, imatinib (Gleevec), anthracyclines, rituximab, and trastuzumab.

A preferred aspect of the present invention provides a compound of Formula I having the following Formula II structure, or a stereoisomer, tautomer, or pharmaceutically acceptable salt thereof:

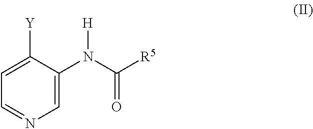

wherein,
Y is selected from tetrahydropyran, dioxane, dihydro-2H-pyran, dioxolane, dihydro-2H-pyran-4-(3H)-one, 5-methylenetetrahydro-2H-pyran-4-ol, 3,4-dihydro-2H-pyran-4-ol, 2H-pyran-4(3H)-one, and tetrahydrofuran, wherein each said Y group is independently substituted with at least one of R⁷, R⁸, R⁹, R¹⁰, R¹¹, R¹², R¹³, R¹⁴, and R¹⁵;
R⁵ is selected from a group consisting of thiazole, pyridine, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group, or C₃₋₈-heterocycloalkyl group;
R¹⁸, R¹⁹, and R²⁰ independently are selected from H, aryl, pyridine, thiazole, pyrimidine, pyrazine, pyridazine, amino, cyano, halogen, and C₁₋₄-alkyl, wherein said aryl, pyridine, thiazole, pyrimidine, pyridazine, and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

A preferred aspect of this embodiment provides a compound of Formula II wherein:
Y represents tetrahydropyran, or dihydro-pyran, wherein each said Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

Yet another preferred aspect of this invention provides a compound of Formula II wherein:
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl₁ group;
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₆ cycloalkyl, C₃₋₆ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, oxo, —SO₂—C₁₋₄ alkyl, CO—NH—C₃₋₆-branched alkyl, OC₁₋₄-alkyl, and OC₁₋₄-haloalkyl.

Yet another preferred embodiment of the present invention provides a compound of Formula II, wherein:
Y represents dioxane or dioxolane, wherein each y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

A preferred aspect of this embodiment provides a compound of Formula II wherein:
R⁵ is selected from a group consisting of thiazole, pyridine, pyrimidine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, triazine, amino, cyano, halogen, C₃₋₆ cycloalkyl, C₃₋₆ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

A further preferred aspect provides a compound of Formula II, wherein:
Y represents tetrahydrofuran, or dihydro-2H-pyran-4(3H)-one, wherein each Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

A further preferred embodiment of this aspect provides a compound of Formula II, wherein:
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₆ cycloalkyl, C₃₋₆ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

Another aspect of the present invention provides a method for treating a condition by modulation of Provirus Integration of Maloney Kinase (PIM Kinase), GSK3, PKC, KDR, PDGFRa, FGFR3, FLT3, or cABL activity comprising administering to a patient in need of such treatment an effective amount of a compound of Formula II. A preferred embodiment of this aspect provides a method wherein the condition treated by modulation of PIM Kinase is a cancer selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

Another aspect of the present invention provides a pharmaceutical composition comprising a compound of Formula II, with a preferred pharmaceutical composition comprising a compound of Formula II and an additional agent for the treatment of cancer. In a further preferred embodiment is provided a pharmaceutical composition wherein the additional agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib (Gleevec), anthracyclines, rituximab, and trastuzumab.

The compounds of the invention are useful in the treatment of cancers, including hematopoietic malignancies, carcinomas (e.g., of the lungs, liver, pancreas, ovaries, thyroid, bladder or colon), melanoma, myeloid disorders (e.g., myeloid leukemia, multiple myeloma and erythroleukemia), adenomas (e.g., villous colon adenoma), sarcomas (e.g., osteosarcoma), autoimmune diseases, allergic reactions and in organ transplantation rejection syndromes.

In yet another aspect of the present invention is provided a use of a compound of Formula I or II for preparing a medicament for treating a condition by modulation of Provirus Integration of Maloney Kinase (PIM Kinase) activity. In a preferred embodiment of this aspect of the invention the condition is a cancer selected from carcinoma of the lungs, pancreas, thyroid, ovarian, bladder, breast, prostate, or colon, melanoma, lymphoma, myeloid leukemia, multiple myeloma and erythro leukemia, villous colon adenoma, and osteosarcoma.

In another aspect, the present invention relates to methods of inhibiting the activity of at least one kinase selected from the group consisting of Pim1, Pim2, Pim3, GSK3, KDR, PKC, PDGFRa, FGFR3, FLT3, and cABL315T in a subject, or treating a biological condition mediated by at least one of Pim1, Pim2, Pim3, GSK3, KDR, PDGFRa, FGFR3, FLT3, PKC and cABL315T, in a human or animal subject in need of such treatment, comprising administering to the subject at least one compound of Formula I or II in an amount effective to inhibit the kinase in the subject. The therapeutic compounds are useful for treating patients with a need for such inhibitors (e.g., those suffering from diseases mediated by abnormal serine/threonine kinase receptor signaling).

The following enumerated embodiments disclose specific realizations of the invention:

1. A compound of Formula I, or a pharmaceutically acceptable salt thereof,

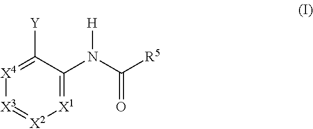

wherein,
X¹ represents CR¹ or N;
X² represents CR² or N;
X³ represents CR³ or N;
X⁴ represents CR⁴ or N; provided that not more than two of X¹, X², X³, and X⁴ can be N;
Y is selected from a group consisting of heterocyclo-alkyl, and partially unsaturated heterocyclo-alkyl, wherein each said Y group is independently substituted with at least one of R⁷, R⁸, R⁹, R¹⁰, R¹¹, R¹², R¹³, R¹⁴, and R¹⁵;
R¹, R², R³, and R⁴ independently are selected from the group consisting of hydrogen, deuterium, halo, hydroxyl, nitro, cyano, SO₃H and substituted or unsubstituted alkyl, alkenyl, alkynyl, alkoxy, amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, sulfonyl, sulfonyloxy, thioacyl, thiol, alkylthio, aryl, heteroaryl, cycloalkyl, hetero cycloalkyl, partially saturated cycloalkyl, aryloxy, heteroaryloxy, heterocyclyloxy, cycloalkyloxy, acyl, acylamino and acyloxy;
R⁵ is selected from a group consisting of thiazole, pyridine, pyrimidine, triazine, pyrazole, pyridazinone, pyridone, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from hydroxy, hydroxy-C₁₋₄-alkyl, C₁₋₄-alkyl, H, D, C₁₋₄-halo-alkyl, C₁₋₄ alkoxy, amino, C₃₋₆-cycloalkyl, C₃₋₆ heterocyclo-alkyl, C₂₋₄ alkynyl, C₂₋₄ alkylene, (CH₂)₁₋₄—CN, (CH₂)₁₋₄—CONH₂, (CH₂)₁₋₄—CO₂H, carboxy, cyano, oxo, CONR₂ and halogen; alternatively any two of R¹¹, R¹², R¹³, R¹⁴, and R¹⁵ along with the carbon atom or atoms that they are attached to can form a C₃₋₈-cycloalkyl or a C₃₋₈-heterocycloalkyl group;
R¹⁸, R¹⁹, and R²⁰ independently are selected from H, D, aryl, amino, cyano, halogen, and C₁₋₆-alkyl, C₃₋₈-cycloalkyl, C₃₋₈-heterocycloalkyl, wherein said aryl, alkyl, heteroaryl, alkyl, cycloalkyl and heterocycloalkyl groups are further substituted with at least one of R²¹, R²², or R²³; and
R²¹, R²², and R²³ independently are selected from halogen, C₁₋₄-alkyl, amino, COOH, hydroxy, CN, NO₂, H, D, CONH—C₁₋₄ alkyl, CO—NH—C₃₋₆-branched alkyl, OC₁₋₄-alkyl, and OC₁₋₄-haloalkyl.

Specific embodiments of special interest include each of the particular compounds depicted in Table 1.

2. A compound of Embodiment 1 wherein X¹ is N and X² is CR², X³ is CR³, and X⁴ is CR⁴.

3. A compound of Embodiment 1 wherein X² is N and X¹ is CR¹, X³ is CR³, and X⁴ is CR⁴. This is a preferred embodiment, particularly when R¹, R³ and R⁴ each represent H.

4. A compound of Embodiment 1 wherein X³ is N and X¹ is CR¹, X² is CR², and X⁴ is CR⁴.

5. A compound of Embodiment 1 wherein X⁴ is N and X¹ is CR¹, X² is N, and X³ is CR³.

6. A compound of Embodiment 1 wherein X¹ is N and X² is CR², X³ is N, and X⁴ is CR⁴.

7. A compound of Embodiment 1, wherein:
X¹ represents CR¹;
X² represents CR²;
X³ represents CR³; and
X⁴ represents CR⁴.

8. A compound of any of embodiments 1-7, wherein Y is selected from a group consisting of tetrahydropyran, dioxane, dioxolane, dihydro-2H-pyran, tetrahydrofuran, dihydro-2H-pyran-4(3H)-one, 5-methylenetetrahydro-2H-pyran-4-ol, 3,4-dihydro-2H-pyran-4-ol, and 2H-pyran-4(3H)-one wherein each said Y group is independently substituted with at least one of R⁷, R⁸, R⁹, R¹⁰, R¹¹, R¹², R¹³, R¹⁴, and R¹⁵. Frequently, Y is a tetrahydropyran ring. In preferred compounds of this embodiment, Y is tetrandyropyran or dihydro-2H-pyran, such as 2-tetrahydropyran or dihydro-2H-pyran-6-yl, and is substituted by at least two groups selected from OH, NH₂, C₁₋₄ alkyl, halo, C₁₋₄ haloalkyl, and —(CH₂)₁₋₃X, where X is halo, amino, CN, cyclopropyl, hydroxy, or methoxy.

9. A compound of Embodiment 1, 2, 3, 4, 5, 6, 7 or 8 wherein R⁵ is selected from pyridine, pyrazine, pyrimidine, triazine, and thiazole, particularly 2-pyridinyl, or 4-pyrimidinyl, or 2-thiazolyl (where the carbonyl shown in Formula I is attached to the named ring at the 2-position, 4-position, or 2-position, respectively), wherein each said R⁵ group is substituted with one to three substituents selected from R¹⁸, R¹⁹, and R²⁰. In particularly preferred compounds of this embodiment, R⁵ is pyridine, pyrimidine, or thiazole and is optionally substituted with NH₂ or halo or both.

10. A compound of Embodiment 1, 2, 3, 4, 5, 6, 7 or 8 or 9, wherein R⁷ represents H, trifluoromethyl, trifluoro-ethyl, D, fluoro, methyl, or ethyl. R⁷ in these embodiments is preferably located on the carbon atom of ring Y that is attached to the ring in Formula I that contains X¹-X⁴. Exemplary compounds have this substructure:

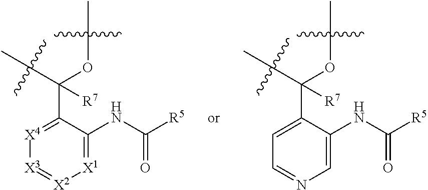

and can be further substituted as described for Formula I.

11. A compound of Embodiment 1, 2, 3, 4, 5, 6, 7, 8, or 9 or 10, wherein R⁸, R⁹, R¹⁰, R¹¹, R¹², R¹³, R¹⁴, and R¹⁵ independently are selected from H, hydroxy, D, hydroxy-methyl, Cl, chloro-methyl, F, methyl, ethyl, amino, ethylene, oxo, fluoromethyl, difluoromethyl, trifluoromethyl, vinyl, acetylene, cyano and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl or a C₃₋₈-heterocycloalkyl group. Preferably, 2, 3 or 4 of the groupr represented by R⁸, R⁹, R¹⁰, R¹¹, R¹², R¹³, R¹⁴, and R¹⁵ are other than H, and the others all represent H. Commonly R⁷ is H. Frequently, 2, 3 or 4 of R⁸, R⁹, R¹⁰, R¹¹, R¹², R¹³, R¹⁴, and R¹⁵ are selected from amino, hydroxy, methyl, and ethyl, and at least one of these represents either hydroxy or amino.

12. A compound of Embodiment 1, 2, 3, 4, 5, 6, 7, 8, 9, or 10 or 11, wherein R¹⁸, R¹⁹, and R²⁰ independently are selected from H, phenyl, pyridine, thiazole, pyrimidine, pyrazine, pyridazine, amino, cyano, halogen, C₃₋₆-cycloalkyl or a C₃₋₆-heterocycloalkyl, and C₁₋₄-alkyl, wherein said phenyl, pyridine, thiazole, pyrimidine, pyrazine, pyridazine, amino, C₃₋₈-cycloalkyl or a C₃₋₆-heterocycloalkyl, and C₁₋₄-alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₄-branched alkyl, OC₁₋₂-alkyl, and OC₁₋₂-haloalkyl. In preferred compounds of this embodiment, R¹⁸ and R¹⁹ are selected from H, halo and amino; and R²⁰ is optionally substituted phenyl. Preferably, the phenyl group is substituted with one or two fluoro substituents, and optionally an additional group selected from C₁₋₄-alkyl, hydroxy, amino, CN, NO₂, COOH, CONH—C₁₋₄ alkyl, CO—NH—C₃₋₄-branched alkyl, OC₁₋₂-alkyl, and OC₁₋₂-haloalkyl.
R¹⁸, R¹⁹, and R²⁰ are substituent groups on R⁵; typically one of these is an aryl or heteroaryl ring selected from the ones named above, and preferably one of them is phenyl that is itself further substituted with at least one of R²¹, R²², and R²³. The other two of R¹⁸, R¹⁹, and R²⁰ typically represent H, amino or F, and preferably they are different from each other unless both represent H. In some preferred embodiments, one is H and the other is F; in other preferred embodiments, one of them is H and the other is NH₂.

13. A compound of Embodiment 1, which is of Formula IA or IB:

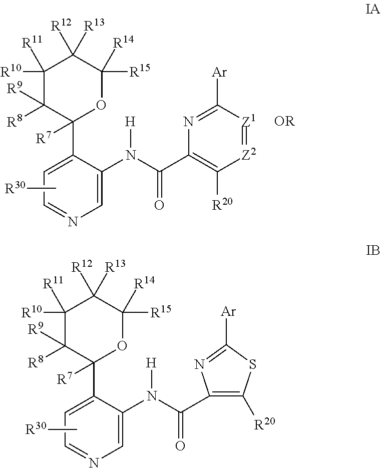

wherein:
Ar is selected from phenyl, pyridyl, pyrazinyl, pyridazinyl, thiazolyl, and pyrazolyl, where Ar is optionally substituted with up to four groups selected from halo, C₁₋₄ alkyl, C₃₋₅ cycloalkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, —(CH₂)₁₋₃—OR, wherein each R is H or C₁₋₄ alkyl or C₃₋₅ cycloalkyl;
Z¹ is N or C—Y, where Y is H, NH₂, F, Cl, or CN;
Z² is CH or N;
R²⁰ is H, D, halo, OH, or NH₂;
R³⁰ is H, D, Me, OMe, CN, or halo;
R⁷ is H, D, Me or CF₃;
R⁸ and R⁹ are independently H, D, Me, OH, NH₂, OMe, or F; or R⁸ and R⁹ taken together represent ═O (oxo):
   or R⁷ and R⁸ taken together form a double bond between the carbon atoms to which they are attached;
R¹⁰ and R¹¹ are independently H, D, C₁₋₄ alkyl, C₃₋₅ cycloalkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹⁰ and R¹¹ are linked together to form a 3-6 membered cycloalkyl or heterocycloalkyl ring; or R¹⁰ and R¹¹ taken together represent ═O (oxo) or ═CH₂:
R¹² and R¹³ are independently H, D, C₁₋₄ alkyl, C₃₋₅ cycloalkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹² and R¹³ are linked together to form a 3-6 membered cycloalkyl or heterocycloalkyl ring; or R¹² and R¹³ taken together represent ═O (oxo) or ═CH₂:
R¹⁴ and R¹⁵ are independently H, D, C₁₋₄ alkyl, C₃₋₅ cycloalkyl, C₁₋₄alkoxy, C₁₋₄ haloalkyl, C₂₋₄ alkenyl, C₂₋₄ alkynyl, —(CH₂)₁₋₃X, OH, NH₂, or F; or R¹⁴ and R¹⁵ are linked together to form a 3-6 membered cycloalkyl or heterocycloalkyl ring;
where each X is independently F, Cl, CN, OH, OMe, or NH₂;
and optionally R¹² can be taken together with either R¹¹ or R¹⁴ to form a 5-6 membered ring containing up to 2 heteroatoms selected from N, O and S as ring members, and optionally substituted with one or two groups selected from ═O (oxo), CN, halo, Me, OMe, OH, and NH₂;
including the tautomers, stereoisomers, and pharmaceutically acceptable salts of these compounds.
Typically in these compounds, R⁷ is H. In some embodiments, R⁸ and R⁹ each represent H, also, in many embodiments. Alternatively, R⁷ and R⁸ together represent a carbon-carbon double bond between the carbon atoms to which they are attached. In such compounds, R⁹ is typically H or Me.
Typically, at least two and preferably three or four of the groups R¹⁰, R¹¹, R¹², R¹³, R¹⁴ and R¹⁵ are selected from amino, hydroxy, methyl, ethyl, propyl, CN, halomethyl, and hydroxymethyl; frequently, the remainder of these groups represent H.
In preferred compounds of this embodiment, Ar is optionally substituted phenyl. In some such embodiments, the phenyl group is substituted with one or two fluoro substituents, and optionally an additional group selected from C₁₋₄-alkyl, hydroxy, amino, C₁₋₄ alkyl sulfonyl, CN, NO₂, COOH, CONH—C₁₋₄ alkyl, CO—NH—C₃₋₄-branched alkyl, OC₁₋₂-alkyl, and OC₁₋₂-haloalkyl.

14. The compound of Formula IA in embodiment 13, wherein Z¹ is N; or Z¹ is C—Y, where Y is H, F or CN. Typically, Z² is CH or N, preferably CH.

15. The compound of Embodiment 13 or 14, wherein R²⁰ is H or NH₂.

16. The compound of Embodiment 13 or 14 or 15, wherein R³⁰ is H.

17. The compound of any of Embodiments 13-16, wherein Ar is unsubstituted phenyl, or Ar is either 2-fluorophenyl or 2,6-difluorophenyl that is optionally substituted with one or two additional groups selected from halo, C₁₋₄ alkyl, C₁₋₄alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, and a group of the formula —(CH₂)₁₋₃—OR, or two such groups can be joined together to form a 5-6 membered optionally substituted ring fused to Ar and containing up to two heteroatoms selected from N, O and S as ring members;
wherein each R is independently H or C₁₋₄ alkyl, and where two R on the same or adjacent connected atoms can be joined together to form a 5-6 membered ring containing up to two heteroatoms selected from N, O and S as ring members.
In preferred embodiments, R is Me in the group —SO₂R.

18. The compound of Embodiment 17, wherein at least two of R¹⁰, R¹¹, R¹², R¹³, R¹⁴ and R¹⁵ are selected from —OH, NH₂, Me, and Et; typically, 0 or 1 one of them represents NH₂, and no two of R¹⁰, R¹¹, R¹², R¹³, R¹⁴ and R¹⁵ that are on the same carbon atom represent either OH or NH₂.

19. The compound of Embodiment 13, which is a compound of Formula IA′ or IB′:

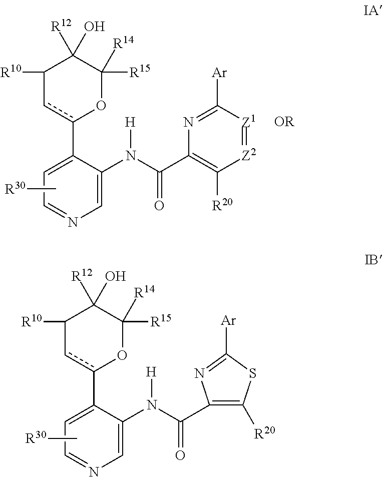

wherein the dashed line represents an optional carbon-carbon double bond;
R²⁰ is H or NH₂;
R³⁰ is H;
R¹⁰ is OH or NH₂;
R¹² is H, Me, Et, or Propyl;
R¹⁴ is selected from H, Me, Et, vinyl, propyl, isopropyl, t-butyl, cyclopropyl and —(CH₂)₁₋₃—X, where X is OH, CN, OMe, or halo, and R¹⁵ is H or Me;
or R¹⁴ and R¹⁵ taken together form a spirocyclopropane ring.

20. The compound of Embodiment 19, which is of the formula:

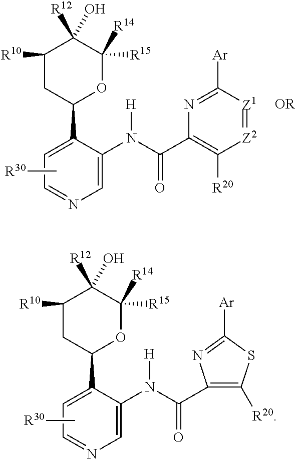

In these compounds, R¹⁰ is preferably OH or NH₂; R¹² is preferably H or Me; R¹⁴ is preferably Me or Et; R¹⁵ is preferably H; and R³⁰ is preferably H. Typically, Ar is unsubstituted phenyl, or Ar is 2-fluorophenyl or 2,6-difluorophenyl and is optionally substituted with one or two additional groups selected from halo, C₁₋₄ alkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, and a group of the formula —(CH₂)₁₋₃—OR, or two such groups can be joined together to form a 5-6 membered optionally substituted ring fused to Ar and containing up to two heteroatoms selected from N, O and S as ring members;
wherein each R is independently H or C₁₋₄ alkyl, and where two R on the same or adjacent connected atoms can be joined together to form a 5-6 membered ring containing up to two heteroatoms selected from N, O and S as ring members.

21. A compound of Formula II, or a pharmaceutically acceptable salt thereof,

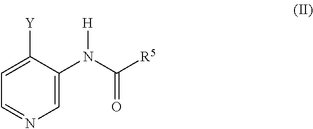

wherein,
Y is selected from tetrahydropyran, dioxane, dihydro-2H-pyran, dioxolane, dihydro-2H-pyran-4-(3H)-one, 5-methylenetetrahydro-2H-pyran-4-ol, 3,4-dihydro-2H-pyran-4-ol, 2H-pyran-4(3H)-one, and tetrahydrofuran, wherein each said Y group is independently substituted with at least one of R⁷, R⁸, R⁹, R¹⁰, R¹¹, R¹², R¹³, R¹⁴, and R¹⁵;
R⁵ is selected from a group consisting of thiazole, pyridine, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group, or C₃₋₈-heterocycloalkyl group;
R¹⁸, R¹⁹, and R²⁰ independently are selected from H, aryl, pyridine, thiazole, pyrimidine, pyrazine, pyridazine, amino, C₃₋₈-cycloalkyl or a C₃₋₈-heterocycloalkyl, cyano, halogen, and C₁₋₄-alkyl, wherein said aryl, pyridine, thiazole, pyrimidine, pyrazine, pyridazine, amino and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

22. The compound of Embodiment 21, wherein:
Y represents tetrahydropyran, or dihydro-pyran, wherein each said Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

23. The compound of Embodiment 21 or 22, wherein Y represents tetrahydropyran. Preferably, this tetrahydropyran is attached via its position 2 to the aromatic ring shown in Formula I.

24. The compound of Embodiment 21 or 22, wherein Y represents dihydro-pyran. Preferably, this dihydropyran is attached via its position 2 to the aromatic ring shown in Formula I.

25. The compound of any one of Embodiments 21-24, wherein:
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, R⁹, R¹⁰, trifluoromethyl, vinyl, acetylene, and cyano-methyl; alternatively any two of R⁸, R¹¹, R¹², R¹³, R¹⁴, and R¹⁵ along with the carbon atom to which they are attached can be taken together to form a C₃₋₈-cycloalkyl group or C₃₋₈-heterocycloalkyl₁ group. Typically, 2-5 of these represent a group selected from Me, Et, OH, and NH₂, while the remaining ones each represent H.

26. The compound of any one of Embodiments 21-25, wherein:
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₆ cycloalkyl, C₃₋₆ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

In preferred compounds of this type, R⁵ is selected from thiazole, pyridine and pyrimidine, and is attached to the carbonyl shown in Formula II at position 2 of the thiazole or pyridine, or at position 4 of the pyrimidine.

27. The compound of Embodiment 21, wherein:
Y represents tetrahydrofuran, or dihydro-2H-pyran-4(3H)-one, wherein each Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group. Typically, 2-5 of these groups represent a substituent selected from Me, Et, OH, and NH₂, while the remaining ones each represent H.

28. The compound of Embodiment 21 or 27, wherein:
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₈ cycloalkyl, C₃₋₈ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

29. A pharmaceutical composition comprising a compound of any of Embodiments 1-28 admixed with at least one pharmaceutically acceptable excipient.

30. The pharmaceutical composition of Embodiment 29, wherein said pharmaceutical composition comprises an additional agent for the treatment of cancer.

31. The pharmaceutical composition of Embodiment 30 wherein the additional agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib (Gleevec), anthracyclines, rituximab, and trastuzumab.

32. A method for treating a condition by modulation of Provirus Integration of Maloney Kinase (PIM Kinase), GSK3, PKC, KDR, PDGFRa, FGFR3, FLT3, or cABL activity comprising administering to a patient in need of such treatment an effective amount of a compound of any of Embodiments 1-28, or a pharmaceutical composition of Embodiment 29.

33. The method of Embodiment 32 wherein the condition is selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

34. The method of Embodiment 32, wherein the condition is an autoimmune disorder selected from Crohn's disease, inflammatory bowel disease, rheumatoid arthritis, and chronic inflammatory diseases.

35. A compound of any of Embodiments 1-28, for use in the treatment of cancer or an autoimmune disorder, or for use as a medicament. Similarly, this embodiment includes use of a compound of any of Embodiments 1-28 for manufacture of a medicament.

36. The compound of Embodiment 35, wherein the cancer is selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

37. The compound of Embodiment 35, wherein the autoimmune disorder is selected from Crohn's disease, inflammatory bowel disease, rheumatoid arthritis, and chronic inflammatory diseases.

### Definitions

“PIM inhibitor” is used herein to refer to a compound that exhibits an IC₅₀ with respect to PIM Kinase activity of no more than about 100 μM and more typically not more than about 50 μM, as measured in the PIM depletion assays described hereinbelow. Preferably for use in the methods described herein or for use as a medicament, the compound exhibits an IC₅₀ with respect to PIM Kinase less than 1 μM when measured by the methods described herein.

The phrase “alkyl”, as used here in, refers to an alkyl group containing 1 to 12 carbon atoms. Illustrative examples are straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups. Illustrative examples are CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and —CH(C₂H₅)CH(CH₃)CH(CH₃)(CH₂CH₃). Thus the phrase ‘alkyl group’ includes primary alkyl groups, secondary alkyl groups, and tertiary alkyl groups. Preferred alkyl groups include C₁₋₄ straight chain alkyl groups such as methyl, ethyl, n-propyl, and n-butyl. The preferred alkyl definition also includes C₃₋₅ branched alkyl groups, including CH(CH₃)₂, CH₂CH(CH₃)₂, CH(CH₃)CH₂CH₃, C(CH₃)₃, CH(CH₃)CH₂CH₂CH₃, CH(CH₃)CH(CH₃)₂, CH₂CH(CH₃)CH₂CH₃, CH₂CH₂CH(CH₃)₂, and CH(CH₂CH₃)₂.

The term “alkenyl” refers to alkyl groups as defined above, wherein there is at least one point of unsaturation, i.e., wherein two adjacent carbon atoms are attached by a double bond. The term “alkynyl” refers to alkyl groups wherein two adjacent carbon atoms are attached by a triple bond. The term ‘alkoxy” refers to —OR, wherein R is alkyl.

As used herein, the term “halogen” or “halo” refers to chloro, bromo, fluoro and iodo groups. “Haloalkyl” refers to an alkyl radical substituted with one or more halogen atoms. The term “haloalkyl” thus includes monohalo alkyl, dihalo alkyl, trihalo alkyl and the like. Representative monohalo alkyl groups include —CH₂F, —CH₂Cl, —CH₂CH₂F, —CH₂CH₂Cl, —CH(F)CH₃, —CH(Cl)CH₃; representative dihalo alkyl groups include CHCl₂, —CHF₂, —CCl₂CH₃, —CH(Cl)CH₂Cl, —CH₂CHCl₂, —CH₂CHF₂; representative trihalo alkyl groups include —CCl₃, —CF₃, —CCl₂CH₂Cl, —CF₂CH₂F, —CH(Cl)CHCl₂, —CH(F)CHF₂; and representative perhalo alkyl groups include —CCl₃, —CF₃, —CCl₂CCl₃, —CF₂CF₃.

“Amino” refers herein to the group —NH₂. The term “alkylamino” refers herein to the group —NRR′ where R and R′ are each independently selected from hydrogen or a lower alkyl. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl. The term cyano refers to the group —CN. The term nitro refers to the group —NO₂.

The term “alkoxyalkyl” refers to the group -alk₁-O-alk₂ where alk₁ is alkyl or alkenyl, and alk₂ is alkyl or alkenyl. The term “loweralkoxyalkyl” refers to an alkoxyalkyl where alk₁ is loweralkyl or loweralkenyl, and alk₂ is loweralkyl or loweralkenyl. The term “aryloxyalkyl” refers to the group -alkyl-O-aryl. The term “aralkoxyalkyl” refers to the group -alkylenyl-O-aralkyl, where aralkyl is a loweraralkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. In some embodiments, R and R′, together with the N atom attached to them may be taken together to form a “heterocycloalkylcarbonyl” group. The term “arylaminocarbonyl” refers herein to the group —C(O)—NRR′ where R is an aryl and R′ is hydrogen, loweralkyl or aryl. “aralkylaminocarbonyl” refers herein to the group —C(O)—NRR′ where R is loweraralkyl and R′ is hydrogen, loweralkyl, aryl, or loweraralkyl.

“Carbonyl” refers to the divalent group —C(O)—. “Carboxy” refers to —C(═O)—OH. “Alkoxycarbonyl” refers to ester —C(═O)—OR wherein R is alkyl. “Loweralkoxycarbonyl” refers to ester —C(═O)—OR wherein R is loweralkyl. “Cycloalkyloxycarbonyl” refers to —C(═O)—OR wherein R is cycloalkyl.

“Cycloalkyl” refers to a mono- or polycyclic, carbocyclic alkyl substituent. Carbocycloalkyl groups are cycloalkyl groups in which all ring atoms are carbon. Typical cycloalkyl substituents have from 3 to 8 backbone (i.e., ring) atoms in which each backbone atom is either carbon or a heteroatom. The term “heterocycloalkyl” refers herein to cycloalkyl substituents that have from 1 to 5, and more typically from 1 to 4 heteroatoms in the ring structure. Suitable heteroatoms employed in compounds of the present invention are nitrogen, oxygen, and sulfur. Representative heterocycloalkyl moieties include, for example, morpholino, piperazinyl, piperidinyl and the like. Carbocycloalkyl groups are cycloalkyl groups in which all ring atoms are carbon. When used in connection with cycloalkyl substituents, the term “polycyclic” refers herein to fused and non-fused alkyl cyclic structures. The term “partially unsaturated cycloalkyl”, “partially saturated cycloalkyl”, and “cycloalkenyl” all refer to a cycloalkyl group wherein there is at least one point of unsaturation, i.e., wherein to adjacent ring atoms are connected by a double bond or a triple bond. Illustrative examples include cyclohexynyl, cyclohexynyl, cyclopropenyl, cyclobutynyl, and the like.

The terms “substituted heterocycle”, “heterocyclic group” or “heterocycle” as used herein refers to any 3- or 4-membered ring containing at least one oxygen atom and the other heteroatoms selected from nitrogen, oxygen, and sulfur or a 5- or 6-membered ring containing at least one oxygen atom and the remaining optional two heteroatoms selected from the group consisting of nitrogen, oxygen, or sulfur; wherein the 5-membered ring has 0-2 double bonds and the 6-membered ring has 0-3 double bonds; wherein the nitrogen and sulfur atom maybe optionally oxidized; wherein the nitrogen and sulfur heteroatoms may be optionally quaternized; and including any bicyclic group in which any of the above heterocyclic rings is fused to a benzene ring or another 5- or 6-membered heterocyclic ring independently defined above. The term or “heterocycloalkyl” as used herein refers to a 5- or 6-membered ring containing from one to three heteroatoms selected from the group consisting of nitrogen, oxygen, or sulfur, wherein the ring has no double bonds. For example, the term heterocyclo-C₅-alkyl refers to a 6-membered ring containing 5 carbon atoms and a heteroatom, such as N. The term “heterocycle” thus includes rings in which nitrogen is the heteroatom as well as partially and fully-saturated rings. Preferred heterocycles include, for example: diazapinyl, pyrryl, pyrrolinyl, pyrrolidinyl, pyrazolyl, pyrazolinyl, pyrazolidinyl, imidazolyl, imidazolinyl, imidazolidinyl, pyridyl, piperidinyl, pyrazinyl, piperazinyl, N-methyl piperazinyl, azetidinyl, N-methylazetidinyl, pyrimidinyl, pyridazinyl, oxazolyl, oxazolidinyl, isoxazolyl, isoazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl and benzothienyl. The foregoing list will be changed bases on the above changes.

Heterocyclic moieties can be unsubstituted or monosubstituted or disubstituted or trisubstituted with various substituents independently selected from hydroxy, halo, oxo (C═O), alkylimino (RN═, wherein R is a loweralkyl or loweralkoxy group), amino, alkylamino, dialkylamino, acylaminoalkyl, alkoxy, thioalkoxy, polyalkoxy, loweralkyl, cycloalkyl or haloalkyl.

The heterocyclic groups may be attached at various positions as will be apparent to those having skill in the organic and medicinal chemistry arts in conjunction with the disclosure herein.

Representative heterocyclics include, for example, imidazolyl, pyridyl, piperazinyl, piperidinyl, azetidinyl, thiazolyl, furanyl, triazolyl benzimidazolyl, benzothiazolyl, benzoxazolyl, quinolinyl, isoquinolinyl, quinazolinyl, quinoxalinyl, phthalazinyl, indolyl, naphthpyridinyl, indazolyl, and quinolizinyl.

“Aryl” refers to optionally substituted monocyclic and polycyclic aromatic groups having from 3 to 14 backbone carbon or hetero atoms, and includes both carbocyclic aryl groups and heterocyclic aryl groups. Carbocyclic aryl groups are aryl groups in which all ring atoms in the aromatic ring are carbon. The term “heteroaryl” refers herein to aryl groups having from 1 to 4 heteroatoms as ring atoms in an aromatic ring with the remainder of the ring atoms being carbon atoms. When used in connection with aryl substituents, the term “polycyclic aryl” refers herein to fused and non-fused cyclic structures in which at least one cyclic structure is aromatic, such as, for example, benzodioxozolo (which has a heterocyclic structure fused to a phenyl group, i.e., and the like. Exemplary aryl moieties employed as substituents in compounds of the present invention include phenyl, pyridyl, pyrimidinyl, thiazolyl, indolyl, imidazolyl, oxadiazolyl, tetrazolyl, pyrazinyl, triazolyl, thiophenyl, furanyl, quinolinyl, purinyl, naphthyl, benzothiazolyl, benzopyridyl, and benzimidazolyl, and the like.

“Optionally substituted” or “substituted” refers to the replacement of one or more hydrogen atoms with a monovalent or divalent radical. Suitable substitution groups include, for example, hydroxy, nitro, amino, imino, cyano, halo, thio, sulfonyl, thioamido, amidino, imidino, oxo, oxamidino, methoxamidino, imidino, guanidino, sulfonamido, carboxyl, formyl, loweralkyl, haloloweralkyl, loweralkylamino, haloloweralkylamino, loweralkoxy, haloloweralkoxy, loweralkoxyalkyl, alkylcarbonyl, aminocarbonyl, arylcarbonyl, aralkylcarbonyl, heteroarylcarbonyl, heteroaralkylcarbonyl, alkylthio, aminoalkyl, cyanoalkyl, aryl and the like.

The substitution group can itself be substituted. The group substituted onto the substitution group can be carboxyl, halo; nitro, amino, cyano, hydroxy, loweralkyl, loweralkoxy, aminocarbonyl, —SR, thioamido, —SO₃H, —SO₂R or cycloalkyl, where R is typically hydrogen, hydroxyl or loweralkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms. It is understood that the above definitions are not intended to include impermissible substitution patterns (e.g., methyl substituted with five fluoro groups or a halogen atom substituted with another halogen atom). Such impermissible substitution patterns are well known to the skilled artisan.

It will also be apparent to those skilled in the art that the compounds of the invention, or their stereoisomers, as well as the pharmaceutically acceptable salts, esters, metabolites and prodrugs of any of them, may be subject to tautomerization and may therefore exist in various tautomeric forms wherein a proton of one atom of a molecule shifts to another atom and the chemical bonds between the atoms of the molecules are consequently rearranged. See, e.g., March, Advanced Organic Chemistry: Reactions, Mechanisms and Structures, Fourth Edition, John Wiley & Sons, pages 69-74 (1992). As used herein, the term “tautomer” refers to the compounds produced by the proton shift, and it should be understood that the all tautomeric forms, insofar as they may exist, are included within the invention.

The compounds of the invention, or their tautomers, as well as the pharmaceutically acceptable salts, esters, metabolites and prodrugs of any of them, may comprise asymmetrically substituted carbon atoms. Such asymmetrically substituted carbon atoms can result in the compounds of the invention existing in enantiomers, diastereomers, and other stereoisomeric forms that may be defined, in terms of absolute stereochemistry, such as in (R)— or (S)— forms. As a result, all such possible isomers, individual stereoisomers in their optically pure forms, mixtures thereof, racemic mixtures (or “racemates”), mixtures of diastereomers, as well as single diastereomers of the compounds of the invention are included in the present invention. The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

As used herein, the term “pharmaceutically acceptable salts” refers to the nontoxic acid or alkaline earth metal salts of the compounds of Formula I. These salts can be prepared in situ during the final isolation and purification of the compounds of Formula I or II, or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

It will be apparent to those skilled in the art that the compounds of the invention, or their tautomers, prodrugs and stereoisomers, as well as the pharmaceutically acceptable salts, esters and prodrugs of any of them, may be processed in vivo through metabolism in a human or animal body or cell to produce metabolites. The term “metabolite” as used herein refers to the formula of any derivative produced in a subject after administration of a parent compound. The derivatives may be produced from the parent compound by various biochemical transformations in the subject such as, for example, oxidation, reduction, hydrolysis, or conjugation and include, for example, oxides and demethylated derivatives. The metabolites of a compound of the invention may be identified using routine techniques known in the art. See, e.g., Bertolini, G. et al., J. Med. Chem. 40:2011-2016 (1997); Shan, D. et al., J. Pharm. Sci. 86(7):765-767; Bagshawe K., Drug Dev. Res. 34:220-230 (1995); Bodor, N., Advances in Drug Res. 13:224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991). It should be understood that individual chemical compounds that are metabolites of the compounds of formula I, formula II, or their tautomers, prodrugs and stereoisomers, as well as the pharmaceutically acceptable salts, esters and prodrugs of any of them, are included within the invention.

The term “cancer” refers to cancer diseases that can be beneficially treated by the inhibition of Pim kinase, including, for example, solid cancers, such as carcinomas (e.g., of the lungs, pancreas, thyroid, ovarian, bladder, breast, prostate, or colon), melanomas, myeloid disorders (e.g., myeloid leukemia, multiple myeloma and erythroleukemia), adenomas (e.g., villous colon adenoma) and sarcomas (e.g., osteosarcoma).

### Synthetic Methods

Compounds of the invention can be obtained through procedures known to the skilled in the art. For example, as shown in Scheme 1, D-glucal can be protected as the tris-triisopropylsilyl (TIPS) compound (R₁₁ and R₁₂=OTIPS) which upon lithiation and quench with trimethyl borate yields the trisTIPS-D-glucal boronic acid I. Subsequent Suzuki reaction with nitro aryl or nitroheteroaryl halides, such as 4-chloro, 3-nitro pyridine, yields C₂ carbon modified glucal II. The least hindered primary TIPS group can be deprotected selectively and modified via the resulting primary hydroxyl or oxidized aldehyde III, to introduce a range of groups (R₁₄) at the C₆ glucal position. Subsequent nitro or nitro & alkene reduction, acid coupling and removal of protecting groups yield compounds of the invention IV. In compounds such as IV, if R₁₈ is halo or triflate, compounds such as IV can be further modified by standard methods to introduce substituted aryls, alkyls and heteroaryls at R₁₈. For example, if R₁₈ is Br, by reaction with boronic acids or organometallic reagents, or conversion to the corresponding boronate ester and reaction with aryl/heteroaryl halides or triflates, a variety of R₁₈ modifications are possible.

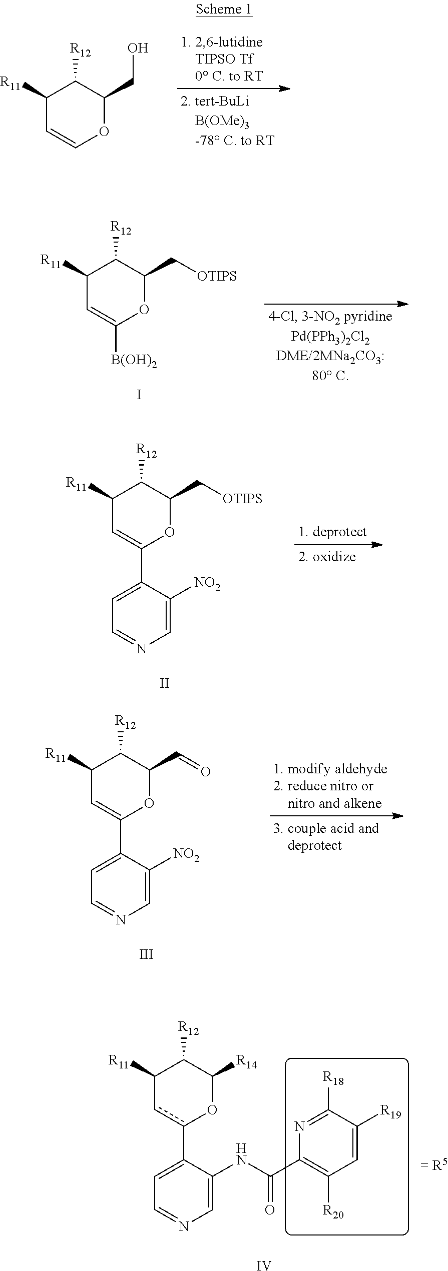

Alternatively, as shown in Scheme 2, compounds of the invention can be obtained following a hetero-Diels Alder construction of pyran rings. Reaction of nitroaryl aldehydes or nitroheteroaryl aldehydes such as 3-nitro, isonicotinaldehyde (R₇=H), with alkoxysubstituted dienes (i.e. R₁₁=OTES) yields pyran enol silanes V which can be oxidized to yield polysubstituted hydroxypyranones (R₁₃=OH) or directly hydrolyzed to yield polysubstituted pyranones (R₁₃=H) in which the R₈, R₉, R₁₁, R₁₂, R₁₄, R₁₅ and heteroaryl groups are derived from the diene and aldehyde substituents. Reduction of the pyranone carbonyl (R₁₀=H), hydroxylprotection and nitro reduction yields heteroarylaniline VII. Alternatively, as shown in Scheme 2a, reductive amination of the pyranone carbonyl, debenzylation and nitro reduction followed by protection with the Boc group yields heteroarylaniline VIIa (R₁₀=H, R₁₁=NHBoc).

Subsequent coupling of VII or VIIa with heterocyclic acids (i.e. R₅CO₂H) and deprotection of protecting groups yields compounds of the invention VIII and VIIIa. Subsequent coupling with heterocyclic acids (i.e. R₅CO₂H) and deprotection of protecting groups yields compounds of the invention VIII. In compounds such as VIII, if R₁₈ is halo or triflate, compounds such as VIII can be further modified by standard methods to introduce substituted aryls, alkyls and heteroaryls at R₁₈. For example, if R₁₈ is Br, by reaction with boronic acids or organometallic reagents, or conversion to the corresponding boronate ester and reaction with aryl/heteroaryl halides or triflates, a variety of R₁₈ modifications are possible.

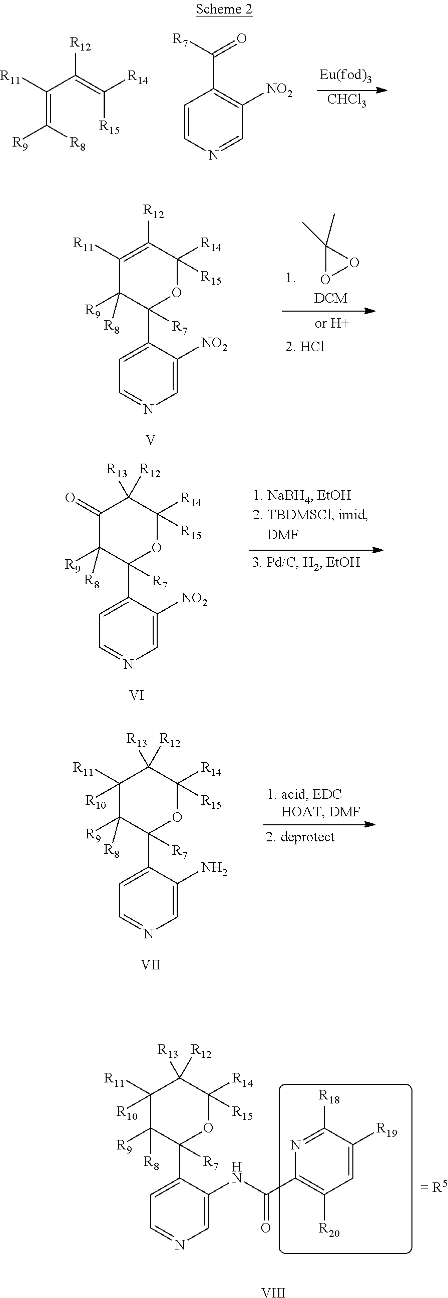

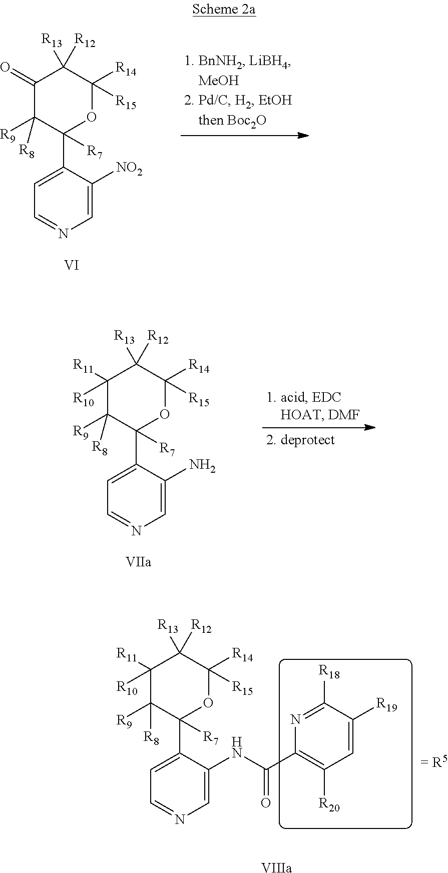

The enol silane V is a versatile intermediate for which to introduce substituents at the pyran C₃ position, as indicated in Scheme 3, where reaction of the enol silane V (where R₁₁=OSiR₃ and R₁₂=H) with Eschenmosher's salt, and subsequent methylation, elimination and ketone reduction yields exocyclic pyran alkene IX. Modification of the alkene via electrophilic means (dihydroxylation and subsequent diol modification or epoxidation and subsequent nucleophilic epoxide opening for example) as well as oxidation to the ketone and subsequent nucleophilic modification are among the possible manipulations of enol silane V to introduce substitutions (R₁₂ and R₁₃ in Scheme 3) at the C₃ position of the pyran. After alkene modification, nitro reduction, acid coupling and protecting group deprotection yields compounds of the invention X. In compounds such as X, if R₁₈ is halo or triflate, compounds such as X can be further modified by standard methods to introduce substituted aryls, alkyls and heteroaryls at R₁₈. For example, if R₁₈ is Br, by reaction with boronic acids or organometallic reagents, or conversion to the corresponding boronate ester and reaction with aryl/heteroaryl halides or triflates, a variety of R₁₈ modifications are possible.

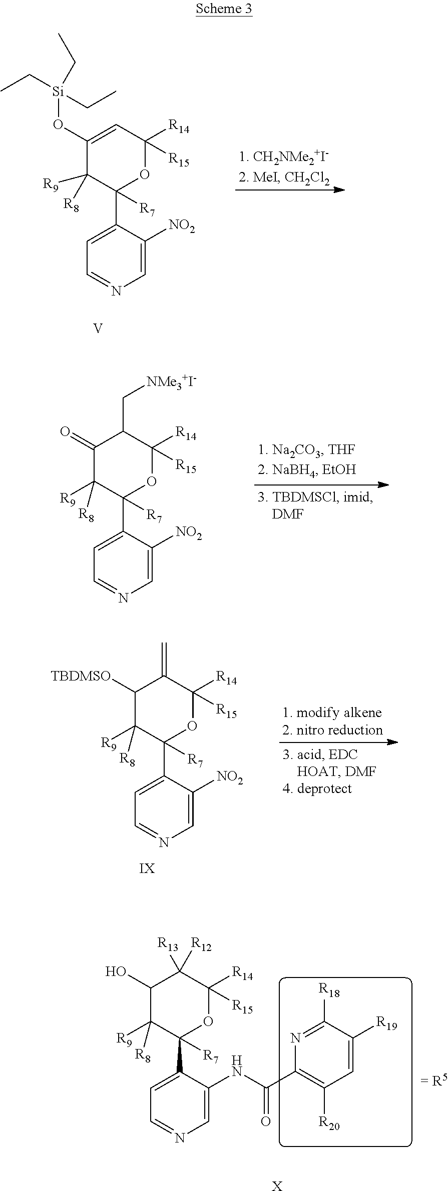

Alternatively as shown in Scheme 4, cyclic ketal nitroarenes X¹ can be obtained by condensation of diols and nitroaryl aldehydes or nitroheteroarylaldehydes, such as 3-nitro isonicotinicaldehyde. Subsequent nitro reduction yields aniline XII which can be coupled to heterocyclic acids that upon protecting group removal yield compounds of the invention XIII. In compounds such as XIII, if R₁₈ is halo or triflate, compounds such as XIII can be further modified by standard modifications to introduce substituted aryls, alkyls and heteroaryls at R₁₈. For example, if R₁₈ is Br, by reaction with boronic acids or organometallic reagents, or conversion to the corresponding boronate ester and reaction with aryl/heteroaryl halides or triflates, a variety of R₁₈ modifications are possible.

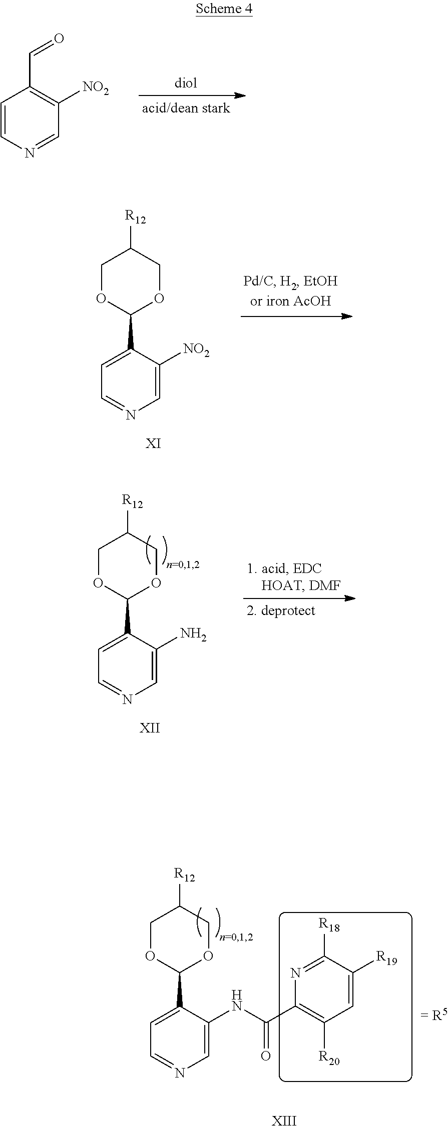

### EXAMPLES

Referring to the examples that follow, compounds of the preferred embodiments can be synthesized using the methods described herein, or other methods, which are known in the art.

The compounds and/or intermediates were characterized by high performance liquid chromatography (HPLC) on one of two instruments: a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from EMD Chemicals Inc; another instrument was a Waters system (ACQUITY UPLC system; column ACQUITY UPLC HSS-C18, 1.8 um, 2.1×50 mm; gradient: 5-95% acetonitrile in water with 0.05% TFA over 2 min or 10 min period; flow rate 1.2 mL/min; column temperature 50° C.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well-known iodine vapor and other various staining techniques.

Mass spectrometric analysis was performed on Waters System (ACQUITY UPLC system and a ZQ 2000 system; Column: ACQUITY UPLC HSS-C18, 1.8 um, 2.1×50 mm; gradient: 5-95% (or 35-95%, or 65-95% or 95-95%) acetonitrile in water with 0.05% TFA over a 1.5 min period; flow rate 1.2 mL/min; molecular weight range 150-850; cone Voltage 20 V; column temperature 50° C.). All masses were reported as those of the protonated parent ions.

Nuclear magnetic resonance (NMR) analysis was performed on some of the compounds with a Varian 400 or 300 MHz NMR (Palo Alto, Calif.). The spectral reference was either TMS or the known chemical shift of the solvent.

Preparative separations are carried out using an ISCO or Analogix automated silica gel chromatography systems Flash 40 chromatography system and KP-Sil, 60A (Biotage, Charlottesville, Va.), or by flash column chromatography using silica gel (230-400 mesh) packing material, or by HPLC using a Waters 2767 Sample Manager, Waters Sunfire Prep C-18 reversed phase column, 5 um. Typical solvents employed for the ISCO or Analogix systems and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

Preparative separation of enantiomers was carried out using Waters Delta Prep system. Chiral columns are selected among AD, AS, OD, OJ, IA and IC (Chiral Technologies Inc. West Chester, Pa.). The eluting solvents are either heptane/EtOH or heptane/IPA.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings

| | ABBREVIATIONS |
|---|---|
| DAST | (diethylamino)sulfurtrifluoride |
| DCM | Dichloromethane |
| DIEA | diisopropylethylamine |
| DMA | Dimethylacetamide |
| DMAP | 4-dimethylaminopyridine |
| DMDO | Dimethyl dioxirane |
| DME | 1,2-dimethoxyethane |
| DMF | N,N-dimethylformamide |
| DMSO | Dimethyl sulfoxide |
| DPPF | 1,1′-bis(diphenylphosphino)ferrocene |
| EDC | 1-(3-Dimethylaminopropyl)-3- |
| | ethylcarbodiimide hydrochloride |
| EtOAc | ethyl acetate |
| EtOH | Ethanol |
| Eu(fod)₃ | tris(6,6,7,7,8,8,8-heptafluoro-2,2-dimethyl- |
| | 3,5-octanedionato) europium |
| HOAT | Hydroxyazabenzotriazole |
| K₂CO₃ | Potassium carbonate |
| MeCN | Acetonitrile |
| MgSO₄ | Magnesium sulfate |
| MeOH | Methanol |
| Na₂CO₃ | sodium carbonate |
| NaCl | Sodium chloride |
| NaHCO₃ | sodium bicarbonate |
| Na2CO3 | Sodium carbonate |
| NBS | N-bromosuccinimide |
| NMP | N-methyl-2-pyrrolidone |
| Pd₂(dba)₃ | Tris(dibenzylideneacetone)dipalladium(0) |
| Pd(PPh₃)₄ | Tetrakis(triphenylphospine)palladium(0) |
| Pd(dppf)Cl₂- | Dichloro-(1,2-bis(diphenylphosphino)ethan)- |
| DCM | Palladium(II)-dichloromethane adduct |
| RT or rt | room temperature |
| TBDMSC1 | tert-butyldimethylsilylchloride |
| TBAF | Tetrabutylammonium fluoride |
| TEA | Triethylamine |
| THF | tetrahydrofuran |
| TFA | Trifluoroacetic acid |

### Synthesis of 2,6-difluorobenzothioamide

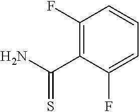 [SMILES: NC(=S)c1c(F)cccc1F]

A solution of 2, 6 difluorobenzamide (1 eq) and Lawesson's reagent (0.5 eq.) in toluene (0.2 M) was heated at 90° C. for 14 hours. Upon cooling the volatiles were removed in vacuo and purified by SiO₂ chromatography (25% EtOAc/hexanes) yielding 2,6-difluorobenzothioamide as a light yellow solid (99%). LCMS (m/z): 174.1 (MH⁺); LC Rₜ=2.19 min.

### Synthesis of ethyl 2-(2,6-difluorophenyl)thiazole-4-carboxylate

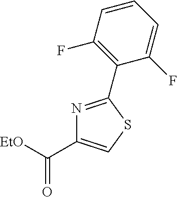 [SMILES: CCOC(=O)c1csc(-c2c(F)cccc2F)n1]

A solution of 2,6-difluorobenzothioamide (1.0 eq) and ethylbromopyruvate (1.0 eq.) in ethanol (1.0 M) was heated in the microwave at 130° C. for 30 minutes. Upon removal of volatiles in vacuo, ethyl acetate was added and the solution was washed with Na₂CO_{3(sat.)}, with NaCl_{(sat.)}, was dried over MgSO₄, filtered and concentrated yielding ethyl 2-(2,6-difluorophenyl)thiazole-4-carboxylate (84%). LCMS (m/z): 270.1 (MH⁺); LC Rₜ=3.79 min.

### Synthesis of 2-(2,6-difluorophenyl)thiazole-4-carboxylic acid

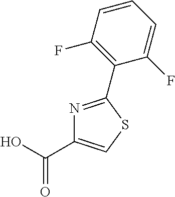 [SMILES: O=C(O)c1csc(-c2c(F)cccc2F)n1]

To a solution of ethyl 2-(2,6-difluorophenyl)thiazole-4-carboxylate (1.0 eq.) in 2:1 THF/MeOH (0.17 M) was added 1.0 M LiOH (2.0 eq.). After standing for 16 hours, 1.0 M HCl (2.0 eq.) was added and the THF/MeOH was removed in vacuo. The resulting solid was filtered, rinsed with H₂O and dried, yielding 2-(2,6-difluorophenyl)thiazole-4-carboxylic acid (88%) as a crusty solid. LCMS (m/z): 251.1 (MH⁺); LC Rₜ=2.68 min.

### Synthesis of ethyl 2-amino-2-cyanoacetate

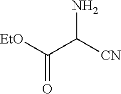

To a solution of ethyl 2-cyano-2-(hydroxyimino)acetate (1 eq) in 70 mL of water and 56 mL of aq. sat. sodium bicarbonate was added portionwise throughout 10 minutes Na₂S₂O₄ (2.8 eq) The reaction mixture was stirred at room temperature for 1 hour. The solution was saturated with sodium chloride, extracted with methylene chloride (300 mL×3) and then the combined organic layers were dried over anhydrous Na₂SO₄, filtered, and concentrated in vacuo to give the titled compound, which was used to next step without further (55%). LC/MS (m/z): 129.0 (MH⁺), Rₜ: 0.25 min.

### Synthesis of ethyl 2-cyano-2-(2,6-difluorobenzamido)acetate

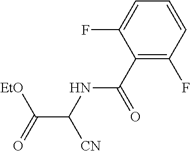 [SMILES: CCOC(=O)C(C#N)NC(=O)c1c(F)cccc1F]

To a solution of ethyl 2-amino-2-cyanoacetate (1 eq) in 6 mL of dichloromethane was added pyridine (1.5 eq) and 2,6-difluorobenzoyl chloride (1 eq) at 0° C. The reaction mixture was stirred at room temperature for 3 hours. The mixture was diluted with ethyl acetate, washed with brine, then dried over anhydrous MgSO₄, filtered, and concentrated in vacuo. The crude residue was purified by flash chromatography (EtOAc:hexanes=1:1) to give the titled compound (84%). LC/MS (m/z): 269.1 (MH⁺), Rₜ: 0.69 min.

### Synthesis of 5-amino-2-(2,6-difluorophenyl)thiazole-4-carboxylic acid

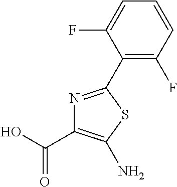 [SMILES: Nc1sc(-c2c(F)cccc2F)nc1C(=O)O]

To a solution of the ethyl 2-cyano-2-(2,6-difluorobenzamido)acetate (1 eq) in 10 mL of toluene was added Lawesson reagent. The mixture was stirred at 95° C. for 2 days. Solvents were removed under reduced pressure. The crude residue was purified by flash chromatography (EtOAc:hexanes=1:1) to give the ethyl 5-amino-2-(2,6-difluorophenyl)thiazole-4-carboxylate, which was dissolved in 5 mL of methanol and 5 mL of THF. Then the mixture was added 1M sodium hydroxide (2 eq). The reaction mixture was stirred at room temperature overnight. The reaction was concentrated to remove most of solvents. The residue was extracted with ethyl acetate. The aqueous layer was acidified to pH=4-5 by 1N HCl. The resulting mixture was extracted by ethyl acetate. The organic layer was separated, washed with brine, then dried over anhydrous MgSO₄, filtered, and concentrated in vacuo to give the pure titled compound (34%). LC/MS (m/z): 257.1 (MH⁺), Rₜ: 0.61 min.

### Synthesis of 6-bromo-5-fluoropicolinic acid

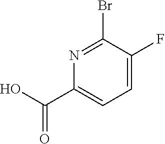 [SMILES: O=C(O)c1ccc(F)c(Br)n1]

To 2-bromo-3-fluoro-6-methylpyridine (1.0 equiv.) in H₂O (30 mL) was added potassium permanganate (1.0 equiv.). The solution was heated at 100° C. for 5 hours at which time more potassium permanganate (1.0 equiv.) was added. After heating for an additional 48 hours the material was filtered through celite (4 cm×2 inches) and rinsed with H₂O (150 mL). The combined aqueous was acidified with 1N HCl to pH=4, extracted with ethyl acetate (200 mL), washed with NaCl_{(sat.)}, dried over MgSO₄, filtered and concentrated to yield 6-bromo-5-fluoropicolinic acid (17%) as a white solid. LCMS (m/z): 221.9 (MH⁺); LC Rₜ=2.05 min.

### Synthesis of 2-chloro-6-phenylpyrazine

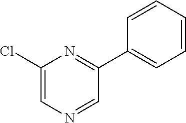 [SMILES: Clc1cncc(-c2ccccc2)n1]

To a solution of 2,6-dichloropyrazine (2.0 equiv.) in 3:1 DME: 2M aqueous sodium carbonate (0.125 M) was added phenylboronic acid (1.0 equiv.) then PdCl₂(dppf).DCM adduct (0.1 equiv.). The reaction was heated in the microwave at 120° C. for 15 minutes. The crude reaction mixture was diluted with ethyl acetate and washed with sat. aq. sodium bicarbonate then sat. NaCl. The organic phase was dried with magnesium sulfate, filtered, and concentrated. The crude material was purified by silica gel column chromatography with heptanes to 30% ethyl acetate in heptanes to give 2-chloro-6-phenylpyrazine in 75% yield. LC/MS (m/z): 191.0 (MH+) Rₜ=1.00 min.

### Synthesis of methyl 6-phenylpyrazine-2-carboxylate

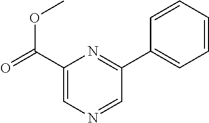 [SMILES: COC(=O)c1cncc(-c2ccccc2)n1]

To a steel pressure vessel with a stir bar was added a solution of 2-chloro-6-phenylpyrazine (1 equiv.) in MeOH (0.2 M) followed by triethylamine (1.5 equiv.) which was degassed with nitrogen for 5 min. DIEA (2.5 equiv.) was added. Pd (II) R-Binap (0.012 equiv.) was added then the reaction vessel was sealed and then carbon monoxide atmosphere was added to 70 psi. The mixture was then heated to 100° C. for 18 hours. The reaction mixture was diluted with ethyl acetate and washed with water then sat. NaCl. The organic phase was dried with sodium sulfate, filtered, and concentrated. The crude material was purified by silica gel column chromatography with heptanes to 20% ethyl acetate in heptanes to give 6-phenylpyrazine-2-carboxylate in 99% yield. LC/MS (m/z): 215.0 (MH⁺), Rₜ=0.73 min.

### Synthesis of 6-phenylpyrazine-2-carboxylic acid

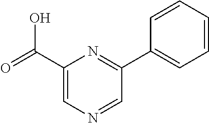 [SMILES: O=C(O)c1cncc(-c2ccccc2)n1]

To a solution of 6-phenylpyrazine-2-carboxylate (1.0 equiv.) in THF (0.2 M) was added a 2 M solution of LiOH (10 equiv.) and allowed to stir over two days at rt. The reaction mixture was acidified with 1N HCl until a white solid precipitated and then filtered. The solid was dried overnight on the high-vac to remove all water to yield 6-phenylpyrazine-2-carboxylic acid in 67% yield. LC/MS (m/z): 201.0 (MH⁺), Rₜ=0.62 min.

### Synthesis of Methyl 3-amino-5-fluoropicolinate

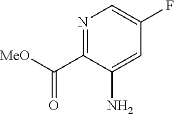 [SMILES: COC(=O)c1ncc(F)cc1N]

To a steel bomb reactor, 2-bromo-5-fluoropyridin-3-amine (1.0 equiv.), triethylamine (1.6 equiv.), Pd(BINAP)Cl₂ (0.0015 equiv.) and anhydrous methanol (0.4 M solution) were added. After degassed by nitrogen stream for 15 min, the steel bomb reactor was closed and filled with CO gas up to 60 psi. The reactor was then heated to 100° C. After 3 h, more Pd catalyst (0.0015 equiv.) was added and the reaction mixture was re-heated to the same temperature for 3 h. After cooling down to room temperature, a brown precipitate was filtered off and the filtrate was extracted with EtOAc, which was washed with water and brine, dried over anhydrous sodium sulfate, and filtered. After removing volatile materials, the crude yellow product was obtained and used for the next step without further purification (40%). LCMS (m/z): 271.2 (MH⁺); LC Rₜ=3.56 min.

### Synthesis of Methyl 3-amino-6-bromo-5-fluoropicolinate

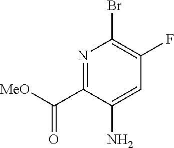 [SMILES: COC(=O)c1nc(Br)c(F)cc1N]

To a solution of methyl 3-amino-5-fluoropicolinate (1.0 equiv.) in acetonitrile (0.3 M solution) was added NBS (1.1 equiv.) for 2 minutes at room temperature. After quenched with water, the reaction mixture was extracted with EtOAc. The crude product was purified by silica column chromatography (20% to 50% EtOAc in hexanes) to give methyl 3-amino-6-bromo-5-fluoropicolinate, (41%). LCMS (m/z): 249.1 (MH⁺); LC Rₜ=2.80 min.

### Synthesis of 6-bromo-5-fluoropicolinic acid

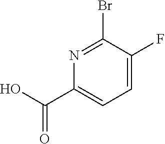 [SMILES: O=C(O)c1ccc(F)c(Br)n1]

To 2-bromo-3-fluoro-6-methylpyridine (1.0 equiv.) in H₂O (30 mL) was added potassium permanganate (1.0 equiv.). The solution was heated at 100° C. for 5 hours at which time more potassium permanganate (1.0 equiv.) was added. After heating for an additional 48 hours the material was filtered through celite (4 cm×2 inches) and rinsed with H₂O (150 mL). The combined aqueous was acidified with 1N HCl to pH=4, extracted with ethyl acetate (200 mL), washed with NaCl(sat.), dried over MgSO₄, filtered and concentrated to yield 6-bromo-5-fluoropicolinic acid (17%) as a white solid. LCMS (m/z): 221.9 (MH+); LC Rt=2.05 min.

### Synthesis of methyl 6-bromo-5-fluoropicolinate

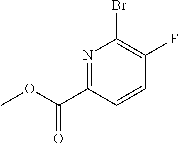

To a solution of 6-bromo-5-fluoropicolinic acid (1.0 equiv.) in methanol (0.2 M) was added H₂SO₄ (4.2 equiv.) and the reaction was stirred at room temperature for two hours. Upon completion of the reaction as monitored by LC/MS, the reaction was diluted with ethyl acetate and quenched slowly with saturated aqueous NaHCO₃. The reaction was poured into a separatory funnel and extracted with ethyl acetate. The organic phase was dried with magnesium sulfate, filtered, and concentrated in vacuo to provide methyl 6-bromo-5-fluoropicolinate as a white solid (>99%). LCMS (m/z): 233.9/235.9 (MH), Rₜ=0.69 min.

### Method 1

### Synthesis of methyl 3-amino-6-(2,6-difluorophenyl)picolinate

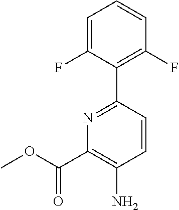 [SMILES: COC(=O)c1nc(-c2c(F)cccc2F)ccc1N]

A solution of methyl 3-amino-6-bromopicolinate (1.0 equiv.), 2,6-difluorophenyl-boronic acid (3.0 equiv), and Pd(dppf)Cl₂-DCM (0.1 equiv.) in 3:1 DME/2M Na₂CO₃ (0.5 M) was subjected to microwave irradiation at 120° C. for 15 min intervals. The reaction was filtered and washed with EtOAc. The organic was partitioned with H₂O (25 mL), was further washed with NaCl_{(sat.)} (25 mL), was dried over MgSO₄, and the volatiles were removed in vacuo. The residue was diluted in EtOAc and passed through a silica gel plug and the volatiles were removed in vacuo yielding methyl 3-amino-6-(2,6-difluorophenyl)picolinate (47%). LCMS (m/z): 265.1 (MH⁺); LC Rₜ=2.70 min.

### Method 2

### Synthesis of 3-amino-6-(2,6-difluorophenyl)picolinic acid

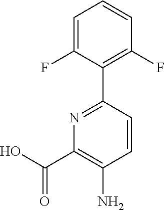 [SMILES: Nc1ccc(-c2c(F)cccc2F)nc1C(=O)O]

To a solution of methyl 3-amino-6-(2,6-difluorophenyl)picolinate (1.0 equiv) in THF (0.5 M), was added 1M LiOH (4.0 equiv). After stirring for 4 hours at 60° C., 1 N HCl (4.0 equiv.) was added and the THF was removed in vacuo. The resulting solid was filtered and rinsed with cold H₂O (3×20 mL) to yield 3-amino-6-(2,6-difluorophenyl)picolinic acid (90%). LCMS (m/z): 251.1 (MH⁺); LC Rₜ=2.1 min.

### Synthesis of methyl 3-amino-5-fluoro-6-(2-fluorophenyl)picolinate

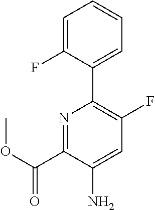 [SMILES: COC(=O)c1nc(-c2ccccc2F)c(F)cc1N]

Method 1 was followed using methyl 3-amino-6-bromo-5-fluoropicolinate (1.0 equiv.) and 2-fluoro-phenylboronic acid (1.5 equiv.) and Pd(dppf)Cl₂-DCM (0.05 equiv.) to give methyl 3-amino-5-fluoro-6-(2-fluorophenyl)picolinate in >99% yield. LCMS (m/z): 265.0 (MH⁺), Rₜ=0.77 min.

### Synthesis of 3-amino-5-fluoro-6-(2-fluorophenyl)picolinic acid

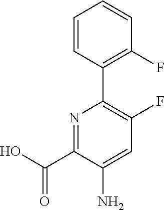 [SMILES: Nc1cc(F)c(-c2ccccc2F)nc1C(=O)O]

Method 2 was followed using 3-amino-5-fluoro-6-(2-fluorophenyl)picolinate (1.0 equiv.) and LiOH (5.0 equiv.) to give 3-amino-5-fluoro-6-(2-fluorophenyl)picolinic acid in 90% yield. LCMS (m/z): 251.1 (MH⁺), Rₜ=0.80 min.

### Synthesis of 6-(2-fluoro-5-(isopropylcarbamoyl)phenyl)picolinic acid

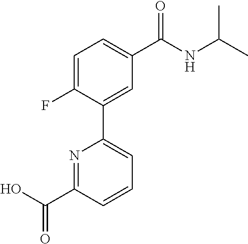 [SMILES: CC(C)NC(=O)c1ccc(F)c(-c2cccc(C(=O)O)n2)c1]

Method 1 and 2 were followed using methyl 6-bromopicolinate (1.0 equiv.) and 2-fluoro-5-(isopropylcarbamoyl)phenylboronic acid (1.5 equiv.) and Pd(dppf)Cl₂-DCM (0.1 equiv.) to give 6-(2-fluoro-5-(isopropylcarbamoyl)phenyl)picolinic acid. LCMS (m/z): 303.0 (MH⁺), Rₜ=0.65 min.

### Synthesis of 3-amino-6-phenylpyrazine-2-carboxylic acid

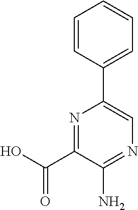 [SMILES: Nc1ncc(-c2ccccc2)nc1C(=O)O]

Method 1 and 2 were followed using methyl 3-amino-6-bromopyrazine-2-carboxylate (1.0 equiv.) and phenylboronic acid (2.0 equiv.) and Pd(dppf)Cl₂-DCM (0.05 equiv.) to give 3-amino-6-phenylpyrazine-2-carboxylic acid in 70% yield over the two steps. LCMS (m/z): 216.0 (MH⁺), Rₜ=0.67 min.

### Synthesis of methyl 3-amino-6-(2,6-difluorophenyl)-5-fluoropicolinate

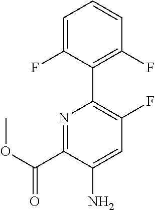 [SMILES: COC(=O)c1nc(-c2c(F)cccc2F)c(F)cc1N]

Method 1 was followed using methyl 3-amino-6-bromo-5-fluoro-picolinate (1.0 equiv.) and 2,6-difluorophenylboronic acid (1.3 equiv.) and Pd(dppf)Cl₂-DCM (0.05 equiv.) to give 3-amino-6-(2,6-difluorophenyl)-5-fluoropicolinate in 94% yield. LCMS (m/z): 283.0 (MH⁺), Rₜ=0.76 min.

### Synthesis of 3-amino-6-(2,6-difluorophenyl)-5-fluoropicolinic acid

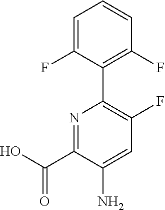 [SMILES: Nc1cc(F)c(-c2c(F)cccc2F)nc1C(=O)O]

Method 2 was followed using 3-amino-6-(2,6-difluorophenyl)-5-fluoropicolinate (1.0 equiv.) and LiOH (1.0 equiv.) to give 3-amino-6-(2,6-difluorophenyl)-5-fluoropicolinic acid in 79% yield. LCMS (m/z): 269.0 (MH⁺), Rₜ=0.79 min.

### Synthesis of methyl 3-amino-6-(2-fluoro-5-isopropylcabamoyl)phenyl)-picolinate

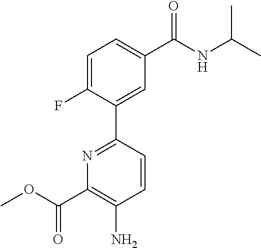 [SMILES: COC(=O)c1nc(-c2cc(C(=O)NC(C)C)ccc2F)ccc1N]

A solution of methyl 3-amino-6-bromopicolinate (1.0 equiv.), N-isopropyl 3-borono-4-fluorobenzamide (1.1 equiv.), and Pd(dppf)Cl₂-DCM (0.15 equiv.) in DME/2M Na₂CO₃ (3:1), at a concentration of 0.5 M, was stirred at 120° C. for 1.5 hours. The reaction was filtered and washed with EtOAc. The organic was partitioned with H₂O (25 mL), washed with NaCl_{(sat.)} (25 mL), dried over MgSO₄, and the volatiles were removed in vacuo. The residue was diluted in EtOAc and passed through a silica gel plug and the volatiles were removed in vacuo yielding methyl 3-amino-6-(2-fluoro-5-isopropylcabamoyl)phenyl)picolinate (60%). LCMS (m/z): 332.2 (MH⁺); LC Rₜ=2.9 min.

### Synthesis of 3-amino-6-(2-fluoro-5-isopropylcabamoyl)phenyl)picolinic acid

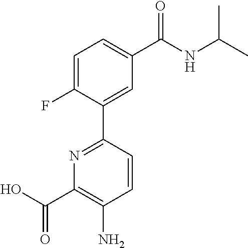 [SMILES: CC(C)NC(=O)c1ccc(F)c(-c2ccc(N)c(C(=O)O)n2)c1]

To a solution of methyl 3-amino-6-(2-fluoro-5-isopropylcabamoyl)phenyl)picolinate (1.0 equiv) in THF (0.5M), was added 1M LiOH (4.0 equiv). After stirring for 4 hours at 60° C., 1 N HCl (4.0 equiv.) was added and the THF was removed in vacuo. The resulting solid was filtered and rinsed with cold H₂O (3×20 mL) to yield 3-amino-6-(2-fluoro-5-isopropylcabamoyl)phenyl)picolinic acid (98%). LCMS (m/z): 318.1 (MH⁺); LC Rₜ=2.4 min.

### Synthesis of 2-(2,6-difluorophenyl)-3-fluoro-6-methylpyridine

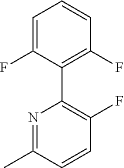 [SMILES: Cc1ccc(F)c(-c2c(F)cccc2F)n1]

To a solution of 2-bromo-3-fluoro-6-methylpyridine (1.0 equiv.) in THF and Water (10:1, 0.2 M) was added 2,6-difluorophenylboronic acid (2.0 equiv.) and potassium fluoride (3.3 equiv.). The reaction was degassed for 10 minutes, then Pd₂(dba)₃ (0.05 equiv.) was added, followed by tri-t-butylphosphine (0.1 equiv.). The reaction was stirred to 60° C. for 1 hour at which point, all starting material was consumed as indicated by LC/MS. The reaction was allowed to cool to room temperature, partitioned with ethyl acetate and water, the organic phase was dried with sodium sulfate, filtered, and concentrated. The crude material was diluted in EtOH to 0.1 M, and 0.5 equiv. of NaBH₄ was added to reduce the dba. The reaction was stirred for one hour at room temperature, then quenched with water and concentrated under vacuo to remove the ethanol. The product was extracted in ether, washed with brine, the organics were dried over sodium sulfate, filtered, and concentrated. The crude material was loaded on silica gel and purified via column chromatography (ISCO) eluting with hexanes and ethyl acetate (0%-10% ethyl acetate). The pure fractions were combined, and concentrated to yield 2-(2,6-difluorophenyl)-3-fluoro-6-methylpyridine as a light yellow oil in 86% yield. LC/MS=224.0 (M+H), Rₜ=0.84 min.

### Synthesis of 6-(2,6-difluorophenyl)-5-fluoropicolinic acid

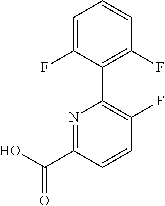 [SMILES: O=C(O)c1ccc(F)c(-c2c(F)cccc2F)n1]

To a solution of 2-(2,6-difluorophenyl)-3-fluoro-6-methylpyridine (1.0 equiv.) in water (0.05 M) was added KMnO₄ (2.0 equiv.) and the reaction was heated to reflux overnight. Another 2.0 equiv. of KMnO₄ were added and stirred at reflux for another 8 hours. The solution was cooled to room temperature, filtered through Celite and washed with water. The filtrate was acidified with 6N HCl to pH=3, the white precipitate was filtered. The filtrate was further acidified to pH=1 and filtered again. The filtrate was extracted with ethyl acetate until no more product in the aqueous layer. The organic phase was washed with brine and dried over magnesium sulfate, filtered, and concentrated. The residue was dissolved in ethyl acetate, washed with 1N NaOH, the aqueous layer was acidified to pH=1 and the white crystals were filtered. The combined products yielded 6-(2,6-difluorophenyl)-5-fluoropicolinic acid in 32% yield as a white solid. LC/MS=254.0 (M+H), Rₜ=0.71 min.

### Synthesis of methyl 3-amino-6-(thiazol-2-yl)picolinate

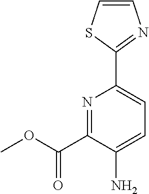 [SMILES: COC(=O)c1nc(-c2nccs2)ccc1N]

A solution of methyl 3-amino-6-bromopicolinate (1.0 equiv.), 2-thiazolylzinc bromide 0.5 M solution in THF (3.0 equiv.), and Pd(dppf)Cl₂-DCM (0.05 equiv.) was stirred at 80° C. for 1.5 hours. The reaction was filtered and washed with EtOAc. The organic was washed with H₂O (100 mL), and further washed with NaCl_{(sat.)} (50 mL), dried over MgSO₄, and the volatiles were removed in vacuo. The product was crystallized with hexane/EtOAc (1:1) to yield methyl 3-amino-6-(thiazol-2-yl)picolinate (51%). LCMS (m/z): 236.1 (MH⁺); LC Rₜ=2.3 min.

### Synthesis of 3-amino-6-(thiazol-2-yl)picolinic acid

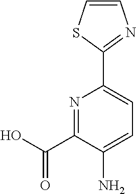 [SMILES: Nc1ccc(-c2nccs2)nc1C(=O)O]

To a solution of methyl 3-amino-6-(thiazol-2-yl)picolinate (1.0 equiv) in THF (0.5M), was added 1M LiOH (4.0 equiv). After stirring for 4 hours at 60° C., 1 N HCl (4.0 equiv.) was added and the THF was removed in vacuo. The resulting solid was filtered and rinsed with cold H₂O (3×20 mL) to yield 3-amino-6-(thiazol-2-yl)picolinic acid (61%). LCMS (m/z): 222.1 (MH⁺); LC Rₜ=1.9 min.

### Synthesis of 2,4-difluoro-N-isopropyl-3-(4,4,5,5-tetramethyl-1,3,2-dioxaborolan-2-yl)benzamide

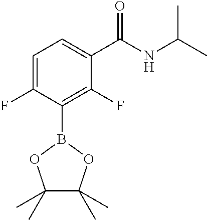 [SMILES: CC(C)NC(=O)c1ccc(F)c(B2OC(C)(C)C(C)(C)O2)c1F]

To a microwave vessel was added 3-bromo-2,4-difluoro-N-isopropylbenzamide (1 equiv.), 4,4,4′,4′,5,5,5′,5′-octamethyl-2,2′-bi(1,3,2-dioxaborolane) (1.5 equiv.), tricyclohexylphosphine (0.3 equiv.), Pd₂(dba)₃ (0.05 equiv.) and dioxane (0.3 M). After degassed for 15 min, potassium acetate (4 equiv.) was added. The reaction mixture was microwaved at 120° C. for 10 min. The crude product was diluted with EtOAc, which was filtered though Celite pad. The volatile material was removed to afford crude 2,4-difluoro-N-isopropyl-3-(4,4,5,5-tetramethyl-1,3,2-dioxaborolan-2-yl)benzamide, which was used for the next step without further purification. LCMS (m/z): 243.8 (MH+ of 2,6-difluoro-3-(isopropylcarbamoyl)phenylboronic acid), Rₜ=0.42 min.

### Synthesis of methyl 3-amino-6-(2,6-difluoro-3-(isopropylcarbamoyl)-phenyl)picolinate

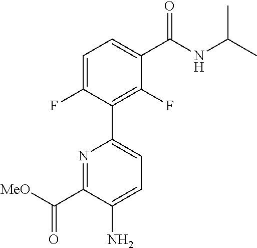 [SMILES: COC(=O)c1nc(-c2c(F)ccc(C(=O)NC(C)C)c2F)ccc1N]

To a microwave vessel, methyl 3-amino-6-bromopicolinate (700 mg, 1 equiv.), 2,4-difluoro-N-isopropyl-3-(4,4,5,5-tetramethyl-1,3,2-dioxaborolan-2-yl)benzamide (2 equiv.), PdCl₂(dppf) (0.1 equiv.), DME and 2 M Na₂CO₃ solution (3:1, 0.1 M solution) were added. The reaction mixture was degassed by N₂ stream for 10 min. After sealed, the reaction mixture was heated at 80° C. for 10 min in microwave. After 2 equiv. of bronic ester was added more, the reaction was repeated at microwave under the same condition. LCMS (m/z): 350.0 (MH⁺), Rₜ=0.67 min. ¹H-NMR (400 MHz, CDCl₃): δ 8.14 (m, 1H), 7.38 (m, 1H), 7.17 (m, 1H), 7.06 (m, 1H), 6.51 (m, 1H), 5.98 (s, 2H), 4.32 (m, 1H), 3.98 (s, 3H), 1.23 (s, 3H), 1.19 (s, 3H).

### Synthesis of 3-amino-6-(2,6-difluoro-3-(isopropylcarbamoyl)phenyl)-picolinic acid

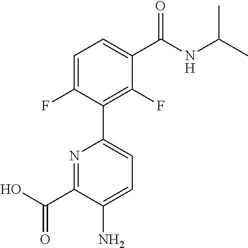 [SMILES: CC(C)NC(=O)c1ccc(F)c(-c2ccc(N)c(C(=O)O)n2)c1F]

To a solution of methyl 3-amino-6-(2,6-difluoro-3-(isopropylcarbamoyl)phenyl)picolinate (1 equiv.) in THF and MeOH (2:1, 0.2 M solution) was added aqueous LiOH solution (1 M) (1.5 equiv.). The reaction mixture was stirred for 1 h at room temperature. After the reaction mixture was neutralized with 1 N HCl solution (1.5 equiv.) and worked up with EtOAc, the crude 3-amino-6-(2,6-difluoro-3-(isopropylcarbamoyl)phenyl)picolinic acid was obtained in 65% yield. The crude product was used for the next step without further purification. LCMS (m/z): 336.9 (MH+), Rₜ=0.61 min.

### Method 3

### Synthesis of 2-(2,6-difluorophenyl)pyrimidine-4-carboxylic acid

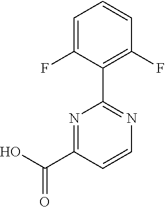 [SMILES: O=C(O)c1ccnc(-c2c(F)cccc2F)n1]

To a solution of 2-chloropyrimidine-4-carboxylic acid (1.0 equiv.) in DME and 2M Na₂CO₃ (3:1, 0.25 M) was added 2,6-difluorophenylboronic acid (1.3 equiv.) and Pd(dppf)Cl₂-DCM (0.05 equiv.) in a microwave vial. The vial was heated in the microwave at 120° C. for 30 minutes. The mixture was diluted with ethyl acetate and 1N NaOH was added. The organic phase was separated and extracted three more times with 1N NaOH and once with 6N NaOH. The combined aqueous phases were filtered and acidified to pH 1 by the addition of concentrated HCl and extracted with ethyl acetate. The organic layer was dried over magnesium sulfate, filtered, and concentrated to give 2-(2,6-difluorophenyl)pyrimidine-4-carboxylic acid in 81%. LCMS (m/z): 237.0 (MH⁺), Rₜ=0.54 min.

### Synthesis of 5-fluoro-6-(2-fluorophenyl)picolinic acid

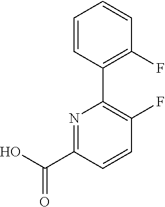 [SMILES: O=C(O)c1ccc(F)c(-c2ccccc2F)n1]

Method 3 was followed using 6-bromo-5-fluoropicolinic acid (1.0 equiv.) and 2-fluorophenylboronic acid (1.3 equiv.) and Pd(dppf)Cl₂-DCM (0.05 equiv.) to give 5-fluoro-6-(2-fluorophenyl)picolinic acid in 43% yield. LCMS (m/z): 236.1 (MH⁺), Rₜ=0.72 min.

### Synthesis of 6-(2-fluorophenyl)picolinic acid

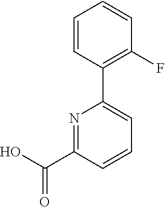 [SMILES: O=C(O)c1cccc(-c2ccccc2F)n1]

Method 3 was followed using 6-bromopicolinic acid (1.0 equiv.) and 2-fluorophenylboronic acid (1.5 equiv.) and Pd(dppf)Cl₂-DCM (0.05 equiv.) to give 6-(2-fluorophenyl)picolinic acid in 93% yield. LCMS (m/z): 218.0 (MH⁺), Rₜ=0.66 min.

### Synthesis of 6-(2,6-difluorophenyl)picolinic acid

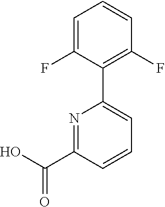 [SMILES: O=C(O)c1cccc(-c2c(F)cccc2F)n1]

Method 3 was followed using 6-bromopicolinic acid (1.0 equiv.) and 2,6-difluorophenylboronic acid (1.5 equiv.) and Pd(dppf)Cl₂-DCM (0.05 equiv.) to give 6-(2,6-difluorophenyl)picolinic acid in 38% yield. LCMS (m/z): 236.0 (MH⁺), Rₜ=0.87 min.

### Synthesis of 5-fluoro-6-(2-fluoro-5-(isopropylcarbamoyl)phenyl)picolinic acid

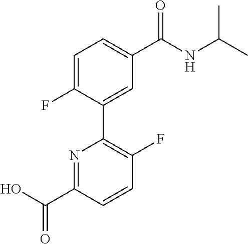 [SMILES: CC(C)NC(=O)c1ccc(F)c(-c2nc(C(=O)O)ccc2F)c1]

Method 3 was followed using 6-bromo-5-fluoropicolinic acid (1.0 equiv.) and 2-fluoro-5-(isopropylcarbamoyl)phenylboronic acid (1.5 equiv.) and Pd(dppf)Cl₂-DCM (0.05 equiv.) to give 5-fluoro-6-(2-fluoro-5-(isopropylcarbamoyl)phenyl)picolinic acid in 75% yield. LCMS (m/z): 320.9 (MH⁺), Rₜ=0.67 min.

### Method 4

### Synthesis of 5-amino-2-(2,6-difluorophenyl)pyrimidine-4-carboxylic acid

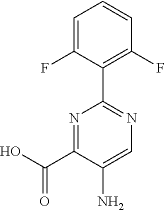 [SMILES: Nc1cnc(-c2c(F)cccc2F)nc1C(=O)O]

A 2.68 M NaOEt in EtOH solution (3 eq) was added to an ice-bath cooled mixture of 2,6-difluorobenzimidamide hydrochloride (2 eq) in EtOH (0.1 M). The resulting mixture was allowed to warm to rt and stirred under N₂ for 30 min. To the reaction mixture was added drop wise a solution of mucobromic acid (1 eq) in EtOH and the reaction was heated in a 50° C. oil bath for 2.5 hr. After cooling to rt the reaction mixture was concentrated in vacuo. H₂0 and 1.0 N NaOH were added and the aqueous mixture was washed with EtOAc. The aqueous phase was acidified to pH=4 with 1.0 N HCl then extracted with EtOAc. Combined organic extracts were washed once with brine, then dried over anhydrous Na₂SO₄, filtered, and concentrated in vacuo to give 5-bromo-2-(2,6-difluorophenyl)pyrimidine-4-carboxylic acid. The crude product was used for the next step without further purification. LC/MS (m/z): 316.9 (MH⁺). LC: Rₜ: 2.426 min.

CuSO₄ (0.1 eq) was added to a mixture of 5-bromo-2-(2,6-difluorophenyl)pyrimidine-4-carboxylic acid (1 eq) and 28% aqueous ammonium hydroxide solution in a microwave reaction vessel. The reaction mixture was heated in a microwave reactor at 110° C. for 25 min. The reaction vessel was cooled in dry ice for 30 min then unsealed and concentrated in vacuo. To the resulting solids was added 1.0 N HCl and the mixture was extracted with EtOAc. Combined organic extracts were washed once with brine, then dried over anhydrous Na₂SO₄, filtered, and concentrated in vacuo to give 5-amino-2-(2,6-difluorophenyl)pyrimidine-4-carboxylic acid. The crude product was used for the next step without further purification. LCMS (m/z): 252.0 (MH⁺), Rₜ=2.0 min.

### Synthesis of 5-amino-2-(2-fluorophenyl)pyrimidine-4-carboxylic acid

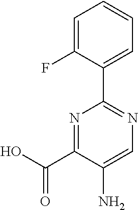 [SMILES: Nc1cnc(-c2ccccc2F)nc1C(=O)O]

Following METHOD 4,5-amino-2-(2-fluorophenyl)pyrimidine-4-carboxylic acid was prepared starting from 2-fluorobenzimidamide hydrochloride. LC/MS (m/z): 234.0 (MH⁺), Rₜ: 0.70 min.

### Synthesis of 5-amino-2-phenylpyrimidine-4-carboxylic acid

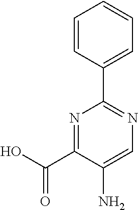 [SMILES: Nc1cnc(-c2ccccc2)nc1C(=O)O]

Following METHOD 4,5-amino-2-phenylpyrimidine-4-carboxylic acid was prepared starting from benzimidamide hydrochloride. LC/MS (m/z): 216.1 (MH⁺).

### Synthesis of 6-(2,6-difluoro-3-nitrophenyl)-5-fluoropicolinic acid

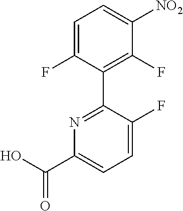 [SMILES: O=C(O)c1ccc(F)c(-c2c(F)ccc([N+](=O)[O-])c2F)n1]

To a solution of 6-(2,6-difluorophenyl)-5-fluoropicolinic acid (1.0 equiv.) in H₂SO₄ (5.0 equiv.) was added fuming nitric acid (6.0 equiv.) mixture slowly at room temperature. The reaction mixture was stirred at room temperature for 2 h. The reaction mixture was poured into ice resulting in the formation of a white precipitate. The precipitate was collected by filteration and dried in air for 10 min followed by in vaccuo overnight to yield 6-(2,6-difluoro-3-nitrophenyl)-5-fluoropicolinic acid in 85% yield. LC/MS=298.9 (M+H), Rₜ=0.67 min. ¹H NMR (400 MHz, <dmso>) δ ppm 7.45-7.68 (m, 1H), 8.04-8.20 (m, 1H), 8.24-8.36 (m, 1H), 8.46 (td, J=9.00, 5.48 Hz, 1H).

### Synthesis of methyl 6-(2,6-difluoro-3-nitrophenyl)-5-fluoropicolinate

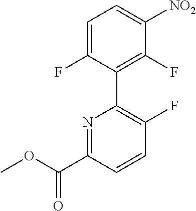 [SMILES: COC(=O)c1ccc(F)c(-c2c(F)ccc([N+](=O)[O-])c2F)n1]

To a solution of 6-(2,6-difluoro-3-nitrophenyl)-5-fluoropicolinic acid (1.0 equiv.) in MeOH (0.11 M) at RT was added sulfuric acid (4.2 equiv.) dropwise. The resulting solution was stirred at RT for 18 h. The reaction mixture was diluted with EtOAc and quenched slowly with NaHCO₃. The aqeuous layer was then separated and extracted with EtOAc, the combined organic layers were then dried over MgSO₄ and concentrated in vacuo to yield methyl 6-(2,6-difluoro-3-nitrophenyl)-5-fluoropicolinate in 99% yield. ¹H NMR (400 MHz, <cdcl3>) δ ppm 4.02 (s, 3H), 7.10-7.24 (m, 1H), 7.68-7.80 (m, 1H), 8.18-8.32 (m, 1H), 8.32-8.40 (m, 1H).

### Synthesis of methyl 6-(3-amino-2,6-difluorophenyl)-5-fluoropicolinate

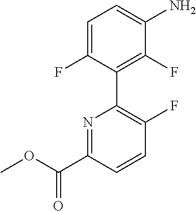 [SMILES: COC(=O)c1ccc(F)c(-c2c(F)ccc(N)c2F)n1]

A suspension of methyl 6-(2,6-difluoro-3-nitrophenyl)-5-fluoropicolinate (1.0 equiv.) and iron powder (6.0 equiv.) in acetic acid (8.5 M) was rapidly stirred at RT for 16 h. The reaction mixture was diluted with EtOAc, then quenched with sat. aq. Na₂CO₃. The aqueous layer was then separated and extracted with EtOAc. The combined organics were then dried over MgSO₄ and concentrated in vaccuo. The foam was further purified by column chromatography eluting with 100% heptane to 30% EtOAc:heptane to 50% EtOAc:heptane to yield methyl 6-(3-amino-2,6-difluorophenyl)-5-fluoropicolinate in 68% yield. LC/MS=283.0 (M+H), Rt=0.61 min. ¹H NMR (400 MHz, <cdcl3>) δ ppm 3.92-4.09 (m, 3H), 6.71-6.93 (m, 2H), 7.56-7.72 (m, 1H), 8.17-8.34 (m, 1H).

### Synthesis of methyl 6-(3-acetamido-2,6-difluorophenyl)-5-fluoropicolinate

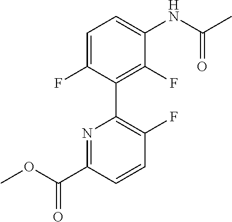 [SMILES: COC(=O)c1ccc(F)c(-c2c(F)ccc(NC(C)=O)c2F)n1]

To a solution of methyl 6-(3-amino-2,6-difluorophenyl)-5-fluoropicolinate (1.0 equiv.) and N-ethyl-N-isopropylpropan-2-amine (3.0 equiv.) in THF (0.10 M) at rt was added acetyl chloride (2.0 equiv.). The mixture was stirred at rt for 5 hrs. The reaction mixture was diluted with EtOAc then quenched with sat. aq. Na₂CO₃. The aqueous layer was then separated and extracted with EtOAc. The combined organics were then dried over MgSO₄ and concentrated in vacuo The foam was further purified by column chromatography eluting with 100% heptane to 30% EtOAc:heptane to 50% EtOAc:heptane to yield methyl 6-(3-acetamido-2,6-difluorophenyl)-5-fluoropicolinate in 78% yield. LC/MS=324.9 (M+H), Rt=0.64 min. ¹H NMR (400 MHz, <cdcl3>) δ ppm 2.14-2.31 (m, 3H), 3.93-4.08 (m, 3H), 6.90-7.08 (m, 1H), 7.30-7.45 (m, 1H), 7.60-7.73 (m, 1H), 8.20-8.32 (m, 1H), 8.34-8.49 (m, 1H).

### Synthesis of 6-(3-acetamido-2,6-difluorophenyl)-5-fluoropicolinic acid

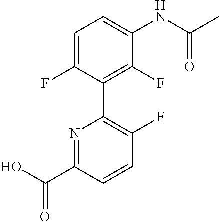 [SMILES: CC(=O)Nc1ccc(F)c(-c2nc(C(=O)O)ccc2F)c1F]

Method 2 was followed using methyl 6-(3-acetamido-2,6-difluorophenyl)-5-fluoropicolinate (1.0 equiv.) and LiOH (5.5 equiv.) to give 6-(3-acetamido-2,6-difluorophenyl)-5-fluoropicolinic acid in 93% yield. LC/MS=310.9 (M+H), Rₜ=0.56 min. ¹H NMR (400 MHz, <dmso>) δ ppm 1.97-2.11 (m, 3H), 7.22 (t, J=8.61 Hz, 1H), 7.83-7.98 (m, 1H), 8.00-8.09 (m, 1H), 8.14-8.25 (m, 1H), 9.82 (s, 1H).

### Synthesis of methyl 6-(2,6-difluoro-3-isobutyramidophenyl)-5-fluoropicolinate

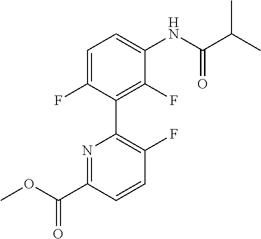 [SMILES: COC(=O)c1ccc(F)c(-c2c(F)ccc(NC(=O)C(C)C)c2F)n1]

To a solution of methyl 6-(3-amino-2,6-difluorophenyl)-5-fluoropicolinate (1.0 equiv.) and N-ethyl-N-isopropylpropan-2-amine (3.0 equiv.) in THF (0.10 M) at rt was added isobutyryl chloride (2.0 equiv.). The mixture was stirred at rt for 5 hrs. The reaction mixture was diluted with EtOAc then quenched with sat. aq. Na₂CO₃. The aqueous layer was then separated and extracted with EtOAc. The combined organics were then dried over MgSO₄ and concentrated in vaccuo The foam was further purified by column chromatography eluting with 100% heptane to 30% EtOAc:heptane to 50% EtOAc:heptane to yield methyl 6-(2,6-difluoro-3-isobutyramidophenyl)-5-fluoropicolinate in 88% yield. LC/MS=352.9 (M+H), Rt=0.76 min.

### Synthesis of 6-(2,6-difluoro-3-isobutyramidophenyl)-5-fluoropicolinic acid

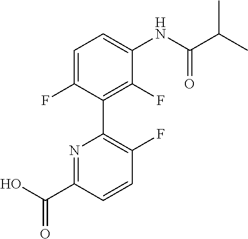 [SMILES: CC(C)C(=O)Nc1ccc(F)c(-c2nc(C(=O)O)ccc2F)c1F]

Method 2 was followed using methyl 6-(2,6-difluoro-3-isobutyramidophenyl)-5-fluoropicolinate (1.0 equiv.) and LiOH (5.5 equiv.) to give 6-(2,6-difluoro-3-isobutyramidophenyl)-5-fluoropicolinic acid in 98% yield. LC/MS=338.9 (M+H), Rₜ=0.66 min. ¹H NMR (400 MHz, <dmso>) δ ppm 1.01-1.09 (m, 6H), 2.57-2.73 (m, 1H), 7.22 (t, J=9.00 Hz, 1H), 7.87 (td, J=8.80, 6.26 Hz, 1H), 7.95-8.11 (m, 1H), 8.13-8.27 (m, 1H), 9.55-9.77 (m, 1H).

### Method 5

### Synthesis of methyl 6-(2,6-difluoro-4-methoxyphenyl)-5-fluoropicolinate

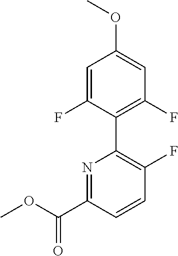 [SMILES: COC(=O)c1ccc(F)c(-c2c(F)cc(OC)cc2F)n1]

To a degassed suspension of methyl 6-bromo-5-fluoropicolinate (1.0 equiv.), 2,6-difluoro-4-methoxyphenylboronic acid (2.5 equiv.) and potassium fluoride (3.3 equiv.) in THF/Water (10/1, 0.19 M) was added Pd₂(dba)₃ (0.2 equiv.) and P(tBu)₃ in toluene (0.4 equiv.). The reaction mixture was sealed and heated under microwave irradiation at 100° C. for 15 min. The reaction mixture was quenched with water and diluted with EtOAc. The aqueous layer was separated and reextracted with EtOAc. The combined organics were then dried over MgSO₄ and concentrated in vaccuo. The crude was further purified by column chromatography eluting with 100% heptane to 10% EtOAc:heptane to 75% EtOAc:heptane to yield methyl 6-(2,6-difluoro-4-methoxyphenyl)-5-fluoropicolinate in 85% yield. LC/MS=298.0 (M+H), Rt=0.89 min.

### Synthesis of 6-(2,6-difluoro-4-methoxyphenyl)-5-fluoropicolinic acid

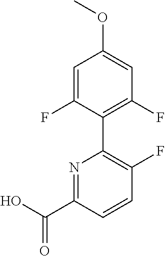 [SMILES: COc1cc(F)c(-c2nc(C(=O)O)ccc2F)c(F)c1]

To a solution of methyl 6-(2,6-difluoro-4-methoxyphenyl)-5-fluoropicolinate (1.0 equiv.) in THF/MeOH (2:1, 0.09 M) was added LiOH (1.5 equiv.) and the reaction was stirred at room temperature for 1 hour. The solution was quenched with 1N HCl, extracted with ethyl acetate, washed with brine, dried with sodium sulfate, filtered and concentrated to give 6-(2,6-difluoro-4-methoxyphenyl)-5-fluoropicolinic acid in 84% yield. LC/MS=284.1 (M+H), Rt=0.76 min.

### Synthesis of 2-(2,6-difluoro-4-methylphenyl)-4,4,5,5-tetramethyl-1,3,2-dioxaboroane

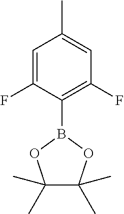 [SMILES: Cc1cc(F)c(B2OC(C)(C)C(C)(C)O2)c(F)c1]

To a solution of 1,3-difluoro-5-methylbenzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO₃ (sat.) and extracted with EtOAc. The organics were washed with brine, dried over Na₂SO₄, filtered and concentrated to yield 2-(2,6-difluoro-4-methylphenyl)-4,4,5,5-tetramethyl-1,3,2-dioxaboroane as a white solid in 92%. ¹H NMR (400 MHz, <cdcl3>) δ ppm 6.67 (dd, J=9.39, 0.78 Hz, 2H), 2.34 (s, 3H), 1.38 (s, 12H).

### Synthesis of 6-(2,6-difluoro-4-methylphenyl)-5-fluoropicolinate

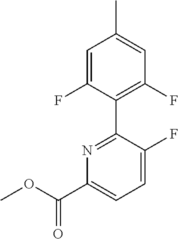 [SMILES: COC(=O)c1ccc(F)c(-c2c(F)cc(C)cc2F)n1]

Method 5 was followed using 6-bromo-5-fluoropicolinate (1.0 equiv.) and 2-(2,6-difluoro-4-methylphenyl)-4,4,5,5-tetramethyl-1,3,2-dioxaboroane (1.75 equiv.) to give methyl 6-(2,6-difluoro-4-methylphenyl)-5-fluoropicolinate as a solid in 85% yield. LC/MS=282.0 (M+H), Rt=0.87 min.

### Synthesis of 6-(2,6-difluoro-4-methylphenyl)-5-fluoropicolinic acid

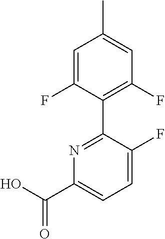 [SMILES: Cc1cc(F)c(-c2nc(C(=O)O)ccc2F)c(F)c1]

To a solution of 6-(2,6-difluoro-4-methylphenyl)-5-fluoropicolinate (1.0 eq) in THF (0.1M) was added LiOH (5.5 eq, 2M) and allowed to stir at room temperature for 4 hrs. The volatiles were removed in vacuo, and the residual aqueous was acidified with 2M HCl to pH 4. The precipitate was filtered and dried to yield 6-(2,6-difluoro-4-methylphenyl)-5-fluoropicolinic acid as al light yellow solid in 73.5%. LCMS (m/z): 268.0 (MH⁺), Rₜ=0.76 min.

### Synthesis of 2-(2,6-difluoro-4-(methylthio)phenyl)-4,4,5,5-tetramethyl-1,3,2-dioxaborolane

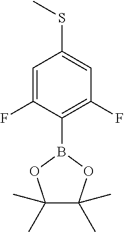 [SMILES: CSc1cc(F)c(B2OC(C)(C)C(C)(C)O2)c(F)c1]

To a solution of (3,5-difluorophenyl)(methyl)sulfane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield a 2-(2,6-difluoro-4-(methylthio)phenyl)-4,4,5,5-tetramethyl-1,3,2-dioxaborolane in 91%. ¹H NMR (400 MHz, <cdcl3>) δ ppm 6.71 (dd, 2H), 2.48 (s, 3H), 1.37 (s, 12H).

### Synthesis of methyl 6-(2,6-difluoro-4-(methylthio)phenyl)-5-fluoropicolinate

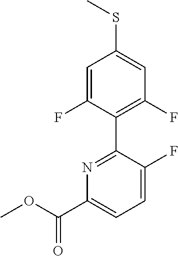 [SMILES: COC(=O)c1ccc(F)c(-c2c(F)cc(SC)cc2F)n1]

Method 5 was followed using 6-bromo-5-fluoropicolinate (1.0 equiv.) and 2-(2,6-difluoro-4-(methylthio)phenyl)-4,4,5,5-tetramethyl-1,3,2-dioxaborolane (1.75 equiv.) to give methyl 6-(2,6-difluoro-4-(methylthio)phenyl)-5-fluoropicolinate in 73% yield. LC/MS=313.9 (M+H), Rt=0.90 min.

### Synthesis of methyl 6-(2,6-difluoro-4-(methylsulfonyl)phenyl)-5-fluoropicolinate

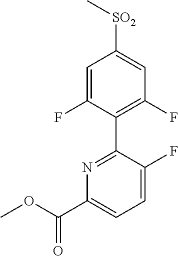 [SMILES: COC(=O)c1ccc(F)c(-c2c(F)cc(S(C)(=O)=O)cc2F)n1]

To a solution of methyl 6-(2,6-difluoro-4-(methylthio)phenyl)-5-fluoropicolinate (1.0 equiv) in CH₂Cl₂ (0.2 M) at 0° C. was added MCPBA (3.2 equiv.). After stirring for 40 minutes, the reaction was quenched with Na₂S₂O_{3(aq.)}, diluted with EtOAc, washed with NaHCO_{3(sat.)}, brine, dried over MgSO₄, filtered, concentrate, purified by SiO₂ chromatography to yield methyl 6-(2,6-difluoro-4-(methylsulfonyl)phenyl)-5-fluoropicolinate in 56% yield. LC/MS=345.9 (M+H), Rt=0.69 min.

### Synthesis of 6-(2,6-difluoro-4-(methylsulfonyl)phenyl)-5-fluoropicolinic acid

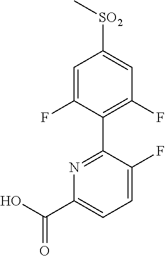 [SMILES: CS(=O)(=O)c1cc(F)c(-c2nc(C(=O)O)ccc2F)c(F)c1]

To a solution of 6-(2,6-difluoro-4-(methylsulfonyl)phenyl)-5-fluoropicolinate (1.0 eq) in THF (0.1M) was added LiOH (5.5 eq, 2M) and allowed to stir at 37° C. for 2 hrs. The volatiles were removed in vacuo, and the residual aqueous was acidified with 2M HCl to pH 4. The precipitate was filtered and dried to yield 6-(2,6-difluoro-4-(methylsulfonyl)phenyl)-5-fluoropicolinic acid as a solid in 91% yield. LCMS (m/z): 331.8 (MH⁺), Rₜ=0.59 min.

### Synthesis of tert-butyl(3,5-difluorophenoxy)dimethylsilane

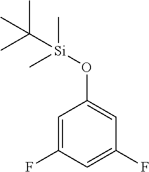 [SMILES: CC(C)(C)[Si](C)(C)Oc1cc(F)cc(F)c1]

To a solution of 3,5-difluorophenol (1.0 equiv.) and imidazole (2.2 equiv.) in DMF (0.8 M) at 0° C. was added TBDMSCl (1.1 equiv.). The ice bath was removed and after stirring for 3 hours the solution was diluted with EtOAc, washed with water, brine, dried over MgSO₄, filtered, concentrated and purified by SiO₂ chromatography to yield tert-butyl(3,5-difluorophenoxy)dimethylsilane in 73% yield. ¹H NMR (400 MHz, <cdcl3>) δ ppm 0.23 (s, 6H) 0.99 (s, 9H) 6.33-6.40 (m, 2H) 6.44 (tt 1H).

### Synthesis of tert-butyl(3,5-difluoro-4-(4,4,5,5-tetramethyl-1,3,2-dioxaborolan-2-yl)phenoxy)dimethylsilane

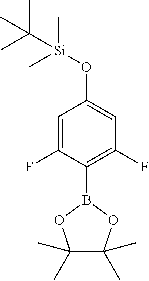 [SMILES: CC1(C)OB(c2c(F)cc(O[Si](C)(C)C(C)(C)C)cc2F)OC1(C)C]

To a solution of tert-butyl(3,5-difluorophenoxy)dimethylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield tert-butyl(3,5-difluoro-4-(4,4,5,5-tetramethyl-1,3,2-dioxaborolan-2-yl)phenoxy)dimethylsilane in 91% yield. ¹H NMR (400 MHz, <cdcl3>) δ ppm 0.21 (s, 6H) 0.97 (s, 9H) 1.37 (s, 12H) 6.33 (d, J=9.39 Hz, 2H).

### Synthesis of methyl 6-(2,6-difluoro-4-hydroxyphenyl)-5-fluoropicolinate

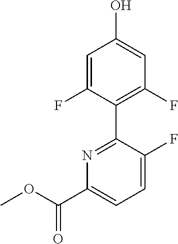 [SMILES: COC(=O)c1ccc(F)c(-c2c(F)cc(O)cc2F)n1]

Method 5 was followed using 6-bromo-5-fluoropicolinate (1.0 equiv.) and tert-butyl(3,5-difluoro-4-(4,4,5,5-tetramethyl-1,3,2-dioxaborolan-2-yl)phenoxy)dimethylsilane (1.75 equiv.) to give methyl 6-(2,6-difluoro-4-hydroxyphenyl)-5-fluoropicolinate in 65% yield. The reaction was heated for an additional 30 minutes at 100° C. in the microwave to drive to completion the deprotection of the TBDMS group. LC/MS=283.9 (M+H), Rt=0.69 min.

### Synthesis of methyl 6-(4-(2-(tert-butyldimethylsilyloxy)ethoxy)-2,6-difluorophenyl)-5-fluoropicolinate

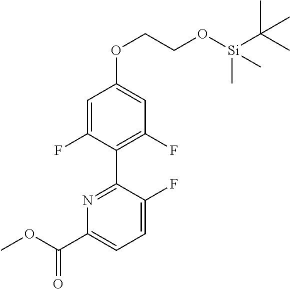 [SMILES: COC(=O)c1ccc(F)c(-c2c(F)cc(OCCO[Si](C)(C)C(C)(C)C)cc2F)n1]

To a solution of methyl 6-(2,6-difluoro-4-hydroxyphenyl)-5-fluoropicolinate (1.0 equiv.) and potassium carbonate (4.0 equiv.) in DMF (0.4 M) was added (2-bromoethoxy)(tert-butyl)dimethylsilane (2 equiv.). After stirring for 72 hours at rt the heterogeneous solution was diluted with water, extracted with EtOAc, dried over MgSO₄, filtered, concentrated and purified by ISCO SiO₂ chromatography to yield methyl 6-(4-(2-(tert-butyldimethylsilyloxy)ethoxy)-2,6-difluorophenyl)-5-fluoropicolinate in 74% yield. LC/MS=442.1 (M+H), Rₜ=1.22 min.

### Synthesis of 6-(4-(2-(tert-butyldimethylsilyloxy)ethoxy)-2,6-difluorophenyl)-5-fluoropicolinic acid

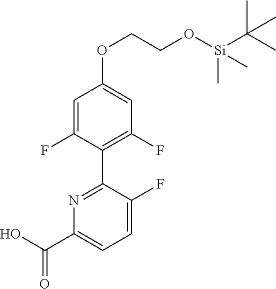 [SMILES: CC(C)(C)[Si](C)(C)OCCOc1cc(F)c(-c2nc(C(=O)O)ccc2F)c(F)c1]

Method 2 was followed using methyl 6-(4-(2-(tert-butyldimethylsilyloxy)ethoxy)-2,6-difluorophenyl)-5-fluoropicolinate to give 6-(4-(2-(tert-butyldimethylsilyloxy)ethoxy)-2,6-difluorophenyl)-5-fluoropicolinic acid in 94% yield. LC/MS=428.1 (M+H), Rₜ=1.13 min.

### Synthesis of 1,3-difluoro-5-(2-methoxyethoxy)benzene

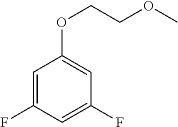

To a solution of 3,5-difluorophenol (1.0 equiv.), 2-methoxyethanol (3.0 equiv.) and triphenylphosphine (3.0 equiv) in THF (0.1 M) was added DIAD (3.0 equiv.). After stirring at rt for 18 hours, the volatiles were removed in vacuo and the residue was purified by SiO₂ chromatography to yield 1,3-difluoro-5-(2-methoxyethoxy)benzene in 95% yield. ¹H NMR (400 MHz, <cdcl3>) δ ppm 6.41-6.47 (m, 3H), 4.08 (t, 2H), 3.74 (t, 2H), 3.45 (s, 3H).

### Synthesis of 2-(2,6-difluoro-4-(2-methoxyethoxy)phenyl)-4,4,5,5-tetramethyl-1,3,2-dioxaborolane

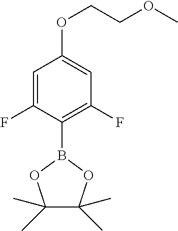 [SMILES: COCCOc1cc(F)c(B2OC(C)(C)C(C)(C)O2)c(F)c1]

To a solution of 1,3-difluoro-5-(2-methoxyethoxy)benzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield 2-(2,6-difluoro-4-(2-methoxyethoxy)phenyl)-4,4,5,5-tetramethyl-1,3,2-dioxaborolane. ¹H NMR (400 MHz, <cdcl3>) δ ppm 6.42 (d, 2H), 4.10 (m, 2H), 3.74 (m, 2H), 3.44 (s, 3H), 1.37 (s, 12H).

### Synthesis of methyl 6-(2,6-difluoro-4-(2-methoxyethoxy)phenyl)-5-fluoropicolinate

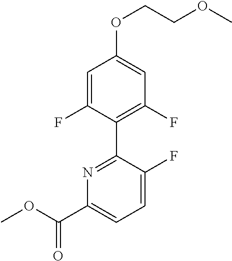

Method 5 was followed using methyl 6-bromo-5-fluoropicolinate (1.0 equiv.) and 2-(2,6-difluoro-4-(2-methoxyethoxy)phenyl)-4,4,5,5-tetramethyl-1,3,2-dioxaborolane (1.75 equiv.) at 80° C. for 1 hour in the oil bath to give methyl 6-(2,6-difluoro-4-(2-methoxyethoxy)phenyl)-5-fluoropicolinate in 95% yield. LC/MS=341.9 (M+H), Rₜ=0.89 min.

### Synthesis of 6-(2,6-difluoro-4-(2-methoxyethoxy)phenyl)-5-fluoropicolinic acid

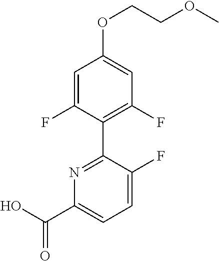 [SMILES: COCCOc1cc(F)c(-c2nc(C(=O)O)ccc2F)c(F)c1]

Method 2 was followed using methyl 6-(2,6-difluoro-4-(2-methoxyethoxy)phenyl)-5-fluoropicolinate to give 6-(2,6-difluoro-4-(2-methoxyethoxy)phenyl)-5-fluoropicolinic acid in 98% yield. LC/MS=327.9 (M+H), Rₜ=0.71 min.

### Synthesis of (2-(3,5-difluorophenyl)propan-2-yloxy)triisopropylsilane

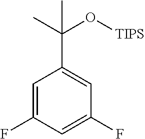

To a solution of 1-(3,5-difluorophenyl)ethanone (1.0 equiv) in THF (0.2 M) at 0° C. was added methylmagnesium bromide (1.0 M in THF, 1.15 equiv). After stirring for 4 hours the reaction was quenched by addition of NH₄Cl_{(sat.)}, diluted with EtOAc, washed with NaCl_{(sat.)}, dried over MgSO₄, filtered, concentrated and purified by ISCO SiO₂ chromatography to yield 2-(3,5-difluorophenyl)propan-2-ol. To a solution of 2-(3,5-difluorophenyl)propan-2-ol in CH₂Cl₂ (0.1 M) at 0° C. was added 2,6 lutidine (6 equiv.) and than triisopropylsilyl trifluoromethanesulfonate (3.0 equiv.). After stirring for 3 hours at 0° C. and six hours at rt the solution was partitioned between EtOAc and NaHCO_{3(sat.)}, separated, washed with NaCl_{(sat.)}, dried over MgSO₄, filtered, concentrated and purified by ISCO SiO₂ chromatography to yield (2-(3,5-difluorophenyl)propan-2-yloxy)triisopropylsilane. (400 MHz, <cdcl3>) δ ppm 1.05-1.08 (m, 21H) 1.57 (s, 6H) 6.63 (s, 1H) 7.00 (dd, J=9.39, 2.35 Hz, 2H).

### Synthesis of (2-(3,5-difluoro-4-(4,4,5,5-tetramethyl-1,3,2-dioxaborolan-2-yl)phenyl)propan-2-yloxy)triisopropylsilane

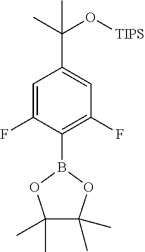

To a solution of (2-(3,5-difluorophenyl)propan-2-yloxy)triisopropylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield (2-(3,5-difluoro-4-(4,4,5,5-tetramethyl-1,3,2-dioxaborolan-2-yl)phenyl)propan-2-yloxy)triisopropylsilane in 99%. ¹H NMR (400 MHz, <cdcl3>) δ ppm 1.03-1.08 (m, 21H) 1.24 (s, 12H) 1.38 (s, 3H) 1.57 (s, 3H) 6.92-7.03 (m, 2H).

### Synthesis of methyl 6-(2,6-difluoro-4-(2-hydroxypropan-2-yl)phenyl)-5-fluoropicolinate

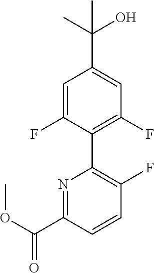 [SMILES: COC(=O)c1ccc(F)c(-c2c(F)cc(C(C)(C)O)cc2F)n1]

Method 5 was followed using 6-bromo-5-fluoropicolinate (1.0 equiv.) and (2-(3,5-difluoro-4-(4,4,5,5-tetramethyl-1,3,2-dioxaborolan-2-yl)phenyl)propan-2-yloxy)triisopropylsilane (1.6 equiv.) at 100° C. for 30 min in the microwave to give methyl 6-(2,6-difluoro-4-(2-hydroxypropan-2-yl)phenyl)-5-fluoropicolinate in 90% yield. LC/MS=325.9 (M+H), Rt=0.81 min. ¹H NMR (400 MHz, <cdcl3>) δ ppm 1.59 (s, 6H), 4.00 (s, 3H), 7.15 (d, J=9.00 Hz, 2H), 7.62-7.68 (m, 1H), 8.23-8.29 (m, 1H).

### Synthesis of 6-(2,6-difluoro-4-(2-hydroxypropan-2-yl)phenyl)-5-fluoropicolinic acid

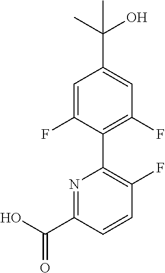 [SMILES: CC(C)(O)c1cc(F)c(-c2nc(C(=O)O)ccc2F)c(F)c1]

Method 2 was followed using methyl 6-(2,6-difluoro-4-(2-hydroxypropan-2-yl)phenyl)-5-fluoropicolinate to give 6-(2,6-difluoro-4-(2-hydroxypropan-2-yl)phenyl)-5-fluoropicolinic acid in 94% yield. LC/MS=312.0 (M+H), Rₜ=0.69 min.

### Synthesis of 2-(2-(2-fluorophenyl)hydrazono)acetaldehyde

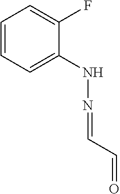 [SMILES: O=CC=NNc1ccccc1F]

A solution of (2-fluorophenyl)hydrazine (1.0 equov.) in water/AcOH (1/1, 0.77 M) were added slowly to a 40% aqueous solution of glyoxal (5.0 equiv.) over 30 min. The mixture was stirred at rt overnight. The mixture was filtered with a coarse frit glass funnel. The cake was washed with water and air dried for 1 h to yield 2-(2-(2-fluorophenyl)hydrazono)acetaldehyde in 97% yield. LC/MS (m/z): 166.9 (MH⁺), Rₜ=072 min. ¹H NMR (CDCl₃) δ: 9.63 (d, J=7.4 Hz, 1H), 8.97 (br. s., 1H), 7.64 (t, J=8.0 Hz, 1H), 7.31-7.37 (m, 1H), 7.05-7.20 (m, 2H), 6.93-7.03 (m, 1H).

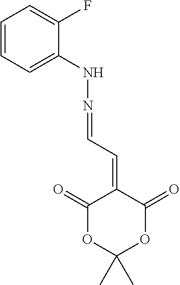 [SMILES: CC1(C)OC(=O)C(=CC=NNc2ccccc2F)C(=O)O1]

2-(2-(2-fluorophenyl)hydrazono)acetaldehyde (1.0 equiv.) was mixed with 2,2-dimethyl-1,3-dioxane-4,6-dione (1.0 equiv.) in toluene (0.33 M). 15 drops of acetic acid was added followed by 15 drops of diallylamine. The mixture was stirred overnight at rt. The solid was collected in a frit glass funnel, washed with Pentane and air dried to yield 5-(2-(2-(2-fluorophenyl)hydrazono)ethylidene)-2,2-dimethyl-1,3-dioxane-4,6-dione in 67% yield. ¹H NMR (400 MHz, CDCl₃) δ: 10.09 (br. s., 1H), 9.56 (br. s., 1H), 8.86 (t, J=10.6 Hz, 1H), 8.21-8.32 (m, 1H), 6.97-7.22 (m, 2H), 1.75 (d, J=5.1 Hz, 6H).

### Synthesis of 2-(2-fluorophenyl)-3-oxo-2,3-dihydropyridazine-4-carboxylic acid

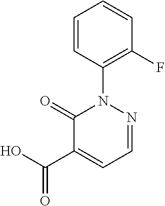 [SMILES: O=C(O)c1ccnn(-c2ccccc2F)c1=O]

5-(2-(2-(2-fluorophenyl)hydrazono)ethylidene)-2,2-dimethyl-1,3-dioxane-4,6-dione (1.0 equiv.) was dissolved in MeOH (0.20 M) and sodium methoxide (1.2 equiv.) was added. The mixture was heated at reflux for 17 h. Cold 1 N HCl was added and the mixture was extracted with DCM. The organics were washed with brine, dried over sodium sulfate, filtered, concentrated and co evaporated with diethylether to give 2-(2-fluorophenyl)-3-oxo-2,3-dihydropyridazine-4-carboxylic acid in 67% yield. LC/MS (m/z): 234.9 (MH+), Rₜ=0.59 min. ¹H NMR (DMSO) δ: 13.63 (br. s., 1H), 8.24 (d, J=3.9 Hz, 1H), 7.96 (d, J=3.9 Hz, 1H), 7.51-7.64 (m, 2H), 7.34-7.49 (m, 2H).

### Synthesis of methyl 6-(2,6-difluoro-3-(isopropylcarbamoyl)phenyl)-5-fluoropicolinate

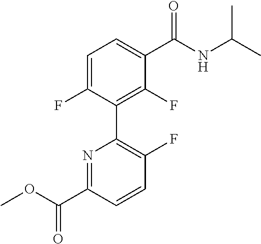 [SMILES: COC(=O)c1ccc(F)c(-c2c(F)ccc(C(=O)NC(C)C)c2F)n1]

Method 5 was followed using 6-bromo-5-fluoropicolinate (1.0 equiv.) and 2,4-difluoro-N-isopropyl-3-(4,4,5,5-tetramethyl-1,3,2-dioxaborolan-2-yl)benzamide (1.0 equiv.) at 100° C. for 15 min in the microwave to give methyl 6-(2,6-difluoro-3-(isopropylcarbamoyl)phenyl)-5-fluoropicolinate in 100% yield. LC/MS=352.9 (M+H), Rt=0.80 min. ¹H NMR (400 MHz, <cdcl3>) δ ppm 1.15-1.33 (m, 16H), 3.93-4.07 (m, 3H), 4.22-4.38 (m, 1H), 6.37-6.57 (m, 1H), 7.06-7.19 (m, 1H), 7.64-7.76 (m, 1H), 8.24 (td, J=8.80, 6.65 Hz, 1H), 8.28-8.36 (m, 1H).

### Synthesis of 6-(2,6-difluoro-3-(isopropylcarbamoyl)phenyl)-5-fluoropicolinic acid

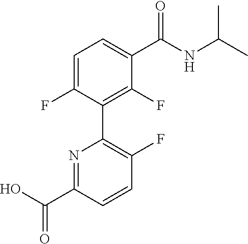 [SMILES: CC(C)NC(=O)c1ccc(F)c(-c2nc(C(=O)O)ccc2F)c1F]

Method 2 was followed using methyl 6-(2,6-difluoro-3-(isopropylcarbamoyl)phenyl)-5-fluoropicolinate (1.0 equiv.) and LiOH (2.0 equiv.) to give 6-(2,6-difluoro-3-(isopropylcarbamoyl)phenyl)-5-fluoropicolinic acid in 99% yield. LCMS (m/z): 338.9 (MH⁺), Rₜ=0.65 min.

### Synthesis of methyl 6-(2,6-difluoro-3-formylphenyl)-5-fluoropicolinate

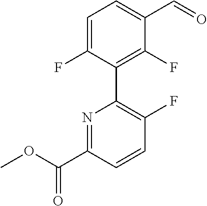 [SMILES: COC(=O)c1ccc(F)c(-c2c(F)ccc(C=O)c2F)n1]

Methyl 6-bromo-5-fluoropicolinate (1.0 equiv.) and 2,6-difluoro-3-formylphenylboronic acid (1.2 equiv.) were dissolved in THF/H₂O (10:1, 0.11 M). The mixture was degassed by bubbling argon through for 10 min. tri-tert-butylphosphine (0.5 equiv.), Pd₂(dba)₃ (0.25 equiv.), and potassium fluoride (3.3 equiv.) were added. The reaction was heated in an oil bath at 80° C. for 60 min. The cooled reaction was diluted with water and extracted with ethyl acetate. The combined organics were dried over sodium sulfate, filtered, and concentrated. The crude material was purified by flash chromatography over silica gel (heptanes/ethyl acetate gradient) to provide methyl 6-(2,6-difluoro-3-formylphenyl)-5-fluoropicolinate in 52% yield. LCMS (m/z): 296.0 (MH⁺), Rₜ=0.80 min.

### Synthesis of 6-(3-cyano-2,6-difluorophenyl)-5-fluoropicolinic acid

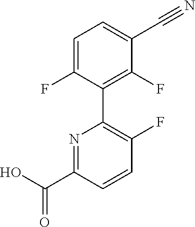 [SMILES: N#Cc1ccc(F)c(-c2nc(C(=O)O)ccc2F)c1F]

Methyl 6-(2,6-difluoro-3-formylphenyl)-5-fluoropicolinate (1.0 equiv.) and HYDROXYLAMINE HYDROCHLORIDE (2.0 equiv.) were suspended in formic acid (0.30 M). The mixture was stirred at 100° C. overnight. The cooled reaction mixture was concentrated. A 0.6M solution of aqueous sodium carbonate was added. This mixture was extracted twice with ethyl acetate. The combined aqueous layers were acidified to pH 1 with conc. HCl. The mixture was extracted twice with ethyl acetate. The combined extracts were washed twice with aqueous sodium carbonate. The organic layer was discarded. The combined aqueous layers were acidified to pH 1 with conc. HCl and extracted twice with ethyl acetate. The combined organic phases were dried over sodium sulfate, filtered, and concentrated to give 6-(3-cyano-2,6-difluorophenyl)-5-fluoropicolinic acid in 71% yield. LCMS (m/z): 279.0 (MH+), Rₜ=0.68 min.

### Synthesis of 6-(4-cyano-2-fluorophenyl)-5-fluoropicolinic acid

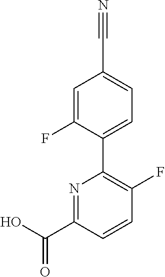 [SMILES: N#Cc1ccc(-c2nc(C(=O)O)ccc2F)c(F)c1]

To a degassed solution of 6-bromo-5-fluoropicolinic acid (1.0 equiv.) and 3-fluoro-4-(4,4,5,5-tetramethyl-1,3,2-dioxaborolan-2-yl)benzonitrile (1.5 equiv.) in DME/2M Na₂CO₃ (3:1, 0.17 M) was added (PdCl₂(dppf)-CH₂Cl₂Adduct (0.15 equiv.) The mixture was heated in the microwave at 120° C. for 30 min. The mixture was diluted with EtOAc and 1 M NaOH and separated. The organic layer was extracted with 1N NaOH. The combined aqueous was filtered through filter paper and acidified to pH 1 with 12 M HCl and extracted with EtOAc. The organic layer was dried over sodium sulfate, filtered and concentrated to yield 6-(4-cyano-2-fluorophenyl)-5-fluoropicolinic acid in 66% yield. LC/MS (m/z): 260.9 (MH+), Rt=0.69 min.

### Synthesis of 3-bromo-2,4-difluoro-N,N-dimethylbenzamide

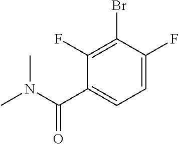

A solution of dimethylamine (1.5 equiv.), aza-HOBt (2.0 equiv.), 3-bromo-2,4-difluorobenzoic acid (1.0 equiv.) and EDC (2.0 equiv.) in DMF (0.30 M) was stirred at RT for 19 hrs. The reaction mixture was then diluted with EtOAc and water. The aqueous layer was then separated and extracted with EtOAc. The organic layer was then dried over MgSO₄ and concentrated in vaccuo to yield a white solid. The crudel was further purified by column chromatography eluting with 100% heptane to 10% EtOAc:heptane to 30% EtOAc:heptane to yield 3-bromo-2,4-difluoro-N,N-dimethylbenzamide in 85% yield. LC/MS (m/z): 265.8 (MH+), Rt=0.68 min.

### Synthesis of 2,4-difluoro-N,N-dimethyl-3-(4,4,5,5-tetramethyl-1,3,2-dioxaborolan-2-yl)benzamide

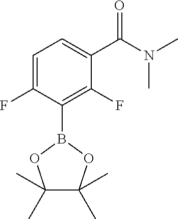 [SMILES: CN(C)C(=O)c1ccc(F)c(B2OC(C)(C)C(C)(C)O2)c1F]

A degassed solution of 3-bromo-2,4-difluoro-N,N-dimethylbenzamide (1.0 equiv.), bispinacolatoborane ester (2.0 equiv.), KOAc (2.0 equiv.), Pd₂(dba)₃ (0.045 equiv.), and tricyclohexylphosphine (0.2 equiv.) in Dioxane (0.24 M) was heated under microwave irradiation a 120° C. for 40 min. The mixture was diluted with EtOAc and water. The aqueous layer was then separated and extracted with EtOAc. The combined organics were then dried over MgSO₄ and concentrated in vacuum to yield 2,4-difluoro-N,N-dimethyl-3-(4,4,5,5-tetramethyl-1,3,2-dioxaborolan-2-yl)benzamide in 100% yield. The oil was utilised in the subsequent Suzuki coupling without further purification.

### Synthesis of methyl 6-(3-(dimethylcarbamoyl)-2,6-difluorophenyl)-5-fluoropicolinate

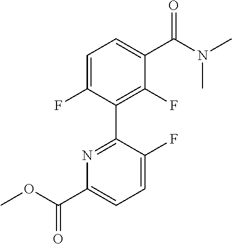

Method 5 was followed using 6-bromo-5-fluoropicolinate (1.0 equiv.) and 2,4-difluoro-N,N-dimethyl-3-(4,4,5,5-tetramethyl-1,3,2-dioxaborolan-2-yl)benzamide (1.0 equiv.) to give methyl 6-(3-(dimethylcarbamoyl)-2,6-difluorophenyl)-5-fluoropicolinate in 34% yield. LC/MS=338.9 (M+H), Rt=0.66 min.

### Synthesis of 6-(3-(dimethylcarbamoyl)-2,6-difluorophenyl)-5-fluoropicolinic acid

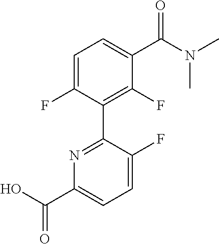 [SMILES: CN(C)C(=O)c1ccc(F)c(-c2nc(C(=O)O)ccc2F)c1F]

Method 2 was followed using methyl 6-(3-(dimethylcarbamoyl)-2,6-difluorophenyl)-5-fluoropicolinate (1.0 equiv.) and LiOH (5.5 equiv.) to give 6-(3-(dimethylcarbamoyl)-2,6-difluorophenyl)-5-fluoropicolinic acid in 100% yield. LCMS (m/z): 324.9 (MH⁺), Rₜ=0.59 min.

### Synthesis of 3-bromo-2,4-difluoro-N-methylbenzamide

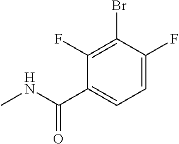

A solution of methylamine (1.5 equiv.), aza-HOBt (2.0 equiv.), 3-bromo-2,4-difluorobenzoic acid (1.0 equiv.) and EDC (2.0 equiv.) in DMF (0.30 M) was stirred at RT for 19 h. The reaction mixture was then diluted with EtOAc and water. The aqueous layer was then separated and extracted with EtOAc. The organic layer was then dried over MgSO₄ and concentrated in vaccuo to yield a white solid. The crudel was further purified by column chromatography eluting with 100% heptane to 10% EtOAc:heptane to 30% EtOAc:heptane to yield 3-bromo-2,4-difluoro-N-methylbenzamide in 92% yield. LC/MS (m/z): 249.8 (MH+), Rₜ=0.46 min.

### Synthesis of 2,4-difluoro-N-methyl-3-(4,4,5,5-tetramethyl-1,3,2-dioxaborolan-2-yl)benzamide

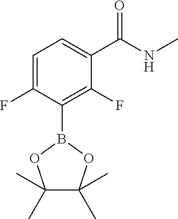 [SMILES: CNC(=O)c1ccc(F)c(B2OC(C)(C)C(C)(C)O2)c1F]

A degassed solution of 3-bromo-2,4-difluoro-N-methylbenzamide (1.0 equiv.), bispinacolatoborane ester (2.0 equiv.), KOAc (2.0 equiv.), Pd₂(dba)₃ (0.045 equiv.), and tricyclohexylphosphine (0.2 equiv.) in Dioxane (0.24 M) was heated under microwave irradiation a 120° C. for 20 min. The mixture was diluted with EtOAc and water. The aqueous layer was then separated and extracted with EtOAc. The combined organics were then dried over MgSO₄ and concentrated in vacuum to yield 2,4-difluoro-N-methyl-3-(4,4,5,5-tetramethyl-1,3,2-dioxaborolan-2-yl)benzamide in 100% yield. The oil was utilized in the subsequent Suzuki coupling without further purification.

### Synthesis of methyl 6-(2,6-difluoro-3-(methylcarbamoyl)phenyl)-5-fluoropicolinate

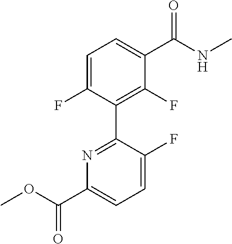

Method 5 was followed using 6-bromo-5-fluoropicolinate (1.0 equiv.) and 2,4-difluoro-N-methyl-3-(4,4,5,5-tetramethyl-1,3,2-dioxaborolan-2-yl)benzamide (1.0 equiv.) to give methyl 6-(2,6-difluoro-3-(methylcarbamoyl)phenyl)-5-fluoropicolinate in 39% yield. LC/MS=324.9 (M+H), Rt=0.63 min.

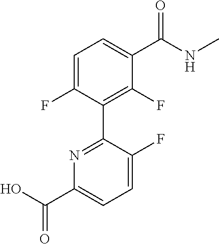 [SMILES: CNC(=O)c1ccc(F)c(-c2nc(C(=O)O)ccc2F)c1F]

Method 2 was followed using methyl 6-(2,6-difluoro-3-(methylcarbamoyl)phenyl)-5-fluoropicolinate (1.0 equiv.) and LiOH (5.5 equiv.) to give 6-(2,6-difluoro-3-(methylcarbamoyl)phenyl)-5-fluoropicolinic acid in 96% yield. LCMS (m/z): 310.9 (MH⁺), Rₜ=0.54 min.

### Synthesis of methyl 2-(4-oxopyridin-1(4H)-yl)pyrimidine-4-carboxylate

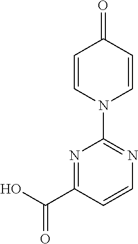 [SMILES: O=C(O)c1ccnc(-n2ccc(=O)cc2)n1]

To a solution of K₂CO₃ (3.5 equiv.), pyridin-4-ol (2.0 equiv.) and methyl 2-chloropyrimidine-4-carboxylate (1.0 equiv.) in H₂O (0.80 M) was heated at 95° C. in microwave for 15 min. Add 1 M HCl to acidify and observe ppt. Centrifuge and remove soluble portion by pipette. Stir in dilute aq HCl, centrifuge and remove the aqueous layer by pipette. Add EtOAc and THF. Centrifuge and remove liquid by pipette. Dry under high vacuum to give 2-(4-oxopyridin-1(4H)-yl)pyrimidine-4-carboxylic acid in 100% yield. LCMS (m/z): 218.0 (MH+), Rt=0.32 min.

### Synthesis of 5-fluoro-6-phenylpicolinic acid

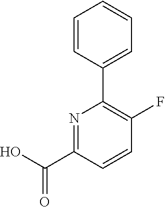 [SMILES: O=C(O)c1ccc(F)c(-c2ccccc2)n1]

To methyl 6-bromo-5-fluoropicolinate (1.0 equiv.) in DME (0.13 M) in a microwave vial add phenylboronic acid (1.5 equiv.) and Na₂CO₃ (7.5 equiv.). Flush with N₂ and add Pd(PPh₃)₄ (0.05 equiv.). Microwave heat at 120° C. for 35 min. DME soluble portion was dried over Na₂SO₄, concentrated and triturated with several drops EtOAc. Filter. Dry solid on high vacuum to give 5-fluoro-6-phenylpicolinic acid in 100% yield. LCMS (m/z): 218.0 (MH+), Rt=0.69 min.

### Synthesis of 5-fluoro-6-(4-(methylsulfonyl)phenyl)picolinic acid

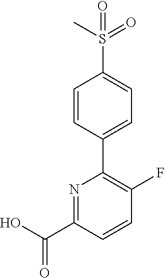 [SMILES: CS(=O)(=O)c1ccc(-c2nc(C(=O)O)ccc2F)cc1]

To methyl 6-bromo-5-fluoropicolinate (1.0 equiv.) in DME (0.13 M) in a microwave vial add 4-(methylsulfonyl)phenylboronic acid (1.5 equiv.) and Na₂CO₃ (7.5 equiv.). Flush with N₂ and add Pd(PPh₃)₄ (0.05 equiv.). Microwave heat at 120° C. for 35 min. DME soluble portion was dried over Na₂SO₄, concentrated and triturated with several drops EtOAc. Filter. Dry solid on high vacuum to give 5-fluoro-6-(4-(methylsulfonyl)phenyl)picolinic acid in 100% yield. LCMS (m/z): 296.0 (MH+), Rt=0.55 min.

### Synthesis of 2-chloro-6-phenylpyrazine

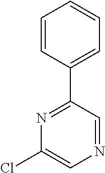 [SMILES: Clc1cncc(-c2ccccc2)n1]

To a degassed mixture of dichloro pyrazine (2.0 equiv.), phenylboronic acid (1.0 equiv.) in DME (0.25 M) and 2 M Na₂CO₃ (1.0 equiv.) was added PdCl₂(dppf).CH₂Cl₂ adduct (0.1 equiv.) (98 mg, 2.442 mmol). The reaction mixture was heated in microwave at 120° C. for 15 min. The reaction mixture was partitioned between ethyl acetate and sat. aq. sodium bicarbonate then the organic layer was washed with brine. The organic layer was separated dried with MgSO₄, filtered and concentrated. The crude was purified by isco with heptanes to 30% EtOAc, to yield 2-chloro-6-phenylpyrazine in 74% yield. LCMS (m/z): 191.0 (MH+), Rt=1.00 min.

### Synthesis of methyl 6-phenylpyrazine-2-carboxylate

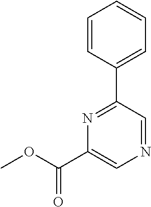 [SMILES: COC(=O)c1cncc(-c2ccccc2)n1]

To a steel pressure vessel with stir bar was added 2-chloro-6-phenylpyrazine (1.0 equiv.), MeOH (0.2 M), TRIETHYLAMINE (1.0 equiv.). Nitrogen was bubbled through the solution for 5 min then Pd (II) (R)-Binap (0.1 equiv.) was added. Vessel sealed and Carbon Monoxide (1.0 equiv.) atmosphere was inserted to 70 psi. The reaction mixture was then placed in an oil bath and heated to 100° C. for 18 hrs. The mixture was diluted with water and extracted with EtOAc. Organics combined, washed with brine, dried (Na₂SO₄), filtered and concentrated. The crude was purified by flash chromatography (0-20% EtOAc:Heptanes) to yield methyl 6-phenylpyrazine-2-carboxylate, obtained in 99% yield. LCMS (m/z): 215.0 (MH⁺), Rₜ=0.81 min.

### Synthesis of 6-phenylpyrazine-2-carboxylic acid

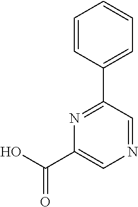 [SMILES: O=C(O)c1cncc(-c2ccccc2)n1]

Method 2 was followed using methyl 6-phenylpyrazine-2-carboxylate (1.0 equiv.) and LiOH (2.0 equiv.) to give 6-phenylpyrazine-2-carboxylic acid in 67% yield. LCMS (m/z): 201.0 (MH⁺), Rₜ=0.63 min.

### Synthesis of 5-(1-hydroxy-2-phenylethylidene)-2,2-dimethyl-1,3-dioxane-4,6-dione

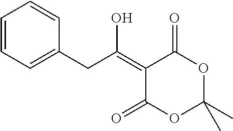 [SMILES: CC1(C)OC(=O)C(=C(O)Cc2ccccc2)C(=O)O1]

To a solution of Meldrum's acid (0.98 equiv.) in DCM (0.87 M) cooled to 0° C. was added pyridine (2.70 equiv.) followed by 2-phenylacetyl chloride (1.0 equiv.). The resulting mixture was stirred and allowed to warm to room temperature over 4 h. After this time the reaction mixture was diluted with DCM (2.8× initial reaction solvent volume) and 1 N HCl (2.3× initial reaction solvent volume). The organic layer was separated then washed further with 1 N HCl (0.6× initial solvent volume) then brine and dried over Na₂SO₄, filtered, and concentrated in vacuo to yield the desired product 5-(1-hydroxy-2-phenylethylidene)-2,2-dimethyl-1,3-dioxane-4,6-dione as a oil (crude mass recovery=98% yield). The material was used without further purification.

### Synthesis of ethyl 3-oxo-4-phenylbutanoate

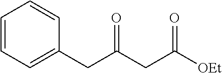 [SMILES: CCOC(=O)CC(=O)Cc1ccccc1]

A solution of unpurified 5-(1-hydroxy-2-phenylethylidene)-2,2-dimethyl-1,3-dioxane-4,6-dione (1.00 equiv.) in EtOH (0.74 M) was heated to reflux (85° C.) for 16 h. The resulting mixture was cooled to room temperature and concentrated in vacuo to yield a dark purple oil. The oil was further purified by flash column chromatography by ISCO Combi-flash Rf system with a Redisep column eluting with 0-20% EtOAc/heptanes to afford the desired product ethyl 3-oxo-4-phenylbutanoate as a yellow oil (30% yield over two steps). LC/MS (m/z): 207.0 (MH⁺), Rₜ=0.82 min. ¹H NMR (400 MHz, CHLOROFORM-d) δ ppm 1.27 (t, 3H), 3.45 (s, 2H), 3.83 (s, 2H), 4.17 (q, 2H), 7.19-7.38 (m, 5H).

### Synthesis of ethyl 4-oxo-5-phenyl-1,4-dihydropyridine-3-carboxylate

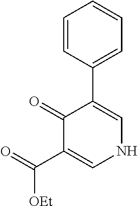 [SMILES: CCOC(=O)c1c[nH]cc(-c2ccccc2)c1=O]

To a solution of ethyl 3-oxo-4-phenylbutanoate (1.00 equiv.) in EtOH (0.45 M) under argon at room temperature was added 1,3,5-triazine (1.05 equiv.) followed by dropwise addition of a solution of sodium ethanoate (1.05 equiv., 2.68 M in EtOH). The resulting mixture was then heated to reflux (85° C.) and stirred at reflux for 2 h. The resulting mixture was cooled to room temperature and the volatiles were removed by concentration in vacuo. To the resulting concentrate was added 1N HCl (1× initial solvent reaction volume) causing the formation of a yellow precipitate. The precipitate was collected by vacuum filtration then washed sequentially with water followed by EtOAc. The solid was further dried by high vacuum for 20 h affording the desired product ethyl 4-oxo-5-phenyl-1,4-dihydropyridine-3-carboxylate as a yellow solid (55% yield). LC/MS (m/z): 244.1 (MH⁺), Rₜ=0.58 min. ¹H NMR (400 MHz, DMSO-d) δ ppm 1.25 (t, 3H), 4.27 (q, 2H), 7.23-7.42 (m, 3H), 7.51-7.59 (m, 2H), 7.80 (d, 1H), 8.16 (d, 1H), 11.88 (broad s, 1H).

### Synthesis of 4-oxo-5-phenyl-1,4-dihydropyridine-3-carboxylic acid

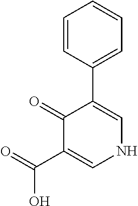 [SMILES: O=C(O)c1c[nH]cc(-c2ccccc2)c1=O]

To a solution ethyl 4-oxo-5-phenyl-1,4-dihydropyridine-3-carboxylate (1.00 equiv.) in MeOH (2.3 M) at room temperature was added 2N NaOH (3.40 equiv.). The resulting mixture was then heated to reflux (60° C.) and stirred at reflux for 2 h. The resulting mixture was cooled to room temperature and then poured into 2 N HCl (6× initial solvent reaction volume) causing the formation of an off-white precipitate. The precipitate was collected by vacuum filtration then washed sequentially with water followed by EtOAc. The solid was further dried by high vacuum for 20 h affording the desired product 4-oxo-5-phenyl-1,4-dihydropyridine-3-carboxylic acid as an off-white solid (98% yield). LC/MS (m/z): 216.0 (MH⁺), Rₜ=0.54 min. ¹H NMR (400 MHz, DMSO-d) δ ppm) 7.35-7.46 (m, 3H), 7.62-7.66 (m, 2H), 8.22 (d, 1H), 8.59 (d, 1H), 13.11 (broad s, 1H).

### Synthesis of benzyl 1-benzyl-4-nitro-1H-pyrazole-3-carboxylate

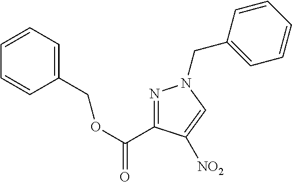 [SMILES: O=C(OCc1ccccc1)c1nn(Cc2ccccc2)cc1[N+](=O)[O-]]

To a solution of 4-nitro-1H-pyrazole-3-carboxylic acid (1.0 equiv.) in DMF (0.3 M) was added BENZYL BROMIDE (2.0 equiv.) and Cs₂CO₃ (4.0 equiv.) at room temperature. The reaction mixture was stirred at room temperature for 1.5 h. After quenched with H₂O, The reaction mixture was extracted with EtOAc. The combined organic layer was washed with water and brine, and dried over anhydrous sodium sulfate, filtered and concentrated in vacuo. The benzyl 1-benzyl-1H-pyrazole-3-carboxylate was obtained as a colorless oil by flash column chromatography (20% EtOAc in Hexanes) in 51% yield. LCMS (m/z): 338.2 (MH⁺), Rₜ=1.02 min. ¹H NMR (400 M Hz, CHLOROFORM-d) δ ppm 7.98 (s, 1H), 7.49-7.34 (m, 8H), 7.31 (dd, J=6.7, 2.7 Hz, 2H), 5.43 (s, 2H), 5.33 (s, 2H).

### Synthesis of 1-benzyl-4-nitro-1H-pyrazole-3-carboxylic acid

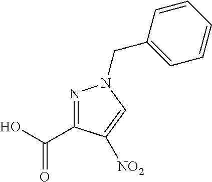 [SMILES: O=C(O)c1nn(Cc2ccccc2)cc1[N+](=O)[O-]]

To a solution of benzyl 1-benzyl-4-nitro-1H-pyrazole-3-carboxylate (1.0 equiv.) in MeOH:THF (1:1, 0.3M) was added LiOH (1.0 Min H₂O) (2.0 equiv.) at room temperature. The reaction mixture was stirred at room temperature for 1 h, the reaction mixture was adjusted to pH=4-5 by 1N HCl, the reaction mixture was then extracted with EtOAc 3 times. The combined organic layer was washed with water and brine, and dried over anhydrous sodium sulfate, filtered and concentrated in vacuo. The crude material was recrystallized from Et₂O to remove benzyl alcohol. LCMS (m/z): 248.0 (MH⁺), Rₜ=0.65 min. ¹H NMR (400 M Hz, CHLOROFORM-d) δ ppm 8.10 (s, 1H), 7.47-7.42 (m, 3H), 7.39-7.32 (m, 3H), 5.40 (s, 2H).

### Synthesis of 1-benzyl-N-(4-((2R,4R,5S,6R)-4,5-dihydroxy-5,6-dimethyltetrahydro-2H-pyran-2-yl)pyridin-3-yl)-4-nitro-1H-pyrazole-3-carboxamide

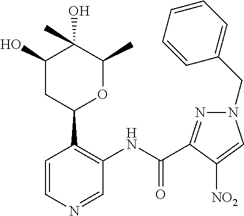 [SMILES: C[C@H]1O[C@@H](c2ccncc2NC(=O)c2nn(Cc3ccccc3)cc2[N+](=O)[O-])C[C@@H](O)[C@]1(C)O]

A solution of (2R,3S,4R,6R)-6-(3-aminopyridin-4-yl)-2,3-dimethyltetrahydro-2H-pyran-3,4-diol (1.0 equiv) and 1-benzyl-4-nitro-1H-pyrazole-3-carboxylic acid (1.1 equiv.), HOAT (1.2 equiv.) and EDC (1.2 equiv.) in DMF (0.5 M) was stirred for 12 hours at room temperature. The reaction mixture was partitioned between EtOAc and NaHCO₃, the organic was washed by water and brine, dried over anhydrous sodium sulfate, filtered and concentrated in vacuo to give 1-benzyl-N-(4-((2R,4R,5S,6R)-4,5-dihydroxy-5,6-dimethyltetrahydro-2H-pyran-2-yl)pyridin-3-yl)-4-nitro-1H-pyrazole-3-carboxamide in 99% yield. LCMS (m/z): 468.1 (MH⁺), Rt=0.57 min.

### Synthesis of 4-amino-1-benzyl-N-(4-((2R,4R,5S,6R)-4,5-dihydroxy-5,6-dimethyltetrahydro-2H-pyran-2-yl)pyridin-3-yl)-1H-pyrazole-3-carboxamide

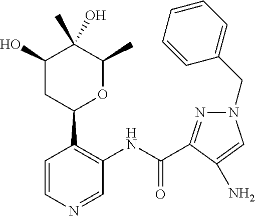 [SMILES: C[C@H]1O[C@@H](c2ccncc2NC(=O)c2nn(Cc3ccccc3)cc2N)C[C@@H](O)[C@]1(C)O]

A solution of 1-benzyl-N-(4-((2R,4R,5S,6R)-4,5-dihydroxy-5,6-dimethyltetrahydro-2H-pyran-2-yl)pyridin-3-yl)-4-nitro-1H-pyrazole-3-carboxamide (1.0 equiv.) in methanol (0.3 M) was degassed by nitrogen for 10 minutes, 10% Pd/C (0.2 equiv.) was added. The reaction mixture was stirred at room temperature for 1 h under hydrogen balloon. The reaction mixture was filtered through celite and washed by MeOH and EtOAc, the filtrate was concentrated in vacuo, the crude material was purified by reverse phase HPLC, the pure fraction was combined and lyophilized to give the TFA salt of 4-amino-1-benzyl-N-(4-((2R,4R,5S,6R)-4,5-dihydroxy-5,6-dimethyltetrahydro-2H-pyran-2-yl)pyridin-3-yl)-1H-pyrazole-3-carboxamide. LCMS (m/z): 438.2 (MH⁺), Rₜ=0.46 min. ¹H NMR (400 M Hz, DMSO-d6) δ ppm 9.25 (s, 1H), 8.35 (d, J=5.1 Hz, 1H), 7.57 (br. s., 1H), 7.41 (d, J=5.1 Hz, 1H), 7.37-7.30 (m, 3H), 7.30-7.27 (m, 1H), 7.23-7.19 (m, 2H), 5.38-5.26 (m, 2H), 4.74 (dd, J=11.7, 2.0 Hz, 1H), 3.50 (m, 1H), 3.36 (m, 1H), 1.83-1.98 (m, 1H), 1.54 (q, J=11.9 Hz, 1H), 1.19 (d, J=6.7 Hz, 3H), 0.95 (s, 3H).

### Synthesis of tert-butyl ((2R,3S,4R,6R)-6-(3-(6-(2,6-difluoro-3-methoxyphenyl)-5-fluoropicolinamido)pyridin-4-yl)-3-hydroxy-2,3-dimethyltetrahydro-2H-pyran-4-yl)carbamate

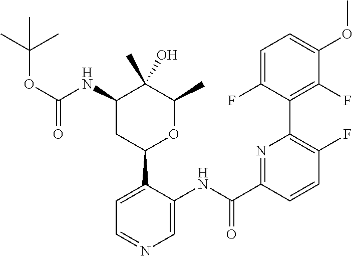 [SMILES: COc1ccc(F)c(-c2nc(C(=O)Nc3cnccc3[C@H]3C[C@@H](NC(=O)OC(C)(C)C)[C@](C)(O)[C@@H](C)O3)ccc2F)c1F]

Method 5 was followed using tert-butyl ((2R,3S,4R,6R)-6-(3-(6-bromo-5-fluoropicolinamido)pyridin-4-yl)-3-hydroxy-2,3-dimethyltetrahydro-2H-pyran-4-yl)carbamate (1.0 equiv.) and (2,6-difluoro-3-methoxyphenyl)boronic acid (2.5 equiv.) at 100° C. for 15 min in the microwave to give tert-butyl ((2R,3S,4R,6R)-6-(3-(6-(2,6-difluoro-3-methoxyphenyl)-5-fluoropicolinamido)pyridin-4-yl)-3-hydroxy-2,3-dimethyltetrahydro-2H-pyran-4-yl)carbamate in 92% yield. LC/MS=603.2 (M+H), Rt=0.84 min.

### Synthesis of tert-butyl ((2R,3S,4R,6R)-6-(3-(6-(1,1-dioxidothiomorpholino)-5-fluoropicolinamido)pyridin-4-yl)-3-hydroxy-2,3-dimethyltetrahydro-2H-pyran-4-yl)carbamate

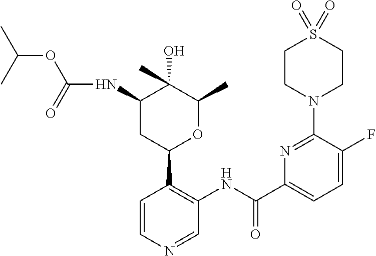 [SMILES: CC(C)OC(=O)N[C@@H]1C[C@H](c2ccncc2NC(=O)c2ccc(F)c(N3CCS(=O)(=O)CC3)n2)O[C@H](C)[C@@]1(C)O]

To a mixture of tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.), thiomorpholine 1,1-dioxide (1.2 equiv.), cesium carbonate (2.0 equiv.) and (9,9-dimethyl-9H-xanthene-4,5-diyl)bis(diphenylphosphine) (0.1 equiv.) in dioxane purged with nitrogen was added Pd₂(dba)₃ (0.1 equiv.). The mixture was heated at 115° C. in microwave for 40 mins. The reaction was cooled off to rt, diluted with EtOAc and washed with water. Wash the organic layer with brine and dry it over Na₂SO₄. Concentrate to give tert-butyl ((2R,3S,4R,6R)-6-(3-(6-(1,1-dioxidothiomorpholino)-5-fluoropicolinamido)pyridin-4-yl)-3-hydroxy-2,3-dimethyltetrahydro-2H-pyran-4-yl)carbamate in 100% yield. LCMS (m/z): 594.0 (MH+), Rt=0.64 min.

### Synthesis of methyl 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoate

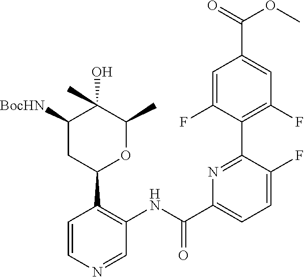 [SMILES: COC(=O)c1cc(F)c(-c2nc(C(=O)Nc3cnccc3[C@H]3C[C@@H](NC(=O)OC(C)(C)C)[C@](C)(O)[C@@H](C)O3)ccc2F)c(F)c1]

Method 5 was followed using tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.) and methyl 3,5-difluoro-4-(4,4,5,5-tetramethyl-1,3,2-dioxaborolan-2-yl)benzoate (2.5 equiv.) at 100° C. for 20 mins in the microwave to give methyl 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoate in 100% yield. LC/MS=631.2 (M+H), Rt=0.89 min.

### Synthesis of 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoic acid

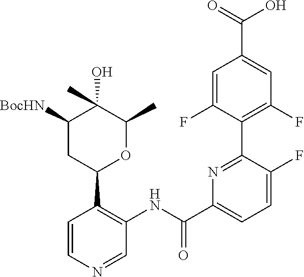 [SMILES: C[C@H]1O[C@@H](c2ccncc2NC(=O)c2ccc(F)c(-c3c(F)cc(C(=O)O)cc3F)n2)C[C@@H](NC(=O)OC(C)(C)C)[C@]1(C)O]

Method 2 was followed using methyl 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoate (1.0 equiv.) and LiOH (2.0 equiv.) to give 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoic acid in 31% yield. LCMS (m/z): 617.0 (MH⁺), Rₜ=0.75 min.

### Synthesis of tert-butyl (2R,3S,4R,6R)-6-(3-(6-(2,6-difluoro-4-(methylcarbamoyl)phenyl)-5-fluoropicolinamido)pyridin-4-yl)-3-hydroxy-2,3-dimethyltetrahydro-2H-pyran-4-ylcarbamate

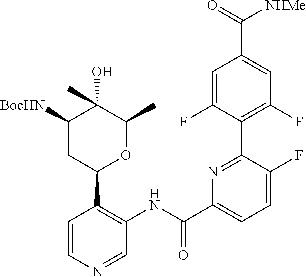 [SMILES: CNC(=O)c1cc(F)c(-c2nc(C(=O)Nc3cnccc3[C@H]3C[C@@H](NC(=O)OC(C)(C)C)[C@](C)(O)[C@@H](C)O3)ccc2F)c(F)c1]

To 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoic acid (1.0 equiv.), methanamine hydrochloride (1.5 equiv.) and N-ethyl-N-isopropylpropan-2-amine (1.4 equiv.) in DMF (0.10 M) was added 3H-[1,2,3]triazolo[4,5-b]pyridin-3-ol (2.0 equiv.) and N1-((ethylimino)methylene)-N3,N3-dimethylpropane-1,3-diamine hydrochloride (2.0 equiv.). The mixture was stirred at rt for 16 hrs. Add water and extract with EtOAc. Wash the organic layer with brine and dry it over Na₂SO₄. Filter and concentrate to yield tert-butyl (2R,3S,4R,6R)-6-(3-(6-(2,6-difluoro-4-(methylcarbamoyl)phenyl)-5-fluoropicolinamido)pyridin-4-yl)-3-hydroxy-2,3-dimethyltetrahydro-2H-pyran-4-ylcarbamate in 100% yield. LCMS (m/z): 629.9 (MH+), Rt=0.69 min.

### Synthesis of tert-butyl (2R,3S,4R,6R)-6-(3-(6-(4-(dimethylcarbamoyl)-2,6-difluorophenyl)-5-fluoropicolinamido)pyridin-4-yl)-3-hydroxy-2,3-dimethyltetrahydro-2H-pyran-4-ylcarbamate

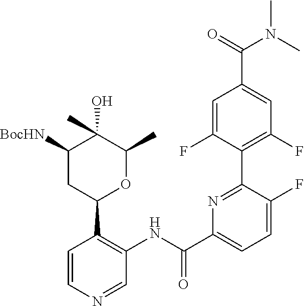 [SMILES: C[C@H]1O[C@@H](c2ccncc2NC(=O)c2ccc(F)c(-c3c(F)cc(C(=O)N(C)C)cc3F)n2)C[C@@H](NC(=O)OC(C)(C)C)[C@]1(C)O]

To 4-(6-(4-((2R,4R,5S,6R)-4-(tert-butoxycarbonylamino)-5-hydroxy-5,6-dimethyltetrahydro-2H-pyran-2-yl)pyridin-3-ylcarbamoyl)-3-fluoropyridin-2-yl)-3,5-difluorobenzoic acid (1.0 equiv.), dimethylamine (1.0 equiv.) and N-ethyl-N-isopropylpropan-2-amine (1.0 equiv.) in DMF (0.10 M) was added 3H-[1,2,3]triazolo[4,5-b]pyridin-3-ol (2.0 equiv.) and N1-((ethylimino)methylene)-N3,N3-dimethylpropane-1,3-diamine hydrochloride (2.0 equiv.). The mixture was stirred at rt for 16 hrs. Add water and extract with EtOAc. Wash the organic layer with brine and dry it over Na₂SO₄. Filter and concentrate to yield tert-butyl (2R,3S,4R,6R)-6-(3-(6-(4-(dimethylcarbamoyl)-2,6-difluorophenyl)-5-fluoropicolinamido)pyridin-4-yl)-3-hydroxy-2,3-dimethyltetrahydro-2H-pyran-4-ylcarbamate in 100% yield. LCMS (m/z): 644.1 (MH+), Rt=0.76 min.

### Synthesis of 2-(2,6-difluoro-3-(methylthio)phenyl)-4,4,5,5-tetramethyl-1,3,2-dioxaborolane

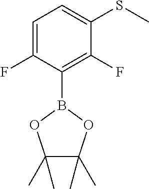 [SMILES: CSc1ccc(F)c(B2OC(C)(C)C(C)(C)O2)c1F]

To a solution of (2,4-difluorophenyl)(methyl)sulfane (1.0 equiv.) in dry THF (0.2M) under an atmosphere of N₂ at −78° C. was added n-butyllithium (1.3 equiv., 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.2 equiv.). The reaction was allowed to warm to room temperature. Upon completion, the reaction was quenched with NaHCO_{3 (sat.)} and extracted with EtOAc. The organics were washed with brine, dried over Na₂SO₄, filtered and concentrated to yield 2-(2,6-difluoro-3-(methylthio)phenyl)-4,4,5,5-tetramethyl-1,3,2-dioxaborolane in 81%. ¹H NMR (400 MHz, <cdcl3>) δ ppm 1.34-1.37 (m, 12H), 2.38 (s, 3H), 6.79 (t, J=8.41 Hz, 1H), 7.31 (d, J=6.26 Hz, 1H).

### Synthesis of tert-butyl (2R,3S,4R,6R)-6-(3-(6-(2,6-difluoro-3-(methylthio)phenyl)-5-fluoropicolinamido)pyridin-4-yl)-3-hydroxy-2,3-dimethyltetrahydro-2H-pyran-4-ylcarbamate

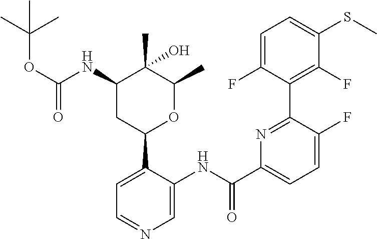 [SMILES: CSc1ccc(F)c(-c2nc(C(=O)Nc3cnccc3[C@H]3C[C@@H](NC(=O)OC(C)(C)C)[C@](C)(O)[C@@H](C)O3)ccc2F)c1F]

Method 5 was followed using 2-(2,6-difluoro-3-(methylthio)phenyl)-4,4,5,5-tetramethyl-1,3,2-dioxaborolane (2.5 equiv.) and tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.) at 100° C. for 30 min in the microwave to give tert-butyl (2R,3S,4R,6R)-6-(3-(6-(2,6-difluoro-3-(methylthio)phenyl)-5-fluoropicolinamido)pyridin-4-yl)-3-hydroxy-2,3-dimethyltetrahydro-2H-pyran-4-ylcarbamate in 80% yield. LC/MS=619.1 (M+H), Rt=0.88 min.

Synthesis of 6-fluoro-5-(4,4,5,5-tetramethyl-1,3,2-dioxaborolan-2-yl)pyridin-2-amine

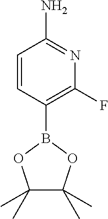 [SMILES: CC1(C)OB(c2ccc(N)nc2F)OC1(C)C]

To a suspension of 5-bromo-6-fluoropyridin-2-amine (1.0 equiv.), BIS(PINACOLATO)DIBORON (1.5 equiv.), potassium acetate (3.0 equiv.) in Dioxane (0.27 M) was added PdCl₂(dppf) (0.1 equiv.). The solution was submitted to microwave heating at 110° C. for 20 minutes. The reaction was filtered through a 1 uM HPLC frit, rinsing with additional EtOAc and the volatiles were removed in vacuo to give 6-fluoro-5-(4,4,5,5-tetramethyl-1,3,2-dioxaborolan-2-yl)pyridin-2-amine. The crude material was taken on directly to next step.

### Synthesis of tert-butyl ((2R,3S,4R,6R)-6-(3-(6′-amino-2′,3-difluoro-[2,3′-bipyridine]-6-carboxamido)pyridin-4-yl)-3-hydroxy-2,3-dimethyltetrahydro-2H-pyran-4-yl)carbamate

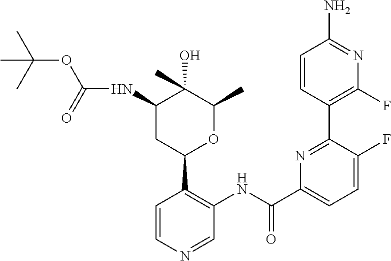 [SMILES: C[C@H]1O[C@@H](c2ccncc2NC(=O)c2ccc(F)c(-c3ccc(N)nc3F)n2)C[C@@H](NC(=O)OC(C)(C)C)[C@]1(C)O]

To a suspension of tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.), 6-fluoro-5-(4,4,5,5-tetramethyl-1,3,2-dioxaborolan-2-yl)pyridin-2-amine (1.5 equiv.), sodium carbonate (2.0 equiv.) in DME (0.18 M) was added Pd(Ph₃P)₄ (0.05 equiv.). The solution was submitted to microwave heating at 120° C. for 30 minutes. The reaction was filtered through a 1 uM HPLC frit, rinsing with additional EtOAc and the volatiles were removed in vacuo to give tert-butyl (2R,3S,4R,6R)-6-(3-(6′-amino-2′,3-difluoro-2,3′-bipyridine-6-carboxamido)pyridin-4-yl)-3-hydroxy-2,3-dimethyltetrahydro-2H-pyran-4-ylcarbamate. The crude material was taken on directly to next step. LCMS (m/z): 571.0 (MH+), Rt=0.68 min.

### Synthesis of 5-fluoro-6-(1H-pyrrolo[2,3-b]pyridin-5-yl)picolinic acid

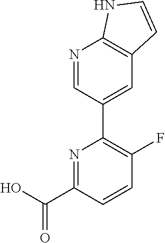 [SMILES: O=C(O)c1ccc(F)c(-c2cnc3[nH]ccc3c2)n1]

To a suspension of methyl 6-bromo-5-fluoropicolinate (1.0 equiv.), 5-(4,4,5,5-tetramethyl-1,3,2-dioxaborolan-2-yl)-1H-pyrrolo[2,3-b]pyridine (1.5 equiv.), sodium carbonate (7.5 equiv.) in DME (0.13 M) was added Pd(Ph₃P)₄ (0.05 equiv.). The solution was submitted to microwave heating at 120° C. for 15 minutes. The reaction mixture was left at rt for two weeks. DME soluble portion was removed via pipette and then dried with Na₂SO₄. After concentration, triturate with few drops ethyl acetate. Discard organic soluble portion. Remaining solid was used as is in next step to give 5-fluoro-6-(1H-pyrrolo[2,3-b]pyridin-5-yl)picolinic acid. LCMS (m/z): 258.0 (MH⁺), Rₜ=0.47 min.

### Synthesis of N-(4-bromo-3,5-difluorophenyl)acetamide

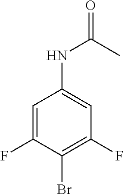

To 4-bromo-3,5-difluoroaniline (1.0 equiv.) in THF (0.1 M) at rt was added acetyl chloride (1.8 equiv.) and then N-ethyl-N-isopropylpropan-2-amine (2.5 equiv.). After stirred at rt for 2 hrs, the reaction mixture was concentrated, quenched with H₂O and extracted with EtOAc. The organic layer was washed with Brine, dried over Na₂SO₄ and concentrated to give N-(4-bromo-3,5-difluorophenyl)acetamide in 100% yield. LC/MS=249.8 (M+H), Rt=0.73 min.

### Synthesis of N-(3,5-difluoro-4-(4,4,5,5-tetramethyl-1,3,2-dioxaborolan-2-yl)phenyl)acetamide

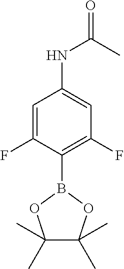 [SMILES: CC(=O)Nc1cc(F)c(B2OC(C)(C)C(C)(C)O2)c(F)c1]

To a suspension of triscyclohexylphospine (0.2 equiv.), N-(4-bromo-3,5-difluorophenyl)acetamide (1.0 equiv.), BIS(PINACOLATO)DIBORON (2.0 equiv.), POTASSIUM ACETATE (2.0 equiv.) in Dioxane (0.24 M) was added TRIS(DIBENZYLIDENEACETONE)DIPALLADIUM(0) (0.1 equiv.). The solution was heated at 110° C. for 16 hrs. The reaction was filtered through a HPLC frit, rinsing with additional EtOAc and the volatiles were removed in vacuo to give N-(3,5-difluoro-4-(4,4,5,5-tetramethyl-1,3,2-dioxaborolan-2-yl)phenyl)acetamide. The crude material was taken on directly to next step.

### Synthesis of tert-butyl (2R,3S,4R,6R)-6-(3-(6-(4-acetamido-2,6-difluorophenyl)-5-fluoropicolinamido)pyridin-4-yl)-3-hydroxy-2,3-dimethyltetrahydro-2H-pyran-4-ylcarbamate

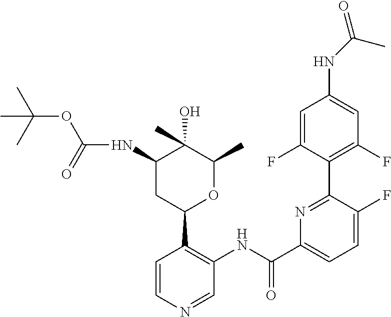 [SMILES: CC(=O)Nc1cc(F)c(-c2nc(C(=O)Nc3cnccc3[C@H]3C[C@@H](NC(=O)OC(C)(C)C)[C@](C)(O)[C@@H](C)O3)ccc2F)c(F)c1]

Method 5 was followed using tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.) and N-(3,5-difluoro-4-(4,4,5,5-tetramethyl-1,3,2-dioxaborolan-2-yl)phenyl)acetamide (2.5 equiv.) to give tert-butyl (2R,3S,4R,6R)-6-(3-(6-(4-acetamido-2,6-difluorophenyl)-5-fluoropicolinamido)pyridin-4-yl)-3-hydroxy-2,3-dimethyltetrahydro-2H-pyran-4-ylcarbamate in 100% yield. LC/MS=630.1 (M+H), Rt=0.78 min.

### Synthesis of N-(4-bromo-3,5-difluorophenyl)isobutyramide

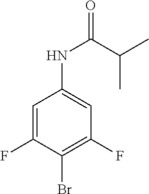

To 4-bromo-3,5-difluoroaniline (1.0 equiv.) in THF (0.1 M) at rt was added isobutyryl chloride (1.8 equiv.) and then N-ethyl-N-isopropylpropan-2-amine (2.5 equiv.). After stirred at rt for 2 hrs, the reaction mixture was concentrated, quenched with H₂O and extracted with EtOAc. The organic layer was washed with Brine, dried over Na₂SO₄ and concentrated to give N-(4-bromo-3,5-difluorophenyl)isobutyramide in 100% yield. LC/MS=277.9 (M+H), Rt=0.87 min.

### Synthesis of N-(3,5-difluoro-4-(4,4,5,5-tetramethyl-1,3,2-dioxaborolan-2-yl)phenyl)isobutyramide

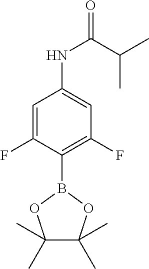 [SMILES: CC(C)C(=O)Nc1cc(F)c(B2OC(C)(C)C(C)(C)O2)c(F)c1]

To a suspension of triscyclohexylphospine (0.2 equiv.), N-(4-bromo-3,5-difluorophenyl)isobutyramide (1.0 equiv.), BIS(PINACOLATO)DIBORON (2.0 equiv.), POTASSIUM ACETATE (2.0 equiv.) in Dioxane (0.24 M) was added TRIS(DIBENZYLIDENEACETONE)DIPALLADIUM(0) (0.1 equiv.). The solution was heated at 110° C. for 16 hrs. The reaction was filtered through a HPLC frit, rinsing with additional EtOAc and the volatiles were removed in vacuo to give N-(3,5-difluoro-4-(4,4,5,5-tetramethyl-1,3,2-dioxaborolan-2-yl)phenyl)isobutyramide. The crude material was taken on directly to next step.

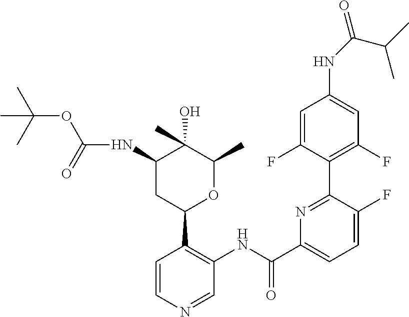 [SMILES: CC(C)C(=O)Nc1cc(F)c(-c2nc(C(=O)Nc3cnccc3[C@H]3C[C@@H](NC(=O)OC(C)(C)C)[C@](C)(O)[C@@H](C)O3)ccc2F)c(F)c1]

Method 5 was followed using tert-butyl (2R,3S,4R,6R)-6-(3-(6-bromo-5-fluoropicolinamido)pyridin-4-yl)-3-hydroxy-2,3-dimethyltetrahydro-2H-pyran-4-ylcarbamate (1.0 equiv.) and N-(3,5-difluoro-4-(4,4,5,5-tetramethyl-1,3,2-dioxaborolan-2-yl)phenyl)isobutyramide (2.5 equiv.) to give tert-butyl (2R,3S,4R,6R)-6-(3-(6-(2,6-difluoro-4-isobutyramidophenyl)-5-fluoropicolinamido)pyridin-4-yl)-3-hydroxy-2,3-dimethyltetrahydro-2H-pyran-4-ylcarbamate in 100% yield. LC/MS=658.3 (M+H), Rt=0.85 min.

### Synthesis of 3-amino-6-(1,5-dimethyl-1H-pyrazol-4-yl)-N-(4-((2R,4R,5S,6R)-5-ethyl-4,5-dihydroxy-6-methyltetrahydro-2H-pyran-2-yl)pyridin-3-yl)picolinamide

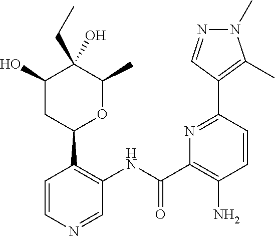 [SMILES: CC[C@]1(O)[C@H](O)C[C@H](c2ccncc2NC(=O)c2nc(-c3cnn(C)c3C)ccc2N)O[C@@H]1C]

Method 1 was followed using (2R,3R,4R,6R)-6-(3-(3-amino-6-bromopicolinamido)pyridin-4-yl)-3-ethyl-3-hydroxy-2-methyltetrahydro-2H-pyran-4-yl acetate (1.0 equiv.) and 1,5-dimethyl-4-(4,4,5,5-tetramethyl-1,3,2-dioxaborolan-2-yl)-1H-pyrazole (2.0 equiv.) to give 3-amino-6-(1,5-dimethyl-1H-pyrazol-4-yl)-N-(4-((2R,4R,5S,6R)-5-ethyl-4,5-dihydroxy-6-methyltetrahydro-2H-pyran-2-yl)pyridin-3-yl)picolinamide in 30% yield. LC/MS=467.2 (M+H), Rt=0.49 min.

### Synthesis of (2R,3R,4R,6R)-6-(3-(3-amino-6-(4,4,5,5-tetramethyl-1,3,2-dioxaborolan-2-yl)picolinamido)pyridin-4-yl)-3-ethyl-3-hydroxy-2-methyltetrahydro-2H-pyran-4-yl acetate

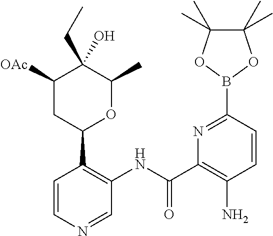 [SMILES: CC[C@@]1(O)[C@@H](C)O[C@@H](c2ccncc2NC(=O)c2nc(B3OC(C)(C)C(C)(C)O3)ccc2N)C[C@H]1OC(C)=O]

To a suspension of triscyclohexylphospine (0.7 equiv.), (2R,3R,4R,6R)-6-(3-(3-amino-6-bromopicolinamido)pyridin-4-yl)-3-ethyl-3-hydroxy-2-methyltetrahydro-2H-pyran-4-yl acetate (1.0 equiv.), BIS(PINACOLATO)DIBORON (2.0 equiv.), POTASSIUM ACETATE (3.0 equiv.) in Dioxane (0.04 M) was added TRIS(DIBENZYLIDENEACETONE)DIPALLADIUM(0) (0.3 equiv.). The solution was submitted to microwave heating at 120° C. for 20 minutes. The reaction was filtered through a 1 uM HPLC frit, rinsing with additional EtOAc and the volatiles were removed in vacuo to give (2R,3R,4R,6R)-6-(3-(3-amino-6-(4,4,5,5-tetramethyl-1,3,2-dioxaborolan-2-yl)picolinamido)pyridin-4-yl)-3-ethyl-3-hydroxy-2-methyltetrahydro-2H-pyran-4-yl acetate. The crude material was taken on directly to next step.

### Synthesis of 3-amino-N-(4-((2R,4R,5S,6R)-5-ethyl-4,5-dihydroxy-6-methyltetrahydro-2H-pyran-2-yl)pyridin-3-yl)-6-(pyridazin-4-yl)picolinamide

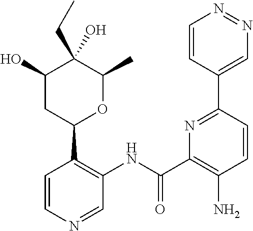 [SMILES: CC[C@]1(O)[C@H](O)C[C@H](c2ccncc2NC(=O)c2nc(-c3ccnnc3)ccc2N)O[C@@H]1C]

Method 1 was followed using 4-bromopyridazine-HBr salt (2.0 equiv.) and (2R,3R,4R,6R)-6-(3-(3-amino-6-(4,4,5,5-tetramethyl-1,3,2-dioxaborolan-2-yl)picolinamido)pyridin-4-yl)-3-ethyl-3-hydroxy-2-methyltetrahydro-2H-pyran-4-yl acetate (1.0 equiv.) to give 3-amino-N-(4-((2R,4R,5S,6R)-5-ethyl-4,5-dihydroxy-6-methyltetrahydro-2H-pyran-2-yl)pyridin-3-yl)-6-(pyridazin-4-yl)picolinamide in 47% yield. LC/MS=451.1 (M+H), Rt=0.39 min.

### Synthesis of 5-amino-N-(4-((2R,4R,5S,6R)-5-ethyl-4,5-dihydroxy-6-methyltetrahydro-2H-pyran-2-yl)pyridin-3-yl)-3′-fluoro-[2,2′-bipyridine]-6-carboxamide

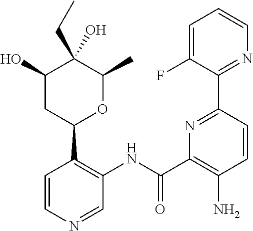 [SMILES: CC[C@]1(O)[C@H](O)C[C@H](c2ccncc2NC(=O)c2nc(-c3ncccc3F)ccc2N)O[C@@H]1C]

Method 1 was followed using 2-bromo-3-fluoropyridine (1.0 equiv.) and (2R,3R,4R,6R)-6-(3-(3-amino-6-(4,4,5,5-tetramethyl-1,3,2-dioxaborolan-2-yl)picolinamido)pyridin-4-yl)-3-ethyl-3-hydroxy-2-methyltetrahydro-2H-pyran-4-yl acetate (1.0 equiv.) to give 5-amino-N-(4-((2R,4R,5S,6R)-5-ethyl-4,5-dihydroxy-6-methyltetrahydro-2H-pyran-2-yl)pyridin-3-yl)-3′-fluoro-[2,2′-bipyridine]-6-carboxamide in 18% yield. LC/MS=468.1 (M+H), Rt=0.49 min.

### Synthesis of 5-amino-N-(4-((2R,4R,5S,6R)-5-ethyl-4,5-dihydroxy-6-methyltetrahydro-2H-pyran-2-yl)pyridin-3-yl)-3′-fluoro-2,4′-bipyridine-6-carboxamide and 3-amino-N-(4-((2R,4R,5S,6R)-5-ethyl-4,5-dihydroxy-6-methyltetrahydro-2H-pyran-2-yl)pyridin-3-yl)picolinamide

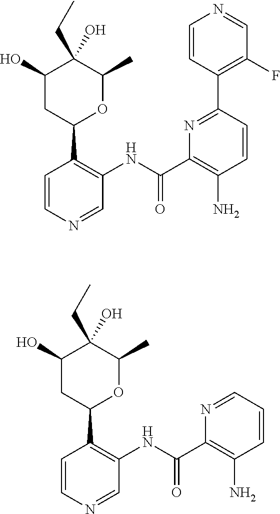 [SMILES: CC[C@]1(O)[C@H](O)C[C@H](c2ccncc2NC(=O)c2nc(-c3ccncc3F)ccc2N)O[C@@H]1C ; CC[C@]1(O)[C@H](O)C[C@H](c2ccncc2NC(=O)c2ncccc2N)O[C@@H]1C]

Method 1 was followed using (2R,3R,4R,6R)-6-(3-(3-amino-6-bromopicolinamido)pyridin-4-yl)-3-ethyl-3-hydroxy-2-methyltetrahydro-2H-pyran-4-yl acetate (1.0 equiv.) and 3-fluoro-4-(4,4,5,5-tetramethyl-1,3,2-dioxaborolan-2-yl)pyridine (1.0 equiv.) to give 5-amino-N-(4-((2R,4R,5S,6R)-5-ethyl-4,5-dihydroxy-6-methyltetrahydro-2H-pyran-2-yl)pyridin-3-yl)-3′-fluoro-2,4′-bipyridine-6-carboxamide in 38% yield, LC/MS=468.2 (M+H), Rt=0.46 min; 3-amino-N-(4-((2R,4R,5S,6R)-5-ethyl-4,5-dihydroxy-6-methyltetrahydro-2H-pyran-2-yl)pyridin-3-yl)picolinamide in 21% yield, LC/MS=373.1 (M+H), Rt=0.49 min.

### Synthesis of (+/−)-2-(3-nitropyridin-4-yl)-2H-pyran-4(3H)-one

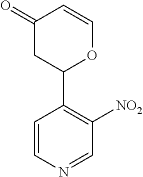 [SMILES: O=C1C=COC(c2ccncc2[N+](=O)[O-])C1]

To a solution of anhydrous zinc chloride (1.2 equiv.) in THF (0.2 M) was added 3-nitroisonicotinaldehyde (1.0 equiv.) followed by (E)-(4-methoxybuta-1,3-dien-2-yloxy)trimethylsilane (1.5 equiv.) under a nitrogen atmosphere. The reaction was allowed to stir at room temperature for 16 h, then quenched with sat. NaHCO₃. The solution was extracted with ethyl acetate, the organic phase was dried with sodium sulfate, filtered, and concentrated. The crude material was stirred in DCM and TFA (6:1, 0.2 M) for 20 min. The volatiles were removed in vacuo and the crude product was purified via silica gel column chromatography (ISCO) eluting with ethyl acetate and heptanes (0-60%). The desired fractions were concentrated to give (+/−)-2-(3-nitropyridin-4-yl)-2H-pyran-4(3H)-one as an orange solid in 76% yield. LC/MS (m/z): 221.0 (MH⁺), Rₜ=0.50 min. ¹H-NMR (300 MHz, CDCl₃): δ 9.33 (s, 1H), 8.95 (d, 1H), 7.82 (d, 1H), 7.51 (d, 1H), 6.16 (dd, 1H), 5.64 (d, 1H), 3.00 (dd, 1H), 2.70 (dd 1H).

### Synthesis of cis (+/−)-4-(4-(tert-butyldimethylsilyloxy)-3,4-dihydro-2H-pyran-2-yl)-3-nitropyridine

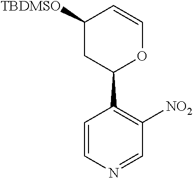 [SMILES: CC(C)(C)[Si](C)(C)O[C@H]1C=CO[C@@H](c2ccncc2[N+](=O)[O-])C1]

To a solution of (+/−)-2-(3-nitropyridin-4-yl)-2H-pyran-4(3H)-one (1.0 equiv.) in EtOH (0.1 M) was added CeCl₃-7H₂O (1.0 equiv.) and the reaction was cooled to −78° C. Sodium borohydride (1.0 equiv.) was added to the solution and the reaction was allowed to warm to room temperature. After 4 h, the reaction was quenched with water and the volatiles were removed in vacuo. The crude was partitioned between ethyl acetate and water, the organic phase was dried with brine, sodium sulfate, filtered, and concentrated. The crude material was used for the next step without further purification. LC/MS (m/z): 223.0 (MH⁺), Rₜ=0.79 min. The above material was dissolved in DCM (0.2 M) and imidazole (2.2 equiv.) and TBDMSCl (1.1 equiv.) were added. The reaction was allowed to stir overnight. Upon completion, the reaction was quenched by the addition of water, the organic phase was dried with sodium sulfate, filtered, and concentrated. The crude product was purified via silica gel column chromatography (ISCO) eluting with ethyl acetate and heptanes (0-15%) to give cis (+/−)-4-(4-(tert-butyldimethylsilyloxy)-3,4-dihydro-2H-pyran-2-yl)-3-nitropyridine as the desired product as an oil in 86% yield. LC/MS (m/z): 337.3 (MH⁺), Rₜ=1.26 min. ¹H-NMR (400 MHz, CDCl₃): δ ppm 9.25 (s, 1H), 8.83 (d, 1H), 7.75 (d, 1H), 6.49 (d, 2H), 5.71 (dd, 1H), 4.89 (dd, 1H), 4.55-4.70 (m, 1H), 2.33-2.49 (m, 1H), 1.85 (ddd, 1H), 0.84 (s, 9H), 0.07 (s, 3H), 0.05 (s, 3H).

### Synthesis of 4-((2R,4S)-4-(tert-butyldimethylsilyloxy)tetrahydro-2H-pyran-2-yl)pyridin-3-amine and 4-((2S,4R)-4-(tert-butyldimethylsilyloxy)tetrahydro-2H-pyran-2-yl)pyridin-3-amine

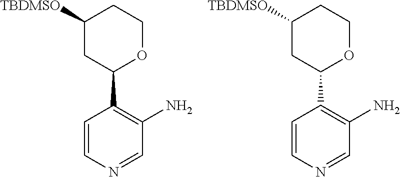 [SMILES: CC(C)(C)[Si](C)(C)O[C@H]1CCO[C@@H](c2ccncc2N)C1 ; CC(C)(C)[Si](C)(C)O[C@@H]1CCO[C@H](c2ccncc2N)C1]

To a degassed solution of cis (+/−)-4-(4-(tert-butyldimethylsilyloxy)-3,4-dihydro-2H-pyran-2-yl)-3-nitropyridine (1.0 equiv.) in EtOH (0.15 M) was added Pd/C (0.1 equiv.) and the reaction was stirred under a hydrogen balloon for 6 h. Upon completion of the reaction as monitored by LC/MS, the solution was filtered through a pad of Celite, washed with ethyl acetate and the filtrate was concentrated under vacuo to yield cis (+/−)-4-(4-(tert-butyldimethylsilyloxy)-3,4-dihydro-2H-pyran-2-yl)-3-nitropyridine in quantitative yield as a white solid, LC/MS (m/z): 309.2 (MH⁺), Rₜ=0.89 min. The enantiomers were separated via chiral HPLC (IC column, heptanes/EtOH:95/05) to yield 4-((2R,4S)-4-(tert-butyldimethylsilyloxy)tetrahydro-2H-pyran-2-yl)pyridin-3-amine (99% ee) and 4-((2S,4R)-4-(tert-butyldimethylsilyloxy)tetrahydro-2H-pyran-2-yl)pyridin-3-amine (99% ee).

### Synthesis of (+/−)-3-hydroxy-2-methyl-6-(3-nitropyridin-4-yl)dihydro-2H-pyran-4(3H)-one

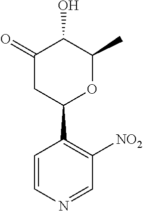 [SMILES: C[C@H]1O[C@@H](c2ccncc2[N+](=O)[O-])CC(=O)[C@@H]1O]

To a solution of cis (+/−)-4-(6-methyl-4-(triethylsilyloxy)-3,6-dihydro-2H-pyran-2-yl)-3-nitropyridine (1.0 equiv.) in DCM (0.3 M) at 0° C. was added a solution of freshly distilled DMDO in acetone (1.0 equiv.). The reaction was monitored by TLC and after 2 h, another 1.0 equiv. of DMDO was added. After 2 h at room temperature, the reaction was complete as indicated by LC/MS. The volatiles were removed under vacuo and the crude material was dissolved in THF and 1N HCl (5:4) was added. The solution was stirred for 30 min, then neutralized with 1N NaOH. Ethyl acetate was added, the organic phase was dried with sodium sulfate, filtered, and concentrated. The crude material was purified via silica gel column chromatography eluting with ethyl acetate and heptanes (0-50%) to give (+/−)-3-hydroxy-2-methyl-6-(3-nitropyridin-4-yl)dihydro-2H-pyran-4(3H)-one in 35% yield as a white solid. LC/MS (m/z): 253.0 (MH⁺), Rₜ=0.50 min. ¹H-NMR (400 MHz, CDCl₃): δ ppm 9.24 (s, 1H), 8.90 (d, 1H), 7.88 (d, 1H), 5.36 (dd, 1H), 3.96 (ddd, 1H), 3.63 (m 1H), 3.58 (d, 1H), 3.15 (dd, 1H), 2.60 (m, 1H), 1.56 (d, J=4 Hz, 3H).

### Synthesis of (+/−)-3-(tert-butyldimethylsilyloxy)-2-methyl-6-(3-nitropyridin-4-yl)dihydro-2H-pyran-4(3H)-one

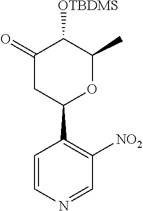 [SMILES: C[C@H]1O[C@@H](c2ccncc2[N+](=O)[O-])CC(=O)[C@@H]1O[Si](C)(C)C(C)(C)C]

To a solution of (+/−)-3-hydroxy-2-methyl-6-(3-nitropyridin-4-yl)dihydro-2H-pyran-4(3H)-one (1.0 equiv.) in DCM (0.2 M) was added imidazole (2.4 equiv.) followed by TBDMSCl (1.2 equiv.). The reaction was stirred at room temperature until completion (overnight), then partitioned between water and ethyl acetate. The organic phase was dried with sodium sulfate, filtered and concentrated. The crude material was purified via silica gel column chromatography eluting with ethyl acetate and heptanes (0-50%) to give (+/−)-3-(tert-butyldimethylsilyloxy)-2-methyl-6-(3-nitropyridin-4-yl)dihydro-2H-pyran-4(3H)-one as a white solid in 66% yield. LC/MS (m/z): 367.1 (MH⁺), Rₜ=1.21 min. ¹H-NMR (400 MHz, CDCl₃): δ ppm 9.22 (s, 1H), 8.87 (d, 1H), 7.84 (d, 1H), 5.35 (dd, 1H), 3.95 (d, 1H), 3.77 (dd, 1H), 3.01 (dd, 1H), 2.51 (m, 1H), 1.48 (d, 3H), 0.92 (s, 9H), 0.19 (s, 3H), 0.06 (s, 3H).

### Synthesis of (+/−)-3-(tert-butyldimethylsilyloxy)-2-methyl-6-(3-nitropyridin-4-yl)tetrahydro-2H-pyran-4-ol

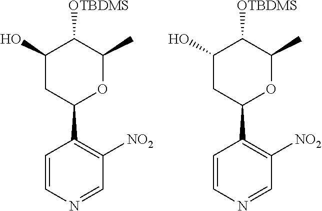 [SMILES: C[C@H]1O[C@@H](c2ccncc2[N+](=O)[O-])C[C@@H](O)[C@@H]1O[Si](C)(C)C(C)(C)C ; C[C@H]1O[C@@H](c2ccncc2[N+](=O)[O-])C[C@H](O)[C@@H]1O[Si](C)(C)C(C)(C)C]

To a solution of (+/−)-3-(tert-butyldimethylsilyloxy)-2-methyl-6-(3-nitropyridin-4-yl)dihydro-2H-pyran-4(3H)-one (1.0 equiv.) in MeOH (0.2 M) at 0° C. was added solid sodium borohydride (1.0 equiv.) in one portion and the reaction was stirred for 10 min. Added sat. NH₄Cl and concentrated the volatiles in vacuo. To the aqueous was added ethyl acetate, the organic phase was dried with sodium sulfate, filtered, and concentrated to yield an orange oil. The crude was purified via silica gel column chromatography eluting with ethyl acetate and heptanes (0-25%) to afford (+/−)-3-(tert-butyldimethylsilyloxy)-2-methyl-6-(3-nitropyridin-4-yl)tetrahydro-2H-pyran-4-ol as a mixture of two separable diastereomers in 2:1 ratio. Diastereomer A: LC/MS (m/z): 369.2 (MH⁺), Rₜ=1.18 min. Diastereomer B: LC/MS (m/z): 369.2 (MH⁺), Rₜ=1.19 min.

### Synthesis of (+/−)-3-(tert-butyldimethylsilyloxy)-2-methyl-6-(3-nitropyridin-4-yl)tetrahydro-2H-pyran-4-yl acetate

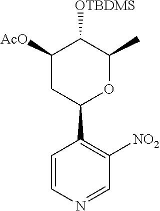 [SMILES: CC(=O)O[C@@H]1C[C@H](c2ccncc2[N+](=O)[O-])O[C@H](C)[C@H]1O[Si](C)(C)C(C)(C)C]

To a solution of (+/−)-3-(tert-butyldimethylsilyloxy)-2-methyl-6-(3-nitropyridin-4-yl)tetrahydro-2H-pyran-4-ol (1.0 equiv.) in pyridine (0.4 M) was added Ac₂O (14 equiv.). The reaction was stirred at room temperature overnight. Upon completion, water was added, the volatiles were removed in vacuo, the crude was partitioned between ethyl acetate and water, the organic phase was dried with sodium sulfate, filtered, and concentrated. The crude material was purified via silica gel column chromatography eluting with heptanes and ethyl acetate (0-20%) to afford (+/−)-3-(tert-butyldimethylsilyloxy)-2-methyl-6-(3-nitropyridin-4-yl)tetrahydro-2H-pyran-4-yl acetate in 75% yield as a clear oil. LC/MS (m/z): 411.2 (MH⁺), Rₜ=1.29 min. ¹H-NMR (400 MHz, CDCl₃): δ ppm 9.03 (s, 1H), 8.68 (d, 1H), 7.59 (d, 1H), 5.07 (dd, 1H), 4.87 (ddd, 1H), 3.38-3.47 (m, 1H), 3.33 (t, 1 Hz), 2.50 (ddd, 1H), 1.95 (s, 3H), 1.32-1.47 (m, 1H), 1.24 (d, 3H), 0.77-0.81 (m, 9H), 0.03 (s, 3H), 0.02 (s, 3H).

### Synthesis of (+/−)-6-(3-aminopyridin-4-yl)-3-(tert-butyldimethylsilyloxy)-2-methyltetrahydro-2H-pyran-4-yl acetate

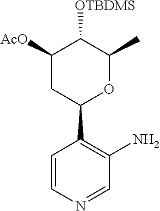 [SMILES: CC(=O)O[C@@H]1C[C@H](c2ccncc2N)O[C@H](C)[C@H]1O[Si](C)(C)C(C)(C)C]

To a degassed solution of (+/−)-3-(tert-butyldimethylsilyloxy)-2-methyl-6-(3-nitropyridin-4-yl)tetrahydro-2H-pyran-4-yl acetate (1.0 equiv.) in EtOH and EtOAc (1:1, 0.09 M) was added Pd/C (0.1 equiv.) and the reaction was stirred under a hydrogen balloon for 4 hrs. The solution was filtered through a pad of Celite, the Celite was washed with ethyl acetate and the filtrate was concentrated under vacuo to afford (+/−)-6-(3-aminopyridin-4-yl)-3-(tert-butyldimethylsilyloxy)-2-methyltetrahydro-2H-pyran-4-yl acetate as a clear oil in 95% yield. LC/MS (m/z): 381.1 (MH⁺), Rₜ=0.98 min. The material was separated via chiral HPLC (IC column, heptane:IPA 95:05) to give (2R,3R,4R,6S)-6-(3-aminopyridin-4-yl)-3-(tert-butyldimethylsilyloxy)-2-methyltetrahydro-2H-pyran-4-yl acetate (>99% ee) and (2S,3S,4S,6R)-6-(3-aminopyridin-4-yl)-3-(tert-butyldimethylsilyloxy)-2-methyltetrahydro-2H-pyran-4-yl acetate (>99% ee).

### Synthesis of (+/−)-3-(tert-butyldimethylsilyloxy)-2-methyl-6-(3-nitropyridin-4-yl)tetrahydro-2H-pyran-4-yl acetate

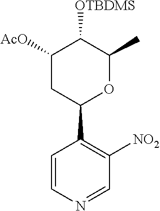 [SMILES: CC(=O)O[C@H]1C[C@H](c2ccncc2[N+](=O)[O-])O[C@H](C)[C@H]1O[Si](C)(C)C(C)(C)C]

To a solution of (+/−)-3-(tert-butyldimethylsilyloxy)-2-methyl-6-(3-nitropyridin-4-yl)tetrahydro-2H-pyran-4-ol (1.0 equiv.) in pyridine (0.2M) was added Ac₂O (20 equiv.). The reaction was stirred at room temperature overnight. Upon completion, the volatiles were removed in vacuo, the crude was partitioned between ethyl acetate and water, the organic phase was dried with sodium sulfate, filtered, and concentrated. The crude material was purified via silica gel column chromatography eluting with heptanes and ethyl acetate (0-30%) to afford (+/−)-3-(tert-butyldimethylsilyloxy)-2-methyl-6-(3-nitropyridin-4-yl)tetrahydro-2H-pyran-4-yl acetate in 57% yield as a clear oil. LC/MS (m/z): 381.1 (MH⁺), Rₜ=0.98 min.

### Synthesis of (+/−)-6-(3-aminopyridin-4-yl)-3-(tert-butyldimethylsilyloxy)-2-methyltetrahydro-2H-pyran-4-yl acetate

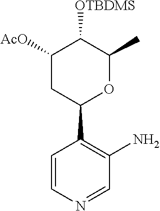 [SMILES: CC(=O)O[C@H]1C[C@H](c2ccncc2N)O[C@H](C)[C@H]1O[Si](C)(C)C(C)(C)C]

To a degassed solution of (+/−)-3-(tert-butyldimethylsilyloxy)-2-methyl-6-(3-nitropyridin-4-yl)tetrahydro-2H-pyran-4-yl acetate (1.0 equiv.) in EtOH (0.06 M) was added Pd/C (0.1 equiv.) and the reaction was stirred under a hydrogen balloon for 15 hrs. The solution was filtered through a pad of Celite, the Celite was washed with ethyl acetate and the filtrate was concentrated in vacuo to afford (+/−)-6-(3-aminopyridin-4-yl)-3-(tert-butyldimethylsilyloxy)-2-methyltetrahydro-2H-pyran-4-yl acetate as a clear oil in 90% yield. LC/MS (m/z): 411.2 (MH⁺), Rₜ=1.30 min. The material was separated via chiral HPLC (OD-H column, heptane:EtOH 98:02) to give (2S,3S,4R,6R)-6-(3-aminopyridin-4-O-3-(tert-butyldimethylsilyloxy)-2-methyltetrahydro-2H-pyran-4-yl acetate (>99% ee) and (2R,3R,4S,6S)-6-(3-aminopyridin-4-yl)-3-(tert-butyldimethylsilyloxy)-2-methyltetrahydro-2H-pyran-4-yl acetate (>99% ee).

### Synthesis of 2,2,2-trifluoro-1-(3-nitropyridin-4-yl)ethanone

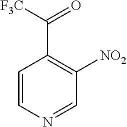 [SMILES: O=C(c1ccncc1[N+](=O)[O-])C(F)(F)F]

To a solution of 3-nitroisonicotinaldehyde (1.0 equiv.) in DME (0.3 M) was added CsF (0.1 equiv.) and the solution was cooled to 0° C. Trimethyl(trifluoromethyl)silane (1.1 equiv.) was added dropwise and the reaction was allowed to warm to room temperature. After 5 h, 1N HCl was added and the reaction was stirred overnight at room temperature. The solution was partitioned between ethyl acetate and water, the organic phase was dried with sodium sulfate, filtered and concentrated. The crude material was purified via silica gel column chromatography eluting with ethyl acetate and heptanes (0-30%). The pure fractions were concentrated to give an oil that solidified upon standing. This oil was dissolved in DCM (0.2 M) and cooled to 0° C. Dess-Martin Periodinane (1.5 equiv.) was added to the reaction and allowed to warm to room temperature. After 3 h, the reaction was washed with sat. NaHCO₃, the organic phase was dried with sodium sulfate, filtered and concentrated under vacuo. The crude material was purified via silica gel column chromatography eluting with ethyl acetate and heptanes (0-50%) to yield 2,2,2-trifluoro-1-(3-nitropyridin-4-yl) ethanone in 81% yield as a white solid. LC/MS (m/z): 239.0 (M+H₂O⁺), Rₜ=0.52 min.

### Synthesis of 2-(3-nitropyridin-4-yl)-2-(trifluoromethyl)-2H-pyran-4(3H)-one

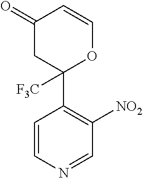 [SMILES: O=C1C=COC(c2ccncc2[N+](=O)[O-])(C(F)(F)F)C1]

To a solution of anhydrous zinc chloride (1.5 equiv.) in THF (0.2 M) was added 2,2,2-trifluoro-1-(3-nitropyridin-4-yl)ethanone (1.0 equiv.) under an atmosphere of nitrogen. Danishefsky's diene (1.5 equiv.) was added to the solution and the reaction was stirred at room temperature for 3 days. Upon consumption of the starting material, the reaction was quenched by the addition of saturated NaHCO₃ and extracted with ethyl acetate. The organic phase was dried with sodium sulfate, filtered and concentrated to give the aldol adduct. The crude material was dissolved in DCM and TFA (5:1) and stirred at room temperature for 3 h. The solution was concentrated and purified via silica gel column chromatography eluting with ethyl acetate and heptanes 0-20% then 50%). The pure fractions were concentrated to give 2-(3-nitropyridin-4-yl)-2-(trifluoromethyl)-2H-pyran-4(3H)-one in 73% yield. LC/MS (m/z): 330.1 (MH⁺), Rₜ=0.70 min.

### Synthesis of (+/−)-2-(3-nitropyridin-4-yl)-2-(trifluoromethyl)-3,4-dihydro-2H-pyran-4-yl acetate

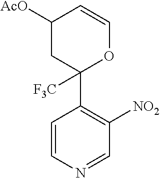 [SMILES: CC(=O)OC1C=COC(c2ccncc2[N+](=O)[O-])(C(F)(F)F)C1]

To a solution of 2-(3-nitropyridin-4-yl)-2-(trifluoromethyl)-2H-pyran-4(3H)-one (1.0 equiv.) in EtOH (0.2 M) was added CeCl₃-7H₂O (1.0 equiv.) and the reaction was cooled to 0° C. Sodium borohydride (1.0 equiv.) was added and the reaction was stirred for 30 min at 0° C. Water was added followed by ethyl acetate. The volatiles were removed under vacuo and the crude was partitioned between ethyl acetate and water. The organic phase was dried with sodium sulfate, filtered and concentrated. The crude material was used for the next step without further purification. LC/MS (m/z): 291 (MH⁺), Rₜ=0.66 min. To a solution of the above material in pyridine was added acetic anhydride (1:1, 0.2 M) and the solution was stirred at room temperature for 2 hours. Upon completion of the reaction, the solution was concentrated under vacuo, then diluted with ethyl acetate and water. The organic phase was dried with sodium sulfate, filtered and concentrated. The crude material was purified via silica gel column chromatography eluting with ethyl acetate and heptanes (0-50%) to give (+/−)-2-(3-nitropyridin-4-yl)-2-(trifluoromethyl)-3,4-dihydro-2H-pyran-4-yl acetate as the desired product as a clear oil in 62% yield. LC/MS (m/z): 333.0 (MH⁺), Rₜ=0.85 min. ¹H-NMR (300 MHz, CDCl₃): δ ppm 8.84 (d, 1H), 8.71 (s, 1H), 7.56 (d, 1H), 6.37 (d, 1H), 4.90-5.06 (m, 2H), 2.97-3.17 (m, 1H), 2.38 (dd, 1H), 2.09 (s, 3H).

### Synthesis of (+/−)-2-(3-aminopyridin-4-yl)-2-(trifluoromethyl)-3,4-dihydro-2H-pyran-4-yl acetate

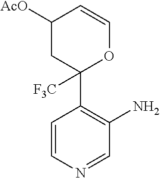 [SMILES: CC(=O)OC1C=COC(c2ccncc2N)(C(F)(F)F)C1]

To a solution of (+/−)-2-(3-nitropyridin-4-yl)-2-(trifluoromethyl)-3,4-dihydro-2H-pyran-4-yl acetate (1.0 equiv.) in AcOH (0.08 M) was added iron powder (10 equiv.) and the reaction was stirred for 2 h. The solution was diluted with methanol and filtered through a pad of Celite and washed with methanol. The filtrate was concentrated under vacuo and partitioned between ethyl acetate and sat. NaHCO₃. The organic phase was dried with sodium sulfate, filtered, and concentrated. The crude material was used for the next step without further purification. LC/MS (m/z): 303.1 (MH⁺), Rₜ=0.54 min.

### Synthesis of (+/−)-2-(3-aminopyridin-4-yl)-2-(trifluoromethyl)tetrahydro-2H-pyran-4-yl acetate and (+/−)-4-(2-(trifluoromethyl)tetrahydro-2H-pyran-2-yl)pyridin-3-amine

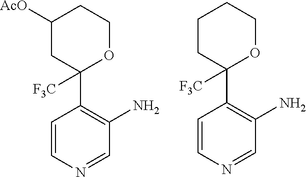 [SMILES: CC(=O)OC1CCOC(c2ccncc2N)(C(F)(F)F)C1 ; Nc1cnccc1C1(C(F)(F)F)CCCCO1]

To a degassed solution of (+/−)-2-(3-nitropyridin-4-yl)-2-(trifluoromethyl)-3,4-dihydro-2H-pyran-4-yl acetate (1.0 equiv.) in EtOH (0.18 M) was added Pd/C (0.1 equiv.) and the solution was stirred under a hydrogen balloon. After 4 h, the solution was filtered through a pad of Celite and washed with ethyl acetate. The filtrate was concentrated under vacuo to give the product as a mixture of two compounds in a 2:1 ratio. LC/MS (m/z): 247.1 (MH⁺), Rₜ=0.51 min and LC/MS (m/z): 305.0 (MH⁺), Rₜ=0.55 min.

### Synthesis of ((2R,3R,4R)-2-((triisopropylsilyloxy)methyl)-3,4-dihydro-2H-pyran-3,4-diyl)bis(oxy)bis(triisopropylsilane)

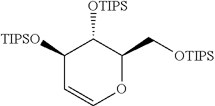

To a solution of D-Glucal (1.0 equiv.) in DCM (1M) was added 2,6-lutidine (6.6 equiv.) and the reaction was cooled to 0° C. under an atmosphere of nitrogen. TipsOTf (4.5 equiv.) was added dropwise via an addition funnel and upon completion, the solution was allowed to warm to room temperature and stirred overnight. TLC of the solution (10:1 heptanes and ethyl acetate) indicated one major non-polar spot. The reaction was partitioned between DCM and water, the organic phase was washed with water (3 times), then dried with sodium sulfate and concentrated. The crude material was purified by filtering through a plug of silica gel eluting with 100% heptanes then 1:2 DCM and heptanes. The solution was concentrated in vacuo to give ((2R,3R,4R)-2-((triisopropylsilyloxy)methyl)-3,4-dihydro-2H-pyran-3,4-diyl)bis(oxy)bis(triisopropylsilane) as a yellow oil in 97% yield. ¹H-NMR (400 MHz, CDCl₃): δ ppm 6.36 (d, 1H), 4.79-4.82 (m, 1H), 4.22-4.24 (m, 2H), 4.04-4.06 (m, 2H), 3.82 (dd, 1H), 1.07 (s, 63H).

### Synthesis of 4-((2R,3R,4R)-3,4-bis(triisopropylsilyloxy)-2-((triisopropylsilyloxy)methyl)-3,4-dihydro-2H-pyran-6-yl)-3-nitropyridine

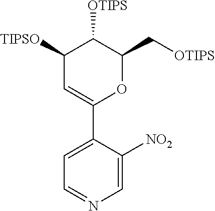

To a solution of ((2R,3R,4R)-2-((triisopropylsilyloxy)methyl)-3,4-dihydro-2H-pyran-3,4-diyl)bis(oxy)bis(triisopropylsilane) (1.0 equiv.) in anhydrous THF (0.2 M) at −78° C. under a nitrogen atmosphere was added t-BuLi (1.7 M solution in pentane, 4 equiv.) dropwise via an addition funnel. The light brown solution was stirred at −78° C. for 30 min, then allowed to warm to 0° C. and stirred at that temperature for 1 h. Trimethyl borate (10 equiv.) was added in one portion at 0° C., stirred at that temperature for 30 min, then allowed to warm to room temperature and stirred overnight. The solution was quenched by the addition of water, partitioned with ethyl acetate, the organic phase was dried with sodium sulfate, filtered and concentrated. The crude material was used for the next step without further purification. To a degassed solution of the above crude (1.0 equiv.) in DME and 2 M Na₂CO₃ (2:1, 0.2M) was added 4-chloro-3-nitropyridine (1.5 equiv.) followed by bis(triphenylphosphine)palladium(II)chloride (0.1 equiv.). The reaction was heated to 80° C. for 3 h. Upon cooling to room temperature, the solution was diluted with ethyl acetate and water. The aqueous phase was extracted 3 times with ethyl acetate, the organics were combined, dried with sodium sulfate, filtered and concentrated. The crude material was purified via silica gel column chromatography eluting with ethyl acetate and heptanes (0-10%). The pure fractions were combined and concentrated to yield 4-((2R,3R,4R)-3,4-bis(triisopropylsilyloxy)-2-((triisopropylsilyloxy)methyl)-3,4-dihydro-2H-pyran-6-yl)-3-nitropyridine in 85% yield as a dark orange oil ¹H-NMR (400 MHz, CDCl₃): δ ppm 8.93 (s, 1H), 8.73 (d, 1H), 7.44 (d, 1H), 5.29 (dd, 1H), 4.38 (t, 1H), 4.19 (m, 1H), 4.02 (d, 1H), 1.07 (m, 63H).

### Synthesis of 4-((2R,3R,4R)-3,4-bis(triisopropylsilyloxy)-2-(triisopropylsilyloxy)methyl)-3,4-dihydro-2H-pyran-6-yl)pyridin-3-amine

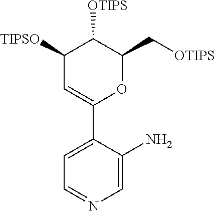

To a solution of 4-((2R,3R,4R)-3,4-bis(triisopropylsilyloxy)-2-((triisopropylsilyloxy)methyl)-3,4-dihydro-2H-pyran-6-yl)-3-nitropyridine (1.0 equiv.) in AcOH (0.1 M) was added iron powder (5 equiv.) and the reaction was stirred at room temperature for 2 hours. Upon completion, the solution was filtered through a pad of Celite and washed with methanol. The filtrate was concentrated, then the crude material was dissolved in ethyl acetate and the organic phase was washed with sat. NaHCO₃. The organic was dried with sodium sulfate, filtered and concentrated to give 4-((2R,3R,4R)-3,4-bis(triisopropylsilyloxy)-2-((triisopropylsilyloxy)methyl)-3,4-dihydro-2H-pyran-6-yl)pyridin-3-amine as the desired product in 83% yield as an oil. LC/MS (m/z): 707.7 (MH⁺), Rₜ=0.55 min (95/95 method on UPLC).

### Synthesis of 4-((2R,4R,5R,6R)-4,5-bis(triisopropylsilyloxy)-6-((triisopropylsilyloxy)methyl)tetrahydro-2H-pyran-2-yl)pyridin-3-amine

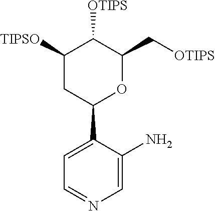

To a degassed solution of 4-((2R,3R,4R)-3,4-bis(triisopropylsilyloxy)-2-((triisopropylsilyloxy)methyl)-3,4-dihydro-2H-pyran-6-yl)-3-nitropyridine (1.0 equiv.) in EtOH (0.1 M) was added Pd(OH)₂ (0.2 equiv.) and the reaction was stirred at room temperature under a hydrogen balloon for 2 days. Filtered through a pad of Celite and washed with methanol. The filtrate was concentrated in vacuo to give 4-((2R,4R,5R,6R)-4,5-bis(triisopropylsilyloxy)-6-((triisopropylsilyloxy)methyl)tetrahydro-2H-pyran-2-yl)pyridin-3-amine as an oil in quantitative yield. LC/MS (m/z): 709.8 (MH⁺), Rₜ=0.58 min (95/95 method on UPLC).

### Synthesis of (2S,3R,4R)-6-(3-nitropyridin-4-yl)-3,4-bis(triisopropylsilyloxy)-3,4-dihydro-2H-pyran-2-carbaldehyde

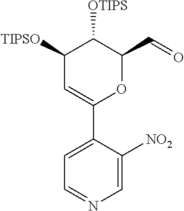

To a solution of ((2R,3R,4R)-6-(3-nitropyridin-4-yl)-3,4-bis(triisopropylsilyloxy)-3,4-dihydro-2H-pyran-2-yl)methanol (1.0 equiv.) in DCM (0.2 M) at 0° C. was added Dess-Martin Periodinane (1.5 equiv.) and the reaction was allowed to warm to room temperature over time. After 2 h, the reaction was completed by TLC. The solution was quenched by the addition of water, the organic phase was dried with sodium sulfate, filtered and concentrated. The crude material was purified via silica gel column chromatography eluting with ethyl acetate and heptanes (0-20%). The pure fractions were concentrated to yield (2S,3R,4R)-6-(3-nitropyridin-4-yl)-3,4-bis(triisopropylsilyloxy)-3,4-dihydro-2H-pyran-2-carbaldehyde as an yellow oil in 52% yield. ¹H-NMR (400 MHz, CDCl₃): δ ppm 9.66 (d, 1H), 9.02 (s, 1H), 8.81 (d, 1H), 7.48 (d, 1H), 5.43-5.58 (m, 1H), 4.52-4.61 (m, 1H), 4.30-4.44 (m, 1H), 4.05-4.25 (m, 1H), 1.03-1.25 (m, 42H).

### Synthesis of 4-((2R,3R,4R)-3,4-bis(triisopropylsilyloxy)-2-vinyl-3,4-dihyrdo-2H-pyran-6-yl-3-nitropyridine

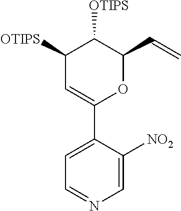

To a solution of methyltriphenylphosphonium bromide (1.5 equiv) in THF (0.20 M) was added slowly lithium bis(trimethylsilyl)amide (1.45 equiv.) at −78° C. The cooling bath was removed and the glide solution was stirred for 1 hr allowing the reaction to warm to room temperature. The reaction was again cooled to −78° C. and (2S,3R,4R)-6-(3-nitropyridin-4-yl)-3,4-bis(triisopropylsilyloxy)-3,4-dihyrdo-2H-pyran-2-carbaldehyde (1 equiv.) in THF (1 mL) was added to the ylide solution maintaining an internal temperature of >/=−60° C. After addition, the cooling bath was removed and the reaction was allowed to stir for 2.5 hrs. To the reaction was added NH₄Cl_{(sat.)} (10 mL) and ethyl acetate (25 mL). Upon separation, the organic layer was washed further with NH₄Cl_{(sat.)} (3×10 mL), with NaCl_{(sat.)} (15 mL), dried over MgSO₄, filtered, and the volatiles were removed in vacuo. Purification was completed by silica gel column chromatography via ISCO (24 g column, 0-25% EtOAc:Hexanes, 15 min run time, 35 mL/min) to yield 4-((2R,3R,4R)-3,4-bis(triisopropylsilyloxy)-2-vinyl-3,4-dihyro-2H-pyran-6-yl-3-nitropyridine as the desired product in 65% yield. ¹H-NMR (400 MHz, CDCl₃): δ ppm 8.94 (s, 1H), 8.75 (d, 1H), 7.44 (d, 1H), 6.21-6.43 (m, 1H), 5.36 (dd, 1H), 5.11-5.27 (m, 2H), 4.68 (d, 1H), 4.22 (dd, 1H), 3.99-4.10 (m, 1H), 0.93-1.29 (m, 42H).

### Synthesis of 4-((2R,4R,5R,6R)-6-ethyl-4,5-bis(triisopropylsilyloxy)tetrahydro-2H-pyran-2-yl)pyridin-3-amine

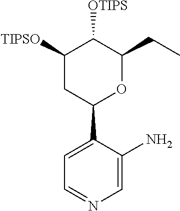

To a degassed solution of 4-((2R,3R,4R)-3,4-bis(triisopropylsilyloxy)-2-vinyl-3,4-dihydro-2H-pyran-6-yl)-3-nitropyridine (1.0 equiv.) in EtOH (0.03 M) was added Pd(OH)₂ (0.2 equiv.) and the reaction was stirred under a hydrogen balloon for 30 hours. Upon completion of the reaction, the solution was filtered through a pad of Celite and concentrated under vacuo to give 4-((2R,4R,5R,6R)-6-ethyl-4,5-bis(triisopropylsilyloxy)tetrahydro-2H-pyran-2-yl)pyridin-3-amine as an oil in 95% yield. LC/MS (m/z): 551.6 (MH⁺) Rₜ=1.25 min.

### Synthesis of (2R,3S,4R)-2-(hydroxymethyl)-6-(3-nitropyridin-4-yl)-3,4-dihydro-2H-pyran-3,4-diol

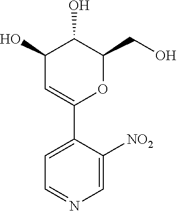 [SMILES: O=[N+]([O-])c1cnccc1C1=C[C@@H](O)[C@H](O)[C@@H](CO)O1]

To a solution of 4-((2R,3R,4R)-3,4-bis(triisopropylsilyloxy)-2-((triisopropylsilyloxy)methyl)-3,4-dihydro-2H-pyran-6-yl)-3-nitropyridine (1.0 equiv.) in THF (0.3 M) was added TBAF (3.3 equiv.). The solution was stirred at room temperature for 2 days. The reaction was concentrated under vacuo and purified via silica gel column chromatography eluting with dichloromethane and methanol (10% MeOH). The compound was redissolved in THF and MeOH (5:3) followed by the addition of DOWEX and CaCO₃ in order to remove excess TBAF. Upon stirring for 1 h at room temperature, the solution was filtered through Celite and washed with MeOH. The filtrate was concentrated under vacuo to afford (2R,3S,4R)-2-(hydroxymethyl)-6-(3-nitropyridin-4-yl)-3,4-dihydro-2H-pyran-3,4-diol as an off-white solid in 52% yield. LC/MS (m/z): 269.1 (MH⁺), Rₜ=0.34 min.

### Synthesis of (2R,3S,4R)-6-(3-nitropyridin-4-yl)-2-(trityloxymethyl)-3,4-dihydro-2H-pyran-3,4-diol

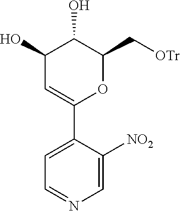

To a solution of (2R,3S,4R)-2-(hydroxymethyl)-6-(3-nitropyridin-4-yl)-3,4-dihydro-2H-pyran-3,4-diol (1.0 equiv.) in pyridine (0.37 M) was added trityl chloride (1.2 equiv.) and the reaction was stirred at room temperature for 3 days. Upon completion, the solution was concentrated under vacuo and purified via silica gel column chromatography eluting with ethyl acetate and heptanes (0-100% ethyl acetate). The pure fractions were concentrated to yield (2R,3S,4R)-6-(3-nitropyridin-4-yl)-2-(trityloxymethyl)-3,4-dihydro-2H-pyran-3,4-diol in 68% yield as an off white foam. LC/MS (m/z): 511.4 (MH⁺), Rₜ=1.01 min.

### Synthesis of (2R,3S,4R)-6-(3-nitropyridin-4-yl)-2-(trityloxymethyl)-3,4-dihydro-2H-pyran-3,4-diyl diacetate

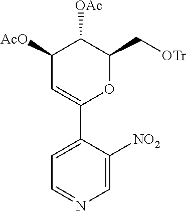

To a solution of (2R,3S,4R)-6-(3-nitropyridin-4-yl)-2-(trityloxymethyl)-3,4-dihydro-2H-pyran-3,4-diol (1.0 equiv.) in pyridine was added Ac₂O (3.0 equiv.) and the reaction was stirred at room temperature overnight. Upon completion of the reaction, the solution was concentrated to dryness under vacuo and partitioned between ethyl acetate and water. The organic phase was dried with sodium sulfate, filtered, and concentrated. The product (2R,3S,4R)-6-(3-nitropyridin-4-yl)-2-(trityloxymethyl)-3,4-dihydro-2H-pyran-3,4-diyl diacetate was used for the next step without further purification. LC/MS (m/z): 595.5 (MH⁺), Rₜ=1.21 min.

### Synthesis of (2R,3S,4R)-2-(hydroxymethyl)-6-(3-nitropyridin-4-yl)-3,4-dihydro-2H-pyran-3,4-diyl diacetate

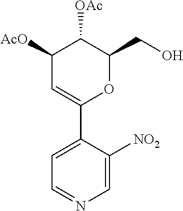 [SMILES: CC(=O)O[C@H]1[C@H](OC(C)=O)C=C(c2ccncc2[N+](=O)[O-])O[C@@H]1CO]

To a solution of (2R,3S,4R)-6-(3-nitropyridin-4-yl)-2-(trityloxymethyl)-3,4-dihydro-2H-pyran-3,4-diyl diacetate (1.0 equiv.) in DCM (0.6M) was added iron(III) chloride (3.0 equiv.) and the reaction was stirred at room temperature for 12 h. Upon completion, the reaction was quenched by the addition of water and extracted with DCM. The organic phase was dried with sodium sulfate, filtered and concentrated. The crude material was purified via silica gel column chromatography eluting with ethyl acetate and heptanes (0-50%) to give (2R,3S,4R)-2-(hydroxymethyl)-6-(3-nitropyridin-4-yl)-3,4-dihydro-2H-pyran-3,4-diyl diacetate as a clear oil in 47% yield. LC/MS (m/z): 353.1 (MH⁺), Rₜ=0.63 min.

### Synthesis of (2R,3S,4R)-6-(3-nitropyridin-4-yl)-2-(tosyloxymethyl)-3,4-dihydro-2H-pyran-3,4-diyl diacetate

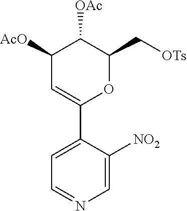 [SMILES: CC(=O)O[C@H]1[C@H](OC(C)=O)C=C(c2ccncc2[N+](=O)[O-])O[C@@H]1COS(=O)(=O)c1ccc(C)cc1]

To a solution of (2R,3S,4R)-2-(hydroxymethyl)-6-(3-nitropyridin-4-yl)-3,4-dihydro-2H-pyran-3,4-diyl diacetate (1.0 equiv.) in pyridine (0.2 M) at 0° C. was added TsCl (1.1 equiv.) and the reaction was allowed to warm to room temperature and stirred for 6 h. Another 0.5 equiv. of TsCl was added to the reaction and the solution was stirred overnight. Upon completion, the solution was concentrated under vacuo and partitioned between ethyl acetate and water. The organic phase was dried with sodium sulfate, filtered and concentrated. The crude material was purified via silica gel column chromatography eluting with ethyl acetate and heptanes (0-30% to 50%) to give (2R,3S,4R)-6-(3-nitropyridin-4-yl)-2-(tosyloxymethyl)-3,4-dihydro-2H-pyran-3,4-diyl diacetate as a clear oil in 73% yield. LC/MS (m/z): 507.2 (MH⁺), Rₜ=0.92 min.

### Synthesis of (2R,3S,6S)-6-(3-aminopyridin-4-yl)-2-(tosyloxymethyl)tetrahydro-2H-pyran-3-yl acetate and (2R,3S,4R,6S)-6-(3-aminopyridin-4-yl)-2-(tosyloxymethyl)tetrahydro-2H-pyran-3,4-diyl diacetate

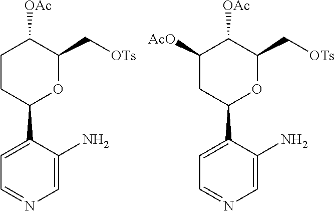 [SMILES: CC(=O)O[C@H]1CC[C@H](c2ccncc2N)O[C@@H]1COS(=O)(=O)c1ccc(C)cc1 ; CC(=O)O[C@H]1[C@H](OC(C)=O)C[C@H](c2ccncc2N)O[C@@H]1COS(=O)(=O)c1ccc(C)cc1]

To a degassed solution of (2R,3S,4R)-6-(3-nitropyridin-4-yl)-2-(tosyloxymethyl)-3,4-dihydro-2H-pyran-3,4-diyl diacetate (1.0 equiv.) in EtOH and ethyl acetate (1:1, 0.04M) was added Pd/C (0.1 equiv.) and the reaction was stirred under a hydrogen balloon for 12 h. A mixture of the two products shown above was identified by LC/MS. The reaction was filtered through a pad of Celite and washed with ethyl acetate. The filtrate was concentrated to give (2R,3S,6S)-6-(3-aminopyridin-4-yl)-2-(tosyloxymethyl)tetrahydro-2H-pyran-3-yl acetate and (2R,3S,4R,6S)-6-(3-aminopyridin-4-yl)-2-(tosyloxymethyl)tetrahydro-2H-pyran-3,4-diyl diacetate as a mixture of two products in 95% yield. LC/MS (m/z): 479.2 (MH⁺), Rₜ=0.69 min and 421.2 (MH⁺), Rₜ=0.67 min.

### Synthesis of (2R,3S,4R,6R)-6-(3-(6-(2,6-difluorophenyl)-5-fluoropicolinamido)pyridin-4-yl)-2-(tosyloxymethyl)tetrahydro-2H-pyran-3,4-diyl diacetate and (2R,3S,6R)-6-(3-(6-(2,6-difluorophenyl)-5-fluoropicolinamido)pyridin-4-yl)-2-(tosyloxymethyl)tetrahydro-2H-pyran-3-yl acetate

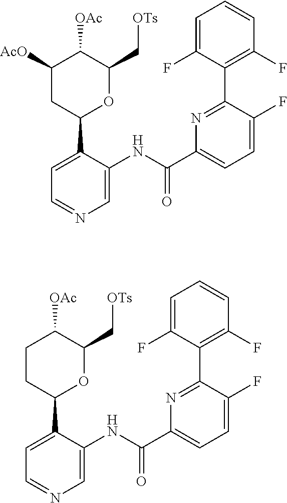 [SMILES: CC(=O)O[C@H]1[C@H](OC(C)=O)C[C@H](c2ccncc2NC(=O)c2ccc(F)c(-c3c(F)cccc3F)n2)O[C@@H]1COS(=O)(=O)c1ccc(C)cc1 ; CC(=O)O[C@H]1CC[C@H](c2ccncc2NC(=O)c2ccc(F)c(-c3c(F)cccc3F)n2)O[C@@H]1COS(=O)(=O)c1ccc(C)cc1]

To a solution of (2R,3S,4R,6R)-6-(3-aminopyridin-4-yl)-2-(tosyloxymethyl)tetrahydro-2H-pyran-3,4-diyl diacetate (1.0 equiv.) in DMF (0.19 M) was added 6-(2,6-difluorophenyl)-5-fluoropicolinic acid (1.2 equiv.), EDCI (1.2 equiv.) and HOAt (1.2 equiv.) and the reaction was stirred at room temperature overnight. The solution was quenched by the addition of water and ethyl acetate. The organic phase was dried with sodium sulfate, filtered and concentrated. The crude material was purified via silica gel column chromatography eluting with ethyl acetate and heptanes (0-50%) to give (2R,3S,4R,6R)-6-(3-(6-(2,6-difluorophenyl)-5-fluoropicolinamido)pyridin-4-yl)-2-(tosyloxymethyl)tetrahydro-2H-pyran-3,4-diyl diacetate and (2R,3S,6R)-6-(3-(6-(2,6-difluorophenyl)-5-fluoropicolinamido)pyridin-4-yl)-2-(tosyloxymethyl)tetrahydro-2H-pyran-3-yl acetate as a brown foam as a mixture of the two products in 60% yield. LC/MS (m/z): 656.3 (MH⁺) and 714.3 (MH⁺) Rₜ=0.87 min.

### Synthesis of ((2R,3S,4R,6R)-6-(3-(6-(2,6-difluorophenyl)-5-fluoropicolinamido)pyridin-4-yl)-3,4-dihydroxytetrahydro-2H-pyran-2-yl)methyl 4-methylbenzenesulfonate and ((2R,3S,6R)-6-(3-(6-(2,6-difluorophenyl)-5-fluoropicolinamido)pyridin-4-yl)-3-hydroxytetrahydro-2H-pyran-2-yl)methyl 4-methylbenzenesulfonate

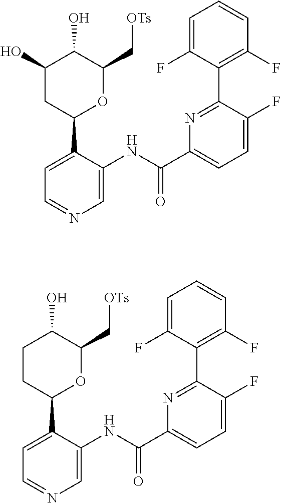 [SMILES: Cc1ccc(S(=O)(=O)OC[C@H]2O[C@@H](c3ccncc3NC(=O)c3ccc(F)c(-c4c(F)cccc4F)n3)C[C@@H](O)[C@@H]2O)cc1 ; Cc1ccc(S(=O)(=O)OC[C@H]2O[C@@H](c3ccncc3NC(=O)c3ccc(F)c(-c4c(F)cccc4F)n3)CC[C@@H]2O)cc1]

To a solution of (2R,3S,4R,6R)-6-(3-(6-(2,6-difluorophenyl)-5-fluoropicolinamido)pyridin-4-yl)-2-(tosyloxymethyl)tetrahydro-2H-pyran-3,4-diyl diacetate and (2R,3S,6R)-6-(3-(6-(2,6-difluorophenyl)-5-fluoropicolinamido)pyridin-4-yl)-2-(tosyloxymethyl)tetrahydro-2H-pyran-3-yl acetate (1.0 equiv.) in EtOH (0.08M) was added potassium carbonate (5 equiv.) and the reaction was stirred at 60° C. overnight. Upon completion, the reaction was concentrated to dryness under vacuo and partitioned between ethyl acetate and water. The organic phase was dried with sodium sulfate, filtered and concentrated. The crude material was purified via silica gel column chromatography eluting with ethyl acetate and heptanes (0-100% ethyl acetate). The pure fractions were concentrated to give ((2R,3S,4R,6R)-6-(3-(6-(2,6-difluorophenyl)-5-fluoropicolinamido)pyridin-4-yl)-3,4-dihydroxytetrahydro-2H-pyran-2-yl)methyl 4-methylbenzenesulfonate in 31% yield. LC/MS (m/z): 630.4 (MH⁺) Rₜ=0.73 min and ((2R,3S,6R)-6-(3-(6-(2,6-difluorophenyl)-5-fluoropicolinamido)pyridin-4-yl)-3-hydroxytetrahydro-2H-pyran-2-yl)methyl 4-methylbenzenesulfonate in 22% yield LC/MS (m/z): 613.6 (MH⁺) Rₜ=0.77 min.

### Synthesis of 5-cyano-N-(4-((2R,4R,5S,6R)-6-(cyanomethyl)-4,5-dihydroxytetrahydro-2H-pyran-2-yl)pyridin-3-yl)-6-(2,6-difluorophenyl)picolinamide

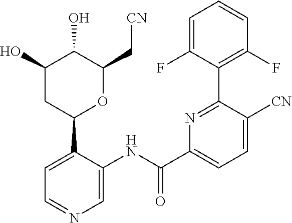 [SMILES: N#CC[C@H]1O[C@@H](c2ccncc2NC(=O)c2ccc(C#N)c(-c3c(F)cccc3F)n2)C[C@@H](O)[C@@H]1O]

To a solution of ((2R,3S,4R,6R)-6-(3-(6-(2,6-difluorophenyl)-5-fluoropicolinamido)pyridin-4-yl)-3,4-dihydroxytetrahydro-2H-pyran-2-yl)methyl 4-methylbenzenesulfonate (1.0 equiv.) in DMSO (0.06M) was added KCN (10 equiv.) and the reaction was heated to 70° C. overnight. The solution was filtered through a PTFE HPLC filter and purified via reverse phase HPLC. The pure fractions were lyophilized for several days to give 5-cyano-N-(4-((2R,4R,5S,6R)-6-(cyanomethyl)-4,5-dihydroxytetrahydro-2H-pyran-2-yl)pyridin-3-yl)-6-(2,6-difluorophenyl)picolinamide as a white fluffy powder in 21% yield (TFA salt). LC/MS (m/z): 492.3 (MH⁺) Rₜ=0.55 min.

### Synthesis of N-(4-((2R,5S,6R)-6-(cyanomethyl)-5-hydroxytetrahydro-2H-pyran-2-yl)pyridin-3-yl)-6-(2,6-difluorophenyl)-5-fluoropicolinamide and 5-cyano-N-(4-((2R,5S,6R)-6-(cyanomethyl)-5-hydroxytetrahydro-2H-pyran-2-yl)pyridin-3-yl)-6-(2,6-difluorophenyl)picolinamide

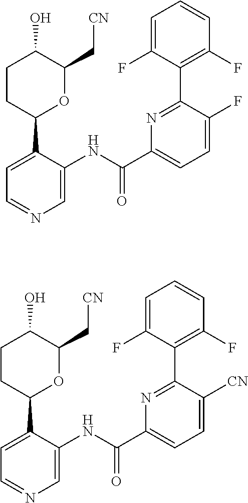 [SMILES: N#CC[C@H]1O[C@@H](c2ccncc2NC(=O)c2ccc(F)c(-c3c(F)cccc3F)n2)CC[C@@H]1O ; N#CC[C@H]1O[C@@H](c2ccncc2NC(=O)c2ccc(C#N)c(-c3c(F)cccc3F)n2)CC[C@@H]1O]

To a solution of ((2R,3S,6R)-6-(3-(6-(2,6-difluorophenyl)-5-fluoropicolinamido)pyridin-4-yl)-3-hydroxytetrahydro-2H-pyran-2-yl)methyl 4-methylbenzenesulfonate (1.0 equiv.) in DMSO (0.05M) was added KCN (10 equiv.) and the reaction was heated to 50° C. for 3 h. Upon checking the reaction by LC/MS formation of the two products was observed. The heat was lowered to 40° C. and the reaction was allowed to go overnight. The solution was then cooled to room temperature, filtered and purified via reverse phase prep-HPLC. The pure fractions were lyophilized for several days to give N-(4-((2R,5S,6R)-6-(cyanomethyl)-5-hydroxytetrahydro-2H-pyran-2-yl)pyridin-3-yl)-6-(2,6-difluorophenyl)-5-fluoropicolinamide in 16% yield (TFA salt) LC/MS (m/z): 469.1 (MH⁺) Rₜ=0.65 min and 5-cyano-N-(4-((2R,5S,6R)-6-(cyanomethyl)-5-hydroxytetrahydro-2H-pyran-2-yl)pyridin-3-yl)-6-(2,6-difluorophenyl)picolinamide in 26% yield (TFA salt) LC/MS (m/z): 476.1 (MH⁺) Rₜ=0.61 min.

### Synthesis of ((2R,3R,4R)-6-(3-nitropyridin-4-yl)-3,4-bis(triisopropylsilyloxy)-3,4-dihydro-2H-pyran-2-yl)methanol

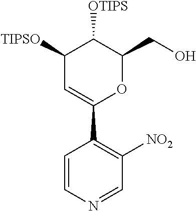

A 0.15 M solution of 4-((2R,3R,4R)-3,4-bis(triisopropylsilyloxy)-2-((triisopropylsilyloxy)methyl)-3,4-dihydro-2H-pyran-6-yl)-3-nitropyridine (1.0 equiv.) in THF was cooled in an ice water bath. Concentrated hydrochloric acid (5 equiv.) was added in a dropwise fashion. The mixture was stirred at ambient temperature for 4.5 hr. The reaction mixture was cooled in an ice water bath, neutralized with saturated aqueous sodium bicarbonate, and extracted with ethyl acetate. The combined organic layers were dried over sodium sulfate, filtered, and concentrated. The crude material was purified by silica gel column chromatography eluting with heptanes and a 0 to 10% ethyl acetate gradient to give ((2R,3R,4R)-6-(3-nitropyridin-4-yl)-3,4-bis(triisopropylsilyloxy)-3,4-dihydro-2H-pyran-2-yl)methanol in 50% yield. LC/MS (m/z): 581.3 (MH⁺), Rₜ=0.62 min (65/95 method). ¹H-NMR (400 MHz, CHLOROFORM-d) δ ppm 0.98-1.16 (m, 42H) 2.44 (dd, 1H) 3.65 (ddd, 1H) 4.10 (d, 1H) 4.13-4.28 (m, 2H) 4.43 (dd, 1H) 5.36 (d, 1H) 7.45 (d, 1H) 8.78 (d, 1H) 8.97 (s, 1H).

### Synthesis of 4-((2R,3R,4R)-3,4-bis(triisopropylsilyloxy)-2-vinyl-3,4-dihydro-2H-pyran-6-yl)pyridin-3-amine

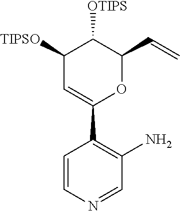

To a 0.10 M solution of 4-((2R,3R,4R)-3,4-bis(triisopropylsilyloxy)-2-vinyl-3,4-dihydro-2H-pyran-6-yl)-3-nitropyridine (1.0 equiv.) in acetic acid was added powdered iron (10.0 equiv.). The reaction was stirred for 1 hr at ambient temperature. The reaction mixture was diluted with ethyl acetate and filtered through Celite. The filtrate was concentrated. The residue was re-dissolved in ethyl acetate and washed with saturated aqueous sodium bicarbonate. The organic phase was dried over sodium sulfate, filtered, and concentrated to give 4-((2R,3R,4R)-3,4-bis(triisopropylsilyloxy)-2-vinyl-3,4-dihydro-2H-pyran-6-yl)pyridin-3-amine as the desired product in 100% yield. LC/MS (m/z): 547.5 (MH⁺) Rₜ=1.09 min (65/95 method).

### Synthesis of 4-((2R,3R,4R)-2-ethyl-3,4-bis(triisopropylsilyloxy)-3,4-dihydro-2H-pyran-6-yl)pyridin-3-amine

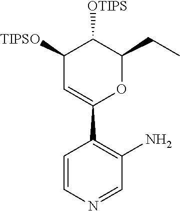

A 0.05 M solution of 4-((2R,3R,4R)-3,4-bis(triisopropylsilyloxy)-2-vinyl-3,4-dihydro-2H-pyran-6-yl)-3-nitropyridine (1.0 equiv.) in ethanol was degassed with argon for 10 min. 10% Lindlar catalyst (0.15 equiv.) was added, and the mixture was stirred under a hydrogen balloon overnight. The reaction was filtered through Celite. The filtrate was concentrated in vacuo to yield 4-((2R,3R,4R)-2-ethyl-3,4-bis(triisopropylsilyloxy)-3,4-dihydro-2H-pyran-6-yl)pyridin-3-amine as the desired product in 100% yield. LC/MS (m/z): 549.5 (MH⁺), Rₜ=1.15 min.

### Synthesis of 4-((4R,5R,6R)-2,3-dideutero-6-ethyl-4,5-bis(triisopropylsilyloxy)tetrahydro-2H-pyran-2-yl)pyridin-3-dideuteroamine

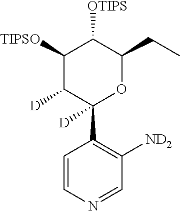

A 0.05 M solution of 4-((2R,3R,4R)-2-ethyl-3,4-bis(triisopropylsilyloxy)-3,4-dihydro-2H-pyran-6-yl)pyridin-3-amine (1.0 equiv.) in methanol-d4 was degassed with argon for 10 min. 10% palladium on carbon (0.15 equiv.) was added, and the mixture was stirred under a deuterium balloon overnight. The reaction was filtered through Celite. The filtrate was concentrated in vacuo to yield 4-((4R,5R,6R)-2,3-dideutero-6-ethyl-4,5-bis(triisopropylsilyloxy)tetrahydro-2H-pyran-2-yl)pyridin-3-dideuteroamine as the desired product in 100% yield. LC/MS (m/z): 554.5 (MH⁺), Rₜ=1.16 min. ¹H-NMR (400 MHz, CHLOROFORM-d) δ ppm 1.00 (t, 3H) 1.03-1.19 (m, 42H) 1.86-1.97 (m, 1H) 2.03 (d, 1H) 3.31-3.40 (m, 1H) 3.57 (t, 1H) 3.98-4.08 (m, 1H) 6.90 (d, 1H) 7.97 (d, 1H) 8.05 (s, 1H).

### Synthesis of 4-((2R,3R,4R)-2-(methoxymethyl)-3,4-bis(triisopropylsilyloxy)-3,4-dihydro-2H-pyran-6-yl)-3-nitropyridine

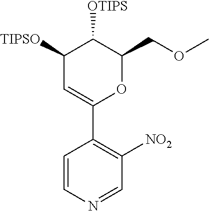

Sodium hydride (2.0 equiv) was added to a 0.16 M solution of ((2R,3R,4R)-6-(3-nitropyridin-4-yl)-3,4-bis(triisopropylsilyloxy)-3,4-dihydro-2H-pyran-2-yl)methanol (1.0 equiv.) in THF. The mixture was stirred at 50° C. for 30 min. Iodomethane (2.1 equiv.) was added. The reaction was stirred for 21 hr at 50° C. The reaction was quenched with saturated aqueous sodium bicarbonate and extracted with ethyl acetate. The combined extracts were dried over sodium sulfate, filtered, and concentrated to give 4-((2R,3R,4R)-2-(methoxymethyl)-3,4-bis(triisopropylsilyloxy)-3,4-dihydro-2H-pyran-6-yl)-3-nitropyridine as the desired product in 100% yield. LC/MS (m/z): 595.6 (MH⁺), Rₜ=0.74 min.

### Synthesis of 4-((2S,4R,5R,6R)-6-(methoxymethyl)-4,5-bis(triisopropylsilyloxy)tetrahydro-2H-pyran-2-yl)pyridin-3-amine

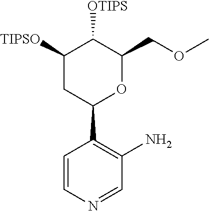

A 0.05 M solution of 4-((2R,3R,4R)-2-(methoxymethyl)-3,4-bis(triisopropylsilyloxy)-3,4-dihydro-2H-pyran-6-yl)-3-nitropyridine (1.0 equiv.) in ethanol was degassed with argon for 10 min. 10% palladium on carbon (0.15 equiv.) was added, and the mixture was stirred under a hydrogen balloon overnight. The reaction was filtered through Celite. The filtrate was concentrated in vacuo to yield 4-((2S,4R,5R,6R)-6-(methoxymethyl)-4,5-bis(triisopropylsilyloxy)tetrahydro-2H-pyran-2-yl)pyridin-3-amine as the desired product in 100% yield. LC/MS (m/z): 567.5 (MH⁺), Rₜ=1.04 min.

### Synthesis of ((2R,3R,4R)-6-(3-aminopyridin-4-yl)-3,4-bis(triisopropylsilyloxy)-3,4-dihydro-2H-pyran-2-yl)methanol and ((2R,3R,4R)-6-(3-aminopyridin-4-yl)-3,4-bis(triisopropylsilyloxy)tetrahydro-2H-pyran-2-yl)methanol

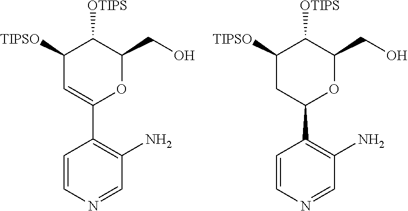

A 0.05 M solution of ((2R,3R,4R)-6-(3-nitropyridin-4-yl)-3,4-bis(triisopropylsilyloxy)-3,4-dihydro-2H-pyran-2-yl)methanol (1.0 equiv.) in ethanol was degassed with argon for 10 min. 10% palladium on carbon (0.10 equiv.) was added, and the mixture was stirred under a hydrogen balloon for 3 days. The reaction was filtered through Celite. The filtrate was concentrated in vacuo. The residue was purified by silica gel column chromatography eluting with heptanes and a 25-75% ethyl acetate gradient to yield ((2R,3R,4R)-6-(3-aminopyridin-4-yl)-3,4-bis(triisopropylsilyloxy)-3,4-dihydro-2H-pyran-2-yl)methanol in 41% yield and ((2R,3R,4R)-6-(3-aminopyridin-4-yl)-3,4-bis(triisopropylsilyloxy)tetrahydro-2H-pyran-2-yl)methanol in 47% yield. LC/MS (m/z): 551.4 (MH⁺), Rₜ=0.92 min. LC/MS (m/z): 553.4 (MH⁺), Rₜ=0.94 min.

### Synthesis of 4-((2S,3R,4R)-2-(chloromethyl)-3,4-bis(triisopropylsilyloxy)-3,4-dihydro-2H-pyran-6-yl)pyridin-3-amine

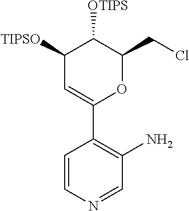

To a 0.2 M solution of ((2R,3R,4R)-6-(3-aminopyridin-4-yl)-3,4-bis(triisopropylsilyloxy)-3,4-dihydro-2H-pyran-2-yl)methanol (1.0 equiv.) in pyridine was added triphenylphosphine (3.0 equiv.) and carbon tetrachloride (1.5 equiv.). The mixture was stirred at ambient temperature for 18 hr. The reaction mixture was concentrated under reduced pressure. The residue was purified by silica gel column chromatography eluting with heptane and a 25-75% ethyl acetate gradient to give 4-((2S,3R,4R)-2-(chloromethyl)-3,4-bis(triisopropylsilyloxy)-3,4-dihydro-2H-pyran-6-yl)pyridin-3-amine as the desired product in 45% yield. LC/MS (m/z): 569.1 (MH⁺), Rₜ=0.95 min.

### Synthesis of 4-((2R,4R,5R,6S)-6-(chloromethyl)-4,5-bis(triisopropylsilyloxy)tetrahydro-2H-pyran-2-yl)pyridin-3-amine

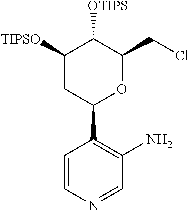

To a 0.2 M solution of ((2R,3R,4R)-6-(3-aminopyridin-4-yl)-3,4-bis(triisopropylsilyloxy)tetrahydro-2H-pyran-2-yl)methanol (1.0 equiv.) in pyridine was added triphenylphosphine (3.0 equiv.) and carbon tetrachloride (1.5 equiv.). The mixture was stirred at ambient temperature for 18 hr. The reaction mixture was concentrated under reduced pressure. The residue was purified by silica gel column chromatography eluting with heptane and a 25-75% ethyl acetate gradient to give 4-((2R,4R,5R,6S)-6-(chloromethyl)-4,5-bis(triisopropylsilyloxy)tetrahydro-2H-pyran-2-yl)pyridin-3-amine as the desired product in 70% yield. LC/MS (m/z): 571.1 (MH⁺), Rₜ=0.98 min. ¹H-NMR (400 MHz, CHLOROFORM-d) δ ppm 1.05-1.17 (m, 42H) 2.08-2.21 (m, 1H) 2.28 (ddd, 1H) 3.67-3.83 (m, 3H) 3.86-3.94 (m, 1H) 4.08 (dt, 1H) 4.60 (dd, 1H) 6.87 (d, 1H) 7.98 (d, 1H) 8.06 (s, 1H).

### Synthesis of (2R,3R,4R)-6-(3-nitropyridin-4-yl)-3,4-bis(triisopropylsilyloxy)-3,4-dihydro-2H-pyran-2-carbonitrile

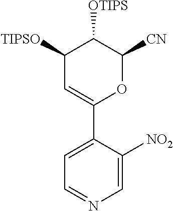

To a round-bottom flask containing (2S,3R,4R)-6-(3-nitropyridin-4-yl)-3,4-bis(triisopropylsilyloxy)-3,4-dihydro-2H-pyran-2-carbaldehyde in water/MeOH (1:5, 0.24 M) was added hydroxyamine (2 equiv) and sodium methanolate (2.2 equiv) in MeOH. The reaction mixture was capped and heated at 60° C. in an oil bath for 3 hours. The volatiles were removed under vacuo. The residue was dissolved in pyridine (0.6 M) and the solution was added dropwise to a mixture of pyridine (87 equiv) and acetic anhydride (34 equiv). After stirring at room temperature overnight, the reaction mixture was cooled to 0° C., quenched with Sat. NaHCO₃ and extracted with DCM. The organic layer was washed with H₂O and sat. NaCl. The organic layer was dried over Na₂SO₄, filtered and concentrated. To the crude residue in Acetic acid (0.18 M) was added sodium acetate (1 equiv). The reaction mixture was heated at 100° C. for 2 hours. The volatiles were removed under vacuo. The residue was dissolved in EtOAc and washed with NaHCO₃(sat.) and NaCl_{(sat.)}. The organic layer was dried over Na₂SO₄, filtered and concentrated. The crude was purified by column chromatography on silica gel with EtOAc/Hexane (1/9) to yield (2R,3R,4R)-6-(3-nitropyridin-4-yl)-3,4-bis(triisopropylsilyloxy)-3,4-dihydro-2H-pyran-2-carbonitrile in 48.6% yield over three steps. ¹H NMR (400 MHz, CHLOROFORM-d) δ ppm 1.03-1.19 (m, 42H) 4.20-4.31 (m, 2H) 5.02 (s, 1H) 5.53-5.60 (m, 1H) 7.43 (d, 1H) 8.79-8.85 (m, 1H) 9.02-9.07 (m, 1H). LC-MS (m/z): 576.4 (MH⁺) Rₜ=0.55 min. (95/95 method).

### Synthesis of (2R,3R,4R)-6-(3-aminopyridin-4-yl)-3,4-bis(triisopropylsilyloxy)-3,4-dihydro-2H-pyran-2-carbonitrile

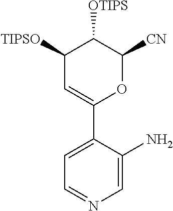

To a round-bottom flask containing (2R,3R,4R)-6-(3-nitropyridin-4-yl)-3,4-bis(triisopropylsilyloxy)-3,4-dihydro-2H-pyran-2-carbonitrile was added AcOH (0.1 M) and iron (10 equiv). The reaction mixture was stirred at room temperature for 16 hours. The reaction mixture was filtered. The filtrate was concentrated to dryness, diluted with EtOAc, washed with NaHCO_{3(sat.)} and NaCl_{(sat.)}. The organic layer was dried over Na₂SO₄, filtered and concentrated to afford (2R,3R,4R)-6-(3-aminopyridin-4-yl)-3,4-bis(triisopropylsilyloxy)-3,4-dihydro-2H-pyran-2-carbonitrile in 96% yield. LC-MS (m/z): 546.2 (MH⁺) Rₜ=0.90 min (65/95 method).

### Synthesis of (2R,3R,4R,6R)-6-(3-aminopyridin-4-yl)-3,4-bis(triisopropylsilyloxy)tetrahydro-2H-pyran-2-carbonitrile

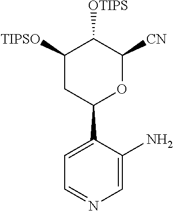

A solution of (2R,3R,4R)-6-(3-nitropyridin-4-yl)-3,4-bis(triisopropylsilyloxy)-3,4-dihydro-2H-pyran-2-carbonitrile (1 equiv) in MeOH/EtOAc (1:1, 0.08 M) was degassed with nitrogen. 10% Pd—C (0.2 equiv) was added to the mixture and the solution was stirred under a hydrogen balloon for 45 hours at room temperature. The reaction mixture was filtered over celite and the filtrate was concentrated. The crude was purified by column chromatography on silica gel with EtOAc/Hexane (2/3) to yield (2R,3R,4R,6R)-6-(3-aminopyridin-4-yl)-3,4-bis(triisopropylsilyloxy)tetrahydro-2H-pyran-2-carbonitrile in 63% yield. LC-MS (m/z): 548.2 (MH⁺), Rₜ=0.97 min (65/95 method). ¹H NMR (400 MHz, CHLOROFORM-d) δ ppm 1.04-1.33 (m, 42H) 2.13-2.31 (m, 2H) 4.09 (d, 2H) 4.16 (s, 2H) 4.39 (d, 1H) 4.63 (dd, 1H) 6.90 (d, 1H) 7.99 (d, 1H) 8.08 (s, 1H).

### Synthesis of (E)-N,N-dimethyl-2-(3-nitropyridine-4-yl)ethanamine

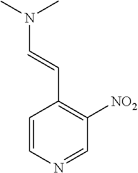

To a solution of 4-methyl-3-nitropyridine (1.0 equiv.) in DMF (5.5 M) was added 1,1-dimethoxy-N,N-dimethylmethaneamine (1.0 equiv.) and the solution was allowed to stir at 120° C. for 13 hrs. The reaction was cooled to room temperature, poured onto crushed ice and stirred for 5 min. The red solid was filtered and washed with cold water. The solid was recrystallized form hot MeOH to yield (E)-N,N-dimethyl-2-(3-nitropyridine-4-yl)ethanamine as the desired product in 45% yield. LC/MS (m/z): 194.0 (MH⁺), Rₜ=0.39 min.

### Synthesis of 3-nitroisonicotinaldehyde

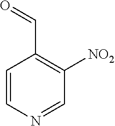 [SMILES: O=Cc1ccncc1[N+](=O)[O-]]

To a solution of (E)-N,N-dimethyl-2-(3-nitropyridine-4-yl)ethanamine (1.0 equiv.) in THF/Water (1:1) (0.5 M) at 0° C. was added sodium periodate (3.0 equiv.). The reaction mixture was stirred at 0° C. for 16 hrs. The solid was filtered and rinsed with EtOAc (200 mL). The solution was diluted further with EtOAc (400 mL) and was washed with NaHCO_{3(sat.)} (3×150 mL) and NaCl (sat, 150 mL). The combined aqueous were back extracted with additional EtOAc (2×200 mL) and the combined organics were dried over MgSO₄, filtered and the volatiles were removed in vacuo. Purification was completed by silica gel column chromatography via ISCO Combi-flash Rf system (80 g column, 60 mL/min, 0-60% EtOAc/heptanes gradient) to yield 3-nitroisonicotinaldehyde as the desired product in 59%. ¹H NMR (400 MHz, CHLOROFORM-d) δ ppm 7.78 (d, 1H) 9.10 (d, 1H) 9.46 (s, 1H) 10.56 (s, 1H).

### Synthesis of (E)-3-ethylpent-3-en-2-one

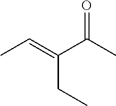

To a solution of 3-ethylpent-lyn-3-ol (1.0 equiv.) in CCl₄ (1.0 M) was added Nafion-H(SCA 13 or NR 50) (1.0 equiv.). The reaction mixture was heated at reflux for 16 hrs. The reaction was filtered and the volatiles were removed in vacuo. The crude was purified by distillation, b.p. 55°-60° C. at 50 torr to yield (E)-3-ethylpent-3-en-2-one as the desired product in 51%. LC/MS (m/z): 154.1, 113.0 (MH⁺), Rₜ=0.67 min. ¹H NMR (400 MHz, CHLOROFORM-d) δ ppm 0.93 (t, 3H) 1.88 (d, 3H) 2.27-2.34 (m, 5H) 6.71 (q, 1H).

### Synthesis of (E)-(3-ethylpenta-1,3-dien-2-yloxy)trimethylsilane

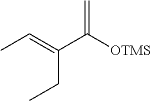

To a solution of LiHMDS (1.1 equiv.) in THF (0.15 M mL) cooled at −78° C. (internal thermometer) under N₂ was added (E)-3-ethylpent-3-en-2-one (1.0 equiv.) slowly into the base solution over 10 min, keeping the internal temperature <−70° C. 5 min later was added TMS-Cl (2 equiv.) as a slow stream. The reaction mixture was stirred for 5 hrs at −78° C. The reaction was poured into ice-cold saturated NaHCO₃ (250 mL) and Heptanes (500 mL). The mixture was allowed to warm up to room temperature prior to separation. The organics were washed with NaHCO_{3(sat.)} (2×250 ml), dried over Na₂SO₄, filtered and the volatiles were removed in vacuo. The crude liquid was purified by distillation, b.p. 74°-77° C. at 40 ton to yield (E)-(3-ethylpenta-1,3-dien-2-yloxy)trimethylsilaneas the desired product in 85% yield. ¹H NMR (400 MHz, CHLOROFORM-d) δ ppm 0.02-0.04 (m, 9H) 0.83 (t, 3H) 1.53 (d, 3H) 2.05 (q, 2H) 4.08 (s, 1H) 4.27 (s, 1H) 5.79 (q, 1H).

### Synthesis of cis (+/−)-4-(5-ethyl-6-methyl-4-(trimethylsilyoxy)-3,6-dihydro-2H-pyran-2-yl)-3-nitropyridine

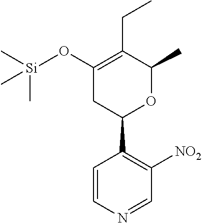 [SMILES: CCC1=C(O[Si](C)(C)C)C[C@H](c2ccncc2[N+](=O)[O-])O[C@@H]1C]

A solution of 3-nitroisonicotinaldehyde (1.5 equiv.), (E)-(3-ethylpenta-1,3-dien-2-yloxy)trimethylsilane (1.0 equiv.), and tris(6,6,7,7,8,8,8-heptafluoro-2,2-dimethyl-3,5-octanedionato) europium (0.05 equiv.) were dissolved in CHCl₃ (0.20 M) and stirred in a flame-dried round-bottom flask at 60° C. under an atmosphere of nitrogen for 16 hrs. The reaction was quenched with water and the product was extracted in the organic layer. The organics were dried over Na₂SO₄, filtered and the volatiles were removed in vacuo. Purification was completed by column chromatography via a ISCO Combi-flash Rf system (220 g column, 150 mL/min, 0-40% EtOAc/heptanes gradient) to yield cis (+/−)-4-(5-ethyl-6-methyl-4-(trimethylsilyoxy)-3,6-dihydro-2H-pyran-2-yl)-3-nitropyridine as the desired product in 48% yield. LC/MS (m/z): 337.0 (MH⁺), Rₜ=1.27 min. ¹H NMR (400 MHz, CHLOROFORM-d) δ ppm 0.14-0.27 (m, 9H) 1.00 (t, 3H) 1.35 (d, 3H) 1.92 (ddd, 1H) 2.20-2.29 (m, 1H) 2.30-2.42 (m, 1H) 2.44-2.51 (m, 1H) 4.42-4.49 (m, 1H) 5.20 (dd, 2.93 Hz, 1H) 7.85 (d, 1H) 8.89 (d, 1H) 9.23 (s, 1H).

### Synthesis of (+/−)-3-ethyl-3-hydroxy-2-methyl-6-(3-nitropyridine-4-yl)dihydro-2H-pyran-4-(3H)-one+C3-epimeric (+/−)-3-ethyl-3-hydroxy-2-methyl-6-(3-nitropyridine-4-yl)dihydro-2H-pyran-4-(31)-one

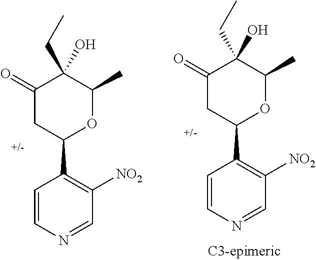 [SMILES: CC[C@]1(O)C(=O)C[C@H](c2ccncc2[N+](=O)[O-])O[C@@H]1C ; CC[C@@]1(O)C(=O)C[C@H](c2ccncc2[N+](=O)[O-])O[C@@H]1C]

To a solution of (+/−)-4-(5-ethyl-6-methyl-4-(trimethylsilyoxy)-3,6-dihydro-2H-pyran-2-yl)-3-nitropyridine (1.0 equiv.) in DCM (0.5 M) was added 0.5 equiv of 3,3-dimethyldioxirane as a solution in acetone at 0° C. and allowed to stir for 10 mins. An additional 0.25 eq of 3,3-dimethyldioxirane was added and allowed to stir for an additional 10 min. The final 0.25 eq of 3,3-dimethyldioxirane was added and the ice bath was removed allowing the reaction to stir for an additional 10 min. To the reaction was added 10 mL of cyclohexene; the reaction stirred for 10 mins and the volatiles were removed in vacuo. The residue was taken up in THF (50 mL) and acidified with 5 mL of 2 M HCl and the reaction stirred for 15 min. The solution was basified with 2 M NaOH to ˜pH=9. The product was extracted in EtOAc, dried over MgSO₄, filtered and the volatiles were removed in vacuo. Purification was completed by column chromatography via ISCO Combi-flash Rf system (120 g column, 85 mL/min, 0-60% EtOAc/Heptanes gradient) to yield cis (+/−)-3-ethyl-3-hydroxy-2-methyl-6-(3-nitropyridine-4-yl)dihydro-2H-pyran-4-(3H)-one in 41% yield. LC/MS (m/z): 281.0 (MH⁺), Rₜ=0.65 min. ¹H NMR (400 MHz, CHLOROFORM-d) δ 0.78 (t, 3H) 1.39 (d, 3H) 1.85-1.96 (m, 1H) 2.00-2.12 (m, 1H) 2.56-2.64 (m, 1H) 3.08 (dd, 1H), 3.88 (s, 1H) 5.33 (dd, 1H) 7.88 (d, 1H) 8.90 (d, 1H) 9.23 (s, 1H). The C-3 epimeric (+/−)-3-ethyl-3-hydroxy-2-methyl-6-(3-nitropyridine-4-yl)dihydro-2H-pyran-4-(3H)-one was obtained in 47% yield. LC/MS (m/z): 281.0 (MH⁺), Rₜ=0.66 min. ¹H NMR (400 MHz, CHLOROFORM-d) δ ppm 0.94 (t, 3H) 1.37 (d, 3H) 1.62-1.72 (m, 1H) 1.84-1.95 (m, 1H) 2.76 (s, 1H) 2.86 (dd, 1H) 3.08 (dd, 1H) 4.02 (q, 1H) 5.51 (dd, 1H) 7.78 (d, 1H) 8.87 (d, 1H) 9.22 (s, 1H).

### Synthesis of (+/−)-3-ethyl-2-methyl-6-(3-nitropyridin-4-yl)tetrahydro-2H-pyran-3,4-diol

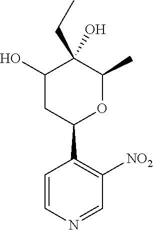 [SMILES: CC[C@]1(O)C(O)C[C@H](c2ccncc2[N+](=O)[O-])O[C@@H]1C]

To a solution of (+/−)-3-ethyl-3-hydroxy-2-methyl-6-(3-nitropyridine-4-yl)dihydro-2H-pyran-4-(3H)-one (1.0 equiv.) in EtOH (0.18 M) at 0° C. was added sodium borohydride (1.2 equiv.). The reaction mixture was allowed to stir for 5 hr warming to room temperature. The reaction was quenched with water and the volatiles were removed in vacuo; the residue was taken up into EtOAc and washed with brine. The organics were dried over Na₂SO₄, filtered, and the volatiles were removed in vacuo to yield (+/−)-3-ethyl-2-methyl-6-(3-nitropyridin-4-yl)tetrahydro-2H-pyran-3,4-diol as a mixture of diastereomers (6:1) in 71% yield. LC/MS (m/z): 283.1 (MH⁺), Rₜ=0.56 min.

### Synthesis of (+/−)-3-ethyl-3-hydroxy-2-methyl-6-(3-nitropyridin-4-yl)tetrahydro-2H-pyran-4-yl acetate

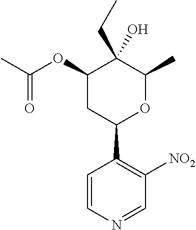 [SMILES: CC[C@@]1(O)[C@@H](C)O[C@@H](c2ccncc2[N+](=O)[O-])C[C@H]1OC(C)=O]

To a solution of (+/−)-3-ethyl-2-methyl-6-(3-nitropyridin-4-yl)tetrahydro-2H-pyran-3,4-diol (1.0 equiv.) in pyridine (0.15 M) was added acetic anhydride (3.0 equiv.). The reaction mixture was allowed to stir for 5 hr warming to room temperature. The reaction was quenched with water and the product was extracted in EtOAc and washed with brine. The organics were dried over Na₂SO₄, filtered, and volatiles were removed in vacuo. Purification was completed by silica gel column chromatography via ISCO Combi-flash Rf system (80 g column, 60 mL/min, 0-60% EtOAc/heptanes gradient) to yield (+/−)-3-ethyl-3-hydroxy-2-methyl-6-(3-nitropyridin-4-yl)tetrahydro-2H-pyran-4-yl acetate as the desired product in 87% yield. LC/MS (m/z): 325.1 (MH⁺), Rₜ=0.76 min. ¹H NMR (400 MHz, CHLOROFORM-d) δ ppm 1.08 (t, 3H) 1.30 (d, 3H) 1.67-1.90 (m, 3H) 2.09-2.12 (m, 2H) 2.41 (ddd, 1H) 3.60 (q, 1H) 5.10 (dd, 1H) 5.23 (dd, 1H) 7.80 (d, 1H) 8.84 (d, 1H) 9.18 (s, 1H).

### Synthesis of (2R,3R,4R,6R)-6-(3-aminopyridin-4-yl)-3-ethyl-3-hydroxy-2-methyltetrahydro-2H-pyran-4-yl acetate and (2S,3S,4S,6S)-6-(3-aminopyridin-4-yl)-3-ethyl-3-hydroxy-2-methyltetrahydro-2H-pyran-4-yl acetate

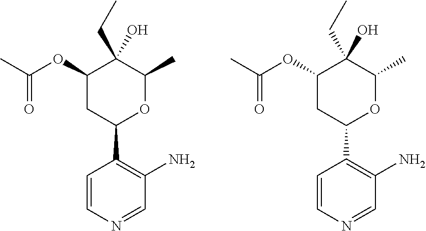 [SMILES: CC[C@@]1(O)[C@@H](C)O[C@@H](c2ccncc2N)C[C@H]1OC(C)=O ; CC[C@]1(O)[C@H](C)O[C@H](c2ccncc2N)C[C@@H]1OC(C)=O]

A solution of (+/−)-3-ethyl-3-hydroxy-2-methyl-6-(3-nitropyridin-4-yl)tetrahydro-2H-pyran-4-yl acetate (1.0 equiv.) in acetic acid (0.1 M) was degassed with nitrogen for 20 min. Iron dust (10 equiv.) was added to the mixture and the solution was stirred in a closed system at room temperature for 6 hours. The reaction mixture was diluted with DCM and methanol (50 mL, 1:1) and filtered through celite. The filtrate was concentrated in vacuo and re-dissolved in ethyl acetate. The organic was washed with NaHCO_{3(sat.)}, dried over Na₂SO₄, filtered, and the volatiles were removed in vacuo. Purification was completed via chiral HPLC (Heptanes/EtOH=75/25, 1 mL/min, AD-H column) to yield (2R,3R,4R,6R)-6-(3-aminopyridin-4-yl)-3-ethyl-3-hydroxy-2-methyltetrahydro-2H-pyran-4-yl acetate (21% yield, >99% ee) and (2S,3S,4S,6S)-6-(3-aminopyridin-4-yl)-3-ethyl-3-hydroxy-2-methyltetrahydro-2H-pyran-4-yl acetate (23% yield, >99% ee). ¹H NMR (400 MHz, CHLOROFORM-d) δ ppm 0.89 (d, 1H) 1.04-1.11 (m, 3H) 1.30 (dd, 3H) 1.71-1.83 (m, 1H) 1.84-1.95 (m, 1H) 2.11-2.17 (m, 5H) 2.65 (br. s., 1H) 3.57 (dd, 1H) 4.21 (br. s., 2H) 4.57-4.64 (m, 1H) 5.00 (ddd, 1H) 6.94 (d, 1H) 7.97-8.02 (m, 1H) 8.06 (d, 1H).

### Synthesis of (+/−)-3-ethyl-2-methyl-6-(3-nitropyridin-4-yl)tetrahydro-2H-pyran-3,4-diol

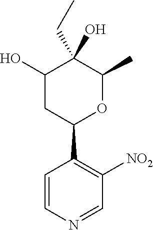 [SMILES: CC[C@@]1(O)C(O)C[C@H](c2ccncc2[N+](=O)[O-])O[C@@H]1C]

To a solution of (+/−)-3-ethyol-3-hydroxy-2-methyl-6-(3-nitropyridine-4-yl)dihydro-2H-pyran-4-(3H)-one (1.0 equiv.) in EtOH (0.18 M) at 0° C. was added sodium borohydride (1.2 equiv.). The reaction mixture was allowed to stir for 5 hr warming to room temperature. The reaction was quenched with water and volatiles were removed in vacuo; the residue was taken up into EtOAc and washed with brine. The organics were dried over Na₂SO₄, filtered, and the volatiles were removed in vacuo to yield (+/−)-3-ethyl-2-methyl-6-(3-nitropyridin-4-yl)tetrahydro-2H-pyran-3,4-diol as the desired product in 70% yield. No further purification was needed. LC/MS (m/z): 283.1 (MH⁺) Rₜ=0.54 min.

### Synthesis of (+/−)-4-(tert-butyldimethylsilyoxy)-3-theyl-2-methyl-6-(3-nitropyridin-4-yl)tetrahydro-2H-pyran-3-ol

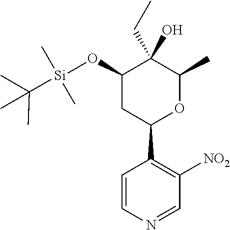 [SMILES: CC[C@]1(O)[C@@H](C)O[C@@H](c2ccncc2[N+](=O)[O-])C[C@H]1O[Si](C)(C)C(C)(C)C]

To a solution of (+/−)-3-ethyl-2-methyl-6-(3-nitropyridin-4-yl)tetrahydro-2H-pyran-3,4-diol (1.0 equiv.) in DCM (1.0 M) was added 2,6-lutidine (2.5 equiv.) and TBDMSOTf (1.5 equiv.). The reaction was allowed to stir at room temperature for 5 hr. The reaction was quenched with NaHCO₃₍ₛₐₜ₎ (25 mL) and then poured onto DCM (50 mL). The organic layer was then washed with brine, and 10% CuSO₄ (until CuSO₄ solution is unchanged ca. 3×50 mL). The organic was then dried over Na₂SO₄, filtered, and the volatiles were removed in vacuo. Purification was completed by silica gel column chromatography via ISCO Combi-flash Rf system (40 g column, 40 mL/min, 0-50% EtOAc/Heptanes gradient) to yield (+/−)-4-(tert-butyldimethylsilyoxy)-3-ethyl-2-methyl-6-(3-nitropyridin-4-yl)tetrahydro-2H-pyran-3-ol as the desired product in 54% yield. LC/MS (m/z): 397.3 (MH⁺), Rₜ=1.28 min. ¹H NMR (400 MHz, CHLOROFORM-d) δ ppm 0.12 (s, 3H) 0.18 (s, 3H) 0.96-0.99 (m, 12H) 1.17 (d, 3H) 1.37-1.48 (m, 1H) 1.52-1.64 (m, 2H) 1.91-2.06 (m, 2H) 3.98 (t, 1H) 5.42 (dd, 1H) 7.69 (d, 1H) 8.78 (d, 1H) 9.06 (s, 1H).

### Synthesis of (2R,3S,4R,6R)-6-(3-aminopyridin-4-yl)-4-(tert-butyldimethylsilyoxy)-3-ethyl-2-methyltetrahysdro-2H-pyran-3-ol and (2S,3R,4S,6S)-6-(3-aminopyridin-4-yl)-4-(tert-butyldimethylsilyoxy)-3-ethyl-2-methyltetrahysdro-2H-pyran-3-ol

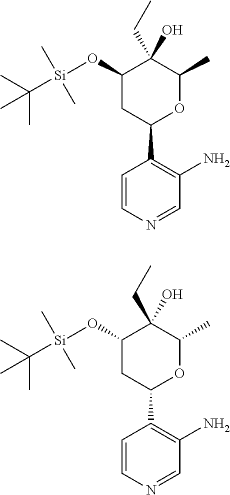

A solution of (+/−)-4-(tert-butyldimethylsilyoxy)-3-theyl-2-methyl-6-(3-nitropyridin-4-yl)tetrahydro-2H-pyran-3-ol (1.0 equiv.) in EtOH (0.15 M) was degassed with nitrogen for 20 min. 10% Pd/C (0.2 equiv.) was added to the mixture and the solution was stirred under a hydrogen balloon for 16 hours. The reaction was filtered, and the volatiles were removed in vacuo. Purification was completed via chiral HPLC (Heptanes/EtOH=90/10, 1 mL/min, AD-H column) to yield (2R,3S,4R,6R)-6-(3-aminopyridin-4-yl)-4-(tert-butyldimethylsilyoxy)-3-ethyl-2-methyltetrahysdro-2H-pyran-3-ol (18% yield, 99% ee) and (2S,3R,4S,6S)-6-(3-aminopyridin-4-yl)-4-(tert-butyldimethylsilyoxy)-3-ethyl-2-methyltetrahysdro-2H-pyran-3-ol (16% yield, 99% ee). ¹H NMR (400 MHz, CHLOROFORM-d) δ ppm 0.09-0.14 (m, 3H) 0.17-0.20 (m, 3H) 0.92-1.01 (m, 12H) 1.15-1.21 (m, 3H) 1.37-1.48 (m, 1H) 1.52-1.65 (m, 2H) 1.91-2.06 (m, 2H) 3.98 (s, 1H) 5.42 (d, 1H) 7.69 (d, 1H) 8.78 (d, 1H) 9.06 (s, 1H).

### Synthesis of triethyl((2Z,4E)-hexa-2,4-dien-3-yloxy)silane

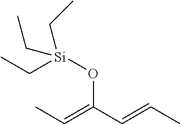

To a round bottom flask, LiHMDS in THF (1.4 equiv) was added at room temperature, which was cooled down to −78° C. The solution of (E)-hex-4-en-3-one (1.0 equiv) in THF (2 M) was slowly introduced to the reaction mixture for 15 min. Followed by addition of chlorotriethylsilane (1.5 equiv) for 15 min, the reaction mixture was stirred at −78° C. for 30 min and then allowed to warm to room temperature. The reaction mixture was poured into cold NaHCO₃ aqueous solution, which was extracted with heptane. The organic layer was washed with water and brine, dried over anhydrous Na₂SO₄, filtered, and dried in vacuo. The crude yellow oil was purified by vacuum distillation to yield triethyl((2Z,4E)-hexa-2,4-dien-3-yloxy)silane (80%) as colorless oil ¹H-NMR (400 MHz, CDCl₃): δ 5.85 (m, 1H), 5.77 (m, 1H), 4.70 (m, 1H), 1.75 (m, 3H), 1.64 (m, 3H), 1.00 (m, 9H), 0.70 (m, 6H).

### Synthesis of (+/−)-4-((2R,3R,6R)-3,6-dimethyl-4-(triethylsilyloxy)-3,6-dihydro-2H-pyran-2-yl)-3-nitropyridine

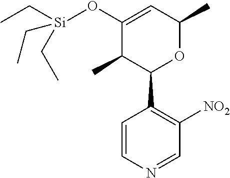 [SMILES: CC[Si](CC)(CC)OC1=C[C@@H](C)O[C@@H](c2ccncc2[N+](=O)[O-])[C@H]1C]

To a solution of triethyl((2Z,4E)-hexa-2,4-dien-3-yloxy)silane (1.5 equiv.) and 3-nitroisonicotinaldehyde (1.0 equiv.) in CHCl₃ (1.2 M) was added Eu(fod)₃ (0.05 equiv.). The reaction mixture was gently refluxed for 2 h. After cooling down, the volatile materials were removed in vacuo. The crude product was purified (10 to 20% EtOAc in heptane) by silica chromatography to give (+/−)-4-((2R,3R,6R)-3,6-dimethyl-4-(triethylsilyloxy)-3,6-dihydro-2H-pyran-2-yl)-3-nitropyridine (11.07 g, 87%). LCMS (m/z): 365.1 (MH⁺), Rₜ=1.02 min. ¹H-NMR (400 MHz, CDCl₃): δ 9.27 (bs, 1H), 8.80 (m, 1H), 7.88 (m, 1H), 5.43 (m, 1H), 4.77 (m, 1H), 4.42 (m, 1H), 2.44 (m, 1H), 1.31 (m, 3H), 1.00 (m, 9H), 0.76 (m, 3H), 0.73 (m, 6H).

### Synthesis of (+/−)-(2R,3R,5R,6R)-3-hydroxy-2,5-dimethyl-6-(3-nitropyridin-4-yl)dihydro-2H-pyran-4(3H)-one and (2R,3S,5R,6R)-3-hydroxy-2,5-dimethyl-6-(3-nitropyridin-4-yl)dihydro-2H-pyran-4(3H)-one

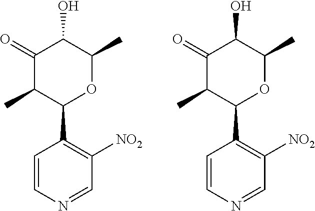 [SMILES: C[C@H]1O[C@@H](c2ccncc2[N+](=O)[O-])[C@@H](C)C(=O)[C@@H]1O ; C[C@H]1O[C@@H](c2ccncc2[N+](=O)[O-])[C@@H](C)C(=O)[C@H]1O]

A solution of (+/−)-4-((2R,3R,6R)-3,6-dimethyl-4-(triethylsilyloxy)-3,6-dihydro-2H-pyran-2-yl)-3-nitropyridine (1.0 equiv.), sodium bicarbonate (5.0 equiv.), acetone (10.0 equiv.), water (0.2 M) and ethyl acetate (0.2M) was vigorously stirred at room temperature. To this, a solution of OXONE (1.0 equiv.) in water (45 mL) was slowly added via dropping funnel for 1 h 30 min. After addition, the reaction mixture was allowed to stir at room temperature for 2 h. After diluted with EtOAc, the organic phase was separated and washed with brine. After the organic phase was dried over anhydrous sodium sulfate, filtered and evaporated in vacuo, the crude reaction mixture, (+/−)-4-((1R,2R,4R,5R,6R)-2,5-dimethyl-6-(triethylsilyloxy)-3,7-dioxabicyclo[4.1.0]heptan-4-yl)-3-nitropyridine and (+/−)-4-((1S,2R,4R,5R,6S)-2,5-dimethyl-6-(triethylsilyloxy)-3,7-dioxabicyclo[4.1.0]heptan-4-yl)-3-nitropyridine, was obtained in 1:1 ratio (based on ¹H-NMR of crude product). The crude product was dissolved in THF (30 mL) and MeOH (15 mL), to this, 3 N HCl aqueous solution (15 mL) was added. After stirring for 1 h, the reaction mixture was neutralized with saturated NaHCO₃ solution and extracted with EtOAc (100 mL), which was then washed with brine (100 mL). The separated organic layer was dried over anhydrous sodium sulfate, filtered, and concentrated in vacuo. The crude product was purified by silica column chromatography to afford a mixture of (+/−)-(2R,3R,5R,6R)-3-hydroxy-2,5-dimethyl-6-(3-nitropyridin-4-yl)dihydro-2H-pyran-4(3H)-one and (+/−)-(2R,3S,5R,6R)-3-hydroxy-2,5-dimethyl-6-(3-nitropyridin-4-yl)dihydro-2H-pyran-4(3H)-one (˜1.9 to 1 ratio, 54.5%). LCMS (m/z): 266.7 (MH⁺), Rₜ=0.56 min, 249.0 (MH+−18), Rₜ=0.59 min.

### Synthesis of (+/−)-(2R,3S,4R,5S,6R)-2,5-dimethyl-6-(3-nitropyridin-4-yl)tetrahydro-2H-pyran-3,4-diol and (+/−)-(2R,3R,4R,5S,6R)-2,5-dimethyl-6-(3-nitropyridin-4-yl)tetrahydro-2H-pyran-3,4-diol

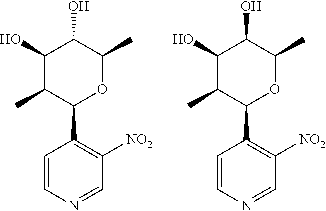 [SMILES: C[C@H]1[C@@H](O)[C@H](O)[C@@H](C)O[C@H]1c1ccncc1[N+](=O)[O-] ; C[C@H]1[C@@H](O)[C@@H](O)[C@@H](C)O[C@H]1c1ccncc1[N+](=O)[O-]]

To a solution of (+/−)-(2R,3R,5R,6R)-3-hydroxy-2,5-dimethyl-6-(3-nitropyridin-4-yl)dihydro-2H-pyran-4(3H)-one and (+/−)-(2R,3S,5R,6R)-3-hydroxy-2,5-dimethyl-6-(3-nitropyridin-4-yl)dihydro-2H-pyran-4(3H)-one (1.0 equiv.) in EtOH (0.1 M) was added sodium borohydride (1.1 equiv.) at 0° C. The reaction mixture was stirred and slowly warmed up to room temperature for 2 h. The mixture was diluted with EtOAc and washed with water and brine, dried over sodium sulfate, filtered and concentrated in vacuo. The inseparable crude reaction mixture of (+/−)-(2R,3S,4R,5S,6R)-2,5-dimethyl-6-(3-nitropyridin-4-yl)tetrahydro-2H-pyran-3,4-diol and (+/−)-(2R,3R,4R,5S,6R)-2,5-dimethyl-6-(3-nitropyridin-4-yl)tetrahydro-2H-pyran-3,4-diol was carried over for the next step without purification. LCMS (m/z): 269.0 (MH⁺), Rₜ=0.47 min and 0.48 min.

### Synthesis of (+/−)-4-((2R,3R,4R,5R,6R)-3,6-dimethyl-4,5-bis(triethylsilyloxy)tetrahydro-2H-pyran-2-yl)-3-nitropyridine and (+/−)-4-((2R,3R,4R,5S,6R)-3,6-dimethyl-4,5-bis(triethylsilyloxy)tetrahydro-2H-pyran-2-yl)-3-nitropyridine

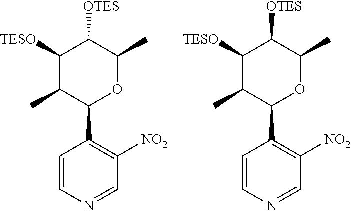

To a solution of the mixture of (+/−)-(2R,3S,4R,5S,6R)-2,5-dimethyl-6-(3-nitropyridin-4-yl)tetrahydro-2H-pyran-3,4-diol and (+/−)-(2R,3R,4R,5S,6R)-2,5-dimethyl-6-(3-nitropyridin-4-yl)tetrahydro-2H-pyran-3,4-diol (1 equiv.) and imidazole (7 equiv.) in DCM (0.2 M) was slowly added TESCl (5 equiv.) at 0° C. The reaction mixture was stirred for overnight and then quenched with water, diluted with EtOAc. The organic layer was washed with water and brine, dried over anhydrous sodium sulfate, filtered and concentrated in vacuo. The crude reaction products were purified by silica column chromatography to afford a mixture of (+/−)-4-((2R,3R,4R,5R,6R)-3,6-dimethyl-4,5-bis(triethylsilyloxy)tetrahydro-2H-pyran-2-yl)-3-nitropyridine and (+/−)-4-((2R,3R,4R,5S,6R)-3,6-dimethyl-4,5-bis(triethylsilyloxy)tetrahydro-2H-pyran-2-yl)-3-nitropyridine (75%). LCMS (m/z): 497.3 (MH⁺), Rₜ=0.64 min.

### Synthesis of (+/−)-4-((2R,3R,4R,5R,6R)-3,6-dimethyl-4,5-bis(triethylsilyloxy)tetrahydro-2H-pyran-2-yl)pyridin-3-amine and (+/−)-4-((2R,3R,4R,5S,6R)-3,6-dimethyl-4,5-bis(triethylsilyloxy)tetrahydro-2H-pyran-2-yl)pyridin-3-amine

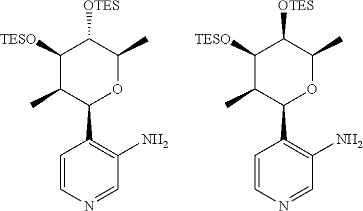

A mixture of (+/−)-4-((2R,3R,4R,5R,6R)-3,6-dimethyl-4,5-bis(triethylsilyloxy)tetrahydro-2H-pyran-2-yl)-3-nitropyridine and (+/−)-4-((2R,3R,4R,5S,6R)-3,6-dimethyl-4,5-bis(triethylsilyloxy)tetrahydro-2H-pyran-2-yl)-3-nitropyridine (1.0 equiv.) was dissolved in MeOH (0.1 M) and degassed with nitrogen for 15 min. Followed by addition of Pd(OH)₂ (0.2 equiv), the reaction mixture was placed under an H₂ balloon for 2 h. The mixture was filtered through Celite pad, washed with MeOH and EtOAc and concentrated in vacuo to afford a mixture of (+/−)-4-((2R,3R,4R,5R,6R)-3,6-dimethyl-4,5-bis(triethylsilyloxy)tetrahydro-2H-pyran-2-yl)pyridin-3-amine and (+/−)-4-((2R,3R,4R,5S,6R)-3,6-dimethyl-4,5-bis(triethylsilyloxy)tetrahydro-2H-pyran-2-yl)pyridin-3-amine (97%). LCMS (m/z): 467.5 (MH⁺), Rₜ=1.35 min. ¹H-NMR (400 MHz, CDCl₃): δ 7.93 (m, 4H), 6.93 (m, 1H), 6.91 (m, 1H), 4.59 (m, 1H), 4.56 (m, 1H), 4.29 (bs, 2H), 4.08 (bs, 2H), 3.77 (m, 2H), 3.65 (m, 1H), 3.55 (m, 1H), 3.41 (m, 1H), 3.34 (m, 1H), 2.25 (m, 1H), 1.98 (m, 1H), 1.34 (m, 3H), 1.28 (m, 3H), 0.99 (m, 30H), 0.84 (m, 3H), 0.67 (m, 24H), 0.59 (m, 3H).

### Synthesis of 4-(1,3-dioxolan-2-yl)-3-nitropyridine

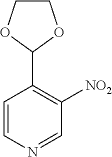 [SMILES: O=[N+]([O-])c1cnccc1C1OCCO1]

A solution of 3-nitroisonicotinaldehyde (1.0 equiv.), ethylene glycol (5.5 equiv.) and p-toluenesulfonic acid (0.10 equiv.) in toluene (0.15 M) was heated at reflux equipped with Dean Stark apparatus for 3 h. After cooling down, the reaction mixture was quenched with sat. NaHCO₃ solution, the reaction mixture was then extracted by EtOAc, the organic layer was washed by water and brine, dried over anhydrous sodium sulfate, filtered and concentrated in vacuo to afford 4-(1,3-dioxolan-2-yl)-3-nitropyridine in 78% yield. LCMS (m/z): 197.1 (MH⁺), Rₜ=0.51 min.

### Synthesis of 4-(1,3-dioxolan-2-yl)pyridin-3-amine

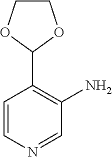 [SMILES: Nc1cnccc1C1OCCO1]

A solution of 4-(1,3-dioxolan-2-yl)-3-nitropyridine (1.0 equiv.) in methanol (0.3 M) was degassed by nitrogen for 10 min followed by addition of 10% Pd/C. The reaction mixture was stirred at room temperature for 5 h in a sealed steel vessel under hydrogen atmosphere at 50 psi. The reaction mixture was filtered through Celite pad and washed by MeOH and EtOAc. The filtrate was concentrated in vacuo to give 4-(1,3-dioxolan-2-yl)pyridin-3-amine in >99% yield. LCMS (m/z): 167.1 (MH⁺), Rₜ=0.24 min.

### Synthesis of 4-((2S,4S)-4-(benzyloxymethyl)-1,3-dioxolan-2-yl)-3-nitropyridine

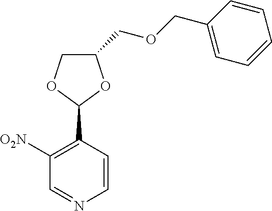 [SMILES: O=[N+]([O-])c1cnccc1[C@H]1OC[C@H](COCc2ccccc2)O1]

A solution of 3-nitroisonicotinaldehyde (1.0 equiv.), (R)-3-(benzyloxy) propane-1,2-diol (2 equiv.) and p-toluenesulfonic acid (0.10 equiv.) in toluene (0.15 M) was heated at reflux equipped with Dean Stark apparatus for 3 h. After cooling down, the reaction mixture was quenched with sat. NaHCO₃ solution, the reaction mixture was then extracted by EtOAc; the organic layer was washed by water and brine, dried over anhydrous sodium sulfate, filtered and concentrated in vacuo. The crude material was purified by silica gel column chromatography eluting with ethyl acetate and hexanes (1:2) to give 4-((2S,4S)-4-(benzyloxymethyl)-1,3-dioxolan-2-yl)-3-nitropyridine in 43% yield. LCMS (m/z): 317.0 (MH⁺), Rₜ=0.86 min.

### Synthesis of 4-((2S,4S)-4-(benzyloxymethyl)-1,3-dioxolan-2-yl)pyridin-3-amine

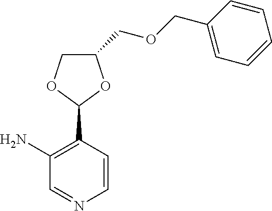 [SMILES: Nc1cnccc1[C@H]1OC[C@H](COCc2ccccc2)O1]

A solution of 4-((2S,4S)-4-(benzyloxymethyl)-1,3-dioxolan-2-yl)-3-nitropyridine (1.0 equiv.) in methanol (0.3 M) was degassed by nitrogen for 10 min, 10% Pd (OH)₂ (0.2 equiv) was added. The reaction mixture was stirred at room temperature for 1 h under hydrogen balloon. The reaction mixture was filtered through celite and washed by MeOH and EtOAc, the filtrate was concentrated in vacuo to give 4-((2S,4S)-4-(benzyloxymethyl)-1,3-dioxolan-2-yl)pyridin-3-amine in >99% yield. LCMS (m/z): 287.1 (MH⁺), Rₜ=0.59 min.

### Synthesis of 4-(1,3-dioxan-2-yl)-3-nitropyridine

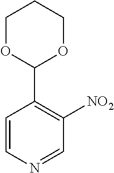 [SMILES: O=[N+]([O-])c1cnccc1C1OCCCO1]

A solution of 3-nitroisonicotinaldehyde (1 equiv.), 3-propanediol (3 equiv.), and p-toluenesulfonic acid (0.10 equiv.) in toluene (0.26 M) was heated at reflux equipped with Dean Stark apparatus for 3 h. After cooling down, the reaction mixture was quenched with sat. NaHCO₃ solution, the reaction mixture was then extracted by EtOAc, the organic layer was washed by water and brine, dried over anhydrous sodium sulfate, filtered and concentrated in vacuo to afford 4-(1,3-dioxan-2-yl)-3-nitropyridine in 78% yield. LCMS (m/z): 211.9 (MH⁺), Rₜ=0.71 min.

### Synthesis of 4-(1,3-dioxan-2-yl)pyridin-3-amine

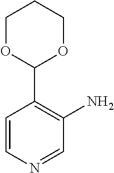 [SMILES: Nc1cnccc1C1OCCCO1]

A solution of 4-(1,3-dioxan-2-yl)-3-nitropyridine in Methanol (0.3 M) was degassed by nitrogen for 10 min followed by addition of 10% Pd/C. The reaction mixture was stirred at room temperature for 12 h in a sealed steel vessel under hydrogen atmosphere at 50 psi. The reaction mixture was filtered through Celite pad and washed by MeOH and EtOAc. The filtrate was concentrated in vacuo to afford 4-(1,3-dioxan-2-yl)pyridin-3-amine in 98% yield. LCMS (m/z): 181.0 (MH⁺), Rₜ=0.28 min

### Synthesis of trans/cis (2-(3-nitropyridin-4-yl)-1,3-dioxan-5-yl)methanol

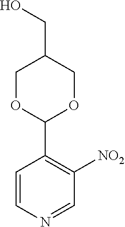 [SMILES: O=[N+]([O-])c1cnccc1C1OCC(CO)CO1]

A solution of 3-nitroisonicotinaldehyde (1.0 equiv.), 2-(hydroxymethyl) propane-1,3-diol (2.3 equiv.) and p-toluenesulfonic acid (0.10 equiv.) in toluene (0.5 M) was heated at reflux equipped with Dean Stark apparatus for 12 h. After cooling down, the reaction mixture was quenched with sat. NaHCO₃ solution, the reaction mixture was then extracted by EtOAc, the organic layer was washed by water and brine, dried over anhydrous sodium sulfate, filtered and concentrated in vacuo to afford (2-(3-nitropyridin-4-yl)-1,3-dioxan-5-yl)methanol in 86% yield. LCMS (m/z): 241.0 (MH⁺), Rₜ=0.46 min.

### Synthesis of trans/cis(2-(3-nitropyridin-4-yl)-1,3-dioxan-5-yl)methyl acetate

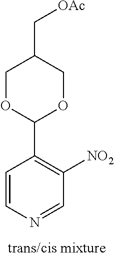 [SMILES: CC(=O)OCC1COC(c2ccncc2[N+](=O)[O-])OC1]

A solution of (2-(3-nitropyridin-4-yl)-1,3-dioxan-5-yl)methanol (1.0 equiv.) in pyridine (0.5 M), was added acetic anhydride (1.5 equiv.), the reaction mixture was stirred at room temperature for 12 h, After quenched by NaHCO₃, the reaction mixture was extracted by EtOAc, the organic washed with water and brine, and dried with anhydrous sodium sulfate, filtered, and concentrated in vacuo. The crude material was purified by silica gel column chromatography eluting with ethyl acetate and hexanes to give trans/cis (2-(3-nitropyridin-4-yl)-1,3-dioxan-5-yl)methyl acetate in 100% yield. LCMS (m/z): 283.0 (MH⁺), Rₜ=0.71 min.

### Synthesis of trans/cis (2-(3-aminopyridin-4-yl)-1,3-dioxan-5-yl)methyl acetate

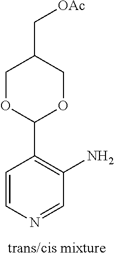 [SMILES: CC(=O)OCC1COC(c2ccncc2N)OC1]

A solution of trans/cis(2-(3-nitropyridin-4-yl)-1,3-dioxan-5-yl)methyl acetate (1.0 equiv.) in methanol (0.3 M) was degassed by nitrogen for 10 min, 20% Pd(OH)₂ (0.5 equiv) was added, the reaction mixture was stirred at room temperature under hydrogen balloon for 12 h. The reaction mixture was filtered through Celite pad and washed by MeOH and EtOAc. The filtrate was concentrated in vacuo to give trans/cis (2-(3-aminopyridin-4-yl)-1,3-dioxan-5-yl)methyl acetate in 58% yield. LCMS (m/z): 253.1 (MH⁺), Rₜ=0.38 min

### Synthesis of trans/cis (2-(3-(6-(2,6-difluorophenyl)-5-fluoropicolinamido)pyridin-4-yl)-1,3-dioxan-5-yl)methyl acetate

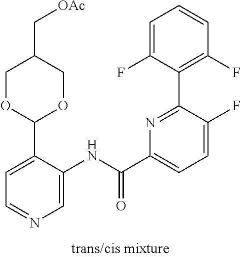 [SMILES: CC(=O)OCC1COC(c2ccncc2NC(=O)c2ccc(F)c(-c3c(F)cccc3F)n2)OC1]

A solution of trans/cis (2-(3-aminopyridin-4-yl)-1,3-dioxan-5-yl)methyl acetate (1.0 equiv.) and 6-(2,6-difluorophenyl)-5-fluoropicolinic acid (1.1 equiv.), HOAT (1.2 equiv.) and EDC(1.2 equiv.) in DMF (0.5 M) was stirred for 12 h at room temperature. The reaction mixture was partitioned between EtOAc and NaHCO₃, the organic was washed by water and brine, dried over anhydrous sodium sulfate, filtered and concentrated in vacuo to give trans/cis (2-(3-(6-(2,6-difluorophenyl)-5-fluoropicolinamido)pyridin-4-yl)-1,3-dioxan-5-yl)methyl acetate in 66% yield. LCMS (m/z): 488.2 (MH⁺), Rₜ=0.76 min.

### Synthesis of Trans/Cis 6-(2,6-difluorophenyl)-5-fluoro-N-(4-(5-(hydroxymethyl)-1,3-dioxan-2-yl)pyridin-3-yl)picolinamide

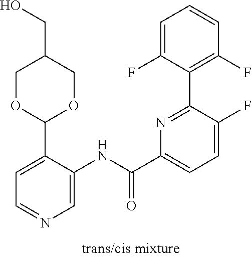 [SMILES: O=C(Nc1cnccc1C1OCC(CO)CO1)c1ccc(F)c(-c2c(F)cccc2F)n1]

A solution of trans/cis (2-(3-(6-(2,6-difluorophenyl)-5-fluoropicolinamido) pyridin-4-yl)-1,3-dioxan-5-yl)methyl acetate (1.0 equiv.) in methanol/THF (1:2, 0.2 M) was added 1 N LiOH (2 equiv.), the reaction mixture was stirred at room temperature for 3 h. After neutralized with 1 N HCl solution, the reaction mixture was extracted by EtOAc, the organic phase was washed by water and brine, dried over anhydrous sodium sulfate, filtered and concentrated in vacuo to give trans/cis 6-(2,6-difluorophenyl)-5-fluoro-N-(4-(5-(hydroxymethyl)-1,3-dioxan-2-yl)pyridin-3-yl)picolinamidein in 100% yield. LCMS (m/z): 467.2 (MH⁺), Rₜ=0.70 min.

### Synthesis of Trans N-(4-(5-((tert-butyldimethylsilyloxy)methyl)-1,3-dioxan-2-yl)pyridin-3-yl)-6-(2,6-difluorophenyl)-5-fluoropicolinamide and Cis N-(4-(5-((tert-butyldimethylsilyloxy)methyl)-1,3-dioxan-2-yl)pyridin-3-yl)-6-(2,6-difluorophenyl)-5-fluoropicolinamide

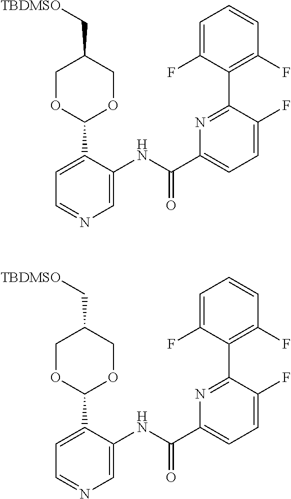 [SMILES: CC(C)(C)[Si](C)(C)OC[C@H]1CO[C@H](c2ccncc2NC(=O)c2ccc(F)c(-c3c(F)cccc3F)n2)OC1 ; CC(C)(C)[Si](C)(C)OC[C@H]1CO[C@@H](c2ccncc2NC(=O)c2ccc(F)c(-c3c(F)cccc3F)n2)OC1]

To a solution of trans/cis 6-(2,6-difluorophenyl)-5-fluoro-N-(4-(5-(hydroxymethyl)-1,3-dioxan-2-yl)pyridin-3-yl) picolinamide (1.0 equiv.) in DCM (0.3 M) was added imidazole (1.3 equiv.), TBDMSCl (1.1 equiv.) at room temperature. The reaction mixture was stirred at room temperature for 2 h. After quenched with NaHCO₃, the reaction mixture was extracted with EtOAc. The combined organic layer was washed with water and brine, dried over anhydrous sodium sulfate. After filtered and concentrated in vacuo, the crude material was purified by reverse-phase HPLC to yield two diastereomers (relative stereochemistry was assigned arbitrarily): trans N-(4-(5-((tert-butyldimethylsilyloxy)methyl)-1,3-dioxan-2-yl)pyridin-3-yl)-6-(2,6-difluorophenyl)-5-fluoropicolinamide: LCMS (m/z): 560.2 (MH⁺), Rt=1.11 min and cis N-(4-(−5-((tert-butyldimethylsilyloxy)methyl)-1,3-dioxan-2-yl)pyridin-3-yl)-6-(2,6-difluorophenyl)-5-fluoropicolinamide. LCMS (m/z): 560.2 (MH⁺), Rₜ=1.14 min.

### Synthesis of trans 6-(2,6-difluorophenyl)-5-fluoro-N-(4-(5-(hydroxymethyl)-1,3-dioxan-2-yl)pyridin-3-yl)picolinamide

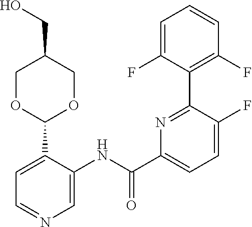 [SMILES: O=C(Nc1cnccc1[C@H]1OC[C@H](CO)CO1)c1ccc(F)c(-c2c(F)cccc2F)n1]

A solution of trans N-(4-(5-((tert-butyldimethylsilyloxy)methyl)-1,3-dioxan-2-yl)pyridin-3-yl)-6-(2,6-difluorophenyl)-5-fluoropicolinamide in THF (0.1 M) was added TBAF (1.0 equiv.). The reaction mixture was stirred at room temperature for 3 h. After worked up with EtOAc, the crude product was purified by reverse-phase prep HPLC. The HPLC fractions was added to EtOAc and solid Na₂CO₃, separated and washed with brine Upon drying over sodium sulfate, filtering and removing the volatiles in vacuo the free base of trans 6-(2,6-difluorophenyl)-5-fluoro-N-(4-(5-(hydroxymethyl)-1,3-dioxan-2-yl)pyridin-3-yl) picolinamide was obtained. LCMS (m/z): 446.1 (MH⁺), Rₜ=0.67 min.

### Synthesis of cis 6-(2,6-difluorophenyl)-5-fluoro-N-(4-(5-(hydroxymethyl)-1,3-dioxan-2-yl)pyridin-3-yl)picolinamide

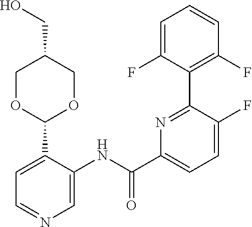 [SMILES: O=C(Nc1cnccc1[C@H]1OC[C@@H](CO)CO1)c1ccc(F)c(-c2c(F)cccc2F)n1]

A solution of cis N-(4-(5-((tert-butyldimethylsilyloxy)methyl)-1,3-dioxan-2-yl)pyridin-3-yl)-6-(2,6-difluorophenyl)-5-fluoropicolinamide (1.0 equiv.) in THF (0.1 M) was added TBAF (1.0 equiv.). The reaction mixture was stirred at room temperature for 3 h. After worked up with EtOAc, the crude product was purified by reverse-phase prep HPLC. The HPLC fractions was added to EtOAc and solid Na₂CO₃, separated and washed with brine. Upon drying over sodium sulfate, filtering and removing the volatiles in vacuo the free base of cis 6-(2,6-difluorophenyl)-5-fluoro-N-(4-(5-(hydroxymethyl)-1,3-dioxan-2-yl)pyridin-3-yl)picolinamide was obtained. LCMS (m/z): 446.0 (MH⁺), Rₜ=0.65 min.

### Synthesis of ((2R,4R)-2-(3-nitropyridin-4-yl)-1,3-dioxan-4-yl)methanol

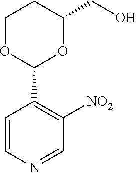 [SMILES: O=[N+]([O-])c1cnccc1[C@@H]1OCC[C@H](CO)O1]

A solution of 3-nitroisonicotinaldehyde (1 equiv.), (R)-butane-1,2,4-triol (4 equiv.) and p-toluenesulfonic acid (0.10 equiv.) in toluene (0.05 M) was heated at reflux equipped with Dean Stark apparatus for 12 h. After cooling down, the reaction mixture was quenched with sat. NaHCO₃ solution, the reaction mixture was then extracted by EtOAc, the organic layer was washed by water and brine, dried over anhydrous sodium sulfate, filtered and concentrated in vacuo to afford ((2R,4R)-2-(3-nitropyridin-4-yl)-1,3-dioxan-4-yl)methanol in 95% yield. LCMS (m/z): 241.0 (MH⁺), Rₜ=0.50 min.

### Synthesis of 4-((2R,4R)-4-((tert-butyldimethylsilyloxy)methyl)-1,3-dioxan-2-yl)-3-nitropyridine

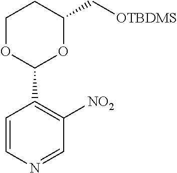 [SMILES: CC(C)(C)[Si](C)(C)OC[C@H]1CCO[C@@H](c2ccncc2[N+](=O)[O-])O1]

To a solution of ((2R,4R)-2-(3-nitropyridin-4-yl)-1,3-dioxan-4-yl)methanol (1 equiv.) in DCM (0.5 M) was added Imidazole (2 equiv.), TBDMSCl (1.5 equiv.) at room temperature. The reaction mixture was stirred at room temperature for 12 h. After quenched with NaHCO₃, the reaction mixture was extracted with EtOAc. The combined organic layer was washed with water and brine, dried over anhydrous sodium sulfate. Filtered and concentrated in vacuo. The crude material was purified by silica gel column chromatography eluting with ethyl acetate and hexanes to give 4-((2R,4R)-4-((tert-butyldimethylsilyloxy)methyl)-1,3-dioxan-2-yl)-3-nitropyridine in 40% yield. LCMS (m/z): 355.1.0 (MH⁺), Rₜ=1.29 min.

### Synthesis of 4-((2R,4R)-4-((tert-butyldimethylsilyloxy)methyl)-1,3-dioxan-2-yl) pyridin-3-amine

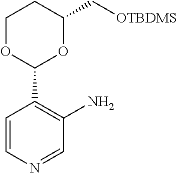 [SMILES: CC(C)(C)[Si](C)(C)OC[C@H]1CCO[C@@H](c2ccncc2N)O1]

A solution of 4-((2R,4R)-4-((tert-butyldimethylsilyloxy)methyl)-1,3-dioxan-2-yl)-3-nitropyridine (1.0 equiv.) in methanol (0.1 M) was degassed by nitrogen for 10 min, 20% Pd(OH)₂ (0.5 equiv) was added, the reaction mixture was stirred at room temperature under hydrogen balloon for 12 h. The reaction mixture was filtered through Celite pad and washed by MeOH and EtOAc. The filtrate was concentrated in vacuo to give 4-((2R,4R)-4-((tert-butyldimethylsilyloxy)methyl)-1,3-dioxan-2-yl)pyridin-3-amine in 80% yield. LCMS (m/z): 325.1 (MH⁺), Rₜ=0.84 min.

### Synthesis of N-(4-((2R,4R,5R,6R)-4,5-bis(triisopropylsilyloxy)-6-((triisopropylsilyloxy)methyl)tetrahydro-2H-pyran-2-yl)pyridin-3-yl)-6-(2,6-difluorophenyl)-5-fluoropicolinamide

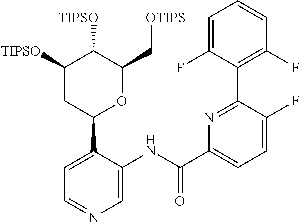

A solution of 4-((2R,4R,5R,6R)-4,5-bis(triisopropylsilyloxy)-6-((triisopropylsilyloxy)methyl)tetrahydro-2H-pyran-2-yl)pyridin-3-amine (1.0 equiv.) and 6-(2,6-difluorophenyl)-5-fluoropicolinic acid (1.1 equiv.), HOAT (1.2 equiv.) and EDC (1.2 equiv.) in DMF (0.5 M) was stirred for 12 hours at room temperature. The reaction mixture was partitioned between EtOAc and NaHCO₃; the organic layer was washed by water and brine, dried over anhydrous sodium sulfate, filtered and concentrated in vacuo. The crude material was purified by silica gel column chromatography eluting with ethyl acetate and hexanes (1:5) to give N-(4-((2R,4R,5R,6R)-4,5-bis(triisopropylsilyloxy)-6-((triisopropylsilyloxy)methyl)tetrahydro-2H-pyran-2-yl)pyridin-3-yl)-6-(2,6-difluorophenyl)-5-fluoropicolinamidein 50% yield. LCMS (m/z): 944.4 (MH⁺), Rₜ=0.95 min. (95/95B-Highmass).

### Synthesis of 6-(2,6-difluorophenyl)-5-fluoro-N-(4-((2R,4R,5R,6R)-6-(hydroxymethyl)-4,5-bis(triisopropylsilyloxy)tetrahydro-2H-pyran-2-yl)pyridin-3-yl)picolinamide

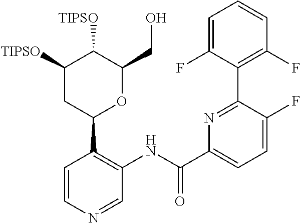

To a solution of N-(4-((2R,4R,5R,6R)-4,5-bis(triisopropylsilyloxy)-6-((triisopropylsilyloxy)methyl)tetrahydro-2H-pyran-2-yl)pyridin-3-yl)-6-(2,6-difluorophenyl)-5-fluoropicolinamide in THF (0.1 M) was added HCl(conc) (10 equiv) at room temperature. The reaction mixture was stirred at room temperature for 1.5 h. 3N NaOH solution was added to PH=12, the reaction mixture was extracted with EtOAc 3 times. The combined organic layer was washed with water and brine, dried over anhydrous sodium sulfate. Filtered and concentrated in vacuo. The crude material was purified by silica gel column chromatography eluting with ethyl acetate and hexanes (2:3) to give 6-(2,6-difluorophenyl)-5-fluoro-N-(4-((2R,4R,5R,6R)-6-(hydroxymethyl)-4,5-bis(triisopropylsilyloxy)tetrahydro-2H-pyran-2-yl)pyridin-3-yl)picolinamide in 50% yield. LC/MS (m/z): 788.7 (MH⁺), Rₜ=1.04 min (65-95% B).

### Synthesis of 6-(2,6-difluorophenyl)-5-fluoro-N-(4-((2R,4R,5R,6S)-6-(fluoromethyl)-4,5-bis(triisopropylsilyloxy)tetrahydro-2H-pyran-2-yl)pyridin-3-yl)picolinamide

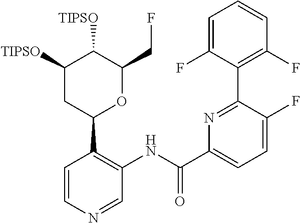

To a solution of 6-(2,6-difluorophenyl)-5-fluoro-N-(4-((2R,4R,5R,6R)-6-(hydroxymethyl)-4,5-bis(triisopropylsilyloxy)tetrahydro-2H-pyran-2-yl)pyridin-3-yl)picolinamide in Dichloromethane (0.3M) was added DAST (1.1 equiv.) at 0° C. The reaction mixture was stirred at room temperature for overnight. After quenching with sat. NaHCO₃ solution, the reaction mixture was extracted with Dichloromethane 3 times. The combined organic layer was washed with water and brine, dried over anhydrous sodium sulfate, filtered and concentrated in vacuo. The crude material was purified by silica gel column chromatography eluting with ethyl acetate and hexanes (2:3) to give 6-(2,6-difluorophenyl)-5-fluoro-N-(4-((2R,4R,5R,6S)-6-(fluoromethyl)-4,5-bis(triisopropylsilyloxy)tetrahydro-2H-pyran-2-yl)pyridin-3-yl)picolinamide in 13% yield. LC/MS (m/z): 790.8 (MH⁺), Rₜ=1.24 min, (65-95B)

### Synthesis of 6-(2,6-difluorophenyl)-5-fluoro-N-(4-((2R,4R,5S,6S)-6-(fluoromethyl)-4,5-dihydroxytetrahydro-2H-pyran-2-yl)pyridin-3-yl) picolinamide

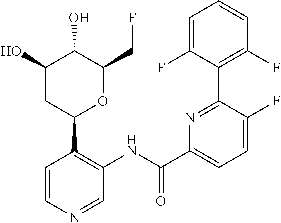 [SMILES: O=C(Nc1cnccc1[C@H]1C[C@@H](O)[C@H](O)[C@@H](CF)O1)c1ccc(F)c(-c2c(F)cccc2F)n1]

To a solution of 6-(2,6-difluorophenyl)-5-fluoro-N-(4-((2R,4R,5R,6S)-6-(fluoromethyl)-4,5-bis(triisopropylsilyloxy)tetrahydro-2H-pyran-2-yl)pyridin-3-yl)picolinamide in THF (0.3 M) was added TBAF (1.0 equiv.) at room temperature. The reaction mixture was stirred at room temperature for 12 h. The reaction mixture was diluted with EtOAc and NaHCO₃ solution. The organic layer was washed with water and brine, dried over anhydrous sodium sulfate. Filtered and concentrated in vacuo. The crude product was purified by reverse-phase HPLC and the pure fraction were lyophilized to give the 6-(2,6-difluorophenyl)-5-fluoro-N-(4-((2R,4R,5S,6S)-6-(fluoromethyl)-4,5-dihydroxytetrahydro-2H-pyran-2-yl)pyridin-3-yl) picolinamide as TFA salt. LC/MS (m/z): 478.1 (MH⁺), Rₜ=0.62 min,

### Synthesis of (±)(2R,6R)-3-((dimethylamino) methyl)-2-methyl-6-(3-nitropyridin-4-yl)dihydro-2H-pyran-4(3H)-one

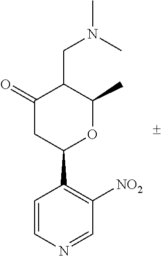 [SMILES: C[C@H]1O[C@@H](c2ccncc2[N+](=O)[O-])CC(=O)C1CN(C)C]

To a solution of N-methyl-N-methylenemethanaminium iodide (2 equiv.) in DCM (0.4 M) was added (±) 4-((2R,6R)-6-methyl-4-(triethylsilyloxy)-3,6-dihydro-2H-pyran-2-yl)-3-nitropyridine in dichloromethane at room temperature the reaction mixture was stirred for 3 days. Aqueous 1N HCl (2 equiv.) was added into the reaction mixture, and after stirring at room temperature for 1 h, the reaction mixture was basify to PH=12 by addition of 3 N NaOH solution. The reaction mixture was then extracted by EtOAc, the organic was washed by water and brine, dried over anhydrous sodium sulfate, filtered and concentrated in vacuo to give (±)(2R,6R)-3-((dimethylamino) methyl)-2-methyl-6-(3-nitropyridin-4-yl) dihydro-2H-pyran-4(3H)-one in 100% yield. LCMS (m/z): 294.1 (MH⁺), Rₜ=0.41 min.

### Synthesis of (±)(2R,6R)-2-methyl-3-methylene-6-(3-nitropyridin-4-yl)dihydro-2H-pyran-4(3H)-one

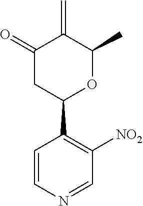 [SMILES: C=C1C(=O)C[C@H](c2ccncc2[N+](=O)[O-])O[C@@H]1C]

To a solution of (±)(2R,6R)-3-((dimethylamino)methyl)-2-methyl-6-(3-nitropyridin-4-yl)dihydro-2H-pyran-4(3H)-one crude in THF (0.5 M) was added MeI (2 equiv) at 0° C. The reaction mixture was allowed to warm up to room temperature and stirred at room temperature for 48 h. Sat. NaHCO₃ was added, the reaction mixture was stirred at room temperature for 30 minutes, some THF was removed in vacuo. The reaction mixture was extracted with EtOAc 3 times. The combined organic layer was washed with water and brine, dried over anhydrous sodium sulfate. Filtered and concentrated in vacuo. The crude material was purified by silica gel column chromatography eluting with ethyl acetate and hexanes (1:4) to afford (±)(2R,6R)-2-methyl-3-methylene-6-(3-nitropyridin-4-yl)dihydro-2H-pyran-4(3H)-one in 10% yield. LC/MS (m/z): 249.0 (MH⁺), Rₜ=0.68 min.

### Synthesis of (±)(2R,4R,6R)-2-methyl-3-methylene-6-(3-nitropyridin-4-yl)tetrahydro-2H-pyran-4-ol

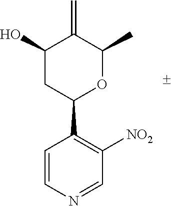 [SMILES: C=C1[C@H](O)C[C@H](c2ccncc2[N+](=O)[O-])O[C@@H]1C]

To a solution of (±)(2R,6R)-2-methyl-3-methylene-6-(3-nitropyridin-4-yl)dihydro-2H-pyran-4(3H)-one in methanol (0.2M) was added cerium(III) chloride heptahydrate (1.1 equiv) at room temperature. The reaction mixture was stirred at room temperature for 1 h, then cooled down to 0° C. NaBH₄ (1.1 equiv) was added slowly. The reaction mixture was allowed to warm to room temperature and stirred at room temperature for 1 h. After quenched with H₂O, The reaction mixture was extracted with EtOAc 3 times. The combined organic layer was washed with water and brine, dried over anhydrous sodium sulfate. Filtered and concentrated in vacuo to give (±)(2R,4R,6R)-2-methyl-3-methylene-6-(3-nitropyridin-4-yl)tetrahydro-2H-pyran-4-ol in 94% yield. LC/MS (m/z): 251.1 (MH⁺), Rₜ=0.61 min.

### Synthesis of (±)(2R,3S,4R,6R)-3-(hydroxymethyl)-2-methyl-6-(3-nitropyridin-4-yl)tetrahydro-2H-pyran-3,4-diol

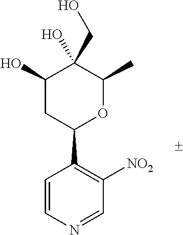 [SMILES: C[C@H]1O[C@@H](c2ccncc2[N+](=O)[O-])C[C@@H](O)[C@@]1(O)CO]

To a solution of (±)(2R,4R,6R)-2-methyl-3-methylene-6-(3-nitropyridin-4-yl)tetrahydro-2H-pyran-4-ol in Acetone/H₂O (4:1, 0.05 M) was added osmium tetroxide (4% in H₂O) (0.04 equiv.) and N-methylmorpholine oxide (2 equiv.) at room temperature. The reaction mixture was stirred at room temperature for 12 h. After quenching with Sodium thiosulfate and NaHCO₃, the reaction mixture was extracted with EtOAc 3 times. The combined organic layer was washed with water and brine, dried over anhydrous sodium sulfate. Filtered and concentrated in vacuo to yield (±)(2R,3S,4R,6R)-3-(hydroxymethyl)-2-methyl-6-(3-nitropyridin-4-yl)tetrahydro-2H-pyran-3,4-diol was used in next step reaction. LC/MS (m/z): 285.0 (MH⁺), Rₜ=0.41 min.

### Synthesis of (±)(2R,3R,4R,6R)-4-(tert-butyldimethylsilyloxy)-3-((tert-butyldimethylsilyloxy)methyl)-2-methyl-6-(3-nitropyridin-4-yl)tetrahydro-2H-pyran-3-ol

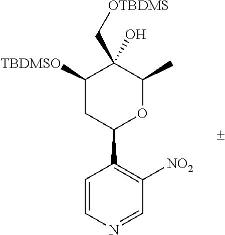 [SMILES: C[C@H]1O[C@@H](c2ccncc2[N+](=O)[O-])C[C@@H](O[Si](C)(C)C(C)(C)C)[C@@]1(O)CO[Si](C)(C)C(C)(C)C]

To a solution of (±)(2R,3S,4R,6R)-3-(hydroxymethyl)-2-methyl-6-(3-nitropyridin-4-yl)tetrahydro-2H-pyran-3,4-diol (1.0 equiv.) in DMF (0.5M) was added imidazole (5 equiv.), TBDMS-Cl (3.5 equiv.) at room temperature. The reaction mixture was stirred at room temperature for 12 h. After quenching with NaHCO₃, the reaction mixture was extracted with EtOAc 3 times. The combined organic layer was washed with water and brine, dried over anhydrous sodium sulfate. Filtered and concentrated in vacuo. The crude material was purified by silica gel column chromatography eluting with ethyl acetate and hexanes (1:2) to afford (±)(2R,3R,4R,6R)-4-(tert-butyldimethylsilyloxy)-3-((tert-butyldimethylsilyloxy)methyl)-2-methyl-6-(3-nitropyridin-4-yl)tetrahydro-2H-pyran-3-ol in 57% yield. LC/MS (m/z): 513.2 (MH⁺), Rₜ=0.49 min (95/95 method).

### Synthesis of (±)(2R,3R,4R,6R)-6-(3-aminopyridin-4-yl)-4-(tert-butyldimethylsilyloxy)-3-((tert-butyldimethylsilyloxy)methyl)-2-methyltetrahydro-2H-pyran-3-ol

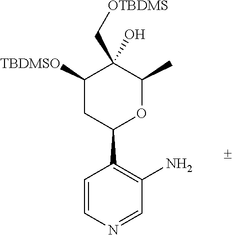 [SMILES: C[C@H]1O[C@@H](c2ccncc2N)C[C@@H](O[Si](C)(C)C(C)(C)C)[C@@]1(O)CO[Si](C)(C)C(C)(C)C]

A solution of (±)(2R,3R,4R,6R)-4-(tert-butyldimethylsilyloxy)-3-((tert-butyldimethylsilyloxy)methyl)-2-methyl-6-(3-nitropyridin-4-yl)tetrahydro-2H-pyran-3-ol (1.0 equiv.) in methanol (0.3M) was degassed with nitrogen for 10 min, then added 10% Pd/C (0.1 equiv). The reaction mixture was stirred at room temperature under a hydrogen balloon for 1 h. The reaction mixture was filtered through celite and concentrated to afford (±)-(2R,3R,4R,6R)-6-(3-aminopyridin-4-yl)-4-(tert-butyldimethylsilyloxy)-3-((tert-butyldimethylsilyloxy)methyl)-2-methyltetrahydro-2H-pyran-3-ol in 99% yield. LC/MS (m/z): 483.4 (MH⁺), Rₜ=0.23 min. (±)(2R,3R,4R,6R)-6-(3-aminopyridin-4-yl)-4-(tert-butyldimethylsilyloxy)-3-((tert-butyldimethylsilyloxy)methyl)-2-methyltetrahydro-2H-pyran-3-ol was subjected to chiral separation to afford two enantiomer, (2S,3S,4S,6S)-6-(3-aminopyridin-4-yl)-4-(tert-butyldimethylsilyloxy)-3-((tert-butyldimethylsilyloxy)methyl)-2-methyltetrahydro-2H-pyran-3-ol Rₜ=8.90 min (IC column. 1 mL/min, heptane/IPA=95/5,); (2R,3R,4R,6R)-6-(3-aminopyridin-4-yl)-4-(tert-butyldimethylsilyloxy)-3-((tert-butyldimethylsilyloxy)methyl)-2-methyltetrahydro-2H-pyran-3-ol Rₜ=10.59 min (IC column, 1 mL/min, heptane/IPA=95/5).

### Synthesis of 4-iodo-3-nitropyridine

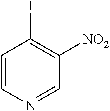

To a solution of 4-chloro-3-nitropyridine (1.0 equiv.) in ACN (0.118 M) was added sodium iodide (18.0 equiv.). The mixture was stirred for 30 min. under N₂. Sat. sodium bicarbonate was added and the mixture extracted with EtOAc. The combined organics were washed with 10% Na₂S₂O₃, brine, dried over sodium sulfate, filtered and concentrated to give 4-iodo-3-nitropyridine in 87% yield. LC/MS (m/z): 250.9 (MH⁺), Rₜ=0.62 min. ¹H NMR (400 MHz, CHLOROFORM-d) δ ppm 8.03 (d, 1H) 8.35 (d, 1H) 9.03 (s, 1H).

### Synthesis of 4-(3,4-dihydro-2H-pyran-6-yl)-3-nitropyridine

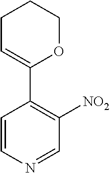 [SMILES: O=[N+]([O-])c1cnccc1C1=CCCCO1]

[2-(5,6-Dihydro-4H-pyranyl)]dimethylsilanol (1.2 equiv.) was dissolved in TBAF (1.0 M in THF) (2.0 equiv) and stirred for 10 min. 4-iodo-3-nitropyridine (1.0 equiv.) and [allylPdCl]2 (0.025 equiv.) were added. The suspension was stirred for 20 min. and then [2-(5,6-Dihydro-4H-pyranyl)]dimethylsilanol (2.0 equiv.), TBAF (1.0 M in THF) (2.0 equiv.) and [allylPdCl]2 (0.025 equiv.) were added and the reaction stirred for 1.5 hours. The reaction mixture was loaded onto a RediSep column and purified by ISCO eluting with 0-100% EtOAc in Heptanes to give 4-(3,4-dihydro-2H-pyran-6-yl)-3-nitropyridine in 43.6% yield. LC/MS (m/z): 207.0 (MH⁺), Rₜ=0.73 min. ¹H NMR (400 MHz, CHLOROFORM-d) δ ppm 1.93 (m, 2H) 2.22-2.30 (m, 2H) 4.04-4.10 (m, 2H) 5.39 (t, 1H) 7.40 (d, 1H) 8.71 (d, 1H) 8.90 (s, 1H).

### Synthesis of (+/−)4-(tetrahydro-2H-pyran-2-yl)pyridin-3-amine

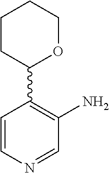 [SMILES: Nc1cnccc1C1CCCCO1]

4-(3,4-dihydro-2H-pyran-6-yl)-3-nitropyridine (1.0 equiv.) was dissolved in MeOH (0.2 M) and degassed with vacuum to Argon. Pd/C (10% degussa type 101 NE/W) (0.5 equiv.) was added and the mixture was stirred under a balloon of H₂ for 4 hours. The mixture was passed through a 1.0 uM PTFE ACRODISC CR filter and evaporated in vacuo to give 4-(tetrahydro-2H-pyran-2-yl)pyridin-3-amine in 71% yield. LC/MS (m/z): 179.2 (MH⁺) Rₜ=0.40 min.

### Synthesis of Cis (+/−)4-(6-methyl-4-(triethylsilyloxy)-3,6-dihydro-2H-pyran-2-yl)-3-nitropyridine

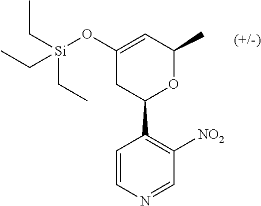 [SMILES: CC[Si](CC)(CC)OC1=C[C@@H](C)O[C@@H](c2ccncc2[N+](=O)[O-])C1]

Triethyl(penta-1,3-dien-2-yloxy)silane (2.7 equiv.), 3-nitroisonicotinaldehyde (1.0 equiv.) and tris(6,6,7,7,8,8,8-heptafluoro-2,2-dimethyl-3,5-octanedionato) europium (0.05 equiv.) were dissolved in CHCl₃ (1.315 M) in a flame dried rbf and stirred at 60° C. under Argon for 45 min. The heat was turned off and the reaction stirred 16 hours at room temperature. The volatiles were removed in vacuo and the liquid was loaded on to a RediSep column and purified by ISCO eluting with 0-30% EtOAc in Heptanes to give Cis (+/−)4-(6-methyl-4-(triethylsilyloxy)-3,6-dihydro-2H-pyran-2-yl)-3-nitropyridine in 84% yield. LC/MS (m/z): 351.1 (MH⁺) Rₜ=1.33 min. ¹H NMR (400 MHz, CHLOROFORM-d) δ ppm 0.70 (m, 6H) 1.00 (t, 9H) 1.30 (d, 3H) 2.18 (m, 1H) 2.48 (m, 1H) 4.38-4.45 (m, 1H) 4.87 (s, 1H) 5.26 (dd, 1H) 7.84 (d, 1H) 8.84 (d, 1H) 9.17 (s, 1H).

### Synthesis of Cis (+/−)2-methyl-6-(3-nitropyridin-4-yl)dihydro-2H-pyran-4(3H)-one

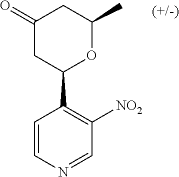 [SMILES: C[C@@H]1CC(=O)C[C@H](c2ccncc2[N+](=O)[O-])O1]

To a solution of Cis (+/−)4-(6-methyl-4-(triethylsilyloxy)-3,6-dihydro-2H-pyran-2-yl)-3-nitropyridine (1.0 equiv.) in THF (0.2 M) was added HCl (1.0 M) (1.16 equiv.). The reaction was stirred for 1 hour. NaOH (1.0 M) (1.16 equiv.) was added and the volatiles removed in vacuo. The residue was dissolved in EtOAc and washed with sat. sodium bicarbonate, brine, dried over sodium sulfate, filtered and concentrated to give Cis (+/−)2-methyl-6-(3-nitropyridin-4-yl)dihydro-2H-pyran-4(3H)-one in 80% yield. LC/MS (m/z): 237.0 (MH⁺), Rₜ=0.60 min. ¹H NMR (400 MHz, CHLOROFORM-d) δ ppm 1.42 (d, 3H) 2.30-2.43 (m, 2H) 2.52-2.59 (m, 1H) 2.87-2.94 (m, 1H) 3.94-4.04 (m, 1H) 5.35 (dd, 1H) 7.86 (d, 1H) 8.88 (d, 1H) 9.21 (s, 1H).

### Synthesis of (+/−)N-benzyl-2-methyl-6-(3-nitropyridin-4-yl)tetrahydro-2H-pyran-4-amine

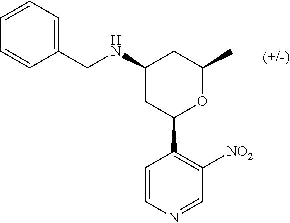 [SMILES: C[C@@H]1C[C@H](NCc2ccccc2)C[C@H](c2ccncc2[N+](=O)[O-])O1]

(+/−)2-methyl-6-(3-nitropyridin-4-yl)dihydro-2H-pyran-4(3H)-one (1.0 equiv.) was dissolved in MeOH (0.2 M) under N₂ and phenylmethanamine (2.0 equiv.) was added. The reaction was stirred for 2 hours. The reaction was cooled to −78° C. and lithium tetrahydroborate (2.0 M in THF) (1.1 equiv.) was added drop wise. The cooling bath was removed and the reaction stirred for 2 hours allowing to warm to room temperature. The solution was diluted with EtOAc and washed with sat. sodium bicarbonate, brine, dried over sodium sulfate, filtered and concentrated in vacuo to give (+/−)N-benzyl-2-methyl-6-(3-nitropyridin-4-yl)tetrahydro-2H-pyran-4-amine in 82% yield. LC/MS (m/z): 328.1 (MH⁺), Rₜ=0.59 min.

### Synthesis of (+/−)N-benzyl-2-methyl-6-(3-nitropyridin-4-yl)tetrahydro-2H-pyran-4-amine

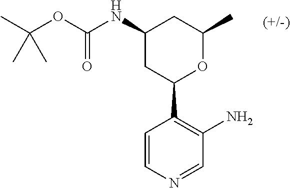 [SMILES: C[C@@H]1C[C@H](NC(=O)OC(C)(C)C)C[C@H](c2ccncc2N)O1]

(+/−)N-benzyl-2-methyl-6-(3-nitropyridin-4-yl)tetrahydro-2H-pyran-4-amine (1.0 equiv.) was dissolved in MeOH (0.2 M) and degassed with vacuum to Ar. Palladium hydroxide (0.2 equiv.) was added and the mixture placed under a H₂ balloon for 20 hours. Di-tert-butyl dicarbonate (1.8 equiv.) was added and the reaction stirred for 2 hours. The mixture was filtered through a 1 uM PTFE ACRODISC CR filter and concentrated. The residue was purified by ISCO with a Redisep column eluting with 0-100% (10% MeOH in DCM) in DCM to give tert-butyl (+/−)2-(3-aminopyridin-4-yl)-6-methyltetrahydro-2H-pyran-4-ylcarbamate in 45% yield. LC/MS (m/z): 328.1 (MH⁺), Rₜ=0.61 min. The material was separated via chiral HPLC (AD-H column, heptane:EtOH 90:10) to give tert-butyl (2S,4R,6S)-2-(3-aminopyridin-4-yl)-6-methyltetrahydro-2H-pyran-4-ylcarbamate (>99% ee) and tert-butyl (2R,4S,6R)-2-(3-aminopyridin-4-yl)-6-methyltetrahydro-2H-pyran-4-ylcarbamate (>99% ee).

### Synthesis of (+/−)3-hydroxy-2-methyl-6-(3-nitropyridin-4-yl)dihydro-2H-pyran-4(3H)-one

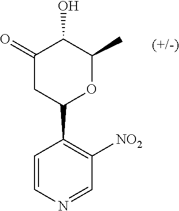

(+/−)4-(6-methyl-4-(triethylsilyloxy)-3,6-dihydro-2H-pyran-2-yl)-3-nitropyridine (1.0 equiv.) was dissolved in DCM (0.2 M) in a flame dried rbf. 3,3-dimethyldioxirane (0.1 M in acetone) (0.5 equiv.) (prepared as in Chem. Ber. 124 (1991) 2377) was added, the reaction capped and stirred on an ice bath, allowing to warm to room temperature for 1.5 hours. 3,3-dimethyldioxirane (0.1 M in acetone) (0.5 equiv.) was added at ˜15° C. and the reaction stirred for 1 hour. 3,3-dimethyldioxirane (0.1 M in acetone) (0.2 equiv.) was added and the reaction stirred at room temperature for 10 min. Cyclohexene (5.0 equiv.) was added and the solution stirred for 20 min. The solvents were removed in vacuo and the residue redissolved in THF (0.1 M). HCl (1.0 M) (2.0 equiv.) was added and the solution stirred for 15 min. NaOH (1.0 M) was added until the pH was ˜9. The mixture was extracted with EtOAc and dried over sodium sulfate, filtered and concentrated. The residue was purified by ISCO using a RediSep column, eluting with 0-100% EtOAc in Heptanes to give (+/−)3-hydroxy-2-methyl-6-(3-nitropyridin-4-yl)dihydro-2H-pyran-4(3H)-one in 43% yield. LC/MS (m/z): 253.0 (MH⁺), Rₜ=0.48 min. ¹H NMR (400 MHz, CHLOROFORM-d) δ ppm 1.55 (d, 3H) 2.61 (t, 1H) 3.15 (dd, 1H) 3.58-3.68 (m, 2H) 3.96 (d, 1H) 5.36 (dd, 1H) 7.89 (d, 1H) 8.91 (d, 1H) 9.24 (s, 1H).

### Synthesis (+/−)4-(benzylamino)-2-methyl-6-(3-nitropyridin-4-yl)tetrahydro-2H-pyran-3-ol

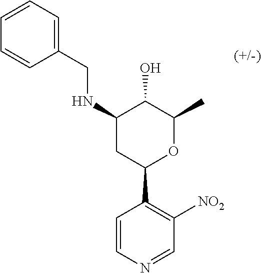 [SMILES: C[C@H]1O[C@@H](c2ccncc2[N+](=O)[O-])C[C@@H](NCc2ccccc2)[C@@H]1O]

(+/−)3-hydroxy-2-methyl-6-(3-nitropyridin-4-yl)dihydro-2H-pyran-4(3H)-one U-was dissolved in MeOH (0.2 M) under N₂ and phenylmethanamine (2.0 equiv.) was added. The reaction was stirred for 2 hours then cooled to −78° C. under N₂ and lithium tetrahydroborate (2.0 M) (1.1 equiv.) was added drop wise. The cooling bath was removed and the reaction stirred for 2 hours allowing to warm to room temperature. The solution was diluted with EtOAc and washed with sat. sodium bicarbonate, brine, dried over sodium sulfate, filtered and concentrated in vacuo to give (+/−)4-(benzylamino)-2-methyl-6-(3-nitropyridin-4-yl)tetrahydro-2H-pyran-3-ol in 43% yield. LC/MS (m/z): 344.2 (MH⁺), Rₜ=0.52 min. ¹H NMR (400 MHz, CHLOROFORM-d) δ ppm 1.35 (d, 3H) 1.52-1.61 (m, 1H) 1.68 (br. s., 1H) 2.49 (d, 1H) 3.19 (d, 1H) 3.33 (m, 2H) 3.51-3.60 (m, 1H) 3.74 (d, J=12.13 Hz, 1H) 4.13 (d, 1H) 5.33 (d, 1H) 7.31 (d, 1H) 7.38 (t, 2H) 7.42-7.47 (m, 2H) 7.85 (d, 1H) 8.82 (d, 1H) 9.23 (s, 1H).

### Synthesis of (+/−)N-benzyl-3-(tert-butyldimethylsilyloxy)-2-methyl-6-(3-nitropyridin-4-yl)tetrahydro-2H-pyran-4-amine

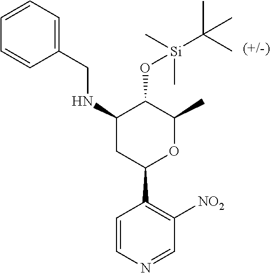 [SMILES: C[C@H]1O[C@@H](c2ccncc2[N+](=O)[O-])C[C@@H](NCc2ccccc2)[C@@H]1O[Si](C)(C)C(C)(C)C]

(+/−)4-(benzylamino)-2-methyl-6-(3-nitropyridin-4-yl)tetrahydro-2H-pyran-3-ol (1.0 equiv) was dissolved in DMF (0.8 M). 1H-imidazole (10.0 equiv.) and tert-butylchlorodimethylsilane (5.0 equiv.) were added and the reaction stirred for 18 hours. The solution was poured into water and extracted with EtOAc, dried over sodium sulfate, filtered and concentrated. The residue was purified by ISCO using a RediSep column eluting with 0-50% EtOAc in Heptanes to give (+/−)N-benzyl-3-(tert-butyldimethylsilyloxy)-2-methyl-6-(3-nitropyridin-4-yl)tetrahydro-2H-pyran-4-amine in 38% yield. LC/MS (m/z): 458.2 (MH⁺), Rₜ=0.94 min. ¹H NMR (400 MHz, CHLOROFORM-d) δ ppm 0.01 (s, 3H) 0.10 (s, 3H) 0.90 (s, 9H) 1.21 (d, 3H) 1.44-1.53 (m, 1H) 2.42-2.50 (m, 1H) 3.12 (d, 1H) 3.49 (dd, 1H) 3.63 (d, 1H) 3.98-4.10 (m, 2H) 5.75 (d, 1H) 7.24-7.43 (m, 5H) 7.83 (d, 1H) 8.78 (d, 1H) 9.17 (s, 1H).

### Synthesis of (+/−) tert-butyl 6-(3-aminopyridin-4-yl)-3-(tert-butyldimethylsilyloxy)-2-methyltetrahydro-2H-pyran-4-ylcarbamate

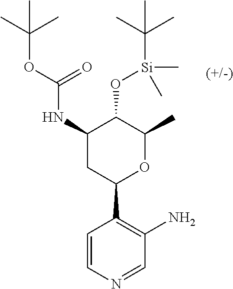 [SMILES: C[C@H]1O[C@@H](c2ccncc2N)C[C@@H](NC(=O)OC(C)(C)C)[C@@H]1O[Si](C)(C)C(C)(C)C]

(+/−)N-benzyl-3-(tert-butyldimethylsilyloxy)-2-methyl-6-(3-nitropyridin-4-yl)tetrahydro-2H-pyran-4-amine (1.0 equiv.) was dissolved in MeOH (0.2 M) and degassed with vacuum to Argon. Palladium hydroxide (0.2 equiv.) was added and the mixture stirred under an H₂ balloon for 2 hours. The H₂ was removed by vacuum, the mixture placed under N₂, di-tert-butyl dicarbonate (2.0 equiv.) was added and the mixture stirred for 16 hours. The mixture was filtered through a 1 uM PTFE ACRODISC CR filter and concentrated. The crude residue was purified by ISCO using a RediSep column eluting with 0-100% EtOAc in Heptanes to give (+/−) tert-butyl 6-(3-aminopyridin-4-yl)-3-(tert-butyldimethylsilyloxy)-2-methyltetrahydro-2H-pyran-4-ylcarbamate in 85% yield. LC/MS (m/z): 338.2 (M-Boc+H⁺), Rₜ=0.62 min. ¹H NMR (400 MHz, CHLOROFORM-d) δ ppm 0.12 (d, J=4.30 Hz, 6H) 0.92 (s, 9H) 1.28 (d, 3H) 1.46 (s, 9H) 1.94-2.03 (m, 1H) 2.56 (d, 1H) 3.52 (dd, 1H) 3.63-3.72 (m, 1H) 3.90-3.95 (m, 1H) 4.16 (br. s., 2H) 4.70 (d, 1H) 4.99 (br. s., 1H) 7.00 (d, 1H) 7.98 (d, 1H) 8.04 (s, 1H). The material was separated via chiral HPLC (IC column, heptane:EtOH 95:05) to give tert-butyl (2S,3R,4S,6S)-6-(3-aminopyridin-4-yl)-3-(tert-butyldimethylsilyloxy)-2-methyltetrahydro-2H-pyran-4-ylcarbamate (>99% ee) and tert-butyl (2R,3S,4R,6R)-6-(3-aminopyridin-4-yl)-3-(tert-butyldimethylsilyloxy)-2-methyltetrahydro-2H-pyran-4-ylcarbamate (>99% ee).

### Synthesis of Cis (+/−)4-(5,6-dimethyl-4-(trimethylsilyloxy)-3,6-dihydro-2H-pyran-2-yl)-3-nitropyridine

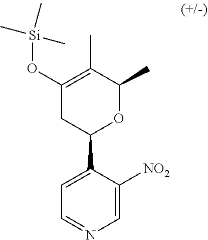 [SMILES: CC1=C(O[Si](C)(C)C)C[C@H](c2ccncc2[N+](=O)[O-])O[C@@H]1C]

(E)-trimethyl(3-methylpenta-1,3-dien-2-yloxy)silane (2.7 equiv.), 3-nitroisonicotinaldehyde (1.0 equiv.), and tris(6,6,7,7,8,8,8-heptafluoro-2,2-dimethyl-3,5-octanedionato) europium (0.05 equiv.) were dissolved in CHCl₃ (1.13 M) in a flame dried rbf and stirred at 60° C. under Argon for 1.5 hours. The heat was turned off and the reaction stirred overnight at room temperature. The volatiles were removed in vacuo and the red liquid was purified by ISCO using a RediSep column eluting with 0-50% EtOAc in Heptanes to give Cis (+/−)4-(5,6-dimethyl-4-(trimethylsilyloxy)-3,6-dihydro-2H-pyran-2-yl)-3-nitropyridine in 68% yield. LC/MS (m/z): 251.0 (M-SiMe₃+H⁺), Rₜ=0.73 min. ¹H NMR (400 MHz, CHLOROFORM-d) δ ppm 0.20 (s, 9H) 1.32 (d, 3H) 1.58 (s, 3H) 2.15-2.27 (m, 1H) 2.46 (d, 1H) 4.27-4.35 (m, 1H) 5.21 (dd, 1H) 7.83 (d, 1H) 8.84 (d, 1H) 9.17 (s, 1H).

### Synthesis of (+/−)-2,3-dimethyl-6-(3-nitropyridin-4-yl)dihydro-2H-pyran-4(3H)-one

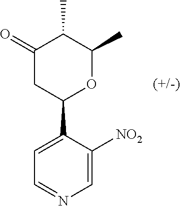 [SMILES: C[C@H]1O[C@@H](c2ccncc2[N+](=O)[O-])CC(=O)[C@@H]1C]

To a solution of Cis (+/−)4-(5,6-dimethyl-4-(trimethylsilyloxy)-3,6-dihydro-2H-pyran-2-yl)-3-nitropyridine (1.0 equiv) in THF (0.28M) was added 1N HCl (1.0 equiv.). After stirring for 1 hour 1N NaOH (1.0 equiv) was added and the volatiles were removed in vacuo. The residue was partitioned between EtOAc and NaHCO_{3(sat.)}, washed with NaCl_{(sat.)}, dried over Na₂SO₄, filtered, concentrated and purified by RP-HPLC (to remove minor diastereomer) to yield (+/−)-2,3-dimethyl-6-(3-nitropyridin-4-yl)dihydro-2H-pyran-4(3H)-one in 73% yield. LC/MS (m/z): 251.2 (MH⁺) Rₜ=0.72 min.

### Synthesis of (+/−)-2,3-dimethyl-6-(3-nitropyridin-4-yl)tetrahydro-2H-pyran-4-ol

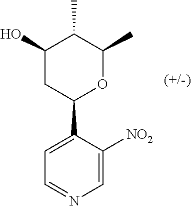 [SMILES: C[C@H]1[C@H](O)C[C@H](c2ccncc2[N+](=O)[O-])O[C@@H]1C]

To a solution of (+/−)-2,3-dimethyl-6-(3-nitropyridin-4-yl)dihydro-2H-pyran-4(3H)-one (1.0 equiv.) in MeOH (0.05 M) at 0° C. was added sodium borohydride (1.0 equiv.). After stirring in the ice bath for 60 minutes, water was added to quench and the volatiles were removed in vacuo. The residue was portioned between EtOAc and NaCl(sat.), separated, dried over MgSO₄, filtered, concentrated and purified by ISCO SiO₂ chromatography (20-60% EtOAc/n-heptanes gradient) to yield 2,4,6 cis-(+/−)-2,3-dimethyl-6-(3-nitropyridin-4-yl)tetrahydro-2H-pyran-4-ol in 75% yield. LC/MS (m/z): 253.0 (MH⁺), Rₜ=0.64 min. A diasteromeric-(+/−)-2,3-dimethyl-6-(3-nitropyridin-4-yl)tetrahydro-2H-pyran-4-ol was also isolated in 20% yield. LC/MS (m/z): 253.0 (MH⁺), Rₜ=0.65 min.

### Synthesis of 4-((2R,4R,5R,6R)-4-(tert-butyldimethylsilyloxy)-5,6-dimethyltetrahydro-2H-pyran-2-yl)-3-nitropyridine+enantiomer

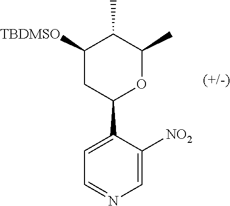 [SMILES: C[C@@H]1[C@@H](C)O[C@@H](c2ccncc2[N+](=O)[O-])C[C@H]1O[Si](C)(C)C(C)(C)C]

To a solution of (+/−)-2,3-dimethyl-6-(3-nitropyridin-4-yl)tetrahydro-2H-pyran-4-ol (1.0 equiv) in DMF (0.8 M) was added 1H-imidazole (5.0 equiv.) and tert-butylchlorodimethylsilane (2.0 equiv.) and the reaction was stirred for 18 hours. The solution was poured into water and extracted with EtOAc, dried over sodium sulfate, filtered and concentrated. The residue was purified by ISCO SiO₂ chromatography (0-100% EtOAc in Heptanes gradient) to yield 4-((2R,4R,5R,6R)-4-(tert-butyldimethylsilyloxy)-5,6-dimethyltetrahydro-2H-pyran-2-yl)-3-nitropyridine+enantiomer. LC/MS (m/z): 367.2 (MH⁺), Rₜ=1.38 min.

### Synthesis of 4-((2R,4R,5R,6R)-4-(tert-butyldimethylsilyloxy)-5,6-dimethyltetrahydro-2H-pyran-2-yl)pyridin-3-amine+enantiomer

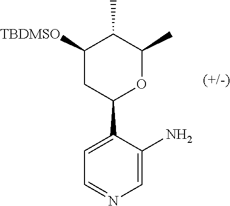 [SMILES: C[C@@H]1[C@@H](C)O[C@@H](c2ccncc2N)C[C@H]1O[Si](C)(C)C(C)(C)C]

4-((2R,4R,5R,6R)-4-(tert-butyldimethylsilyloxy)-5,6-dimethyltetrahydro-2H-pyran-2-yl)-3-nitropyridine+enantiomer (1.0 equiv.) was dissolved in EtOH (0.05 M) and degassed with vacuum to Argon. Palladium on carbon (0.1 equiv.) was added and the mixture placed under an H₂ balloon for 16 hours. The mixture was filtered through a pad of celite, concentrated and purified by ISCO SiO2 chromaography (0-10% MeOH/CH₂Cl₂ gradient) to give 4-((2R,4R,5R,6R)-4-(tert-butyldimethylsilyloxy)-5,6-dimethyltetrahydro-2H-pyran-2-yl)pyridin-3-amine+enantiomer_ in 75% yield. LC/MS (m/z): 337.1 (MH⁺), Rₜ=0.98 min. The material could be resolved with chiral chromatography (analytical conditions, 90/10 n-heptanes/isopropylalcohol, 1 mL/min, IC column, Rₜ's=7.24 and 8.98 min).

### Synthesis of (+/−)3-hydroxy-2,3-dimethyl-6-(3-nitropyridin-4-yl)dihydro-2H-pyran-4(3H)-one

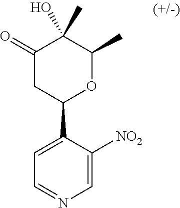 [SMILES: C[C@H]1O[C@@H](c2ccncc2[N+](=O)[O-])CC(=O)[C@]1(C)O]

(+/−)4-(5,6-dimethyl-4-(trimethylsilyloxy)-3,6-dihydro-2H-pyran-2-yl)-3-nitropyridine (1.0 equiv.) was dissolved in DCM (0.2 M) in a flame dried rbf. 3,3-dimethyldioxirane (0.1 M in acetone) (0.5 equiv.) (prepared as in Chem. Ber. 124 (1991) 2377) was added, the reaction capped and stirred on an ice bath, allowing to warm to room temperature for 1.5 hours. 3,3-dimethyldioxirane (0.1 M in acetone) (0.5 equiv.) was added at ˜15° C. and the reaction stirred for 1 hour. Cyclohexene (5.0 equiv.) was added and the solution stirred for 20 min. The solvents were removed in vacuo and the residue redissolved in THF (0.1 M). HCl (1.0 M) (2.0 equiv.) was added and the solution stirred for 15 min. NaOH (1.0 M) was added until the pH was ˜9. The mixture was extracted with EtOAc and dried over sodium sulfate, filtered and concentrated. The residue was purified by ISCO using a RediSep column, eluting with 0-100% EtOAc in Heptanes to give (+/−) (3-hydroxy-2,3-dimethyl-6-(3-nitropyridin-4-yl)dihydro-2H-pyran-4(3H)-one in 62% yield. LC/MS (m/z): 267.0 (MH⁺), Rₜ=0.55 min. ¹H NMR (400 MHz, DMSO-d₆) δ ppm 1.20 (d, 3H) 1.26 (s, 3H) 2.77 (dd, 1H) 2.92 (dd, 1H) 3.69 (q, 1H) 5.27 (dd, 1H) 7.88 (d, 1H) 8.93 (d, 1H) 9.16 (s, 1H).

### Synthesis of (+/−)2,3-dimethyl-6-(3-nitropyridin-4-yl)tetrahydro-2H-pyran-3,4-diol

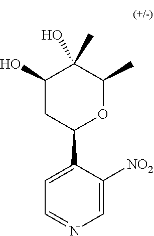 [SMILES: C[C@H]1O[C@@H](c2ccncc2[N+](=O)[O-])C[C@@H](O)[C@]1(C)O]

3-hydroxy-2,3-dimethyl-6-(3-nitropyridin-4-yl)dihydro-2H-pyran-4(3H)-one (1 equiv.) was dissolved in Ethanol (0.2 M) and cooled to 0° C. on an ice bath. Sodium tetrahydroborate (1.2 equiv.) was added and the reaction stirred for 2 hours allowing to warm to room temperature. The mixture was diluted with EtOAc, washed with water, dried over sodium sulfate, filtered and concentrated. The residue was purified by ISCO using a RediSep column eluting with 0-100% EtOAc in Heptanes to yield 2,3-dimethyl-6-(3-nitropyridin-4-yl)tetrahydro-2H-pyran-3,4-diol in 67% yield. LC/MS (m/z): 269.1 (MH⁺), Rₜ=0.46 min. ¹H NMR (400 MHz, CHLOROFORM-d) δ ppm 1.25 (s, 3H) 1.27 (d, 3H) 1.51 (q, 1H) 2.38 (ddd, 1H) 3.51 (q, 1H) 3.90 (dd, 1H) 5.18 (dd, 1H) 7.77 (d, 1H) 8.82 (d, 1H) 9.17 (s, 1H).

### Synthesis of (+/−)4-(tert-butyldimethylsilyloxy)-2,3-dimethyl-6-(3-nitropyridin-4-yl)tetrahydro-2H-pyran-3-ol

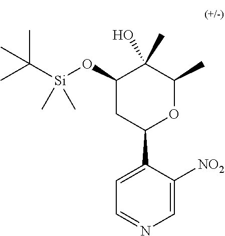 [SMILES: C[C@H]1O[C@@H](c2ccncc2[N+](=O)[O-])C[C@@H](O[Si](C)(C)C(C)(C)C)[C@]1(C)O]

2,3-dimethyl-6-(3-nitropyridin-4-yl)tetrahydro-2H-pyran-3,4-diol (1.0 equiv) was dissolved in DMF (0.8 M). 1H-imidazole (5.0 equiv.) and tert-butylchlorodimethylsilane (2.0 equiv.) were added and the reaction stirred for 18 hours. The solution was poured into water and extracted with EtOAc, dried over sodium sulfate, filtered and concentrated. The residue was purified by ISCO using a RediSep column eluting with 0-100% EtOAc in Heptanes to yield 4-(tert-butyldimethylsilyloxy)-2,3-dimethyl-6-(3-nitropyridin-4-yl)tetrahydro-2H-pyran-3-ol in 86% yield. LC/MS (m/z): 383.1 (MH⁺) Rₜ=1.17 min. ¹H NMR (400 MHz, CHLOROFORM-d) δ ppm 0.11 (s, 3H) 0.15 (s, 3H) 0.90 (s, 9H) 1.23 (s, 3H) 1.27 (d, 3H), 1.42-1.54 (m, 1H), 1.96 (br s, 1H), 2.26 (m, 1H) 3.53 (q, 1H) 3.84 (dd, 1H) 5.14 (dd, 1H) 7.79 (d, 1H) 8.82 (d, 1H) 9.18 (s, 1H).

### Synthesis of (+/−)6-(3-aminopyridin-4-yl)-4-(tert-butyldimethylsilyloxy)-2,3-dimethyltetrahydro-2H-pyran-3-ol

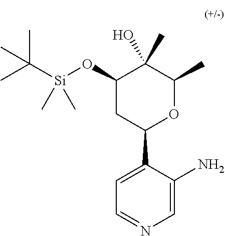 [SMILES: C[C@H]1O[C@@H](c2ccncc2N)C[C@@H](O[Si](C)(C)C(C)(C)C)[C@]1(C)O]

4-(tert-butyldimethylsilyloxy)-2,3-dimethyl-6-(3-nitropyridin-4-yl)tetrahydro-2H-pyran-3-ol (1.0 equiv.) was dissolved in MeOH (0.2 M) and degassed with vacuum to Argon. Palladium hydroxide (0.2 equiv.) was added and the mixture placed under an H₂ balloon for 2 hours. The mixture was filtered through a 1 uM PTFE ACRODISC CR filter and concentrated to give 6-(3-aminopyridin-4-yl)-4-(tert-butyldimethylsilyloxy)-2,3-dimethyltetrahydro-2H-pyran-3-ol in 84% yield. LC/MS (m/z): 353.2 (MH⁺) Rₜ=0.81 min. ¹H NMR (400 MHz, CHLOROFORM-d) δ ppm 0.11 (d, 6H) 0.91 (s, 9H) 1.22 (s, 3H) 1.27 (d, 3H) 1.89 (ddd, 1H) 1.98-2.09 (m, 1H) 2.14 (br. s., 1H) 3.51 (q, 1H) 3.78 (dd, 1H) 4.27 (br. s., 2H) 4.53 (dd, 1H) 6.93 (d, 1H) 7.98 (d, 1H) 8.04 (s, 1H). The material was separated via chiral HPLC (OJ-H column, heptane:EtOH 95:05) to give (2R,3R,4R,6R)-6-(3-aminopyridin-4-yl)-4-(tert-butyldimethylsilyloxy)-2,3-dimethyltetrahydro-2H-pyran-3-ol (>99% ee) and (2S,3S,4S,6S)-6-(3-aminopyridin-4-yl)-4-(tert-butyldimethylsilyloxy)-2,3-dimethyltetrahydro-2H-pyran-3-ol (>99% ee).

### Synthesis of tert-butyldimethyl(4-methylpenta-1,3-dien-2-yloxy)silane

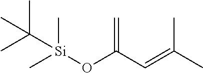

To a 2 neck round bottom flask equipped with an internal thermometer and a magnetic stir bar was added 4-methylpent-3-en-2-one (1.0 equiv.), THF (2.0 M), and triethylamine (1.5 equiv.). The mixture was cooled to 0° C. under N₂ and tert-butyldimethylsilyl trifluoromethanesulfonate (1.0 equiv.) was added over ˜30 min. via addition funnel. The reaction was stirred allowing to warm to room temperature for 2 hours, quenched with sat. sodium bicarbonate, and extracted with heptanes. The combined organics were washed with water, brine, dried over sodium sulfate, filtered and concentrated. The crude liquid was distilled (110° C./10 mm Hg) to give tert-butyldimethyl(4-methylpenta-1,3-dien-2-yloxy)silane in 71% yield. ¹H NMR (400 MHz, CHLOROFORM-d) δ ppm 0.18 (s, 6H) 0.95 (s, 9H) 1.78 (s, 3H) 1.91 (s, 3H) 4.17 (s, 1H) 4.31 (s, 1H) 5.57 (br. s., 1H).

### Synthesis of (+/−)4-(4-(tert-butyldimethylsilyloxy)-6,6-dimethyl-3,6-dihydro-2H-pyran-2-yl)-3-nitropyridine

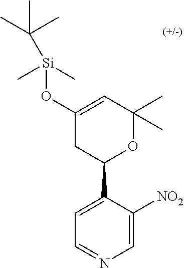 [SMILES: CC1(C)C=C(O[Si](C)(C)C(C)(C)C)C[C@H](c2ccncc2[N+](=O)[O-])O1]

tert-butyldimethyl(4-methylpenta-1,3-dien-2-yloxy)silane (2.0 equiv.), 3-nitroisonicotinaldehyde (1.0 equiv.), and tris(6,6,7,7,8,8,8-heptafluoro-2,2-dimethyl-3,5-octanedionato) europium (0.05 equiv.) were dissolved in CHCl₃ (1.13 M) in a flame dried rbf and stirred at 60° C. under Argon for 1 hour. The heat was turned off and the reaction and stirred overnight at room temperature. The volatiles were removed in vacuo and the liquid was purified by ISCO using a RediSep column eluting with 0-20% EtOAc in Heptanes to give (+/−)4-(4-(tert-butyldimethylsilyloxy)-6,6-dimethyl-3,6-dihydro-2H-pyran-2-yl)-3-nitropyridine in 70% yield. LC/MS (m/z): 365.1 (MH⁺), Rₜ=1.32 min. ¹H NMR (400 MHz, CHLOROFORM-d) δ ppm 0.18 (d, 6H) 0.93 (s, 9H) 1.31-1.39 (m, 6H) 2.13 (ddd, 1H) 2.42 (dd, 1H) 4.90 (d, 1H) 5.42 (dd, 1H) 7.88 (d, 1H) 8.91 (d, 1H) 9.24 (s, 1H).

### Synthesis of Trans (+/−) (3S,6R)-3-hydroxy-2,2-dimethyl-6-(3-nitropyridin-4-yl)dihydro-2H-pyran-4(3H)-one

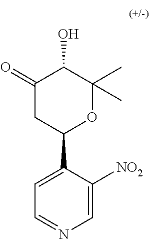 [SMILES: CC1(C)O[C@@H](c2ccncc2[N+](=O)[O-])CC(=O)[C@@H]1O]

To a 3 neck round bottom flask fitted with an internal thermometer was added sodium bicarbonate (5.0 equiv.), water (0.24 M), acetone (10.0 equiv.), and (+/−)4-(4-(tert-butyldimethylsilyloxy)-6,6-dimethyl-3,6-dihydro-2H-pyran-2-yl)-3-nitropyridine dissolved in ethyl acetate (0.24 M). Oxone (1.0 equiv.) dissolved in water (0.24 M) was added drop wise over 1 hour, keeping the internal temperature ˜20° C. The mixture was diluted with EtOAc and washed with brine, the organic layer was concentrated in vacuo. The residue redissolved in THF (0.1 M). HCl (1.0 M) (2.0 equiv.) was added and the solution stirred for 15 min. NaOH (1.0 M) was added until the pH was ˜9. The mixture was extracted with EtOAc and dried over sodium sulfate, filtered and concentrated. The residue was purified by ISCO using a RediSep column, eluting with 0-100% EtOAc in Heptanes to give Trans (+/−) (3-hydroxy-2,2-dimethyl-6-(3-nitropyridin-4-yl)dihydro-2H-pyran-4(3H)-one in 36% yield. LC/MS (m/z): 267.0 (MH⁺), Rₜ=0.57 min. ¹H NMR (400 MHz, CHLOROFORM-d) δ ppm 1.18 (s, 3H) 1.54 (s, 3H) 2.50-2.59 (m, 1H) 3.08 (dd, 1H) 3.71 (d, 1H) 4.14 (d, 1H) 5.52 (dd, 1H) 7.90 (d, 1H) 8.89 (d, 1H) 9.19 (s, 1H).

### Synthesis of (+/−)2,2-dimethyl-6-(3-nitropyridin-4-yl)tetrahydro-2H-pyran-3,4-diol

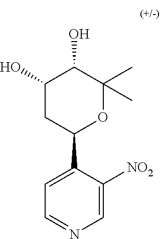 [SMILES: CC1(C)O[C@@H](c2ccncc2[N+](=O)[O-])C[C@H](O)[C@@H]1O]

(+/−)3-hydroxy-2,2-dimethyl-6-(3-nitropyridin-4-yl)dihydro-2H-pyran-4(3H)-one (1.0 equiv.) was dissolved in Ethanol (0.2 M) and cooled to 0° C. on an ice bath. Sodium borohydride (1.2 equiv.) was added and the reaction stirred for 2 hours allowing to warm to room temperature. The mixture was diluted with EtOAc, washed with water, dried over sodium sulfate, filtered and concentrated. The crude orange residue was purified by ISCO using a RediSep column eluting with 0-100% EtOAc in Heptanes to yield (+/−)2,2-dimethyl-6-(3-nitropyridin-4-yl)tetrahydro-2H-pyran-3,4-diol in 93% yield. LC/MS (m/z): 269.0 (MH⁺), Rₜ=0.46 min. ¹H NMR (400 MHz, DMSO-d₆) δ ppm 1.16 (s, 3H) 1.32 (s, 3H) 1.70 (ddd, 1H) 1.99-2.06 (m, 1H) 3.20 (br. s., 1H) 3.96 (d, 1H) 4.78 (d, 2H) 5.34 (dd, 1H) 7.75 (d, 1H) 8.83 (d, 1H) 9.05 (s, 1H).

### Synthesis of (+/−)4-(tert-butyldimethylsilyloxy)-2,2-dimethyl-6-(3-nitropyridin-4-yl)tetrahydro-2H-pyran-3-ol

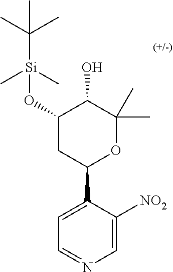

(+/−)2,2-dimethyl-6-(3-nitropyridin-4-yl)tetrahydro-2H-pyran-3,4-diol (1.0 equiv) was dissolved in DMF (0.8 M). 1H-imidazole (5 equiv.) and tert-butylchlorodimethylsilane (2.0 equiv.) were added and the reaction stirred at ambient temperature for 18 hours. The solution was poured into water and extracted with EtOAc, dried over sodium sulfate, filtered and concentrated. The residue was purified by ISCO using a RediSep column eluting with 0-50% EtOAc in Heptanes to give (+/−)4-(tert-butyldimethylsilyloxy)-2,2-dimethyl-6-(3-nitropyridin-4-yl)tetrahydro-2H-pyran-3-ol in 77% yield. LC/MS (m/z): 383.2 (MH⁺), Rₜ=1.17 min. ¹H NMR (400 MHz, DMSO-d₆) δ ppm 0.09 (d, 6H) 0.92 (s, 9H) 1.19 (s, 3H) 1.31 (s, 3H) 1.65-1.74 (m, 1H) 1.94 (ddd, 1H) 3.25 (dd, 1H) 4.06 (d, 1H) 4.88 (d, 1H) 5.38 (d, 1H) 7.78 (d, 1H) 8.84 (d, 1H) 9.07 (s, 1H).

### Synthesis of (+/−)6-(3-aminopyridin-4-yl)-4-(tert-butyldimethylsilyloxy)-2,2-dimethyltetrahydro-2H-pyran-3-ol

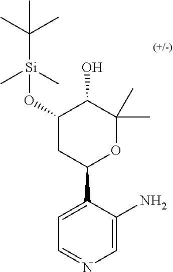

(+/−)4-(tert-butyldimethylsilyloxy)-2,2-dimethyl-6-(3-nitropyridin-4-yl)tetrahydro-2H-pyran-3-ol (1.0 equiv.) was dissolved in MeOH (0.2 M) and degassed with Argon. Palladium hydroxide (0.2 equiv.) was added and the mixture placed under an H₂ balloon for 2 hours. The mixture was filtered through a 1 uM PTFE ACRODISC CR filter and concentrated to give (+/−)6-(3-aminopyridin-4-yl)-4-(tert-butyldimethylsilyloxy)-2,2-dimethyltetrahydro-2H-pyran-3-ol in 74% yield. LC/MS (m/z): 353.1 (MH⁺), Rₜ=0.86 min. ¹H NMR (400 MHz, DMSO-d₆) δ ppm 0.07 (s, 3H) 0.10 (s, 3H) 0.90 (s, 9H) 1.18 (s, 3H) 1.37 (s, 3H) 1.70-1.76 (m, 1H) 1.91-2.00 (m, 1H) 3.25 (dd, 1H) 3.32 (s, 1H) 4.08 (d, 1H) 4.78 (d, 1H) 4.84 (d, 1H) 4.95 (s, 1H) 6.94 (d, 1H) 7.77 (d, 1H) 7.97 (s, 1H). The material was separated via chiral HPLC (OD-H column, heptane:IPA 90:10) to give (3R,4R,6S)-6-(3-aminopyridin-4-yl)-4-(tert-butyldimethylsilyloxy)-2,2-dimethyltetrahydro-2H-pyran-3-ol (>99% ee) and (3S,4S,6R)-6-(3-aminopyridin-4-yl)-4-(tert-butyldimethylsilyloxy)-2,2-dimethyltetrahydro-2H-pyran-3-ol (>99% ee).

### Synthesis of (+/−)4-(5,6-dimethyl-4,5-bis(trimethylsilyloxy)-5,6-dihydro-2H-pyran-2-yl)-3-nitropyridine

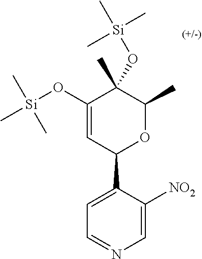 [SMILES: C[C@H]1O[C@@H](c2ccncc2[N+](=O)[O-])C=C(O[Si](C)(C)C)[C@]1(C)O[Si](C)(C)C]

(+/−)3-hydroxy-2,3-dimethyl-6-(3-nitropyridin-4-yl)dihydro-2H-pyran-4(3H)-one (1.0 equiv.) was dissolved in dry THF (0.054 M) in a flame dried 3-neck rbf under N₂. The solution was cooled to −78° C. and chlorotrimethylsilane (10.0 equiv.) was added. KHMDS (0.5 M in toluene) (3.0 equiv.) was added keeping the internal temperature <−45° C. The reaction was stirred at −70° C. for 2 hours. The reaction was complete by TLC (4:1 Heptanes:EtOAc). Sat. sodium bicarbonate was added, the cooling bath removed, and the mixture was stirred as it was warmed to room temperature over 1 hour. Heptanes was added and the mixture washed with water, brine, the organics were dried over sodium sulfate, filtered and concentrated to give (+/−)4-(5,6-dimethyl-4,5-bis(trimethylsilyloxy)-5,6-dihydro-2H-pyran-2-yl)-3-nitropyridine in 91% yield. ¹H NMR (400 MHz, CHLOROFORM-d) δ ppm 0.16 (s, 9H) 0.18 (s, 9H) 1.26 (d, 3H) 1.35 (s, 3H) 3.82 (q, 1H) 4.75 (d, 1H) 5.81 (d, 1H) 7.73 (d, 1H) 8.81 (d, 1H) 9.16 (s, 1H).

### Synthesis of (+/−)-5-(tert-butyldimethylsilyloxy)-3-hydroxy-2,3-dimethyl-6-(3-nitropyridin-4-yl)dihydro-2H-pyran-4(3H)-one

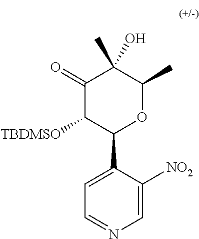 [SMILES: C[C@H]1O[C@@H](c2ccncc2[N+](=O)[O-])[C@H](O[Si](C)(C)C(C)(C)C)C(=O)[C@]1(C)O]

A solution of (+/−)4-(5,6-dimethyl-4,5-bis(trimethylsilyloxy)-5,6-dihydro-2H-pyran-2-yl)-3-nitropyridine in CH₂Cl₂ (0.2 M) at 0° C. was treated with DMDO until all of the SM was consumed as judged by LC/MS analysis. At this time cyclohexene was added to consume any remaining oxidant and the volatiles were removed in vacuo. The residue was dissolved in 3:1 THF/1N HCl. After stirring at rt for one hour, the reaction was diluted with EtOAc, was washed with NaHCO_{3(sat.)}, with NaCl_{(sat.)}, dried over MgSO₄, filtered and concentrated to yield crude hydroxyl ketone along with pyridine N-oxide byproduct. The residue was dissolved in DMF and treated with imidazole (5 equiv.) and TBDMSCl (2.2 equiv). Upon standing for 18 hours, the solution was diluted with EtOAc, washed with H₂O (3×), with NaCl_{(sat.)}, dried over MgSO₄, filtered and concentrated and purified by ISCO SiO₂ chromatography (20% EtOAc/n-heptanes) to yield (+/−)-5-(tert-butyldimethylsilyloxy)-3-hydroxy-2,3-dimethyl-6-(3-nitropyridin-4-yl)dihydro-2H-pyran-4(3H)-one (20%). LC/MS (m/z): 397.1 (MH⁺), Rₜ=1.08 min.

### Synthesis of (+/−)-5-(tert-butyldimethylsilyloxy)-2,3-dimethyl-6-(3-nitropyridin-4-yl)tetrahydro-2H-pyran-3,4-diol

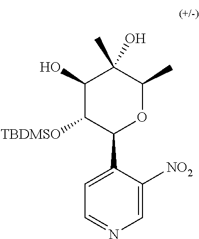 [SMILES: C[C@H]1O[C@@H](c2ccncc2[N+](=O)[O-])[C@H](O[Si](C)(C)C(C)(C)C)[C@@H](O)[C@]1(C)O]

To a solution of (+/−)-5-(tert-butyldimethylsilyloxy)-3-hydroxy-2,3-dimethyl-6-(3-nitropyridin-4-yl)dihydro-2H-pyran-4(3H)-one (1.0 equiv.) in MeOH (0.05 M) at 0° C. was added sodium borohydride (1.0 equiv.). After stirring in the ice bath for 10 minutes, water was added to quench and the volatiles were removed in vacuo. The residue was portioned between EtOAc and NaCl_{(sat.)}, separated, dried over MgSO₄, filtered and the volatiles were removed in vacuo to yield (+/−)-5-(tert-butyldimethylsilyloxy)-2,3-dimethyl-6-(3-nitropyridin-4-yl)tetrahydro-2H-pyran-3,4-diol in 95% yield. LC/MS (m/z): 399.2 (MH⁺) Rₜ=0.99 min.

### Synthesis of (+/−)-6-(3-aminopyridin-4-yl)-5-(tert-butyldimethylsilyloxy)-2,3-dimethyltetrahydro-2H-pyran-3,4-diol

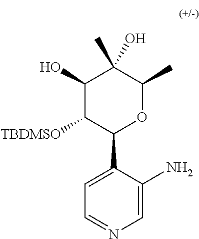 [SMILES: C[C@H]1O[C@@H](c2ccncc2N)[C@H](O[Si](C)(C)C(C)(C)C)[C@@H](O)[C@]1(C)O]

(+/−)-5-(tert-butyldimethylsilyloxy)-2,3-dimethyl-6-(3-nitropyridin-4-yl)tetrahydro-2H-pyran-3,4-diol (1.0 equiv.) was dissolved in EtOH (0.05 M) and degassed with vacuum to Argon. Palladium on carbon (0.1 equiv.) was added and the mixture placed under an H₂ balloon for 16 hours. The mixture was filtered through a pad of celite and concentrated to give (+/−)-6-(3-aminopyridin-4-yl)-5-(tert-butyldimethylsilyloxy)-2,3-dimethyltetrahydro-2H-pyran-3,4-diol in 99% yield. LC/MS (m/z): 369.3 (MH⁺) Rₜ=0.60 min.

### Synthesis of (+/−)5-fluoro-2,3-dimethyl-6-(3-nitropyridin-4-yl)-3-(trimethylsilyloxy)dihydro-2H-pyran-4(3H)-one

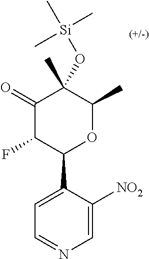 [SMILES: C[C@H]1O[C@@H](c2ccncc2[N+](=O)[O-])[C@H](F)C(=O)[C@]1(C)O[Si](C)(C)C]

(+/−)4-(5,6-dimethyl-4,5-bis(trimethylsilyloxy)-5,6-dihydro-2H-pyran-2-yl)-3-nitropyridine (1.0 equiv.) was dissolved in dry ACN (0.24 M) under N₂ and 1-(chloromethyl)-4-fluoro-1,4-diazoniabicyclo[2.2.2]octane tetrafluoroborate (2.0 equiv.) was added in a single portion. The reaction was stirred at room temperature for 2 hours and then diluted with EtOAc, washed with water, brine, dried over sodium sulfate, filtered and concentrated. The residue was purified by ISCO using a RediSep column eluting with 0-100% EtOAc in Heptanes to give (+/−)5-fluoro-2,3-dimethyl-6-(3-nitropyridin-4-yl)-3-(trimethylsilyloxy)dihydro-2H-pyran-4(3H)-one in 82% yield. LC/MS (m/z): 357.1 (MH⁺), Rₜ=1.10 min. ¹H NMR (400 MHz, CHLOROFORM-d) δ ppm 0.20 (s, 9H) 1.31 (d, 3H) 1.42 (s, 3H) 3.69-3.75 (m, 1H) 5.01-5.17 (m, 1H) 5.28 (dd, 1H) 7.64 (d, 1H) 8.88 (d, 1H) 9.10 (s, 1H).

### Synthesis of (+/−)-6-(3-aminopyridin-4-yl)-5-fluoro-2,3-dimethyl-3-(trimethylsilyloxy)dihydro-2H-pyran-4(3H)-one

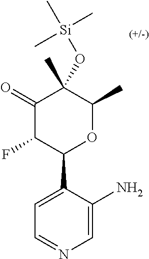 [SMILES: C[C@H]1O[C@@H](c2ccncc2N)[C@H](F)C(=O)[C@]1(C)O[Si](C)(C)C]

To a solution of (+/−)5-fluoro-2,3-dimethyl-6-(3-nitropyridin-4-yl)-3-(trimethylsilyloxy)dihydro-2H-pyran-4(3H)-one in acetic acid (0.15 M) was added iron dust (6.0 equiv). The solution was stirred vigorously for one hour, at which time it was diluted with EtOAc, filtered through a pad of celite and the volatiles were removed in vacuo. The residue was portioned between EtOAc and Na₂CO_{3(sat.)}, separated, washed further with Na₂CO_{3(sat.)}, with NaCl_{(sat.)}, dried over MgSO₄, filtered and concentrated to yield (+/−)-6-(3-aminopyridin-4-yl)-5-fluoro-2,3-dimethyl-3-(trimethylsilyloxy)dihydro-2H-pyran-4(3H)-one (90%). LC/MS (m/z): 327.2 (MH⁺), Rₜ=0.78 min.

### Synthesis of (+/−)5-fluoro-3-hydroxy-2,3-dimethyl-6-(3-nitropyridin-4-yl)dihydro-2H-pyran-4(3H)-one

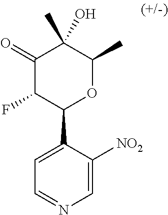 [SMILES: C[C@H]1O[C@@H](c2ccncc2[N+](=O)[O-])[C@H](F)C(=O)[C@]1(C)O]

(+/−)5-fluoro-2,3-dimethyl-6-(3-nitropyridin-4-yl)-3-(trimethylsilyloxy)dihydro-2H-pyran-4(3H)-one (1.0 equiv.) was dissolved in THF/MeOH (2:1) (0.2 M) and HCl (6 M) (7.5 equiv.) was added. The reaction was stirred at room temperature for 1 hour. The volatiles were removed in vacuo and the liquid was diluted with EtOAc and washed with sat. sodium bicarbonate. The aqueous layer was extracted with EtOAc and the combined organics were dried over sodium sulfate, filtered and concentrated to give (+/−)5-fluoro-3-hydroxy-2,3-dimethyl-6-(3-nitropyridin-4-yl)dihydro-2H-pyran-4(3H)-one in 96% yield. LC/MS (m/z): 285.0 (MH⁺), Rₜ=0.59 min.

### Synthesis of (+/−)5-fluoro-2,3-dimethyl-6-(3-nitropyridin-4-yl)tetrahydro-2H-pyran-3,4-diol

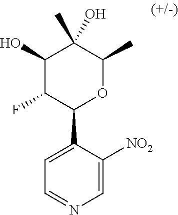 [SMILES: C[C@H]1O[C@@H](c2ccncc2[N+](=O)[O-])[C@H](F)[C@@H](O)[C@]1(C)O]

(+/−)5-fluoro-3-hydroxy-2,3-dimethyl-6-(3-nitropyridin-4-yl)dihydro-2H-pyran-4(3H)-one (1.0 equiv.) was dissolved in MeOH (0.2 M) and cooled to 0° C. on an ice bath. Sodium tetrahydroborate (1.2 equiv.) was added and the reaction stirred for 30 min. Water was added and the mixture was extracted with EtOAc, washed with water, dried over sodium sulfate, filtered and concentrated. The residue was purified by ISCO using a RediSep column eluting with 0-50% (10% MeOH in DCM) in DCM to give (+/−)5-fluoro-2,3-dimethyl-6-(3-nitropyridin-4-yl)tetrahydro-2H-pyran-3,4-diol in 36% yield. LC/MS (m/z): 287.1 (MH⁺), Rₜ=0.51 min.

### Synthesis of (+/−)4-(tert-butyldimethylsilyloxy)-5-fluoro-2,3-dimethyl-6-(3-nitropyridin-4-yl)tetrahydro-2H-pyran-3-ol

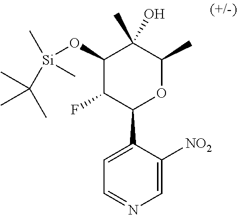 [SMILES: C[C@H]1O[C@@H](c2ccncc2[N+](=O)[O-])[C@H](F)[C@@H](O[Si](C)(C)C(C)(C)C)[C@]1(C)O]

(+/−)5-fluoro-2,3-dimethyl-6-(3-nitropyridin-4-yl)tetrahydro-2H-pyran-3,4-diol (1.0 equiv) was dissolved in DMF (0.8 M). 1H-imidazole (5.0 equiv.) and tert-butylchlorodimethylsilane (2.0 equiv.) were added and the reaction stirred for 16 hours. 1H-imidazole (5.0 equiv.) and tert-butylchlorodimethylsilane (2.0 equiv.) were added and the reaction stirred for 72 hours. The solution was poured into water and extracted with EtOAc, dried over sodium sulfate, filtered and concentrated. The residue was purified by ISCO using a RediSep column eluting with 0-30% EtOAc in Heptanes to give (+/−)4-(tert-butyldimethylsilyloxy)-5-fluoro-2,3-dimethyl-6-(3-nitropyridin-4-yl)tetrahydro-2H-pyran-3-ol in 87% yield. LC/MS (m/z): 401.3 (MH⁺), Rₜ=1.17 min. ¹H NMR (400 MHz, CHLOROFORM-d) δ ppm 9.06 (s, 1H) 8.82 (d, 1H) 7.65 (d, 1H) 5.27 (dd, 1H) 4.15-4.21 (m, 1H) 4.05 (t, 1H) 3.82 (dd, 1H) 3.64 (q, 1H) 1.25-1.29 (m, 3H) 1.21 (s, 3H) 0.92 (s, 9H) 0.15 (s, 3H) 0.10 (s, 3H).

### Synthesis of (+/−)6-(3-aminopyridin-4-yl)-4-(tert-butyldimethylsilyloxy)-5-fluoro-2,3-dimethyltetrahydro-2H-pyran-3-ol

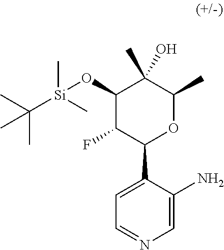 [SMILES: C[C@H]1O[C@@H](c2ccncc2N)[C@H](F)[C@@H](O[Si](C)(C)C(C)(C)C)[C@]1(C)O]

(+/−)4-(tert-butyldimethylsilyloxy)-5-fluoro-2,3-dimethyl-6-(3-nitropyridin-4-yl)tetrahydro-2H-pyran-3-ol (1.0 equiv.) was dissolved in MeOH (0.2 M) and degassed with vacuum to Argon. Palladium hydroxide (0.2 equiv.) was added and the mixture placed under an H₂ balloon for 2 hours. The mixture was filtered through a 1 uM PTFE ACRODISC CR filter and concentrated to give (+/−)6-(3-aminopyridin-4-yl)-4-(tert-butyldimethylsilyloxy)-5-fluoro-2,3-dimethyltetrahydro-2H-pyran-3-ol in 36% yield. LC/MS (m/z): 371.3 (MH⁺), Rₜ=0.82 min. The material was separated via chiral HPLC (IC column, heptane:EtOH 95:05) to give (2R,3R,4S,5S,6S)-6-(3-aminopyridin-4-yl)-4-(tert-butyldimethylsilyloxy)-5-fluoro-2,3-dimethyltetrahydro-2H-pyran-3-ol (>99% ee) and (2S,3S,4R,5R,6R)-6-(3-aminopyridin-4-yl)-4-(tert-butyldimethylsilyloxy)-5-fluoro-2,3-dimethyltetrahydro-2H-pyran-3-ol (>99% ee).

### Synthesis of (+/−)-3-nitro-4-(7-(trimethylsilyloxy)-4-oxaspiro[2.5]oct-7-en-5-yl)pyridine and (+/−)-5-(3-nitropyridin-4-yl)-4-oxaspiro[2.5]octan-7-one

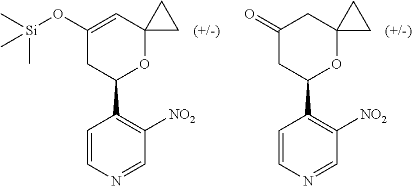 [SMILES: C[Si](C)(C)OC1=CC2(CC2)O[C@@H](c2ccncc2[N+](=O)[O-])C1 ; O=C1C[C@H](c2ccncc2[N+](=O)[O-])OC2(CC2)C1]

(3-cyclopropylideneprop-1-en-2-yloxy)trimethylsilane (1.5 equiv.), 3-nitroisonicotinaldehyde (1.0 equiv.) and tris(6,6,7,7,8,8,8-heptafluoro-2,2-dimethyl-3,5-octanedionato) europium (0.05 equiv.) were dissolved in CHCl₃ (1.4 M) in a flame dried rbf and stirred at 60° C. under Argon for 2 hours. Upon cooling, the volatiles were removed in vacuo and the material was purified by ISCO SiO₂ chromatography eluting with 0-80% EtOAc in Heptanes to give (+/−)-3-nitro-4-(7-(trimethylsilyloxy)-4-oxaspiro[2.5]oct-7-en-5-yl)pyridine in 27% yield along with (+/−)-5-(3-nitropyridin-4-yl)-4-oxaspiro[2.5]octan-7-one in 39% yield. For silyl enol ether product, ¹H NMR (400 MHz, CDCl₃-d) δ ppm 9.33 (s, 1H), 8.97 (d, 1H), 7.80 (d, 1H), 5.42 (dd, 1H), 4.62 (d, 1H), 2.58 (dd, 1H), 2.30 (ddd, 1H), 1.16-1.22 (m, 1H), 0.85-0.91 (m, 1H), 0.70-0.75 (m, 1H), 0.58-0.63 (m, 1H). For ketone product, LC/MS (m/z): 249.1 (MH⁺), Rₜ=0.66 min. ¹H NMR (400 MHz, CDCl₃-d) δ ppm 9.20 (s, 1H), 8.86 (d, 1H), 7.80 (d, 1H), 5.42 (dd, 1H), 3.20 (d, 1H), 3.00 (ddd, 1H), 2.45 (dd, 1H), 1.99 (dd, 1H), 1.08-1.14 (m, 1H), 0.80-0.84 (m, 1H), 0.67-0.84 (m, 1H), 0.57-0.61 (m, 1H).

### Synthesis of Cis (+/−)5-(3-nitropyridin-4-yl)-4-oxaspiro[2.5]octan-7-ol

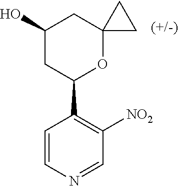 [SMILES: O=[N+]([O-])c1cnccc1[C@H]1C[C@@H](O)CC2(CC2)O1]

To a solution of (+/−)-5-(3-nitropyridin-4-yl)-4-oxaspiro[2.5]octan-7-one (1.0 equiv.) in MeOH (0.05 M) at 0° C. was added sodium borohydride (1.0 equiv.). After stirring in the ice bath for 10 minutes, water was added to quench and the volatiles were removed in vacuo. The residue was partitioned between EtOAc and NaCl_{(sat.)}, separated, dried over MgSO₄, filtered and the volatiles were removed in vacuo to yield Cis (+/−)5-(3-nitropyridin-4-yl)-4-oxaspiro[2.5]octan-7-ol in 90% yield. LC/MS (m/z): 251.1 (MH⁺), Rₜ=0.59 min.

### Synthesis of Cis (+/−)4-(7-(tert-butyldimethylsilyloxy)-4-oxaspiro[2.5]octan-5-yl)-3-nitropyridine

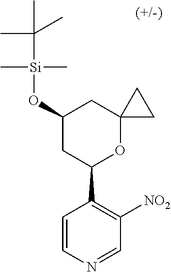 [SMILES: CC(C)(C)[Si](C)(C)O[C@@H]1C[C@H](c2ccncc2[N+](=O)[O-])OC2(CC2)C1]

(+/−)5-(3-nitropyridin-4-yl)-4-oxaspiro[2.5]octan-7-ol (1.0 equiv.) was dissolved in DMF (0.8 M). 1H-imidazole (5.0 equiv.) and tert-butylchlorodimethylsilane (2.0 equiv.) were added and the reaction stirred for 4 hours. The solution was poured into water and extracted with EtOAc, dried over sodium sulfate, filtered and concentrated. The residue was purified by ISCO using a RediSep column eluting with 10% EtOAc in Heptanes to give Cis (+/−)4-(7-(tert-butyldimethylsilyloxy)-4-oxaspiro[2.5]octan-5-yl)-3-nitropyridine in 69% yield. LC/MS (m/z): 365.3 (MH⁺), Rₜ=1.34 min. ¹H NMR (400 MHz, CHLOROFORM-d) δ ppm 0.10 (d, 6H) 0.44 (ddd, 1H) 0.56-0.63 (m, 1H) 0.67-0.75 (m, 1H) 0.89 (dt, 9H) 0.91-0.96 (m, 1H) 1.37 (dd, 1H) 1.41-1.51 (m, 1H) 2.17 (t, 1H) 2.32-2.39 (m, 1H) 4.09-4.19 (m, 1H) 5.08 (d, 1H) 7.75 (d, 1H) 8.79 (d, 1H) 9.17 (s, 1H).

### Synthesis of Cis (+/−)4-(7-(tert-butyldimethylsilyloxy)-4-oxaspiro[2.5]octan-5-yl)pyridin-3-amine

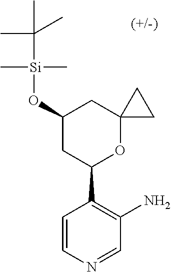 [SMILES: CC(C)(C)[Si](C)(C)O[C@@H]1C[C@H](c2ccncc2N)OC2(CC2)C1]

Cis (+/−)4-(7-(tert-butyldimethylsilyloxy)-4-oxaspiro[2.5]octan-5-yl)-3-nitropyridine (1.0 equiv.) was dissolved in AcOH (0.13 M) and Iron (5.0 equiv.) was added. The mixture was stirred vigorously for 3 hours. The mixture was filtered through celite eluting with EtOAc and then concentrated. The residue was partitioned between EtOAc and water and separated. The organics were washed with sat. sodium carbonate, brine, dried over sodium sulfate, filtered and concentrated to give Cis (+/−)4-(7-(tert-butyldimethylsilyloxy)-4-oxaspiro[2.5]octan-5-yl)-3-nitropyridine in 70% yield. LC/MS (m/z): 335.3 (MH⁺), Rₜ=0.91 min.

### Synthesis of (±)-(5R)-8-((dimethylamino)methyl)-5-(3-nitropyridin-4-yl)-4-oxaspiro[2.5]octan-7-one

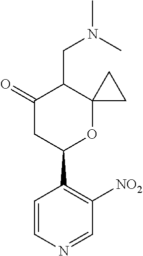 [SMILES: CN(C)CC1C(=O)C[C@H](c2ccncc2[N+](=O)[O-])OC12CC2]

A solution of N-methyl-N-methylenemethanaminium iodide (2 equiv.) and (+/−)-3-nitro-4-(7-(trimethylsilyloxy)-4-oxaspiro[2.5]oct-7-en-5-yl)pyridine (1.0 equiv.) in DCM (0.4 M) at room temperature was stirred for 3 days. Aqueous 1N HCl (2 equiv.) was added into the reaction mixture, and after stirring at room temperature for 1 h, the reaction mixture was basified to pH=14 by addition of 3 N NaOH solution. The reaction mixture was then extracted by EtOAc, the organic was washed by water and brine, dried over anhydrous sodium sulfate, filtered and concentrated in vacuo to give (±)-(5R)-8-((dimethylamino)methyl)-5-(3-nitropyridin-4-yl)-4-oxaspiro[2.5]octan-7-one in 90% yield. LCMS (m/z): 306.1 (MH⁺)/324.1 (M+H₃O⁺), Rₜ=0.45 min.

### Synthesis of (±)-8-methylene-5-(3-nitropyridin-4-yl)-4-oxaspiro[2.5]octan-7-one

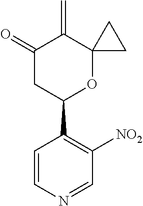 [SMILES: C=C1C(=O)C[C@H](c2ccncc2[N+](=O)[O-])OC12CC2]

To a solution of (±)-8-((dimethylamino)methyl)-5-(3-nitropyridin-4-yl)-4-oxaspiro[2.5]octan-7-one (1.0 equiv.) in CHCl₃ (0.25 M) was added MeI (5 equiv) at rt. The reaction mixture stirred at room temperature for 26 hours, at which time additional MeI (1.0 equiv) was added and the solution stirred for an additional 7 hours. The volatiles were removed in vacuo and the residue was dissolved in 1:1 THF/H₂O (0.1 M), cooled to 0° C. and NaHCO₃ (5 equiv.) was added. After stirring for 2.5 hours, the solution was partitioned between hexanes and NaCl_{(sat.)}, separated. The aqueous was extracted further with hexanes (100 mL) and the combined organics were dried over MgSO₄, filtered, concentrated to yield crude (±)-(R)-8-methylene-5-(3-nitropyridin-4-yl)-4-oxaspiro[2.5]octan-7-one. LC/MS (m/z): 261.0 (MH⁺), Rₜ=0.73 min.

### Synthesis of (±)-8-methylene-5-(3-nitropyridin-4-yl)-4-oxaspiro[2.5]octan-7-ol

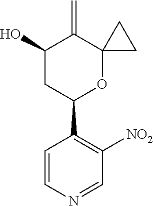 [SMILES: C=C1[C@H](O)C[C@H](c2ccncc2[N+](=O)[O-])OC12CC2]

To a solution of (±)-8-methylene-5-(3-nitropyridin-4-yl)-4-oxaspiro[2.5]octan-7-one in methanol (0.1M) at 0° C. was added cerium(III) chloride heptahydrate (1.2 equiv) and than NaBH₄ (1.2 equiv). After stirring for 5 minutes, the reaction was quenched with H₂O and the volatiles were removed in vacuo. The residue was partitioned between EtOAc and NaCl_{(sat.)}, separated, dried over MgSO₄, filtered and the volatiles were removed in vacuo to yield cis-(±)-8-methylene-5-(3-nitropyridin-4-yl)-4-oxaspiro[2.5]octan-7-ol in 34% yield (from (±)-8-((dimethylamino)methyl)-5-(3-nitropyridin-4-yl)-4-oxaspiro[2.5]octan-7-one). LC/MS (m/z): 263.1 (MH⁺) Rₜ=0.67 min.

### Synthesis of cis (+/−)-4-(7-(tert-butyldimethylsilyloxy)-8-methylene-4-oxaspiro[2.5]octan-5-yl)-3-nitropyridine

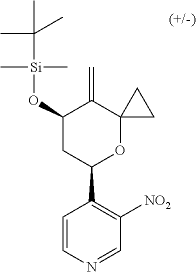 [SMILES: C=C1[C@H](O[Si](C)(C)C(C)(C)C)C[C@H](c2ccncc2[N+](=O)[O-])OC12CC2]

To a solution of cis-(±)-8-methylene-5-(3-nitropyridin-4-yl)-4-oxaspiro[2.5]octan-7-ol (1.0 equiv.) in DMF (0.8 M) was added 1H-imidazole (5.0 equiv.) and tert-butylchlorodimethylsilane (2.0 equiv.). After stirring for 54 hours, the solution was poured into water, extracted with EtOAc, dried over sodium sulfate, filtered and concentrated. The residue was purified by ISCO using a RediSep column eluting with 10% EtOAc in Heptanes to give cis (+/−)-4-(7-(tert-butyldimethylsilyloxy)-8-methylene-4-oxaspiro[2.5]octan-5-yl)-3-nitropyridine in 85% yield. LC/MS (m/z): 377.2 (MH⁺) Rₜ=1.38 min.

### Synthesis of cis (+/−)-4-(7-(tert-butyldimethylsilyloxy)-8-methylene-4-oxaspiro[2.5]octan-5-yl)pyridin-3-amine

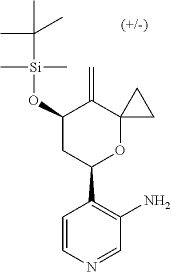 [SMILES: C=C1[C@H](O[Si](C)(C)C(C)(C)C)C[C@H](c2ccncc2N)OC12CC2]

To a solution of (+/−)-4-(7-(tert-butyldimethylsilyloxy)-8-methylene-4-oxaspiro[2.5]octan-5-yl)-3-nitropyridine (1.0 equiv.) in AcOH (0.13 M) was added Iron (5.0 equiv.). The mixture was stirred vigorously for 3 hours. The mixture was filtered through celite eluting with EtOAc and then concentrated. The residue was partitioned between EtOAc and water and separated. The organics were washed with sat. sodium carbonate, brine, dried over sodium sulfate, filtered and concentrated to give cis (+/−)-4-(7-(tert-butyldimethylsilyloxy)-8-methylene-4-oxaspiro[2.5]octan-5-yl)pyridin-3-amine in 87% yield. LC/MS (m/z): 347.1 (MH⁺), Rₜ=0.99 min. The material was separated via chiral HPLC (IC column, heptane:IPA 80:20, Rₜ's=3.87 and 5.42 min).

### Synthesis of (+/−)-3-(tert-butyldimethylsilyloxy)-2,3-dimethyl-6-(3-nitropyridin-4-yl)dihydro-2H-pyran-4(3H)-one

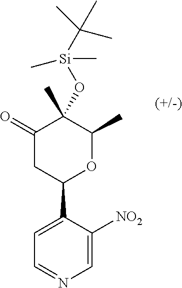 [SMILES: C[C@H]1O[C@@H](c2ccncc2[N+](=O)[O-])CC(=O)[C@]1(C)O[Si](C)(C)C(C)(C)C]

(+/−)3-hydroxy-2,3-dimethyl-6-(3-nitropyridin-4-yl)dihydro-2H-pyran-4(3H)-one (1.0 equiv.) was dissolved in dry DCM (0.2 M) under N₂ and cooled to 0° C. on an ice bath. 2,6-dimethylpyridine (4.0 equiv.) was added followed by tert-butyldimethylsilyl trifluoromethanesulfonate (3.0 equiv.). The reaction was stirred at 0° C. allowing to warm to room temperature for 17 hours. The solution was poured into sat. sodium bicarbonate and EtOAc was added. The layers were separated and the EtOAc layer was washed with 10% aqueous copper sulfate, brine, dried over sodium sulfate, filtered and concentrated. The residue was purified by ISCO using a g column eluting with 0-70% EtOAc in Heptanes give (+/−)3-(tert-butyldimethylsilyloxy)-2,3-dimethyl-6-(3-nitropyridin-4-yl)dihydro-2H-pyran-4(3H)-one in 70% yield. LC/MS (m/z): 381.1 (MH⁺), Rₜ=1.30 min. ¹H NMR (400 MHz, CDCl₃-d) δ ppm 9.20 (s, 1H), 8.88 (d, 1H), 7.84 (d, 1H), 5.33 (dd, 1H), 3.72 (q, 1H), 2.96 (dd, 1H), 2.60 (dd, 1H), 1.36 (d, 3H) 1.43 (s, 3H), 0.89 (s, 9H), 0.21 (s, 3H), 0.16 (s, 3H).

### Synthesis of (+/−)3-(tert-butyldimethylsilyloxy)-2,3-dimethyl-6-(3-nitropyridin-4-yl)-5-(phenylselanyl)dihydro-2H-pyran-4(3H)-one

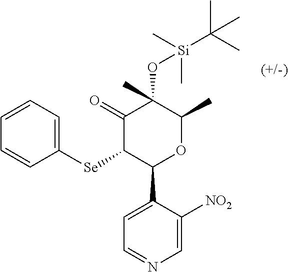 [SMILES: C[C@H]1O[C@@H](c2ccncc2[N+](=O)[O-])[C@H]([Se]c2ccccc2)C(=O)[C@]1(C)O[Si](C)(C)C(C)(C)C]

To a solution of LiHMDS (1.0 M in THF) (1.5 equiv.) in a flame dried rbf under nitrogen was added a solution of (+/−)3-(tert-butyldimethylsilyloxy)-2,3-dimethyl-6-(3-nitropyridin-4-yl)dihydro-2H-pyran-4(3H)-one (1.0 equiv.) in THF (0.14 M) at −78° C. over 30 min. After stirring an additional hour at −78° C. a solution of phenylselenyl bromide (1.5 equiv.) in THF (0.5 M) was added drop wise. The reaction was stirred at −78° C. for 1 hour and then water was added. The mixture was extracted with EtOAc, washed with brine, dried over sodium sulfate, and concentrated. The residue was purified by ISCO using a RediSep column eluting with 0-100% EtOAc in Hexanes to give (+/−)3-(tert-butyldimethylsilyloxy)-2,3-dimethyl-6-(3-nitropyridin-4-yl)-5-(phenylselanyl)dihydro-2H-pyran-4(3H)-one in 24% yield. LC/MS (m/z): 535.0 and 537.0 (MH⁺), Rₜ=0.96 min. ¹H NMR (400 MHz, CDCL₃-d) δ ppm 0.18 (s, 3H) 0.25 (s, 3H) 0.90 (s, 9H) 1.30 (d, 3H) 1.54 (s, 3H) 3.70 (q, 1H) 4.63 (d, 1H) 5.25 (d, 1H) 6.96-7.02 (m, 2H) 7.06-7.13 (m, 3H) 7.32 (d, 1H) 8.45 (d, 1H) 8.81 (s, 1H).

### Synthesis of (+/−)3-(tert-butyldimethylsilyloxy)-2,3-dimethyl-6-(3-nitropyridin-4-yl)-2H-pyran-4(3H)-one

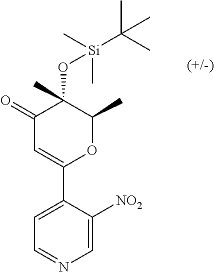 [SMILES: C[C@H]1OC(c2ccncc2[N+](=O)[O-])=CC(=O)[C@]1(C)O[Si](C)(C)C(C)(C)C]

(+/−)3-(tert-butyldimethylsilyloxy)-2,3-dimethyl-6-(3-nitropyridin-4-yl)-5-(phenylselanyl)dihydro-2H-pyran-4(3H)-one was dissolved in THF/Water (4:1) (0.1 M) and sodium periodate (4.0 equiv.) was added in one portion. The reaction was stirred for 5 hours. Sodium thiosulfate (1 M) was added and the mixture was diluted with water and extracted with EtOAc, dried over sodium sulfate and concentrated. This material was purified by ISCO using a RediSep column eluting with 0-50% EtOAc in Hexanes to give (+/−)3-(tert-butyldimethylsilyloxy)-2,3-dimethyl-6-(3-nitropyridin-4-yl)-2H-pyran-4(3H)-one in 76% yield. LC/MS (m/z): 379.1 (MH⁺), Rₜ=1.34 min. ¹H NMR (400 MHz, CHLOROFORM-d) δ ppm 0.15 (s, 3H) 0.24 (s, 3H) 0.87 (s, 9H) 1.30 (s, 3H) 1.40 (d, 3H) 4.42 (q, 1H) 5.72 (s, 1H) 7.47 (d, 1H) 8.90 (d, 1H) 9.14 (s, 1H).

### Synthesis of (+/−)3-hydroxy-2,3-dimethyl-6-(3-nitropyridin-4-yl)-2H-pyran-4(3H)-one

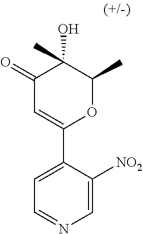 [SMILES: C[C@H]1OC(c2ccncc2[N+](=O)[O-])=CC(=O)[C@]1(C)O]

(+/−)3-(tert-butyldimethylsilyloxy)-2,3-dimethyl-6-(3-nitropyridin-4-yl)-2H-pyran-4(3H)-one was dissolved in THF (0.2 M) and HCl (6 M) (10.0 equiv.) was added. The reaction was heated to 60° C. for 4 hours. The solvents were removed in vacuo and the residue partitioned between EtOAc and sat. sodium bicarbonate. The aqueous was extracted with EtOAc, the combined organics were washed with brine, dried over sodium sulfate, filtered and concentrated. The crude residue was purified by ISCO using a RediSep column eluting with 0-100% EtOAc in Hexanes to give (+/−)3-hydroxy-2,3-dimethyl-6-(3-nitropyridin-4-yl)-2H-pyran-4(3H)-one in 79% yield. LC/MS (m/z): 265.0 (MH⁺), Rₜ=0.59 min. ¹H NMR (400 MHz, CDCl₃-d) δ ppm 9.18 (s, 1H), 8.93 (d, 1H), 7.48 (d, 1H), 5.81 (s, 1H), 4.45 (q, 1H), 3.66 (s, 1H), 1.44 (d, 3H), 1.31 (s, 3H).

### Synthesis of (+/−)2,3-dimethyl-6-(3-nitropyridin-4-yl)-3,4-dihydro-2H-pyran-3,4-diol

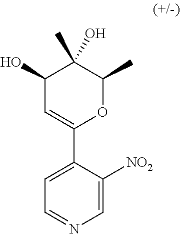 [SMILES: C[C@H]1OC(c2ccncc2[N+](=O)[O-])=C[C@@H](O)[C@]1(C)O]

3-hydroxy-2,3-dimethyl-6-(3-nitropyridin-4-yl)-2H-pyran-4(3H)-one (1 equiv.) was dissolved in EtOH (0.1 M) and cerium(III) chloride heptahydrate (1.2 equiv.) was added and the mixture was stirred for 10 min. Sodium tetrahydroborate (1.2 equiv.) was added and the reaction stirred at room temperature for 30 min. and then quenched with water. The mixture was extracted with EtOAc, dried over sodium sulfate, decanted and concentrated to give (+/−)2,3-dimethyl-6-(3-nitropyridin-4-yl)-3,4-dihydro-2H-pyran-3,4-diol in quantitative yield. LC/MS (m/z): 267.1 (MH⁺), Rₜ=0.50 min. ¹H NMR (400 MHz, CHLOROFORM-d) δ ppm 1.25 (s, 3H) 1.33 (d, 3H) 4.04-4.18 (m, 2H) 4.44 (br. s., 1H) 5.27 (d, 1H) 7.42 (d, 1H) 8.74 (d, 1H) 8.95 (s, 1H).

### Synthesis of (+/−)4-(tert-butyldimethylsilyloxy)-2,3-dimethyl-6-(3-nitropyridin-4-yl)-3,4-dihydro-2H-pyran-3-ol

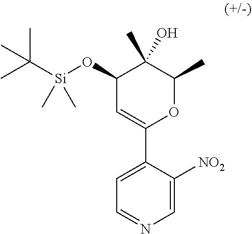 [SMILES: C[C@H]1OC(c2ccncc2[N+](=O)[O-])=C[C@@H](O[Si](C)(C)C(C)(C)C)[C@]1(C)O]

(+/−)2,3-dimethyl-6-(3-nitropyridin-4-yl)-3,4-dihydro-2H-pyran-3,4-diol) (1.0 equiv.) was mixed with 1H-imidazole (5.0 equiv.) and tert-butylchlorodimethylsilane (2.0 equiv.) DMF (0.8 M) was added and the reaction stirred for 16 hours. The solution was poured into water and extracted with EtOAc, dried over sodium sulfate, filtered and concentrated. The residue was purified by ISCO using a RediSep column eluting with 10% EtOAc in Heptanes to give to give (+/−)4-(tert-butyldimethylsilyloxy)-2,3-dimethyl-6-(3-nitropyridin-4-yl)-3,4-dihydro-2H-pyran-3-ol in 86% yield. LC/MS (m/z): 381.0 (MH⁺), Rₜ=1.12 min.

### Synthesis of (+/−)6-(3-aminopyridin-4-yl)-4-(tert-butyldimethylsilyloxy)-2,3-dimethyl-3,4-dihydro-2H-pyran-3-ol

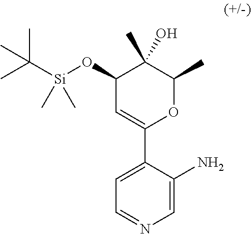 [SMILES: C[C@H]1OC(c2ccncc2N)=C[C@@H](O[Si](C)(C)C(C)(C)C)[C@]1(C)O]

(+/−) (4-(tert-butyldimethylsilyloxy)-2,3-dimethyl-6-(3-nitropyridin-4-yl)-3,4-dihydro-2H-pyran-3-ol (1.0 equiv.) was dissolved in AcOH (0.13 M) and Iron (5.0 equiv.) was added. The mixture was stirred vigorously for 3 hours. The mixture was concentrated and partitioned between EtOAc and water. The organics were washed with sat. sodium carbonate, brine, dried over sodium sulfate, filtered and concentrated to give (+/−)6-(3-aminopyridin-4-yl)-4-(tert-butyldimethylsilyloxy)-2,3-dimethyl-3,4-dihydro-2H-pyran-3-ol 78% yield. LC/MS (m/z): 351.1 (MH⁺), Rₜ=0.80 min.

### Synthesis of ((2R,3R,4R,6R)-6-(3-aminopyridin-4-yl)-3,4-bis(triisopropylsilyloxy)tetrahydro-2H-pyran-2-yl)methanol

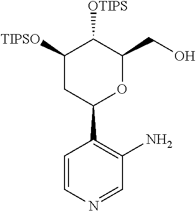

To a solution of 4-((2S,4R,5R,6R)-4,5-bis(triisopropylsilyloxy)-6-((triisopropylsilyloxy)methyl)tetrahydro-2H-pyran-2-yl)pyridin-3-amine (1.0 equiv.) in THF at 0° C. was added concentrated HCl (5.0 equiv.) dropwise. The reaction was allowed to warm to room temperature and stirred for 4 h. Another 5 equiv. of HCl was added at room temperature and stirred for another 1 h. The reaction was then carefully neutralized by slow addition of sat. NaHCO₃, the solution was extracted with ethyl acetate, dried with sodium sulfate, filtered and concentrated. The crude material was triturated in ethyl acetate and the precipitate was filtered off. The filtrate was concentrated and purified via silica gel column chromatography eluting with 0-100% ethyl acetate to afford ((2R,3R,4R,6R)-6-(3-aminopyridin-4-yl)-3,4-bis(triisopropylsilyloxy)tetrahydro-2H-pyran-2-yl)methanol as a white solid in 40% combined yield. LC/MS (m/z): 553.2 (MH⁺) Rₜ=0.29 min (6595 method).

### Synthesis of 4-((2R,4R,5R,6S)-6-(iodomethyl)-4,5-bis(triisopropylsilyloxy)tetrahydro-2H-pyran-2-yl)pyridin-3-amine

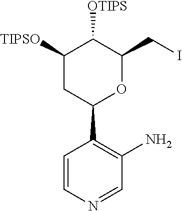

To a solution of ((2R,3R,4R,6R)-6-(3-aminopyridin-4-yl)-3,4-bis(triisopropylsilyloxy)tetrahydro-2H-pyran-2-yl)methanol (1.0 equiv.) in benzene (0.07 M) was added imidazole (1.5 equiv.), followed by triphenyl phosphine (1.5 equiv.) and iodine (1.3 equiv.). The reaction turned brown and it was stirred at room temperature for 2 h. Another 0.5 equiv. of imidazole, triphenyl phosphine and iodine were added and stirred for another 3 h. Upon completion of the reaction, quenched with sat. Na₂SO₃, extracted with ethyl acetate, dried with sodium sulfate, filtered and concentrated. The crude material was purified via silica gel column chromatography eluting with ethyl acetate and hexanes (0-50% ethyl acetate) to give 4-((2R,4R,5R,6S)-6-(iodomethyl)-4,5-bis(triisopropylsilyloxy)tetrahydro-2H-pyran-2-yl)pyridin-3-amine as a white foam in 82% yield. LC/MS (m/z): 663.3 (MH⁺) Rₜ=1.18 min (6595 method).

### Synthesis of 2-((2R,3R,4R,6R)-6-(3-aminopyridin-4-yl)-3,4-bis(triisopropylsilyloxy)tetrahydro-2H-pyran-2-yl)acetonitrile

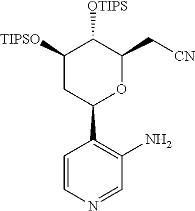

To a solution of 4-((2R,4R,5R,6S)-6-(iodomethyl)-4,5-bis(triisopropylsilyloxy)tetrahydro-2H-pyran-2-yl)pyridin-3-amine (1.0 equiv.) in DMSO (0.06 M) was added KCN (5 equiv.) and the reaction was stirred at room temperature overnight. The solution was partitioned between water and ethyl acetate. The aqueous phase was extracted with ethyl acetate three times, the organics were combined, washed with sat. NaCl, dried with sodium sulfate, filtered and concentrated to give 2-((2R,3R,4R,6R)-6-(3-aminopyridin-4-yl)-3,4-bis(triisopropylsilyloxy)tetrahydro-2H-pyran-2-yl)acetonitrile as the desired product in 96% yield. LC/MS (m/z): 562.4 (MH⁺) Rₜ=0.92 min (6595 method).

### Synthesis of N-(4-((2R,4R,5R,6R)-6-(cyanomethyl)-4,5-bis(triisopropylsilyloxy)tetrahydro-2H-pyran-2-yl)pyridin-3-yl)-6-(2,6-difluorophenyl)-5-fluoropicolinamide

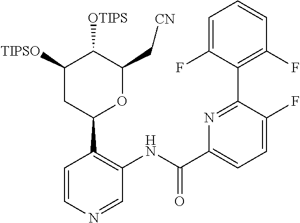

To a solution of 2-((2R,3R,4R,6R)-6-(3-aminopyridin-4-yl)-3,4-bis(triisopropylsilyloxy)tetrahydro-2H-pyran-2-yl)acetonitrile (1.0 equiv.) in DMF (0.14M) was added 6-(2,6-difluorophenyl)-5-fluoropicolinic acid (1.5 equiv.), EDCI (1.5 equiv.) and HOAt (1.5 equiv.) The reaction was stirred at room temperature for 2 days. Water was added and the precipitate was filtered off to give N-(4-((2R,4R,5R,6R)-6-(cyanomethyl)-4,5-bis(triisopropylsilyloxy)tetrahydro-2H-pyran-2-yl)pyridin-3-yl)-6-(2,6-difluorophenyl)-5-fluoropicolinamide as a white solid in 73% yield. LC/MS (m/z): 797.4 (MH⁺) Rₜ=1.25 min (6595 method).

### Synthesis of N-(4-((2R,4R,5S,6R)-6-(2-amino-2-oxoethyl)-4,5-dihydroxytetrahydro-2H-pyran-2-yl)pyridin-3-yl)-6-(2,6-difluorophenyl)-5-fluoropicolinamide

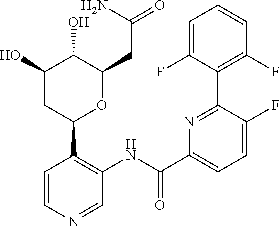 [SMILES: NC(=O)C[C@H]1O[C@@H](c2ccncc2NC(=O)c2ccc(F)c(-c3c(F)cccc3F)n2)C[C@@H](O)[C@@H]1O]

A solution of N-(4-((2R,4R,5R,6R)-6-(cyanomethyl)-4,5-bis(triisopropylsilyloxy)tetrahydro-2H-pyran-2-yl)pyridin-3-yl)-6-(2,6-difluorophenyl)-5-fluoropicolinamide (1.0 equiv.) was dissolved in 33% HBr in AcOH (0.04M). The reaction was stirred at room temperature for 4 h. The acetylated product was poured in ice water and extracted with chloroform. The aqueous phase was basified with NaOH and extracted with chloroform two more times. The organics were combined, dried with sodium sulfate, filtered and concentrated. The crude material was stirred in EtOH and potassium carbonate (5 equiv.) and heated to 60° C. Upon completion of the deprotection, the reaction was quenched by the addition of water, the volatiles were removed under vacuo, the solution was partitioned between ethyl acetate and water, the organic phase was dried with sodium sulfate, filtered and concentrated. The crude material was purified via reverse phase HPLC and the pure fractions were lyophilized for several days to give N-(4-((2R,4R,5S,6R)-6-(2-amino-2-oxoethyl)-4,5-dihydroxytetrahydro-2H-pyran-2-yl)pyridin-3-yl)-6-(2,6-difluorophenyl)-5-fluoropicolinamide as a white fluffy powder. LC/MS (m/z): 503.1 (MH⁺) Rₜ=0.52 min.

### Synthesis of (E)-3-methylhex-3-en-2-one

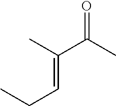

To a solution of (E)-2-methyl-2-pentenoic acid (1.0 equiv.) in THF (0.08 M) cooled to −78° C. was added rapidly via syringe MeLi (1.6 M in Et₂₀, 1.0 equiv.). The resulting mixture was stirred at −78° C. for 1 h before the reaction mixture was warmed to 0° C. (dry-ice acetone bath was replaced with an ice/water bath) and stirred for a further 1 h. The reaction mixture was quenched by cannula transfer into a solution of 0.12 M HCl (150 ml). The organic phase was then separated and washed successively with aq.sat. Na₂CO₃ (50 ml, ×²) followed by brine (50 ml). The organic layer was then dried over MgSO₄, filtered and concentrated by atmospheric distillation to remove the volatile solvents. The volume was reduced to approximately 5 ml and transferred to a bulb to bulb distillation apparatus. The crude oil was further purified by bulb to bulb distillation at atmospheric pressure to afford the desired product (E)-3-methylhex-3-en-2-one as a pale yellow oil (yield=73%). LC/MS (m/z): 112.8 (MH⁺), Rₜ=0.78 min. ¹H NMR (400 MHz, CHLOROFORM-d) δ ppm 1.09 (t, 3H, J=7.6 Hz) 1.76 (s, 3H), 2.19-2.26 (m, 2H), 2.31 (s, 3H), 6.62 (t, 1H, J=6.4 Hz).

### Synthesis of (E)-triethyl((3-methylhexa-1,3-dien-2-yl)oxy)silane

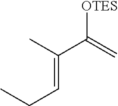

To a solution of (E)-3-methylhex-3-en-2-one (1.0 equiv.) and triethylamine (1.2 equiv.) in Et₂O (0.248 M) cooled to 0° C. was added triethylsilyl trifluoromethanesulfonate (1.1 equiv.) dropwise over five minutes. The resulting mixture was stirred at 0° C. for 2 h. The reaction mixture was then quenched with NaHCO₃ (10 ml), the aqueous layer was separated and extracted with Et₂O (10 ml). The combined organics were then dried over MgSO₄, filtered and concentrated in vacuo to yield the desired product (E)-triethyl((3-methylhexa-1,3-dien-2-yl)oxy)silane as a colourless oil (yield=99%) which was used in the Hetero-Diels Alder reaction without further purification.

### Synthesis of cis (+/−)-4-((2R,6R)-6-ethyl-5-methyl-4-((triethylsilyl)oxy)-3,6-dihydro-2H-pyran-2-yl)-3-nitropyridine

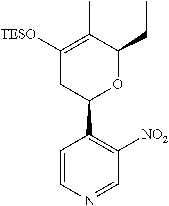

A solution of 3-nitroisonicotinaldehyde (1.5 equiv.), (E)-triethyl((3-methylhexa-1,3-dien-2-yl)oxy)silane (1.0 equiv.), and tris(6,6,7,7,8,8,8-heptafluoro-2,2-dimethyl-3,5-octanedionato) europium (0.05 equiv.) were dissolved in CHCl₃ (0.163 M) and stirred in a flame-dried round-bottom flask at 60° C. under an atmosphere of nitrogen for 4 hrs. After this time the reaction mixture was cooled to room temperature and concentrated in vacuo to yield a yellow oil. The oil was further purified by flash column chromatography by ISCO Combi-flash Rf system with a Redisep column eluting with 0-5% EtOAc/heptanes to afford the desired product cis (+/−)-4-((2R,6R)-6-ethyl-5-methyl-4-((triethylsilyl)oxy)-3,6-dihydro-2H-pyran-2-yl)-3-nitropyridine as a colourless oil (57% yield). LC/MS (m/z): 379.1 (MH⁺), Rₜ=1.01 min. ¹H NMR (400 MHz, CHLOROFORM-d) δ ppm 0.63-0.72 (m, 6H), 0.92-1.03 (m, 9H), 1.60 (m, 3H) overlapping with 1.54-1.64 (m, 1H), 1.78-1.90 (m, 1H), 2.00 (s, 3H), 2.20-2.31 (m, 1H), 2.46-2.54 (m, 1H), 4.21 (broad s, 1H), 5.22 (dd, 1H), 7.85 (d, 1H) 9.02 (d, 1H) 9.34 (s, 1H).

### Synthesis of (+/−)-(2R,3R,6S)-2-ethyl-3-hydroxy-3-methyl-6-(3-nitropyridin-4-yl)dihydro-2H-pyran-4(3H)-one

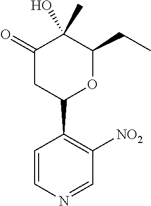

To a solution of cis-(+/−)-4-((2R,6R)-6-ethyl-5-methyl-4-((triethylsilyl)oxy)-3,6-dihydro-2H-pyran-2-yl)-3-nitropyridine (1.0 equiv.) in DCM (0.3 M) cooled to 0° C. was added 3,3-dimethyldioxirane as a solution in acetone (0.1M solution, 1.17 equiv.) and allowed to stir for 30 mins. To the reaction was added 10 mL of cyclohexene; the reaction mixture was stirred for 10 mins and the volatiles were removed in vacuo. The residue was taken up in THF (0.05 M) at room temperature and acidified with 5 mL of 1 M HCl (5.0 equiv.) the reaction was stirred for 15 min. The solution was basified with 2 M NaOH to ˜pH=9. The product was extracted in EtOAc, dried over MgSO₄, filtered and the volatiles were removed in vacuo. The oil was further purified by flash column chromatography by ISCO Combi-flash Rf system with a Redisep column eluting with 0-40% EtOAc/heptanes to afford as a single diastereoisomer the desired product (+/−)-(2R,3R,6S)-2-ethyl-3-hydroxy-3-methyl-6-(3-nitropyridin-4-yl)dihydro-2H-Pyran-4(3H)-one as a colourless oil (58% yield). LC/MS (m/z): 281.0 (MH⁺), Rₜ=0.68 min. ¹H NMR (400 MHz, CHLOROFORM-d) δ ppm 1.02 (t, 3H) 1.42 (s, 3H) 1.63-1.76 (m, 1H) 1.81-1.91 (m, 1H) 2.72 (dd, 1H) 3.06 (dd, 1H). 3.35 (dd, 1H), 3.85 (s, 1H), 5.33 (dd, 1H), 7.85 (d, 1H) 8.91 (d, 1H) 9.23 (s, 1H).

### Synthesis of (+/−)-(2R,3S,4R,6R)-2-ethyl-3-methyl-6-(3-nitropyridin-4-yl)tetrahydro-2H-pyran-3,4-diol

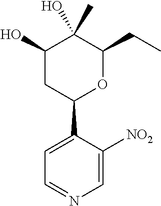 [SMILES: CC[C@H]1O[C@@H](c2ccncc2[N+](=O)[O-])C[C@@H](O)[C@]1(C)O]

To a solution of (+/−)-(2R,3R,6S)-2-ethyl-3-hydroxy-3-methyl-6-(3-nitropyridin-4-yl)dihydro-2H-pyran-4(3H)-one (1.0 equiv.) in EtOH (0.2 M) at 0° C. was added sodium borohydride (1.1 equiv.). The reaction mixture was allowed to stir for 30 min warming to room temperature. The reaction mixture was then concentrated and partitioned between water and EtOAc. The aqueous layer was then separated and extracted with EtOAc (×2) the combined organics were then washed with brine, dried over Na₂SO₄, filtered, and the volatiles were removed in vacuo to yield a mixture of C4 epimers (9:1 as determined by analytical UPLC). The oil was further purified by flash column chromatography by ISCO Combi-flash Rf system with a Redisep column eluting with 0-75% EtOAc/heptanes to afford as a single diastereoisomer the desired product (+/−)-(2R,3S,4R,6R)-2-ethyl-3-methyl-6-(3-nitropyridin-4-yl)tetrahydro-2H-pyran-3,4-diol as a colourless oil (93% yield). LC/MS (m/z): 283.0 (MH⁺), Rₜ=0.57 min. ¹H NMR (400 MHz, CHLOROFORM-d) δ ppm 1.01 (t, 3H) 1.23 (s, 3H) 1.44-1.57 (m, 2H) 1.71-1.86 (m, 1H), 2.33-2.43 (m, 1H), 3.18 (dd, 1H) 3.88 (dd, 1H), 5.16 (dd, 1H), 7.75 (d, 1H) 8.82 (d, 1H) 9.16 (s, 1H).

### Synthesis of (+/−)-(2R,3R,4R,6R)-2-ethyl-3-hydroxy-3-methyl-6-(3-nitropyridin-4-yl)tetrahydro-2H-pyran-4-yl acetate

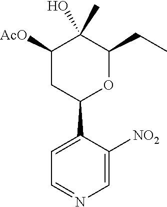 [SMILES: CC[C@H]1O[C@@H](c2ccncc2[N+](=O)[O-])C[C@@H](OC(C)=O)[C@]1(C)O]

To a solution of (+/−)-(2R,3S,4R,6R)-2-ethyl-3-methyl-6-(3-nitropyridin-4-yl)tetrahydro-2H-pyran-3,4-diol (1.0 equiv.) in pyridine (0.15 M) at room temperature was added acetic anhydride (5.0 equiv.). The reaction mixture was stirred for 19 hr at room temperature. The reaction was quenched with water and the product was extracted in EtOAc and washed with brine. The organics were dried over Na₂SO₄, filtered, and volatiles were removed in vacuo. The oil was further purified by flash column chromatography by ISCO Combi-flash Rf system with a Redisep column eluting with 0-50% EtOAc/heptanes to afford the desired product (+/−)-(2R,3R,4R,6R)-2-ethyl-3-hydroxy-3-methyl-6-(3-nitropyridin-4-yl)tetrahydro-2H-pyran-4-yl acetate as a colourless oil (76% yield). LC/MS (m/z): 324.9 (MH⁺), Rₜ=0.74 min. ¹H NMR (400 MHz, CHLOROFORM-d) δ ppm 0.98 (t, 3H) 1.23 (s, 3H) 1.42-1.56 (m, 1H), 1.58-1.71 (dd, 1H), 1.81-1.93 (m, 1H), 2.14 (s, 3H), 2.38-2.44 (m, 1H), 3.27 (dd, 1H), 5.06 (dd, 1H), 5.21 (dd, 1H), 7.75 (d, 1H) 8.84 (d, 1H) 9.18 (s, 1H).

### Synthesis of (2S,3S,4S,6S)-6-(3-aminopyridin-4-yl)-2-ethyl-3-hydroxy-3-methyltetrahydro-2H-pyran-4-yl acetate and (2R,3R,4R,6R)-6-(3-aminopyridin-4-yl)-2-ethyl-3-hydroxy-3-methyltetrahydro-2H-pyran-4-yl acetate

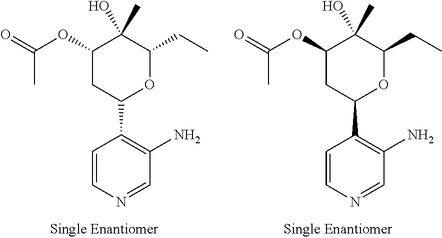

A solution of (+/−)-(2R,3R,4R,6R)-2-ethyl-3-hydroxy-3-methyl-6-(3-nitropyridin-4-yl)tetrahydro-2H-pyran-4-yl acetate (1.0 equiv.) in EtOH (0.183 M) was degassed with argon for 20 min. At room temperature under an Argon atmosphere, 10% Pd/C (20 mol %) was added and the resulting mixture was evacuated and backfilled with hydrogen gas (three times) and the mixture was then stirred at room temperature under atmospheric partial pressure of hydrogen gas (balloon) for 18 h. The reaction was filtered, and the volatiles were removed in vacuo. Purification was completed via chiral SFC (CO₂/EtOH+0.1% DEA=60/40, 15 mL/min, AD column) to yield in order of elution (2S,3S,4S,6S)-6-(3-aminopyridin-4-yl)-2-ethyl-3-hydroxy-3-methyltetrahydro-2H-pyran-4-yl acetate (20% yield, 99% ee) and (2R,3R,4R,6R)-6-(3-aminopyridin-4-yl)-2-ethyl-3-hydroxy-3-methyltetrahydro-2H-pyran-4-yl acetate (18% yield, 99% ee). LC/MS (m/z): 295.1 (MH⁺), Rₜ=0.43 min. ¹H NMR (400 MHz, CHLOROFORM-d) δ ppm 1.04 (t, 3H), 1.26 (s, 3H), 1.40-1.54 (m, 1H), 1.70 (broad s, 2H), 1.81-1.94 (m, 1H), 2.14 (s, 3H), 2.55 (broad s, 1H), 3.27 (dd, 1H), 4.23 (s, 2H), 4.56 (1H, dd), 4.98 (1H, dd), 6.94 (d, 1H) 7.98 (d, 1H) 8.02 (s, 1H).

### Synthesis of 3-nitro-4-((2R,3R,4R)-2-((E)-prop-1-en-1-yl)-3,4-bis((triisopropylsilyl)oxy)-3,4-dihydro-2H-pyran-6-yl)pyridine

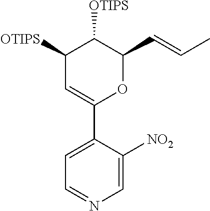

To a solution of ethyltriphenylphosphonium bromide (1.5 equiv.) in THF (0.173 M) cooled to −78° C. was added KHMDS (1.45 equiv.) dropwise. The resulting solution was stirred at −78° C. for 10 min before warming to 0° C. and stirred for a further 1 h resulting in the formation of a bright orange solution. The solution was then cooled to −78° C. and a solution of (2S,3R,4R)-6-(3-nitropyridin-4-yl)-3,4-bis((triisopropylsilyl)oxy)-3,4-dihydro-2H-pyran-2-carbaldehyde (1.0 equiv.) in THF (0.35 M) was added dropwise. The reaction mixture was allowed to warm to room temperature overnight. The reaction mixture was then quenched with a mixture of water and EtOAc then the organics were dried over Na₂SO₄, filtered, and concentrated in vacuo. The oil was further purified by flash column chromatography by ISCO Combi-flash Rf system with a Redisep column eluting with 0-25% EtOAc/heptanes to afford the desired product 3-nitro-4-((2R,3R,4R)-2-((E)-prop-1-en-1-yl)-3,4-bis((triisopropylsilyl)oxy)-3,4-dihydro-2H-pyran-6-yl)pyridine as a colourless oil (42% yield). LC/MS (m/z): 591.3 (MH⁺), Rₜ=1.26 min (65/95 method). ¹H NMR (400 MHz, CHLOROFORM-d) d ppm 1.02-1.10 (m, 42H), 1.70 (dd, 3H), 4.03 (d, 1H), 4.18-4.25 (m, 1H), 5.04-5.12 (m, 1H), 5.29-5.38 (m, 1H), 5.57-5.69 (m, 1H), 5.96 (ddd, 1H), 7.43 (d, 1H), 8.73 (d, 1H), 8.93 (s, 1H).

### Synthesis of 4-((2S,4R,5R,6R)-6-propyl-4,5-bis((triisopropylsilyl)oxy)tetrahydro-2H-pyran-2-yl)pyridin-3-amine

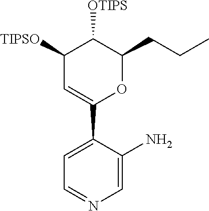

A solution of 3-nitro-4-((2R,3R,4R)-2-((E)-prop-1-en-1-yl)-3,4-bis((triisopropylsilyl)oxy)-3,4-dihydro-2H-pyran-6-yl)pyridine (1.0 equiv.) in EtOH (0.09 M) was degassed with argon for 20 min. At room temperature under an Argon atmosphere, 10% Pd/C (10 mol %) was added and the resulting mixture was evacuated and backfilled with hydrogen gas (three times) and the mixture was then stirred at room temperature under atmospheric partial pressure of hydrogen gas (balloon) for 16 h. The reaction was filtered, and the volatiles were removed in vacuo to afford the desired compound 4-((2S,4R,5R,6R)-6-propyl-4,5-bis((triisopropylsilyl)oxy)tetrahydro-2H-pyran-2-yl)pyridin-3-amine as a white solid (80% yield). LC/MS (m/z): 565.4 (MH⁺), Rₜ=1.28 min.

### Synthesis of ((1-(cyclohex-1-en-1-yl)vinyl)oxy)triethylsilane

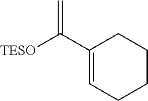

To a solution of LiHMDS (1.0 equiv.) in THF (0.5 M) cooled at −78° C. (internal thermometer) under N₂ was added a solution of 1-(cyclohex-1-en-1-yl)ethanone (1.0 equiv.) in THF (1.0 M) slowly over 50 min, keeping the internal temperature <−70° C. The resulting solution was stirred at −71° C. for 30 min before the dropwise addition of TES-Cl (1.10 equiv.) maintaining the internal temperature <−63° C. The cooling bath was then removed and the solution was allowed to warm to room temperature over 1.5 h. The reaction was poured into ice-cold saturated NaHCO₃ (400 mL) and Et₂O (1000 mL). The aqueous layer was separated and the organic layer was washed with NaHCO_{3(sat.)} (2×250 ml), brine then dried over Na₂SO₄, filtered and the volatiles were removed in vacuo to yield the desired product ((1-(cyclohex-1-en-1-yl)vinyl)oxy)triethylsilane as a colourless oil (99% yield). The oil was used without further purification. ¹H NMR (400 MHz, CHLOROFORM-d) δ ppm 0.72 (q, J=7.83 Hz, 6H) 1.00 (t, J=7.83 Hz, 9H) 1.54-1.71 (m, 4H) 2.11-2.17 (m, 4H) 4.19 (s, 1H) 4.33 (s, 1H) 6.24-6.27 (m, 1H)

### Synthesis of cis (+/−)-3-nitro-4-((2R,8aR)-4-((triethylsilyl)oxy)-3,5,6,7,8,8a-hexahydro-2H-chromen-2-yl)pyridine

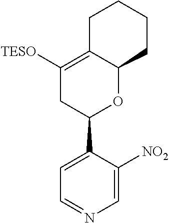

A solution of 3-nitroisonicotinaldehyde (1.0 equiv.), ((1-(cyclohex-1-en-1-yl)vinyl)oxy)triethylsilane (1.6 equiv.) and tris(6,6,7,7,8,8,8-heptafluoro-2,2-dimethyl-3,5-octanedionato) europium (0.05 equiv.) were dissolved in CHCl₃ (0.657 M) and stirred in a flame-dried round-bottom flask at 55° C. under an atmosphere of nitrogen for 1 hr. After this time the reaction mixture was cooled to room temperature and concentrated in vacuo to yield yellow oil. The oil was further purified by flash column chromatography by ISCO Combi-flash Rf system with a Redisep column eluting with 0-40% EtOAc/heptanes to afford the desired product cis (+/−)-3-nitro-4-((2R,8aR)-4-((triethylsilyl)oxy)-3,5,6,7,8,8a-hexahydro-2H-chromen-2-yl)pyridine as a colourless oil (97% yield). LC/MS (m/z): 391.1 (MH⁺), Rₜ=1.02 min (65/95 method).

¹H NMR (400 MHz, CHLOROFORM-d) ppm 0.67 (q, J=7.83 Hz, 6H) 0.95-1.01 (m, 9H) 1.29-1.40 (m, 2H) 1.52-1.65 (m, 2H) 1.73 (d, J=12.91 Hz, 1H) 1.78-1.85 (m, 1H) 2.20-2.31 (m, 2H) 2.43-2.53 (m, 1H) 2.89-2.97 (m, 1H) 4.09-4.16 (m, 1H) 5.20 (dd, J=10.56, 3.13 Hz, 1H) 7.83 (d, J=5.09 Hz, 1H) 8.85 (d, J=5.48 Hz, 1H) 9.18 (s, 1H)

### Synthesis of (+/−)-(2R,4aR,8aR)-4a-hydroxy-2-(3-nitropyridin-4-yl)hexahydro-2H-chromen-4(3H)-one

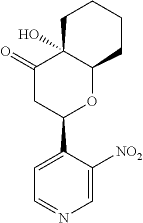 [SMILES: O=C1C[C@H](c2ccncc2[N+](=O)[O-])O[C@@H]2CCCC[C@]12O]

To a solution of cis-(+/−)-3-nitro-4-((2R,8aR)-4-((triethylsilyl)oxy)-3,5,6,7,8,8a-hexahydro-2H-chromen-2-yl)pyridine (1.0 equiv.) in DCM (0.2 M) cooled to 0° C. was added 3,3-dimethyldioxirane as a solution in acetone (0.1M solution, 1.00 equiv.) and allowed to stir for 2 hrs. To the reaction was added 5 mL of cyclohexene; the reaction mixture was stirred for 10 mins and the volatiles were removed in vacuo. The residue was taken up in THF (0.05 M) at room temperature and acidified with 5 mL of 1 M HCl (5.0 equiv.) the reaction stirred for 30 min. The solution was basified with 2 M NaOH to ˜pH=9. The product was extracted in EtOAc washed with brine, dried over MgSO₄, filtered and the volatiles were removed in vacuo. The oil was further purified by flash column chromatography by ISCO Combi-flash Rf system with a Redisep column eluting with 0-100% EtOAc/heptanes to afford as a single diastereoisomer the desired product (+/−)-(2R,4aR,8aR)-4a-hydroxy-2-(3-nitropyridin-4-yl)hexahydro-2H-chromen-4(3H)-one as a colourless oil (58% yield). LC/MS (m/z): 293.0 (MH⁺), Rₜ=0.68 min. ¹H NMR (400 MHz, CHLOROFORM-d) δ ppm 1.47-1.51 (m, 1H), 1.64-1.80 (m, 4H), 1.90-1.93 (m, 2H), 2.05-2.13 (m, 1H), 2.81 (dd, 1H), 3.03 (dd, 1H), 3.58 (m, 1H), 3.72 (s, 1H), 5.36 (dd, 1H), 7.89 (dd, 1H), 8.91 (dd, 1H), 9.22 (s, 1H).

### Synthesis of (+/−)-(2R,4R,4aS,8aR)-2-(3-nitropyridin-4-yl)octahydro-2H-chromene-4,4a-diol

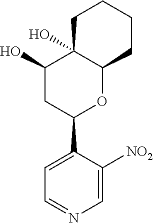 [SMILES: O=[N+]([O-])c1cnccc1[C@H]1C[C@@H](O)[C@@]2(O)CCCC[C@H]2O1]

To a solution of (+/−)-(2R,4aR,8aR)-4a-hydroxy-2-(3-nitropyridin-4-yl)hexahydro-2H-chromen-4(3H)-one (1.0 equiv.) in MeOH (0.135 M) at 0° C. was added sodium borohydride (1.0 equiv.). The reaction mixture was then stirred at 0° C. for 15 min. The reaction mixture was then quenched by the addition of water and stirred for 5 min before being concentrated in vacuo, the resulting residue was then partitioned between water and EtOAc. The aqueous layer was then separated and extracted with EtOAc (×2) the combined organics were then washed with brine, dried over MgSO₄, filtered, and the volatiles were removed in vacuo to yield the desired product as predominately a single diastereoisomer (+/−)-(2R,4R,4aS,8aR)-2-(3-nitropyridin-4-yl)octahydro-2H-chromene-4,4a-diol as a colourless oil (89% yield) as a white solid. LC/MS (m/z): 295.1 (MH⁺), Rₜ=0.57 min. The resulting solid was used in the subsequent transformation without further purification.

### Synthesis of (+/−)-(2R,4R,4aR,8aR)-4a-hydroxy-2-(3-nitropyridin-4-yl)octahydro-2H-chromen-4-yl acetate

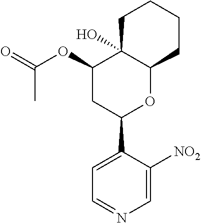 [SMILES: CC(=O)O[C@@H]1C[C@H](c2ccncc2[N+](=O)[O-])O[C@@H]2CCCC[C@]12O]

To a solution of (+/−)-(2R,4R,4aS,8aR)-2-(3-nitropyridin-4-yl)octahydro-2H-chromene-4,4a-diol (1.0 equiv.) in pyridine (0.15 M) at room temperature was added acetic anhydride (5.0 equiv.). The reaction mixture was stirred for 15 hr at room temperature after which time the mixture was concentrated in vacuo. The reaction was then pardoned between water and EtOAc. The organics were washed with CuSO₄ (10% aq.), brine then dried over MgSO₄, filtered, and volatiles were removed in vacuo. The residue was further purified by flash column chromatography by ISCO Combi-flash Rf system with a Redisep column eluting with 0-100% EtOAc/heptanes to afford the desired product (+/−)-(2R,3R,4R,6R)-2-ethyl-3-hydroxy-3-methyl-6-(3-nitropyridin-4-yl)tetrahydro-2H-pyran-4-yl acetate as a white solid (60% yield). LC/MS (m/z): 337.0 (MH⁺), Rₜ=0.76 min. ¹H NMR (400 MHz, CHLOROFORM-d) δ ppm 1.50-1.68 (m, 6H), 1.69-1.86 (m, 3H), 1.95-2.16 (m, 1H), 2.09 (s, 3H), 2.41 (m, 1H), 2.68 (broad s, 1H), 3.52 (m, 1H), 5.05 (dd, 1H), 5.20 (dd, 1H), 7.79 (d, 1H), 8.84 (d, 1H) 9.17 (s, 1H).

### Synthesis of (2S,4S,4aS,8aS)-2-(3-aminopyridin-4-yl)-4a-hydroxyoctahydro-2H-chromen-4-yl acetate and (2R,4R,4aR,8aR)-2-(3-aminopyridin-4-yl)-4a-hydroxyoctahydro-2H-chromen-4-yl acetate

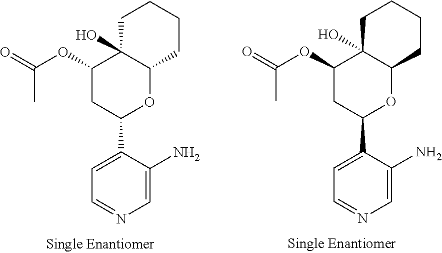 [SMILES: CC(=O)O[C@H]1C[C@@H](c2ccncc2N)O[C@H]2CCCC[C@@]12O ; CC(=O)O[C@@H]1C[C@H](c2ccncc2N)O[C@@H]2CCCC[C@]12O]

A solution of (+/−)-(2R,4R,4aR,8aR)-4a-hydroxy-2-(3-nitropyridin-4-yl)octahydro-2H-chromen-4-yl acetate (1.0 equiv.) in EtOH:EtOAc (1:1, 0.081 M) was degassed with argon for 20 min. At room temperature under an Argon atmosphere, 10% Pd/C (10 mol %) was added and the resulting mixture was evacuated and backfilled with hydrogen gas (three times) and the mixture was then stirred at room temperature under atmospheric partial pressure of hydrogen gas (balloon) for 5 h. The reaction was filtered, and the volatiles were removed in vacuo to yield a white solid. Purification was completed via chiral HPLC (EtOH/heptane)=40/60, 15 mL/min, AD column) to yield in order of elution (2S,3S,4S,6S)-6-(3-aminopyridin-4-yl)-2-ethyl-3-hydroxy-3-methyltetrahydro-2H-pyran-4-yl acetate (37% yield, 99% ee) and (2R,3R,4R,6R)-6-(3-aminopyridin-4-yl)-2-ethyl-3-hydroxy-3-methyltetrahydro-2H-pyran-4-yl acetate (38% yield, 99% ee). LC/MS (m/z): 307.1 (MH⁺), Rₜ=0.44 min. ¹H NMR (400 MHz, CHLOROFORM-d) δ ppm 1.04 (t, 3H), 1.26 (s, 3H), 1.46-1.59 (m, 2H), 1.60-1.72 (m, 4H), 1.73-2.04 (m, 3H), 2.10 (s, 3H) overlapping with 2.11-2.25 m, 1H), 2.51 (broad s, 1H), 3.50 (m, 1H), 4.27 (s, 2H), 4.58 (1H, dd), 4.97 (1H, dd), 6.93 (d, 1H) 7.98 (d, 1H) 8.06 (s, 1H).

Method 6

### Synthesis of ((1-(cyclopent-1-en-1-yl)vinyl)oxy)triethylsilane

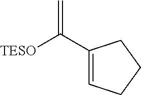

To a solution of LiHMDS (1.0 equiv.) in THF (0.5 M) cooled at −78° C. (internal thermometer) under N₂ was added a solution of 1-(cyclopent-1-en-1-yl)ethanone (1.0 equiv.) in THF (1.0 M) slowly over 50 min, keeping the internal temperature <−70° C. The resulting solution was stirred at −71° C. for 30 min before the dropwise addition of TES-Cl (1.10 equiv.) maintaining the internal temperature <−63° C. The cooling bath was then removed and the solution was allowed to warm to room temperature over 1.5 h. The reaction was poured into ice-cold saturated NaHCO₃ (400 mL) and Et₂O (1000 mL). The aqueous layer was separated and the organic layer was washed with NaHCO_{3(sat.)} (2×250 ml), brine then dried over Na₂SO₄, filtered and the volatiles were removed in vacuo to yield the desired product ((1-(cyclopent-1-en-1-yl)vinyl)oxy)triethylsilane as a colourless oil (99% yield). The oil was used without further purification. ¹H NMR (CHLOROFORM-d) δ: 6.01 (s, 1H), 4.27 (d, 2H), 2.44 (t, 3H), 1.94 (quin, 3H), 0.96-1.04 (m, 6H), 0.66-0.78 (m, 9H)

### Synthesis of cis (+/−)-3-nitro-4-((2R,7aR)-4-((triethylsilyl)oxy)-2,3,5,6,7,7a-hexahydrocyclopenta[b]pyran-2-yl)pyridine

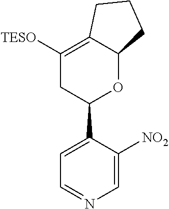

A solution of 3-nitroisonicotinaldehyde (1.0 equiv.), ((1-(cyclopent-1-en-1-yl)vinyl)oxy)triethylsilane (1.6 equiv.) and tris(6,6,7,7,8,8,8-heptafluoro-2,2-dimethyl-3,5-octanedionato) europium (0.05 equiv.) were dissolved in CHCl₃ (0.65 M) and stirred in a flame-dried round-bottom flask at 50° C. under an atmosphere of nitrogen for 1 hr. After this time the reaction mixture was cooled to room temperature and concentrated in vacuo to yield yellow oil. The oil was further purified by flash column chromatography by ISCO Combi-flash Rf system with a Redisep column eluting with 0-40% EtOAc/heptanes to afford the desired product cis (+/−)-3-nitro-4-((2R,7aR)-4-((triethylsilyl)oxy)-2,3,5,6,7,7a-hexahydrocyclopenta[b]pyran-2-yl)pyridine as a colourless oil (87% yield). LC/MS (m/z): 377.1 (MH⁺), Rₜ=0.89 min (65/95 method). ¹H NMR (400 MHz, CHLOROFORM-d) δ ppm 0.67 (q, 6H), 0.96-1.02 (m, 9H), 1.48-1.70 (m, 2H), 1.75-1.88 (m, 1H), 2.06-2.28 (m, 3H), 2.47-2.63 (m, 2H), 4.37-4.46 (m, 1H), 5.34 (dd, 1H), 7.85 (d, 1H), 8.88 (d, 1H), 9.24 (s, 1H).

### Synthesis of (+/−)-(2R,4aR,7aR)-4a-hydroxy-2-(3-nitropyridin-4-yl)hexahydrocyclopenta[b]pyran-4(4aH)-one

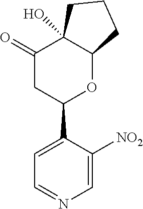 [SMILES: O=C1C[C@H](c2ccncc2[N+](=O)[O-])O[C@@H]2CCC[C@]12O]

To a solution of cis-(+/−)-3-nitro-4-((2R,7aR)-4-((triethylsilyl)oxy)-2,3,5,6,7,7a-hexahydrocyclopenta[b]pyran-2-yl)pyridine (1.0 equiv.) in DCM (0.2 M) cooled to 0° C. was added 3,3-dimethyldioxirane as a solution in acetone (0.1M solution, 1.00 equiv.) and allowed to stir for 20 min. To the reaction was added 5 mL of cyclohexene; the reaction mixture was stirred for 10 mins and the volatiles were removed in vacuo. The residue was taken up in THF (0.05 M) at room temperature and acidified with 5 mL of 1 M HCl (5.0 equiv.) the reaction stirred for 30 min. The solution was basified with 2 M NaOH to ˜pH=9. The product was extracted in EtOAc washed with brine, dried over MgSO₄, filtered and the volatiles were removed in vacuo. The oil was further purified by flash column chromatography by ISCO Combi-flash Rf system with a Redisep column eluting with 0-100% EtOAc/heptanes to afford as a single diastereoisomer the desired product (+/−)-(2R,4aR,7aR)-4a-hydroxy-2-(3-nitropyridin-4-yl)hexahydrocyclopenta[b]pyran-4(4aH)-one as a white solid (76% yield). LC/MS (m/z): 279.0 (MH⁺), Rₜ=0.58 min.

### Synthesis of (+/−)-(2R,4R,4aS,7aR)-2-(3-nitropyridin-4-yl)octahydrocyclopenta[b]pyran-4,4a-diol

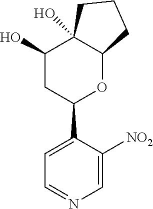 [SMILES: O=[N+]([O-])c1cnccc1[C@H]1C[C@@H](O)[C@@]2(O)CCC[C@H]2O1]

To a solution of (2R,4aR,7aR)-4a-hydroxy-2-(3-nitropyridin-4-yl)hexahydrocyclopenta[b]pyran-4(4aH)-one (1.0 equiv.) in EtOH (0.1 M) at 0° C. was added sodium borohydride (1.0 equiv.). The reaction mixture was then stirred at 0° C. for 45 min. The reaction mixture was then quenched by the addition of water and stirred for 5 min before being concentrated in vacuo, the resulting residue was then partitioned between water and EtOAc. The aqueous layer was then separated and extracted with EtOAc (×2) the combined organics were then washed with brine, dried over Na₂SO₄, filtered, and the volatiles were removed in vacuo to yield the desired product as predominately a single diastereoisomer (+/−)-(2R,4R,4aS,7aR)-2-(3-nitropyridin-4-yl)octahydrocyclopenta[b]pyran-4,4a-diol (81% yield) as a white solid. LC/MS (m/z): 281.1 (MH⁺), Rₜ=0.47 min. ¹H NMR (DMSO-d₆) δ ppm: 9.10 (s, 1H), 8.84 (d, 1H), 7.70 (d, 1H), 4.89-4.93 (m, 2H), 4.56 (s, 1H), 3.93 (ddd, 1H), 3.58 (d, 1H), 2.14 (ddd, 1H), 1.97-2.07 (m, 1H), 1.86-1.95 (m, 1H), 1.68-1.78 (m, 2H), 1.41-1.59 (m, 3H). The resulting solid was used in the subsequent transformation without further purification.

### Synthesis of (+/−)-(2R,4R,4aR,7aR)-4a-hydroxy-2-(3-nitropyridin-4-yl)octahydrocyclopenta[b]pyran-4-yl acetate

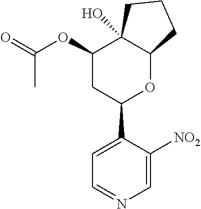 [SMILES: CC(=O)O[C@@H]1C[C@H](c2ccncc2[N+](=O)[O-])O[C@@H]2CCC[C@]12O]

To a solution of (+/−)-(2R,4R,4aS,7aR)-2-(3-nitropyridin-4-yl)octahydrocyclopenta[b]pyran-4,4a-diol (1.0 equiv.) in pyridine (0.20 M) at room temperature was added acetic anhydride (5.0 equiv.). The reaction mixture was stirred overnight at room temperature after which time the mixture was concentrated in vacuo. The reaction was then pardoned between water and EtOAc. The organics were washed with CuSO₄ (10% aq.), brine then dried over Na₂SO₄, filtered, and volatiles were removed in vacuo. The residue was further purified by flash column chromatography by ISCO Combi-flash Rf system with a Redisep column eluting with 0-100% EtOAc/heptanes to afford the desired product (+/−)-(2R,4R,4aR,7aR)-4a-hydroxy-2-(3-nitropyridin-4-yl)octahydrocyclopenta[b]pyran-4-yl acetate as a white solid (76% yield). LC/MS (m/z): 323.0 (MH⁺) Rₜ=0.67 min. ¹H NMR (CHLOROFORM-d) δ: 9.17 (s, 1H), 8.82 (d, 1H), 7.72 (d, 1H), 5.34 (dd, 1H), 5.15 (dd, 1H), 3.84 (d, 1H), 3.12 (br. s., 1H), 2.44 (ddd, 1H), 2.15-2.28 (m, 1H), 2.02-2.14 (m, 4H), 1.86-1.97 (m, 2H), 1.75-1.85 (m, 1H), 1.57-1.70 (m, 1H).

### Synthesis of (2S,4S,4aS,7aS)-2-(3-aminopyridin-4-yl)-4a-hydroxyoctahydrocyclopenta[b]pyran-4-yl acetate and (2R,4R,4aR,7aR)-2-(3-aminopyridin-4-yl)-4a-hydroxyoctahydrocyclopenta[b]pyran-4-yl acetate

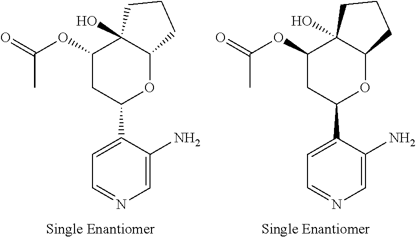

A solution of (+/−)-(2R,4R,4aR,7aR)-4a-hydroxy-2-(3-nitropyridin-4-yl)octahydrocyclopenta[b]pyran-4-yl acetate (1.0 equiv.) in EtOH (0.1 M) was degassed with argon for 20 min. At room temperature under an Argon atmosphere, 10% Pd/C (10 mol %) was added and the resulting mixture was evacuated and backfilled with hydrogen gas (three times) and the mixture was then stirred at room temperature under atmospheric partial pressure of hydrogen gas (balloon) for 2.5 h. The reaction was filtered, and the volatiles were removed in vacuo to yield a white solid. Purification was completed via chiral SFC (CO₂/EtOH+0.1% DEA=50/50, 15 mL/min, AD column) to yield in order of elution (2S,4S,4aS,7aS)-2-(3-aminopyridin-4-yl)-4a-hydroxyoctahydrocyclopenta[b]pyran-4-yl acetate (36% yield, 99% ee) and (2R,4R,4aR,7aR)-2-(3-aminopyridin-4-yl)-4a-hydroxyoctahydrocyclopenta[b]pyran-4-yl acetate (38% yield, 99% ee).

LC/MS (m/z): 293.0 (MH⁺), Rₜ=0.39 min. ¹H NMR (CHLOROFORM-d) δ: 8.05 (s, 1H), 7.98 (d, 1H), 6.94 (d, 1H), 5.27 (dd, 1H), 4.52 (dd, 1H), 4.17 (br. s., 2H), 3.85 (d, 1H), 2.04-2.30 (m, 5H), 1.75-1.98 (m, 2H), 1.62-1.73 (m, 1H).

### Synthesis of (+/−)-(2R,4aS,7aR)-2-(3-nitropyridin-4-yl)hexahydrocyclopenta[b]pyran-4(4aH)-one

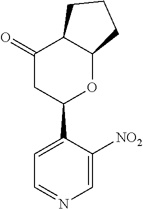 [SMILES: O=C1C[C@H](c2ccncc2[N+](=O)[O-])O[C@@H]2CCC[C@H]12]

A solution of cis (+/−)-3-nitro-4-((2R,7aR)-4-((triethylsilyl)oxy)-2,3,5,6,7,7a-hexahydrocyclopenta[b]pyran-2-yl)pyridine (1.0 equiv.) in THF/1M HCl (4:1, 0.1M) was stirred at rt for 2 hours. The solution was neutralized with 1M NaOH and the THF was removed under vacuo. The mixture was diluted with ethyl acetate and the organic phase was washed with sat. sodium bicarbonate. The organic solution was dried with sodium sulfate, filtered and concentrated to give (+/−)-(2R,4aS,7aR)-2-(3-nitropyridin-4-yl)hexahydrocyclopenta[b]pyran-4(4aH)-one in 93% yield.

LC/MS (m/z): 263.1 (MH⁺), Rₜ=0.73 min.

### Synthesis of (+/−)-(2R,4R,4aR,7aR)—N-benzyl-2-(3-nitropyridin-4-yl)octahydrocyclopenta[b]pyran-4-amine

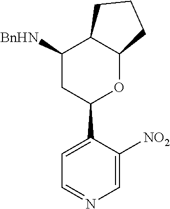

To a solution of (+/−)-(2R,4aS,7aR)-2-(3-nitropyridin-4-yl)hexahydrocyclopenta[b]pyran-4(4aH)-one (1.0 equiv.) in MeOH was added benzyl amine (3.0 equiv.) and the reaction was stirred at rt for 2 h. Cooled to −78° C. and added 2M LiBH₄ (THF solution, 1.1 equiv.) dropwise. The mixture was allowed to warm to rt and stirred overnight. Diluted with ethyl acetate and washed with sat. sodium bicarbonate. Washed with brine, dried over sodium sulfate, filtered and concentrated to give (+/−)-(2R,4R,4aR,7aR)—N-benzyl-2-(3-nitropyridin-4-yl)octahydrocyclopenta[b]pyran-4-amine in 92% yield. LC/MS (m/z): 354.1 (MH⁺), Rₜ=0.62 min.

### Synthesis of tert-butyl ((2S,4S,4aS,7aS)-2-(3-aminopyridin-4-aminopyridin-4-yl)octahydrocyclopenta[b]pyran-4-yl)carbamate

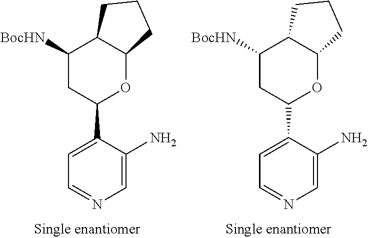

To a degassed solution of (+/−)-(2R,4R,4aR,7aR)—N-benzyl-2-(3-nitropyridin-4-yl)octahydrocyclopenta[b]pyran-4-amine (1.0 equiv.) in MeOH (0.1M) was added Pd(OH)₂ (0.2 equiv.) and the reaction was stirred under a hydrogen balloon for 17 h. The solution was purged with nitrogen and Boc₂O (2.0 equiv.) was added and stirred at rt for 2 h. The solution was filtered through Celite and washed with ethyl acetate. Upon concentration of the solvent, (+/−)-tert-butyl ((2S,4S,4aS,7aS)-2-(3-aminopyridin-4-yl)octahydrocyclopenta[b]pyran-4-yl)carbamate was obtained. Purification was completed via chiral HPLC (heptane:EtOH=80/20, 15 mL/min, IC column) to yield in order of elution tert-butyl ((2S,4S,4aS,7aS)-2-(3-aminopyridin-4-yl)octahydrocyclopenta[b]pyran-4-yl)carbamate (18% yield, >99% ee) and tert-butyl ((2R,4R,4aR,7aR)-2-(3-aminopyridin-4-yl)octahydrocyclopenta[b]pyran-4-yl)carbamate (16% yield, >99% ee). LC/MS (m/z): 334.2 (MI-1′), Rₜ=0.66 min.

### Synthesis of (E)-4-cyclopropylbut-3-en-2-one

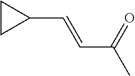

To a solution of cyclopropanecarbaldehyde (1.0 equiv.) and acetone (19.63 equiv.) in DMSO (0.174 M) at RT was added (S)-pyrrolidine-2-carboxylic acid (25 mol %). The resulting mixture was stirred at RT for 16 h. The reaction mixture was then quenched by addition of NH₄Cl. The aqueous phase was then separated and extracted with EtOAc. The combined organics were then washed successively with aq.sat. NaHCO₃ (×²) followed by brine. The organic layer was then dried over Na₂SO₄, filtered and concentrated in vacuo to yield a colourless oil. The oil was further purified by flash column chromatography by ISCO Combi-flash Rf system with a Redisep column eluting with 0-100% EtOAc/heptanes to afford the desired product (E)-4-cyclopropylbut-3-en-2-one as a solution in 1:1 Et₂O:heptanes which was used in the subsequent transformation without further manipulation. LC/MS (m/z): 110.9 (MH⁺), Rₜ=0.57 min. ¹H NMR (400 MHz, CHLOROFORM-d) δ ppm: 6.28 (dd, 1H), 6.18 (d, 1H), 2.20 (s, 3H), 1.51-1.65 (m, 1H), 0.91-1.07 (m, 2H), 0.57-0.74 (m, 2H).

### Synthesis of (E)-((4-cyclopropylbuta-1,3-dien-2-yl)oxy)triethylsilane

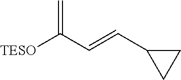

To a solution of (E)-4-cyclopropylbut-3-en-2-one (1.0 equiv.) and triethylamine (1.4 equiv.) in heptane:Et₂O (10:1, 0.08 M) cooled to 0° C. was added triethylsilyl trifluoromethanesulfonate (1.0 equiv.) dropwise over five minutes. The resulting mixture was stirred at 0° C. for 2 h. The reaction mixture was then quenched with NaHCO₃, the aqueous layer was separated and extracted with Et₂O. The combined organics were then dried over MgSO₄, filtered and concentrated in vacuo to yield the desired product (E)-triethyl((3-methylhexa-1,3-dien-2-yl)oxy)silane as a colourless oil (yield=84%) which was used in the Hetero-Diels Alder reaction without further purification.

### Synthesis of cis (+/−)-4-((2R,6R)-6-cyclopropyl-4-((triethylsilyl)oxy)-3,6-dihydro-2H-pyran-2-yl)-3-nitropyridine

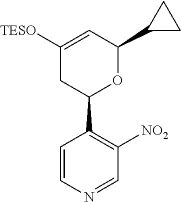

A solution of 3-nitroisonicotinaldehyde (1.5 equiv.), (E)-triethyl((3-methylhexa-1,3-dien-2-yl)oxy)silane (1.0 equiv.), and tris(6,6,7,7,8,8,8-heptafluoro-2,2-dimethyl-3,5-octanedionato) europium (0.05 equiv.) were dissolved in CHCl₃ (0.16 M) and stirred in a flame-dried round-bottom flask at 60° C. under an atmosphere of nitrogen for 1 hr. After this time the reaction mixture was cooled to room temperature and concentrated in vacuo to yield yellow oil. The oil was further purified by flash column chromatography by ISCO Combi-flash Rf system with a Redisep column eluting with 0-30% Et₂O/heptanes with 1% Et₃N to afford the desired product cis (+/−)-4-((2R,6R)-6-cyclopropyl-4-((triethylsilyl)oxy)-3,6-dihydro-2H-pyran-2-yl)-3-nitropyridine as a colourless oil (63% yield over three steps). LC/MS (m/z): 377.2 (MH⁺), Rₜ=1.36 min. ¹H NMR (400 MHz, CHLOROFORM-d) δ ppm 0.28-0.39 (m, 1H), 0.41-0.51 (m, 1H), 0.51-0.67 (m, 2H), 0.74 (q, 6H), 0.88 (t, 1H) 1.04 (t, 9H) 1.17-1.35 (m, 1H) 2.32-2.45 (m, 1H) 2.54-2.66 (m, 1H) 3.67-3.76 (m, 1H) 5.03 (s, 1H) 5.35-5.46 (m, 1H) 8.09 (d, 1H) 9.46-9.70 (m, 1H) 9.82-10.09 (m, 1H)

### Synthesis of (+/−)-(2R,3R,6R)-2-cyclopropyl-3-hydroxy-6-(3-nitropyridin-4-yl)dihydro-2H-pyran-4(3H)-one

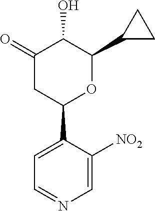 [SMILES: O=C1C[C@H](c2ccncc2[N+](=O)[O-])O[C@H](C2CC2)[C@H]1O]

To a solution of cis-(+/−)-4-((2R,6R)-6-cyclopropyl-4-((triethylsilyl)oxy)-3,6-dihydro-2H-pyran-2-yl)-3-nitropyridine (1.0 equiv.) in EtOAc:water 1:1(0.08 M) was added acetone (10.0 equiv.), NaHCO₃ (5.00 equiv.) at RT. To the resulting solution was added a solution of oxone (1.00 equv.) in water (0.16 M) dropwise by addition funnel taking care to keep the internal reaction temperature below 20° C. The reaction mixture was stirred at RT for 3 h before being quenched with cyclohexene (5 ml) and diluted with EtOAc and brine. The organic layer was then separated, dried over Na₂SO₄, filtered and the volatiles were removed in vacuo. The residue was taken up in THF (0.05 M) at room temperature and acidified with 1 M HCl (1.5 equiv.) the reaction mixture was then stirred for 1 h at RT. The reaction mixture was then quenched with NaHCO₃ (sat.). The aqueous layer was separated and extracted with EtOAc. The combined organics were then dried over Na₂SO₄, filtered and concentrated in vacuo to yield a colourless oil. The oil was further purified by flash column chromatography by ISCO Combi-flash Rf system with a Redisep column eluting with 0-23% EtOAc/heptanes to afford as a single diastereoisomer the desired product (+/−)-(2R,3R,6R)-2-cyclopropyl-3-hydroxy-6-(3-nitropyridin-4-yl)dihydro-2H-pyran-4(3H)-one as a colourless oil (35% yield). LC/MS (m/z): 279.0 (MH⁺), Rₜ=0.59 min (0/95 method). ¹H NMR (400 MHz, CHLOROFORM-d) δ ppm 0.46-0.55 (m, 1H), 0.55-0.64 (m, 2H), 0.65-0.78 (m, 1H), 1.23-1.37 (m, 1H), 2.56-2.68 (m, 1H), 3.08-3.20 (m, 2H), 3.64 (d, 1H), 4.18 (d, 1H), 5.28 (dd, 1H), 7.81 (d, 1H), 8.90 (d, 1H), 9.23 (s, 1H).

### Synthesis of (+/−)-(2R,3S,4R,6R)-2-cyclopropyl-6-(3-nitropyridin-4-yl)tetrahydro-2H-pyran-3,4-diol

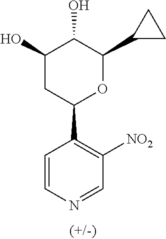 [SMILES: O=[N+]([O-])c1cnccc1[C@H]1C[C@@H](O)[C@H](O)[C@@H](C2CC2)O1]

To a solution of (+/−)-(2R,3R,6R)-2-cyclopropyl-3-hydroxy-6-(3-nitropyridin-4-yl)dihydro-2H-pyran-4(3H)-one (1.0 equiv.) in EtOH (0.21 M) at 0° C. was added sodium borohydride (1.1 equiv.). The reaction mixture was allowed to stir for 30 min warming to room temperature. The reaction mixture was then concentrated and partitioned between water and EtOAc. The aqueous layer was then separated and extracted with EtOAc (×2) the combined organics were then washed with brine, dried over Na₂SO₄, filtered, and the volatiles were removed in vacuo to yield a mixture of C4 epimers (9:1 as determined by analytical UPLC). The oil was further purified by flash column chromatography by ISCO Combi-flash Rf system with a Redisep column eluting with 0-60% EtOAc/DCM to afford as a single diastereoisomer the desired product (+/−)-(2R,3S,4R,6R)-2-cyclopropyl-6-(3-nitropyridin-4-yl)tetrahydro-2H-pyran-3,4-diol as a colourless oil (46% yield). LC/MS (m/z): 281.1 (MH⁺), Rₜ=0.50 min (0-95 method). ¹H NMR (400 MHz, CHLOROFORM-d) δ ppm 0.40 (dq, J=5.72, 5.53 Hz, 1H), 0.45-0.56 (m, 2H), 0.57-0.69 (m, 1H), 1.02-1.15 (m, 1H), 1.55 (q, 1H), 2.39-2.53 (m, 1H), 2.87 (dd, 1H), 3.41-3.59 (m, 2H), 3.83-3.98 (m, 2H), 5.07 (d, 1H), 7.75 (d, 1H), 8.82 (d, 1H), 9.16 (s, 1H).

### Synthesis of (+/−)-(2R,3S,4R,6R)-2-cyclopropyl-6-(3-nitropyridin-4-yl)tetrahydro-2H-pyran-3,4-diyl diacetate

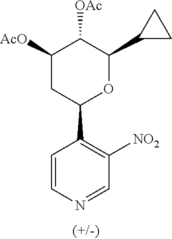 [SMILES: CC(=O)O[C@@H]1[C@@H](C2CC2)O[C@@H](c2ccncc2[N+](=O)[O-])C[C@H]1OC(C)=O]

To a solution of (+/−)-(2R,3S,4R,6R)-2-cyclopropyl-6-(3-nitropyridin-4-yl)tetrahydro-2H-pyran-3,4-diol (1.0 equiv.) in pyridine (0.195 M) at room temperature was added acetic anhydride (6.0 equiv.). The reaction mixture was stirred for 7 hr at room temperature. The reaction was quenched with water and the product was extracted in EtOAc and washed with brine. The organics were dried over MgSO₄, filtered, and volatiles were removed in vacuo to yield a colourless oil (unpurified mass recovery=99%). The oil was used in without further purification.

### Synthesis of (2S,3S,4S,6S)-6-(3-aminopyridin-4-yl)-2-ethyl-3-hydroxy-3-methyltetrahydro-2H-pyran-4-yl acetate and (2R,3R,4R,6R)-6-(3-aminopyridin-4-yl)-2-ethyl-3-hydroxy-3-methyltetrahydro-2H-pyran-4-yl acetate

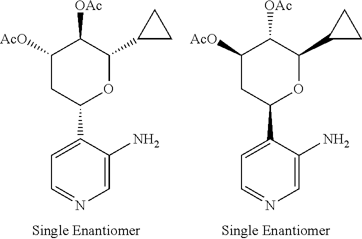

To a solution of (+/−)-(2R,3S,4R,6R)-2-cyclopropyl-6-(3-nitropyridin-4-yl)tetrahydro-2H-pyran-3,4-diyl diacetate (1.0 equiv.) in AcOH (0.116 M) at RT was added Iron powder (10.0 equiv.). The reaction mixture was stirred at RT for 1 h. After this time the reaction mixture was concentrated to dryness diluted with EtOAc and NaHCO₃. The organic layer was then separated and washed with NaHCO₃, brine, dried over Na₂SO₄, filtered, and volatiles were removed in vacuo to yield a colourless oil. The oil was further purified by flash column chromatography by ISCO Combi-flash Rf system with a Redisep column eluting with 0-100% EtOAc/heptanes to afford a colourless oil. Further chiral separation and purification was completed via chiral HPLC (heptane/EtOH=85/15, 1 mL/min, OJ-H column) to yield in order of elution ((2S,3R,4S,6S)-6-(3-aminopyridin-4-yl)-2-cyclopropyltetrahydro-2H-pyran-3,4-diyl diacetate (43% yield, 99% ee) and ((2R,3S,4R,6R)-6-(3-aminopyridin-4-yl)-2-cyclopropyltetrahydro-2H-pyran-3,4-diyl diacetate (43% yield, 99% ee). LC/MS (m/z): 335.1 (MH⁺), Rₜ=0.53 min. ¹H NMR (400 MHz, CHLOROFORM-d) δ ppm 0.27-0.42 (m, 2H), 0.50-0.63 (m, 2H), 0.98 (td, 1H), 2.01-2.22 (m, 7H), 2.31-2.39 (m, 1H), 2.80 (t, 1H), 4.24 (br. s., 2H), 4.48 (dd, 1H), 5.05-5.17 (m, 2H), 6.93 (d, 1H), 7.99 (d, 1H), 8.07 (s, 1H).

### Synthesis of (+/−)-(2R,4R,4aS,7aR)-4-(benzylamino)-2-(3-nitropyridin-4-yl)octahydrocyclopenta[b]pyran-4a-ol

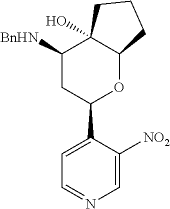

To a solution of (+/−)-(2R,4aR,7aR)-4a-hydroxy-2-(3-nitropyridin-4-yl)hexahydrocyclopenta[b]pyran-4(4aH)-one in MeOH (0.2 M) at RT was added benzyl amine (3.0 equiv.). The reaction mixture was then stirred at RT for 2 h before being cooled to −78° C. followed by the dropwise addition of LiBH₄ (1.10 equiv.). The reaction mixture was then allowed to warm to RT overnight. The reaction mixture was then quenched by the addition of NaHCO₃ and diluted with EtOAc. The organic layer was then separated and washed with NaHCO₃ (×2), brine, dried over Na₂SO₄, filtered, and the volatiles were removed in vacuo to yield an off white solid. The solid was further purified by flash column chromatography by ISCO Combi-flash Rf system with a Redisep column eluting with 0-10% MeOH/DCM to afford the desired product (+/−)-(2R,4R,4aS,7aR)-4-(benzylamino)-2-(3-nitropyridin-4-yl)octahydrocyclopenta[b]pyran-4a-ol as a white solid (58% yield). LC/MS (m/z): 370.1 (MH⁺), Rₜ=0.56 min. ¹H NMR (CHLOROFORM-d₆) δ ppm: 9.18 (s, 1H), 8.80 (d, 2H), 7.72 (d, 2H), 7.32-7.36 (m, 4H), 7.24-7.30 (m, 1H), 5.07 (dd, 1H), 3.96 (d, 1H), 3.75 (d, 1H), 3.72 (d, 1H), 3.14 (dd, 1H), 2.54 (ddd, 2H), 2.09-2.22 (m, 1H), 1.86-2.06 (m, 3H), 1.71-1.82 (m, 1H), 1.50-1.63 (m, 1H), 1.19-1.34 (m, 1H).

### Synthesis of tert-butyl ((2S,4S,4aR,7aS)-2-(3-aminopyridin-4-yl)-4a-hydroxyoctahydrocyclopenta[b]pyran-4-yl)carbamate and tert-butyl ((2R,4R,4aS,7aR)-2-(3-aminopyridin-4-yl)-4a-hydroxyoctahydrocyclopenta[b]pyran-4-yl)carbamate

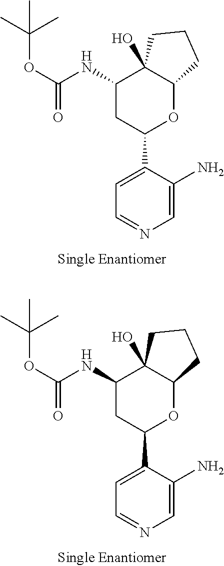 [SMILES: CC(C)(C)OC(=O)N[C@H]1C[C@@H](c2ccncc2N)O[C@H]2CCC[C@@]12O ; CC(C)(C)OC(=O)N[C@@H]1C[C@H](c2ccncc2N)O[C@@H]2CCC[C@]12O]

A suspension of (+/−)-(2R,4R,4aS,7aR)-4-(benzylamino)-2-(3-nitropyridin-4-yl)octahydrocyclopenta[b]pyran-4a-ol (1.0 equiv.) in MeOH (0.2 M) was degassed with argon for 20 min. At room temperature under an Argon atmosphere, 10% Pearlman's catalyst (Pd hydroxide) (10 mol %) was added and the resulting mixture was evacuated and backfilled with hydrogen gas (three times) and the mixture was then stirred at room temperature under atmospheric partial pressure of hydrogen gas (balloon) for 17 h. The hydrogen gas was then removed by evacuation and the reaction vessel back filled with argon. To the reaction mixture was then added Boc anhydride (2.00 equv.) at RT and the reaction mixture was stirred for 2 h. The reaction mixture was then filtered through celite and the volatiles were removed in vacuo to yield a crude residue. The residue was further purified by flash column chromatography by ISCO Combi-flash Rf system with a Redisep column eluting with 0-10% MeOH/DCM to afford a colourless oil. Purification was completed via chiral HPLC (EtOH/heptane=40/60, 20 mL/min, AD column) to yield in order of elution tert-butyl ((2S,4S,4aR,7aS)-2-(3-aminopyridin-4-yl)-4a-hydroxyoctahydrocyclopenta[b]pyran-4-yl)carbamate (32% yield, 99% ee) and tert-butyl ((2R,4R,4aS,7aR)-2-(3-aminopyridin-4-yl)-4a-hydroxyoctahydrocyclopenta[b]pyran-4-yl)carbamate (33% yield, 99% ee).

LC/MS (m/z): 350.2 (MH⁺), Rₜ=0.50 min. ¹H NMR (CHLOROFORM-d) δ: 8.04 (s, 1H), 7.97 (d, 1H), 6.91 (d, 1H), 4.71 (br. s., 1H), 4.50 (dd, 1H), 4.39 (br. s., 1H), 4.19 (s, 2H), 4.10 (dt, 1H), 3.78 (d, 1H), 2.18-2.31 (m, 1H), 2.06 (ddd, 1H), 1.71-1.99 (m, 5H), 1.57-1.68 (m, 1H), 1.46 (s, 9H).

### Synthesis of (+/−)-(2R,3S,4R,6R)-4-(benzylamino)-2,3-dimethyl-6-(3-nitropyridin-4-yl)tetrahydro-2H-pyran-3-ol

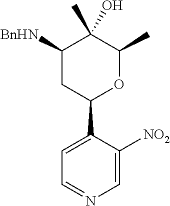

To a solution of (+/−)-(2R,3R,6R)-3-hydroxy-2,3-dimethyl-6-(3-nitropyridin-4-yl)dihydro-2H-pyran-4(3H)-one in MeOH (0.2 M) at RT was added benzyl amine (3.0 equiv.). The reaction mixture was then stirred at RT for 2 h before being cooled to −78° C. followed by the dropwise addition of LiBH₄ (1.10 equiv.). The reaction mixture was then allowed to warm to RT overnight. The reaction mixture was then quenched by the addition of NaHCO₃ and diluted with EtOAc. The organic layer was then separated and washed with NaHCO₃ (×2), brine, dried over Na₂SO₄, filtered, and the volatiles were removed in vacuo to yield an off white solid. The solid was further purified by flash column chromatography by ISCO Combi-flash Rf system with a Redisep column eluting with 0-10% MeOH/DCM to afford the desired product (+/−)-(2R,3S,4R,6R)-4-(benzylamino)-2,3-dimethyl-6-(3-nitropyridin-4-yl)tetrahydro-2H-pyran-3-ol as a white solid (99% yield). LC/MS (m/z): 358.1 (MH⁺) Rₜ=0.56 min. ¹H NMR (CHLOROFORM-d₆) δ ppm: 9.16 (s, 1H), 8.80 (d, J=5.1 Hz, 1H), 7.78 (d, J=5.1 Hz, 1H), 7.32-7.35 (m, 3H), 7.23-7.30 (m, 2H), 5.14 (dd, J=11.0, 2.3 Hz, 1H), 3.72-3.98 (m, 2H), 3.49 (q, J=6.3 Hz, 1H), 2.78 (dd, J=11.9, 4.1 Hz, 1H), 2.53 (ddd, J=12.8, 4.2, 2.5 Hz, 1H), 1.25 (d, J=6.3 Hz, 3H), 1.16 (s, 3H).

### Synthesis of tert-butyl ((2S,3R,4S,6S)-6-(3-aminopyridin-4-yl)-3-hydroxy-2,3-dimethyltetrahydro-2H-pyran-4-yl)carbamate and tert-butyl ((2R,3S,4R,6R)-6-(3-aminopyridin-4-yl)-3-hydroxy-2,3-dimethyltetrahydro-2H-pyran-4-yl)carbamate

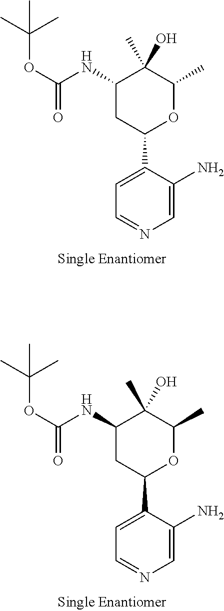

A solution of (+/−)-(2R,3S,4R,6R)-4-(benzylamino)-2,3-dimethyl-6-(3-nitropyridin-4-yl)tetrahydro-2H-pyran-3-ol (1.0 equiv.) in MeOH:EtOAc (4:1, 0.2 M) was degassed with argon for 20 min. At room temperature under an Argon atmosphere, 10% Pearlman's catalyst (Pd hydroxide) (10 mol %) was added and the resulting mixture was evacuated and backfilled with hydrogen gas (three times) and the mixture was then stirred at room temperature under atmospheric partial pressure of hydrogen gas (balloon) for 17 h. The hydrogen gas was then removed by evacuation and the reaction vessel back filled with argon. To the reaction mixture was then added Boc anhydride (2.00 equv.) at RT and the reaction mixture was stirred for 2 h. The reaction mixture was then filtered through celite and the volatiles were removed in vacuo to yield a crude residue. The residue was further purified by flash column chromatography by ISCO Combi-flash Rf system with a Redisep column eluting with 0-10% MeOH/DCM to afford a colourless oil. Purification was completed via chiral HPLC (EtOH/heptane=40/60, 20 mL/min, AD column) to yield in order of elution tert-butyl ((2S,4S,4aR,7aS)-2-(3-aminopyridin-4-yl)-4a-hydroxyoctahydrocyclopenta[b]pyran-4-yl)carbamate (12% yield, 99% ee) and tert-butyl ((2R,4R,4aS,7aR)-2-(3-aminopyridin-4-yl)-4a-hydroxyoctahydrocyclopenta[b]pyran-4-yl)carbamate (12% yield, 99% ee).

LC/MS (m/z): 338.1 (MH⁺) Rₜ=0.48 min. ¹H NMR (CHLOROFORM-d) δ: 1.14 (s, 3H) 1.27 (d, 3H) 1.44 (s, 9H) 1.80-2.02 (m, 2H) 3.53 (q, 1H) 3.82 (ddd, 1H) 4.28 (br. s., 2H) 4.36 (br. s., 1H) 4.56 (dd, 1H) 4.96 (d, 1H) 6.89 (d, 1H) 7.94 (d, 1H) 8.02 (s, 1H).

### Synthesis of (+/−)-(2R,3S,4R,6R)-4-(benzylamino)-3-ethyl-2-methyl-6-(3-nitropyridin-4-yl)tetrahydro-2H-pyran-3-ol

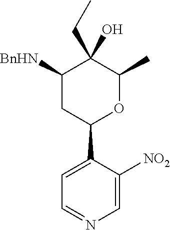

To a solution of (+/−)-(2R,3R,6R)-3-ethyl-3-hydroxy-2-methyl-6-(3-nitropyridin-4-yl)dihydro-2H-pyran-4(3H)-one in MeOH (1.0 M) at RT was added 4A molecular sieves (50 mg) followed by benzyl amine (3.0 equiv.). The reaction mixture was then stirred at RT for 20 h before being cooled to −78° C. followed by the dropwise addition of LiBH₄ (1.10 equiv.). The reaction mixture was then stirred at −78° C. for 3 h. The reaction mixture was then quenched by the addition of NaHCO₃ and diluted with EtOAc. The organic layer was then separated and washed with NaHCO₃ (×2), brine, dried over MgSO₄, filtered, and the volatiles were removed in vacuo to yield an off white solid. The solid was further purified by flash column chromatography by ISCO Combi-flash Rf system with a Redisep column eluting with 0-40% EtOAc/heptanes to afford in order of elution the desired product (+/−)-(2R,3S,4R,6R)-4-(benzylamino)-2,3-dimethyl-6-(3-nitropyridin-4-yl)tetrahydro-2H-pyran-3-ol as a white solid (24% yield). LC/MS (m/z): 372.1 (MH⁺) Rₜ=0.60 min. ¹H NMR (CHLOROFORM-d₆) δ ppm: 9.17 (s, 1H), 8.81 (d, 1H), 7.78 (d, 1H), 7.30-7.41 (m, 5H), 5.15 (dd, 1H), 3.95 (d, 1H), 3.74 (d, 1H), 3.44-3.56 (m, 1H), 2.80 (dd, 2H), 2.45-2.54 (dt, 1H), 1.77-1.90 (m, 2H), 1.39-1.67 (m, 1H), 1.27 (q, 2H) overlapping with 1.27 (d, 3H), 1.06 (t, 3H). Followed by the other reductive amination diastereoisomer, (2R,3S,4S,6R)-4-(benzylamino)-3-ethyl-2-methyl-6-(3-nitropyridin-4-yl)tetrahydro-2H-pyran-3-ol (22% yield).

### Synthesis of tert-butyl ((2R,3S,4R,6R)-6-(3-aminopyridin-4-yl)-3-ethyl-3-hydroxy-2-methyltetrahydro-2H-pyran-4-yl)carbamate and tert-butyl ((2S,3R,4S,6S)-6-(3-aminopyridin-4-yl)-3-ethyl-3-hydroxy-2-methyltetrahydro-2H-pyran-4-yl)carbamate

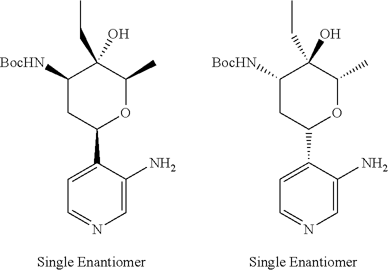

A solution of (+/−)-(2R,3S,4R,6R)-4-(benzylamino)-3-ethyl-2-methyl-6-(3-nitropyridin-4-yl)tetrahydro-2H-pyran-3-ol (1.0 equiv.) in MeOH:EtOAc (4:1, 0.2 M) was degassed with argon for 20 min. At room temperature under an Argon atmosphere, 10% Pearlman's catalyst (Pd hydroxide) (10 mol %) was added and the resulting mixture was evacuated and backfilled with hydrogen gas (three times) and the mixture was then stirred at room temperature under atmospheric partial pressure of hydrogen gas (balloon) for 19 h. The hydrogen gas was then removed by evacuation and the reaction vessel back filled with argon. To the reaction mixture was then added Boc anhydride (2.00 equv.) at RT and the reaction mixture was stirred for 2 h. The reaction mixture was then filtered through celite and the volatiles were removed in vacuo to yield a crude residue. The residue was further purified by flash column chromatography by ISCO Combi-flash Rf system with a Redisep column eluting with 0-10% MeOH/DCM to afford a colourless oil. Purification was completed via chiral HPLC (EtOH/heptane=25/75, 20 mL/min, AD column) to yield in order of elution tert-butyl ((2S,4S,4aR,7aS)-2-(3-aminopyridin-4-yl)-4a-hydroxyoctahydrocyclopenta[b]pyran-4-yl)carbamate (20% yield, 99% ee) and tert-butyl ((2R,4R,4aS,7aR)-2-(3-aminopyridin-4-yl)-4a-hydroxyoctahydrocyclopenta[b]pyran-4-yl)carbamate (18% yield, 99% ee).

LC/MS (m/z): 352.2 (MH⁺), Rₜ=0.59 min. ¹H NMR (CHLOROFORM-d) δ: 8.04 (s, 1H), 7.97 (d, 1H), 6.86-6.95 (m, 1H), 4.63-4.74 (m, 1H), 4.57 (dd, 1H), 4.49 (br. s., 1H), 4.24 (br. s., 2H), 3.77-3.90 (m, 1H), 3.51-3.56 (m, 1H), 1.88-1.99 (m, 1H), 1.67-1.79 (m, 2H), 1.56-1.65 (m, 1H), 1.46 (s, 9H), 1.29 (d, 3H), 1.06 (t, 3H).

### Synthesis of (2R,4R,4aS,8aR)-4-(benzylamino)-2-(3-nitropyridin-4-yl)octahydro-2H-chromen-4a-ol

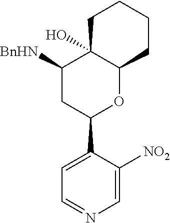

To a solution of (+/−)-(2R,4aR,8aR)-4a-hydroxy-2-(3-nitropyridin-4-yl)hexahydro-2H-chromen-4(3H)-one in MeOH (0.2 M) at RT was added benzyl amine (3.0 equiv.). The reaction mixture was then stirred at RT for 3 h before being cooled to −78° C. followed by the dropwise addition of LiBH₄(1.10 equiv.). The reaction mixture was then allowed to warm to RT overnight. The reaction mixture was then quenched by the addition of NaHCO₃ and diluted with EtOAc. The organic layer was then separated and washed with NaHCO₃ (×2), brine, dried over Na₂SO₄, filtered, and the volatiles were removed in vacuo to yield an off white solid. The solid was further purified by flash column chromatography by ISCO Combi-flash Rf system with a Redisep column eluting with 0-100% EtOAc/heptanes to afford the desired product (+/−)-(2R,4R,4aS,8aR)-4-(benzylamino)-2-(3-nitropyridin-4-yl)octahydro-2H-chromen-4a-ol as a white solid (57% yield). LC/MS (m/z): 384.1 (MH⁺) Rₜ=0.60 min. ¹H NMR (CHLOROFORM-d₆) δ ppm: 1.27-1.38 (m, 1H) 1.42-1.81 (m, 8H) 1.89 (dd, 1H) 2.54 (ddd, 1H) 2.73 (s, 1H) 2.80 (dd, 1H) 3.42 (s, 1H) 3.72 (d, 1H) 3.94 (d, 1H) 5.14 (dd, 1H) 7.24-7.30 (m, 1H) 7.30-7.37 (m, 4H) 7.81 (d, 1H) 8.82 (d, 1H) 9.18 (s, 1H).

### Synthesis of tert-butyl ((2R,4R,4aS,8aR)-2-(3-aminopyridin-4-yl)-4a-hydroxyoctahydro-2H-chromen-4-yl)carbamate and tert-butyl ((2S,4S,4aR,8aS)-2-(3-aminopyridin-4-yl)-4a-hydroxyoctahydro-2H-chromen-4-yl)carbamate.

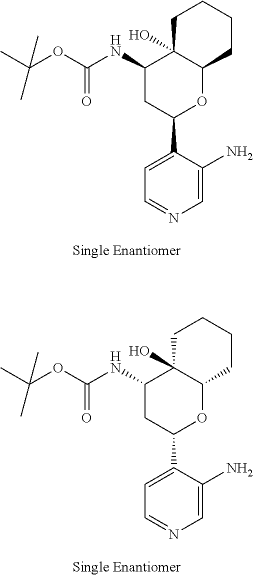 [SMILES: CC(C)(C)OC(=O)N[C@@H]1C[C@H](c2ccncc2N)O[C@@H]2CCCC[C@]12O ; CC(C)(C)OC(=O)N[C@H]1C[C@@H](c2ccncc2N)O[C@H]2CCCC[C@@]12O]

A solution of (+/−)-(2R,4R,4aS,8aR)-4-(benzylamino)-2-(3-nitropyridin-4-yl)octahydro-2H-chromen-4a-ol (1.0 equiv.) in MeOH:EtOAc (4:1, 0.2 M) was degassed with argon for 20 min. At room temperature under an Argon atmosphere, 10% Pearlman's catalyst (Pd hydroxide) (10 mol %) was added and the resulting mixture was evacuated and backfilled with hydrogen gas (three times) and the mixture was then stirred at room temperature under atmospheric partial pressure of hydrogen gas (balloon) for 20 h. The hydrogen gas was then removed by evacuation and the reaction vessel back filled with argon. To the reaction mixture was then added Boc anhydride (2.00 equv.) at RT and the reaction mixture was stirred for 5 h. The reaction mixture was then filtered through celite and the volatiles were removed in vacuo to yield a crude residue. The residue was further purified by flash column chromatography by ISCO Combi-flash Rf system with a Redisep column eluting with 0-10% MeOH/DCM to afford a colourless oil. Purification was completed via chiral HPLC (IPA/heptane=25/75, 20 mL/min, AD column) to yield in order of elution tert-butyl ((2R,4R,4aS,8aR)-2-(3-aminopyridin-4-yl)-4a-hydroxyoctahydro-2H-chromen-4-yl)carbamate (41% yield, 99% ee) and tert-butyl ((2S,4S,4aR,8aS)-2-(3-aminopyridin-4-yl)-4a-hydroxyoctahydro-2H-chromen-4-yl)carbamate (39% yield, 99% ee). LC/MS (m/z): 364.2 (MH⁺), Rₜ=0.55 min. ¹H NMR (CHLOROFORM-d) δ: 1.44 (s, 9H) 1.49-1.77 (m, 6H) 1.87-2.06 (m, 3H) 2.21 (br. s., 1H) 3.47 (br. s., 1H) 3.78-3.89 (m, 1H) 4.16 (br. s., 1H) 4.33 (s, 2H) 4.56 (dd, 1H) 4.78 (d, 1H) 6.91 (d, 1H) 7.95 (d, 1H) 8.03 (s, 1H).

### Synthesis of (2R,3S,6R,4R/S)-4-(benzylamino)-2-cyclopropyl-6-(3-nitropyridin-4-yl)tetrahydro-2H-pyran-3-ol

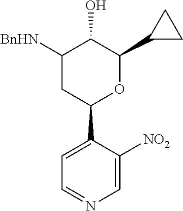

To a solution of (+/−)-(2R,3R,6R)-2-cyclopropyl-3-hydroxy-6-(3-nitropyridin-4-yl)dihydro-2H-pyran-4(3H)-one in MeOH (0.21 M) at RT was added benzyl amine (3.0 equiv.). The reaction mixture was then stirred at RT for 1 h before being cooled to −78° C. followed by the dropwise addition of LiBH₄ (1.10 equiv.). The reaction mixture was then stirred at −78° C. for 4 h. The reaction mixture was then concentrated and diluted with EtOAc. The organic layer was then separated and washed with NaHCO₃ (×2), brine, dried over Na₂SO₄, filtered, and the volatiles were removed in vacuo to yield crude residue. NMR analysis of the unpurified residue indicated a 2:1 mixture of reductive amination diastereoisomers. The unpurified reaction mixture was used in the subsequent transformation without further purification. LC/MS (m/z): 370.3 (MH⁺), Rₜ=0.55 and 0.59 min.

### Synthesis of (+/−)-(2R,3S,4S,6R)—N-benzyl-3-((tert-butyldimethylsilyl)oxy)-2-cyclopropyl-6-(3-nitropyridin-4-yl)tetrahydro-2H-pyran-4-amine and (+/−)-(2R,3S,4R,6R)-4-(benzylamino)-2-cyclopropyl-6-(3-nitropyridin-4-yl)tetrahydro-2H-pyran-3-ol

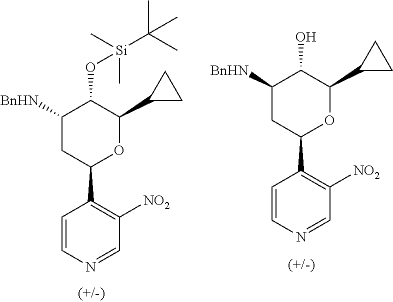

To a solution of (+/−)-(2R,3S,6R,4R/S)-4-(benzylamino)-2-cyclopropyl-6-(3-nitropyridin-4-yl)tetrahydro-2H-pyran-3-ol (1.0 equiv.) in DCM (0.183 M) was added imidazole (10.0 equiv.) followed by TBSCl (3.00 equiv.) at room temperature. The reaction mixture was stirred at RT for 16 h. After 16 h the reaction mixture was concentrated in vacuo then dissolved in EtOAc and washed sequentially with NaHCO₃ then brine dried over Na₂SO₄, filtered then concentrated to yield a crude residue. The residue was further purified by flash column chromatography by ISCO Combi-flash Rf system with a Redisep column eluting with 25-100% EtOAc/heptane to afford in order of elution (+/−)-(2R,3S,4S,6R)—N-benzyl-3-((tert-butyldimethylsilyl)oxy)-2-cyclopropyl-6-(3-nitropyridin-4-yl)tetrahydro-2H-pyran-4-amine (46% yield). LC/MS (m/z): 484.3 (MH⁺), Rₜ=0.98 min. ¹H NMR (CHLOROFORM-d) δ: 0.00 (6H, s), 0.21-0.44 (m, 5H), 0.82 (s, 9H), 1.31-1.41 (m, 1H), 2.33 (d, 1H) overlapping with 2.27 (broad s, 1H), 3.01-3.09 (m, 1H), 3.32-3.40 (m, 1H), 3.54 (d, 1H) 3.59-3.66 (m, 1H), 3.96 (d, 1H), 5.59 (d, 1H), 7.21-7.33 (m, 5H), 7.69 (s, 1H), 8.67 (dd, 1H), 9.04 (s, 1H) followed by (+/−)-2R,3S,4R,6R)-4-(benzylamino)-2-cyclopropyl-6-(3-nitropyridin-4-yl)tetrahydro-2H-pyran-3-ol carbamate (22% yield). LC/MS (m/z): 370.1 (MH⁺), Rₜ=0.60 min. ¹H NMR (CHLOROFORM-d) δ: 0.28-0.42 (m, 1H), 0.43-0.55 (m, 2H), 0.55-0.67 (m, 1H), 0.97-1.17 (m, 2H), 1.30 (m, 1H), 2.49-2.61 (m, 1H), 2.78-2.94 (m, 2H), 3.35 (t, 1H), 3.75 (d, 1H), 3.92 (d, 1H), 4.10 (dd, 1H), 5.04 (d, 1H), 7.13 7.38 (m, 5H), 7.74 (d, 1H), 8.79 (d, 1H), 9.16 (s, 1H).

### Synthesis of (+/−)-(2R,3S,4R,6R)—N-benzyl-2-cyclopropyl-6-(3-nitropyridin-4-yl)-3-((triethylsilyl)oxy)tetrahydro-2H-pyran-4-amine

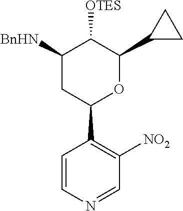

To a solution of (+/−)-(2R,3S,4R,6R)-4-(benzylamino)-2-cyclopropyl-6-(3-nitropyridin-4-yl)tetrahydro-2H-pyran-3-ol (1.0 equiv.) and triethylamine (2.5 equiv.) in DCM (0.12 M) cooled to 0° C. was added Triethylsilyl trifluoromethanesulfonate (2.4 equiv.) dropwise over five minutes. The resulting mixture was stirred at 0° C. for 2 h. The reaction mixture was then quenched with NaHCO₃ and diluted with EtOAc. The organic layer was separated and washed with NaHCO₃ and brine then dried over MgSO₄, filtered and concentrated in vacuo to yield the desired product (+/−)-(2R,3S,4R,6R)—N-benzyl-2-cyclopropyl-6-(3-nitropyridin-4-yl)-3-((triethylsilyl)oxy)tetrahydro-2H-pyran-4-amine as a colourless oil which was used in the subsequent transformation without further purification. LC/MS (m/z): 484.3 (MH⁺), Rₜ=1.01 min.

### Synthesis of tert-butyl ((2R,3S,4R,6R)-6-(3-aminopyridin-4-yl)-2-cyclopropyl-3-((triethylsilyl)oxy)tetrahydro-2H-pyran-4-yl)carbamate and tert-butyl ((2S,3R,4S,6S)-6-(3-aminopyridin-4-yl)-2-cyclopropyl-3-((triethylsilyl)oxy)tetrahydro-2H-pyran-4-yl)carbamate

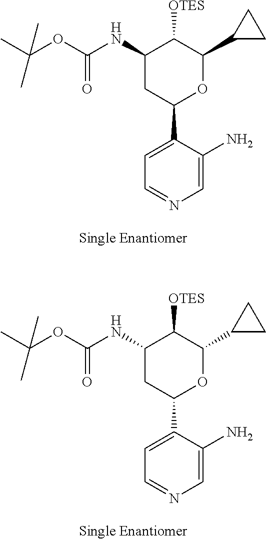

A solution of (+/−)-(2R,3S,4R,6R)—N-benzyl-2-cyclopropyl-6-(3-nitropyridin-4-yl)-3-((triethylsilyl)oxy)tetrahydro-2H-pyran-4-amine (1.0 equiv.) in MeOH:EtOAc (1:1, 0.1 M) was degassed with argon for 20 min. At room temperature under an Argon atmosphere, 10% Pearlman's catalyst (Pd hydroxide) (10 mol %) was added and the resulting mixture was evacuated and backfilled with hydrogen gas (three times) and the mixture was then stirred at room temperature under atmospheric partial pressure of hydrogen gas (balloon) overnight. The hydrogen gas was then removed by evacuation and the reaction vessel back filled with argon. To the reaction mixture was then added Boc anhydride (1.00 equv.) at RT and the reaction mixture was stirred for 16 h. The reaction mixture was then filtered through celite and the volatiles were removed in vacuo to yield a crude residue. The residue was further purified by flash column chromatography by ISCO Combi-flash Rf system with a Redisep column eluting with 0-80% EtOAc/heptane to afford a colourless oil. Purification was completed via chiral HPLC (IPA/heptane=10/90, 20 mL/min, AD-H column) to yield in order of elution tert-butyl ((2R,4R,4aS,8aR)-2-(3-aminopyridin-4-yl)-4a-hydroxyoctahydro-2H-chromen-4-yl)carbamate (29% yield, 99% ee) and tert-butyl ((2S,4S,4aR,8aS)-2-(3-aminopyridin-4-yl)-4a-hydroxyoctahydro-2H-chromen-4-yl)carbamate (31% yield, 99% ee). LC/MS (m/z): 364.2 (MH⁺), Rₜ=0.72 min. ¹H NMR (CHLOROFORM-d) δ: 0.27-0.37 (m, 1H), 0.47 (m, 1H), 0.51-0.64 (m, 2H), 0.69 (q, 6H), 0.91-1.12 (m, 10H), 1.42-1.51 (s, 9H), 1.86 (dd, 1H), 2.23-2.33 (m, 1H), 2.99 (m, 1H), 3.41 (m, 1H), 3.64-3.77 (m, 1H), 4.15-4.22 (m, 2H), 4.38-4.48 (m, 2H), 6.92 (d, 1H), 7.96 (d, 1H), 8.03 (s, 1H).

### Synthesis of diethyl (3-oxobutan-2-yl)phosphonate

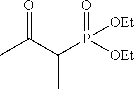

To a suspension of sodium iodide (1.0 equiv.) in MeCN (1.34 M) at RT was added dropwise 3-chlorobutan-2-one (1.0 equiv.) The resulting mixture was then heated to reflux (83° C.) before the dropwise addition of triethyl phosphite (1.00 equiv.) followed by continued heating at 83° C. for 14 h. The reaction mixture was then filtered through a pad of silica gel and concentrated to yield a red oil. The oil was further purified by bulb to bulb distillation under reduced pressure at 170-180° C. to afford the desired product diethyl (3-oxobutan-2-yl)phosphonate as a colourless oil (yield=65%, 80% purity contaminated with triethyl phosphite). LC/MS (m/z): 209.1 (MH⁺), Rₜ=0.48 min. ¹H NMR (400 MHz, CHLOROFORM-d) δ ppm: 1.20-1.36 (m, 7H) 2.28 (d, 3H) 3.06-3.25 (m, 1H) 3.97-4.22 (m, 6H)

### Synthesis of (E)-4-cyclopropyl-3-methylbut-3-en-2-one

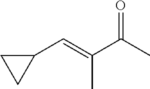

To a solution of NaH (2.00 equiv., 60% suspended in mineral oil washed with pentanes) in THF (0.243 M) at 0° C. was added diethyl (3-oxobutan-2-yl)phosphonate (2.0 equiv.) dropwise. The resulting solution was stirred at 0° C. for 1 h, before the addition of a solution of cyclopropanecarbaldehyde (1.00 equiv.) in THF (0.86 M) dropwise over 10 min. The reaction mixture was then allowed to warm to RT with continued stirring for 4 h. The reaction mixture was then quenched with NH₄Cl, the aqueous layer was separated and extracted with Et₂O. The combined organics were then dried over Na₂SO₄, filtered and concentrated in vacuo to yield a colorless oil. The oil was further purified by flash column chromatography by ISCO Combi-flash Rf system with a Redisep column eluting with 0-20% Et₂O/pentanes to afford the desired product (E)-4-cyclopropyl-3-methylbut-3-en-2-one as a solution in 1:1 Et2O:pentane (0.09 M). LC/MS (m/z): 125.0 (MH⁺), Rₜ=0.67 min. ¹H NMR (400 MHz, CHLOROFORM-d) δ ppm 0.57-0.68 (m, 2H) 0.96-1.05 (m, 2H) 1.63-1.74 (m, 1H) 2.03 (s, 3H) 2.23 (s, 3H) 5.94 (d, 1H).

### Method 7

### Synthesis of (E)-((4-cyclopropyl-3-methylbuta-1,3-dien-2-yl)oxy)triethylsilane

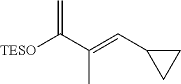

To a solution of (E)-4-cyclopropyl-3-methylbut-3-en-2-one (1.00 equiv.) and triethylamine (2.00 equiv.) in heptane:Et₂O (1:1 0.08 M) cooled to 0° C. was added triethylsilyl trifluoromethanesulfonate (1.34 equiv.) dropwise over five minutes. The resulting mixture was stirred at 0° C. for 4 h. The reaction mixture was then quenched with NaHCO₃, the aqueous layer was separated and extracted with Et₂O. The combined organics were then dried over MgSO₄, filtered and concentrated in vacuo to yield the desired product (E)-((4-cyclopropyl-3-methylbuta-1,3-dien-2-yl)oxy)triethylsilane as a colourless oil (yield=82%) which was used in the Hetero-Diels Alder reaction without further purification.

### Method 8

### Synthesis of cis (+/−)-4-((2R,6R)-6-cyclopropyl-4-((triethylsilyl)oxy)-3,6-dihydro-2H-pyran-2-yl)-3-nitropyridine

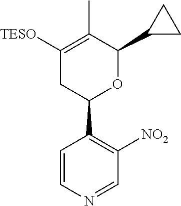

A solution of 3-nitroisonicotinaldehyde (1.20 equiv.), (E)-((4-cyclopropyl-3-methylbuta-1,3-dien-2-yl)oxy)triethylsilane (1.00 equiv.), and tris(6,6,7,7,8,8,8-heptafluoro-2,2-dimethyl-3,5-octanedionato) europium (0.05 equiv.) were dissolved in CHCl₃ (0.18 M) and stirred in a flame-dried round-bottom flask at 60° C. under an atmosphere of nitrogen for 1 h before being stirred for a further 3 h at RT. After this time the reaction mixture was cooled to room temperature and concentrated in vacuo to yield yellow oil. The oil was further purified by flash column chromatography by ISCO Combi-flash Rf system with a Redisep column eluting with 0-30% Et₂O/heptanes with 1% Et₃N to afford the desired product cis (+/−)-4-((2R,6R)-6-cyclopropyl-5-methyl-4-((triethylsilyl)oxy)-3,6-dihydro-2H-pyran-2-yl)-3-nitropyridine as a red oil (93% yield over three steps). LC/MS (m/z): 391.1 (MH⁺), Rₜ=1.39 min. ¹H NMR (400 MHz, CHLOROFORM-d) δ ppm 0.35-0.50 (m, 2H) 0.55 (dd, 1H) 0.63-0.77 (m, 7H) 0.90-1.08 (m, 10H) 1.71-1.81 (m, 3H) 2.30-2.47 (m, 1H) 2.49-2.65 (m, 1H) 3.51 (d, 1H) 5.25 (dd, 1H) 7.93 (d, 1H) 9.22 (s, 1H), 9.57 (s, 1H).

### Method 9

### Synthesis of (+/−)-(2R,3R,6R)-2-cyclopropyl-3-hydroxy-3-methyl-6-(3-nitropyridin-4-yl)dihydro-2H-pyran-4(3H)-one

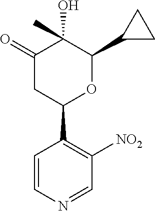 [SMILES: C[C@]1(O)C(=O)C[C@H](c2ccncc2[N+](=O)[O-])O[C@@H]1C1CC1]

To a solution of cis-(+/−)-4-((2R,6R)-6-cyclopropyl-5-methyl-4-((triethylsilyl)oxy)-3,6-dihydro-2H-pyran-2-yl)-3-nitropyridine (1.0 equiv.) in EtOAc:water 1:1 (0.15 M) was added acetone (15.0 equiv.), NaHCO₃ (7.50 equiv.) at RT. To the resulting solution was added a solution of oxone (1.40 equv.) in water (0.42 M) dropwise by addition funnel taking care to keep the internal reaction temperature below 20° C. The reaction mixture was stirred at RT for 5 h before being quenched with cyclohexene and diluted with EtOAc and brine. The organic layer was then separated, dried over Na₂SO₄, filtered and the volatiles were removed in vacuo. The residue was taken up in THF (0.32 M) at room temperature and acidified with 4 M HCl (1.5 equiv.) the reaction mixture was then stirred for 1 h at RT. The reaction mixture was then quenched with NaHCO₃ (sat.). The aqueous layer was separated and extracted with EtOAc. The combined organics were then dried over Na₂SO₄, filtered and concentrated in vacuo to yield a colourless oil. The oil was further purified by flash column chromatography by ISCO Combi-flash Rf system with a Redisep column eluting with 0-50% EtOAc/heptanes to afford as a single diastereoisomer the desired product (+/−)-(2R,3R,6R)-2-cyclopropyl-3-hydroxy-3-methyl-6-(3-nitropyridin-4-yl)dihydro-2H-pyran-4(3H)-one as a colourless oil (54% yield). LC/MS (m/z): 293.1 (MH⁺), Rₜ=0.63 min. ¹H NMR (400 MHz, CHLOROFORM-d) δ ppm 0.46-0.56 (m, 2H) 0.57-0.66 (m, 2H) 1.21 (m, 1H) 1.56 (s, 3H) 2.74 (dd, 1H) 3.05 (dd, 1H) 3.11 (d, 1H) 3.89 (s, 1H) 5.27 (dd, 1H) 7.81 (d, J=5.03 Hz, 1H) 8.91 (d, J=5.32 Hz, 1H) 9.22 (s, 1H).

### Synthesis of (+/−)-(2R,3S,4R,6R)-2-cyclopropyl-3-methyl-6-(3-nitropyridin-4-yl)tetrahydro-2H-pyran-3,4-diol

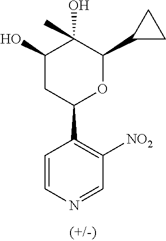 [SMILES: C[C@]1(O)[C@H](O)C[C@H](c2ccncc2[N+](=O)[O-])O[C@@H]1C1CC1]

To a solution of (+/−)-(2R,3R,6R)-2-cyclopropyl-3-hydroxy-3-methyl-6-(3-nitropyridin-4-yl)dihydro-2H-pyran-4(3H)-one (1.0 equiv.) in EtOH (0.20 M) at 0° C. was added sodium borohydride (1.0 equiv.). The reaction mixture was allowed to stir for 30 min warming to room temperature. The reaction mixture was then concentrated and pardoned between water and EtOAc. The aqueous layer was then separated and extracted with EtOAc (×2) the combined organics were then washed with brine, dried over Na₂SO₄, filtered, and the volatiles were removed in vacuo to yield (+/−)-(2R,3S,4R,6R)-2-cyclopropyl-3-methyl-6-(3-nitropyridin-4-yl)tetrahydro-2H-pyran-3,4-diol as a colourless oil (yield=99%) which was used in the subsequent reaction without further purification. LC/MS (m/z): 295.1 (MH⁺), Rₜ=0.52 min.

### Synthesis of (+/−)-(2R,3S,4R,6R)-2-cyclopropyl-6-(3-nitropyridin-4-yl)tetrahydro-2H-pyran-3,4-diyl diacetate

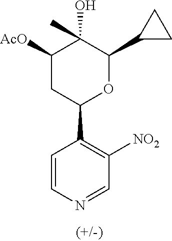 [SMILES: CC(=O)O[C@@H]1C[C@H](c2ccncc2[N+](=O)[O-])O[C@H](C2CC2)[C@@]1(C)O]

To a solution of (+/−)-(2R,3S,4R,6R)-2-cyclopropyl-3-methyl-6-(3-nitropyridin-4-yl)tetrahydro-2H-pyran-3,4-diol (1.0 equiv.) in pyridine (0.182 M) at room temperature was added acetic anhydride (6.0 equiv.). The reaction mixture was stirred for 7 hr at room temperature. The reaction was quenched with water and the product was extracted in EtOAc and washed with brine. The organics were dried over MgSO₄, filtered, and volatiles were removed in vacuo to yield (+/−)-(2R,3S,4R,6R)-2-cyclopropyl-3-hydroxy-3-methyl-6-(3-nitropyridin-4-yl)tetrahydro-2H-pyran-4-yl acetate as a colourless oil (unpurified mass recovery=33%). The oil was used in the subsequent reaction without further purification. LC/MS (m/z): 337.1 (MH⁺), Rₜ=0.70 min.

### Synthesis of (2R,3S,4R,6R)-6-(3-aminopyridin-4-yl)-2-cyclopropyl-3-hydroxy-3-methyltetrahydro-2H-pyran-4-yl acetate and (2S,3R,4S,6S)-6-(3-aminopyridin-4-yl)-2-cyclopropyl-3-hydroxy-3-methyltetrahydro-2H-pyran-4-yl acetate

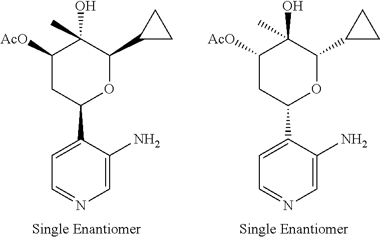 [SMILES: CC(=O)O[C@@H]1C[C@H](c2ccncc2N)O[C@H](C2CC2)[C@@]1(C)O ; CC(=O)O[C@H]1C[C@@H](c2ccncc2N)O[C@@H](C2CC2)[C@]1(C)O]

To a solution of (+/−)-(2R,3S,4R,6R)-2-cyclopropyl-3-hydroxy-3-methyl-6-(3-nitropyridin-4-yl)tetrahydro-2H-pyran-4-yl acetate (1.0 equiv.) in AcOH (0.178 M) at RT was added Iron powder (10.0 equiv.). The reaction mixture was stirred at RT for 2 h. After this time the reaction mixture was concentrated to dryness diluted with EtOAc and NaHCO₃. The organic layer was then separated and washed with NaHCO₃, brine, dried over Na₂SO₄, filtered, and volatiles were removed in vacuo to yield a colourless oil. The oil was further purified by flash column chromatography by ISCO Combi-flash Rf system with a Redisep column eluting with 0-100% EtOAc/heptanes to afford a colourless oil. Further chiral separation and purification was completed via chiral HPLC (heptane/EtOH=85/15, 20 mL/min, AD column) to yield in order of elution (2R,3S,4R,6R)-6-(3-aminopyridin-4-yl)-2-cyclopropyl-3-hydroxy-3-methyltetrahydro-2H-pyran-4-yl acetate (37% yield, 99% ee) and (2S,3R,4S,6S)-6-(3-aminopyridin-4-yl)-2-cyclopropyl-3-hydroxy-3-methyltetrahydro-2H-pyran-4-yl acetate (40% yield, 99% ee). LC/MS (m/z): 307.1 (MH⁺), Rₜ=0.42 min. ¹H NMR (400 MHz, CHLOROFORM-d) δ ppm 0.36 (m, 1H) 0.47-0.69 (m, 3H) 1.03-1.15 (m, 1H), 1.42 (s, 3H), 2.01-2.19 (m, 2H) overlapping with 2.14 (s, 3H), 2.88 (d, 1H) 4.24 (br. s., 2H) 4.52 (dd, 1H) 4.99 (dd, 1H) 6.93 (d, 1H) 7.99 (d, 1H) 8.06 (s, 1H).

### Synthesis of (E)-4,4-dimethylpent-2-enoic acid

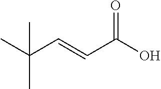

To a flame dried flask in an inert argon atmosphere was added Ni(COD)₂ (0.91 equiv.) followed by THF (0.126 M), the resulting flask was evacuated and backfilled with argon gas. The reaction mixture was then removed from the inert atmosphere and cooled to 0° C. The reaction vessel was then evacuated and backfilled with CO₂ gas (three times) and placed under an atmospheric partial pressure of CO₂ gas (balloon) followed by the dropwise addition of a solution of 3,3-dimethylbut-1-yne in THF (0.126 M) over 90 mins. The reaction mixture was then quenched by the dropwise addition of 0.5 N HCl (0.77 eq. of initial volume of THF). The reaction mixture was transferred to a separation funnel and addition 1 M HCl (0.77 eq. of initial volume of THF) was added to acidify the solution followed by the addition of DCM. The aqueous layer was separated and extracted with DCM (×2) and the combined organics were washed with brine. The organic layer was then further extracted with 0.1 M NaOH (×3). The aqueous layer was then acidified with 1M HCl and extracted with DCM (×3). The combined organics were washed with brine, dried over Na₂SO₄. filtered and concentrated in vacuo to afford the desired product (E)-4-cyclopropyl-3-methylbut-3-en-2-one as a white solid (yield=78%). LC/MS (m/z): 128.9 (MH⁺), Rₜ=0.64 min. ¹H NMR (400 MHz, CHLOROFORM-d) δ ppm 1.10 (s, 9H), 5.75 (d, 1H), 7.18 (d, 1H).

### Synthesis of (E)-5,5-dimethylhex-3-en-2-one

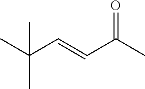

To a solution of (E)-4,4-dimethylpent-2-enoic acid (1.00 equiv.) in THF (0.08 M) cooled to −78° C. was added MeLi (2.00 equiv., 1.6 M in Et₂O) added rapidly. The resulting mixture was stirred at −78 for 1 h before warming to 0° C. over an additional 1 h. The reaction mixture was then quenched by cannula transfer to a 0.12N HCl (0.5 eq. of initial THF volume) followed by dilution with Et₂O. The aqueous layer was separated and acidified further with 1M HCl then extracted with DCM (×2). The combined organics were then washed with NaHCO₃, brine, dried over Na₂SO₄, filtered and concentrated in vacuo to yield the desired product (E)-5,5-dimethylhex-3-en-2-one as a solution in DCM which was used in the subsequent transformation without further purification. LC/MS (m/z): 126.9 (MH⁺), Rₜ=0.73 min. ¹H NMR (400 MHz, CHLOROFORM-d) δ ppm 1.10 (s, 9H), 2.26 (s, 3H), 6.00 (d, 1H), 6.79 (d, 1H).

### Synthesis of (E)-((5,5-dimethylhexa-1,3-dien-2-yl)oxy)triethylsilane

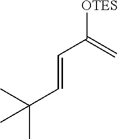

To a solution of (E)-5,5-dimethylhex-3-en-2-one (1.00 equiv.) in DCM (2.4 M) at RT was added DBU (2,3,4,6,7,8,9,10-octahydropyrimido[1,2-a]azepine, 1.00 equiv.) followed triethylsilylchlroide (1.34 equiv.). The resulting mixture was stirred at RT for 15 min before being heated to 39° C. for 4 h. The reaction mixture was then quenched with NaHCO₃, the aqueous layer was separated and extracted with DCM. The combined organics were washed with brine then dried over MgSO₄, filtered and concentrated in vacuo to yield the desired product (E)-((4-cyclopropyl-3-methylbuta-1,3-dien-2-yl)oxy)triethylsilane as a colourless oil which was used in the Hetero-Diels Alder reaction without further purification.

### Synthesis of cis (+/−)-4-((2R,6R)-6-(tert-butyl)-5-methyl-4-((triethylsilyl)oxy)-3,6-dihydro-2H-pyran-2-yl)-3-nitropyridine

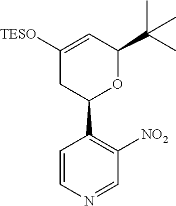

A solution of 3-nitroisonicotinaldehyde (1.40 equiv.), (E)-((5,5-dimethylhexa-1,3-dien-2-yl)oxy)triethylsilane (1.00 equiv.), and tris(6,6,7,7,8,8,8-heptafluoro-2,2-dimethyl-3,5-octanedionato) europium (0.05 equiv.) were dissolved in CHCl₃ (0.2 M) and stirred in a flame-dried round-bottom flask at 60° C. under an atmosphere of nitrogen for 3 h before being stirred overnight at RT. After this time the reaction mixture was cooled to room temperature and concentrated in vacuo to yield yellow oil. The oil was further purified by flash column chromatography by ISCO Combi-flash Rf system with a Redisep column eluting with 0-40% Et₂O/heptanes with 1% Et₃N to afford the desired product cis (+/−)-4-((2R,6S)-6-(tert-butyl)-4-((triethylsilyl)oxy)-3,6-dihydro-2H-pyran-2-yl)-3-nitropyridine as a colourless oil (51% yield). LC/MS (m/z): 393.3 (MH⁺), Rₜ=1.45 min. ¹H NMR (400 MHz, CHLOROFORM-d) δ ppm 10.35 (br. s., 1H), 9.92 (br. s., 1H), 8.16 (d, 1H), 5.52 (dd, 1H), 5.00-5.10 (m, 1H), 3.98-4.13 (m, 1H), 2.58-2.73 (m, 1H), 2.30-2.46 (m, 1H), 0.92-1.12 (m, 16H), 0.65-0.82 (m, 6H).

### Method 10

### Synthesis of (+/−)-(2R,3R,6R)-2-(tert-butyl)-3-hydroxy-3-methyl-6-(3-nitropyridin-4-yl)dihydro-2H-pyran-4(3H)-one

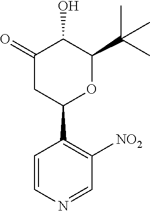 [SMILES: CC(C)(C)[C@H]1O[C@@H](c2ccncc2[N+](=O)[O-])CC(=O)[C@@H]1O]

To a solution of cis-(+/−)-4-((2R,6S)-6-(tert-butyl)-4-((triethylsilyl)oxy)-3,6-dihydro-2H-pyran-2-yl)-3-nitropyridine (1.0 equiv.) in DCM (0.24 M) cooled to 0° C. was added 3,3-dimethyldioxirane as a solution in acetone (0.1M solution, 1.00 equiv.) and allowed to stir for 2 h. To the reaction was added 5 mL of cyclohexene; the reaction mixture was stirred for 10 mins and the volatiles were removed in vacuo. The residue was taken up in THF (0.05 M) at room temperature and acidified with 1M HCl (5.0 equiv.) the reaction stirred for 1 h. The solution was basified with 1 M NaOH to ˜pH=9. The product was extracted in EtOAc washed with brine, dried over MgSO₄, filtered and the volatiles were removed in vacuo. The oil was further purified by flash column chromatography by ISCO Combi-flash Rf system with a Redisep column eluting with 0-40% EtOAc/heptanes to afford as a single diastereoisomer the desired product (+/−)-(2R,3R,6R)-2-(tert-butyl)-3-hydroxy-6-(3-nitropyridin-4-yl)dihydro-2H-pyran-4(3H)-one as a colourless oil (78% yield). LC/MS (m/z): 295.0 (MH⁺), Rₜ=0.77 min. ¹H NMR (400 MHz, CHLOROFORM-d) δ ppm 9.25 (s, 1H), 8.91 (d, 1H), 7.86 (d, 1H), 5.33 (dd, 1H), 4.25 (dd, 1H), 3.78 (m, 1H), 3.25 (d, 1H), 3.17 (dd, 1H), 2.60 (dd, 1H), 1.12 (s, 9H).

### Method 11

### Synthesis of (+/−)-(2R,3S,4S,6R)-4-(benzylamino)-2-(tert-butyl)-6-(3-nitropyridin-4-yl)tetrahydro-2H-pyran-3-ol and (+/−)-(2R,3S,4R,6R)-4-(benzylamino)-2-(tert-butyl)-6-(3-nitropyridin-4-yl)tetrahydro-2H-pyran-3-ol

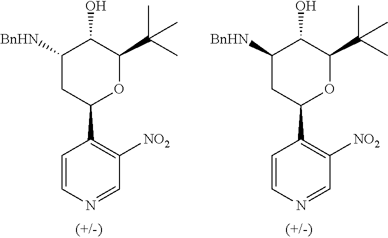

To a solution of (+/−)-(2R,3R,6R)-2-(tert-butyl)-3-hydroxy-6-(3-nitropyridin-4-yl)dihydro-2H-pyran-4(3H)-one (1.0 equiv.) in MeOH (0.28 M) at RT was added benzyl amine (3.0 equiv.). The reaction mixture was then stirred at RT for 18 h before being cooled to −78° C. followed by the dropwise addition of LiBH₄ (1.10 equiv.). The reaction mixture was then stirred at −78° C. for 2 h before being warmed to 0° C. over 10 min. The reaction mixture was then quenched with NaHCO₃. The aqueous layer was then separated and extracted with EtOAc. The combined organics were washed with brine, dried over Na₂SO₄, filtered, and the volatiles were removed in vacuo to yield crude residue. The oil was further purified by flash column chromatography by ISCO Combi-flash Rf system with a Redisep column eluting with 0-40-75% EtOAc/heptanes to afford (+/−)-(2R,3S,4S,6R)-4-(benzylamino)-2-(tert-butyl)-6-(3-nitropyridin-4-yl)tetrahydro-2H-pyran-3-ol in 30% yield, LC/MS (m/z): 386.0 (MH⁺), Rₜ=0.71 min, ¹H NMR (400 MHz, CHLOROFORM-d) δ ppm 1.01-1.09 (m, 9H), 1.51 (s, 1H), 2.45 (d, J=13.69 Hz, 1H), 3.06-3.15 (m, 2H), 3.73 (d, J=12.52 Hz, 2H), 4.08 (d, J=12.52 Hz, 1H), 5.26 (dd, J=10.63, 2.18 Hz, 1H), 7.29-7.34 (m, 1H), 7.34-7.40 (m, 2H), 7.41-7.45 (m, 2H), 7.80 (d, J=5.24 Hz, 1H), 8.82 (d, J=4.95 Hz, 1H), 9.24 (s, 1H); (+/−)-(2R,3S,4R,6R)-4-(benzylamino)-2-(tert-butyl)-6-(3-nitropyridin-4-yl)tetrahydro-2H-pyran-3-ol as a colourless oil in 18% yield, LC/MS (m/z): 386.2 (MH⁺), Rₜ=0.72 min, ¹H NMR (400 MHz, CHLOROFORM-d) δ ppm 1.06 (s, 9H), 1.15-1.24 (m, 1H), 2.56-2.61 (m, 1H), 2.78-2.84 (m, 1H), 3.10 (d, 1H), 3.31 (t, 1H), 3.40 (br.s, 1H), 3.75 (dd, 1H), 3.94 (dd, 1H), 4.12 (dd, 1H), 5.08 (d, 1H), 7.28-7.36 (m, 5H), 7.76 (d, 1H) 8.81 (d, 1H) 9.20 (s, 1H).

### Method 12

### Synthesis of tert-butyl ((2S,3R,4S,6S)-6-(3-aminopyridin-4-yl)-2-(tert-butyl)-3-hydroxytetrahydro-2H-pyran-4-yl)carbamate and tert-butyl ((2R,3S,4R,6R)-6-(3-aminopyridin-4-yl)-2-(tert-butyl)-3-hydroxytetrahydro-2H-pyran-4-yl)carbamate.

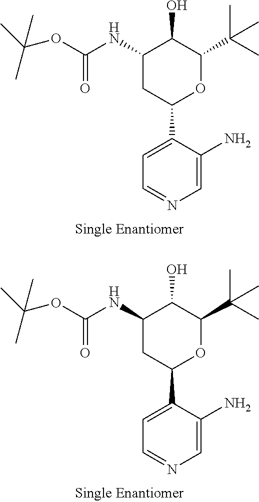

A solution of (+/−)-(2R,3S,4R,6R)-4-(benzylamino)-2-(tert-butyl)-6-(3-nitropyridin-4-yl)tetrahydro-2H-pyran-3-ol (1.0 equiv.) in MeOH (0.15 M) was degassed with argon for 20 min. At room temperature under an Argon atmosphere, 10% Pearlman's catalyst (Pd hydroxide) (20 mol %) was added and the resulting mixture was evacuated and backfilled with hydrogen gas (three times) and the mixture was then stirred at room temperature under atmospheric partial pressure of hydrogen gas (balloon) overnight. The hydrogen gas was then removed by evacuation and the reaction vessel back filled with argon. To the reaction mixture was then added Boc anhydride (1.00 equv.) at RT and the reaction mixture was stirred for 16 h. The reaction mixture was then filtered through celite and the volatiles were removed in vacuo to yield a crude residue. The residue was further purified by flash column chromatography by ISCO Combi-flash Rf system with a Redisep column eluting with 0-80% EtOAc/heptane to afford a colourless oil. Purification was completed via chiral HPLC (IPA/heptane=10/90, 20 mL/min, AD-H column) to yield in order of elution tert-butyl ((2S,3R,4S,6S)-6-(3-aminopyridin-4-yl)-2-(tert-butyl)-3-hydroxytetrahydro-2H-pyran-4-yl)carbamate (35% yield, 99% ee) and tert-butyl ((2R,3S,4R,6R)-6-(3-aminopyridin-4-yl)-2-(tert-butyl)-3-hydroxytetrahydro-2H-pyran-4-yl)carbamate (26% yield, 99% ee). LC/MS (m/z): 366.1 (MH⁺), Rₜ=0.64 min. ¹H NMR (400 MHz, CHLOROFORM-d) δ ppm 1.06 (s, 9H), 1.46 (s, 9H), 1.85 (d, J=12.13 Hz, 1H), 2.09-2.19 (m, 1H), 3.09 (d, J=9.00 Hz, 1H), 3.46 (d, J=7.83 Hz, 2H), 3.73-3.87 (m, 1H), 4.19 (s, 2H), 4.44 (dd, J=11.54, 1.76 Hz, 1H), 4.69 (br. s., 1H), 6.92 (d, J=4.70 Hz, 1H), 7.98 (d, J=5.09 Hz, 1H), 8.05 (s, 1H).

### Synthesis of tert-butyl ((2R,3S,4S,6R)-6-(3-aminopyridin-4-yl)-2-(tert-butyl)-3-hydroxytetrahydro-2H-pyran-4-yl)carbamate and tert-butyl ((2S,3R,4R,6S)-6-(3-aminopyridin-4-yl)-2-(tert-butyl)-3-hydroxytetrahydro-2H-pyran-4-yl)carbamate

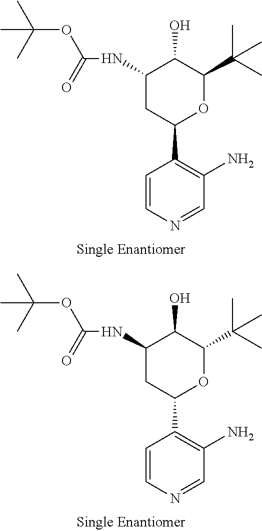

Method 12 was followed using (+/−)-(2R,3S,4S,6R)-4-(benzylamino)-2-(tert-butyl)-6-(3-nitropyridin-4-yl)tetrahydro-2H-pyran-3-ol (1.0 equiv.), 20% Pearlman's catalyst (Pd hydroxide) (20 mol %) and Boc anhydride (1.1 equiv.) in MeOH (0.14 M). Purification was completed via SFC (MeOH+0.1% DEA=20%, 15 mL/min, AD column) to yield in order of elution tert-butyl ((2R,3S,4S,6R)-6-(3-aminopyridin-4-yl)-2-(tert-butyl)-3-hydroxytetrahydro-2H-pyran-4-yl)carbamate (48% yield, 99% ee) and tert-butyl ((2S,3R,4R,6S)-6-(3-aminopyridin-4-yl)-2-(tert-butyl)-3-hydroxytetrahydro-2H-pyran-4-yl)carbamate (48% yield, 99% ee). LC/MS (m/z): 366.1 (MH⁺), Rₜ=0.65 min.

### Synthesis of (2R,3S,4R/S,6R)-4-(benzylamino)-2-cyclopropyl-3-methyl-6-(3-nitropyridin-4-yl)tetrahydro-2H-pyran-3-ol

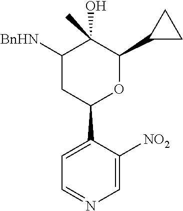

To a solution of (+/−)-(2R,3R,6R)-2-cyclopropyl-3-hydroxy-3-methyl-6-(3-nitropyridin-4-yl)dihydro-2H-pyran-4(3H)-one in MeOH (0.15 M) at RT was added benzyl amine (3.0 equiv.). The reaction mixture was then stirred at RT for 16 h before being cooled to −78° C. followed by the dropwise addition of LiBH₄ (1.10 equiv.). The reaction mixture was then stirred at −78° C. for 1 h before being warmed to RT and stirred for a further 3 h. The reaction mixture was then concentrated and diluted with EtOAc. The organic layer was then separated and washed with NaHCO₃ (×2), brine, dried over Na₂SO₄, filtered, and the volatiles were removed in vacuo to yield crude residue. The unpurified reaction mixture was used in the subsequent transformation without further purification. LC/MS (m/z): 384.3 (MH⁺), Rₜ=0.55 min.

### Synthesis of (tert-butyl ((2R,3S,4R,6R)-6-(3-aminopyridin-4-yl)-2-cyclopropyl-3-hydroxy-3-methyltetrahydro-2H-pyran-4-yl)carbamate and tert-butyl ((2S,3R,4S,6S)-6-(3-aminopyridin-4-yl)-2-cyclopropyl-3-hydroxy-3-methyltetrahydro-2H-pyran-4-yl)carbamate

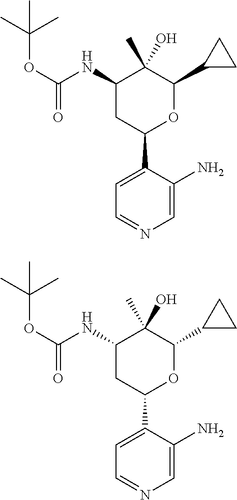 [SMILES: CC(C)(C)OC(=O)N[C@@H]1C[C@H](c2ccncc2N)O[C@H](C2CC2)[C@@]1(C)O ; CC(C)(C)OC(=O)N[C@H]1C[C@@H](c2ccncc2N)O[C@@H](C2CC2)[C@]1(C)O]

A solution of (+/−)-(2R,3S,6R)-4-(benzylamino)-2-cyclopropyl-3-methyl-6-(3-nitropyridin-4-yl)tetrahydro-2H-pyran-3-ol (1.0 equiv.) in MeOH (0.2 M) was degassed with argon for 20 min. At room temperature under an Argon atmosphere, 10% Pearlman's catalyst (Pd hydroxide) (20 mol %) was added and the resulting mixture was evacuated and backfilled with hydrogen gas (three times) and the mixture was then stirred at room temperature under atmospheric partial pressure of hydrogen gas (balloon) for 17 h. The hydrogen gas was then removed by evacuation and the reaction vessel back filled with argon. To the reaction mixture was then added Boc anhydride (2.60 equv.) at RT and the reaction mixture was stirred for 4 h. The reaction mixture was then filtered through celite and the volatiles were removed in vacuo to yield a crude residue. The residue was further purified by flash column chromatography by ISCO Combi-flash Rf system with a Redisep column eluting with 0-45-55% acetone/heptane to afford a colourless oil. Purification was completed via chiral HPLC (IPA/heptane=15/85, 20 mL/min, AD column) to yield in order of elution tert-butyl (tert-butyl ((2R,3S,4R,6R)-6-(3-aminopyridin-4-yl)-2-cyclopropyl-3-hydroxy-3-methyltetrahydro-2H-pyran-4-yl)carbamate (17% yield, 99% ee) and tert-butyl ((2S,3R,4S,6S)-6-(3-aminopyridin-4-yl)-2-cyclopropyl-3-hydroxy-3-methyltetrahydro-2H-pyran-4-yl)carbamate. (17% yield, 99% ee) LC/MS (m/z): 364.2 (MH⁺), Rₜ=0.54 min. ¹H NMR (CHLOROFORM-d) δ: 0.33 (d, 1H) 0.53 (t, 2H) 0.62 (d, 1H) 1.11 (d, 1H) 1.30 (s, 3H) 1.45-1.50 (m, 9H) 1.89 (d, 1H) 1.98-2.08 (m, 1H) 2.92 (d, 1H) 3.79-3.90 (m, 1H) 4.51 (dd, 1H) 6.90 (d, 1H) 7.99 (d, 1H) 8.06 (s, 1H).

### Synthesis of (+/−)-(2R,3S,4R,6R)-4-(benzylamino)-2-ethyl-3-methyl-6-(3-nitropyridin-4-yl)tetrahydro-2H-pyran-3-ol

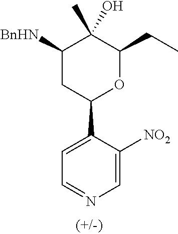

Method 11 was followed using (+/−)-(2R,3R,6S)-2-ethyl-3-hydroxy-3-methyl-6-(3-nitropyridin-4-yl)dihydro-2H-pyran-4(3H)-one (1.0 equiv.) and benzyl amine (3.0 equiv.) and LiBH₄ (1.10 equiv.) in MeOH (0.17 M) to give (+/−)-(2R,3S,4R,6R)-4-(benzylamino)-2-ethyl-3-methyl-6-(3-nitropyridin-4-yl)tetrahydro-2H-pyran-3-ol in 61% yield. LCMS (m/z): 372.1 (MH⁺), Rₜ=0.64 min. ¹H NMR (400 MHz, CHLOROFORM-d) δ: 0.96 (t, J=7.34 Hz, 3H), 1.13 (s, 3H), 1.27-1.34 (m, 1H), 1.48 (ddd, J=14.16, 10.05, 7.19 Hz, 1H), 1.80 (ddd, J=14.09, 7.63, 1.76 Hz, 1H), 2.47-2.57 (m, 1H), 2.77 (dd, J=12.03, 4.11 Hz, 1H), 2.91 (br. s., 1H), 3.18 (dd, J=9.98, 1.76 Hz, 1H), 3.75 (d, J=12.91 Hz, 1H), 3.95 (d, J=12.91 Hz, 1H), 5.12 (dd, J=11.00, 1.91 Hz, 1H), 7.27 (dt, J=8.44, 4.44 Hz, 1H), 7.34 (d, J=4.40 Hz, 4H), 7.77 (d, J=4.99 Hz, 1H), 8.79 (d, J=4.99 Hz, 1H), 9.16 (s, 1H).

### Synthesis of tert-butyl ((2R,3S,4R,6R)-6-(3-aminopyridin-4-yl)-2-ethyl-3-hydroxy-3-methyltetrahydro-2H-pyran-4-yl)carbamate

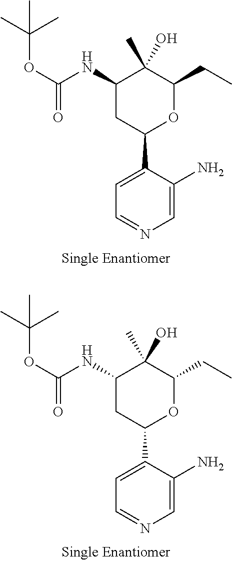

Method 12 was followed using (+/−)-(2R,3S,4R,6R)-4-(benzylamino)-2-ethyl-3-methyl-6-(3-nitropyridin-4-yl)tetrahydro-2H-pyran-3-ol (1.0 equiv.) and 10% Pearlman's catalyst (Pd hydroxide) (20 mol %) and Boc anhydride (1.0 equiv.) in MeOH/EtOAc (1:1, 0.15 M). Purification was completed via chiral HPLC (Ethanol/heptane=15/85, 20 mL/min, AD column) to yield in order of elution tert-butyl ((2R,3S,4R,6R)-6-(3-aminopyridin-4-yl)-2-ethyl-3-hydroxy-3-methyltetrahydro-2H-pyran-4-yl)carbamate (42% yield, 99% ee) and tert-butyl ((2S,3R,4S,6S)-6-(3-aminopyridin-4-yl)-2-ethyl-3-hydroxy-3-methyltetrahydro-2H-pyran-4-yl)carbamate (42% yield, 99% ee) LC/MS (m/z): 352.3 (MH⁺), Rₜ=0.54 min. ¹H NMR (CHLOROFORM-d) δ ppm 1.03 (t, J=7.43 Hz, 3H), 1.13 (s, 3H), 1.38-1.53 (m, 10H), 1.83 (br. s., 1H), 1.86-1.96 (m, 2H), 1.99 (dd, J=4.10, 2.82 Hz, 1H), 3.23 (d, J=8.71 Hz, 1H), 3.84 (br. s., 1H), 4.18-4.32 (m, 3H), 4.55 (dd, J=11.52, 2.30 Hz, 1H), 4.74 (br. s., 1H), 6.91 (d, J=4.86 Hz, 1H), 7.98 (d, J=4.86 Hz, 1H), 8.06 (s, 1H).

### Synthesis of (3,3-dimethoxybutan-2-ylidene)cyclopropane

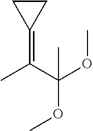

To a suspension of NaH (60% in mineral oil, 3.9 equiv.) in DME (0.5 M) was added (3-bromopropyl) triphenylphosphonium bromide portion wise at rt. The mixture was heated to 70° C. for 5 h. The reaction was cooled to rt and 3,3-dimethoxybutan-2-one was added. The reaction was stirred at 75° C. for 72 h. The mixture was cooled to rt, poured into ice water and extracted with pentane. The organic layer was dried over sodium sulfate, filtered and concentrated to give a red liquid. The crude product were purified by bulb to bulb distillation 90°-140°/10 torr to yield a clear liquid (75% y). ¹H NMR (400 MHz, CHLOROFORM-d) δ ppm 0.90-0.97 (m, 2H), 1.21 (td, J=7.43, 1.57 Hz, 2H), 1.40 (s, 3H), 1.82 (s, 3H), 3.13-3.19 (m, 6H).

### Synthesis of 3-cyclopropylidenebutan-2-one

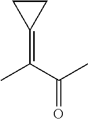

Water (1.0 equiv.) was added to a stirred suspension of silica gel (silica gel 60, 70-230 mesh, 10% water on silica) in DCM (0.6 M). After 5 min (water absorbed on to silica), (3,3-dimethoxybutan-2-ylidene)cyclopropane (1.0 equiv.) was added and the reaction was stirred at rt for 17 hrs. The mixture was filtered through a med frit glass funnel, eluting with DCM. The DCM was removed in vacuo to give 3-cyclopropylidenebutan-2-one in 74% yield. ¹H NMR (400 MHz, CHLOROFORM-d) δ ppm 1.24-1.32 (m, 2H), 1.48-1.57 (m, 2H), 1.95 (t, J=1.57 Hz, 3H), 2.37 (s, 3H).

### Synthesis of ((3-cyclopropylidenebut-1-en-2-yl)oxy)triethylsilane

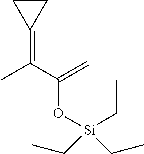

METHOD 6 was followed using 3-cyclopropylidenebutan-2-one (1.0 equiv.), LITHIUM BIS(TRIMETHYLSILYL)AMIDE(1.0 equiv.) and TRIETHYLCHLOROSILANE (1.05 equiv.) in THF (0.5 M) to give ((3-cyclopropylidenebut-1-en-2-yl)oxy)triethylsilane in 100% yield. ¹H NMR (400 MHz, CHLOROFORM-d) δ ppm 0.69-0.77 (m, 6H), 0.96-1.03 (m, 11H), 1.28-1.36 (m, 2H), 1.95 (t, J=1.57 Hz, 3H), 4.28 (s, 1H), 4.44 (s, 1H).

### Synthesis of (+/−)-(R)-4-(8-methyl-7-((triethylsilyl)oxy)-4-oxaspiro[2.5]oct-7-en-5-yl)-3-nitropyridine

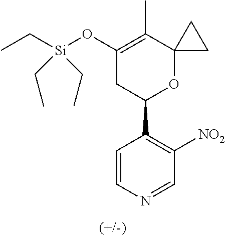 [SMILES: CC[Si](CC)(CC)OC1=C(C)C2(CC2)O[C@@H](c2ccncc2[N+](=O)[O-])C1]

METHOD 8 was followed using ((3-cyclopropylidenebut-1-en-2-yl)oxy)triethylsilane (1.0 equiv.), Eu(fod)₃ (0.05 equiv.) and 3-nitroisonicotinaldehyde (1.00 equiv.) in CHCl₃ (0.28 M) to yield (+/−)-(R)-4-(8-methyl-7-((triethylsilyl)oxy)-4-oxaspiro[2.5]oct-7-en-5-yl)-3-nitropyridine in 63% yield. LC/MS (m/z): 377.1 (MH⁺), Rₜ=1.31 min. ¹H NMR (CHLOROFORM-d) δ ppm 0.65-0.72 (m, 6H), 0.95-1.06 (m, 11H), 1.42 (dd, J=2.15, 1.37 Hz, 2H), 2.32-2.43 (m, 1H), 2.60-2.67 (m, 1H), 5.38 (dd, J=10.56, 3.52 Hz, 1H), 7.78 (d, J=5.09 Hz, 1H), 8.92 (d, J=4.70 Hz, 1H), 9.29 (s, 1H).

### Synthesis of (+/−)-(5R,8R)-8-hydroxy-8-methyl-5-(3-nitropyridin-4-yl)-4-oxaspiro[2.5]octan-7-one

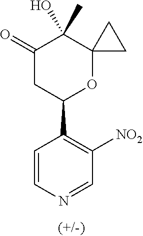 [SMILES: C[C@]1(O)C(=O)C[C@H](c2ccncc2[N+](=O)[O-])OC12CC2]

METHOD 10 was followed using (+/−)-(R)-4-(8-methyl-7-((triethylsilyl)oxy)-4-oxaspiro[2.5]oct-7-en-5-yl)-3-nitropyridine (1.0 equiv.) and 3,3-dimethyldioxirane as a solution in acetone (0.1M solution, 1.00 equiv.) in DCM (0.2 M) to give (+/−)-(5R,8R)-8-hydroxy-8-methyl-5-(3-nitropyridin-4-yl)-4-oxaspiro[2.5]octan-7-one in 45% yield. LC/MS (m/z): 279.1 (MH⁺), Rₜ=0.60 min. ¹H NMR (CHLOROFORM-d) δ ppm 0.63 (ddd, J=3.52, 6.75, 10.08 Hz, 1H), 0.87-0.99 (m, 3H), 1.68 (s, 3H), 2.86 (dd, J=11.54, 14.28 Hz, 1H), 3.13 (dd, J=3.13, 14.09 Hz, 1H), 3.75 (s, 1H), 5.40 (dd, J=2.74, 11.35 Hz, 1H), 7.85 (d, J=5.09 Hz, 1H), 8.89 (d, J=5.09 Hz, 1H), 9.21 (s, 1H).

### Synthesis of (+/−)-(5R,8S)-7-(benzylamino)-8-methyl-5-(3-nitropyridin-4-yl)-4-oxaspiro[2.5]octan-8-ol

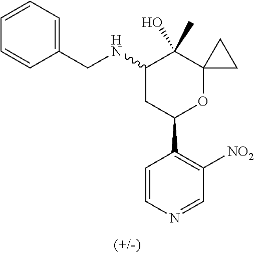 [SMILES: C[C@]1(O)C(NCc2ccccc2)C[C@H](c2ccncc2[N+](=O)[O-])OC12CC2]

(+/−)-(5R,8R)-8-hydroxy-8-methyl-5-(3-nitropyridin-4-yl)-4-oxaspiro[2.5]octan-7-one (1 equiv.) was dissolved in MeOH (0.3 M) and benzylamine was added at rt. The solution was stirred for 5 hrs at rt and then cooled to −78° C. and 2M LiBH₄ (1.1 equiv.) was added dropwise. The mixture was stirred allowing warming to rt overnight. The mixture was diluted with EtOAc and washed with sat. sodium bicarbonate, brine, dried over sodium sulfate, filtered and concentrated. The crude residue was purified by ISCO using an 80 g RediSep column eluting with 0-100% (10% MeOH in DCM) in DCM to yield (+/−)-(5R,8S)-7-(benzylamino)-8-methyl-5-(3-nitropyridin-4-yl)-4-oxaspiro[2.5]octan-8-ol in 72% yield. The two diastereomers were not separated. Their ratio was 74% and 26% by 10 min UPLC. LC/MS (m/z): 370.1 (MH⁺), Rₜ=0.58 min.

### Synthesis of tert-butyl ((5R,7S,8S)-5-(3-aminopyridin-4-yl)-8-hydroxy-8-methyl-4-oxaspiro[2.5]octan-7-yl)carbamate, tert-butyl ((5S,7S,8R)-5-(3-aminopyridin-4-yl)-8-hydroxy-8-methyl-4-oxaspiro[2.5]octan-7-yl)carbamate, tert-butyl ((5S,7R,8R)-5-(3-aminopyridin-4-yl)-8-hydroxy-8-methyl-4-oxaspiro[2.5]octan-7-yl)carbamate and tert-butyl ((5R,7R,8S)-5-(3-aminopyridin-4-yl)-8-hydroxy-8-methyl-4-oxaspiro[2.5]octan-7-yl)carbamate

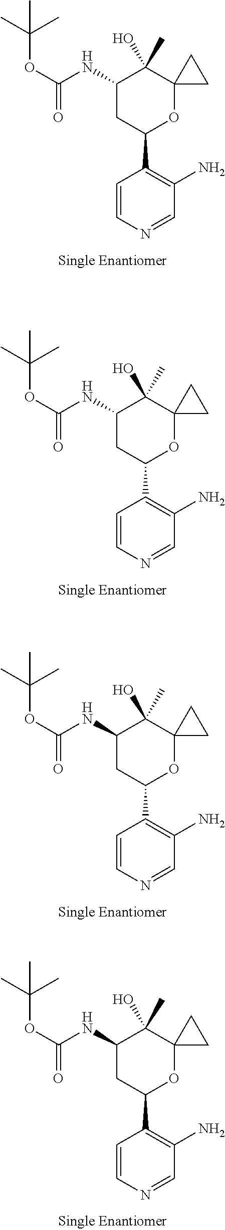 [SMILES: CC(C)(C)OC(=O)N[C@H]1C[C@H](c2ccncc2N)OC2(CC2)[C@@]1(C)O ; CC(C)(C)OC(=O)N[C@H]1C[C@@H](c2ccncc2N)OC2(CC2)[C@]1(C)O ; CC(C)(C)OC(=O)N[C@@H]1C[C@@H](c2ccncc2N)OC2(CC2)[C@]1(C)O ; CC(C)(C)OC(=O)N[C@@H]1C[C@H](c2ccncc2N)OC2(CC2)[C@@]1(C)O]

(+/−)-(5R,8S)-7-(benzylamino)-8-methyl-5-(3-nitropyridin-4-yl)-4-oxaspiro[2.5]octan-8-ol (1.0 equiv.) was dissolved in MeOH (0.2 M) and degassed with vacuum to Argon 3 times. 10% Pearlman's catalyst (Pd hydroxide) (20 mol %) was added and the resulting mixture was evacuated and backfilled with hydrogen gas (three times) and the mixture was then stirred at room temperature under the H₂ balloon for 18 h. The H₂ was removed by vacuum and the reaction purged with N₂. Boc₂O (2.0 equiv.) was added and the mixture stirred at rt for 2 h. The mixture was filtered through celite eluting with EtOAc and concentrated. The crude material was purified by ISCO using a 40 g RediSep column, dry loading, eluting with 0-10% (10% MeOH in DCM) in DCM to give two diastereomers in 71% yield. Purification was completed via chiral HPLC (Heptane/EtOH=90/10, 20 mL/min, AD column) to yield in order of elution tert-butyl ((5R,7S,8S)-5-(3-aminopyridin-4-yl)-8-hydroxy-8-methyl-4-oxaspiro[2.5]octan-7-yl)carbamate (19% y, 99% ee), tert-butyl ((5S,7S,8R)-5-(3-aminopyridin-4-yl)-8-hydroxy-8-methyl-4-oxaspiro[2.5]octan-7-yl)carbamate (6% y, 99% ee), tert-butyl ((5S,7R,8R)-5-(3-aminopyridin-4-yl)-8-hydroxy-8-methyl-4-oxaspiro[2.5]octan-7-yl)carbamate (23% y, 99% ee) and tert-butyl ((5R,7R,8S)-5-(3-aminopyridin-4-yl)-8-hydroxy-8-methyl-4-oxaspiro[2.5]octan-7-yl)carbamate (7% yield, 99% ee) LC/MS (m/z): 350.1 (MH⁺), Rₜ=0.52 min. ¹H NMR shows that Peaks 1 and 3 were one set of enantiomers and peaks 2 and 4 the other. Peak 1-¹H NMR (CHLOROFORM-d) δ ppm 0.62 (d, J=5.48 Hz, 1H), 0.76-0.82 (m, 1H), 0.90 (m, 1H), 0.98-1.09 (m, 1H), 1.27 (br. s., 3H), 1.45-1.49 (m, 9H), 2.18 (d, J=7.04 Hz, 1H), 2.45 (br. s., 1H), 3.99 (br. s., 1H), 4.17 (br. s., 2H), 4.76 (dd, J=10.56, 2.35 Hz, 1H), 5.30 (br. s., 1H), 7.01 (d, J=4.70 Hz, 1H) 7.96 (d, J=5.09 Hz, 1H), 8.00 (s, 1H). Peak 2-¹H NMR (CHLOROFORM-d) δ ppm 0.60-0.71 (m, 1H), 0.76 (dd, J=10.96, 5.09 Hz, 1H), 0.90 (dd, J=9.98, 6.06 Hz, 1H), 1.12 (dd, J=9.78, 5.09 Hz, 1H), 1.39 (s, 3H), 1.42-1.49 (m, 9H), 1.96-2.05 (m, 2H), 3.92-4.05 (m, 2H), 4.14-4.22 (m, 2H), 4.63 (dd, J=10.56, 3.52 Hz, 1H), 4.75 (d, J=6.26 Hz, 1H), 6.90 (d, J=4.70 Hz, 1H), 7.97 (d, J=5.09 Hz, 1H), 8.03 (s, 1H).

### Synthesis of (E)-triethyl(hexa-1,3-dien-2-yloxy)silane

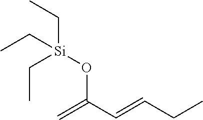

METHOD 7 was followed using (E)-hex-3-en-2-one (1.0 equiv.), TESOTf (1.2 equiv.) and Et₃N (1.4 equiv.) in THF (0.25 M) to give (E)-triethyl(hexa-1,3-dien-2-yloxy)silane in 100% yield.

Synthesis of (+/−)-4-((2R,6R)-6-ethyl-4-((triethylsilyl)oxy)-3,6-dihydro-2H-pyran-2-yl)-3-nitropyridine

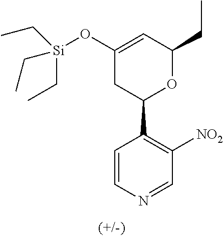 [SMILES: CC[C@@H]1C=C(O[Si](CC)(CC)CC)C[C@H](c2ccncc2[N+](=O)[O-])O1]

Method 8 was followed using (E)-triethyl(hexa-1,3-dien-2-yloxy)silane (1.0 equiv.), Eu(fod)₃ (0.05 equiv.) and 3-nitroisonicotinaldehyde (1.2 equiv.) in CHCl₃ (0.25 M) to yield (+/−)-4-((2R,6R)-6-ethyl-4-((triethylsilyl)oxy)-3,6-dihydro-2H-pyran-2-yl)-3-nitropyridine in 68% yield. LC/MS (m/z): 365.0 (MH⁺), Rₜ=1.30 min. ¹H NMR (CHLOROFORM-d) δ ppm 0.73 (q, J=7.93 Hz, 6H), 0.97-1.08 (m, 12H), 1.59-1.78 (m, 2H), 2.25-2.38 (m, 1H), 2.54-2.66 (m, 1H), 4.33 (br. s., 1H), 4.91 (s, 1H), 5.43 (dd, J=10.57, 2.64 Hz, 1H), 8.04 (d, J=4.29 Hz, 1H), 9.47 (br. s., 1H), 9.73-10.01 (m, 1H).

### Synthesis of (+/−)-(2R,3R,6R)-2-ethyl-3-hydroxy-6-(3-nitropyridin-4-yl)dihydro-2H-pyran-4(3H)-one

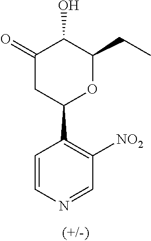 [SMILES: CC[C@H]1O[C@@H](c2ccncc2[N+](=O)[O-])CC(=O)[C@@H]1O]

Method 9 was followed using (+/−)-4-((2R,6R)-6-ethyl-4-((triethylsilyl)oxy)-3,6-dihydro-2H-pyran-2-yl)-3-nitropyridine (1.0 equiv.), acetone (10.0 equiv.), NaHCO₃ (5.0 equiv.) and oxone (1.1 equv.) in EtOAc:water 1:1(0.13 M) to give (+/−)-(2R,3R,6R)-2-ethyl-3-hydroxy-6-(3-nitropyridin-4-yl)dihydro-2H-pyran-4(3H)-one in 49% yield. LC/MS (m/z): 267.0 (MH⁺), Rₜ=0.55 min. ¹H NMR (CHLOROFORM-d) δ ppm 1.07 (t, J=7.51 Hz, 3H), 1.78 (dquin, J=14.72, 7.36, 7.36, 7.36, 7.36 Hz, 1H), 2.02-2.14 (m, 1H), 2.56-2.65 (m, 1H), 3.15 (dd, J=13.82, 2.40 Hz, 1H), 3.41-3.49 (m, 1H), 4.04 (d, J=9.61 Hz, 1H), 5.35 (dd, J=11.26, 2.25 Hz, 1H), 7.86 (d, J=5.11 Hz, 1H), 8.91 (d, J=5.11 Hz, 1H), 9.24 (s, 1H).

### Synthesis of (+/−)-(2R,3S,4R,6R)-4-(benzylamino)-2-ethyl-6-(3-nitropyridin-4-yl)tetrahydro-2H-pyran-3-ol

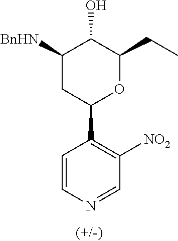

Method 11 was followed using (+/−)-(2R,3R,6R)-2-ethyl-3-methyl-6-(3-nitropyridin-4-yl)dihydro-2H-pyran-4(3H)-one (1.0 equiv.), benzylamine (3.0 equiv.) and 2M LiBH₄ (1.2 equiv.) in MeOH (0.28 M) to give (+/−)-(2R,3S,4R,6R)-4-(benzylamino)-2-ethyl-6-(3-nitropyridin-4-yl)tetrahydro-2H-pyran-3-ol in 21% yield.

LC/MS (m/z): 358.1 (MH⁺), Rₜ=0.59 min. ¹H NMR (CHLOROFORM-d) δ ppm 1.00 (t, J=7.36 Hz, 3H), 1.21-1.29 (m, 1H), 1.59 (tt, J=14.79, 7.73 Hz, 1H), 1.96 (dqd, J=14.53, 7.37, 7.37, 7.37, 2.40 Hz, 1H), 2.56-2.64 (m, 1H), 2.76-2.87 (m, 1H), 3.13 (t, J=9.31 Hz, 1H), 3.26-3.36 (m, 1H), 3.75 (d, J=12.92 Hz, 1H), 3.94 (d, J=12.92 Hz, 1H), 5.11 (d, J=9.61 Hz, 1H), 7.28-7.39 (m, 5H), 7.76-7.80 (m, 1H), 8.79-8.83 (m, 1H), 9.17-9.21 (m, 1H).

### Synthesis of tert-butyl ((2R,3S,4R,6R)-6-(3-aminopyridin-4-yl)-2-ethyl-3-hydroxytetrahydro-2H-pyran-4-yl)carbamate and tert-butyl ((2S,3R,4S,6S)-6-(3-aminopyridin-4-yl)-2-ethyl-3-hydroxytetrahydro-2H-pyran-4-yl)carbamate

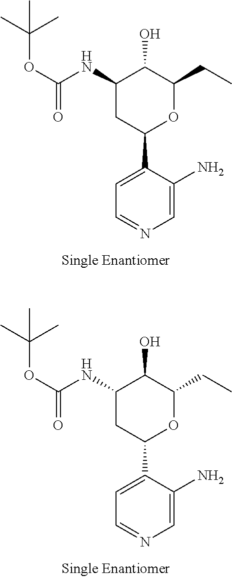

Method 12 was followed using (+/−)-(2R,3S,4R,6R)-4-(benzylamino)-2-ethyl-6-(3-nitropyridin-4-yl)tetrahydro-2H-pyran-3-ol (1.0 equiv.) and 20% Pearlman's catalyst (Pd hydroxide) (20 mol %) and Boc anhydride (1.1 equiv.) in MeOH/EtOAc (4:1, 0.10 M). Purification was completed via chiral HPLC (Heptane/IPA=85/15, 20 mL/min, AD column) to yield in order of elution tert-butyl ((2R,3S,4R,6R)-6-(3-aminopyridin-4-yl)-2-ethyl-3-hydroxytetrahydro-2H-pyran-4-yl)carbamate (33% yield, 99% ee) and tert-butyl ((2S,3R,4S,6S)-6-(3-aminopyridin-4-yl)-2-ethyl-3-hydroxytetrahydro-2H-pyran-4-yl)carbamate (34% yield, 99% ee) LC/MS (m/z): 338.2 (MH⁺), Rₜ=0.48 min. ¹H NMR (CHLOROFORM-d) δ ppm 1.01 (t, J=7.33 Hz, 3H), 1.43-1.48 (m, 9H), 1.90 (d, J=12.38 Hz, 1H), 1.97-2.08 (m, 1H), 2.14 (br. s., 1H), 3.23 (d, J=9.10 Hz, 1H), 3.30 (dd, J=8.08, 2.53 Hz, 1H), 3.71-3.81 (m, 1H), 4.22 (br. s., 2H), 4.51 (dd, J=11.50, 1.89 Hz, 1H), 4.62-4.72 (m, 1H), 6.92 (d, J=4.80 Hz, 1H), 7.98 (d, J=4.80 Hz, 1H), 8.06 (s, 1H).

### Synthesis of (E)-triethyl((5-methylhexa-1,3-dien-2-yl)oxy)silane

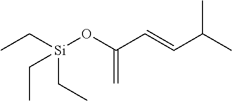

METHOD 7 was followed using 5-methyl-3-hexen-2-one, TESOTf (1.1 equiv.) and Et₃N (2.0 equiv.) in Et₂O (0.25 M) to give (E)-triethyl((5-methylhexa-1,3-dien-2-yl)oxy)silane in 100% yield. ¹H NMR (CHLOROFORM-d) δ ppm 0.72 (t, J=6.85 Hz, 6H), 0.89-1.11 (m, 15H), 4.22 (br. s., 2H), 5.78-5.88 (m, 1H), 5.94-6.07 (m, 1H).

### Synthesis of (+/−)-4-((2R,6R)-6-isopropyl-4-((triethylsilyl)oxy)-3,6-dihydro-2H-pyran-2-yl)-3-nitropyridine

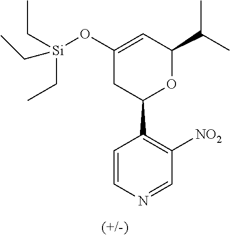 [SMILES: CC[Si](CC)(CC)OC1=C[C@@H](C(C)C)O[C@@H](c2ccncc2[N+](=O)[O-])C1]

Method 8 was followed using (E)-triethyl((5-methylhexa-1,3-dien-2-yl)oxy)silane (1.0 equiv.), Eu(fod)₃ (0.05 equiv.) and 3-nitroisonicotinaldehyde (1.4 equiv.) in CHCl₃ (0.20 M) to yield (+/−)-4-((2R,6R)-6-isopropyl-4-((triethylsilyl)oxy)-3,6-dihydro-2H-pyran-2-yl)-3-nitropyridine in 63% yield. LC/MS (m/z): 379.1 (MH⁺), Rₜ=1.40 min. ¹H NMR (CHLOROFORM-d) δ ppm 0.69-0.76 (m, 6H), 0.99-1.05 (m, 15H), 1.84-1.94 (m, 1H), 2.28-2.37 (m, 1H), 2.61 (dt, J=16.04, 2.74 Hz, 1H), 4.20-4.25 (m, 1H), 4.92 (t, J=1.76 Hz, 1H), 5.44 (dd, J=10.56, 3.13 Hz, 1H), 8.06 (d, J=4.70 Hz, 1H), 9.59 (br. s., 1H), 9.98 (br. s., 1H).

### Synthesis of (+/−)-(2S,6R)-2-isopropyl-6-(3-nitropyridin-4-yl) dihydro-2H-pyran-4(3H)-one and (+/−)-(2R,3R,6R)-3-hydroxy-2-isopropyl-6-(3-nitropyridin-4-yl) dihydro-2H-pyran-4(3H)-one

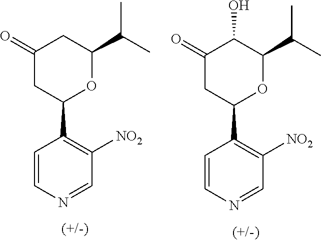

METHOD 10 was followed using (+/−)-4-((2R,6R)-6-isopropyl-4-((triethylsilyl)oxy)-3,6-dihydro-2H-pyran-2-yl)-3-nitropyridine (1.0 equiv.) and 3,3-dimethyldioxirane as a solution in acetone (0.1M solution, 1.1 equiv.) in DCM (0.15 M) to give (+/−)-(2S,6R)-2-isopropyl-6-(3-nitropyridin-4-yl)dihydro-2H-pyran-4(3H)-one in 15% yield, LC/MS (m/z): 265.0 (MH⁺), Rₜ=0.77 min; and (+/−)-(2R,3R,6R)-3-hydroxy-2-isopropyl-6-(3-nitropyridin-4-yl)dihydro-2H-pyran-4(3H)-one in 50% yield, LC/MS (m/z): 281.0 (MH⁺), Rₜ=0.65 min, ¹H NMR (CHLOROFORM-d) δ ppm 1.10 (dd, J=13.30, 7.04 Hz, 6H), 2.25 (dtd, J=14.09, 7.04, 7.04, 1.96 Hz, 1H), 2.59 (ddd, J=13.40, 11.64, 1.17 Hz, 1H), 3.15 (dd, J=13.69, 2.35 Hz, 1H), 3.40 (dd, J=10.17, 2.35 Hz, 1H), 3.60 (d, J=3.52 Hz, 1H), 4.18 (d, J=9.78 Hz, 1H), 5.32 (dd, J=11.54, 2.15 Hz, 1H), 7.82 (d, J=5.09 Hz, 1H), 8.91 (d, J=5.09 Hz, 1H), 9.24 (s, 1H).

### Synthesis of (+/−)-(2R,3S,4R,6R)-4-(benzylamino)-2-isopropyl-6-(3-nitropyridin-4-yl)tetrahydro-2H-pyran-3-ol

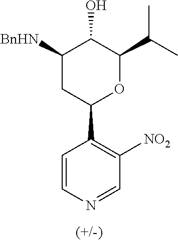

Method 11 was followed using (+/−)-(2R,3R,6R)-3-hydroxy-2-isopropyl-6-(3-nitropyridin-4-yl)dihydro-2H-pyran-4(3H)-one (1.0 equiv.), benzylamine (3.0 equiv.) and 2M LiBH₄ (1.1 equiv.) in MeOH (0.27 M) to give (+/−)-(2R,3S,4R,6R)-4-(benzylamino)-2-isopropyl-6-(3-nitropyridin-4-yl)tetrahydro-2H-pyran-3-ol in 25% yield. LC/MS (m/z): 372.0 (MH⁺), Rₜ=0.63 min. ¹H NMR (CHLOROFORM-d) δ ppm 1.01 (d, J=7.04 Hz, 5H), 1.20 (t, J=10.96 Hz, 1H), 2.19 (dt, J=14.18, 6.80 Hz, 1H), 2.59 (ddd, J=12.72, 4.11, 1.96 Hz, 1H), 2.78-2.86 (m, 1H), 3.27 (d, J=0.78 Hz, 1H), 3.75 (d, J=12.91 Hz, 1H), 3.94 (d, J=13.30 Hz, 1H), 5.09 (dd, J=10.96, 1.57 Hz, 1H), 7.24-7.39 (m, 5H), 7.75 (d, J=5.09 Hz, 1H), 8.81 (d, J=5.48 Hz, 1H), 9.19 (s, 1H).

### Synthesis of tert-butyl ((2R,3S,4R,6R)-6-(3-aminopyridin-4-yl)-3-hydroxy-2-isopropyltetrahydro-2H-pyran-4-yl)carbamate and tert-butyl ((2S,3R,4S,6S)-6-(3-aminopyridin-4-yl)-3-hydroxy-2-isopropyltetrahydro-2H-pyran-4-yl)carbamate

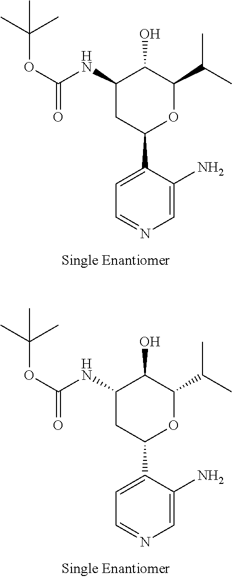

Method 12 was followed using (+/−)-(2R,3S,4R,6R)-4-(benzylamino)-2-isopropyl-6-(3-nitropyridin-4-yl)tetrahydro-2H-pyran-3-ol (1.0 equiv.) and 20% Pearlman's catalyst (Pd hydroxide) (20 mol %) and Boc anhydride (1.05 equiv.) in MeOH (0.10 M). Purification was completed via chiral HPLC (Heptane/IPA/=85/15, mL/min, AD column) to yield in order of elution tert-butyl ((2R,3S,4R,6R)-6-(3-aminopyridin-4-yl)-3-hydroxy-2-isopropyltetrahydro-2H-pyran-4-yl)carbamate (27% yield, 99% ee) and tert-butyl ((2S,3R,4S,6S)-6-(3-aminopyridin-4-yl)-3-hydroxy-2-isopropyltetrahydro-2H-pyran-4-yl)carbamate (25% yield, 99% ee). LC/MS (m/z): 338.2 (MH⁺), Rₜ=0.48 min. ¹H NMR (CHLOROFORM-d) δ ppm 0.95 (d, J=7.04 Hz, 3H), 1.05 (d, J=7.04 Hz, 3H), 1.46 (s, 10H), 1.88 (q, J=1.00 Hz, 1H), 2.12 (ddd, J=12.91, 4.70, 2.35 Hz, 1H), 2.29 (quind, J=7.04, 7.04, 7.04, 7.04, 1.96 Hz, 1H), 3.25 (dd, J=9.39, 1.96 Hz, 1H), 3.33-3.40 (m, 1H), 3.71-3.83 (m, 1H), 4.23 (s, 2H), 4.49 (dd, J=11.54, 2.15 Hz, 1H), 4.67 (br. s., 1H), 6.91 (d, J=5.09 Hz, 1H), 7.98 (d, J=4.70 Hz, 1H), 8.05 (s, 1H).

### Synthesis of (+/−)-(2S,4S,6R)—N-benzyl-2-isopropyl-6-(3-nitropyridin-4-yl)tetrahydro-2H-pyran-4-amine

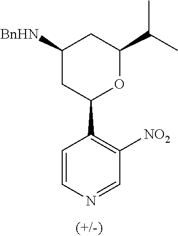

Method 11 was followed using (+/−)-(2S,6R)-2-isopropyl-6-(3-nitropyridin-4-yl)dihydro-2H-pyran-4(3H)-one (1.0 equiv.), benzylamine (2.0 equiv.) and 2M LiBH₄ (1.1 equiv.) in MeOH (0.28 M) to give (+/−)-(2S,4S,6R)—N-benzyl-2-isopropyl-6-(3-nitropyridin-4-yl)tetrahydro-2H-pyran-4-aminein 100% yield. The crude was used in next step without further purification. LC/MS (m/z): 356.0 (MH⁺), Rₜ=0.70 min.

### Synthesis of tert-butyl ((2R,4S,6S)-2-(3-aminopyridin-4-yl)-6-isopropyltetrahydro-2H-pyran-4-yl)carbamate and tert-butyl ((2S,4R,6R)-2-(3-aminopyridin-4-yl)-6-isopropyltetrahydro-2H-pyran-4-yl)carbamate

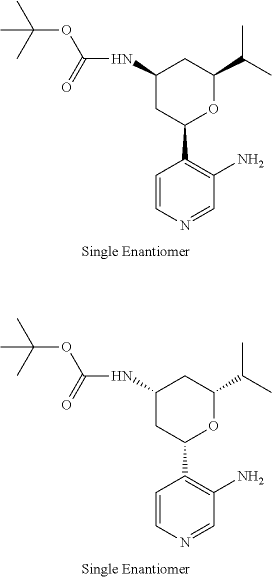

Method 12 was followed using (+/−)-(2S,4S,6R)—N-benzyl-2-isopropyl-6-(3-nitropyridin-4-yl)tetrahydro-2H-pyran-4-amine (1.0 equiv.) and 20% Pearlman's catalyst (Pd hydroxide) (20 mol %) and Boc anhydride (1.1 equiv.) in MeOH (0.15 M). Purification was completed via SFC (IPA+0.1% DEA=25%, 15 mL/min, IC column) to yield in order of elution tert-butyl ((2R,4S,6S)-2-(3-aminopyridin-4-yl)-6-isopropyltetrahydro-2H-pyran-4-yl)carbamate (23% yield, 99% ee) and tert-butyl ((2S,4R,6R)-2-(3-aminopyridin-4-yl)-6-isopropyltetrahydro-2H-pyran-4-yl)carbamate (22% yield, 99% ee). LC/MS (m/z): 336.1 (MH⁺), Rₜ=0.71 min. ¹H NMR (CHLOROFORM-d) δ ppm 0.96 (t, J=6.99 Hz, 6H), 1.11-1.23 (m, 1H), 1.39-1.52 (m, 9H), 1.63 (d, J=12.21 Hz, 1H), 1.79 (dd, J=12.97, 6.61 Hz, 1H), 2.04 (dt, J=10.24, 2.00 Hz, 1H), 2.15 (d, J=12.46 Hz, 1H), 3.26-3.36 (m, 1H), 3.77-3.93 (m, 1H), 4.25 (s, 2H), 4.40-4.47 (m, 1H), 4.49-4.58 (m, 1H), 6.93 (d, J=4.83 Hz, 1H), 7.97 (d, J=4.83 Hz, 1H), 8.04 (s, 1H).

### Synthesis of triethyl((4-methylpenta-1,3-dien-2-yl)oxy)silane

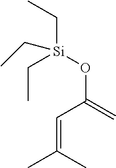

METHOD 7 was followed using 4-methylpent-3-en-2-one, TESOTf (1.0 equiv.) and Et₃N (1.4 equiv.) in DCM (0.24 M) to give triethyl((4-methylpenta-1,3-dien-2-yl)oxy)silane in 99% yield. ¹H NMR (CHLOROFORM-d) δ ppm 0.69-0.76 (m, 6H), 0.96-1.01 (m, 9H), 1.76 (s, 3H), 1.91 (s, 3H), 4.14 (s, 1H), 4.27 (s, 1H), 5.58 (s, 1H).

### Synthesis of (+/−)-1-hydroxy-5-methyl-1-(3-nitropyridin-4-yl)hex-4-en-3-one

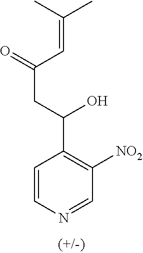 [SMILES: CC(C)=CC(=O)CC(O)c1ccncc1[N+](=O)[O-]]

To a solution of triethyl((4-methylpenta-1,3-dien-2-yl)oxy)silane (1 equiv.) in CHCl₃ (0.48 M) was added 3-nitroisonicotinaldehyde (2.4 equiv.) and Eu(fod)₃ (0.05 equiv.). The solution was submerged in a 60° C. oil bath and left stirring for 90 min. the reaction was removed from the oil bath and the volatiles were removed in vacuo and the material was purified by ISCO using a 330 g column, eluting with 0-40% EtOAc/n-heptanes to yield (+/−)-1-hydroxy-5-methyl-1-(3-nitropyridin-4-yl)hex-4-en-3-one in 22% yield. LC/MS (m/z): 251.1 (MH⁺), Rₜ=0.61 min. ¹H NMR (CHLOROFORM-d) δ ppm 1.94 (s, 3H), 2.22 (s, 3H), 2.63 (dd, J=17.61, 9.10 Hz, 1H), 3.09 (dd, J=17.46, 2.20 Hz, 1H), 4.33 (d, J=2.93 Hz, 1H), 5.78 (dt, J=9.17, 2.31 Hz, 1H), 6.05 (s, 1H), 7.91 (d, J=5.28 Hz, 1H), 8.84 (d, J=4.99 Hz, 1H), 9.21 (s, 1H).

### Synthesis of (+/−)-2,2-dimethyl-6-(3-nitropyridin-4-yl)dihydro-2H-pyran-4(3H)-one

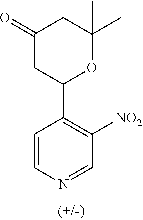 [SMILES: CC1(C)CC(=O)CC(c2ccncc2[N+](=O)[O-])O1]

To a solution of 1-hydroxy-5-methyl-1-(3-nitropyridin-4-yl)hex-4-en-3-one (1 equiv.) in CH₂Cl₂ (0.25 M) was added Amberlyst-15 acidic resin, 20-50 mesh, 4.7 equiv H+/gram (19.8 equiv.). After stirring at rt for 4 days, the resin was filtered eluting with CH₂Cl₂ and the organic was washed with Na₂CO_{3(sat.)} and NaCl_{(sat.)}, dried over MgSO₄ filtered and concentrated to yield 1.5 grams crude. In case the product was sticking to the acidic resin, the resin was rinsed with 1% Et₃N/CH₂Cl₂ and the volatiles were removed in vacuo to yield additional product. The combined crude products were purified by ISCO SiO₂ chromatography (80 gram column, 0-100% EtOAc/n-heptanes, developed tlc in 50% EtOAc/n-heptanes) to yield (+/−)-2,2-dimethyl-6-(3-nitropyridin-4-yl)dihydro-2H-pyran-4(3H)-one in 65% yield (UPLC 91% by UV). LC/MS (m/z): 251.1 (MH⁺), Rₜ=0.67 min. ¹H NMR (CHLOROFORM-d) δ ppm 1.32 (s, 3H), 1.47 (s, 3H), 2.34 (dd, J=14.23, 11.30 Hz, 1H), 2.42-2.59 (m, 2H), 2.83-2.92 (m, 1H), 5.55 (dd, J=11.30, 2.79 Hz, 1H), 7.86 (d, J=5.28 Hz, 1H), 8.87 (d, J=4.99 Hz, 1H), 9.18 (s, 1H).

### Synthesis of (+/−)-(4S,6R)—N-benzyl-2,2-dimethyl-6-(3-nitropyridin-4-yl)tetrahydro-2H-pyran-4-amine

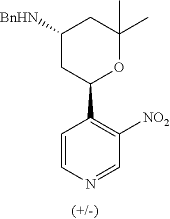

Method 11 was followed using (+/−)-2,2-dimethyl-6-(3-nitropyridin-4-yl)dihydro-2H-pyran-4(3H)-one (1.0 equiv.), benzylamine (3.0 equiv.) and 2M LiBH4 (1.0 equiv.) in MeOH (0.2 M) to give (+/−)-(4S,6R)—N-benzyl-2,2-dimethyl-6-(3-nitropyridin-4-yl)tetrahydro-2H-pyran-4-amine

in 100% yield. The crude was used in next step without further purification. LC/MS (m/z): 342.1 (MH⁺), Rₜ=0.60 min.

### Synthesis of tert-butyl ((4S,6R)-6-(3-aminopyridin-4-yl)-2,2-dimethyltetrahydro-2H-pyran-4-yl)carbamate and tert-butyl a4R,6S)-6-(3-aminopyridin-4-yl)-2,2-dimethyltetrahydro-2H-pyran-4-yl)carbamate

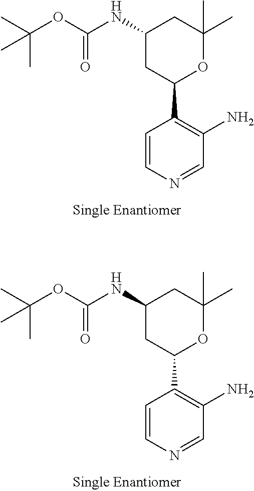

Method 12 was followed using (+/−)-(4S,6R)—N-benzyl-2,2-dimethyl-6-(3-nitropyridin-4-yl)tetrahydro-2H-pyran-4-amine (1.0 equiv.), 20% Pearlman's catalyst (Pd hydroxide) (20 mol %) and Boc anhydride (1.05 equiv.) in MeOH (0.2 M). Purification was completed via chiral HPLC (Heptane/EtOH/=90/10, 20 mL/min, AD column) to yield in order of elution tert-butyl ((4S,6R)-6-(3-aminopyridin-4-yl)-2,2-dimethyltetrahydro-2H-pyran-4-yl)carbamate (20% yield, 99% ee) and tert-butyl ((4R,6S)-6-(3-aminopyridin-4-yl)-2,2-dimethyltetrahydro-2H-pyran-4-yl)carbamate (18% yield, 98% ee). LC/MS (m/z): 322.1 (MH⁺), Rₜ=0.62 min. ¹H NMR (CHLOROFORM-d) δ ppm 1.22 (s, 3H), 1.40-1.51 (m, 12H), 1.74-1.88 (m, 3H), 2.23-2.34 (m, 1H), 4.06 (br. s., 1H), 4.33, (br. s., 2H), 4.68 (br. s., 1H), 4.87 (dd, J=9.68, 2.93 Hz, 1H), 7.02 (d, J=4.70 Hz, 1H), 7.99 (d, J=4.70 Hz, 1H), 8.03 (s, 1H).

### Synthesis of (E)-ethyl 4-acetylhex-4-enoate

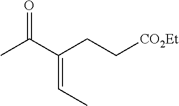

To a solution of (E)-pent-3-en-2-one (1.0 equiv.) in DMI (1,3-dimethyl-2-imidazolidinone) (0.58 M) was added ethyl acrylate (1.3 equiv.) and DBU (0.2 equiv.) in a steel bomb. The reaction was heated at 165° C. for 16 h and 185° C. for another 24 h. The reaction was cooled to room temperature and worked up by the addition of water and ether. The aqueous phase was extracted twice with ether. The organic layer was washed with Brine and dried with sodium sulfate, filtered and concentrated. The crude material was purified ISCO Combi-flash Rf system with a Redisep column eluting with 0-40% Ether/pentane to yield (E)-ethyl 4-acetylhex-4-enoate in 44% yield.). LC/MS (m/z): 185.1 (MH⁺), Rₜ=0.64 min. ¹H NMR (CHLOROFORM-d) δ ppm 1.18-1.23 (m, 3H), 1.92 (d, J=7.04 Hz, 3H), 2.27-2.32 (m, 3H), 2.35 (t, J=7.83 Hz, 2H), 2.58-2.65 (m, 2H), 4.11 (m, J=7.04, 7.04, 7.04 Hz, 2H), 6.80 (q, J=7.04 Hz, 1H).

### Synthesis of (E)-ethyl 4-(1-((triethylsilyl)oxy)vinyl)hex-4-enoate

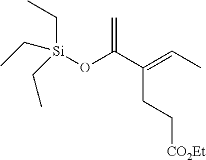 [SMILES: C=C(O[Si](CC)(CC)CC)/C(=C/C)CCC(=O)OCC]

METHOD 7 was followed using (E)-ethyl 4-acetylhex-4-enoate (1.0 equiv.), TESOTf (1.0 equiv.) and Et₃N (2.0 equiv.) in THF (0.17 M) to give (E)-ethyl 4-(1-((triethylsilyl)oxy)vinyl)hex-4-enoate in 100% yield.

### Synthesis of (+/−)-ethyl 3-((2R,6R)-2-methyl-6-(3-nitropyridin-4-O-4-((triethylsilyl)oxy)-5,6-dihydro-2H-pyran-3-yl)propanoate

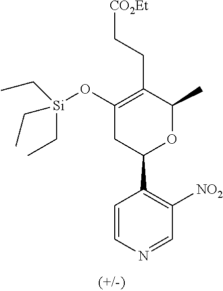 [SMILES: CCOC(=O)CCC1=C(O[Si](CC)(CC)CC)C[C@H](c2ccncc2[N+](=O)[O-])O[C@@H]1C]

Method 8 was followed using (E)-ethyl 4-(1-((triethylsilyl)oxy)vinyl)hex-4-enoate (1.0 equiv.), Eu(fod)₃ (0.05 equiv.) and 3-nitroisonicotinaldehyde (1.2 equiv.) in CHCl₃ (0.25 M) to yield (+/−)-ethyl 3-((2R,6R)-2-methyl-6-(3-nitropyridin-4-yl)-4-((triethylsilyl)oxy)-5,6-dihydro-2H-pyran-3-yl)propanoate in 33% yield. LC/MS (m/z): 451.3 (MH⁺), Rₜ=1.37 min. ¹H NMR (CHLOROFORM-d) δ ppm 0.63-0.72 (m, 6H), 1.01 (s, 9H), 1.27 (t, J=7.04 Hz, 3H), 1.32-1.38 (m, 3H), 2.18-2.31 (m, 2H), 2.32-2.42 (m, 1H), 2.43-2.55 (m, 2H), 2.56-2.66 (m, 1H), 4.15 (q, J=7.04 Hz, 2H), 4.37-4.45 (m, 1H), 5.17 (dd, J=10.42, 2.79 Hz, 1H), 7.84 (d, J=5.28 Hz, 1H), 8.88 (d, J=4.99 Hz, 1H), 9.21 (s, 1H).

### Synthesis of ethyl 3-((2R,3R,6R)-3-hydroxy-2-methyl-6-(3-nitropyridin-4-yl)-4-oxotetrahydro-2H-pyran-3-yl)propanoate

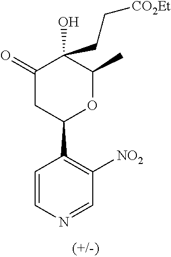 [SMILES: CCOC(=O)CC[C@]1(O)C(=O)C[C@H](c2ccncc2[N+](=O)[O-])O[C@@H]1C]

Method 9 was followed using (+/−)-ethyl 3-((2R,6R)-2-methyl-6-(3-nitropyridin-4-yl)-4-((triethylsilyl)oxy)-5,6-dihydro-2H-pyran-3-yl)propanoate (1.0 equiv.), acetone (10.0 equiv.), NaHCO₃ (5.0 equiv.) and oxone (1.3 equv.) in EtOAc:water 1:1(0.15 M) to give (+/−)-ethyl 3-((2R,3R,6R)-3-hydroxy-2-methyl-6-(3-nitropyridin-4-yl)-4-oxotetrahydro-2H-pyran-3-yl)propanoate in 20% yield. LC/MS (m/z): 353.0 (MH⁺), Rₜ=0.70 min. ¹H NMR (CHLOROFORM-d) δ ppm 1.19-1.23 (m, 3H), 1.37-1.44 (m, 3H), 2.05-2.14 (m, 1H), 2.15-2.26 (m, 1H), 2.31-2.44 (m, 2H) 2.79-2.89 (m, 1H), 3.07 (dd, J=13.60, 2.66 Hz, 1H), 3.65 (q, J=6.41 Hz, 1H), 3.96 (s, 1H), 4.03-4.09 (m, 2H), 5.33 (dd, J=11.39, 2.51 Hz, 1H), 7.89 (d, J=5.03 Hz, 1H), 8.89 (d, J=5.03 Hz, 1H), 9.21 (s, 1H).

### Synthesis of (+/−)-(4aS,5R,7R,8aR)-1-benzyl-4a-hydroxy-5-methyl-7-(3-nitropyridin-4-yl)hexahydro-1H-pyrano[4,3-b]pyridin-2(7H)-one

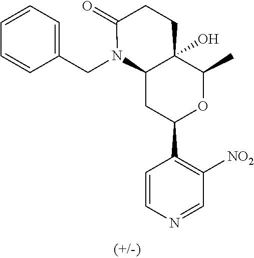 [SMILES: C[C@H]1O[C@@H](c2ccncc2[N+](=O)[O-])C[C@H]2N(Cc3ccccc3)C(=O)CC[C@@]12O]

To a round-bottom flask containing (+/−)-ethyl 3-((2R,3R,6R)-3-hydroxy-2-methyl-6-(3-nitropyridin-4-yl)-4-oxotetrahydro-2H-pyran-3-yl)propanoate (1.0 equiv.) in 1,2-Dichloroethane (0.1 M) was added AcOH (1.1 equiv.) and phenylmethanamine (1.2 equiv.). The homogenous reaction mixture was stirred at rt for 16 hrs, LC-MS indicated complete conversion of ketone to imine (MH⁺=442.0, Rt=0.68 min). To the imine solution at 0° C. was added NaBH₄ (1.4 equiv.) and the mixture was stirred at 0° C. for 2 hr. LC-MS showed still imine present. Add another 1.4 equiv NaBH₄ to the solution stir for one additional hour. Remove the ice bath and the reaction mixture was stirred at rt for 16 hrs. Quench with reaction with H₂O, diluted with EtOAc and washed with sat NaHCO₃, sat NaCl. The organic layer was dried over Na₂SO₄, filtered and concentrated. The residue was purified by flash column chromatography by ISCO Combi-flash Rf system with a Redisep column eluting with 4% MeOH/DCM to yield (+/−)-(4aS,5R,7R,8aR)-1-benzyl-4a-hydroxy-5-methyl-7-(3-nitropyridin-4-yl)hexahydro-1H-pyrano[4,3-b]pyridin-2(7H)-one in 39% yield. LC/MS (m/z): 397.9 (MH⁺), Rₜ=0.68 min. ¹H NMR (CHLOROFORM-d) δ ppm 1.27 (d, J=6.46 Hz, 3H), 1.49 (d, J=12.91 Hz, 1H), 1.91 (dd, J=14.67, 8.51 Hz, 1H), 2.16-2.28 (m, 1H), 2.54-2.65 (m, 2H), 2.68-2.81 (m, 1H), 3.29-3.38 (m, 1H), 3.55 (q, J=6.46 Hz, 1H), 3.96 (d, J=14.67 Hz, 1H), 5.08 (dd, J=10.86, 1.47 Hz, 1H), 5.35 (d, J=14.67 Hz, 1H), 7.29-7.41 (m, 5H), 7.75 (d, J=4.99 Hz, 1H), 8.84 (d, J=4.99 Hz, 1H), 9.23 (s, 1H).

### Synthesis of (4aS,5R,7R,8aR)-7-(3-aminopyridin-4-yl)-1-benzyl-5-methyloctahydro-1H-pyrano[4,3-b]pyridin-4a-ol

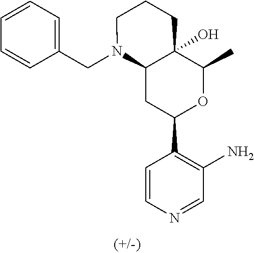 [SMILES: C[C@H]1O[C@@H](c2ccncc2N)C[C@H]2N(Cc3ccccc3)CCC[C@@]12O]

To a round-bottom flask containing (+/−)-(4aS,5R,7R,8aR)-1-benzyl-4a-hydroxy-5-methyl-7-(3-nitropyridin-4-yl)hexahydro-1H-pyrano[4,3-b]pyridin-2(7H)-one (1.0 equiv.) in THF (0.08 M) at rt was added 1 M BH₃-THF (6.6 equiv.), After stirring at rt for 90 min, the mixture was heated at 60° C. for 2 h. After cooling off to rt, the reaction was quenched with water and extracted with EtOAc. The organic layer was washed with brine, dried over Na₂SO₄, filtered and concentrated to give (+/−)-(4aS,5R,7R,8aR)-7-(3-aminopyridin-4-yl)-1-benzyl-5-methyloctahydro-1H-pyrano[4,3-b]pyridin-4a-ol in 100% yield. LC/MS (m/z): 354.0 (MH⁺), Rₜ=0.58 min.

### Synthesis of (+/−)-(4aS,5R,7R,8aR)-tert-butyl 7-(3-aminopyridin-4-yl)-4a-hydroxy-5-methyloctahydro-1H-pyrano[4,3-b]pyridine-1-carboxylate

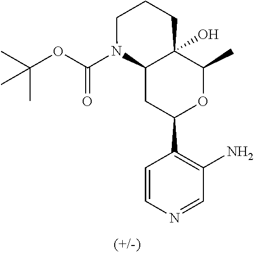 [SMILES: C[C@H]1O[C@@H](c2ccncc2N)C[C@H]2N(C(=O)OC(C)(C)C)CCC[C@@]12O]

To a solution of (+/−)-(4aS,5R,7R,8aR)-7-(3-aminopyridin-4-yl)-1-benzyl-5-methyloctahydro-1H-pyrano[4,3-b]pyridin-4a-ol (1.0 equiv.) in MeOH (0.08 M) was added 20% Pd(OH)₂ (0.3 equiv.). The reaction mixture was purged with H₂ and stirred under H₂ for 16 h. Boc anhydride (1.3 equiv.) was added and the reaction was stirred at rt for another 2 h. The mixture was filtered over celite and concentrated and purified by flash column chromatography by ISCO Combi-flash Rf system with a Redisep column eluting with 0-100% EtOAc/Heptane to yield (+/−)-(4aS,5R,7R,8aR)-tert-butyl 7-(3-aminopyridin-4-yl)-4a-hydroxy-5-methyloctahydro-1H-pyrano[4,3-b]pyridine-1-carboxylate in 30% yield. LC/MS (m/z): 364.1 (MH⁺), Rₜ=0.55 min.

### Synthesis of (4aR,5S,7S,8aS)-tert-butyl 7-(3-(6-(2,6-difluorophenyl)-5-fluoropicolinamido)pyridin-4-yl)-4a-hydroxy-5-methyloctahydro-1H-pyrano[4,3-b]pyridine-1-carboxylate and (4aS,5R,7R,8aR)-tert-butyl 7-(3-(6-(2,6-difluorophenyl)-5-fluoropicolinamido)pyridin-4-yl)-4a-hydroxy-5-methyloctahydro-1H-pyrano[4,3-b]pyridine-1-carboxylate

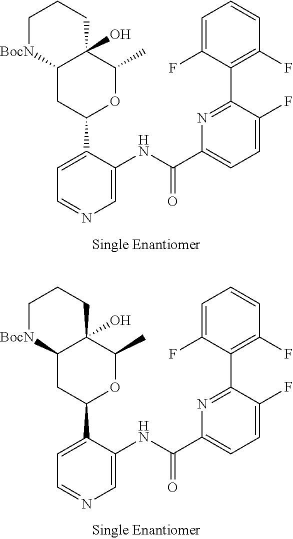

EDC (2.0 equiv.) was added to a solution of (4aS,5R,7R,8aR)-tert-butyl 7-(3-aminopyridin-4-yl)-4a-hydroxy-5-methyloctahydro-1H-pyrano[4,3-b]pyridine-1-carboxylate (1.0 equiv.), 6-(2,6-difluorophenyl)-5-fluoropicolinic acid (2.0 equiv.), and HOAt (2.0 equiv.) in DMF (0.03M). The mixture was stirred at ambient temperature overnight. The reaction mixture was diluted with water and extracted with ethyl acetate. The combined extracts were washed sequentially with 1M aqueous sodium carbonate and brine, dried over sodium sulfate, filtered, and concentrated. The crude was first purified by ISCO (50%-100% EtOAC/Heptane) and then chiral HPLC (Heptane /IPA=85/15, 20 mL/min, AD column) to yield in order of elution (4aR,5S,7S,8aS)-tert-butyl 7-(3-(6-(2,6-difluorophenyl)-5-fluoropicolinamido)pyridin-4-yl)-4a-hydroxy-5-methyloctahydro-1H-pyrano[4,3-b]pyridine-1-carboxylate (25% yield and 99% ee) and (4aS,5R,7R,8aR)-tert-butyl7-(3-(6-(2,6-difluorophenyl)-5-fluoropicolinamido)pyridin-4-yl)-4a-hydroxy-5-methyloctahydro-1H-pyrano[4,3-b]pyridine-1-carboxylate (25% yield, 99% ee). LC/MS (m/z): 599.0 (MH⁺), Rₜ=0.84 min.

### Synthesis of (3-cyclopropylideneprop-1-en-2-yloxy)triethylsilane

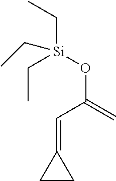

METHOD 7 was followed using 1-cyclopropylidenepropan-2-one (1.0 equiv.), TESOTf (1.0 equiv.) and Et₃N (1.4 equiv.) in 1, 2 dichlorobenzene/DCM (2/5, 0.22 M) to give (3-cyclopropylideneprop-1-en-2-yloxy)triethylsilane in 100% yield.

### Synthesis of (+/−)-(R)-3-nitro-4-(7-(triethylsilyloxy)-4-oxaspiro[2.5]oct-7-en-5-yl)pyridine

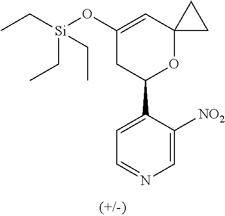 [SMILES: CC[Si](CC)(CC)OC1=CC2(CC2)O[C@@H](c2ccncc2[N+](=O)[O-])C1]

Method 8 was followed using (3-cyclopropylideneprop-1-en-2-yloxy)triethylsilane (1.0 equiv.), Eu(fod)₃ (0.05 equiv.) and 3-nitroisonicotinaldehyde (1.0 equiv.) in 1,2 dichlorobenzene (0.57 M) to yield (+/−)-(R)-3-nitro-4-(7-(triethylsilyloxy)-4-oxaspiro[2.5]oct-7-en-5-yl)pyridine in 49% yield. LC/MS (m/z): 363.1 (MH⁺), Rₜ=1.35 min. ¹H NMR (CHLOROFORM-d) δ ppm 0.0.59-0.61 (m, 1H), 0.69-0.73 (m, 6H), 0.85-0.89 (m, 1H), 0.97-1.01 (m, 9H), 1.15-1.21 (m, 1H), 2.29-2.36 (m, 1H), 2.57-2.62 (m, 1H), 4.6-4.62 (m, 1H), 5.41-5.44 (m, 1H), 7.81-7.82 (m, 1H), 9.00 (s, 1H), 9.36 (s, 1H).

### Synthesis of (+/−)-(5R,8R)-8-hydroxy-5-(3-nitropyridin-4-yl)-4-oxaspiro[2.5]octan-7-one

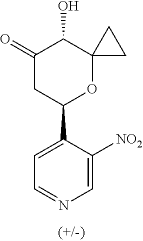 [SMILES: O=C1C[C@H](c2ccncc2[N+](=O)[O-])OC2(CC2)[C@H]1O]

METHOD 10 was followed using (+/−)-(R)-3-nitro-4-(7-(triethylsilyloxy)-4-oxaspiro[2.5]oct-7-en-5-yl)pyridine (1.0 equiv.) and 3,3-dimethyldioxirane as a solution in acetone (0.1M solution, 1.0 equiv.) in DCM (0.20 M) to give (+/−)-(5R,8R)-8-hydroxy-5-(3-nitropyridin-4-yl)-4-oxaspiro[2.5]octan-7-one in 25% yield. LC/MS (m/z): 265.0 (MH⁺), Rₜ=0.57 min.

### Synthesis of (+/−)-(5R,8R)-8-(tert-butyldimethylsilyloxy)-5-(3-nitropyridin-4-yl)-4-oxaspiro[2.5]octan-7-one

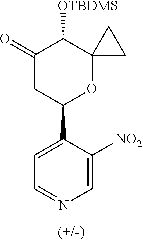 [SMILES: CC(C)(C)[Si](C)(C)O[C@H]1C(=O)C[C@H](c2ccncc2[N+](=O)[O-])OC12CC2]

To a solution of (+/−)-(5R,8R)-8-hydroxy-5-(3-nitropyridin-4-yl)-4-oxaspiro[2.5]octan-7-one (1.0 equiv.) and imidazole (4.5 equiv.) in DMF (1.13 M) was added TBDMSCl (2.2 equiv.). The solution was capped and left stirring at RT for 48 hrs. The reaction was diluted with EtOAc and was washed with H₂O, NaCl_{(sat.)}, dried over MgSO₄, filtered, concentrated. The residue was loaded onto silica gel and purified by flash chromatography over silica gel (heptanes:ethyl acetate gradient) to give (+/−)-(5R,8R)-8-(tert-butyldimethylsilyloxy)-5-(3-nitropyridin-4-yl)-4-oxaspiro[2.5]octan-7-one in 54% yield. LC/MS (m/z): 379.1 (MH⁺), Rₜ=1.26 min. ¹H NMR (400 MHz, CHLOROFORM-d) δ ppm 0.05 (s, 3H), 0.16 (s, 3H), 0.59-0.67 (m, 2H), 0.83-0.99 (m, 22H), 2.61 (ddd, J=14.09, 11.35, 1.17 Hz, 1H), 3.06 (dd, J=14.09, 2.74 Hz, 1H), 4.66 (s, 1H), 5.38 (dd, J=11.54, 2.54 Hz, 1H), 7.81 (d, J=5.09 Hz, 1H), 8.86 (d, J=5.48 Hz, 1H), 9.20 (s, 1H).

### Synthesis of (+/−)-(5R,8S)-8-(tert-butyldimethylsilyloxy)-5-(3-nitropyridin-4-yl)-4-oxaspiro[2.5]octan-7-ol

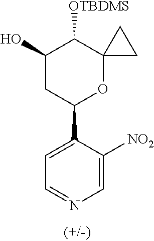 [SMILES: CC(C)(C)[Si](C)(C)O[C@H]1[C@H](O)C[C@H](c2ccncc2[N+](=O)[O-])OC12CC2]

To a stirring solution of (+/−)-(5R,8R)-8-(tert-butyldimethylsilyloxy)-5-(3-nitropyridin-4-yl)-4-oxaspiro[2.5]octan-7-one (1.0 equiv.) in EtOH (0.20 M) at −10° C. was added NaBH₄ (1.2 equiv.). The reaction was allowed to stir for 10 mins and was quenched with water. The volatiles were removed in vacuo. The residue was taken up into EtOAc and washed with brine. The organics were dried over Na₂SO₄, filtered, and concentrated to give (+/−)-(5R,8S)-8-(tert-butyldimethylsilyloxy)-5-(3-nitropyridin-4-yl)-4-oxaspiro[2.5]octan-7-ol in 99% yield. LC/MS (m/z): 381.1 (MH⁺), Rₜ=1.23 min. The product was used in next step without further purification.

### Synthesis of (+/−)-(5R,7R,8S)-8-(tert-butyldimethylsilyloxy)-5-(3-nitropyridin-4-yl)-4-oxaspiro[2.5]octan-7-yl acetate

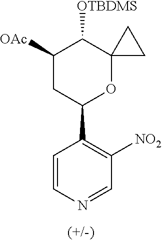 [SMILES: CC(=O)O[C@@H]1C[C@H](c2ccncc2[N+](=O)[O-])OC2(CC2)[C@H]1O[Si](C)(C)C(C)(C)C]

To a solution of (+/−)-(5R,8S)-8-(tert-butyldimethylsilyloxy)-5-(3-nitropyridin-4-yl)-4-oxaspiro[2.5]octan-7-ol (1.0 equiv.) in Pyridine (0.15 M) was added Ac₂O (5.0 equiv.). The reaction was allowed to stir at RT overnight. The reaction was quenched with water and extracted in EtOAc. The organic was washed with brine, dried over Na₂SO₄, filtered, and concentrated. The crude was loaded onto silica gel and purified by flash chromatography over silica gel (heptanes:ethyl acetate gradient) to give (+/−)-(5R,7R,8S)-8-(tert-butyldimethylsilyloxy)-5-(3-nitropyridin-4-yl)-4-oxaspiro[2.5]octan-7-yl acetate in 39% yield. LC/MS (m/z): 423.1 (MH⁺), Rₜ=1.35 min. ¹H NMR (400 MHz, CHLOROFORM-d) δ ppm 0.07-0.12 (m, 6H), 0.82-0.91 (m, 13H), 1.58-1.68 (m, 2H), 2.05-2.08 (m, 3H), 2.65 (ddd, J=12.52, 5.09, 1.96 Hz, 1H), 4.13 (d, J=9.00 Hz, 1H), 5.03 (ddd, J=10.96, 9.00, 5.09 Hz, 1H), 5.20 (dd, J=11.35, 1.96 Hz, 1H), 7.69 (d, J=5.09 Hz, 1H), 8.79 (d, J=5.09 Hz, 1H), 9.14 (s, 1H).

### Synthesis of (5S,7S,8R)-5-(3-aminopyridin-4-yl)-8-(tert-butyldimethylsilyloxy)-4-oxaspiro[2.5]octan-7-yl acetate and (5R,7R,8S)-5-(3-aminopyridin-4-yl)-8-(tert-butyldimethylsilyloxy)-4-oxaspiro[2.5]octan-7-yl acetate

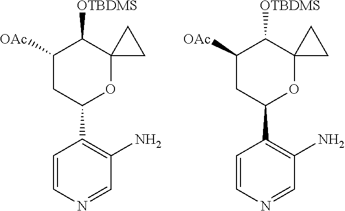 [SMILES: CC(=O)O[C@H]1C[C@@H](c2ccncc2N)OC2(CC2)[C@@H]1O[Si](C)(C)C(C)(C)C ; CC(=O)O[C@@H]1C[C@H](c2ccncc2N)OC2(CC2)[C@H]1O[Si](C)(C)C(C)(C)C]

To a solution of (+/−)-(5R,7R,8S)-8-(tert-butyldimethylsilyloxy)-5-(3-nitropyridin-4-yl)-4-oxaspiro[2.5]octan-7-yl acetate (1.0 equiv.) in degassed EtOH (0.18 M) was added 10% Pd/C (0.1 equiv.). The reaction was allowed to stir under one atm of H₂ overnight at RT, then filtered and concentrated. The crude was loaded onto silica gel and purified by flash chromatography over silica gel (heptanes:ethyl acetate gradient). Purification was completed via chiral HPLC (heptane/EtOH)=95/05, 20 mL/min, AD column) to yield in order of elution (5S,7S,8R)-5-(3-aminopyridin-4-yl)-8-(tert-butyldimethylsilyloxy)-4-oxaspiro[2.5]octan-7-yl acetate (25% yield, 99% ee) and (5R,7R,8S)-5-(3-aminopyridin-4-yl)-8-(tert-butyldimethylsilyloxy)-4-oxaspiro[2.5]octan-7-yl acetate (26% yield, 99% ee). LC/MS (m/z): 393.3 (MH⁺), Rₜ=0.94 min.

### Synthesis of 5-(3,4-dihydro-2H-pyran-6-yl)-2-methoxypyridin-4-amine

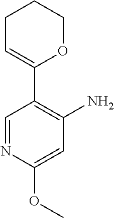

In a large microwave vial was dissolved 5-bromo-2-methoxypyridin-4-amine (1.0 equiv.), 2-(3,4-dihydro-2H-pyran-6-yl)-4,4,5,5-tetramethyl-1,3,2-dioxaborolane (2.0 equiv.) and Dichloro[1,1′-bis(di-tbutylphosphosphino)ferrocene]palladium(II) (0.1 equiv.) in DME (0.2 M). The reaction was heated in the microwave to 100° C. for 12 minutes. The reaction was concentrated in vacuo and fused to silica gel. The crude material was purified by flash chromatography over silica gel (heptanes:ethyl acetate gradient) to provide 5-(3,4-dihydro-2H-pyran-6-yl)-2-methoxypyridin-4-amine in 90% yield. LC/MS (m/z): 207.1 (MH⁺), Rₜ=0.43 min.

### Synthesis of (+/−)-2-methoxy-5-(tetrahydro-2H-pyran-2-yl)pyridin-4-amine

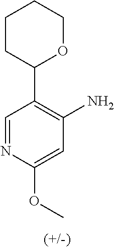

In a round bottom flask was dissolved 5-(3,4-dihydro-2H-pyran-6-yl)-2-methoxypyridin-4-amine (1.0 equiv.) in MeOH (0.12 M). To this solution was added a suspension of 10% Pd/C (0.1 equiv.) in MeOH (0.05 M) and the reaction was placed under an atmosphere of hydrogen and stirred overnight at room temperature. The reaction was filtered off over a pad of celite and washed with MeOH. The filtrated was concentrated in vacuo to brown oil. The oil was purified by prep HPLC. The fractions containing product were placed in the rotovap to remove MeCN, the neutralized with solid NaHCO₃. The aqueous phase was extracted with DCM. The combined organic layers were dried over MgSO₄, filtered, and concentrated in vacuo to provide (+/−)-2-methoxy-5-(tetrahydro-2H-pyran-2-yl)pyridin-4-amine as a clear, colorless oil in 11% yield. LC/MS (m/z): 209.1 (MH⁺), Rₜ=0.66 min.

### Synthesis of 5-((2R,3R,4R)-3,4-bis(triisopropylsilyloxy)-2-((triisopropylsilyloxy)methyl)-3,4-dihydro-2H-pyran-6-yl)-2-methoxypyridin-4-amine

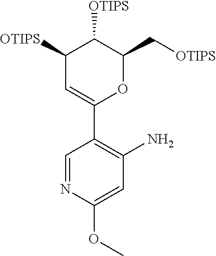

A mixture of 5-bromo-2-methoxypyridin-4-amine (1.0 equiv.), (2R,3R,4R)-3,4-bis(triisopropylsilyloxy)-2-((triisopropylsilyloxy)methyl)-3,4-dihydro-2H-pyran-6-ylboronic acid (1.5 equiv.), and aqueous (2M) Na₂CO₃ (3.0 equiv.) in DME (0.25 M) was degassed by bubbling Ar through for 5 min. PdCl₂(dppf).CH₂Cl₂ adduct (0.1 equiv.) was added, and the mixture was stirred at 90° C. overnight. The cooled reaction mixture was diluted with water and extracted with ethyl acetate. The combined extracts were dried over sodium sulfate, filtered, and concentrated. The crude product was purified by flash chromatography over silica gel (heptanes:ethyl acetate gradient) to give 5-((2R,3R,4R)-3,4-bis(triisopropylsilyloxy)-2-((triisopropylsilyloxy)methyl)-3,4-dihydro-2H-pyran-6-yl)-2-methoxypyridin-4-amine in 40% yield. LC/MS (m/z): 737.5 (MH⁺), Rₜ=1.10 min (95/95 method). ¹H NMR (400 MHz, CHLOROFORM-d) δ ppm 0.97-1.17 (m, 63H) 3.68 (d, J=10.17 Hz, 1H), 3.87 (s, 3H), 3.98 (d, J=1.57 Hz, 1H), 4.11 (d, J=5.09 Hz, 1H), 4.33-4.50 (m, 2H), 5.05 (m, 3H), 5.85 (s, 1H), 7.88 (s, 1H).

### Synthesis of 5-((2R,4R,5R,6R)-4,5-bis(triisopropylsilyloxy)-6-((triisopropylsilyloxy)methyl)tetrahydro-2H-pyran-2-yl)-2-methoxypyridin-4-amine

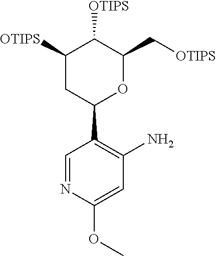

5-((2R,3R,4R)-3,4-bis(triisopropylsilyloxy)-2-((triisopropylsilyloxy)methyl)-3,4-dihydro-2H-pyran-6-yl)-2-methoxypyridin-4-amine (1.0 equiv.) was dissolved in EtOH (0.04 M). The solution was de-gassed by bubbling Ar through for 5 min. 10% palladium on carbon (0.5 equiv.) was added. The flask was purged and flushed with hydrogen twice. The reaction was stirred under a hydrogen atmosphere for 3 days. LC-MS showed the reaction was not complete. Additional 0.25 eq of palladium was added, and the mixture was stirred under H₂ for three days. The reaction mixture was diluted with DCM and methanol and filtered. The filtrate was concentrated. The crude was purified by flash chromatography over silica gel (heptanes:ethyl acetate gradient) to give 5-((2R,4R,5R,6R)-4,5-bis(triisopropylsilyloxy)-6-((triisopropylsilyloxy)methyl)tetrahydro-2H-pyran-2-yl)-2-methoxypyridin-4-amine in 35% yield. LC/MS (m/z): 739.6 (MH⁺), Rₜ=0.79 min (95/95 method). ¹H NMR (400 MHz, CHLOROFORM-d) δ ppm 1.02-1.15 (m, 63H) 2.05-2.19 (m, 1H), 2.38-2.50 (m, 1H), 3.55-3.64 (m, 1H), 3.67-3.81 (m, 2H), 3.84-3.87 (m, 3H), 4.03-4.09 (m, 2H), 4.48-4.56 (m, 1H), 4.88 (s, 2H), 5.93 (s, 1H), 7.69 (s, 1H).

### Synthesis of 4-((2R,3R,4R)-3,4-bis(triisopropylsilyloxy)-2-((triisopropylsilyloxy)methyl)-3,4-dihydro-2H-pyran-6-yl)-6-chloro-5-nitropyrimidine

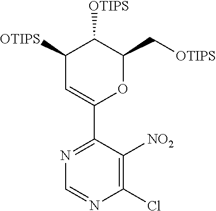

A mixture of (2R,3R,4R)-3,4-bis(triisopropylsilyloxy)-2-((triisopropylsilyloxy)methyl)-3,4-dihydro-2H-pyran-6-ylboronic acid (1.0 equiv.), 4,6-dichloro-5-nitropyrimidine (1.0 equiv.), SODIUM CARBONATE (3.0 equiv.) and Pd(PPh₃)₄ (0.02 equiv.) in Toluene/Water (5/4, 0.55 M) under argon was heated at 90° C. for 1 h. The reaction mixture was cooled to RT and diluted with water and EtOAc. The aqueous layer was separated and reextracted with EtOAc. The combined organics were dried over Na₂SO₄ and concentrated in vacuo to yield a brown oil. The oil was further purified by column chromatography eluting with a heptanes:ethyl acetate gradient to give 4-((2R,3R,4R)-3,4-bis(triisopropylsilyloxy)-2-((triisopropylsilyloxy)methyl)-3,4-dihydro-2H-pyran-6-yl)-6-chloro-5-nitropyrimidine in 49% yield. ¹H NMR (400 MHz, CHLOROFORM-d) δ ppm 1.05-1.11 (m, 63H), 3.84-3.93 (m, 1H), 3.95-4.03 (m, 1H), 4.25 (m, 2H), 4.39 (m, 1H), 6.44-6.54 (m, 1H), 8.93 (s, 1H).

### Synthesis of ((2R,3R,4R)-6-(6-chloro-5-nitropyrimidin-4-yl)-3,4-bis(triisopropylsilyloxy)-3,4-dihydro-2H-pyran-2-yl)methanol

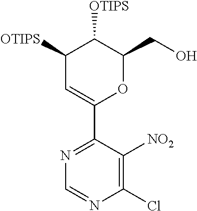

To a solution of 4-((2R,3R,4R)-3,4-bis(triisopropylsilyloxy)-2-((triisopropylsilyloxy)methyl)-3,4-dihydro-2H-pyran-6-yl)-6-chloro-5-nitropyrimidine (1.0 equiv.) in THF (0.15 M) was added 37% Hydrochloric acid (6.0 equiv.). The mixture was stirred at ambient temperature for 7 hr. The reaction mixture was cooled in an ice water bath, neutralized with saturated aqueous sodium bicarbonate, and extracted with ethyl acetate. The combined organic layers were dried over sodium sulfate, filtered, and concentrated. The crude material was purified by flash chromatography (heptanes:ethyl acetate gradient) to give ((2R,3R,4R)-6-(6-chloro-5-nitropyrimidin-4-yl)-3,4-bis(triisopropylsilyloxy)-3,4-dihydro-2H-pyran-2-yl)methanol in 50% yield. ¹H NMR (400 MHz, CHLOROFORM-d) δ ppm 1.00-1.14 (m, 42H), 3.57-3.70 (m, 1H), 3.95-4.06 (m, 1H), 4.12 (d, J=1.57 Hz, 1H), 4.20-4.28 (m, 1H), 4.40-4.49 (m, 1H), 6.54 (dd, J=5.48, 1.57 Hz, 1H), 8.96 (s, 1H).

### Synthesis of (2S,3R,4R)-6-(6-chloro-5-nitropyrimidin-4-yl)-3,4-bis(triisopropylsilyloxy)-3,4-dihydro-2H-pyran-2-carbaldehyde

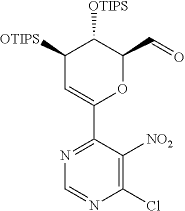

((2R,3R,4R)-6-(6-chloro-5-nitropyrimidin-4-yl)-3,4-bis(triisopropylsilyloxy)-3,4-dihydro-2H-pyran-2-yl)methanol (1.0 equiv.) was dissolved in DCM (0.13 M). Dess-Martin Periodinane (1.5 equiv.) was added at ambient temperature. The reaction was allowed to proceed for a total of 3 hrs. The reaction mixture was diluted with DCM and quenched with saturated aqueous sodium bicarbonate. After stirring for 10 min, the mixture was filtered through Celite. The filtrate layers were separated. The filter cake was rinsed with additional DCM. The aqueous phase was extracted with the second filtrate. The combined organic layers were dried over sodium sulfate, filtered, and concentrated with silica gel. The crude material was purified by flash chromatography over silica gel (heptanes:ethyl acetate gradient) to give (2S,3R,4R)-6-(6-chloro-5-nitropyrimidin-4-yl)-3,4-bis(triisopropylsilyloxy)-3,4-dihydro-2H-pyran-2-carbaldehyde in 55% yield. ¹H NMR (400 MHz, CHLOROFORM-d) δ ppm 1.00-1.16 (m, 42H), 4.25 (m, 1H), 4.39 (m, 1H), 4.61 (m, 1H), 6.66 (d, J=5.87 Hz, 1H), 8.99 (s, 1H) 9.47 (s, 1H).

### Synthesis of 4-((2R,3R,4R)-3,4-bis(triisopropylsilyloxy)-2-vinyl-3,4-dihydro-2H-pyran-6-yl)-6-chloro-5-nitropyrimidine

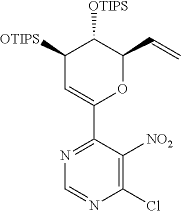

To a solution of POTASSIUM TERT-BUTOXIDE (1.5 equiv.) in THF (0.27 M) was added METHYLTRIPHENYLPHOSPHONIUM BROMIDE (1.5 equiv.) at ambient temperature. The yellow mixture was stirred at 50° C. for 20 min and then returned to ambient temperature. A solution of (2S,3R,4R)-6-(6-chloro-5-nitropyrimidin-4-yl)-3,4-bis(triisopropylsilyloxy)-3,4-dihydro-2H-pyran-2-carbaldehyde (1.0 equiv.) in THF (0.36 M) was added in a dropwise fashion. After 30 min, the reaction was quenched by the addition of saturated aqueous sodium bicarbonate and extracted with ethyl acetate. The combined extracts were dried over sodium sulfate, filtered, and concentrated with silica gel. The crude mixture was concentrated and purified by flash chromatography (heptanes:ethyl acetate gradient) to give 4-((2R,3R,4R)-3,4-bis(triisopropylsilyloxy)-2-vinyl-3,4-dihydro-2H-pyran-6-yl)-6-chloro-5-nitropyrimidine in 30% yield. ¹H NMR (400 MHz, CHLOROFORM-d) δ ppm 1.00-1.16 (m, 42H), 4.09 (d, J=1.57 Hz, 1H), 4.25 (br. s., 1H), 4.67-4.77 (m, 1H), 5.10-5.28 (m, 2H), 6.03-6.19 (m, 1H), 6.56 (d, J=3.91 Hz, 1H), 8.94 (s, 1H).

### Synthesis of 4-((2S,4R,5R,6R)-6-ethyl-4,5-bis(triisopropylsilyloxy)tetrahydro-2H-pyran-2-yl)pyrimidin-5-amine

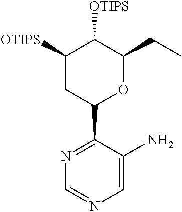

To a degassed solution of 4-((2R,3R,4R)-3,4-bis(triisopropylsilyloxy)-2-vinyl-3,4-dihydro-2H-pyran-6-yl)-6-chloro-5-nitropyrimidine (1.0 equiv.) in EtOH (0.03 M) was added 10% PALLADIUM ON CARBON (0.30 equiv.). The flask was purged and flushed twice with hydrogen. The reaction was stirred under a hydrogen balloon for 2 days. The reaction mixture was diluted with methanol and DCM and filtered through Celite. The filtrate was concentrated and the crude product was purified by flash chromatography (heptanes:ethyl acetate gradient) to give 4-((2S,4R,5R,6R)-6-ethyl-4,5-bis(triisopropylsilyloxy)tetrahydro-2H-pyran-2-yl)pyrimidin-5-amine in 35% yield. LC/MS (m/z): 552.3 (MH⁺), Rₜ=0.64 min (95/95 method).

### Synthesis of ((2R,3R,4R)-6-(3-nitropyridin-4-yl)-3,4-bis((triisopropylsilyl)oxy)-3,4-dihydro-2H-pyran-2-yl)methanol

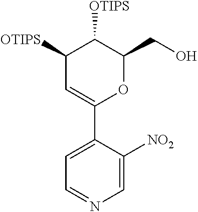

A solution 4-((2R,3R,4R)-3,4-bis((triisopropylsilyl)oxy)-2-(((triisopropylsilyl)oxy)methyl)-3,4-dihydro-2H-pyran-6-yl)-3-nitropyridine (1.0 equiv.) in THF (0.11 M) was cooled in an ice-water bath. 37% Hydrochloric acid (5.0 equiv.) was added in a dropwise fashion. The mixture was stirred, allowing to come to ambient temperature, for 4.5 hrs. The reaction mixture was cooled in an ice-water bath, neutralized with saturated aqueous sodium bicarbonate, and extracted with ethyl acetate. The combined organic layers were dried over sodium sulfate, filtered, and concentrated. The crude material was purified by flash chromatography (heptanes:ethyl acetate gradient) to give ((2R,3R,4R)-6-(3-nitropyridin-4-yl)-3,4-bis((triisopropylsilyl)oxy)-3,4-dihydro-2H-pyran-2-yl)methanol in 48% yield. LC/MS (m/z): 581.3 (MH⁺), Rₜ=0.61 min. ¹H NMR (400 MHz, CHLOROFORM-d) δ ppm 1.03-1.11 (m, 42H), 2.40-2.50 (m, 1H), 3.60-3.70 (m, 1H), 4.07-4.28 (m, 3H), 4.40-4.47 (m, 1H), 5.36 (dd, J=5.67, 1.37 Hz, 1H), 7.45 (d, J=5.09 Hz, 1H), 8.78 (d, J=5.09 Hz, 1H), 8.97 (s, 1H).

### Synthesis of ((2R,3R,4R)-6-(3-nitropyridin-4-yl)-3,4-bis((triisopropylsilyl)oxy)-3,4-dihydro-2H-pyran-2-yl)methyl acetate

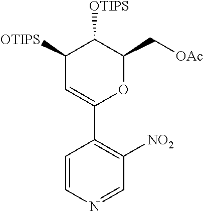

To a solution of ((2R,3R,4R)-6-(3-nitropyridin-4-yl)-3,4-bis((triisopropylsilyl)oxy)-3,4-dihydro-2H-pyran-2-yl)methanol (1.0 equiv.) in pyridine (0.17 M) was added acetic anhydride (5.0 equiv.) and the reaction was stirred at room temperature for 4 h. Upon completion, the volatiles were removed under vacuo, the crude was dissolved in ethyl acetate and washed with water. The organic phase was dried with sodium sulfate, filtered and concentrated to yield ((2R,3R,4R)-6-(3-nitropyridin-4-yl)-3,4-bis((triisopropylsilyl)oxy)-3,4-dihydro-2H-pyran-2-yl)methyl acetate in 100% yield. LC/MS (m/z): 623.2 (MH⁺), Rₜ=0.73 min (95/95 method). The crude was used for the next step without further purification.

### Synthesis of ((2R,3R,4R,6R)-6-(3-aminopyridin-4-yl)-3,4-bis((triisopropylsilyl)oxy)tetrahydro-2H-pyran-2-yl)methyl acetate

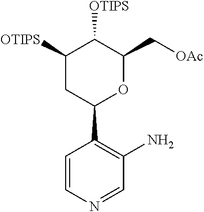

To a degassed solution of ((2R,3R,4R)-6-(3-nitropyridin-4-yl)-3,4-bis((triisopropylsilyl)oxy)-3,4-dihydro-2H-pyran-2-yl)methyl acetate (1.0 equiv.) in EtOH (0.17 M) was added 10% Pd/C (0.1 equiv.) and the reaction was stirred under a hydrogen balloon for 40 hrs. The reaction was filtered through a pad of Celite and washed with ethyl acetate. The filtrate was concentrated to yield ((2R,3R,4R,6R)-6-(3-aminopyridin-4-yl)-3,4-bis((triisopropylsilyl)oxy)tetrahydro-2H-pyran-2-yl)methyl acetate in 93% yield and used for the next step without further purification. LC/MS (m/z): 595.2 (MH⁺), Rₜ=1.06 min.

### Synthesis of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)methyl acetate

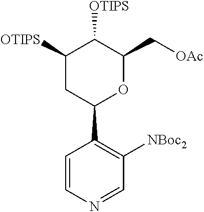

To a solution of ((2R,3R,4R,6R)-6-(3-aminopyridin-4-yl)-3,4-bis((triisopropylsilyl)oxy)tetrahydro-2H-pyran-2-yl)methyl acetate (1.0 equiv.) in DCM (0.16 M) was added boc-anhydride (2.7 equiv.) and DMAP (0.1 equiv.). The reaction was stirred at room temperature overnight. The reaction was quenched by the addition of water; the organic phase was dried with sodium sulfate, filtered and concentrated. The crude material was purified via silica gel column chromatography eluting with ethyl acetate and heptanes (0-50% ethyl acetate ramp over 10 min) to yield ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)methyl acetate in 47% yield. LC/MS (m/z): 795.5 (MH⁺), Rₜ=0.53 min (95/95 method). ¹H NMR (400 MHz, CHLOROFORM-d) δ ppm 0.99-1.19 (m, 42H), 1.31-1.47 (m, 9H), 1.70 (ddd, J=13.60, 10.86, 7.24 Hz, 1H), 1.98-2.08 (s, 3H), 2.30 (ddd, J=13.30, 5.48, 3.91 Hz, 1H), 3.63-3.73 (m, 1H), 3.82 (t, J=6.06 Hz, 1H), 4.00-4.10 (m, 1H), 4.28 (dd, J=11.54, 6.06 Hz, 1H), 4.37 (dd, J=11.35, 3.91 Hz, 1H), 4.66 (dd, J=10.56, 3.52 Hz, 1H), 7.50 (d, J=5.48 Hz, 1H), 8.29 (s, 1H), 8.54 (d, J=5.09 Hz, 1H).

### Synthesis of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-methanol

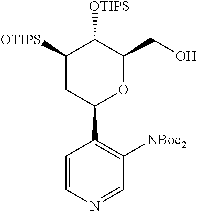

To a solution of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)methyl acetate (1.0 equiv.) in MeOH (0.15 M) was added potassium carbonate (2.0 equiv.). The reaction was stirred at room temperature for 3 h, quenched by the addition of water and extracted with DCM. The aqueous phase was extracted with DCM twice until no product in aqueous phase. The organics were combined, dried with sodium sulfate, filtered and concentrated to yield ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-methanol in 79% yield. LC/MS (m/z): 753.5 (MH⁺), Rₜ=0.50 min (95/95 method). ¹H NMR (400 MHz, CHLOROFORM-d) δ ppm 1.05-1.14 (m, 42H), 1.40 (d, J=5.87 Hz, 18H), 1.73-1.86 (m, 1H), 2.28 (ddd, J=13.40, 5.18, 3.33 Hz, 1H), 2.79 (t, J=6.65 Hz, 1H), 3.40-3.48 (m, 1H), 3.75-3.86 (m, 2H), 3.97-4.06 (m, 1H), 4.67 (dd, J=10.96, 3.13 Hz, 1H), 7.23-7.32 (m, 1H), 8.32 (s, 1H), 8.53 (d, J=5.09 Hz, 1H).

### Synthesis of ((2S,3R,4R,6R)-6-(3-((bis-tert-butoxycarbonyl)amino)pyridin-4-yl)-3,4-bis((triisopropylsilyl)oxy)tetrahydro-2H-pyran-2-yl)-carboxaldehyde

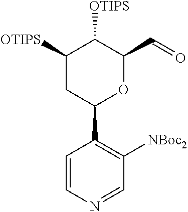

To a solution of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-methanol (1.0 equiv.) in DCM (0.11 M) at 0° C. was added sodium bicarbonate (2.0 equiv.) and DMP (1.5 equiv.). The reaction was allowed to warm to room temperature and stirred for 3 h. The reaction was quenched with sat. sodium bicarbonate and extracted with DCM. The organic phase was dried with sodium sulfate, filtered and concentrated under vacuo. The crude material was purified via silica gel column chromatography (ISCO eluting with hexanes and ethyl acetate—0-30% ethyl acetate) to give ((2S,3R,4R,6R)-6-(3-((bis-tert-butoxycarbonyl)amino)pyridin-4-yl)-3,4-bis((triisopropylsilyl)oxy)tetrahydro-2H-pyran-2-yl)-carboxaldehyde as a yellow oil in 78% yield. ¹H NMR (400 MHz, CHLOROFORM-d) δ ppm 1.05-1.16 (m, 42H), 1.34 (s, 9H) 1.37-1.42 (m, 9H), 1.75 (dd, J=14.09, 10.17 Hz, 1H), 2.33-2.43 (m, 1H), 4.17-4.33 (m, 3H), 5.20 (dd, J=9.98, 6.06 Hz, 1H), 7.68 (d, J=5.09 Hz, 1H), 8.31 (s, 1H), 8.60 (d, J=5.09 Hz, 1H), 9.75 (s, 1H).

### Synthesis of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-ethylene

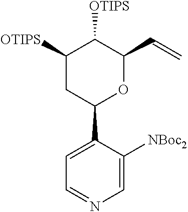

To a solution of methyltriphenylphosphonium bromide (1.5 equiv.) in THF (0.1 M) was added slowly LITHIUM BIS(TRIMETHYLSILYL)AMIDE (1.5 equiv.) at 0° C. The cooling bath was removed and the ylide solution was stirred for 1 hr allowing the r×n to warm to room temp. The r×n was again cooled to 0° C. and ((2S,3R,4R,6R)-6-(3-((bis-tert-butoxycarbonyl)amino)pyridin-4-yl)-3,4-bis((triisopropylsilyl)oxy)tetrahydro-2H-pyran-2-yl)-carboxaldehyde (1.0 equiv.) in THF (0.1 M) was added to the ylide solution. After addition, the cooling bath was removed and the r×n was allowed to stir for 2 h. The reaction was quenched by the addition of water and extracted with ethyl acetate. The organic phase was dried with sodium sulfate, filtered and concentrated. The crude material was purified via silica gel column chromatography eluting with ethyl acetate and heptanes (0-30% ethyl acetate) to give ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-ethylene in 57% yield. LC/MS (m/z): 749.4 (MH⁺), Rₜ=0.70 min (95/95 method).

### Synthesis of 4-((2R,4R,5R,6R)-4,5-bis((triisopropylsilyl)oxy)-6-vinyltetrahydro-2H-pyran-2-yl)pyridin-3-amine

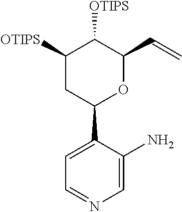

To a solution of ((2R,3R,4R,6R)-6-(3-((bis-tert-butoxycarbonyl)amino)pyridin-4-yl)-3,4-bis((triisopropylsilyl)oxy)tetrahydro-2H-pyran-2-yl)-ethylene (1.0 equiv.) in DCM (0.04 M) was added TFA (160.0 equiv.). The reaction was stirred at room temperature for 2 h, concentrated under vacuo, then partitioned between ethyl acetate and sat. NaHCO₃. The organic phase was dried with sodium sulfate, filtered and concentrated to give 4-((2R,4R,5R,6R)-4,5-bis((triisopropylsilyl)oxy)-6-vinyltetrahydro-2H-pyran-2-yl)pyridin-3-amine in 100% yield. LC/MS (m/z): 549.3 (MH⁺), Rₜ=1.20 min (65/95 method). The crude material was used for the next step without further optimization.

### Synthesis of (E)-ethyl 3-((2R,3R,4R)-6-(3-nitropyridin-4-yl)-3,4-bis(triisopropylsilyloxy)-3,4-dihydro-2H-pyran-2-yl)acrylate

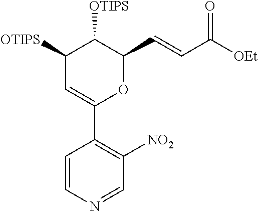

To a suspension of 60% sodium hydride (2.0 equiv.) in DME (0.07 M) was added triethyl phosphonoacetate (2.1 equiv.). After stirring at rt for 1 hr, the mixture was cooled in an ice water bath. (2S,3R,4R)-6-(3-nitropyridin-4-yl)-3,4-bis(triisopropylsilyloxy)-3,4-dihydro-2H-pyran-2-carbaldehyde (1.0 equiv.) was added. The mixture was stirred at 0 C for 30 min. The reaction was quenched by the addition of 1M acetic acid in methanol. After stirring for 5 min, the mixture was concentrated and purified by flash chromatography (heptanes:ethyl acetate gradient) to give (E)-ethyl 3-((2R,3R,4R)-6-(3-nitropyridin-4-yl)-3,4-bis(triisopropylsilyloxy)-3,4-dihydro-2H-pyran-2-yl)acrylate as a yellow oil in 99% yield. LC/MS (m/z): 649.4 (MH⁺), Rₜ=0.83 min. ¹H NMR (400 MHz, CHLOROFORM-d) δ ppm 1.02-1.12 (m, 42H) 1.27 (t, J=7.04 Hz, 3H) 4.05-4.26 (m, 4H) 4.88 (d, J=5.87 Hz, 1H) 5.43 (d, J=4.70 Hz, 1H) 5.88 (dd, J=15.65, 1.17 Hz, 1H) 7.15 (dd, J=15.85, 6.85 Hz, 1H) 7.44 (d, J=5.09 Hz, 1H) 8.77 (d, J=5.09 Hz, 1H) 8.95 (s, 1H).

### Synthesis of ethyl 3-((2R,3R,4R,6S)-6-(3-aminopyridin-4-yl)-3,4-bis(triisopropylsilyloxy)tetrahydro-2H-pyran-2-yl)propanoate

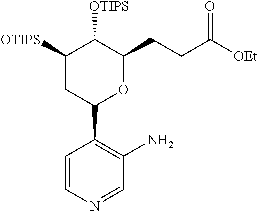

To a degassed solution of (E)-ethyl 3-((2R,3R,4R)-6-(3-nitropyridin-4-yl)-3,4-bis(triisopropylsilyloxy)-3,4-dihydro-2H-pyran-2-yl)acrylate (1.0 equiv.) in EtOH (0.15 M) was added 10% Pd/C (0.1 equiv.) and the reaction was stirred under a hydrogen balloon for 22 hrs. The mixture was filtered through a pad of Celite and washed with ethyl acetate. The filtrate was concentrated to dryness and the reaction was set up with 10% Pd/C (0.1 equiv.) in EtOH (0.08 M) under a hydrogen balloon. After overnight stirring, the reaction was complete, filtered through Celite and washed with ethyl acetate and concentrated the filtrate to afford ethyl 3-((2R,3R,4R,6S)-6-(3-aminopyridin-4-yl)-3,4-bis(triisopropylsilyloxy)tetrahydro-2H-pyran-2-yl)propanoate oil in 76% yield. LC/MS (m/z): 623.3 (MH⁺), Rₜ=1.16 min.

### Synthesis of ethyl 3-((2R,3R,4R,6S)-6-(3-(bis(tert-butoxycarbonyl)amino)pyridin-4-yl)-3,4-bis(triisopropylsilyloxy)tetrahydro-2H-pyran-2-yl)propanoate

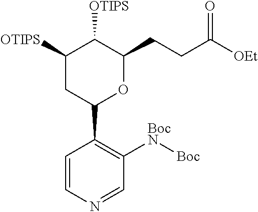

To a solution of ethyl 3-((2R,3R,4R,6S)-6-(3-aminopyridin-4-yl)-3,4-bis(triisopropylsilyloxy)tetrahydro-2H-pyran-2-yl)propanoate (1.0 equiv.) in DCM (0.12 M) was added DMAP (0.1 equiv.) and Boc anhydride (2.5 equiv.). The reaction was stirred at room temperature for 3 h. Checked reaction by LC/MS, small amount of product, but mostly starting material. Added another 1.5 equiv. of Boc₂O and another 0.1 equiv. of DMAP and allowed to stir overnight. The reaction was concentrated to dryness and purified via silica gel column chromatography (ISCO, 24 g column, eluting with ethyl acetate and heptanes 0-30% ethyl acetate ramp for 5 min, hold at 30% for 5 min). The fractions were concentrated to yield ethyl 3-((2R,3R,4R,6S)-6-(3-(bis(tert-butoxycarbonyl)amino)pyridin-4-yl)-3,4-bis(triisopropylsilyloxy)tetrahydro-2H-pyran-2-yl)propanoate as an orange oil in 72% yield. LC/MS (m/z): 823.6 (MH⁺), Rₜ=0.53 min (95/95-method). ¹H NMR (400 MHz, CHLOROFORM-d) δ ppm 1.00-1.15 (m, 42H), 1.20 (t, J=7.14 Hz, 3H), 1.37 (s, 9H), 1.42 (s, 9H), 1.59-1.67 (m, 1H), 1.92-2.06 (m, 1H), 2.11-2.22 (m, 1H), 2.23-2.37 (m, 2H), 2.51 (ddd, J=15.85, 9.59, 5.87 Hz, 1H), 3.33-3.41 (m, 1H), 3.59 (t, J=6.36 Hz, 1H), 3.93-4.02 (m, 1H), 4.07 (qd, J=7.11, 1.76 Hz, 2H), 4.54 (dd, J=10.76, 3.33 Hz, 1H), 7.50 (d, J=5.09 Hz, 1H), 8.27 (s, 1H), 8.52 (d, J=5.09 Hz, 1H).

### Synthesis of ethyl 3-((2R,3R,4R,6S)-6-(3-(bis(tert-butoxycarbonyl)amino)pyridin-4-yl)-3,4-bis(triisopropylsilyloxy)tetrahydro-2H-pyran-2-yl)propanoate

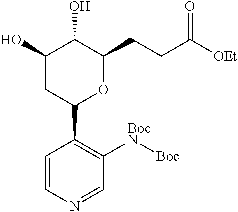

To a solution of ethyl 3-((2R,3R,4R,6S)-6-(3-(bis(tert-butoxycarbonyl)amino)pyridin-4-yl)-3,4-bis(triisopropylsilyloxy)tetrahydro-2H-pyran-2-yl)propanoate (1.0 equiv.) in THF (0.08 M) at room temperature was added TBAF (2.5 equiv.) and the reaction was stirred at room temperature for 2 h. Upon completion as judged by TLC and UPLC, The reaction was worked up by the addition of water and extracted with ethyl acetate. The organics were combined, dried with sodium sulfate, filtered and concentrated. The crude material was purified via silica gel column chromatography eluting with ethyl acetate and heptanes (ISCO, 24 g column, 0-100% ethyl acetate ramp in 5 min, hold at 100% for 5 min). The pure fractions were concentrated to give ethyl 3-((2R,3R,4R,6S)-6-(3-(bis(tert-butoxycarbonyl)amino)pyridin-4-yl)-3,4-bis(triisopropylsilyloxy)tetrahydro-2H-pyran-2-yl)propanoate as a yellow foam in 76% yield. LC/MS (m/z): 511.1 (MH⁺), Rₜ=0.69 min.

### Synthesis of ethyl 3-((2R,3R,4R,6S)-6-(3-(bis(tert-butoxycarbonyl)amino)pyridin-4-yl)-4-(tert-butyldimethylsilyloxy)-3-hydroxytetrahydro-2H-pyran-2-yl)propanoate

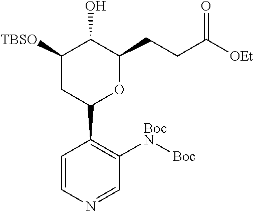

To a solution of ethyl 3-((2R,3S,4R,6S)-6-(3-(bis(tert-butoxycarbonyl)amino)pyridin-4-yl)-3,4-dihydroxytetrahydro-2H-pyran-2-yl)propanoate (1.0 equiv.) in DMF (0.13 M) at 0° C. was added imidazole (2.1 equiv.) followed by TBDMSCl (1.2 equiv.). The reaction was stirred at 0° C. under nitrogen-allowed to warm to room temperature overnight. Added another 1.0 equiv. of TBSCl and stir for another 6 h. Quenched by the addition of water and extracted with ethyl acetate. The organics were combined, dried with sodium sulfate, filtered and concentrated. The crude material was purified via silica gel column chromatography (ISCO, eluting with ethyl acetate and heptanes) to give ethyl 3-((2R,3R,4R,6S)-6-(3-(bis(tert-butoxycarbonyl)amino)pyridin-4-yl)-4-(tert-butyldimethylsilyloxy)-3-hydroxytetrahydro-2H-pyran-2-yl)propanoate in 80% yield. LC/MS (m/z): 625.0 (MH⁺), Rₜ=1.17 min. ¹H NMR (400 MHz, CHLOROFORM-d) δ ppm 0.07 (s, 3H) 0.12 (s, 3H) 0.87 (s, 9H) 1.22 (t, J=7.24 Hz, 3H) 1.37 (s, 9H) 1.41 (s, 9H) 1.88-2.01 (m, 1H) 2.05 (ddd, J=13.21, 4.99, 2.15 Hz, 1H) 2.16-2.29 (m, 1H) 2.33-2.45 (m, 2H) 2.47-2.59 (m, 1H) 3.16-3.37 (m, 2H) 3.67 (ddd, J=11.15, 8.02, 5.09 Hz, 1H) 4.10 (qd, J=7.11, 0.98 Hz, 2H) 4.46 (dd, J=11.54, 1.76 Hz, 1H) 7.46 (d, J=5.09 Hz, 1H) 8.29 (s, 1H) 8.53 (d, J=5.09 Hz, 1H)

### Synthesis of ethyl 3-((2R,4R,6S)-6-(3-(bis(tert-butoxycarbonyl)amino)pyridin-4-yl)-4-(tert-butyldimethylsilyloxy)-3-oxotetrahydro-2H-pyran-2-yl)propanoate

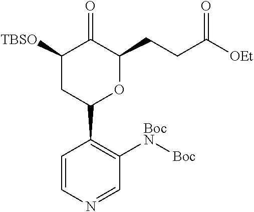

To a solution of ethyl 3-((2R,3R,4R,6S)-6-(3-(bis(tert-butoxycarbonyl)amino)pyridin-4-yl)-4-(tert-butyldimethylsilyloxy)-3-hydroxytetrahydro-2H-pyran-2-yl)propanoate (1.0 equiv.) in DCM (0.10 M) at room temperature was added sodium bicarbonate (3.0 equiv.) followed by DMP (1.5 equiv.). The reaction was stirred at room temperature for 1 hr. Quenched by the addition of water and extracted 3 times with DCM. The organics were combined, dried with sodium sulfate, filtered and concentrated. The crude material was purified via silica gel column chromatography eluting with ethyl acetate and heptanes (0-30% ethyl acetate ramp, hold at 30% until elution of product) to give ethyl 3-((2R,4R,6S)-6-(3-(bis(tert-butoxycarbonyl)amino)pyridin-4-yl)-4-(tert-butyldimethylsilyloxy)-3-oxotetrahydro-2H-pyran-2-yl)propanoate in 78% yield. LC/MS (m/z): 623.4 (MH⁺), Rₜ=1.26 min. ¹H NMR (400 MHz, CHLOROFORM-d) δ ppm 0.00 (s, 6H), 0.88 (s, 9H), 1.22 (t, J=7.14 Hz, 3H), 1.37 (s, 9H), 1.41 (s, 9H), 1.97-2.15 (m, 2H), 2.24 (dtd, J=14.87, 7.53, 7.53, 4.50 Hz, 1H), 2.38-2.47 (m, 2H), 2.53 (ddd, J=13.30, 7.04, 1.96 Hz, 1H), 3.98-4.17 (m, 3H), 4.33-4.48 (m, 1H), 4.93 (dd, J=11.74, 1.96 Hz, 1H), 7.45 (d, J=5.09 Hz, 1H), 8.34 (s, 1H), 8.56 (d, J=5.28 Hz, 1H).

### Synthesis of (2R,4R,4aR,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one and (2R,4R,4aS,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one

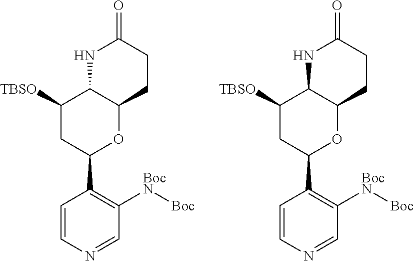 [SMILES: CC(C)(C)OC(=O)N(C(=O)OC(C)(C)C)c1cnccc1[C@H]1C[C@@H](O[Si](C)(C)C(C)(C)C)[C@@H]2NC(=O)CC[C@H]2O1 ; CC(C)(C)OC(=O)N(C(=O)OC(C)(C)C)c1cnccc1[C@H]1C[C@@H](O[Si](C)(C)C(C)(C)C)[C@H]2NC(=O)CC[C@H]2O1]

To a solution of ethyl 3-((2R,4R,6S)-6-(3-(bis(tert-butoxycarbonyl)amino)pyridin-4-yl)-4-(tert-butyldimethylsilyloxy)-3-oxotetrahydro-2H-pyran-2-yl)propanoate (1.0 equiv.) in MeOH (0.08 M) was added ammonium acetate (40.0 equiv.) and sodium cyanoborohydride (10.0 equiv.). The reaction was stirred at room temperature for 7 hrs. The reaction was worked up by removing the solvents under vacuo and partitioning the crude between ethyl acetate and water. The organic phase was dried with sodium sulfate, filtered and concentrated. The crude material was purified via silica gel column chromatography (ISCO, eluting with DCM/MeOH (10%)) to give a 1:1 mixture of inseparable (2R,4R,4aR,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one and (2R,4R,4aS,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one in 75% yield. LC/MS (m/z): 578.3 (MH⁺), Rₜ=1.02 min.

### Synthesis of (2S,4R,8aR)-2-(3-aminopyridin-4-yl)-4-(tert-butyldimethylsilyloxy)hexahydro-2H-pyrano[3,2-b]pyridin-6(7H)-one

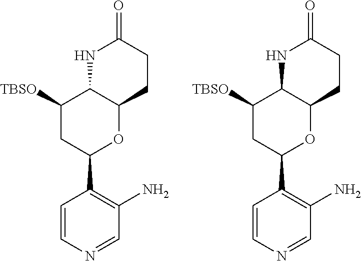 [SMILES: CC(C)(C)[Si](C)(C)O[C@@H]1C[C@H](c2ccncc2N)O[C@@H]2CCC(=O)N[C@H]21 ; CC(C)(C)[Si](C)(C)O[C@@H]1C[C@H](c2ccncc2N)O[C@@H]2CCC(=O)N[C@@H]21]

To a solution of (2R,4R,4aR,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one and (2R,4R,4aS,8aR)-2-(3-[bis-(tert-butyl-oxycarbonyl)]-aminopyridin-4-yl)-4-((tert-butyldimethylsilyl)oxy)hexahydro-2H-pyrano[3,2-b]pyridin-6(7H)-one (1.0 equiv., 1:1 mixture) in DCM (0.06 M) was added TFA (55.0 equiv.) at room temperature and the reaction was stirred for 2 h. The reaction was quenched by the addition of sat. NaHCO₃, then diluted with more DCM and extracted the organic phase. The organic layer was dried with sodium sulfate, filtered and concentrated to give a 1:1 mixture of inseparable (2S,4R,8aR)-2-(3-aminopyridin-4-yl)-4-(tert-butyldimethylsilyloxy)hexahydro-2H-pyrano[3,2-b]pyridin-6(7H)-one in 98% yield. LC/MS (m/z): 378.1 (MH⁺), Rₜ=0.66, 0.69 min. The diastereomers were separated via prep-HPLC at the final product stage.

### Synthesis of (2R,3S,4R)-6-(3-nitropyridin-4-yl)-2-vinyl-3,4-dihydro-2H-pyran-3,4-diol

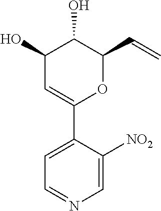 [SMILES: C=C[C@H]1OC(c2ccncc2[N+](=O)[O-])=C[C@@H](O)[C@@H]1O]

4-((2R,3R,4R)-3,4-bis(triisopropylsilyloxy)-2-vinyl-3,4-dihydro-2H-pyran-6-yl)-3-nitropyridine (1.0 equiv.) was dissolved in THF (0.13 M). A 1.0 M THF solution of TBAF (3.0 equiv.) was added at ambient temperature. The mixture was stirred overnight. The reaction mixture was diluted with ethyl acetate and washed twice with water. The organic phase was dried over sodium sulfate, filtered, and concentrated. The crude material was purified by flash chromatography (heptanes:ethyl acetate gradient) to give (2R,3S,4R)-6-(3-nitropyridin-4-yl)-2-vinyl-3,4-dihydro-2H-pyran-3,4-diol in 58.3% yield. LC/MS (m/z): 265.0 (MH⁺), Rₜ=0.49 min. ¹H NMR (400 MHz, CHLOROFORM-d) δ ppm 9.00 (s, 1H), 8.81 (d, J=5.09 Hz, 1H), 7.67 (d, J=4.70 Hz, 1H), 5.92-6.02 (m, 1H), 5.50 (d, J=2.74 Hz, 1H), 5.41 (d, J=6.26 Hz, 1H), 5.32 (d, J=5.87 Hz, 1H), 5.24 (t, J=1.56 Hz, 1H), 5.22 (d, J=1.57 Hz, 1H), 5.19-5.21 (m, 1H), 4.06-4.18 (m, 1H).

### Synthesis of (2R,3R,4R)-4-(tert-butyldimethylsilyloxy)-6-(3-nitropyridin-4-yl)-2-vinyl-3,4-dihydro-2H-pyran-3-ol

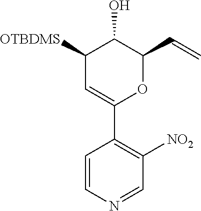 [SMILES: C=C[C@H]1OC(c2ccncc2[N+](=O)[O-])=C[C@@H](O[Si](C)(C)C(C)(C)C)[C@@H]1O]

(2R,3S,4R)-6-(3-nitropyridin-4-yl)-2-vinyl-3,4-dihydro-2H-pyran-3,4-diol (1.0 equiv.) and imidazole (2.0 equiv.) were dissolved in DMF (0.35 M) and cooled to 0° C. TBDMS-Cl (1.1 equiv.) was added. The mixture was stirred for 44 hr, allowing to come to rt. The reaction mixture was diluted with water and extracted with ethyl acetate. The combined organics were dried over sodium sulfate, filtered, and concentrated. The crude product was purified by flash chromatography over silica gel (heptanes:ethyl acetate gradient) to give (2R,3R,4R)-4-(tert-butyldimethylsilyloxy)-6-(3-nitropyridin-4-yl)-2-vinyl-3,4-dihydro-2H-pyran-3-ol in 82% yield. LC/MS (m/z): 379.1 (MH⁺), Rₜ=1.13 min.

### Synthesis of (2R,3R,4R)-4-(tert-butyldimethylsilyloxy)-6-(3-nitropyridin-4-yl)-2-vinyl-3,4-dihydro-2H-pyran-3-yl trifluoromethanesulfonate

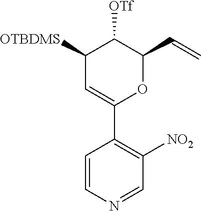 [SMILES: C=C[C@H]1OC(c2ccncc2[N+](=O)[O-])=C[C@@H](O[Si](C)(C)C(C)(C)C)[C@@H]1OS(=O)(=O)C(F)(F)F]

(2R,3R,4R)-4-(tert-butyldimethylsilyloxy)-6-(3-nitropyridin-4-yl)-2-vinyl-3,4-dihydro-2H-pyran-3-ol (1.0 equiv.) was dissolved in DCM (0.10 M) and cooled in an ice water bath. pyridine (4.0 equiv.) was added, followed by trifluoromethanesulfonic anhydride (2.0 equiv.) in a dropwise fashion and DMAP (0.2 equiv.) was added. The mixture was stirred for 2.5 h at 0° C. The reaction mixture was diluted with water and extracted with DCM. The combined organics were dried over sodium sulfate, filtered, and concentrated. The crude material was purified by flash chromatography over silica gel (heptanes:ethyl acetate gradient) to give (2R,3R,4R)-4-(tert-butyldimethylsilyloxy)-6-(3-nitropyridin-4-yl)-2-vinyl-3,4-dihydro-2H-pyran-3-yl trifluoromethanesulfonate in 57% yield.

### Synthesis of (2R,3R,4R,6R)-6-(3-aminopyridin-4-yl)-4-(tert-butyldimethylsilyloxy)-2-ethyltetrahydro-2H-pyran-3-yl trifluoromethanesulfonate

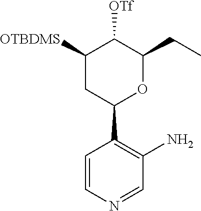 [SMILES: CC[C@H]1O[C@@H](c2ccncc2N)C[C@@H](O[Si](C)(C)C(C)(C)C)[C@@H]1OS(=O)(=O)C(F)(F)F]

(2R,3R,4R)-4-(tert-butyldimethylsilyloxy)-6-(3-nitropyridin-4-yl)-2-vinyl-3,4-dihydro-2H-pyran-3-yl trifluoromethanesulfonate (1.0 equiv.) was dissolved in EtOAc (0.04 M). Argon was bubbled through the mixture for 5 min. 10% palladium on carbon (0.25 equiv.) was added. The reaction vessel was evacuated and filled with hydrogen twice. The reaction was allowed to stir under a hydrogen balloon overnight. The reaction mixture was diluted with ethyl acetate and neutralized with saturated aqueous sodium bicarbonate. The mixture was filtered through Celite. The filtrate was concentrated. The residue was purified by flash chromatography over silica gel (heptanes:ethyl acetate gradient+1% triethylamine) to give (2R,3R,4R,6R)-6-(3-aminopyridin-4-yl)-4-(tert-butyldimethylsilyloxy)-2-ethyltetrahydro-2H-pyran-3-yl trifluoromethanesulfonate in 8% yield. LC/MS (m/z): 485.1 (MH⁺), Rₜ=1.09 min.

### Synthesis of (2R,3S,4R,6R)-6-(3-aminopyridin-4-yl)-4-(tert-butyldimethylsilyloxy)-2-ethyltetrahydro-2H-pyran-3-carbonitrile

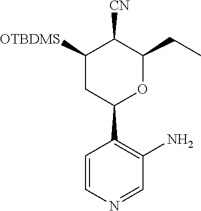 [SMILES: CC[C@H]1O[C@@H](c2ccncc2N)C[C@@H](O[Si](C)(C)C(C)(C)C)[C@H]1C#N]

(2R,3R,4R,6R)-6-(3-aminopyridin-4-yl)-4-(tert-butyldimethylsilyloxy)-2-ethyltetrahydro-2H-pyran-3-yl trifluoromethanesulfonate (1.0 equiv.) was dissolved in DMF (0.19 M). Sodium cyanide (5.0 equiv.) was added. The mixture was stirred at 80° C. for 90 min. The cooled reaction mixture was diluted with water and extracted with ethyl acetate. The combined organics were dried over sodium sulfate, filtered, and concentrated. The crude product was purified by flash chromatography over silica gel (heptanes:ethyl acetate gradient) to give (2R,3S,4R,6R)-6-(3-aminopyridin-4-yl)-4-(tert-butyldimethylsilyloxy)-2-ethyltetrahydro-2H-pyran-3-carbonitrile in 100% yield. LC/MS (m/z): 362.1 (MH⁺), Rₜ=0.41 min.

### Synthesis of (+/−)-2-(3-(6-(2,6-difluorophenyl)-5-fluoropicolinamido)pyridin-4-yl)-2-(trifluoromethyl)-3,4-dihydro-2H-pyran-4-yl acetate

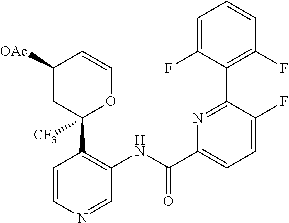 [SMILES: CC(=O)O[C@H]1C=CO[C@](c2ccncc2NC(=O)c2ccc(F)c(-c3c(F)cccc3F)n2)(C(F)(F)F)C1]

To a solution of 6-(2,6-difluorophenyl)-5-fluoropicolinic acid (1.0 equiv.) in DCM (0.2 M) was added 1-chloro-N,N,2-trimethylprop-1-en-1-amine (1.2 equiv.) and the reaction was stirred at room temperature for 30 min. To this solution was added to a solution of (+/−)-2-(3-aminopyridin-4-yl)-2-(trifluoromethyl)-3,4-dihydro-2H-pyran-4-yl acetate (1.0 equiv.) in THF (0.17 M) and pyridine (5 equiv.). The reaction turned light orange almost immediately. After 30 min, the reaction was quenched by the addition of saturated sodium bicarbonate and extracted with ethyl acetate. The organic phase was further washed with 1N NaOH, dried with sodium sulfate, filtered and concentrated to give (+/−)-2-(3-(6-(2,6-difluorophenyl)-5-fluoropicolinamido)pyridin-4-yl)-2-(trifluoromethyl)-3,4-dihydro-2H-pyran-4-yl acetate in 84% yield. The crude material was used for the next step without further purification. LC/MS (m/z): 538.3 (MH⁺) Rₜ=0.98 min.

### Synthesis of 6-(2,6-difluorophenyl)-5-fluoro-N-(4-((2R,4R)-4-hydroxy-2-(trifluoromethyl)-3,4-dihydro-2H-pyran-2-yl)pyridin-3-yl)picolinamide and 6-(2,6-difluorophenyl)-5-fluoro-N-((2S,4S)-4-hydroxy-2-(trifluoromethyl)-3,4-dihydro-2H-pyran-2-1 ride

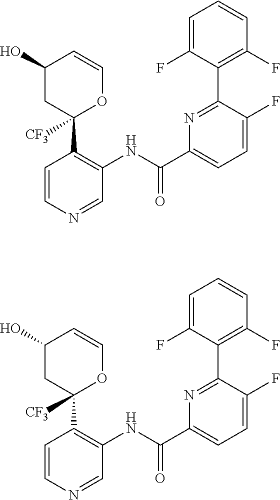 [SMILES: O=C(Nc1cnccc1[C@@]1(C(F)(F)F)C[C@@H](O)C=CO1)c1ccc(F)c(-c2c(F)cccc2F)n1 ; O=C(Nc1cnccc1[C@]1(C(F)(F)F)C[C@H](O)C=CO1)c1ccc(F)c(-c2c(F)cccc2F)n1]

To a solution of (+/−)-2-(3-(6-(2,6-difluorophenyl)-5-fluoropicolinamido)pyridin-4-yl)-2-(trifluoromethyl)-3,4-dihydro-2H-pyran-4-yl acetate (1.0 equiv.) in ethanol (0.05M) was added potassium carbonate (5 equiv.) and the reaction was stirred at 60° C. overnight. Upon cooling to room temperature, water was added and the volatiles were removed under vacuo. The crude was partitioned between ethyl acetate and water, the organic phase was dried with sodium sulfate and concentrated. The crude material was purified via silica gel column choromatography eluting with ethyl acetate and heptanes (0-50% ethyl acetate) to yield the desired product in 46% yield and 80% purity. This material was further purified via chiral HPLC eluting with heptane/ethanol (75/25, IC column) to give 6-(2,6-difluorophenyl)-5-fluoro-N-(4-((2R,4R)-4-hydroxy-2-(trifluoromethyl)-3,4-dihydro-2H-pyran-2-yl)pyridin-3-yl)picolinamide in 99% ee (LC/MS (m/z): 496.1 (MH⁺) Rₜ=0.97 min) and 6-(2,6-difluorophenyl)-5-fluoro-N-(4-((2S,4S)-4-hydroxy-2-(trifluoromethyl)-3,4-dihydro-2H-pyran-2-yl)pyridin-3-yl)picolinamide in 99% ee (LC/MS (m/z): 496.1 (MH⁺) Rₜ=0.97 min).

### Synthesis of (+/−)-2-(3-(6-(2,6-difluorophenyl)-5-fluoropicolinamido)pyridin-4-yl)-2-(trifluoromethyl)tetrahydro-2H-pyran-4-yl acetate and (+/−)-6-(2,6-difluorophenyl)-5-fluoro-N-(4-(2-(trifluoromethyl)tetrahydro-2H-pyran-2-yl)pyridin-3-yl)picolinamide

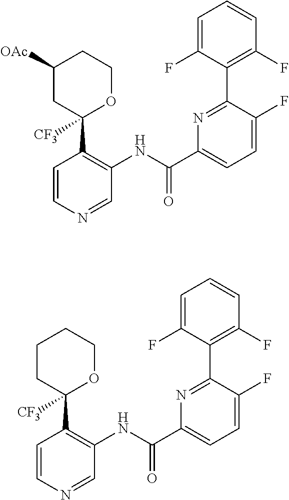 [SMILES: CC(=O)O[C@H]1CCO[C@](c2ccncc2NC(=O)c2ccc(F)c(-c3c(F)cccc3F)n2)(C(F)(F)F)C1 ; O=C(Nc1cnccc1[C@@]1(C(F)(F)F)CCCCO1)c1ccc(F)c(-c2c(F)cccc2F)n1]

To a solution of 6-(2,6-difluorophenyl)-5-fluoropicolinic acid (1.0 equiv.) in DCM (0.2 M) was added 1-chloro-N,N,2-trimethylprop-1-en-1-amine (1.2 equiv.) and the reaction was stirred at room temperature for 30 min. To this solution was added to a solution of (+/−)-2-(3-aminopyridin-4-yl)-2-(trifluoromethyl)tetrahydro-2H-pyran-4-yl acetate and (+/−)-4-(2-(trifluoromethyl)tetrahydro-2H-pyran-2-yl)pyridin-3-amine (1.0 equiv.) in THF (0.17 M) and pyridine (5 equiv.). The reaction turned light orange almost immediately. After 30 min, the reaction was quenched by the addition of saturated sodium carbonate and extracted with ethyl acetate. The organic phase was further washed with 1N NaOH, dried with sodium sulfate, filtered and concentrated to give (+/−)-2-(3-(6-(2,6-difluorophenyl)-5-fluoropicolinamido)pyridin-4-yl)-2-(trifluoromethyl)tetrahydro-2H-pyran-4-yl acetate and (+/−)-6-(2,6-difluorophenyl)-5-fluoro-N-(4-(2-(trifluoromethyl)tetrahydro-2H-pyran-2-yl)pyridin-3-yl)picolinamide in 90% yield as a mixture. The crude material was used for the next step without further purification. LC/MS (m/z): 540.3 (MH⁺) Rₜ=0.96 min and LC/MS (m/z): 482.2 (MH⁺) Rₜ=0.93 min.

### Synthesis of 6-(2,6-difluorophenyl)-5-fluoro-N-(4-((2R,4S)-4-hydroxy-2-(trifluoromethyl)tetrahydro-2H-pyran-2-yl)pyridin-3-yl)picolinamide, 6-(2,6-difluorophenyl)-5-fluoro-N-(4-((2S,4R)-4-hydroxy-2-(trifluoromethyl)tetrahydro-2H-pyran-2-yl)pyridin-3-yl)picolinamide, (S)-6-(2,6-difluorophenyl)-5-fluoro-N-(4-(2-(trifluoromethyl)tetrahydro-2H-pyran-2-yl)pyridin-3-yl)picolinamide and (R)-6-(2,6-difluorophenyl)-5-fluoro-N-(4-(2-(trifluoromethyl)tetrahydro-2H-pyran-2-yl)pyridin-3-yl)picolinamide

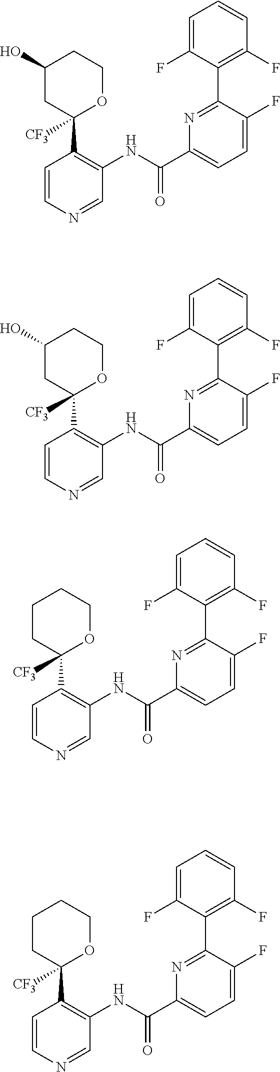 [SMILES: O=C(Nc1cnccc1[C@@]1(C(F)(F)F)C[C@@H](O)CCO1)c1ccc(F)c(-c2c(F)cccc2F)n1 ; O=C(Nc1cnccc1[C@]1(C(F)(F)F)C[C@H](O)CCO1)c1ccc(F)c(-c2c(F)cccc2F)n1 ; O=C(Nc1cnccc1[C@]1(C(F)(F)F)CCCCO1)c1ccc(F)c(-c2c(F)cccc2F)n1 ; O=C(Nc1cnccc1[C@@]1(C(F)(F)F)CCCCO1)c1ccc(F)c(-c2c(F)cccc2F)n1]

To a solution of (+/−)-2-(3-(6-(2,6-difluorophenyl)-5-fluoropicolinamido)pyridin-4-yl)-2-(trifluoromethyl)tetrahydro-2H-pyran-4-yl acetate and (+/−)-6-(2,6-difluorophenyl)-5-fluoro-N-(4-(2-(trifluoromethyl)tetrahydro-2H-pyran-2-yl)pyridin-3-yl)picolinamide (1.0 equiv.) in ethanol (0.05M) was added potassium carbonate (5 equiv.) and the reaction was stirred at 60° C. for 2 hours. Upon cooling to room temperature, water was added and the volatiles were removed under vacuo. The crude was partitioned between ethyl acetate and water, the organic phase was dried with sodium sulfate and concentrated. The crude material was purified via silica gel column chromatography eluting with ethyl acetate and heptanes (0-100% ethyl acetate) to yield 6-(2,6-difluorophenyl)-5-fluoro-N-(4-((+/−)-4-hydroxy-2-(trifluoromethyl)tetrahydro-2H-pyran-2-yl)pyridin-3-yl)picolinamide in 36% yield. The material was further purified via chiral HPLC eluting with heptane/ethanol (75/25, IC column) to give 6-(2,6-difluorophenyl)-5-fluoro-N-(4-((2R,4S)-4-hydroxy-2-(trifluoromethyl)tetrahydro-2H-pyran-2-yl)pyridin-3-yl)picolinamide (>99% ee) LC/MS (m/z): 498.3 (MH⁺) Rₜ=0.81 min and 6-(2,6-difluorophenyl)-5-fluoro-N-(4-((2S,4R)-4-hydroxy-2-(trifluoromethyl)tetrahydro-2H-pyran-2-yl)pyridin-3-yl)picolinamide (>99% ee) LC/MS (m/z): 498.3 (MH⁺) Rₜ=0.81 min. Compound (+/−)-6-(2,6-difluorophenyl)-5-fluoro-N-(4-(2-(trifluoromethyl)tetrahydro-2H-pyran-2-yl)pyridin-3-yl)picolinamide was also obtained in 25% yield. The material was further purified via chiral HPLC eluting with heptane/ethanol (80/20, IC column) to give (S)-6-(2,6-difluorophenyl)-5-fluoro-N-(4-(2-(trifluoromethyl)tetrahydro-2H-pyran-2-yl)pyridin-3-yl)picolinamide (>99% ee) LC/MS (m/z): 482.2 (MH⁺) Rₜ=0.92 min and (R)-6-(2,6-difluorophenyl)-5-fluoro-N-(4-(2-(trifluoromethyl)tetrahydro-2H-pyran-2-yl)pyridin-3-yl)picolinamide (>99% ee) LC/MS (m/z): 482.2 (MH⁺) Rₜ=0.92 min.

### Method 13

A homogeneous solution of 1 eq each of amine, carboxylic acid, HOAT and EDC in DMF, at a concentration of 0.5 M, was left standing for 24 hours at which time water and ethyl acetate were added. The organic phase was dried with sodium sulfate and purified via silica gel column chromatography eluting with ethyl acetate and hexanes to give the desired protected amide product. Alternatively the crude reaction mixture was directly purified by HPLC. Upon lyophilization, the TFA salt of the protected amide product was obtained. Alternatively, the HPLC fractions could be added to EtOAc and solid Na₂CO₃, separated and washed with NaCl_{(sat.)}. Upon drying over MgSO₄, filtering and removing the volatiles in vacuo, the protected amide product was obtained as a free base. Alternatively, the crude reaction mixture was used for the deprotection step without further purification.

If an N-Boc protected amine was present, it was removed by treating with excess 4M HCl/dioxane for 14 hours or by treating with 25% TFA/CH₂Cl₂ for 2 hours. Upon removal of the volatiles in vacuo, the material was purified by RP HPLC yielding after lyophilization the amide product as the TFA salt. Alternatively, the HPLC fractions could be added to EtOAc and solid Na₂CO₃, separated and washed with NaCl_{(sat.)}. Upon drying over MgSO₄, filtering and removing the volatiles in vacuo the free base was obtained. Upon dissolving in MeCN/H₂O, adding 1 eq. of 1 N HCl and lyophilizing, the HCl salt of the amide product was obtained.

If an OAc group was present, the acetate group could be cleaved by treating with K₂CO₃ (2.0 equiv.) in ethanol at a concentration of 0.1 M for 24 hours.

If a TBDMS or TIPS ether was present, it could be deprotected by treating with 6N HCl, THF, methanol (1:2:1) at room temperature or 60° C. for 12-24 h. Alternatively, the TBDMS or TIPS ether group could be deprotected by treating with tetrabutylammonium fluoride or tetramethylammoniumfluoride in THF at rt or 50-60° C.

If an OBn group was present, it was deprotected by treatment with 10% Pd/C (0.2 equiv.) under an atmosphere of hydrogen in ethyl acetate and methanol (1:2). Upon completion, the reaction was filtered through Celite, washed with methanol, and the filtrate was concentrated in vacuo.

The following compounds of the invention (Table 1) were prepared as described above or by means of METHOD 13. Table 1 lists compound structures, their molecular weights (both calculated and experimental), and retention times in minutes.

TABLE 1

| | | | LC/MS | LC/MS |
|---|---|---|---|---|
| Ex # | Structure | MW | (M + H) | (Rt) |
| 1 | 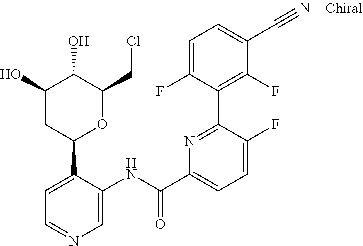 [SMILES: N#Cc1ccc(F)c(-c2nc(C(=O)Nc3cnccc3[C@H]3C[C@@H](O)[C@H](O)[C@@H](CCl)O3)ccc2F)c1F] | 518.9 | 519.0 | 0.65 |
| 2 | 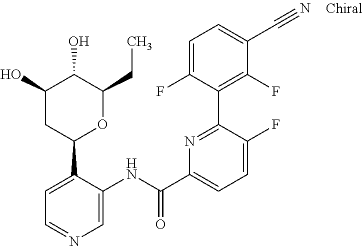 [SMILES: CC[C@H]1O[C@@H](c2ccncc2NC(=O)c2ccc(F)c(-c3c(F)ccc(C#N)c3F)n2)C[C@@H](O)[C@@H]1O] | 498.5 | 498.9 | 0.65 |
| 3 | 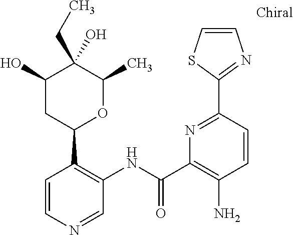 [SMILES: CC[C@]1(O)[C@H](O)C[C@H](c2ccncc2NC(=O)c2nc(-c3nccs3)ccc2N)O[C@@H]1C] | 455.5 | 456.1 | 0.53 |
| 4 | 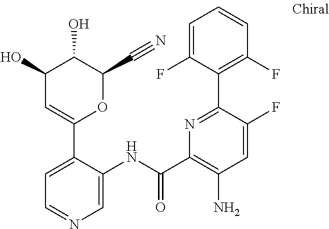 [SMILES: N#C[C@H]1OC(c2ccncc2NC(=O)c2nc(-c3c(F)cccc3F)c(F)cc2N)=C[C@@H](O)[C@@H]1O] | 483.4 | 483.9 | 0.63 |
| 5 | 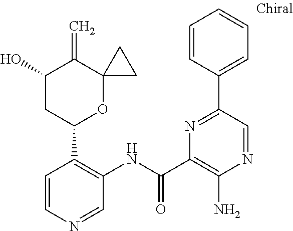 [SMILES: C=C1[C@@H](O)C[C@@H](c2ccncc2NC(=O)c2nc(-c3ccccc3)cnc2N)OC12CC2] | 429.5 | 430.1 | 0.70 |
| 6 | 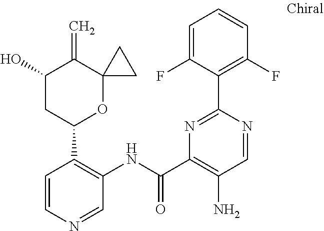 [SMILES: C=C1[C@@H](O)C[C@@H](c2ccncc2NC(=O)c2nc(-c3c(F)cccc3F)ncc2N)OC12CC2] | 465.5 | 466.1 | 0.64 |
| 7 | 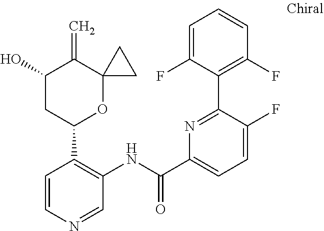 [SMILES: C=C1[C@@H](O)C[C@@H](c2ccncc2NC(=O)c2ccc(F)c(-c3c(F)cccc3F)n2)OC12CC2] | 467.4 | 468.1 | 0.75 |
| 8 | 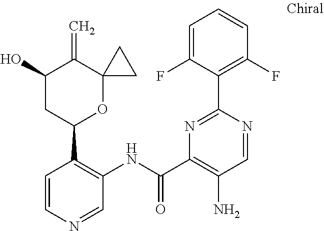 [SMILES: C=C1[C@H](O)C[C@H](c2ccncc2NC(=O)c2nc(-c3c(F)cccc3F)ncc2N)OC12CC2] | 465.5 | 466.1 | 0.64 |
| 9 | 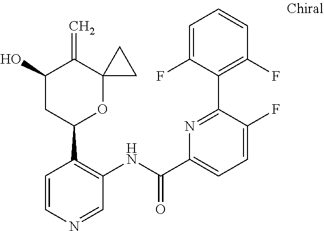 [SMILES: C=C1[C@H](O)C[C@H](c2ccncc2NC(=O)c2ccc(F)c(-c3c(F)cccc3F)n2)OC12CC2] | 467.4 | 468.1 | 0.75 |
| 10 | 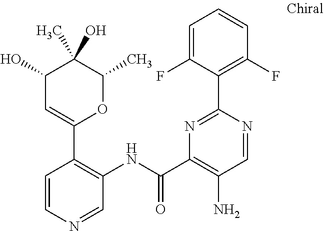 [SMILES: C[C@@H]1OC(c2ccncc2NC(=O)c2nc(-c3c(F)cccc3F)ncc2N)=C[C@H](O)[C@@]1(C)O] | 469.4 | 470.1 | 0.53 |
| 11 | 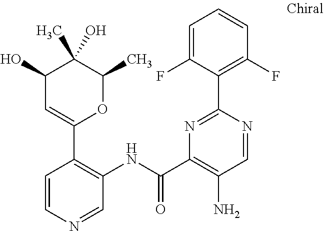 [SMILES: C[C@H]1OC(c2ccncc2NC(=O)c2nc(-c3c(F)cccc3F)ncc2N)=C[C@@H](O)[C@]1(C)O] | 469.4 | 470.1 | 0.53 |
| 12 | 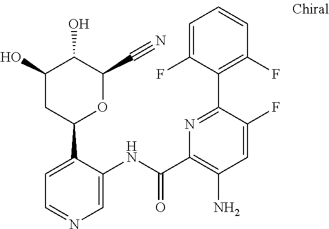 [SMILES: N#C[C@H]1O[C@@H](c2ccncc2NC(=O)c2nc(-c3c(F)cccc3F)c(F)cc2N)C[C@@H](O)[C@@H]1O] | 485.4 | 485.9 | 0.61 |
| 13 | 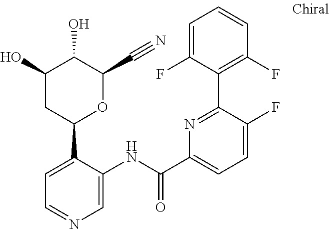 [SMILES: N#C[C@H]1O[C@@H](c2ccncc2NC(=O)c2ccc(F)c(-c3c(F)cccc3F)n2)C[C@@H](O)[C@@H]1O] | 470.4 | 470.9 | 0.61 |
| 14 | 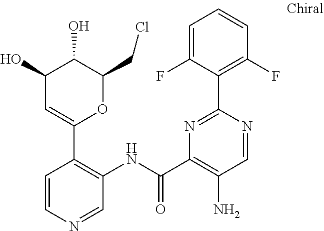 [SMILES: Nc1cnc(-c2c(F)cccc2F)nc1C(=O)Nc1cnccc1C1=C[C@@H](O)[C@H](O)[C@@H](CCl)O1] | 489.9 | 490.0 | 0.56 |
| 15 | 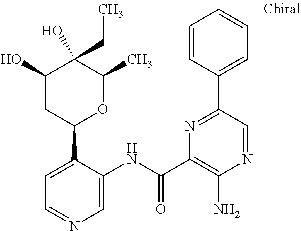 [SMILES: CC[C@]1(O)[C@H](O)C[C@H](c2ccncc2NC(=O)c2nc(-c3ccccc3)cnc2N)O[C@@H]1C] | 449.5 | 450.2 | 0.59 |
| 16 | 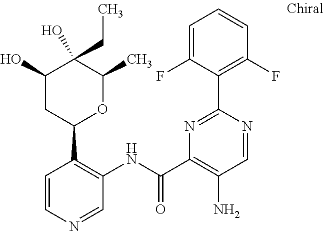 [SMILES: CC[C@]1(O)[C@H](O)C[C@H](c2ccncc2NC(=O)c2nc(-c3c(F)cccc3F)ncc2N)O[C@@H]1C] | 485.5 | 486.2 | 0.56 |
| 17 | 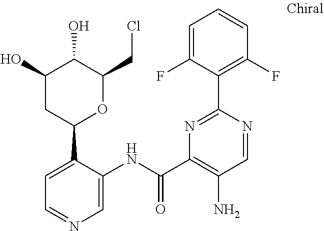 [SMILES: Nc1cnc(-c2c(F)cccc2F)nc1C(=O)Nc1cnccc1[C@H]1C[C@@H](O)[C@H](O)[C@@H](CCl)O1] | 491.9 | 492.1 | 0.54 |
| 18 | 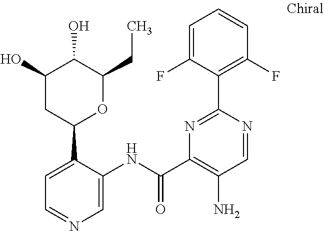 [SMILES: CC[C@H]1O[C@@H](c2ccncc2NC(=O)c2nc(-c3c(F)cccc3F)ncc2N)C[C@@H](O)[C@@H]1O] | 471.5 | 472.1 | 0.55 |
| 19 | 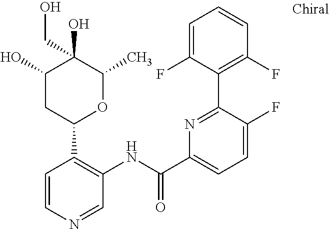 [SMILES: C[C@@H]1O[C@H](c2ccncc2NC(=O)c2ccc(F)c(-c3c(F)cccc3F)n2)C[C@H](O)[C@]1(O)CO] | 489.4 | 490.0 | 0.61 |
| 20 | 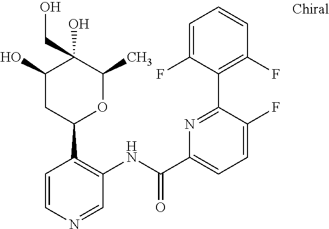 [SMILES: C[C@H]1O[C@@H](c2ccncc2NC(=O)c2ccc(F)c(-c3c(F)cccc3F)n2)C[C@@H](O)[C@@]1(O)CO] | 489.4 | 490.0 | 0.61 |
| 21 | 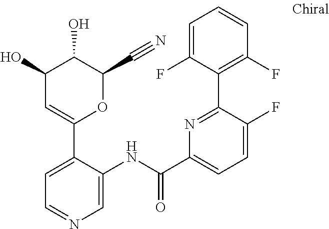 [SMILES: N#C[C@H]1OC(c2ccncc2NC(=O)c2ccc(F)c(-c3c(F)cccc3F)n2)=C[C@@H](O)[C@@H]1O] | 468.4 | 468.9 | 0.63 |
| 22 | 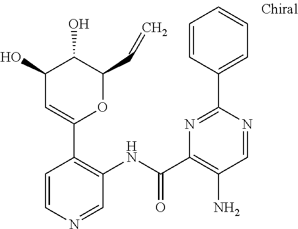 [SMILES: C=C[C@H]1OC(c2ccncc2NC(=O)c2nc(-c3ccccc3)ncc2N)=C[C@@H](O)[C@@H]1O] | 431.4 | 432.0 | 0.56 |
| 23 | 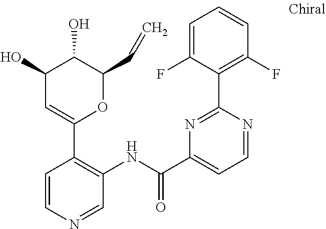 [SMILES: C=C[C@H]1OC(c2ccncc2NC(=O)c2ccnc(-c3c(F)cccc3F)n2)=C[C@@H](O)[C@@H]1O] | 452.4 | 453.0 | 0.55 |
| 24 | 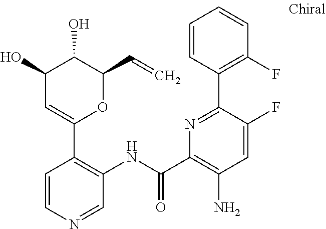 [SMILES: C=C[C@H]1OC(c2ccncc2NC(=O)c2nc(-c3ccccc3F)c(F)cc2N)=C[C@@H](O)[C@@H]1O] | 466.4 | 467.1 | 0.65 |
| 25 | 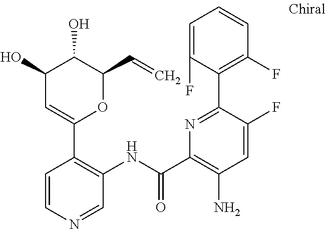 [SMILES: C=C[C@H]1OC(c2ccncc2NC(=O)c2nc(-c3c(F)cccc3F)c(F)cc2N)=C[C@@H](O)[C@@H]1O] | 484.4 | 485.1 | 0.65 |
| 26 | 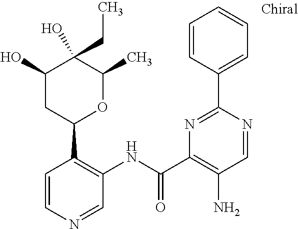 [SMILES: CC[C@]1(O)[C@H](O)C[C@H](c2ccncc2NC(=O)c2nc(-c3ccccc3)ncc2N)O[C@@H]1C] | 449.5 | 450.2 | 0.60 |
| 27 | 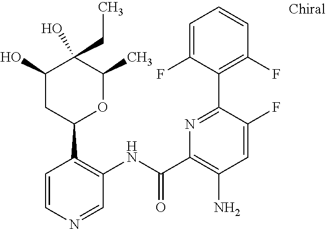 [SMILES: CC[C@]1(O)[C@H](O)C[C@H](c2ccncc2NC(=O)c2nc(-c3c(F)cccc3F)c(F)cc2N)O[C@@H]1C] | 502.5 | 503.0 | 0.66 |
| 28 | 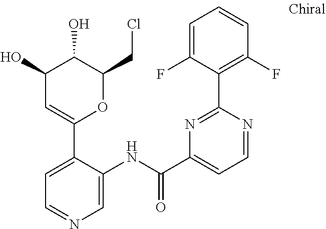 [SMILES: O=C(Nc1cnccc1C1=C[C@@H](O)[C@H](O)[C@@H](CCl)O1)c1ccnc(-c2c(F)cccc2F)n1] | 474.8 | 474.8 | 0.57 |
| 29 | 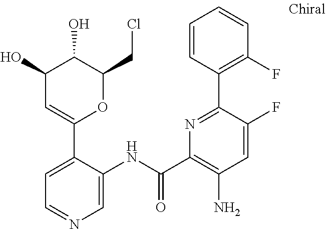 [SMILES: Nc1cc(F)c(-c2ccccc2F)nc1C(=O)Nc1cnccc1C1=C[C@@H](O)[C@H](O)[C@@H](CCl)O1] | 488.9 | 488.9 | 0.65 |
| 30 | 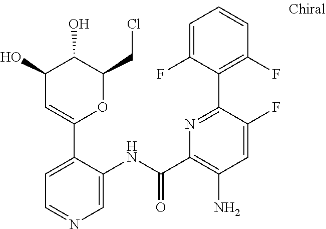 [SMILES: Nc1cc(F)c(-c2c(F)cccc2F)nc1C(=O)Nc1cnccc1C1=C[C@@H](O)[C@H](O)[C@@H](CCl)O1] | 506.9 | 506.8 | 0.66 |
| 31 | 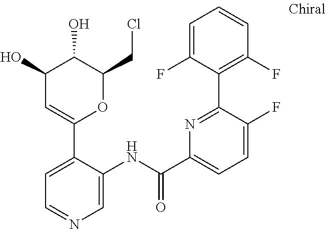 [SMILES: O=C(Nc1cnccc1C1=C[C@@H](O)[C@H](O)[C@@H](CCl)O1)c1ccc(F)c(-c2c(F)cccc2F)n1] | 491.9 | 491.9 | 0.66 |
| 32 | 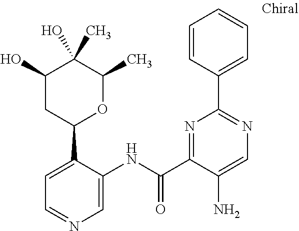 [SMILES: C[C@H]1O[C@@H](c2ccncc2NC(=O)c2nc(-c3ccccc3)ncc2N)C[C@@H](O)[C@]1(C)O] | 435.5 | 436.0 | 0.56 |
| 33 | 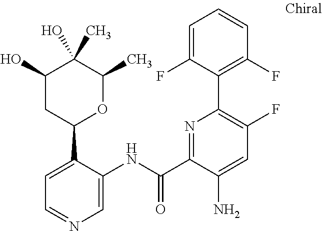 [SMILES: C[C@H]1O[C@@H](c2ccncc2NC(=O)c2nc(-c3c(F)cccc3F)c(F)cc2N)C[C@@H](O)[C@]1(C)O] | 488.5 | 489.0 | 0.64 |
| 34 | 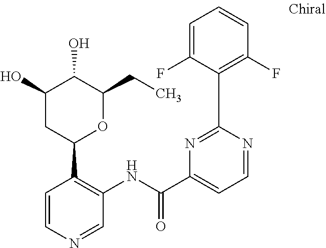 [SMILES: CC[C@H]1O[C@@H](c2ccncc2NC(=O)c2ccnc(-c3c(F)cccc3F)n2)C[C@@H](O)[C@@H]1O] | 456.4 | 457.1 | 0.58 |
| 35 | 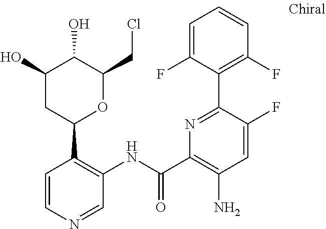 [SMILES: Nc1cc(F)c(-c2c(F)cccc2F)nc1C(=O)Nc1cnccc1[C@H]1C[C@@H](O)[C@H](O)[C@@H](CCl)O1] | 508.9 | 508.9 | 0.64 |
| 36 | 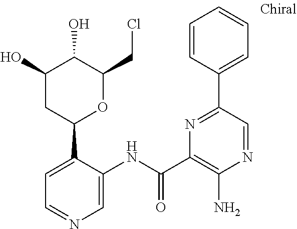 [SMILES: Nc1ncc(-c2ccccc2)nc1C(=O)Nc1cnccc1[C@H]1C[C@@H](O)[C@H](O)[C@@H](CCl)O1] | 455.9 | 456.0 | 0.57 |
| 37 | 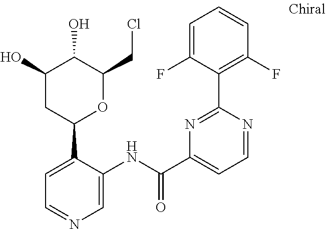 [SMILES: O=C(Nc1cnccc1[C@H]1C[C@@H](O)[C@H](O)[C@@H](CCl)O1)c1ccnc(-c2c(F)cccc2F)n1] | 476.9 | 477.0 | 0.55 |
| 38 | 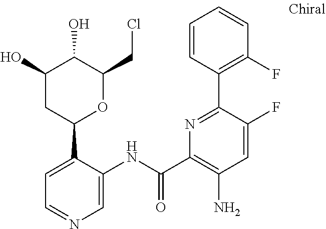 [SMILES: Nc1cc(F)c(-c2ccccc2F)nc1C(=O)Nc1cnccc1[C@H]1C[C@@H](O)[C@H](O)[C@@H](CCl)O1] | 490.9 | 491.0 | 0.64 |
| 39 | 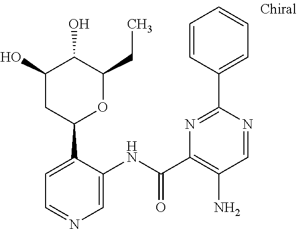 [SMILES: CC[C@H]1O[C@@H](c2ccncc2NC(=O)c2nc(-c3ccccc3)ncc2N)C[C@@H](O)[C@@H]1O] | 435.5 | 436.1 | 0.59 |
| 40 | 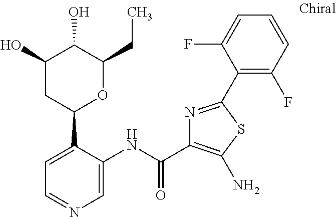 [SMILES: CC[C@H]1O[C@@H](c2ccncc2NC(=O)c2nc(-c3c(F)cccc3F)sc2N)C[C@@H](O)[C@@H]1O] | 476.5 | 477.1 | 0.63 |
| 41 | 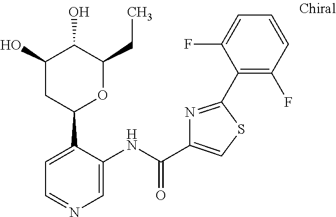 [SMILES: CC[C@H]1O[C@@H](c2ccncc2NC(=O)c2csc(-c3c(F)cccc3F)n2)C[C@@H](O)[C@@H]1O] | 461.5 | 462.1 | 0.64 |
| 42 | 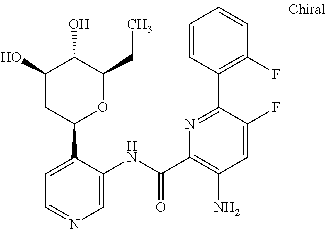 [SMILES: CC[C@H]1O[C@@H](c2ccncc2NC(=O)c2nc(-c3ccccc3F)c(F)cc2N)C[C@@H](O)[C@@H]1O] | 470.5 | 471.1 | 0.64 |
| 43 | 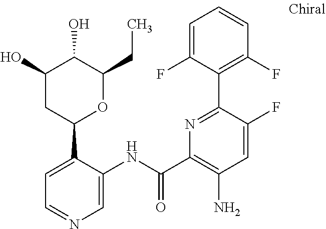 [SMILES: CC[C@H]1O[C@@H](c2ccncc2NC(=O)c2nc(-c3c(F)cccc3F)c(F)cc2N)C[C@@H](O)[C@@H]1O] | 488.5 | 489.2 | 0.66 |
| 44 | 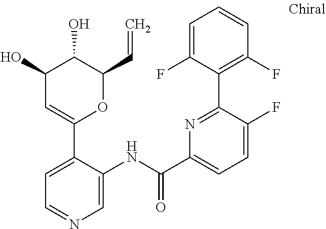 [SMILES: C=C[C@H]1OC(c2ccncc2NC(=O)c2ccc(F)c(-c3c(F)cccc3F)n2)=C[C@@H](O)[C@@H]1O] | 469.4 | 470.2 | 0.65 |
| 45 | 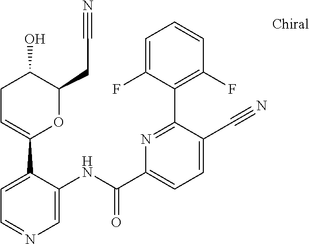 | 475.5 | 476.1 | 0.62 |
| 46 | 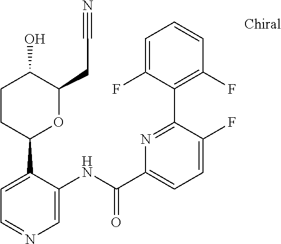 [SMILES: N#CC[C@H]1O[C@@H](c2ccncc2NC(=O)c2ccc(F)c(-c3c(F)cccc3F)n2)CC[C@@H]1O] | 468.4 | 469.1 | 0.66 |
| 47 | 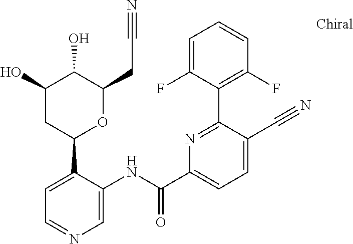 [SMILES: N#CC[C@H]1O[C@@H](c2ccncc2NC(=O)c2ccc(C#N)c(-c3c(F)cccc3F)n2)C[C@@H](O)[C@@H]1O] | 491.5 | 492.3 | 0.55 |
| 48 | 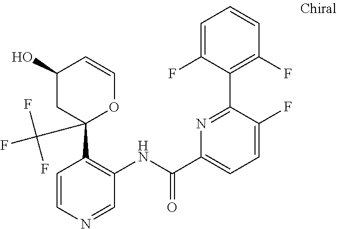 [SMILES: O=C(Nc1cnccc1[C@@]1(C(F)(F)F)C[C@@H](O)C=CO1)c1ccc(F)c(-c2c(F)cccc2F)n1] | 495.4 | 496.1 | 0.89 |
| 49 | 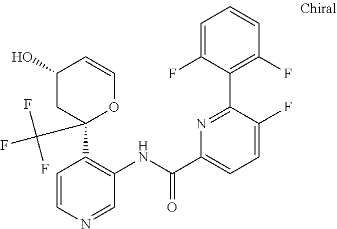 [SMILES: O=C(Nc1cnccc1[C@]1(C(F)(F)F)C[C@H](O)C=CO1)c1ccc(F)c(-c2c(F)cccc2F)n1] | 495.4 | 496.1 | 0.89 |
| 50 | 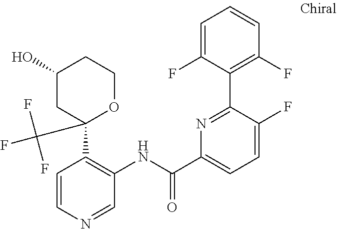 [SMILES: O=C(Nc1cnccc1[C@]1(C(F)(F)F)C[C@H](O)CCO1)c1ccc(F)c(-c2c(F)cccc2F)n1] | 497.4 | 498.2 | 0.81 |
| 51 | 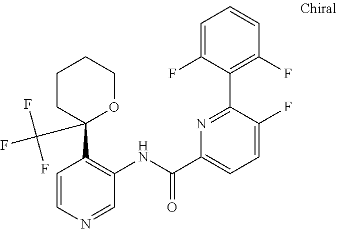 [SMILES: O=C(Nc1cnccc1[C@@]1(C(F)(F)F)CCCCO1)c1ccc(F)c(-c2c(F)cccc2F)n1] | 481.4 | 482.2 | 0.92 |
| 52 | 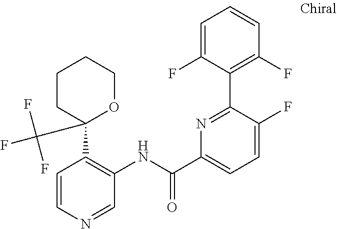 [SMILES: O=C(Nc1cnccc1[C@]1(C(F)(F)F)CCCCO1)c1ccc(F)c(-c2c(F)cccc2F)n1] | 481.4 | 482.2 | 0.92 |
| 53 | 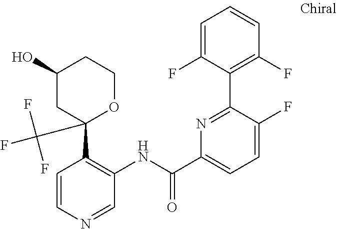 [SMILES: O=C(Nc1cnccc1[C@@]1(C(F)(F)F)C[C@@H](O)CCO1)c1ccc(F)c(-c2c(F)cccc2F)n1] | 497.4 | 498.2 | 0.81 |
| 54 | 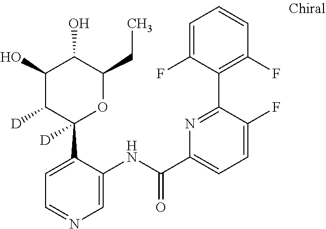 [SMILES: [2H][C@@H]1[C@@H](O)[C@H](O)[C@@H](CC)O[C@@]1([2H])c1ccncc1NC(=O)c1ccc(F)c(-c2c(F)cccc2F)n1] | 475.5 | 477.3 | 0.66 |
| 55 | 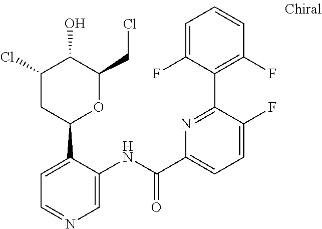 [SMILES: O=C(Nc1cnccc1[C@H]1C[C@H](Cl)[C@H](O)[C@@H](CCl)O1)c1ccc(F)c(-c2c(F)cccc2F)n1] | 512.3 | 512.2 | 0.74 |
| 56 | 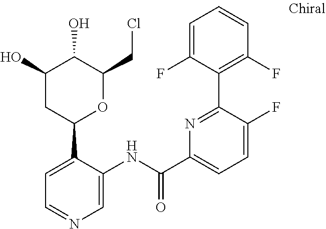 [SMILES: O=C(Nc1cnccc1[C@H]1C[C@@H](O)[C@H](O)[C@@H](CCl)O1)c1ccc(F)c(-c2c(F)cccc2F)n1] | 493.9 | 494.2 | 0.65 |
| 57 | 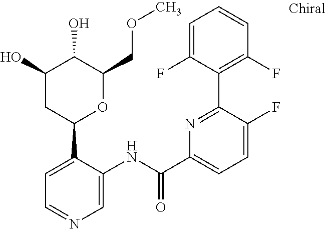 [SMILES: COC[C@H]1O[C@@H](c2ccncc2NC(=O)c2ccc(F)c(-c3c(F)cccc3F)n2)C[C@@H](O)[C@@H]1O] | 489.4 | 490.3 | 0.60 |
| 58 | 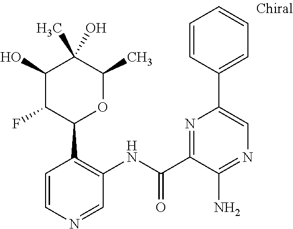 [SMILES: C[C@H]1O[C@@H](c2ccncc2NC(=O)c2nc(-c3ccccc3)cnc2N)[C@H](F)[C@@H](O)[C@]1(C)O] | 453.5 | 454.0 | 0.55 |
| 59 | 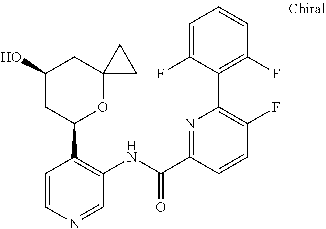 [SMILES: O=C(Nc1cnccc1[C@H]1C[C@@H](O)CC2(CC2)O1)c1ccc(F)c(-c2c(F)cccc2F)n1] | 455.4 | 456.0 | 0.70 |
| 60 | 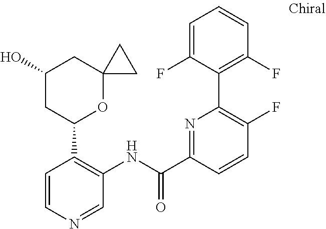 [SMILES: O=C(Nc1cnccc1[C@@H]1C[C@H](O)CC2(CC2)O1)c1ccc(F)c(-c2c(F)cccc2F)n1] | 455.4 | 456.0 | 0.70 |
| 61 | 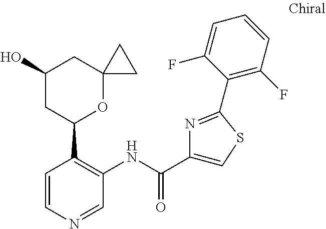 [SMILES: O=C(Nc1cnccc1[C@H]1C[C@@H](O)CC2(CC2)O1)c1csc(-c2c(F)cccc2F)n1] | 443.5 | 444.0 | 0.67 |
| 62 | 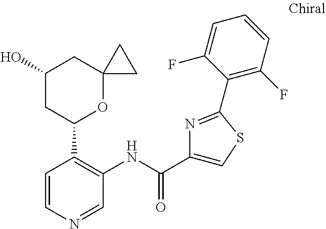 [SMILES: O=C(Nc1cnccc1[C@@H]1C[C@H](O)CC2(CC2)O1)c1csc(-c2c(F)cccc2F)n1] | 443.5 | 444.0 | 0.67 |
| 63 | 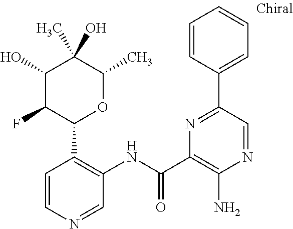 [SMILES: C[C@@H]1O[C@H](c2ccncc2NC(=O)c2nc(-c3ccccc3)cnc2N)[C@@H](F)[C@H](O)[C@@]1(C)O] | 453.5 | 454.3 | 0.56 |
| 64 | 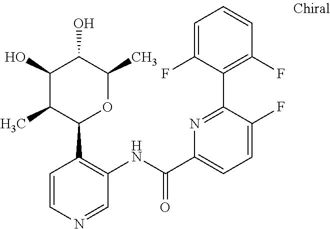 [SMILES: C[C@H]1[C@@H](O)[C@H](O)[C@@H](C)O[C@H]1c1ccncc1NC(=O)c1ccc(F)c(-c2c(F)cccc2F)n1] | 473.4 | 474.3 | 0.63 |
| 65 | 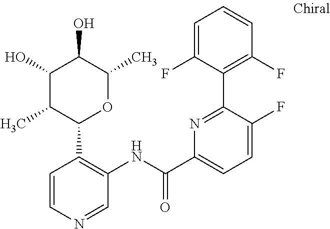 [SMILES: C[C@@H]1[C@H](O)[C@@H](O)[C@H](C)O[C@@H]1c1ccncc1NC(=O)c1ccc(F)c(-c2c(F)cccc2F)n1] | 473.4 | 474.3 | 0.63 |
| 66 | 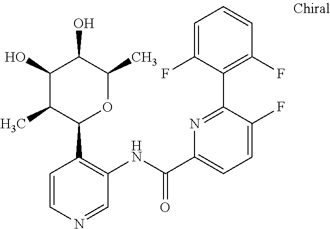 [SMILES: C[C@H]1[C@@H](O)[C@@H](O)[C@@H](C)O[C@H]1c1ccncc1NC(=O)c1ccc(F)c(-c2c(F)cccc2F)n1] | 473.4 | 474.2 | 0.63 |
| 67 | 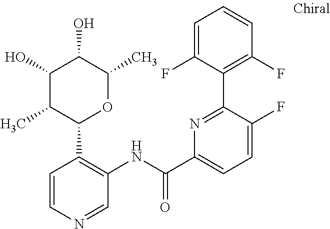 [SMILES: C[C@@H]1[C@H](O)[C@H](O)[C@H](C)O[C@@H]1c1ccncc1NC(=O)c1ccc(F)c(-c2c(F)cccc2F)n1] | 473.4 | 474.3 | 0.63 |
| 68 | 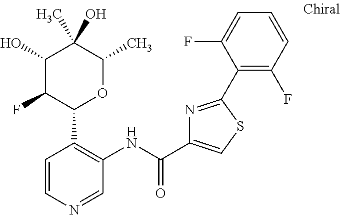 [SMILES: C[C@@H]1O[C@H](c2ccncc2NC(=O)c2csc(-c3c(F)cccc3F)n2)[C@@H](F)[C@H](O)[C@@]1(C)O] | 479.5 | 480.2 | 0.61 |
| 69 | 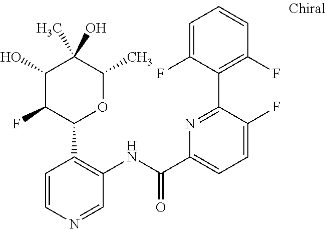 [SMILES: C[C@@H]1O[C@H](c2ccncc2NC(=O)c2ccc(F)c(-c3c(F)cccc3F)n2)[C@@H](F)[C@H](O)[C@@]1(C)O] | 491.4 | 492.3 | 0.63 |
| 70 | 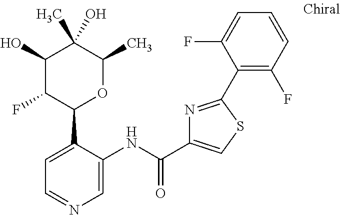 [SMILES: C[C@H]1O[C@@H](c2ccncc2NC(=O)c2csc(-c3c(F)cccc3F)n2)[C@H](F)[C@@H](O)[C@]1(C)O] | 479.5 | 480.0 | 0.61 |
| 71 | 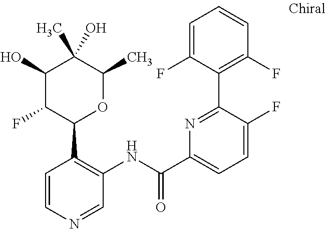 [SMILES: C[C@H]1O[C@@H](c2ccncc2NC(=O)c2ccc(F)c(-c3c(F)cccc3F)n2)[C@H](F)[C@@H](O)[C@]1(C)O] | 491.4 | 492.0 | 0.64 |
| 72 | 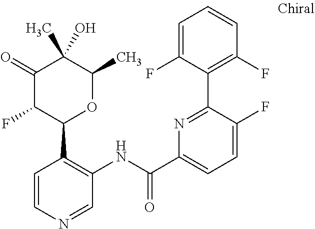 [SMILES: C[C@H]1O[C@@H](c2ccncc2NC(=O)c2ccc(F)c(-c3c(F)cccc3F)n2)[C@H](F)C(=O)[C@]1(C)O] | 489.4 | 490.3 | 0.64 |
| 73 | 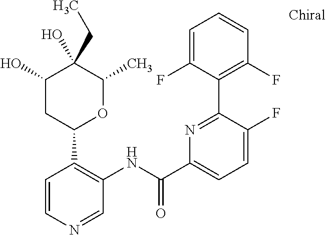 [SMILES: CC[C@@]1(O)[C@H](C)O[C@H](c2ccncc2NC(=O)c2ccc(F)c(-c3c(F)cccc3F)n2)C[C@@H]1O] | 487.5 | 488.3 | 0.71 |
| 74 | 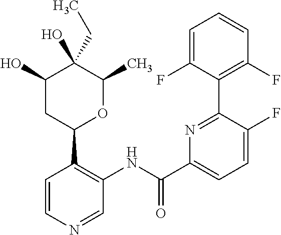 [SMILES: CC[C@@]1(O)[C@H](O)C[C@H](c2ccncc2NC(=O)c2ccc(F)c(-c3c(F)cccc3F)n2)O[C@@H]1C] | 487.5 | 488.3 | 0.71 |
| 75 | 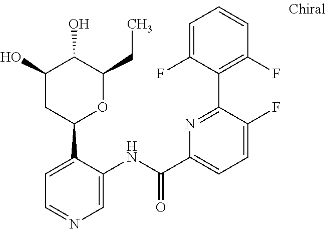 [SMILES: CC[C@H]1O[C@@H](c2ccncc2NC(=O)c2ccc(F)c(-c3c(F)cccc3F)n2)C[C@@H](O)[C@@H]1O] | 473.4 | 474.4 | 0.66 |
| 76 | 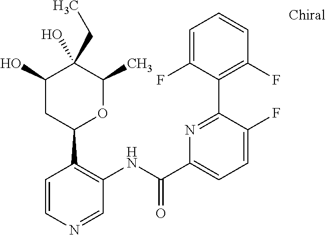 [SMILES: CC[C@]1(O)[C@H](O)C[C@H](c2ccncc2NC(=O)c2ccc(F)c(-c3c(F)cccc3F)n2)O[C@@H]1C] | 487.5 | 488.3 | 0.68 |
| 77 | 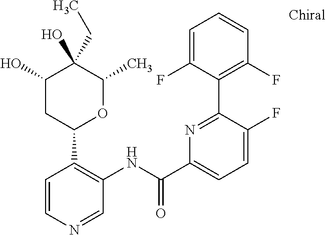 [SMILES: CC[C@]1(O)[C@H](C)O[C@H](c2ccncc2NC(=O)c2ccc(F)c(-c3c(F)cccc3F)n2)C[C@@H]1O] | 487.5 | 488.3 | 0.68 |
| 78 | 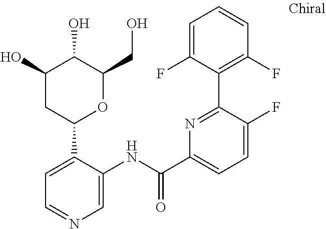 [SMILES: O=C(Nc1cnccc1[C@@H]1C[C@@H](O)[C@H](O)[C@@H](CO)O1)c1ccc(F)c(-c2c(F)cccc2F)n1] | 475.4 | 476.2 | 0.57 |
| 79 | 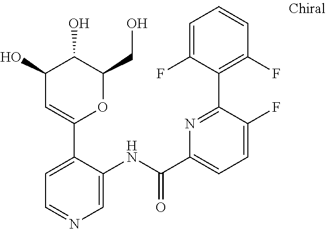 [SMILES: O=C(Nc1cnccc1C1=C[C@@H](O)[C@H](O)[C@@H](CO)O1)c1ccc(F)c(-c2c(F)cccc2F)n1] | 473.4 | 474.2 | 0.58 |
| 80 | 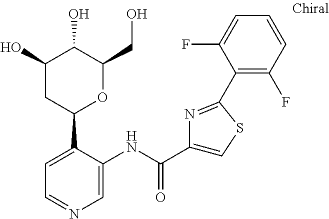 [SMILES: O=C(Nc1cnccc1[C@H]1C[C@@H](O)[C@H](O)[C@@H](CO)O1)c1csc(-c2c(F)cccc2F)n1] | 463.5 | 464.1 | 0.49 |
| 81 | 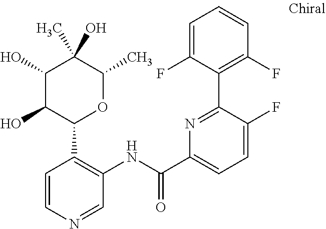 [SMILES: C[C@@H]1O[C@H](c2ccncc2NC(=O)c2ccc(F)c(-c3c(F)cccc3F)n2)[C@@H](O)[C@H](O)[C@@]1(C)O] | 489.4 | 490.3 | 0.61 |
| 82 | 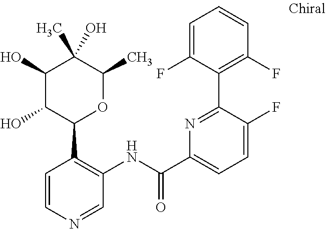 [SMILES: C[C@H]1O[C@@H](c2ccncc2NC(=O)c2ccc(F)c(-c3c(F)cccc3F)n2)[C@H](O)[C@@H](O)[C@]1(C)O] | 489.4 | 490.3 | 0.61 |
| 83 | 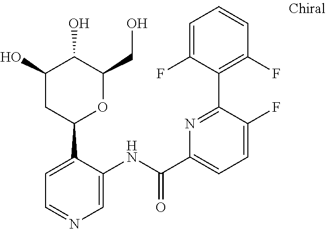 [SMILES: O=C(Nc1cnccc1[C@H]1C[C@@H](O)[C@H](O)[C@@H](CO)O1)c1ccc(F)c(-c2c(F)cccc2F)n1] | 475.4 | 476.2 | 0.53 |
| 84 | 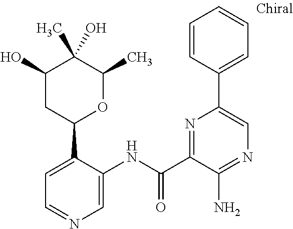 [SMILES: C[C@H]1O[C@@H](c2ccncc2NC(=O)c2nc(-c3ccccc3)cnc2N)C[C@@H](O)[C@]1(C)O] | 435.5 | 436.3 | 0.55 |
| 85 | 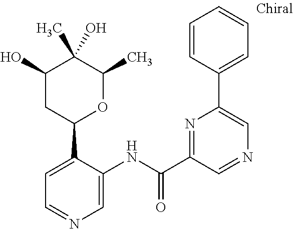 [SMILES: C[C@H]1O[C@@H](c2ccncc2NC(=O)c2cncc(-c3ccccc3)n2)C[C@@H](O)[C@]1(C)O] | 420.5 | 421.2 | 0.54 |
| 86 | 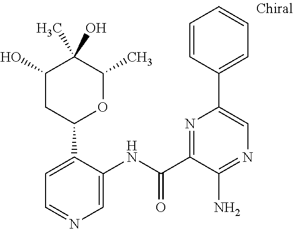 [SMILES: C[C@@H]1O[C@H](c2ccncc2NC(=O)c2nc(-c3ccccc3)cnc2N)C[C@H](O)[C@@]1(C)O] | 435.5 | 436.2 | 0.55 |
| 87 | 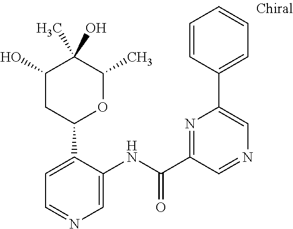 [SMILES: C[C@@H]1O[C@H](c2ccncc2NC(=O)c2cncc(-c3ccccc3)n2)C[C@H](O)[C@@]1(C)O] | 420.5 | 421.2 | 0.54 |
| 88 | 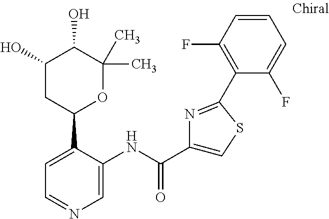 [SMILES: CC1(C)O[C@@H](c2ccncc2NC(=O)c2csc(-c3c(F)cccc3F)n2)C[C@H](O)[C@@H]1O] | 461.5 | 462.0 | 0.68 |
| 89 | 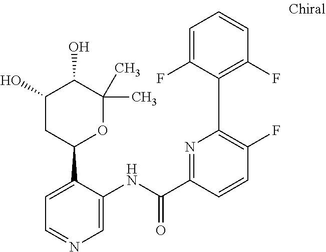 [SMILES: CC1(C)O[C@@H](c2ccncc2NC(=O)c2ccc(F)c(-c3c(F)cccc3F)n2)C[C@H](O)[C@@H]1O] | 473.4 | 474.0 | 0.69 |
| 90 | 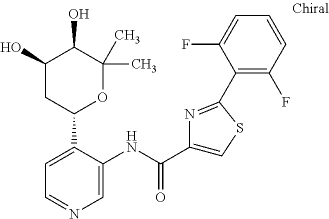 [SMILES: CC1(C)O[C@H](c2ccncc2NC(=O)c2csc(-c3c(F)cccc3F)n2)C[C@@H](O)[C@H]1O] | 461.5 | 462.0 | 0.66 |
| 91 | 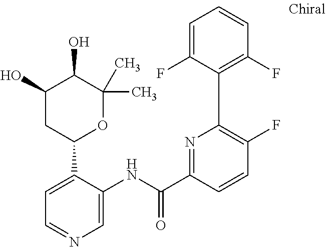 [SMILES: CC1(C)O[C@H](c2ccncc2NC(=O)c2ccc(F)c(-c3c(F)cccc3F)n2)C[C@@H](O)[C@H]1O] | 473.4 | 474.0 | 0.69 |
| 92 | 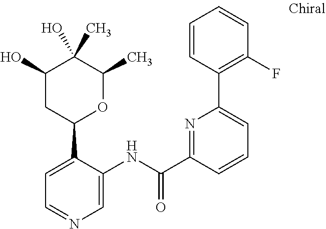 [SMILES: C[C@H]1O[C@@H](c2ccncc2NC(=O)c2cccc(-c3ccccc3F)n2)C[C@@H](O)[C@]1(C)O] | 437.5 | 438.0 | 0.63 |
| 93 | 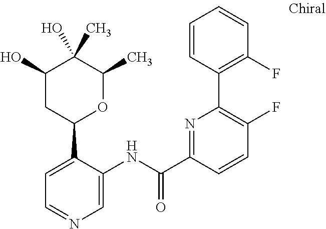 [SMILES: C[C@H]1O[C@@H](c2ccncc2NC(=O)c2ccc(F)c(-c3ccccc3F)n2)C[C@@H](O)[C@]1(C)O] | 455.5 | 456.0 | 0.65 |
| 94 | 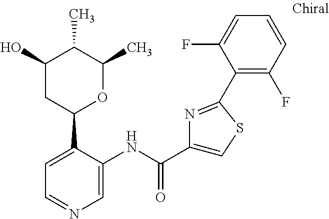 [SMILES: C[C@H]1[C@H](O)C[C@H](c2ccncc2NC(=O)c2csc(-c3c(F)cccc3F)n2)O[C@@H]1C] | 445.5 | 446.1 | 0.71 |
| 95 | 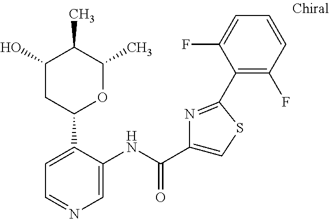 [SMILES: C[C@H]1[C@H](C)O[C@H](c2ccncc2NC(=O)c2csc(-c3c(F)cccc3F)n2)C[C@@H]1O] | 445.5 | 446.1 | 0.71 |
| 96 | 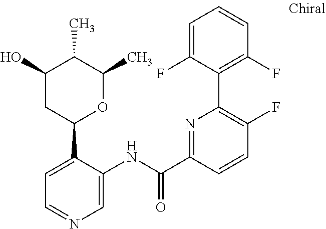 [SMILES: C[C@H]1[C@H](O)C[C@H](c2ccncc2NC(=O)c2ccc(F)c(-c3c(F)cccc3F)n2)O[C@@H]1C] | 457.4 | 458.1 | 0.73 |
| 97 | 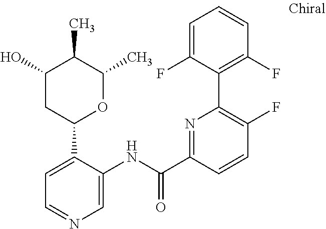 [SMILES: C[C@H]1[C@H](C)O[C@H](c2ccncc2NC(=O)c2ccc(F)c(-c3c(F)cccc3F)n2)C[C@@H]1O] | 457.4 | 458.1 | 0.73 |
| 98 | 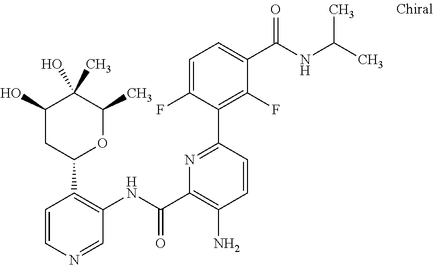 [SMILES: CC(C)NC(=O)c1ccc(F)c(-c2ccc(N)c(C(=O)Nc3cnccc3[C@@H]3C[C@@H](O)[C@](C)(O)[C@@H](C)O3)n2)c1F] | 555.6 | 556.3 | 0.60 |
| 99 | 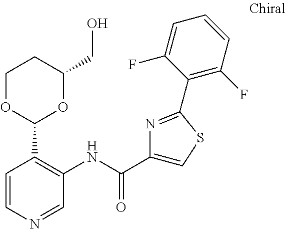 [SMILES: O=C(Nc1cnccc1[C@@H]1OCC[C@H](CO)O1)c1csc(-c2c(F)cccc2F)n1] | 433.4 | 434.0 | 0.64 |
| 100 | 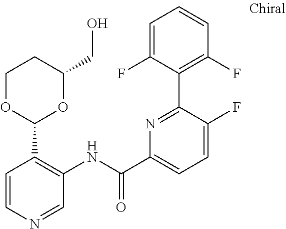 [SMILES: O=C(Nc1cnccc1[C@@H]1OCC[C@H](CO)O1)c1ccc(F)c(-c2c(F)cccc2F)n1] | 445.4 | 446.1 | 0.69 |
| 101 | 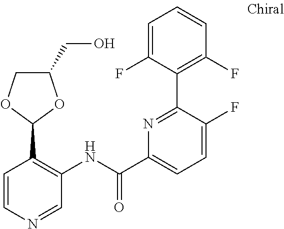 [SMILES: O=C(Nc1cnccc1[C@H]1OC[C@H](CO)O1)c1ccc(F)c(-c2c(F)cccc2F)n1] | 431.4 | 432.0 | 0.72 |
| 102 | 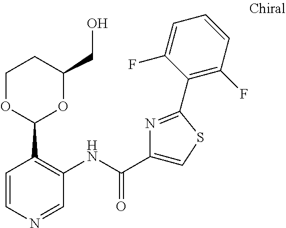 [SMILES: O=C(Nc1cnccc1[C@H]1OCC[C@@H](CO)O1)c1csc(-c2c(F)cccc2F)n1] | 433.4 | 434.0 | 0.63 |
| 103 | 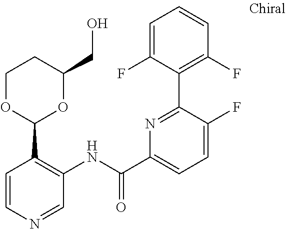 [SMILES: O=C(Nc1cnccc1[C@H]1OCC[C@@H](CO)O1)c1ccc(F)c(-c2c(F)cccc2F)n1] | 445.4 | 446.0 | 0.68 |
| 104 | 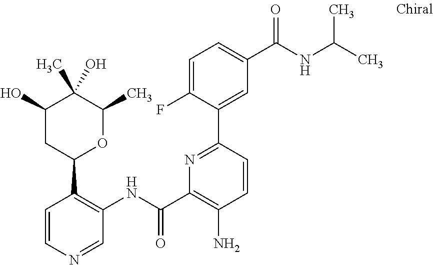 [SMILES: CC(C)NC(=O)c1ccc(F)c(-c2ccc(N)c(C(=O)Nc3cnccc3[C@H]3C[C@@H](O)[C@](C)(O)[C@@H](C)O3)n2)c1] | 537.6 | 538.2 | 0.58 |
| 105 | 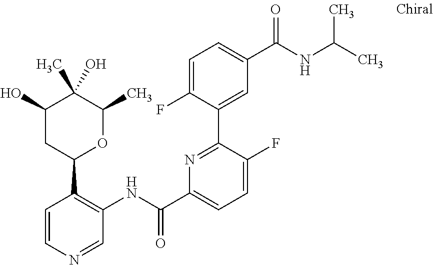 [SMILES: CC(C)NC(=O)c1ccc(F)c(-c2nc(C(=O)Nc3cnccc3[C@H]3C[C@@H](O)[C@](C)(O)[C@@H](C)O3)ccc2F)c1] | 540.6 | 541.1 | 0.60 |
| 106 | 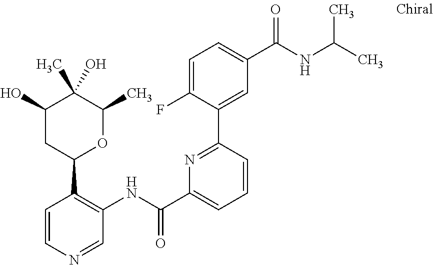 [SMILES: CC(C)NC(=O)c1ccc(F)c(-c2cccc(C(=O)Nc3cnccc3[C@H]3C[C@@H](O)[C@](C)(O)[C@@H](C)O3)n2)c1] | 522.6 | 523.1 | 0.59 |
| 107 | 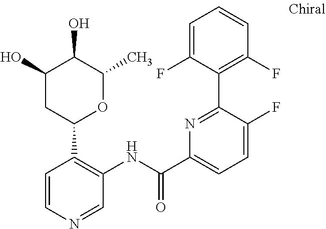 [SMILES: C[C@@H]1O[C@H](c2ccncc2NC(=O)c2ccc(F)c(-c3c(F)cccc3F)n2)C[C@@H](O)[C@H]1O] | 459.4 | 460.2 | 0.66 |
| 108 | 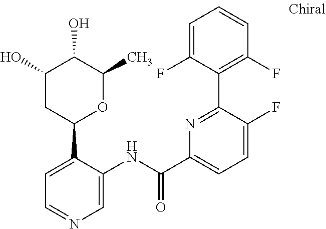 [SMILES: C[C@H]1O[C@@H](c2ccncc2NC(=O)c2ccc(F)c(-c3c(F)cccc3F)n2)C[C@H](O)[C@@H]1O] | 459.4 | 460.2 | 0.66 |
| 109 | 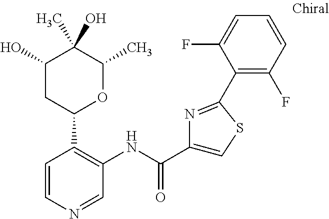 [SMILES: C[C@@H]1O[C@H](c2ccncc2NC(=O)c2csc(-c3c(F)cccc3F)n2)C[C@H](O)[C@@]1(C)O] | 461.5 | 462.0 | 0.60 |
| 110 | 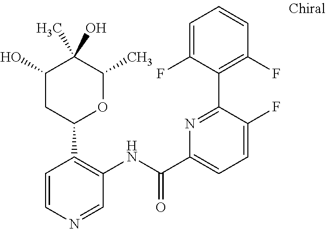 [SMILES: C[C@@H]1O[C@H](c2ccncc2NC(=O)c2ccc(F)c(-c3c(F)cccc3F)n2)C[C@H](O)[C@@]1(C)O] | 473.4 | 474.0 | 0.65 |
| 111 | 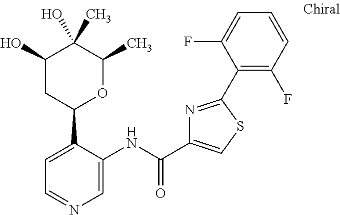 [SMILES: C[C@H]1O[C@@H](c2ccncc2NC(=O)c2csc(-c3c(F)cccc3F)n2)C[C@@H](O)[C@]1(C)O] | 461.5 | 462.1 | 0.60 |
| 112 | 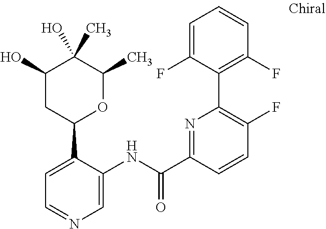 [SMILES: C[C@H]1O[C@@H](c2ccncc2NC(=O)c2ccc(F)c(-c3c(F)cccc3F)n2)C[C@@H](O)[C@]1(C)O] | 473.4 | 474.2 | 0.63 |
| 113 | 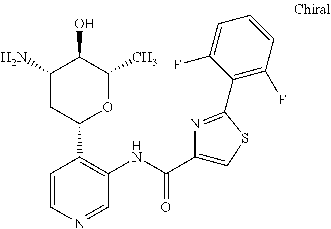 [SMILES: C[C@@H]1O[C@H](c2ccncc2NC(=O)c2csc(-c3c(F)cccc3F)n2)C[C@H](N)[C@H]1O] | 446.5 | 447.0 | 0.56 |
| 114 | 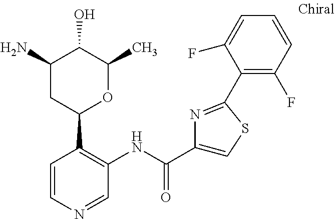 [SMILES: C[C@H]1O[C@@H](c2ccncc2NC(=O)c2csc(-c3c(F)cccc3F)n2)C[C@@H](N)[C@@H]1O] | 446.5 | 447.0 | 0.56 |
| 115 | 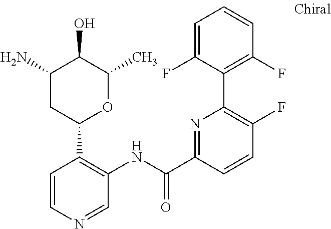 [SMILES: C[C@@H]1O[C@H](c2ccncc2NC(=O)c2ccc(F)c(-c3c(F)cccc3F)n2)C[C@H](N)[C@H]1O] | 458.4 | 459.0 | 0.59 |
| 116 | 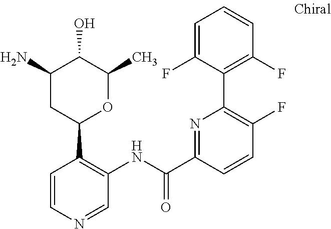 [SMILES: C[C@H]1O[C@@H](c2ccncc2NC(=O)c2ccc(F)c(-c3c(F)cccc3F)n2)C[C@@H](N)[C@@H]1O] | 458.4 | 459.0 | 0.59 |
| 117 | 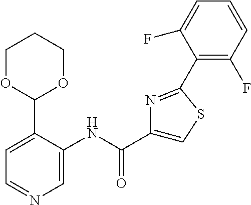 [SMILES: O=C(Nc1cnccc1C1OCCCO1)c1csc(-c2c(F)cccc2F)n1] | 403.4 | 404.1 | 72.28 |
| 118 | 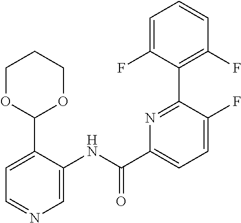 [SMILES: O=C(Nc1cnccc1C1OCCCO1)c1ccc(F)c(-c2c(F)cccc2F)n1] | 415.4 | 416.2 | 72.28 |
| 119 | 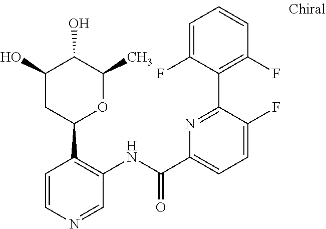 [SMILES: C[C@H]1O[C@@H](c2ccncc2NC(=O)c2ccc(F)c(-c3c(F)cccc3F)n2)C[C@@H](O)[C@@H]1O] | 459.4 | 460.1 | 0.62 |
| 120 | 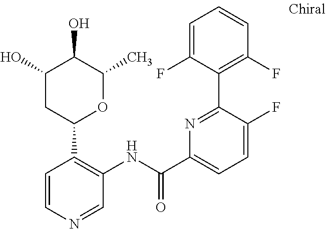 [SMILES: C[C@@H]1O[C@H](c2ccncc2NC(=O)c2ccc(F)c(-c3c(F)cccc3F)n2)C[C@H](O)[C@H]1O] | 459.4 | 460.1 | 0.62 |
| 121 | 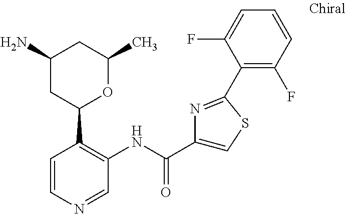 [SMILES: C[C@@H]1C[C@H](N)C[C@H](c2ccncc2NC(=O)c2csc(-c3c(F)cccc3F)n2)O1] | 430.5 | 431.1 | 0.57 |
| 122 | 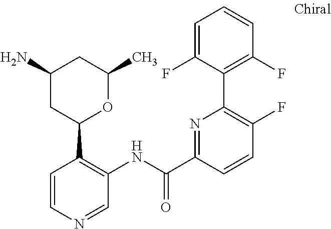 [SMILES: C[C@@H]1C[C@H](N)C[C@H](c2ccncc2NC(=O)c2ccc(F)c(-c3c(F)cccc3F)n2)O1] | 442.4 | 443.1 | 0.59 |
| 123 | 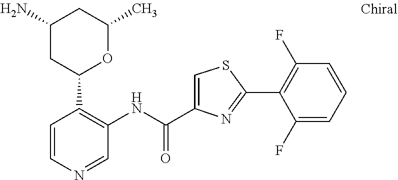 [SMILES: C[C@H]1C[C@@H](N)C[C@@H](c2ccncc2NC(=O)c2csc(-c3c(F)cccc3F)n2)O1] | 430.5 | 431.0 | 0.57 |
| 124 | 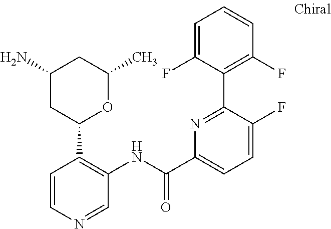 [SMILES: C[C@H]1C[C@@H](N)C[C@@H](c2ccncc2NC(=O)c2ccc(F)c(-c3c(F)cccc3F)n2)O1] | 442.4 | 443.0 | 0.60 |
| 125 | 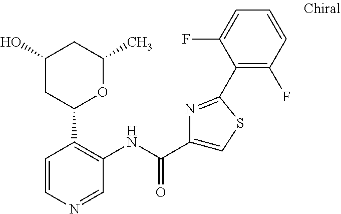 [SMILES: C[C@H]1C[C@@H](O)C[C@@H](c2ccncc2NC(=O)c2csc(-c3c(F)cccc3F)n2)O1] | 431.5 | 432.1 | 0.68 |
| 126 | 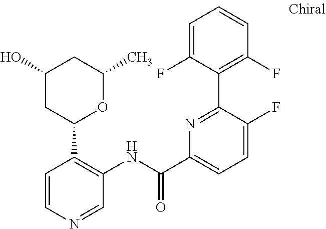 [SMILES: C[C@H]1C[C@@H](O)C[C@@H](c2ccncc2NC(=O)c2ccc(F)c(-c3c(F)cccc3F)n2)O1] | 443.4 | 444.1 | 0.70 |
| 127 | 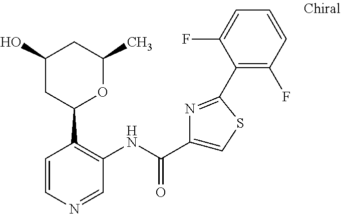 [SMILES: C[C@@H]1C[C@H](O)C[C@H](c2ccncc2NC(=O)c2csc(-c3c(F)cccc3F)n2)O1] | 431.5 | 432.1 | 0.67 |
| 128 | 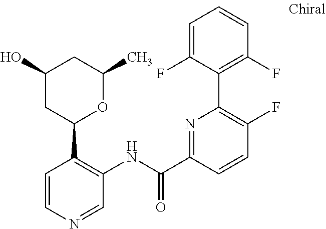 [SMILES: C[C@@H]1C[C@H](O)C[C@H](c2ccncc2NC(=O)c2ccc(F)c(-c3c(F)cccc3F)n2)O1] | 443.4 | 444.1 | 0.70 |
| 129 | 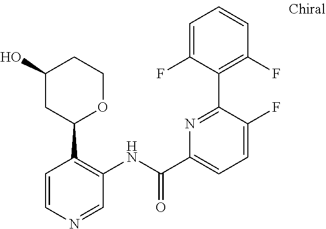 [SMILES: O=C(Nc1cnccc1[C@H]1C[C@@H](O)CCO1)c1ccc(F)c(-c2c(F)cccc2F)n1] | 429.4 | 430.1 | 0.68 |
| 130 | 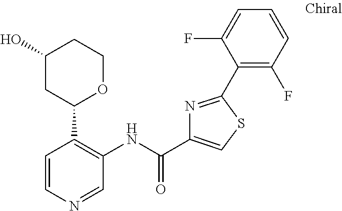 [SMILES: O=C(Nc1cnccc1[C@@H]1C[C@H](O)CCO1)c1csc(-c2c(F)cccc2F)n1] | 417.4 | 418.0 | 0.65 |
| 131 | 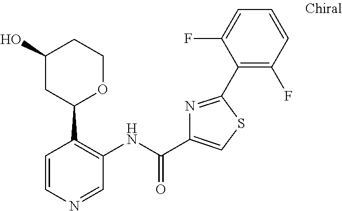 [SMILES: O=C(Nc1cnccc1[C@H]1C[C@@H](O)CCO1)c1csc(-c2c(F)cccc2F)n1] | 417.4 | 418.0 | 0.65 |
| 132 | 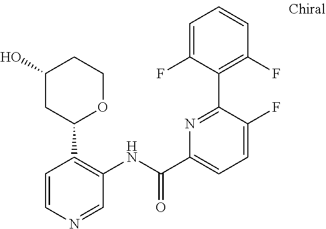 [SMILES: O=C(Nc1cnccc1[C@@H]1C[C@H](O)CCO1)c1ccc(F)c(-c2c(F)cccc2F)n1] | 429.4 | 430.1 | 0.68 |
| 133 | 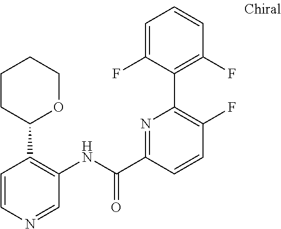 [SMILES: O=C(Nc1cnccc1[C@@H]1CCCCO1)c1ccc(F)c(-c2c(F)cccc2F)n1] | 413.4 | 414.0 | 0.78 |
| 134 | 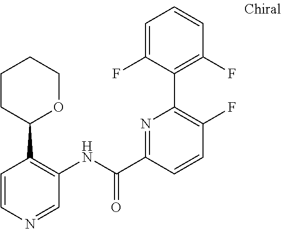 [SMILES: O=C(Nc1cnccc1[C@H]1CCCCO1)c1ccc(F)c(-c2c(F)cccc2F)n1] | 413.4 | 414.0 | 0.78 |
| 135 | 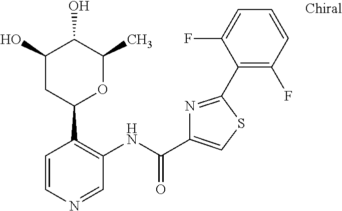 [SMILES: C[C@H]1O[C@@H](c2ccncc2NC(=O)c2csc(-c3c(F)cccc3F)n2)C[C@@H](O)[C@@H]1O] | 447.5 | 448.1 | 0.61 |
| 136 | 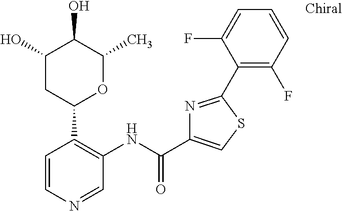 [SMILES: C[C@@H]1O[C@H](c2ccncc2NC(=O)c2csc(-c3c(F)cccc3F)n2)C[C@H](O)[C@H]1O] | 447.5 | 448.1 | 0.61 |
| 137 | 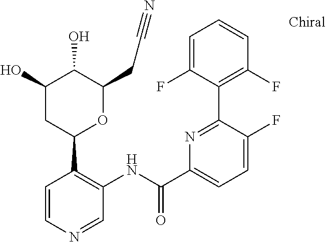 [SMILES: N#CC[C@H]1O[C@@H](c2ccncc2NC(=O)c2ccc(F)c(-c3c(F)cccc3F)n2)C[C@@H](O)[C@@H]1O] | 484.4 | 485.0 | 0.61 |
| 138 | 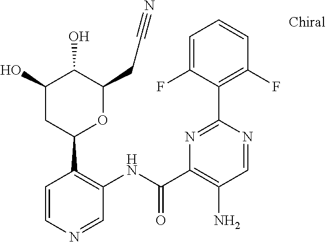 [SMILES: N#CC[C@H]1O[C@@H](c2ccncc2NC(=O)c2nc(-c3c(F)cccc3F)ncc2N)C[C@@H](O)[C@@H]1O] | 482.4 | 483.1 | 0.49 |
| 139 | 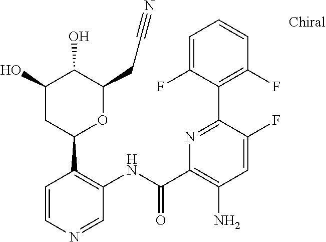 [SMILES: N#CC[C@H]1O[C@@H](c2ccncc2NC(=O)c2nc(-c3c(F)cccc3F)c(F)cc2N)C[C@@H](O)[C@@H]1O] | 499.4 | 500.0 | 0.61 |
| 140 | 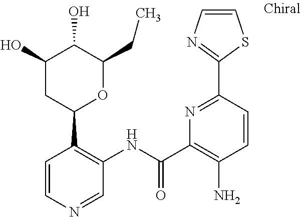 [SMILES: CC[C@H]1O[C@@H](c2ccncc2NC(=O)c2nc(-c3nccs3)ccc2N)C[C@@H](O)[C@@H]1O] | 441.5 | 442.0 | 0.51 |
| 141 | 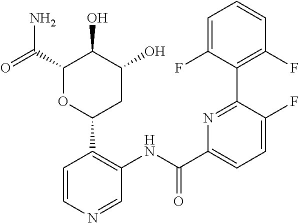 [SMILES: NC(=O)[C@H]1O[C@@H](c2ccncc2NC(=O)c2ccc(F)c(-c3c(F)cccc3F)n2)C[C@@H](O)[C@@H]1O] | 488.4 | 489.1 | 0.51 |
| 142 | 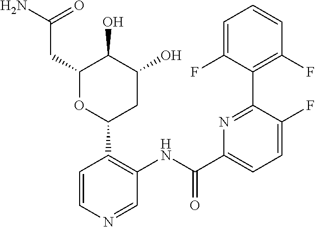 [SMILES: NC(=O)C[C@H]1O[C@@H](c2ccncc2NC(=O)c2ccc(F)c(-c3c(F)cccc3F)n2)C[C@@H](O)[C@@H]1O] | 502.4 | 503.1 | 0.51 |
| 143 | 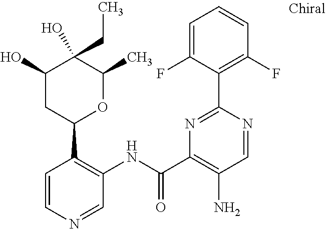 [SMILES: CC[C@]1(O)[C@H](O)C[C@H](c2ccncc2NC(=O)c2nc(-c3c(F)cccc3F)ncc2N)O[C@@H]1C] | 485.5 | 486.2 | 0.56 |
| 144 | 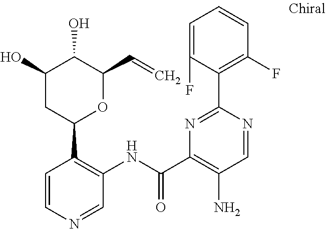 [SMILES: C=C[C@H]1O[C@@H](c2ccncc2NC(=O)c2nc(-c3c(F)cccc3F)ncc2N)C[C@@H](O)[C@@H]1O] | 467.4 | 468.0 | 0.54 |
| 145 | 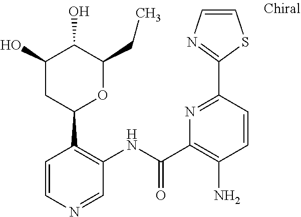 [SMILES: CC[C@H]1O[C@@H](c2ccncc2NC(=O)c2nc(-c3nccs3)ccc2N)C[C@@H](O)[C@@H]1O] | 441.5 | 442.0 | 0.51 |
| 146 | 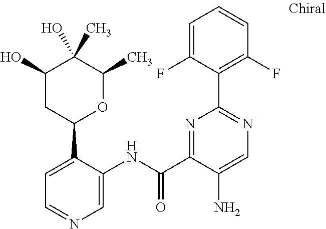 [SMILES: C[C@H]1O[C@@H](c2ccncc2NC(=O)c2nc(-c3c(F)cccc3F)ncc2N)C[C@@H](O)[C@]1(C)O] | 471.5 | 472.1 | 0.51 |
| 147 | 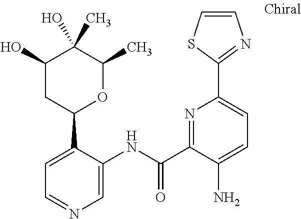 [SMILES: C[C@H]1O[C@@H](c2ccncc2NC(=O)c2nc(-c3nccs3)ccc2N)C[C@@H](O)[C@]1(C)O] | 441.5 | 442.1 | 0.47 |
| 148 | 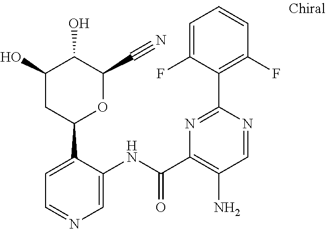 [SMILES: N#C[C@H]1O[C@@H](c2ccncc2NC(=O)c2nc(-c3c(F)cccc3F)ncc2N)C[C@@H](O)[C@@H]1O] | 468.4 | 469.1 | 0.51 |
| 149 | 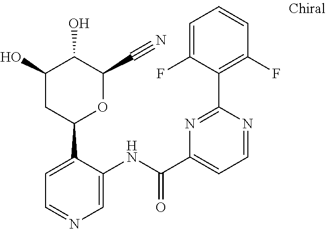 [SMILES: N#C[C@H]1O[C@@H](c2ccncc2NC(=O)c2ccnc(-c3c(F)cccc3F)n2)C[C@@H](O)[C@@H]1O] | 453.1 | 454.1 | 0.51 |
| 150 | 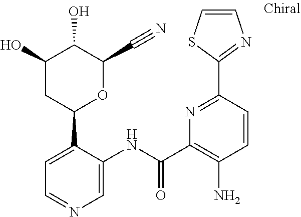 [SMILES: N#C[C@H]1O[C@@H](c2ccncc2NC(=O)c2nc(-c3nccs3)ccc2N)C[C@@H](O)[C@@H]1O] | 438.5 | 439.1 | 0.49 |
| 151 | 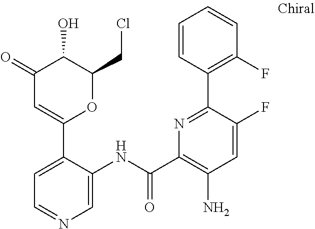 [SMILES: Nc1cc(F)c(-c2ccccc2F)nc1C(=O)Nc1cnccc1C1=CC(=O)[C@H](O)[C@@H](CCl)O1] | 486.9 | 487.0 | 0.77 |
| 152 | 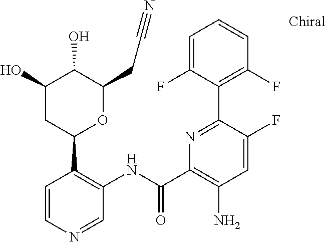 [SMILES: N#CC[C@H]1O[C@@H](c2ccncc2NC(=O)c2nc(-c3c(F)cccc3F)c(F)cc2N)C[C@@H](O)[C@@H]1O] | 499.4 | 500.0 | 0.61 |
| 153 | 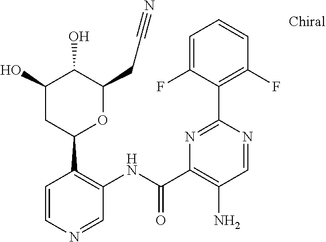 [SMILES: N#CC[C@H]1O[C@@H](c2ccncc2NC(=O)c2nc(-c3c(F)cccc3F)ncc2N)C[C@@H](O)[C@@H]1O] | 482.4 | 483.1 | 0.49 |
| 155 | 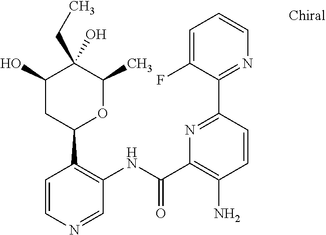 [SMILES: CC[C@]1(O)[C@H](O)C[C@H](c2ccncc2NC(=O)c2nc(-c3ncccc3F)ccc2N)O[C@@H]1C] | 467.5 | 468.1 | 0.49 |
| 156 | 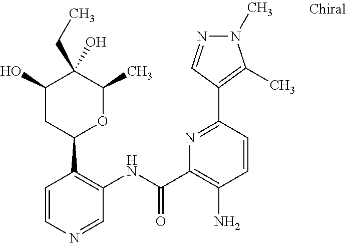 [SMILES: CC[C@]1(O)[C@H](O)C[C@H](c2ccncc2NC(=O)c2nc(-c3cnn(C)c3C)ccc2N)O[C@@H]1C] | 466.5 | 467.2 | 0.48 |
| 157 | 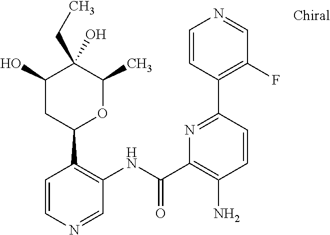 [SMILES: CC[C@]1(O)[C@H](O)C[C@H](c2ccncc2NC(=O)c2nc(-c3ccncc3F)ccc2N)O[C@@H]1C] | 467.5 | 468.1 | 0.45 |
| 158 | 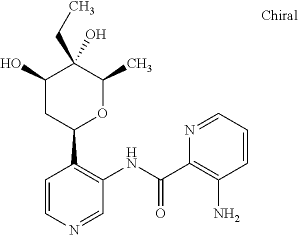 [SMILES: CC[C@]1(O)[C@H](O)C[C@H](c2ccncc2NC(=O)c2ncccc2N)O[C@@H]1C] | 372.4 | 373.1 | 0.49 |
| 159 | 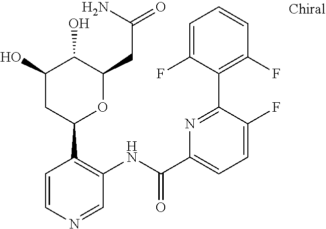 [SMILES: NC(=O)C[C@H]1O[C@@H](c2ccncc2NC(=O)c2ccc(F)c(-c3c(F)cccc3F)n2)C[C@@H](O)[C@@H]1O] | 502.4 | 503.1 | 0.52 |
| 160 | 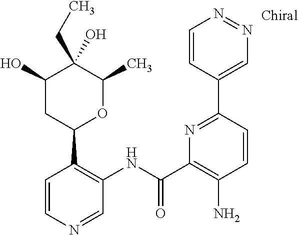 [SMILES: CC[C@]1(O)[C@H](O)C[C@H](c2ccncc2NC(=O)c2nc(-c3ccnnc3)ccc2N)O[C@@H]1C] | 450.5 | 451.1 | 0.39 |
| 161 | 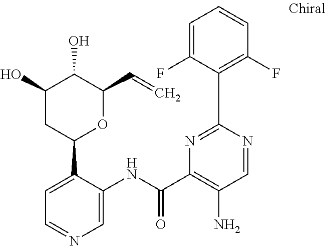 [SMILES: C=C[C@H]1O[C@@H](c2ccncc2NC(=O)c2nc(-c3c(F)cccc3F)ncc2N)C[C@@H](O)[C@@H]1O] | 469.4 | 470.1 | 0.52 |
| 162 | 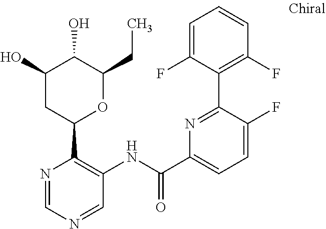 [SMILES: CC[C@H]1O[C@@H](c2ncncc2NC(=O)c2ccc(F)c(-c3c(F)cccc3F)n2)C[C@@H](O)[C@@H]1O] | 474.4 | 475.1 | 0.80 |
| 163 | 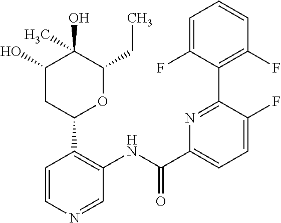 [SMILES: CC[C@@H]1O[C@H](c2ccncc2NC(=O)c2ccc(F)c(-c3c(F)cccc3F)n2)C[C@H](O)[C@@]1(C)O] | 487.5 | 488.1 | 0.67 |
| 164 | 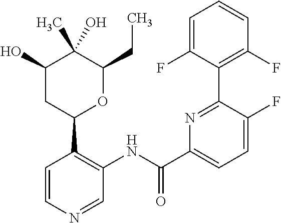 [SMILES: CC[C@H]1O[C@@H](c2ccncc2NC(=O)c2ccc(F)c(-c3c(F)cccc3F)n2)C[C@@H](O)[C@]1(C)O] | 487.5 | 488.1 | 0.66 |
| 165 | 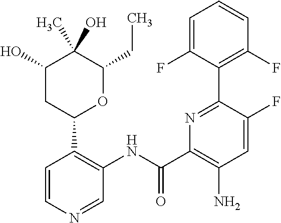 [SMILES: CC[C@@H]1O[C@H](c2ccncc2NC(=O)c2nc(-c3c(F)cccc3F)c(F)cc2N)C[C@H](O)[C@@]1(C)O] | 502.5 | 503.1 | 0.67 |
| 166 | 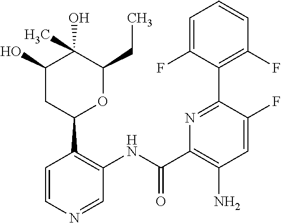 [SMILES: CC[C@H]1O[C@@H](c2ccncc2NC(=O)c2nc(-c3c(F)cccc3F)c(F)cc2N)C[C@@H](O)[C@]1(C)O] | 502.5 | 503.1 | 0.67 |
| 167 | 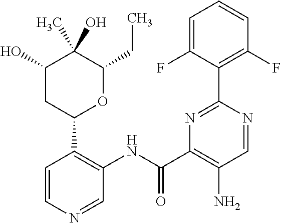 [SMILES: CC[C@@H]1O[C@H](c2ccncc2NC(=O)c2nc(-c3c(F)cccc3F)ncc2N)C[C@H](O)[C@@]1(C)O] | 485.5 | 486.1 | 0.56 |
| 168 | 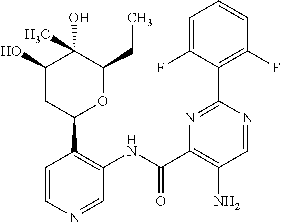 [SMILES: CC[C@H]1O[C@@H](c2ccncc2NC(=O)c2nc(-c3c(F)cccc3F)ncc2N)C[C@@H](O)[C@]1(C)O] | 485.5 | 486.1 | 0.56 |
| 169 | 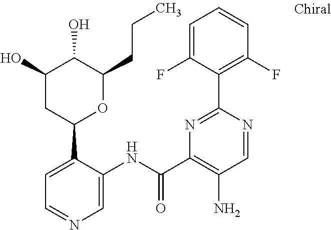 [SMILES: CCC[C@H]1O[C@@H](c2ccncc2NC(=O)c2nc(-c3c(F)cccc3F)ncc2N)C[C@@H](O)[C@@H]1O] | 485.5 | 486.1 | 0.60 |
| 170 | 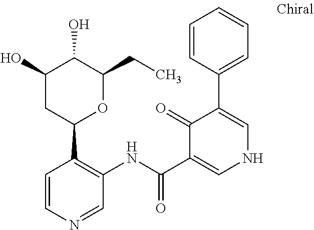 [SMILES: CC[C@H]1O[C@@H](c2ccncc2NC(=O)c2c[nH]cc(-c3ccccc3)c2=O)C[C@@H](O)[C@@H]1O] | 435.5 | 436.1 | 0.51 |
| 171 | 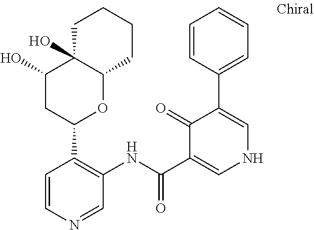 [SMILES: O=C(Nc1cnccc1[C@@H]1C[C@H](O)[C@]2(O)CCCC[C@@H]2O1)c1c[nH]cc(-c2ccccc2)c1=O] | 461.5 | 462.1 | 0.55 |
| 172 | 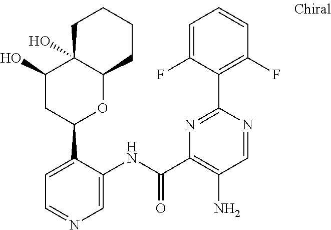 [SMILES: Nc1cnc(-c2c(F)cccc2F)nc1C(=O)Nc1cnccc1[C@H]1C[C@@H](O)[C@@]2(O)CCCC[C@H]2O1] | 497.5 | 498.1 | 0.59 |
| 173 | 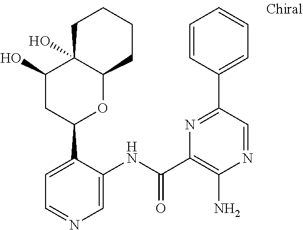 [SMILES: Nc1ncc(-c2ccccc2)nc1C(=O)Nc1cnccc1[C@H]1C[C@@H](O)[C@@]2(O)CCCC[C@H]2O1] | 461.5 | 462.1 | 0.61 |
| 174 | 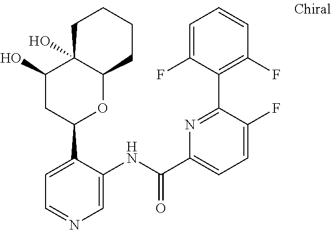 [SMILES: O=C(Nc1cnccc1[C@H]1C[C@@H](O)[C@@]2(O)CCCC[C@H]2O1)c1ccc(F)c(-c2c(F)cccc2F)n1] | 499.5 | 500.1 | 0.69 |
| 175 | 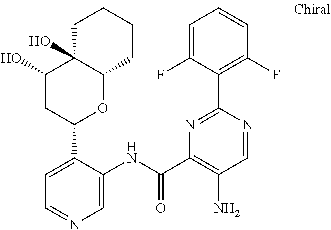 [SMILES: Nc1cnc(-c2c(F)cccc2F)nc1C(=O)Nc1cnccc1[C@@H]1C[C@H](O)[C@]2(O)CCCC[C@@H]2O1] | 497.5 | 498.1 | 0.58 |
| 176 | 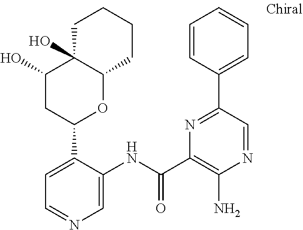 [SMILES: Nc1ncc(-c2ccccc2)nc1C(=O)Nc1cnccc1[C@@H]1C[C@H](O)[C@]2(O)CCCC[C@@H]2O1] | 461.5 | 462.1 | 0.61 |
| 177 | 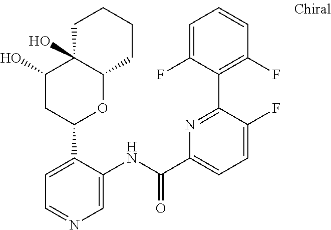 [SMILES: O=C(Nc1cnccc1[C@@H]1C[C@H](O)[C@]2(O)CCCC[C@@H]2O1)c1ccc(F)c(-c2c(F)cccc2F)n1] | 499.5 | 500.1 | 0.69 |
| 178 | 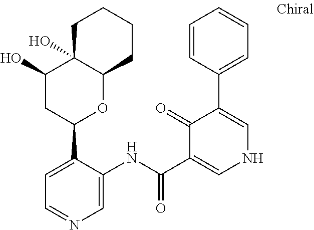 [SMILES: O=C(Nc1cnccc1[C@H]1C[C@@H](O)[C@@]2(O)CCCC[C@H]2O1)c1c[nH]cc(-c2ccccc2)c1=O] | 461.5 | 462.1 | 0.55 |
| 179 | 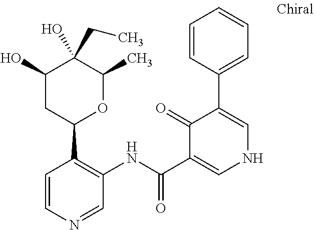 [SMILES: CC[C@]1(O)[C@H](O)C[C@H](c2ccncc2NC(=O)c2c[nH]cc(-c3ccccc3)c2=O)O[C@@H]1C] | 449.5 | 450.1 | 0.54 |
| 180 | 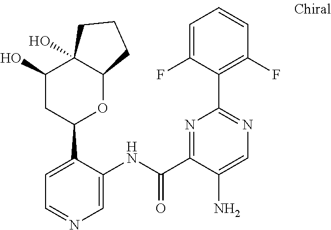 [SMILES: Nc1cnc(-c2c(F)cccc2F)nc1C(=O)Nc1cnccc1[C@H]1C[C@@H](O)[C@@]2(O)CCC[C@H]2O1] | 483.5 | 484.1 | 0.53 |
| 181 | 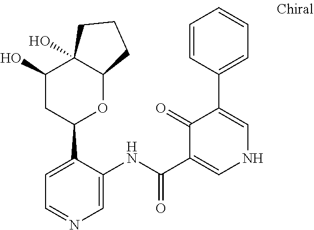 [SMILES: O=C(Nc1cnccc1[C@H]1C[C@@H](O)[C@@]2(O)CCC[C@H]2O1)c1c[nH]cc(-c2ccccc2)c1=O] | 447.5 | 448.1 | 0.48 |
| 182 | 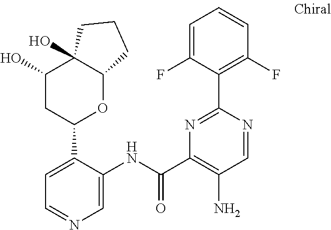 [SMILES: Nc1cnc(-c2c(F)cccc2F)nc1C(=O)Nc1cnccc1[C@@H]1C[C@H](O)[C@]2(O)CCC[C@@H]2O1] | 483.5 | 484.1 | 0.53 |
| 183 | 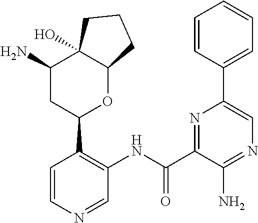 [SMILES: Nc1ncc(-c2ccccc2)nc1C(=O)Nc1cnccc1[C@H]1C[C@@H](N)[C@@]2(O)CCC[C@H]2O1] | 446.5 | 447.1 | 0.54 |
| 184 | 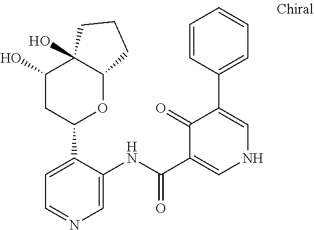 [SMILES: O=C(Nc1cnccc1[C@@H]1C[C@H](O)[C@]2(O)CCC[C@@H]2O1)c1c[nH]cc(-c2ccccc2)c1=O] | 447.5 | 448.1 | 0.49 |
| 185 | 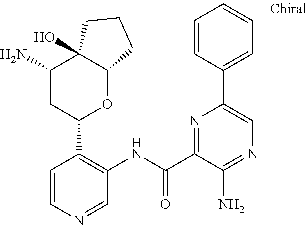 [SMILES: Nc1ncc(-c2ccccc2)nc1C(=O)Nc1cnccc1[C@@H]1C[C@H](N)[C@]2(O)CCC[C@@H]2O1] | 446.5 | 447.1 | 0.54 |
| 186 | 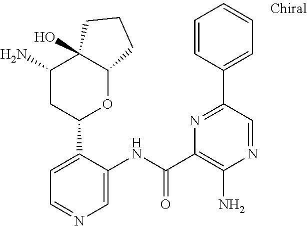 [SMILES: Nc1ncc(-c2ccccc2)nc1C(=O)Nc1cnccc1[C@@H]1C[C@H](N)[C@]2(O)CCC[C@@H]2O1] | 447.5 | 448.1 | 0.57 |
| 187 | 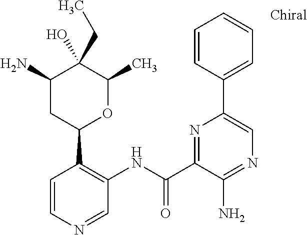 [SMILES: CC[C@]1(O)[C@H](N)C[C@H](c2ccncc2NC(=O)c2nc(-c3ccccc3)cnc2N)O[C@@H]1C] | 448.5 | 449.1 | 0.61 |
| 188 | 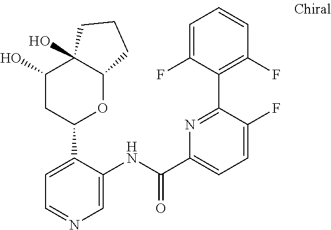 [SMILES: O=C(Nc1cnccc1[C@@H]1C[C@H](O)[C@]2(O)CCC[C@@H]2O1)c1ccc(F)c(-c2c(F)cccc2F)n1] | 485.5 | 486.1 | 0.65 |
| 189 | 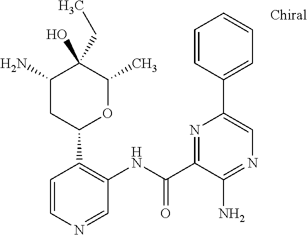 [SMILES: CC[C@]1(O)[C@H](C)O[C@H](c2ccncc2NC(=O)c2nc(-c3ccccc3)cnc2N)C[C@@H]1N] | 448.5 | 449.2 | 0.58 |
| 190 | 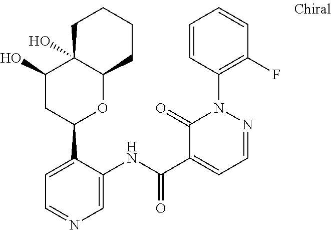 [SMILES: O=C(Nc1cnccc1[C@H]1C[C@@H](O)[C@@]2(O)CCCC[C@H]2O1)c1ccnn(-c2ccccc2F)c1=O] | 480.5 | 481.1 | 0.59 |
| 191 | 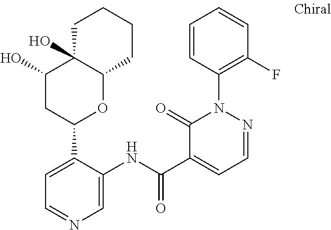 [SMILES: O=C(Nc1cnccc1[C@@H]1C[C@H](O)[C@]2(O)CCCC[C@@H]2O1)c1ccnn(-c2ccccc2F)c1=O] | 480.5 | 481.1 | 0.59 |
| 192 | 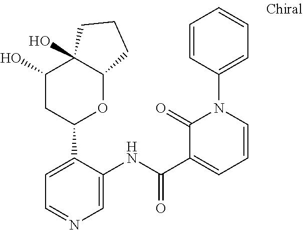 [SMILES: O=C(Nc1cnccc1[C@@H]1C[C@H](O)[C@]2(O)CCC[C@@H]2O1)c1cccn(-c2ccccc2)c1=O] | 447.2 | 448.1 | 0.50 |
| 193 | 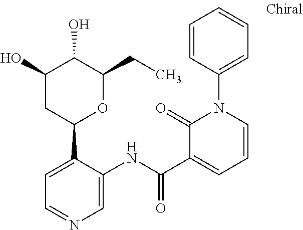 [SMILES: CC[C@H]1O[C@@H](c2ccncc2NC(=O)c2cccn(-c3ccccc3)c2=O)C[C@@H](O)[C@@H]1O] | 435.5 | 436.1 | 0.52 |
| 194 | 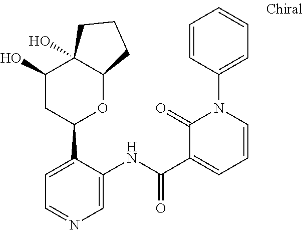 [SMILES: O=C(Nc1cnccc1[C@H]1C[C@@H](O)[C@@]2(O)CCC[C@H]2O1)c1cccn(-c2ccccc2)c1=O] | 447.5 | 448.1 | 0.50 |
| 195 | 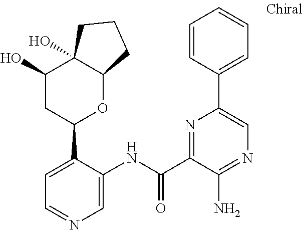 [SMILES: Nc1ncc(-c2ccccc2)nc1C(=O)Nc1cnccc1[C@H]1C[C@@H](O)[C@@]2(O)CCC[C@H]2O1] | 447.5 | 448.1 | 0.55 |
| 196 | 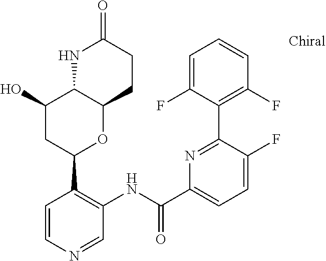 [SMILES: O=C1CC[C@H]2O[C@@H](c3ccncc3NC(=O)c3ccc(F)c(-c4c(F)cccc4F)n3)C[C@@H](O)[C@@H]2N1] | 498.5 | 499.1 | 0.55 |
| 197 | 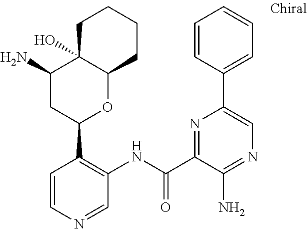 [SMILES: Nc1ncc(-c2ccccc2)nc1C(=O)Nc1cnccc1[C@H]1C[C@@H](N)[C@@]2(O)CCCC[C@H]2O1] | 460.5 | 461.1 | 0.58 |
| 198 | 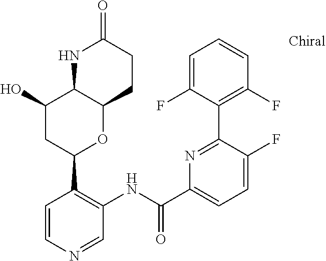 [SMILES: O=C1CC[C@H]2O[C@@H](c3ccncc3NC(=O)c3ccc(F)c(-c4c(F)cccc4F)n3)C[C@@H](O)[C@H]2N1] | 498.5 | 499.1 | 0.59 |
| 199 | 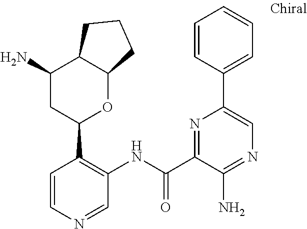 [SMILES: Nc1ncc(-c2ccccc2)nc1C(=O)Nc1cnccc1[C@H]1C[C@@H](N)[C@H]2CCC[C@H]2O1] | 430.5 | 431.0 | 0.58 |
| 200 | 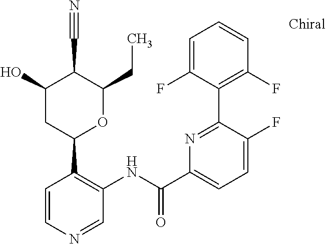 [SMILES: CC[C@H]1O[C@@H](c2ccncc2NC(=O)c2ccc(F)c(-c3c(F)cccc3F)n2)C[C@@H](O)[C@H]1C#N] | 482.5 | 483.2 | 0.71 |
| 201 | 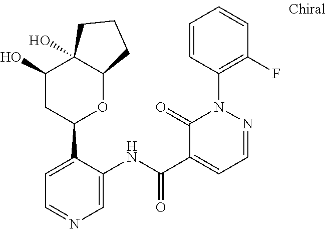 [SMILES: O=C(Nc1cnccc1[C@H]1C[C@@H](O)[C@@]2(O)CCC[C@H]2O1)c1ccnn(-c2ccccc2F)c1=O] | 466.5 | 467.1 | 0.53 |
| 202 | 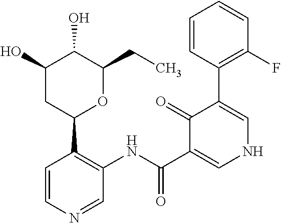 [SMILES: CC[C@H]1O[C@@H](c2ccncc2NC(=O)c2c[nH]cc(-c3ccccc3F)c2=O)C[C@@H](O)[C@@H]1O] | 453.5 | 454.1 | 0.50 |
| 203 | 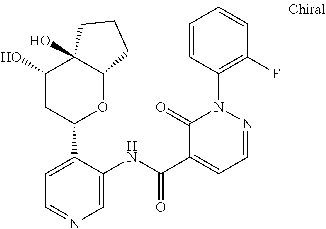 [SMILES: O=C(Nc1cnccc1[C@@H]1C[C@H](O)[C@]2(O)CCC[C@@H]2O1)c1ccnn(-c2ccccc2F)c1=O] | 466.5 | 467.1 | 0.52 |
| 204 | 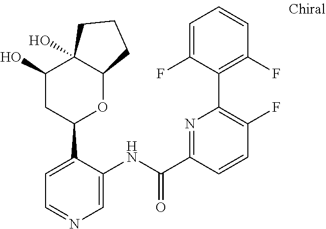 [SMILES: O=C(Nc1cnccc1[C@H]1C[C@@H](O)[C@@]2(O)CCC[C@H]2O1)c1ccc(F)c(-c2c(F)cccc2F)n1] | 485.5 | 486.1 | 0.63 |
| 205 | 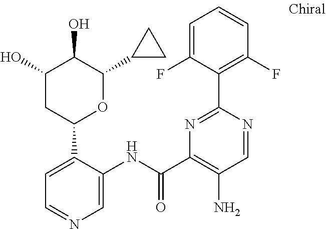 [SMILES: Nc1cnc(-c2c(F)cccc2F)nc1C(=O)Nc1cnccc1[C@@H]1C[C@H](O)[C@@H](O)[C@H](C2CC2)O1] | 483.2 | 484.1 | 0.53 |
| 206 | 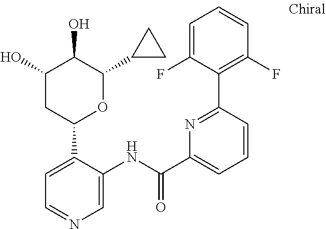 [SMILES: O=C(Nc1cnccc1[C@@H]1C[C@H](O)[C@@H](O)[C@H](C2CC2)O1)c1cccc(-c2c(F)cccc2F)n1] | 467.2 | 468.1 | 0.62 |
| 207 | 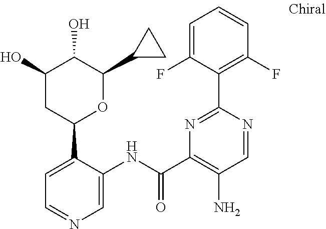 [SMILES: Nc1cnc(-c2c(F)cccc2F)nc1C(=O)Nc1cnccc1[C@H]1C[C@@H](O)[C@H](O)[C@@H](C2CC2)O1] | 483.2 | 484.1 | 0.53 |
| 208 | 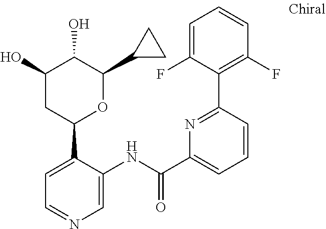 [SMILES: O=C(Nc1cnccc1[C@H]1C[C@@H](O)[C@H](O)[C@@H](C2CC2)O1)c1cccc(-c2c(F)cccc2F)n1] | 467.2 | 468.1 | 0.62 |
| 209 | 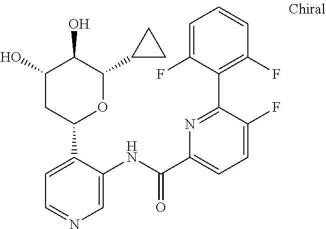 [SMILES: O=C(Nc1cnccc1[C@@H]1C[C@H](O)[C@@H](O)[C@H](C2CC2)O1)c1ccc(F)c(-c2c(F)cccc2F)n1] | 485.2 | 486.1 | 0.63 |
| 210 | 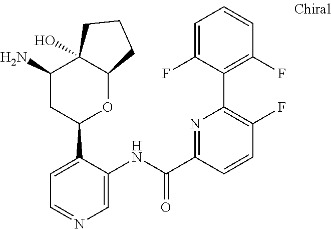 [SMILES: N[C@@H]1C[C@H](c2ccncc2NC(=O)c2ccc(F)c(-c3c(F)cccc3F)n2)O[C@@H]2CCC[C@]12O] | 484.5 | 485.1 | 0.57 |
| 211 | 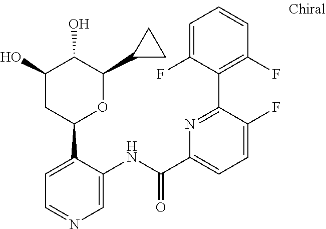 [SMILES: O=C(Nc1cnccc1[C@H]1C[C@@H](O)[C@H](O)[C@@H](C2CC2)O1)c1ccc(F)c(-c2c(F)cccc2F)n1] | 485.2 | 486.1 | 0.62 |
| 212 | 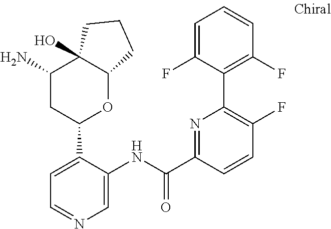 [SMILES: N[C@H]1C[C@@H](c2ccncc2NC(=O)c2ccc(F)c(-c3c(F)cccc3F)n2)O[C@H]2CCC[C@]21O] | 484.5 | 485.1 | 0.57 |
| 213 | 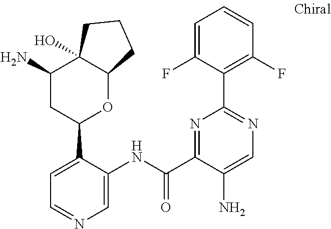 [SMILES: Nc1cnc(-c2c(F)cccc2F)nc1C(=O)Nc1cnccc1[C@H]1C[C@@H](N)[C@@]2(O)CCC[C@H]2O1] | 482.5 | 483.1 | 0.47 |
| 214 | 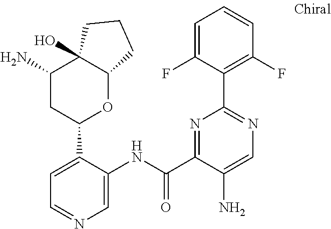 [SMILES: Nc1cnc(-c2c(F)cccc2F)nc1C(=O)Nc1cnccc1[C@@H]1C[C@H](N)[C@]2(O)CCC[C@@H]2O1] | 482.5 | 483.1 | 0.47 |
| 215 | 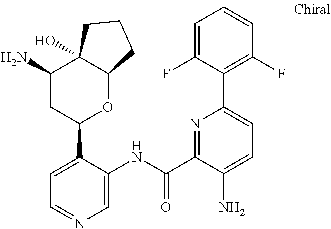 [SMILES: Nc1ccc(-c2c(F)cccc2F)nc1C(=O)Nc1cnccc1[C@H]1C[C@@H](N)[C@@]2(O)CCC[C@H]2O1] | 481.5 | 482.1 | 0.54 |
| 216 | 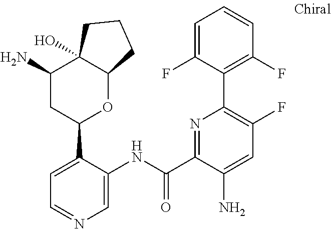 [SMILES: Nc1cc(F)c(-c2c(F)cccc2F)nc1C(=O)Nc1cnccc1[C@H]1C[C@@H](N)[C@@]2(O)CCC[C@H]2O1] | 499.5 | 500.1 | 0.56 |
| 218 | 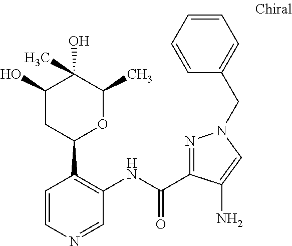 [SMILES: C[C@H]1O[C@@H](c2ccncc2NC(=O)c2nn(Cc3ccccc3)cc2N)C[C@@H](O)[C@]1(C)O] | 437.5 | 438.2 | 0.46 |
| 219 | 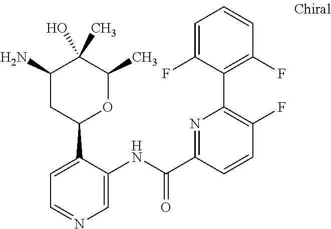 [SMILES: C[C@H]1O[C@@H](c2ccncc2NC(=O)c2ccc(F)c(-c3c(F)cccc3F)n2)C[C@@H](N)[C@]1(C)O] | 472.5 | 473.1 | 0.56 |
| 220 | 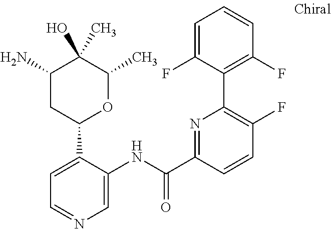 [SMILES: C[C@@H]1O[C@H](c2ccncc2NC(=O)c2ccc(F)c(-c3c(F)cccc3F)n2)C[C@H](N)[C@@]1(C)O] | 472.5 | 473.1 | 0.56 |
| 221 | 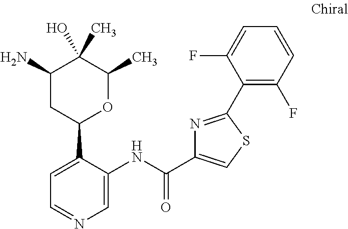 [SMILES: C[C@H]1O[C@@H](c2ccncc2NC(=O)c2csc(-c3c(F)cccc3F)n2)C[C@@H](N)[C@]1(C)O] | 460.5 | 461.1 | 0.53 |
| 222 | 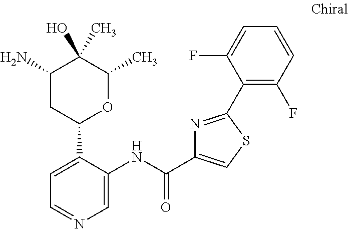 [SMILES: C[C@@H]1O[C@H](c2ccncc2NC(=O)c2csc(-c3c(F)cccc3F)n2)C[C@H](N)[C@@]1(C)O] | 460.5 | 461.1 | 0.53 |
| 223 | 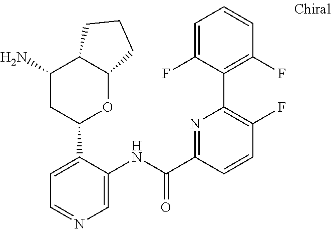 [SMILES: N[C@H]1C[C@@H](c2ccncc2NC(=O)c2ccc(F)c(-c3c(F)cccc3F)n2)O[C@H]2CCC[C@H]21] | 468.5 | 469.1 | 0.60 |
| 224 | 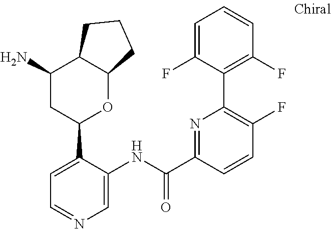 [SMILES: N[C@@H]1C[C@H](c2ccncc2NC(=O)c2ccc(F)c(-c3c(F)cccc3F)n2)O[C@@H]2CCC[C@H]12] | 468.5 | 469.1 | 0.60 |
| 225 | 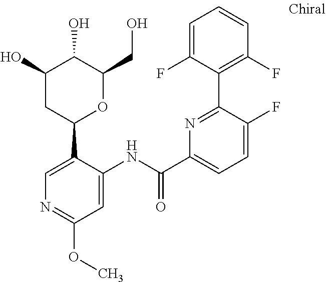 [SMILES: COc1cc(NC(=O)c2ccc(F)c(-c3c(F)cccc3F)n2)c([C@H]2C[C@@H](O)[C@H](O)[C@@H](CO)O2)cn1] | 505.4 | 506.1 | 0.64 |
| 226 | 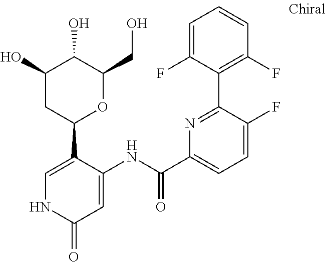 [SMILES: O=C(Nc1cc(=O)[nH]cc1[C@H]1C[C@@H](O)[C@H](O)[C@@H](CO)O1)c1ccc(F)c(-c2c(F)cccc2F)n1] | 491.4 | 492.1 | 0.57 |
| 227 | 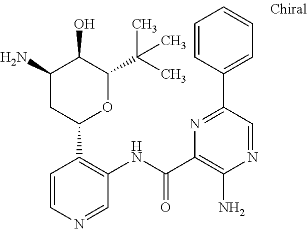 [SMILES: CC(C)(C)[C@@H]1O[C@H](c2ccncc2NC(=O)c2nc(-c3ccccc3)cnc2N)C[C@@H](N)[C@H]1O] | 462.6 | 463.1 | 0.65 |
| 228 | 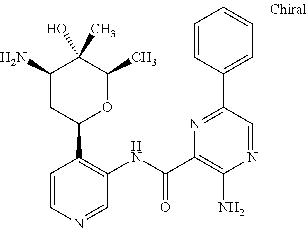 [SMILES: C[C@H]1O[C@@H](c2ccncc2NC(=O)c2nc(-c3ccccc3)cnc2N)C[C@@H](N)[C@]1(C)O] | 434.5 | 435.0 | 0.56 |
| 229 | 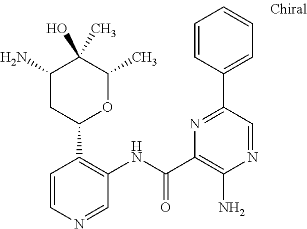 [SMILES: C[C@@H]1O[C@H](c2ccncc2NC(=O)c2nc(-c3ccccc3)cnc2N)C[C@H](N)[C@@]1(C)O] | 434.5 | 435.0 | 0.56 |
| 230 | 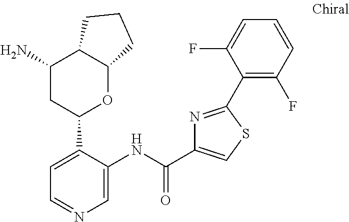 [SMILES: N[C@H]1C[C@@H](c2ccncc2NC(=O)c2csc(-c3c(F)cccc3F)n2)O[C@H]2CCC[C@H]21] | 456.5 | 457.1 | 0.57 |
| 231 | 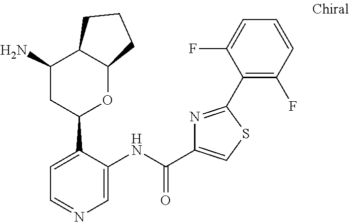 [SMILES: N[C@@H]1C[C@H](c2ccncc2NC(=O)c2csc(-c3c(F)cccc3F)n2)O[C@@H]2CCC[C@H]12] | 456.5 | 457.1 | 0.57 |
| 232 | 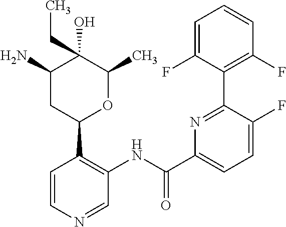 [SMILES: CC[C@]1(O)[C@H](N)C[C@H](c2ccncc2NC(=O)c2ccc(F)c(-c3c(F)cccc3F)n2)O[C@@H]1C] | 486.5 | 487.1 | 0.64 |
| 233 | 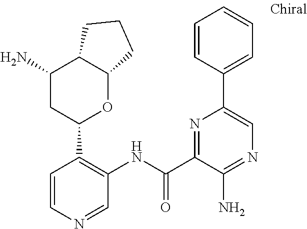 [SMILES: Nc1ncc(-c2ccccc2)nc1C(=O)Nc1cnccc1[C@@H]1C[C@H](N)[C@@H]2CCC[C@@H]2O1] | 430.5 | 431.1 | 0.60 |
| 234 | 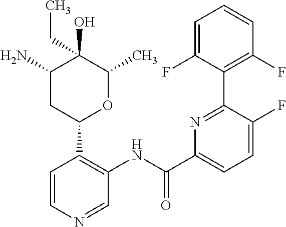 [SMILES: CC[C@]1(O)[C@H](C)O[C@H](c2ccncc2NC(=O)c2ccc(F)c(-c3c(F)cccc3F)n2)C[C@@H]1N] | 486.5 | 487.1 | 0.60 |
| 235 | 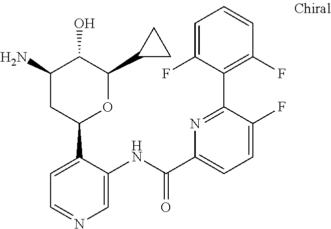 [SMILES: N[C@@H]1C[C@H](c2ccncc2NC(=O)c2ccc(F)c(-c3c(F)cccc3F)n2)O[C@H](C2CC2)[C@H]1O] | 484.2 | 485.1 | 0.59 |
| 236 | 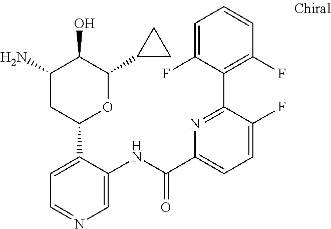 [SMILES: N[C@H]1C[C@@H](c2ccncc2NC(=O)c2ccc(F)c(-c3c(F)cccc3F)n2)O[C@@H](C2CC2)[C@@H]1O] | 484.2 | 485.1 | 0.59 |
| 237 | 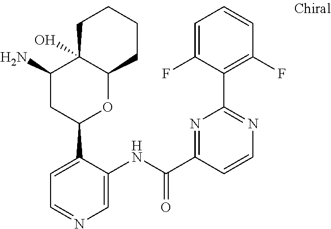 [SMILES: N[C@@H]1C[C@H](c2ccncc2NC(=O)c2ccnc(-c3c(F)cccc3F)n2)O[C@@H]2CCCC[C@]12O] | 481.5 | 482.1 | 0.57 |
| 238 | 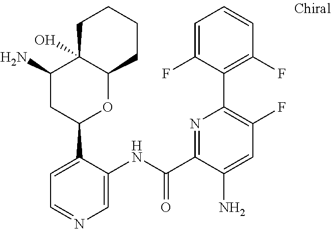 [SMILES: Nc1cc(F)c(-c2c(F)cccc2F)nc1C(=O)Nc1cnccc1[C@H]1C[C@@H](N)[C@@]2(O)CCCC[C@H]2O1] | 513.5 | 514.1 | 0.66 |
| 239 | 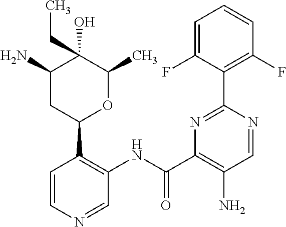 [SMILES: CC[C@]1(O)[C@H](N)C[C@H](c2ccncc2NC(=O)c2nc(-c3c(F)cccc3F)ncc2N)O[C@@H]1C] | 484.5 | 485.1 | 0.53 |
| 240 | 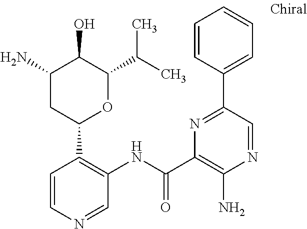 [SMILES: CC(C)[C@@H]1O[C@H](c2ccncc2NC(=O)c2nc(-c3ccccc3)cnc2N)C[C@H](N)[C@H]1O] | 448.5 | 449.1 | 0.62 |
| 241 | 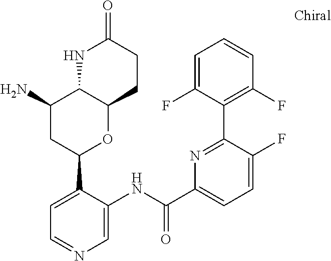 [SMILES: N[C@@H]1C[C@H](c2ccncc2NC(=O)c2ccc(F)c(-c3c(F)cccc3F)n2)O[C@@H]2CCC(=O)N[C@@H]12] | 497.5 | 498.1 | 0.55 |
| 242 | 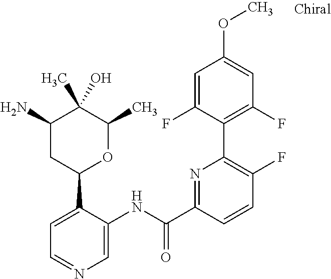 [SMILES: COc1cc(F)c(-c2nc(C(=O)Nc3cnccc3[C@H]3C[C@@H](N)[C@](C)(O)[C@@H](C)O3)ccc2F)c(F)c1] | 502.5 | 503.1 | 0.62 |
| 243 | 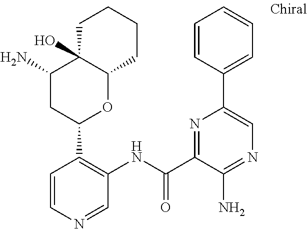 [SMILES: Nc1ncc(-c2ccccc2)nc1C(=O)Nc1cnccc1[C@@H]1C[C@H](N)[C@]2(O)CCCC[C@@H]2O1] | 460.5 | 461.2 | 0.60 |
| 244 | 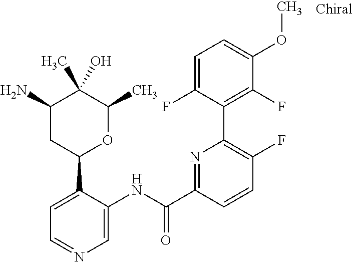 [SMILES: COc1ccc(F)c(-c2nc(C(=O)Nc3cnccc3[C@H]3C[C@@H](N)[C@](C)(O)[C@@H](C)O3)ccc2F)c1F] | 502.5 | 503.1 | 0.59 |
| 245 | 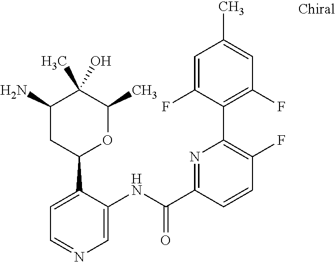 [SMILES: Cc1cc(F)c(-c2nc(C(=O)Nc3cnccc3[C@H]3C[C@@H](N)[C@](C)(O)[C@@H](C)O3)ccc2F)c(F)c1] | 486.5 | 487.1 | 0.63 |
| 246 | 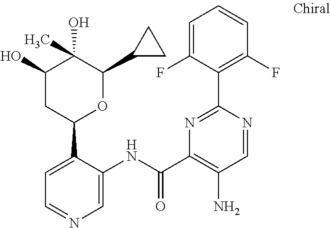 [SMILES: C[C@]1(O)[C@H](O)C[C@H](c2ccncc2NC(=O)c2nc(-c3c(F)cccc3F)ncc2N)O[C@@H]1C1CC1] | 497.2 | 498.1 | 0.55 |
| 247 | 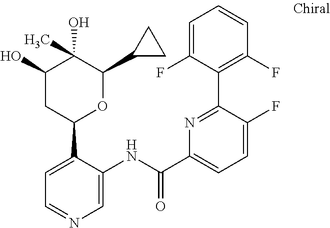 [SMILES: C[C@]1(O)[C@H](O)C[C@H](c2ccncc2NC(=O)c2ccc(F)c(-c3c(F)cccc3F)n2)O[C@@H]1C1CC1] | 499.2 | 500.0 | 0.65 |
| 248 | 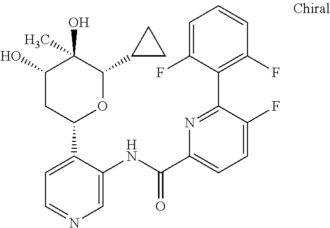 [SMILES: C[C@@]1(O)[C@@H](O)C[C@@H](c2ccncc2NC(=O)c2ccc(F)c(-c3c(F)cccc3F)n2)O[C@H]1C1CC1] | 499.2 | 500.0 | 0.65 |
| 249 | 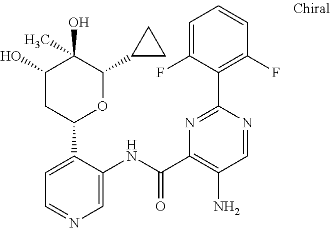 [SMILES: C[C@@]1(O)[C@@H](O)C[C@@H](c2ccncc2NC(=O)c2nc(-c3c(F)cccc3F)ncc2N)O[C@H]1C1CC1] | 497.2 | 498.1 | 0.55 |
| 250 | 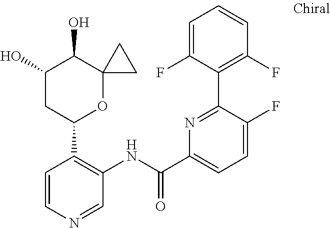 [SMILES: O=C(Nc1cnccc1[C@@H]1C[C@H](O)[C@@H](O)C2(CC2)O1)c1ccc(F)c(-c2c(F)cccc2F)n1] | 471.4 | 472.1 | 0.67 |
| 251 | 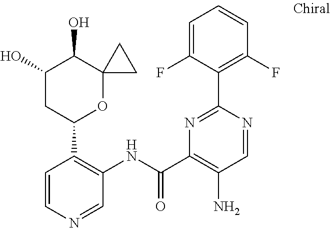 [SMILES: Nc1cnc(-c2c(F)cccc2F)nc1C(=O)Nc1cnccc1[C@@H]1C[C@H](O)[C@@H](O)C2(CC2)O1] | 469.4 | 470.0 | 0.56 |
| 252 | 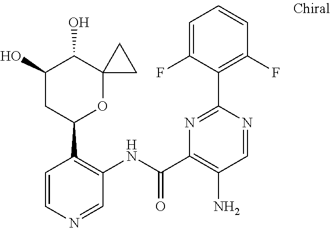 [SMILES: Nc1cnc(-c2c(F)cccc2F)nc1C(=O)Nc1cnccc1[C@H]1C[C@@H](O)[C@H](O)C2(CC2)O1] | 469.4 | 470.0 | 0.56 |
| 253 | 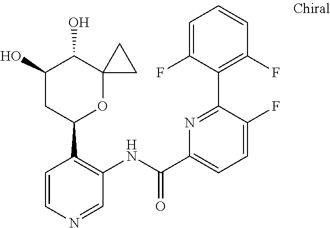 [SMILES: O=C(Nc1cnccc1[C@H]1C[C@@H](O)[C@H](O)C2(CC2)O1)c1ccc(F)c(-c2c(F)cccc2F)n1] | 471.4 | 472.1 | 0.67 |
| 254 | 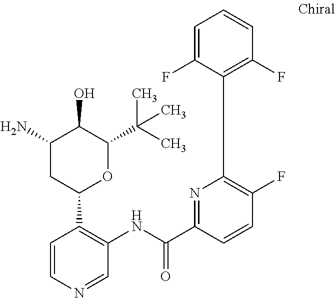 [SMILES: CC(C)(C)[C@@H]1O[C@H](c2ccncc2NC(=O)c2ccc(F)c(-c3c(F)cccc3F)n2)C[C@H](N)[C@H]1O] | 500.5 | 501.1 | 0.67 |
| 255 | 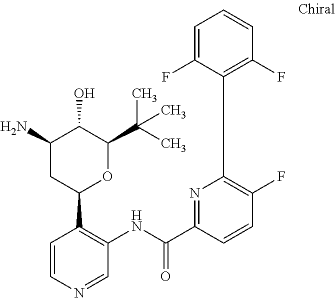 [SMILES: CC(C)(C)[C@H]1O[C@@H](c2ccncc2NC(=O)c2ccc(F)c(-c3c(F)cccc3F)n2)C[C@@H](N)[C@@H]1O] | 500.5 | 501.1 | 0.68 |
| 256 | 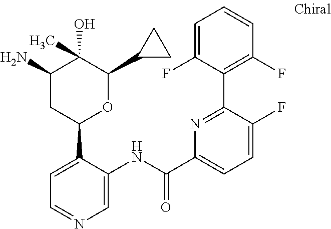 [SMILES: C[C@]1(O)[C@H](N)C[C@H](c2ccncc2NC(=O)c2ccc(F)c(-c3c(F)cccc3F)n2)O[C@@H]1C1CC1] | 498.2 | 499.1 | 0.60 |
| 257 | 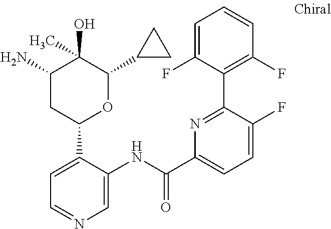 [SMILES: C[C@@]1(O)[C@@H](N)C[C@@H](c2ccncc2NC(=O)c2ccc(F)c(-c3c(F)cccc3F)n2)O[C@H]1C1CC1] | 498.2 | 499.1 | 0.60 |
| 258 | 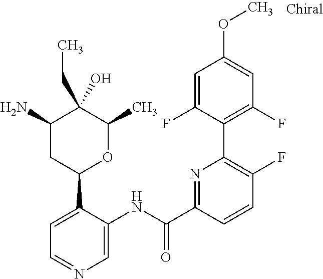 [SMILES: CC[C@]1(O)[C@H](N)C[C@H](c2ccncc2NC(=O)c2ccc(F)c(-c3c(F)cc(OC)cc3F)n2)O[C@@H]1C] | 516.5 | 517.0 | 0.65 |
| 259 | 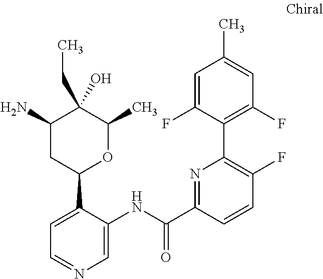 [SMILES: CC[C@]1(O)[C@H](N)C[C@H](c2ccncc2NC(=O)c2ccc(F)c(-c3c(F)cc(C)cc3F)n2)O[C@@H]1C] | 500.5 | 501.0 | 0.67 |
| 260 | 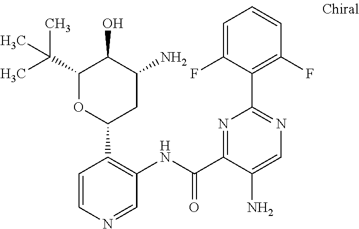 [SMILES: CC(C)(C)[C@H]1O[C@@H](c2ccncc2NC(=O)c2nc(-c3c(F)cccc3F)ncc2N)C[C@@H](N)[C@@H]1O] | 498.5 | 499.1 | 0.61 |
| 261 | 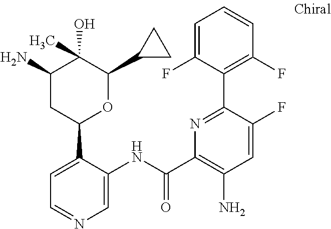 [SMILES: C[C@]1(O)[C@H](N)C[C@H](c2ccncc2NC(=O)c2nc(-c3c(F)cccc3F)c(F)cc2N)O[C@@H]1C1CC1] | 513.5 | 514.1 | 0.64 |
| 262 | 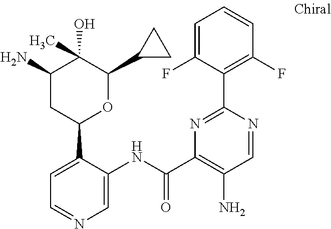 [SMILES: C[C@]1(O)[C@H](N)C[C@H](c2ccncc2NC(=O)c2nc(-c3c(F)cccc3F)ncc2N)O[C@@H]1C1CC1] | 496.5 | 497.1 | 0.54 |
| 263 | 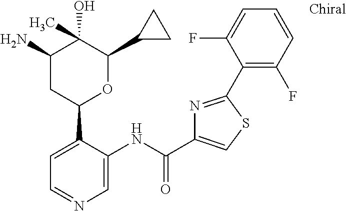 [SMILES: C[C@]1(O)[C@H](N)C[C@H](c2ccncc2NC(=O)c2csc(-c3c(F)cccc3F)n2)O[C@@H]1C1CC1] | 486.5 | 487.1 | 0.60 |
| 264 | 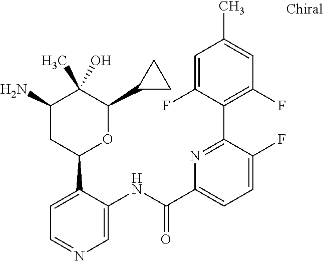 [SMILES: Cc1cc(F)c(-c2nc(C(=O)Nc3cnccc3[C@H]3C[C@@H](N)[C@](C)(O)[C@@H](C4CC4)O3)ccc2F)c(F)c1] | 512.5 | 513.1 | 0.71 |
| 265 | 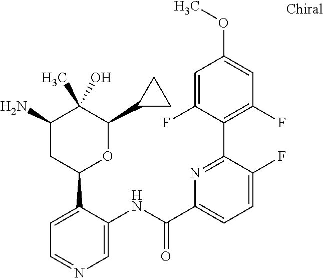 [SMILES: COc1cc(F)c(-c2nc(C(=O)Nc3cnccc3[C@H]3C[C@@H](N)[C@](C)(O)[C@@H](C4CC4)O3)ccc2F)c(F)c1] | 528.5 | 529.1 | 0.71 |
| 266 | 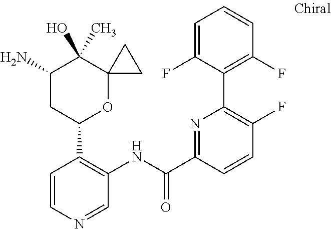 [SMILES: C[C@@]1(O)[C@@H](N)C[C@@H](c2ccncc2NC(=O)c2ccc(F)c(-c3c(F)cccc3F)n2)OC12CC2] | 484.5 | 485.0 | 0.61 |
| 267 | 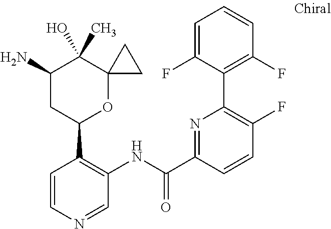 [SMILES: C[C@]1(O)[C@H](N)C[C@H](c2ccncc2NC(=O)c2ccc(F)c(-c3c(F)cccc3F)n2)OC12CC2] | 484.5 | 485.0 | 0.60 |
| 268 | 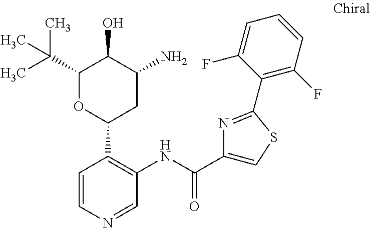 [SMILES: CC(C)(C)[C@H]1O[C@@H](c2ccncc2NC(=O)c2csc(-c3c(F)cccc3F)n2)C[C@@H](N)[C@@H]1O] | 488.6 | 489.1 | 0.64 |
| 269 | 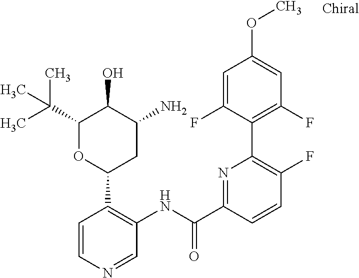 [SMILES: COc1cc(F)c(-c2nc(C(=O)Nc3cnccc3[C@H]3C[C@@H](N)[C@H](O)[C@@H](C(C)(C)C)O3)ccc2F)c(F)c1] | 530.5 | 531.1 | 0.71 |
| 270 | 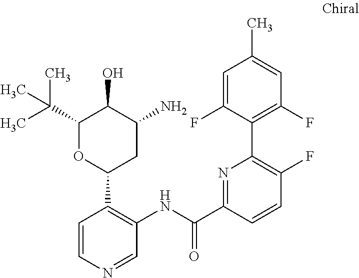 [SMILES: Cc1cc(F)c(-c2nc(C(=O)Nc3cnccc3[C@H]3C[C@@H](N)[C@H](O)[C@@H](C(C)(C)C)O3)ccc2F)c(F)c1] | 514.5 | 515.1 | 0.72 |
| 271 | 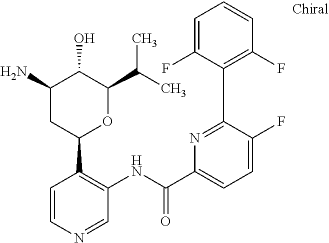 [SMILES: CC(C)[C@H]1O[C@@H](c2ccncc2NC(=O)c2ccc(F)c(-c3c(F)cccc3F)n2)C[C@@H](N)[C@@H]1O] | 486.5 | 487.1 | 0.63 |
| 272 | 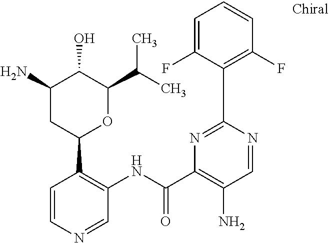 [SMILES: CC(C)[C@H]1O[C@@H](c2ccncc2NC(=O)c2nc(-c3c(F)cccc3F)ncc2N)C[C@@H](N)[C@@H]1O] | 484.5 | 485.1 | 0.54 |
| 273 | 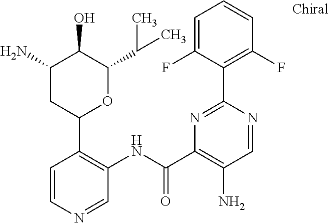 [SMILES: CC(C)[C@@H]1OC(c2ccncc2NC(=O)c2nc(-c3c(F)cccc3F)ncc2N)C[C@H](N)[C@H]1O] | 484.5 | 485.1 | 0.56 |
| 274 | 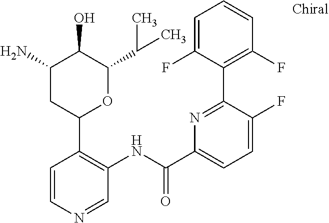 [SMILES: CC(C)[C@@H]1OC(c2ccncc2NC(=O)c2ccc(F)c(-c3c(F)cccc3F)n2)C[C@H](N)[C@H]1O] | 486.5 | 487.1 | 0.64 |
| 275 | 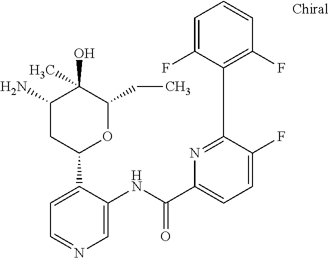 [SMILES: CC[C@@H]1O[C@H](c2ccncc2NC(=O)c2ccc(F)c(-c3c(F)cccc3F)n2)C[C@H](N)[C@@]1(C)O] | 486.5 | 487.1 | 0.60 |
| 276 | 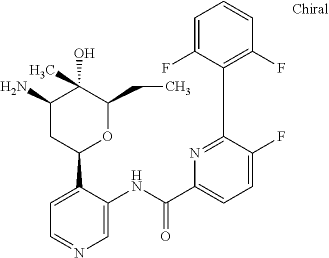 [SMILES: CC[C@H]1O[C@@H](c2ccncc2NC(=O)c2ccc(F)c(-c3c(F)cccc3F)n2)C[C@@H](N)[C@]1(C)O] | 486.5 | 487.1 | 0.61 |
| 277 | 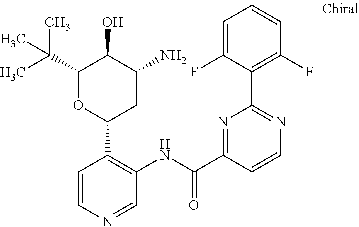 [SMILES: CC(C)(C)[C@H]1O[C@@H](c2ccncc2NC(=O)c2ccnc(-c3c(F)cccc3F)n2)C[C@@H](N)[C@@H]1O] | 483.5 | 484.1 | 0.59 |
| 278 | 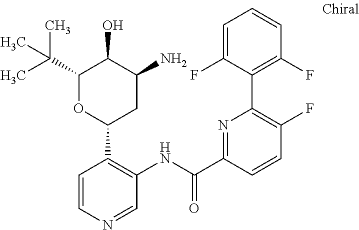 [SMILES: CC(C)(C)[C@H]1O[C@@H](c2ccncc2NC(=O)c2ccc(F)c(-c3c(F)cccc3F)n2)C[C@H](N)[C@@H]1O] | 500.5 | 501.1 | 0.68 |
| 279 | 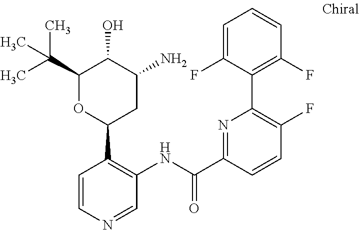 [SMILES: CC(C)(C)[C@@H]1O[C@H](c2ccncc2NC(=O)c2ccc(F)c(-c3c(F)cccc3F)n2)C[C@@H](N)[C@H]1O] | 500.5 | 501.0 | 0.68 |
| 280 | 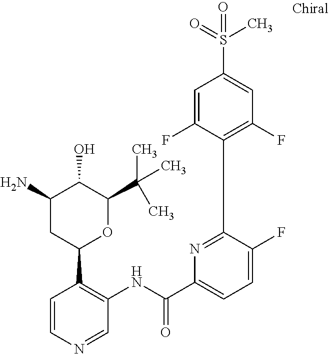 [SMILES: CC(C)(C)[C@H]1O[C@@H](c2ccncc2NC(=O)c2ccc(F)c(-c3c(F)cc(S(C)(=O)=O)cc3F)n2)C[C@@H](N)[C@@H]1O] | 578.2 | 579.0 | 0.62 |
| 281 | 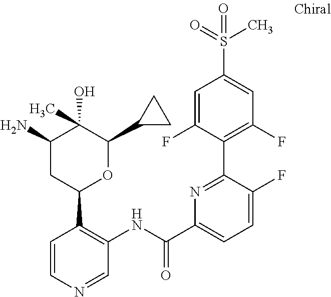 [SMILES: C[C@]1(O)[C@H](N)C[C@H](c2ccncc2NC(=O)c2ccc(F)c(-c3c(F)cc(S(C)(=O)=O)cc3F)n2)O[C@@H]1C1CC1] | 576.2 | 576.9 | 0.56 |
| 282 | 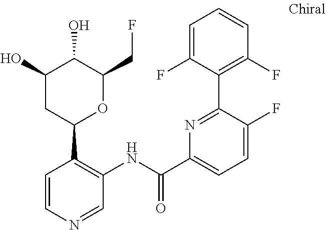 [SMILES: O=C(Nc1cnccc1[C@H]1C[C@@H](O)[C@H](O)[C@@H](CF)O1)c1ccc(F)c(-c2c(F)cccc2F)n1] | 477.4 | 478.1 | 0.62 |
| 283 | 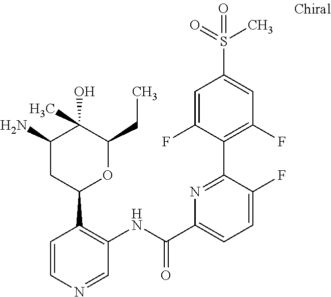 [SMILES: CC[C@H]1O[C@@H](c2ccncc2NC(=O)c2ccc(F)c(-c3c(F)cc(S(C)(=O)=O)cc3F)n2)C[C@@H](N)[C@]1(C)O] | 564.2 | 565.0 | 0.57 |
| 284 | 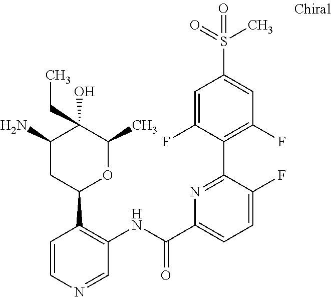 [SMILES: CC[C@]1(O)[C@H](N)C[C@H](c2ccncc2NC(=O)c2ccc(F)c(-c3c(F)cc(S(C)(=O)=O)cc3F)n2)O[C@@H]1C] | 564.2 | 564.9 | 0.58 |
| 285 | 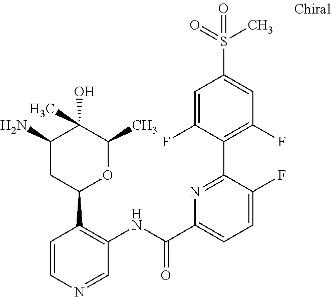 [SMILES: C[C@H]1O[C@@H](c2ccncc2NC(=O)c2ccc(F)c(-c3c(F)cc(S(C)(=O)=O)cc3F)n2)C[C@@H](N)[C@]1(C)O] | 550.1 | 551.0 | 0.56 |
| 286 | 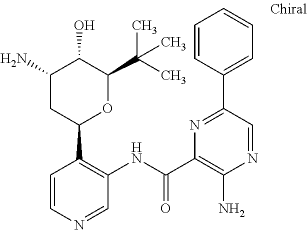 [SMILES: CC(C)(C)[C@H]1O[C@@H](c2ccncc2NC(=O)c2nc(-c3ccccc3)cnc2N)C[C@H](N)[C@@H]1O] | 462.6 | 463.1 | 0.65 |
| 287 | 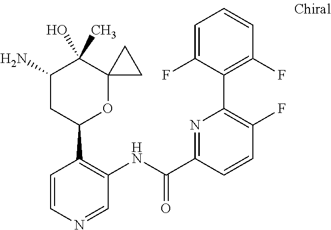 [SMILES: C[C@]1(O)[C@@H](N)C[C@H](c2ccncc2NC(=O)c2ccc(F)c(-c3c(F)cccc3F)n2)OC12CC2] | 484.5 | 485.0 | 0.62 |
| 288 | 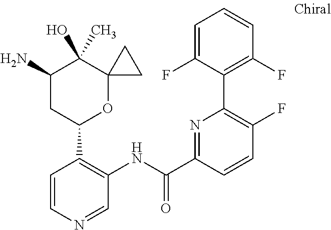 [SMILES: C[C@@]1(O)[C@H](N)C[C@@H](c2ccncc2NC(=O)c2ccc(F)c(-c3c(F)cccc3F)n2)OC12CC2] | 484.5 | 485.0 | 0.62 |
| 289 | 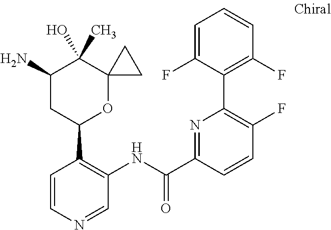 [SMILES: C[C@]1(O)[C@H](N)C[C@H](c2ccncc2NC(=O)c2ccc(F)c(-c3c(F)cccc3F)n2)OC12CC2] | 484.5 | 485.0 | 0.61 |
| 290 | 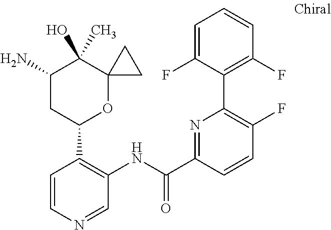 [SMILES: C[C@@]1(O)[C@@H](N)C[C@@H](c2ccncc2NC(=O)c2ccc(F)c(-c3c(F)cccc3F)n2)OC12CC2] | 484.5 | 485.0 | 0.61 |
| 291 | 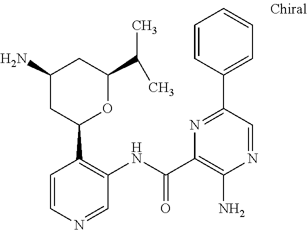 [SMILES: CC(C)[C@@H]1C[C@H](N)C[C@H](c2ccncc2NC(=O)c2nc(-c3ccccc3)cnc2N)O1] | 432.5 | 433.1 | 0.65 |
| 292 | 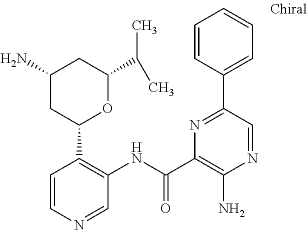 [SMILES: CC(C)[C@H]1C[C@@H](N)C[C@@H](c2ccncc2NC(=O)c2nc(-c3ccccc3)cnc2N)O1] | 432.5 | 433.1 | 0.65 |
| 293 | 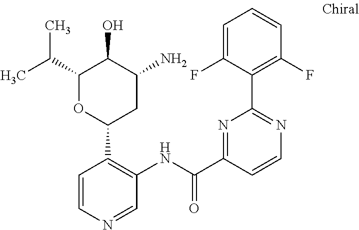 [SMILES: CC(C)[C@H]1O[C@@H](c2ccncc2NC(=O)c2ccnc(-c3c(F)cccc3F)n2)C[C@@H](N)[C@@H]1O] | 469.5 | 470.1 | 0.56 |
| 295 | 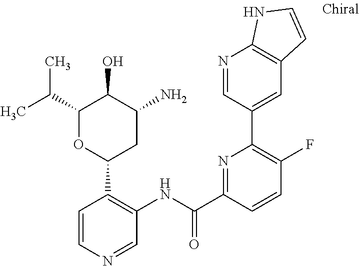 [SMILES: CC(C)[C@H]1O[C@@H](c2ccncc2NC(=O)c2ccc(F)c(-c3cnc4[nH]ccc4c3)n2)C[C@@H](N)[C@@H]1O] | 490.5 | 491.1 | 0.57 |
| 296 | 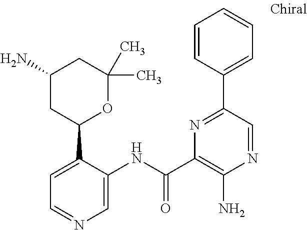 [SMILES: CC1(C)C[C@@H](N)C[C@H](c2ccncc2NC(=O)c2nc(-c3ccccc3)cnc2N)O1] | 418.5 | 419.1 | 0.55 |
| 297 | 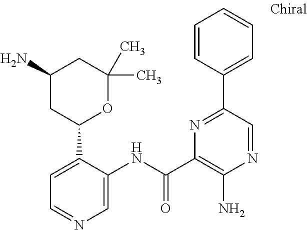 [SMILES: CC1(C)C[C@H](N)C[C@@H](c2ccncc2NC(=O)c2nc(-c3ccccc3)cnc2N)O1] | 418.5 | 419.1 | 0.55 |
| 298 | 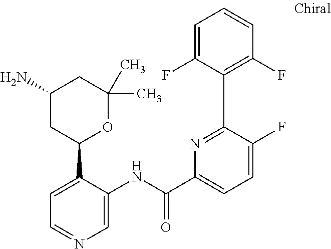 [SMILES: CC1(C)C[C@@H](N)C[C@H](c2ccncc2NC(=O)c2ccc(F)c(-c3c(F)cccc3F)n2)O1] | 456.5 | 457.1 | 0.61 |
| 299 | 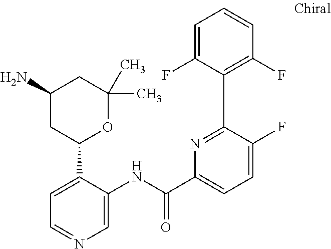 [SMILES: CC1(C)C[C@H](N)C[C@@H](c2ccncc2NC(=O)c2ccc(F)c(-c3c(F)cccc3F)n2)O1] | 456.5 | 457.1 | 0.61 |
| 300 | 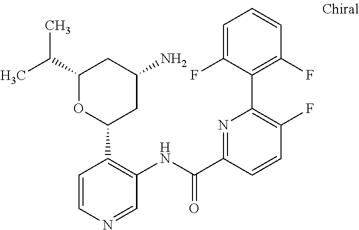 [SMILES: CC(C)[C@@H]1C[C@H](N)C[C@H](c2ccncc2NC(=O)c2ccc(F)c(-c3c(F)cccc3F)n2)O1] | 470.5 | 470.9 | 0.67 |
| 301 | 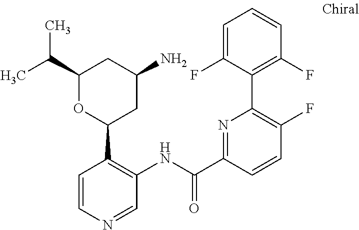 [SMILES: CC(C)[C@H]1C[C@@H](N)C[C@@H](c2ccncc2NC(=O)c2ccc(F)c(-c3c(F)cccc3F)n2)O1] | 470.5 | 470.9 | 0.67 |
| 302 | 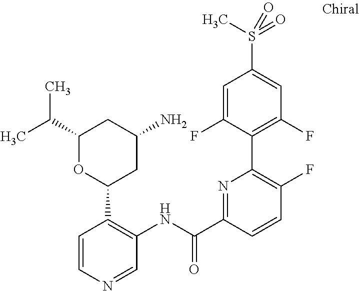 [SMILES: CC(C)[C@@H]1C[C@H](N)C[C@H](c2ccncc2NC(=O)c2ccc(F)c(-c3c(F)cc(S(C)(=O)=O)cc3F)n2)O1] | 548.6 | 548.9 | 0.62 |
| 303 | 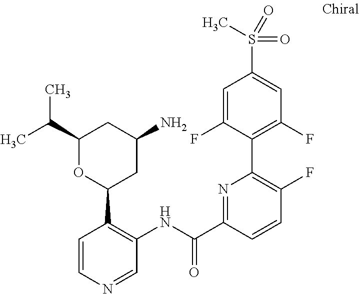 [SMILES: CC(C)[C@H]1C[C@@H](N)C[C@@H](c2ccncc2NC(=O)c2ccc(F)c(-c3c(F)cc(S(C)(=O)=O)cc3F)n2)O1] | 548.6 | 548.9 | 0.62 |
| 304 | 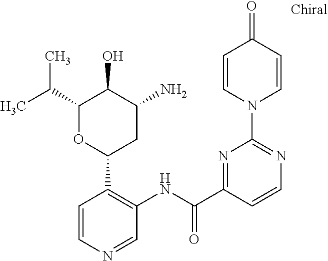 [SMILES: CC(C)[C@H]1O[C@@H](c2ccncc2NC(=O)c2ccnc(-n3ccc(=O)cc3)n2)C[C@@H](N)[C@@H]1O] | 450.5 | 451.1 | 0.42 |
| 305 | 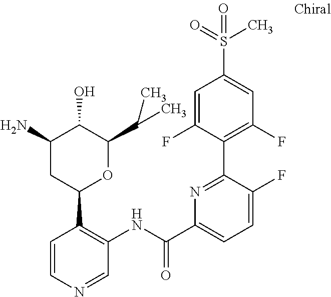 [SMILES: CC(C)[C@H]1O[C@@H](c2ccncc2NC(=O)c2ccc(F)c(-c3c(F)cc(S(C)(=O)=O)cc3F)n2)C[C@@H](N)[C@@H]1O] | 564.6 | 565.0 | 0.59 |
| 306 | 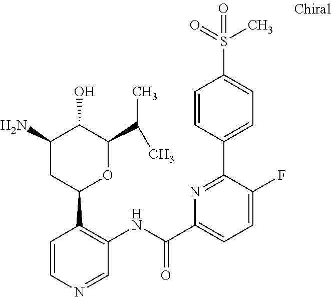 [SMILES: CC(C)[C@H]1O[C@@H](c2ccncc2NC(=O)c2ccc(F)c(-c3ccc(S(C)(=O)=O)cc3)n2)C[C@@H](N)[C@@H]1O] | 528.2 | 529.0 | 0.57 |
| 307 | 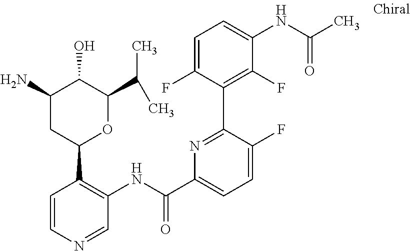 [SMILES: CC(=O)Nc1ccc(F)c(-c2nc(C(=O)Nc3cnccc3[C@H]3C[C@@H](N)[C@H](O)[C@@H](C(C)C)O3)ccc2F)c1F] | 543.2 | 544.0 | 0.58 |
| 308 | 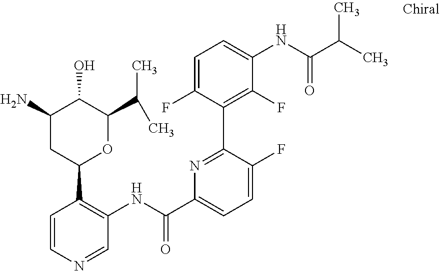 [SMILES: CC(C)C(=O)Nc1ccc(F)c(-c2nc(C(=O)Nc3cnccc3[C@H]3C[C@@H](N)[C@H](O)[C@@H](C(C)C)O3)ccc2F)c1F] | 571.2 | 572.2 | 0.64 |
| 309 | 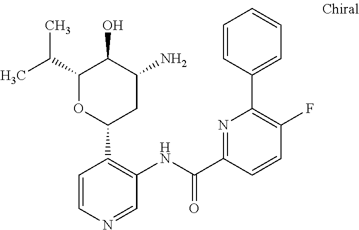 [SMILES: CC(C)[C@H]1O[C@@H](c2ccncc2NC(=O)c2ccc(F)c(-c3ccccc3)n2)C[C@@H](N)[C@@H]1O] | 450.5 | 451.0 | 0.64 |
| 310 | 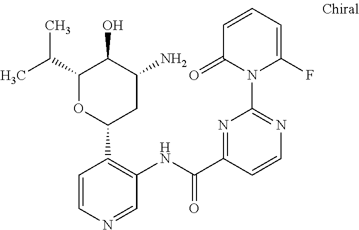 [SMILES: CC(C)[C@H]1O[C@@H](c2ccncc2NC(=O)c2ccnc(-n3c(F)cccc3=O)n2)C[C@@H](N)[C@@H]1O] | 468.5 | 469.1 | 0.55 |
| 311 | 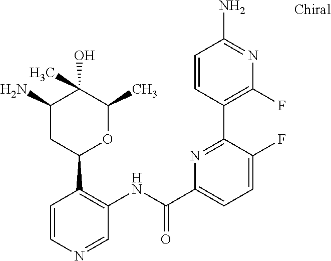 [SMILES: C[C@H]1O[C@@H](c2ccncc2NC(=O)c2ccc(F)c(-c3ccc(N)nc3F)n2)C[C@@H](N)[C@]1(C)O] | 470.5 | 471.0 | 0.49 |
| 312 | 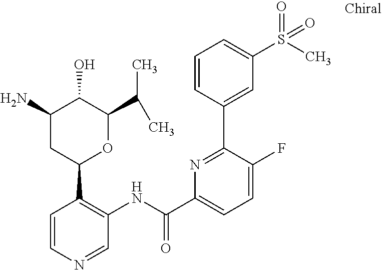 [SMILES: CC(C)[C@H]1O[C@@H](c2ccncc2NC(=O)c2ccc(F)c(-c3cccc(S(C)(=O)=O)c3)n2)C[C@@H](N)[C@@H]1O] | 528.2 | 529.0 | 0.55 |
| 313 | 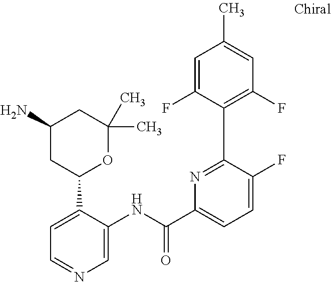 [SMILES: Cc1cc(F)c(-c2nc(C(=O)Nc3cnccc3[C@@H]3C[C@@H](N)CC(C)(C)O3)ccc2F)c(F)c1] | 470.5 | 0.7 | 471.00 |
| 314 | 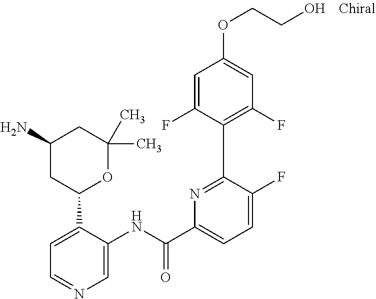 [SMILES: CC1(C)C[C@H](N)C[C@@H](c2ccncc2NC(=O)c2ccc(F)c(-c3c(F)cc(OCCO)cc3F)n2)O1] | 516.5 | 0.6 | 517.10 |
| 315 | 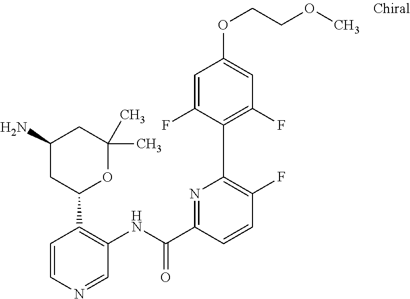 [SMILES: COCCOc1cc(F)c(-c2nc(C(=O)Nc3cnccc3[C@@H]3C[C@@H](N)CC(C)(C)O3)ccc2F)c(F)c1] | 530.5 | 0.6 | 531.10 |
| 316 | 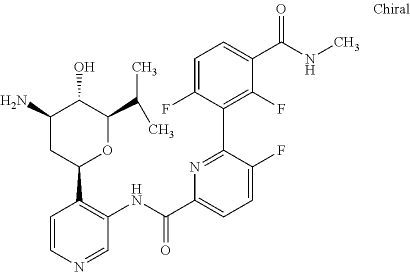 [SMILES: CNC(=O)c1ccc(F)c(-c2nc(C(=O)Nc3cnccc3[C@H]3C[C@@H](N)[C@H](O)[C@@H](C(C)C)O3)ccc2F)c1F] | 543.2 | 544.0 | 0.58 |
| 317 | 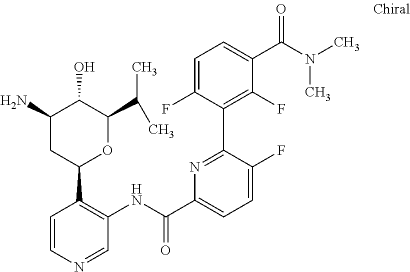 [SMILES: CC(C)[C@H]1O[C@@H](c2ccncc2NC(=O)c2ccc(F)c(-c3c(F)ccc(C(=O)N(C)C)c3F)n2)C[C@@H](N)[C@@H]1O] | 557.2 | 558.0 | 0.61 |
| 318 | 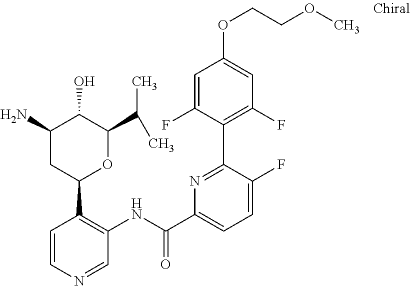 [SMILES: COCCOc1cc(F)c(-c2nc(C(=O)Nc3cnccc3[C@H]3C[C@@H](N)[C@H](O)[C@@H](C(C)C)O3)ccc2F)c(F)c1] | 560.2 | 561.0 | 0.68 |
| 319 | 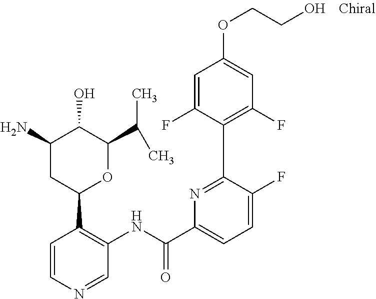 [SMILES: CC(C)[C@H]1O[C@@H](c2ccncc2NC(=O)c2ccc(F)c(-c3c(F)cc(OCCO)cc3F)n2)C[C@@H](N)[C@@H]1O] | 546.5 | 547.0 | 0.61 |
| 320 | 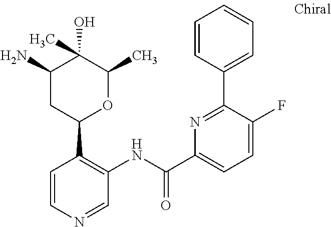 [SMILES: C[C@H]1O[C@@H](c2ccncc2NC(=O)c2ccc(F)c(-c3ccccc3)n2)C[C@@H](N)[C@]1(C)O] | 436.5 | 437.1 | 0.58 |
| 321 | 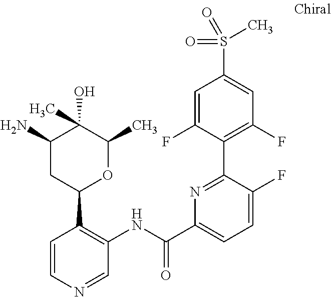 [SMILES: C[C@H]1O[C@@H](c2ccncc2NC(=O)c2ccc(F)c(-c3c(F)cc(S(C)(=O)=O)cc3F)n2)C[C@@H](N)[C@]1(C)O] | 514.6 | 515.1 | 0.51 |
| 322 | 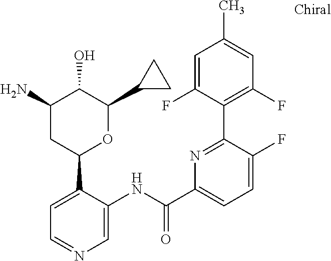 [SMILES: Cc1cc(F)c(-c2nc(C(=O)Nc3cnccc3[C@H]3C[C@@H](N)[C@H](O)[C@@H](C4CC4)O3)ccc2F)c(F)c1] | 498.5 | 499.1 | 0.64 |
| 323 | 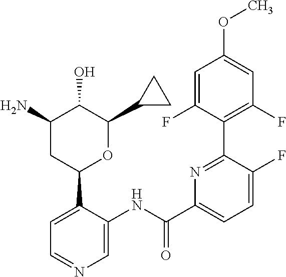 [SMILES: COc1cc(F)c(-c2nc(C(=O)Nc3cnccc3[C@H]3C[C@@H](N)[C@H](O)[C@@H](C4CC4)O3)ccc2F)c(F)c1] | 514.5 | 515.0 | 0.62 |
| 324 | 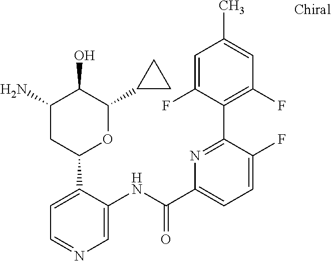 [SMILES: Cc1cc(F)c(-c2nc(C(=O)Nc3cnccc3[C@@H]3C[C@H](N)[C@@H](O)[C@H](C4CC4)O3)ccc2F)c(F)c1] | 498.5 | 499.1 | 0.63 |
| 325 | 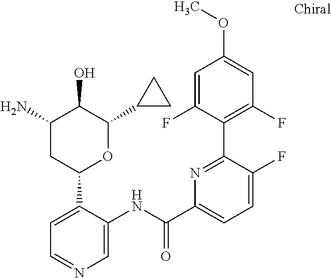 [SMILES: COc1cc(F)c(-c2nc(C(=O)Nc3cnccc3[C@@H]3C[C@H](N)[C@@H](O)[C@H](C4CC4)O3)ccc2F)c(F)c1] | 514.5 | 515.0 | 0.63 |
| 326 | 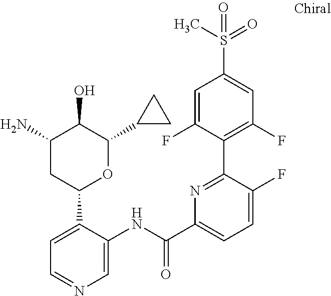 [SMILES: CS(=O)(=O)c1cc(F)c(-c2nc(C(=O)Nc3cnccc3[C@@H]3C[C@H](N)[C@@H](O)[C@H](C4CC4)O3)ccc2F)c(F)c1] | 562.6 | 563.0 | 0.57 |
| 327 | 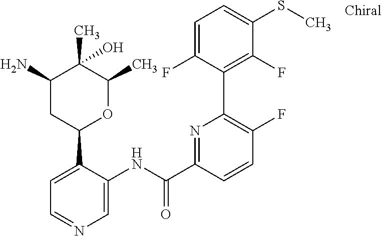 [SMILES: CSc1ccc(F)c(-c2nc(C(=O)Nc3cnccc3[C@H]3C[C@@H](N)[C@](C)(O)[C@@H](C)O3)ccc2F)c1F] | 518.6 | 519.0 | 0.63 |
| 328 | 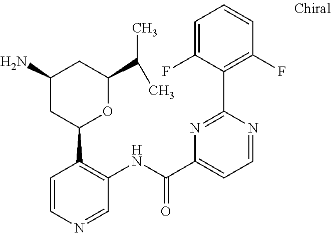 [SMILES: CC(C)[C@@H]1C[C@H](N)C[C@H](c2ccncc2NC(=O)c2ccnc(-c3c(F)cccc3F)n2)O1] | 453.5 | 454.0 | 0.59 |
| 329 | 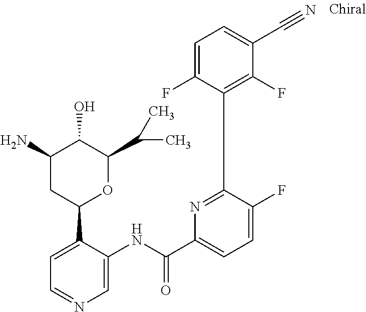 [SMILES: CC(C)[C@H]1O[C@@H](c2ccncc2NC(=O)c2ccc(F)c(-c3c(F)ccc(C#N)c3F)n2)C[C@@H](N)[C@@H]1O] | 511.5 | 512.0 | 0.60 |
| 330 | 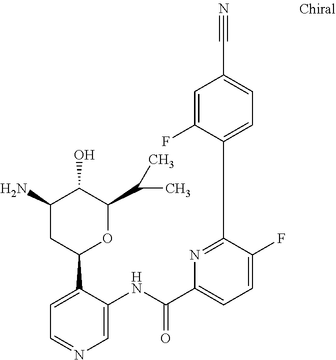 [SMILES: CC(C)[C@H]1O[C@@H](c2ccncc2NC(=O)c2ccc(F)c(-c3ccc(C#N)cc3F)n2)C[C@@H](N)[C@@H]1O] | 493.5 | 494.0 | 0.61 |
| 331 | 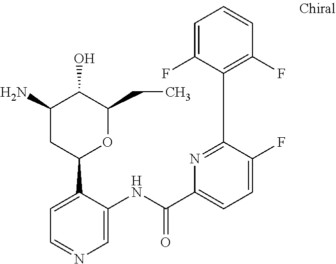 [SMILES: CC[C@H]1O[C@@H](c2ccncc2NC(=O)c2ccc(F)c(-c3c(F)cccc3F)n2)C[C@@H](N)[C@@H]1O] | 472.5 | 473.0 | 0.59 |
| 332 | 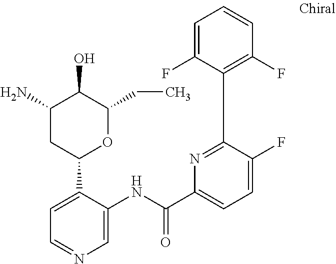 [SMILES: CC[C@@H]1O[C@H](c2ccncc2NC(=O)c2ccc(F)c(-c3c(F)cccc3F)n2)C[C@H](N)[C@H]1O] | 472.5 | 473.0 | 0.59 |
| 333 | 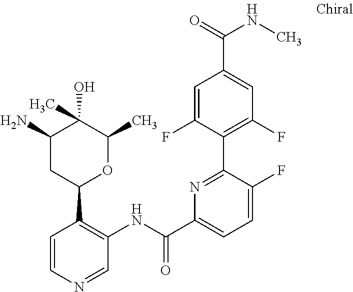 [SMILES: CNC(=O)c1cc(F)c(-c2nc(C(=O)Nc3cnccc3[C@H]3C[C@@H](N)[C@](C)(O)[C@@H](C)O3)ccc2F)c(F)c1] | 529.5 | 530.0 | 0.55 |
| 334 | 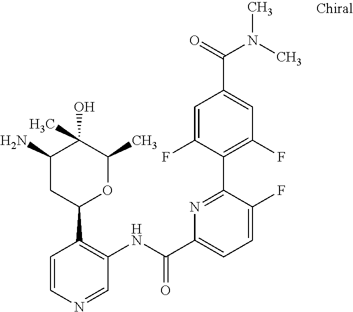 [SMILES: C[C@H]1O[C@@H](c2ccncc2NC(=O)c2ccc(F)c(-c3c(F)cc(C(=O)N(C)C)cc3F)n2)C[C@@H](N)[C@]1(C)O] | 543.5 | 543.9 | 0.57 |
| 335 | 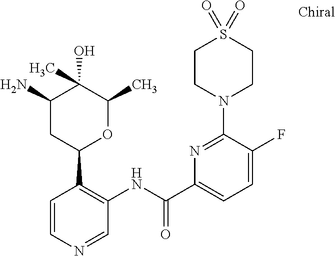 [SMILES: C[C@H]1O[C@@H](c2ccncc2NC(=O)c2ccc(F)c(N3CCS(=O)(=O)CC3)n2)C[C@@H](N)[C@]1(C)O] | 493.6 | 493.9 | 0.41 |
| 336 | 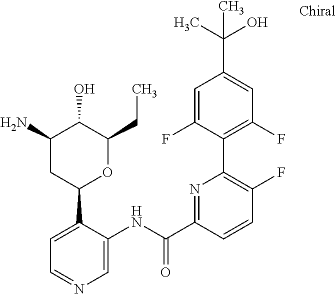 [SMILES: CC[C@H]1O[C@@H](c2ccncc2NC(=O)c2ccc(F)c(-c3c(F)cc(C(C)(C)O)cc3F)n2)C[C@@H](N)[C@@H]1O] | 530.5 | 531.1 | 0.61 |
| 337 | 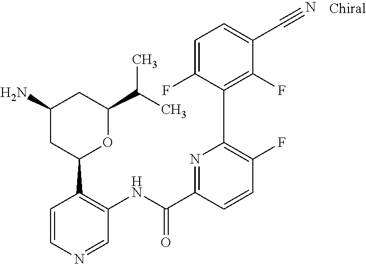 [SMILES: CC(C)[C@@H]1C[C@H](N)C[C@H](c2ccncc2NC(=O)c2ccc(F)c(-c3c(F)ccc(C#N)c3F)n2)O1] | 495.5 | 496.1 | 0.64 |
| 338 | 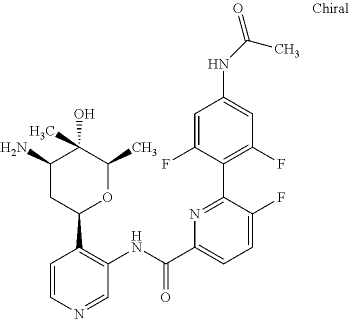 [SMILES: CC(=O)Nc1cc(F)c(-c2nc(C(=O)Nc3cnccc3[C@H]3C[C@@H](N)[C@](C)(O)[C@@H](C)O3)ccc2F)c(F)c1] | 529.5 | 530.2 | 0.56 |
| 339 | 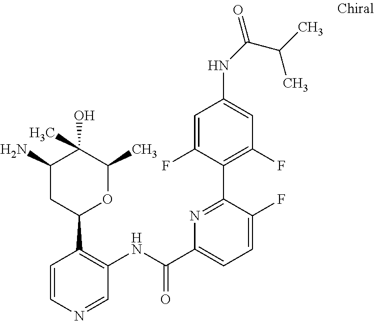 [SMILES: CC(C)C(=O)Nc1cc(F)c(-c2nc(C(=O)Nc3cnccc3[C@H]3C[C@@H](N)[C@](C)(O)[C@@H](C)O3)ccc2F)c(F)c1] | 557.6 | 558.0 | 0.64 |
| 340 | 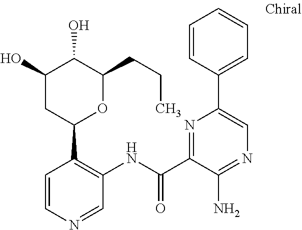 [SMILES: CCC[C@H]1O[C@@H](c2ccncc2NC(=O)c2nc(-c3ccccc3)cnc2N)C[C@@H](O)[C@@H]1O] | 449.5 | 450.0 | 0.64 |
| 341 | 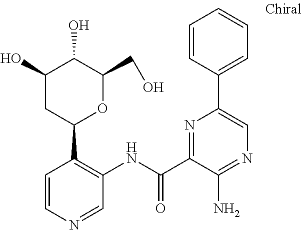 [SMILES: Nc1ncc(-c2ccccc2)nc1C(=O)Nc1cnccc1[C@H]1C[C@@H](O)[C@H](O)[C@@H](CO)O1] | 437.5 | 438.0 | 0.51 |
| 342 | 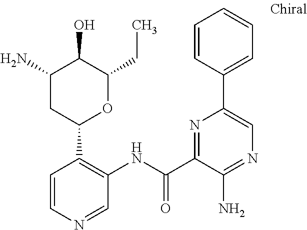 [SMILES: CC[C@@H]1O[C@H](c2ccncc2NC(=O)c2nc(-c3ccccc3)cnc2N)C[C@H](N)[C@H]1O] | 434.5 | 435.0 | 0.56 |
| 343 | 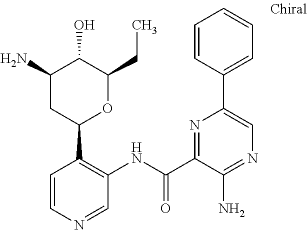 [SMILES: CC[C@H]1O[C@@H](c2ccncc2NC(=O)c2nc(-c3ccccc3)cnc2N)C[C@@H](N)[C@@H]1O] | 434.5 | 435.1 | 0.57 |
| 344 | 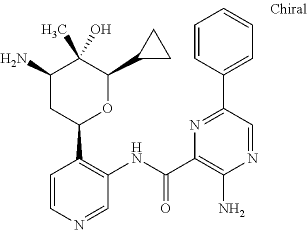 [SMILES: C[C@]1(O)[C@H](N)C[C@H](c2ccncc2NC(=O)c2nc(-c3ccccc3)cnc2N)O[C@@H]1C1CC1] | 460.5 | 461.1 | 0.60 |
| 345 | 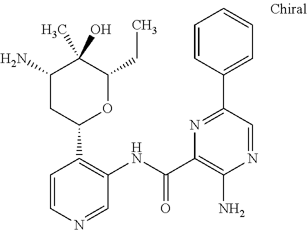 [SMILES: CC[C@@H]1O[C@H](c2ccncc2NC(=O)c2nc(-c3ccccc3)cnc2N)C[C@H](N)[C@@]1(C)O] | 448.5 | 449.2 | 0.59 |
| 346 | 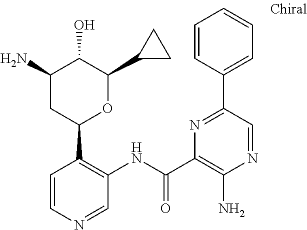 [SMILES: Nc1ncc(-c2ccccc2)nc1C(=O)Nc1cnccc1[C@H]1C[C@@H](N)[C@H](O)[C@@H](C2CC2)O1] | 446.5 | 447.2 | 0.60 |
| 347 | 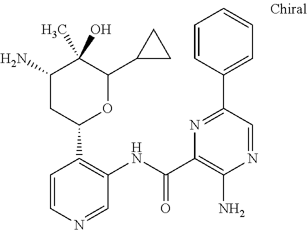 [SMILES: C[C@]1(O)C(C2CC2)O[C@H](c2ccncc2NC(=O)c2nc(-c3ccccc3)cnc2N)C[C@@H]1N] | 460.5 | 461.1 | 0.59 |
| 348 | 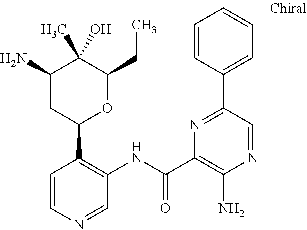 [SMILES: CC[C@H]1O[C@@H](c2ccncc2NC(=O)c2nc(-c3ccccc3)cnc2N)C[C@@H](N)[C@]1(C)O] | 448.5 | 449.2 | 0.63 |
| 349 | 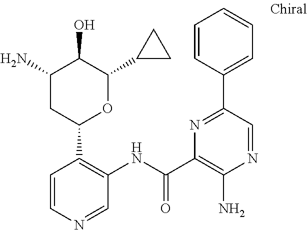 [SMILES: Nc1ncc(-c2ccccc2)nc1C(=O)Nc1cnccc1[C@@H]1C[C@H](N)[C@@H](O)[C@H](C2CC2)O1] | 446.5 | 447.1 | 0.59 |
| 350 | 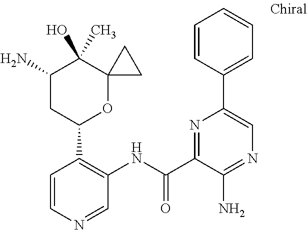 [SMILES: C[C@@]1(O)[C@@H](N)C[C@@H](c2ccncc2NC(=O)c2nc(-c3ccccc3)cnc2N)OC12CC2] | 446.5 | 447.1 | 0.57 |
| 351 | 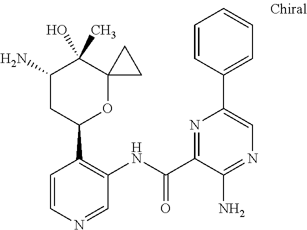 [SMILES: C[C@]1(O)[C@@H](N)C[C@H](c2ccncc2NC(=O)c2nc(-c3ccccc3)cnc2N)OC12CC2] | 446.5 | 447.2 | 0.57 |
| 352 | 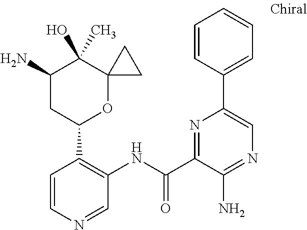 [SMILES: C[C@@]1(O)[C@H](N)C[C@@H](c2ccncc2NC(=O)c2nc(-c3ccccc3)cnc2N)OC12CC2] | 446.5 | 447.1 | 0.57 |
| 353 | 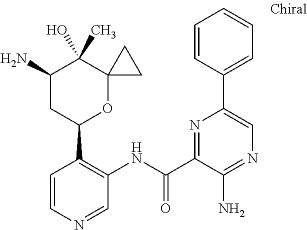 [SMILES: C[C@]1(O)[C@H](N)C[C@H](c2ccncc2NC(=O)c2nc(-c3ccccc3)cnc2N)OC12CC2] | 446.5 | 447.1 | 0.57 |
| 354 | 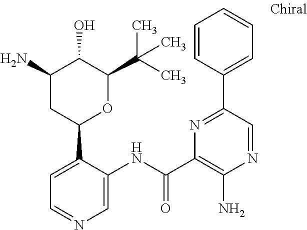 [SMILES: CC(C)(C)[C@H]1O[C@@H](c2ccncc2NC(=O)c2nc(-c3ccccc3)cnc2N)C[C@@H](N)[C@@H]1O] | 462.6 | 463.1 | 0.68 |
| 355 | 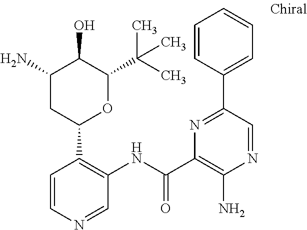 [SMILES: CC(C)(C)[C@@H]1O[C@H](c2ccncc2NC(=O)c2nc(-c3ccccc3)cnc2N)C[C@H](N)[C@H]1O] | 462.6 | 463.1 | 0.67 |
| 356 | 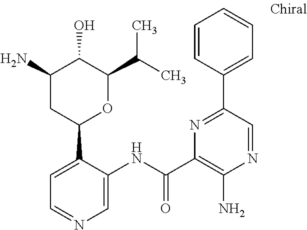 [SMILES: CC(C)[C@H]1O[C@@H](c2ccncc2NC(=O)c2nc(-c3ccccc3)cnc2N)C[C@@H](N)[C@@H]1O] | 448.5 | 449.2 | 0.62 |

Table 2 provides chemical names for all the compounds in Table 1 and ¹H NMR data for some of the compounds in Table 1.

TABLE 2

| Ex # | IUPAC Name | ¹H-NMR |
|---|---|---|
| 1 | N-(4-((2R,4R,5S,6S)-6-(chloromethyl)-4,5- | |
| | dihydroxytetrahydro-2H-pyran-2-yl)pyridin-3- | |
| | yl)-6-(3-cyano-2,6-difluorophenyl)-5- | |
| | fluoropicolinamide | |
| 2 | 6-(3-cyano-2,6-difluorophenyl)-N-(4- | |
| | ((2R,4R,5S,6R)-6-ethyl-4,5- | |
| | dihydroxytetrahydro-2H-pyran-2-yl)pyridin-3- | |
| | yl)-5-fluoropicolinamide | |
| 3 | 3-amino-N-(4-((2R,4R,5S,6R)-5-ethyl-4,5- | |
| | dihydroxy-6-methyltetrahydro-2H-pyran-2- | |
| | yl)pyridin-3-yl)-6-(thiazol-2-yl)picolinamide | |
| 4 | 3-amino-N-(4-((2R,3S,4R)-2-cyano-3,4- | |
| | dihydroxy-3,4-dihydro-2H-pyran-6-yl)pyridin- | |
| | 3-yl)-6-(2,6-difluorophenyl)-5- | |
| | fluoropicolinamide | |
| 5 | 3-amino-N-(4-((5S,7S)-7-hydroxy-8- | |
| | methylene-4-oxaspiro[2.5]octan-5-yl)pyridin- | |
| | 3-yl)-6-phenylpyrazine-2-carboxamide | |
| 6 | 5-amino-2-(2,6-difluorophenyl)-N-(4- | |
| | ((5S,7S)-7-hydroxy-8-methylene-4- | |
| | oxaspiro[2.5]octan-5-yl)pyridin-3- | |
| | yl)pyrimidine-4-carboxamide | |
| 7 | 6-(2,6-difluorophenyl)-5-fluoro-N-(4-((5S,7S)- | |
| | 7-hydroxy-8-methylene-4-oxaspiro[2.5]octan- | |
| | 5-yl)pyridin-3-yl)picolinamide | |
| 8 | 5-amino-2-(2,6-difluorophenyl)-N-(4- | |
| | ((5R,7R)-7-hydroxy-8-methylene-4- | |
| | oxaspiro[2.5]octan-5-yl)pyridin-3- | |
| | yl)pyrimidine-4-carboxamide | |
| 9 | 6-(2,6-difluorophenyl)-5-fluoro-N-(4- | |
| | ((5R,7R)-7-hydroxy-8-methylene-4- | |
| | oxaspiro[2.5]octan-5-yl)pyridin-3- | |
| | yl)picolinamide | |
| 10 | 5-amino-2-(2,6-difluorophenyl)-N-(4- | |
| | ((2S,3R,4S)-3,4-dihydroxy-2,3-dimethyl-3,4- | |
| | dihydro-2H-pyran-6-yl)pyridin-3- | |
| | yl)pyrimidine-4-carboxamide | |
| 11 | 5-amino-2-(2,6-difluorophenyl)-N-(4- | |
| | ((2R,3S,4R)-3,4-dihydroxy-2,3-dimethyl-3,4- | |
| | dihydro-2H-pyran-6-yl)pyridin-3- | |
| | yl)pyrimidine-4-carboxamide | |
| 12 | 3-amino-N-(4-((2R,4R,5S,6R)-6-cyano-4,5- | |
| | dihydroxytetrahydro-2H-pyran-2-yl)pyridin-3- | |
| | yl)-6-(2,6-difluorophenyl)-5- | |
| | fluoropicolinamide | |
| 13 | N-(4-((2R,4R,5S,6R)-6-cyano-4,5- | |
| | dihydroxytetrahydro-2H-pyran-2-yl)pyridin-3- | |
| | yl)-6-(2,6-difluorophenyl)-5- | |
| | fluoropicolinamide | |
| 14 | 5-amino-N-(4-((2S,3S,4R)-2-(chloromethyl)- | |
| | 3,4-dihydroxy-3,4-dihydro-2H-pyran-6- | |
| | yl)pyridin-3-yl)-2-(2,6- | |
| | difluorophenyl)pyrimidine-4-carboxamide | |
| 15 | 3-amino-N-(4-((2R,4R,5S,6R)-5-ethyl-4,5- | |
| | dihydroxy-6-methyltetrahydro-2H-pyran-2- | |
| | yl)pyridin-3-yl)-6-phenylpyrazine-2- | |
| | carboxamide | |
| 16 | 5-amino-2-(2,6-difluorophenyl)-N-(4- | 1H-NMR (400 mHz, DMSO- |
| | ((2R,4R,5S,6R)-5-ethyl-4,5-dihydroxy-6- | d6) d 10.60 (s, 1H), 9.24 (s, |
| | methyltetrahydro-2H-pyran-2-yl)pyridin-3- | 1H), 8.70 (s, 1H), 8.50 (d, 1H), |
| | yl)pyrimidine-4-carboxamide | 7.62 (d, 1H), 7.49-7.55 (m, 1H), |
| | | 7.19 (t, 2H), 4.76 (dd, 1H), |
| | | 3.54-3.58 (m, 1H), 3.22 (q, 1H), |
| | | 1.87-1.92 (m, 1H), 1.63 (dd, |
| | | 1H), 1.41-1.49 (m, 1H), |
| | | 1.21-1.28 (m, 1H), 0.71 (t, 3H), |
| | | 0.69 (d, H). |
| 17 | 5-amino-N-(4-((2R,4R,5S,6S)-6- | |
| | (chloromethyl)-4,5-dihydroxytetrahydro-2H- | |
| | pyran-2-yl)pyridin-3-yl)-2-(2,6- | |
| | difluorophenyl)pyrimidine-4-carboxamide | |
| 18 | 5-amino-2-(2,6-difluorophenyl)-N-(4- | 1H-NMR (CD₃OD): d 10.6 (s, |
| | ((2R,4R,5S,6R)-6-ethyl-4,5- | 1H), 9.22 (s, 1H), 8.51 (s, 1H), |
| | dihydroxytetrahydro-2H-pyran-2-yl)pyridin-3- | 8.22 (d, 1H), 7.35-7.46 (m, 1H), |
| | yl)pyrimidine-4-carboxamide | 7.30 (d, 1H), 6.99-7.06 (m, 2H), |
| | | 4.55 (dd, 1H), 3.50 (m, 1H), |
| | | 3.00 (m, 1H), 2.83 (t, 1H), |
| | | 2.01 (ddd, 1H), 1.62-1.75 (m, 1H), |
| | | 1.38 (ddd, 1H), 1.0-1.1 (m, 2H), |
| | | 0.87-1.0 (m, 1H), 0.62 (t, 3H) |
| 19 | 6-(2,6-difluorophenyl)-N-(4-((2S,4S,5R,6S)- | |
| | 4,5-dihydroxy-5-(hydroxymethyl)-6- | |
| | methyltetrahydro-2H-pyran-2-yl)pyridin-3-yl)- | |
| | 5-fluoropicolinamide | |
| 20 | 6-(2,6-difluorophenyl)-N-(4-((2R,4R,5S,6R)- | |
| | 4,5-dihydroxy-5-(hydroxymethyl)-6- | |
| | methyltetrahydro-2H-pyran-2-yl)pyridin-3-yl)- | |
| | 5-fluoropicolinamide | |
| 21 | N-(4-((2R,3S,4R)-2-cyano-3,4-dihydroxy-3,4- | |
| | dihydro-2H-pyran-6-yl)pyridin-3-yl)-6-(2,6- | |
| | difluorophenyl)-5-fluoropicolinamide | |
| 22 | 5-amino-N-(4-((2R,3S,4R)-3,4-dihydroxy-2- | |
| | vinyl-3,4-dihydro-2H-pyran-6-yl)pyridin-3- | |
| | yl)-2-phenylpyrimidine-4-carboxamide | |
| 23 | 2-(2,6-difluorophenyl)-N-(4-((2R,3S,4R)-3,4- | |
| | dihydroxy-2-vinyl-3,4-dihydro-2H-pyran-6- | |
| | yl)pyridin-3-yl)pyrimidine-4-carboxamide | |
| 24 | 3-amino-N-(4-((2R,3S,4R)-3,4-dihydroxy-2- | |
| | vinyl-3,4-dihydro-2H-pyran-6-yl)pyridin-3- | |
| | yl)-5-fluoro-6-(2-fluorophenyl)picolinamide | |
| 25 | 3-amino-6-(2,6-difluorophenyl)-N-(4- | |
| | ((2R,3S,4R)-3,4-dihydroxy-2-vinyl-3,4- | |
| | dihydro-2H-pyran-6-yl)pyridin-3-yl)-5- | |
| | fluoropicolinamide | |
| 26 | 5-amino-N-(4-((2R,4R,5S,6R)-5-ethyl-4,5- | |
| | dihydroxy-6-methyltetrahydro-2H-pyran-2- | |
| | yl)pyridin-3-yl)-2-phenylpyrimidine-4- | |
| | carboxamide | |
| 27 | 3-amino-6-(2,6-difluorophenyl)-N-(4- | 1H-NMR [400 mHz, DMSOd- |
| | ((2R,4R,5S,6R)-5-ethyl-4,5-dihydroxy-6- | 6, d 10.36 (s, 1H), 9.22 (2, H), |
| | methyltetrahydro-2H-pyran-2-yl)pyridin-3-yl)- | 8.48 (d, 1H), 7.62 (d, 1H), |
| | 5-fluoropicolinamide | 7.52-7.58 (m, 1H), 7.26 (d, 1H), |
| | | 7.22 (t, 2H), 4.72 (dd, 1H), 3.97 (bs, |
| | | 2H), 3.55 (dd, 1H), 3.18 (q, |
| | | 1H), 1.89 (ddd, 1H), 1.63 (q, |
| | | 1H), 1.41-1.48 (m, 1H), |
| | | 1.19-1.28 (m, 1H), 0.73 (t, 3H), |
| | | 0.62 (d, 3H). |
| 28 | N-(4-((2S,3S,4R)-2-(chloromethyl)-3,4- | |
| | dihydroxy-3,4-dihydro-2H-pyran-6-yl)pyridin- | |
| | 3-yl)-2-(2,6-difluorophenyl)pyrimidine-4- | |
| | carboxamide | |
| 29 | 3-amino-N-(4-((2S,3S,4R)-2-(chloromethyl)- | |
| | 3,4-dihydroxy-3,4-dihydro-2H-pyran-6- | |
| | yl)pyridin-3-yl)-5-fluoro-6-(2- | |
| | fluorophenyl)picolinamide | |
| 30 | 3-amino-N-(4-((2S,3S,4R)-2-(chloromethyl)- | |
| | 3,4-dihydroxy-3,4-dihydro-2H-pyran-6- | |
| | yl)pyridin-3-yl)-6-(2,6-difluorophenyl)-5- | |
| | fluoropicolinamide | |
| 31 | N-(4-((2S,3S,4R)-2-(chloromethyl)-3,4- | |
| | dihydroxy-3,4-dihydro-2H-pyran-6-yl)pyridin- | |
| | 3-yl)-6-(2,6-difluorophenyl)-5- | |
| | fluoropicolinamide | |
| 32 | 5-amino-N-(4-((2R,4R,5S,6R)-4,5-dihydroxy- | |
| | 5,6-dimethyltetrahydro-2H-pyran-2- | |
| | yl)pyridin-3-yl)-2-phenylpyrimidine-4- | |
| | carboxamide | |
| 33 | 3-amino-6-(2,6-difluorophenyl)-N-(4- | |
| | ((2R,4R,5S,6R)-4,5-dihydroxy-5,6- | |
| | dimethyltetrahydro-2H-pyran-2-yl)pyridin-3- | |
| | yl)-5-fluoropicolinamide | |
| 34 | 2-(2,6-difluorophenyl)-N-(4-((2R,4R,5S,6R)- | |
| | 6-ethyl-4,5-dihydroxytetrahydro-2H-pyran-2- | |
| | yl)pyridin-3-yl)pyrimidine-4-carboxamide | |
| 35 | 3-amino-N-(4-((2R,4R,5S,6S)-6- | |
| | (chloromethyl)-4,5-dihydroxytetrahydro-2H- | |
| | pyran-2-yl)pyridin-3-yl)-6-(2,6- | |
| | difluorophenyl)-5-fluoropicolinamide | |
| 36 | 3-amino-N-(4-((2R,4R,5S,6S)-6- | |
| | (chloromethyl)-4,5-dihydroxytetrahydro-2H- | |
| | pyran-2-yl)pyridin-3-yl)-6-phenylpyrazine-2- | |
| | carboxamide | |
| 37 | N-(4-((2R,4R,5S,6S)-6-(chloromethyl)-4,5- | |
| | dihydroxytetrahydro-2H-pyran-2-yl)pyridin-3- | |
| | yl)-2-(2,6-difluorophenyl)pyrimidine-4- | |
| | carboxamide | |
| 38 | 3-amino-N-(4-((2R,4R,5S,6S)-6- | |
| | (chloromethyl)-4,5-dihydroxytetrahydro-2H- | |
| | pyran-2-yl)pyridin-3-yl)-5-fluoro-6-(2- | |
| | fluorophenyl)picolinamide | |
| 39 | 5-amino-N-(4-((2R,4R,5S,6R)-6-ethyl-4,5- | 1H NMR (400 MHz, DMSO-d6) δ |
| | dihydroxytetrahydro-2H-pyran-2-yl)pyridin-3- | ppm 0.82 (t, 3 H) 1.29-1.47 (m, 2 |
| | yl)-2-phenylpyrimidine-4-carboxamide | H) 1.67-1.83 (m, 1 H) 2.12 (dd, 1 |
| | | H) 2.87 (t, 1 H) 3.02-3.16 (m, 1 |
| | | H) 3.37-3.48 (m, 1 H) 4.76 (d, 1 |
| | | H) 4.89 (br. s., 2 H) 7.02 (br. s., 2 |
| | | H) 7.36-7.54 (m, 4 H) 8.39 (d, 2 |
| | | H) 8.45 (d, 1 H) 8.64 (s, 1 H) |
| | | 8.81 (s, 1 H) 10.41 (s, 1 H) |
| 40 | 5-amino-2-(2,6-difluorophenyl)-N-(4- | |
| | ((2R,4R,5S,6R)-6-ethyl-4,5- | |
| | dihydroxytetrahydro-2H-pyran-2-yl)pyridin-3- | |
| | yl)thiazole-4-carboxamide | |
| 41 | 2-(2,6-difluorophenyl)-N-(4-((2R,4R,5S,6R)- | |
| | 6-ethyl-4,5-dihydroxytetrahydro-2H-pyran-2- | |
| | yl)pyridin-3-yl)thiazole-4-carboxamide | |
| 42 | 3-amino-N-(4-((2R,4R,5S,6R)-6-ethyl-4,5- | |
| | dihydroxytetrahydro-2H-pyran-2-yl)pyridin-3- | |
| | yl)-5-fluoro-6-(2-fluorophenyl)picolinamide | |
| 43 | 3-amino-6-(2,6-difluorophenyl)-N-(4- | |
| | ((2R,4R,5S,6R)-6-ethyl-4,5- | |
| | dihydroxytetrahydro-2H-pyran-2-yl)pyridin-3- | |
| | yl)-5-fluoropicolinamide | |
| 44 | 6-(2,6-difluorophenyl)-N-(4-((2R,3S,4R)-3,4- | |
| | dihydroxy-2-vinyl-3,4-dihydro-2H-pyran-6- | |
| | yl)pyridin-3-yl)-5-fluoropicolinamide | |
| 45 | 5-cyano-N-(4-((2R,5S,6R)-6-(cyanomethyl)-5- | |
| | hydroxytetrahydro-2H-pyran-2-yl)pyridin-3- | |
| | yl)-6-(2,6-difluorophenyl)picolinamide | |
| 46 | N-(4-((2R,5S,6R)-6-(cyanomethyl)-5- | |
| | hydroxytetrahydro-2H-pyran-2-yl)pyridin-3- | |
| | yl)-6-(2,6-difluorophenyl)-5- | |
| | fluoropicolinamide | |
| 47 | 5-cyano-N-(4-((2R,4R,5S,6R)-6- | |
| | (cyanomethyl)-4,5-dihydroxytetrahydro-2H- | |
| | pyran-2-yl)pyridin-3-yl)-6-(2,6- | |
| | difluorophenyl)picolinamide | |
| 48 | 6-(2,6-difluorophenyl)-5-fluoro-N-(4- | |
| | ((2R,4R)-4-hydroxy-2-(trifluoromethyl)-3,4- | |
| | dihydro-2H-pyran-2-yl)pyridin-3- | |
| | yl)picolinamide | |
| 49 | 6-(2,6-difluorophenyl)-5-fluoro-N-(4-((2S,4S)- | |
| | 4-hydroxy-2-(trifluoromethyl)-3,4-dihydro- | |
| | 2H-pyran-2-yl)pyridin-3-yl)picolinamide | |
| 50 | 6-(2,6-difluorophenyl)-5-fluoro-N-(4- | |
| | ((2S,4R)-4-hydroxy-2- | |
| | (trifluoromethyl)tetrahydro-2H-pyran-2- | |
| | yl)pyridin-3-yl)picolinamide | |
| 51 | (R)-6-(2,6-difluorophenyl)-5-fluoro-N-(4-(2- | |
| | (trifluoromethyl)tetrahydro-2H-pyran-2- | |
| | yl)pyridin-3-yl)picolinamide | |
| 52 | (S)-6-(2,6-difluorophenyl)-5-fluoro-N-(4-(2- | |
| | (trifluoromethyl)tetrahydro-2H-pyran-2- | |
| | yl)pyridin-3-yl)picolinamide | |
| 53 | 6-(2,6-difluorophenyl)-5-fluoro-N-(4- | |
| | ((2R,4S)-4-hydroxy-2- | |
| | (trifluoromethyl)tetrahydro-2H-pyran-2- | |
| | yl)pyridin-3-yl)picolinamide | |
| 54 | 6-(2,6-difluorophenyl)-N-(4-((2R,4R,5S,6R)- | |
| | 6-ethyl-4,5-dihydroxy-2,3- | |
| | didueterotetrahydro-2H-pyran-2-yl)pyridin-3- | |
| | yl)-5-fluoropicolinamide | |
| 55 | N-(4-((2R,4S,5R,6S)-4-chloro-6- | |
| | (chloromethyl)-5-hydroxytetrahydro-2H- | |
| | pyran-2-yl)pyridin-3-yl)-6-(2,6- | |
| | difluorophenyl)-5-fluoropicolinamide | |
| 56 | N-(4-((2R,4R,5S,6S)-6-(chloromethyl)-4,5- | |
| | dihydroxytetrahydro-2H-pyran-2-yl)pyridin-3- | |
| | yl)-6-(2,6-difluorophenyl)-5- | |
| | fluoropicolinamide | |
| 57 | 6-(2,6-difluorophenyl)-N-(4-((2R,4R,5S,6R)- | |
| | 4,5-dihydroxy-6-(methoxymethyl)tetrahydro- | |
| | 2H-pyran-2-yl)pyridin-3-yl)-5- | |
| | fluoropicolinamide | |
| 58 | 3-amino-N-(4-((2S,3R,4S,5S,6R)-3-fluoro- | |
| | 4,5-dihydroxy-5,6-dimethyltetrahydro-2H- | |
| | pyran-2-yl)pyridin-3-yl)-6-phenylpyrazine-2- | |
| | carboxamide | |
| 59 | 6-(2,6-difluorophenyl)-5-fluoro-N-(4- | |
| | ((5R,7R)-7-hydroxy-4-oxaspiro[2.5]octan-5- | |
| | yl)pyridin-3-yl)picolinamide | |
| 60 | 6-(2,6-difluorophenyl)-5-fluoro-N-(4-((5S,7S)- | |
| | 7-hydroxy-4-oxaspiro[2.5]octan-5-yl)pyridin- | |
| | 3-yl)picolinamide | |
| 61 | 2-(2,6-difluorophenyl)-N-(4-((5R,7R)-7- | |
| | hydroxy-4-oxaspiro[2.5]octan-5-yl)pyridin-3- | |
| | yl)thiazole-4-carboxamide | |
| 62 | 2-(2,6-difluorophenyl)-N-(4-((5S,7S)-7- | |
| | hydroxy-4-oxaspiro[2.5]octan-5-yl)pyridin-3- | |
| | yl)thiazole-4-carboxamide | |
| 63 | 3-amino-N-(4-((2R,3S,4R,5R,6S)-3-fluoro- | |
| | 4,5-dihydroxy-5,6-dimethyltetrahydro-2H- | |
| | pyran-2-yl)pyridin-3-yl)-6-phenylpyrazine-2- | |
| | carboxamide | |
| 64 | 6-(2,6-difluorophenyl)-N-(4- | |
| | ((2S,3R,4S,5S,6S)-4,5-dihydroxy-3,6- | |
| | dimethyltetrahydro-2H-pyran-2-yl)pyridin-3- | |
| | yl)-5-fluoropicolinamide | |
| 65 | 6-(2,6-difluorophenyl)-N-(4- | |
| | ((2R,3S,4R,5R,6R)-4,5-dihydroxy-3,6- | |
| | dimethyltetrahydro-2H-pyran-2-yl)pyridin-3- | |
| | yl)-5-fluoropicolinamide | |
| 66 | 6-(2,6-difluorophenyl)-N-(4- | |
| | ((2S,3R,4S,5R,6S)-4,5-dihydroxy-3,6- | |
| | dimethyltetrahydro-2H-pyran-2-yl)pyridin-3- | |
| | yl)-5-fluoropicolinamide | |
| 67 | 6-(2,6-difluorophenyl)-N-(4- | |
| | ((2R,3S,4R,5S,6R)-4,5-dihydroxy-3,6- | |
| | dimethyltetrahydro-2H-pyran-2-yl)pyridin-3- | |
| | yl)-5-fluoropicolinamide | |
| 68 | 2-(2,6-difluorophenyl)-N-(4- | |
| | ((2S,3R,4S,5S,6R)-3-fluoro-4,5-dihydroxy- | |
| | 5,6-dimethyltetrahydro-2H-pyran-2- | |
| | yl)pyridin-3-yl)thiazole-4-carboxamide | |
| 69 | 6-(2,6-difluorophenyl)-5-fluoro-N-(4- | 1H NMR (400 MHz, CDCl3) |
| | ((2S,3R,4S,5S,6R)-3-fluoro-4,5-dihydroxy- | d: 10.31 (s, 1H), 9.51 (s, 1H), |
| | 5,6-dimethyltetrahydro-2H-pyran-2- | 8.46-8.49 (m, 2H), 7.67 (dd, |
| | yl)pyridin-3-yl)picolinamide | 1H), 7.46-7.52 (m, 1H), |
| | | 7.30 (d,1H), 7.08 (dd, 1H), 4.56 (dd, |
| | | 1H), 4.40 (ddd, 1H), |
| | | 3.82 (dd,1H), 3.54 (q,1H), 1.04 (s, |
| | | 3H), 0.89 (d, 3H). |
| 70 | 2-(2,6-difluorophenyl)-N-(4- | |
| | ((2R,3S,4R,5R,6S)-3-fluoro-4,5-dihydroxy- | |
| | 5,6-dimethyltetrahydro-2H-pyran-2- | |
| | yl)pyridin-3-yl)thiazole-4-carboxamide | |
| 71 | 6-(2,6-difluorophenyl)-5-fluoro-N-(4- | 1H NMR (400 MHz, DMSO- |
| | ((2R,3S,4R,5R,6S)-3-fluoro-4,5-dihydroxy- | d6) d: ppm 0.65-0.70 (m, 6 H) |
| | 5,6-dimethyltetrahydro-2H-pyran-2- | 3.39 (q, 1 H) 3.47-3.56 (m, 1 |
| | yl)pyridin-3-yl)picolinamide | H) 4.13-4.32 (m, 1 H) |
| | | 4.62 (dd, 1 H) 4.77 (s, 1 H) 5.43 (d, |
| | | 1 H) 7.30-7.39 (m, 3 H) |
| | | 7.70 (m, 1 H) 8.24 (t, 1 H) 8.40 (d, 1 |
| | | H) 8.44 (dd,1 H) 9.24 (s, 1 H) |
| | | 10.32 (s, 1 H) |
| 72 | 6-(2,6-difluorophenyl)-5-fluoro-N-(4- | |
| | ((2S,3S,5R,6R)-3-fluoro-5-hydroxy-5,6- | |
| | dimethyl-4-oxotetrahydro-2H-pyran-2- | |
| | yl)pyridin-3-yl)picolinamide | |
| 73 | 6-(2,6-difluorophenyl)-N-(4-((2S,4S,5S,6S)-5- | |
| | ethyl-4,5-dihydroxy-6-methyltetrahydro-2H- | |
| | pyran-2-yl)pyridin-3-yl)-5-fluoropicolinamide | |
| 74 | 6-(2,6-difluorophenyl)-N-(4-((2R,4R,5R,6R)- | |
| | 5-ethyl-4,5-dihydroxy-6-methyltetrahydro-2H- | |
| | pyran-2-yl)pyridin-3-yl)-5-fluoropicolinamide | |
| 75 | 6-(2,6-difluorophenyl)-N-(4-((2R,4R,5S,6R)- | |
| | 6-ethyl-4,5-dihydroxytetrahydro-2H-pyran-2- | |
| | yl)pyridin-3-yl)-5-fluoropicolinamide | |
| 76 | 6-(2,6-difluorophenyl)-N-(4-((2S,4S,5R,6S)-5- | |
| | ethyl-4,5-dihydroxy-6-methyltetrahydro-2H- | |
| | pyran-2-yl)pyridin-3-yl)-5-fluoropicolinamide | |
| 77 | 6-(2,6-difluorophenyl)-N-(4-((2R,4R,5S,6R)- | |
| | 5-ethyl-4,5-dihydroxy-6-methyltetrahydro-2H- | |
| | pyran-2-yl)pyridin-3-yl)-5-fluoropicolinamide | |
| 78 | 6-(2,6-difluorophenyl)-N-(4-((2S,4R,5S,6R)- | |
| | 4,5-dihydroxy-6-(hydroxymethyl)tetrahydro- | |
| | 2H-pyran-2-yl)pyridin-3-yl)-5- | |
| | fluoropicolinamide | |
| 79 | 6-(2,6-difluorophenyl)-N-(4-((2R,3S,4R)-3,4- | |
| | dihydroxy-2-(hydroxymethyl)-3,4-dihydro- | |
| | 2H-pyran-6-yl)pyridin-3-yl)-5- | |
| | fluoropicolinamide | |
| 80 | 2-(2,6-difluorophenyl)-N-(4-((2R,4R,5S,6R)- | |
| | 4,5-dihydroxy-6-(hydroxymethyl)tetrahydro- | |
| | 2H-pyran-2-yl)pyridin-3-yl)thiazole-4- | |
| | carboxamide | |
| 81 | 6-(2,6-difluorophenyl)-5-fluoro-N-(4- | 1H NMR (300 MHz, CDCl3) |
| | ((2S,3R,4R,5S,6R)-3,4,5-trihydroxy-5,6- | d: 10.59 (s, 1H), 9.10 (s, 1H), |
| | dimethyltetrahydro-2H-pyran-2-yl)pyridin-3- | 8.36 (dd, 1H), 8.22 (d, 1H), |
| | yl)picolinamide | 7.72 (dd, 1H), 7.43-7.50 (m, |
| | | 1H), 7.30 (d, H), 7.07 (dd, 1H), |
| | | 4.37 (d, 1H), 3.39-3.56 (m, 4H), |
| | | 1.03 (s, 3H), 0.97 (d, 3H), |
| | | 0.92 (d, 1H). |
| 82 | 6-(2,6-difluorophenyl)-5-fluoro-N-(4- | 1H NMR (300 MHz, CDCl3) |
| | ((2R,3S,4S,5R,6S)-3,4,5-trihydroxy-5,6- | d: 10.59 (s, 1H), 9.10 (s, 1H), |
| | dimethyltetrahydro-2H-pyran-2-yl)pyridin-3- | 8.36 (dd, 1H), 8.22 (d, 1H), |
| | yl)picolinamide | 7.72 (dd, 1H), 7.43-7.50 (m, |
| | | 1H), 7.30 (d, 1H), 7.07 (dd, |
| | | 1H), 4.37 (d, 1H), 3.39-3.56 (m, |
| | | 4H), 1.03 (s, 3H), 0.97 (d, 3H), |
| | | 0.92 (d, 1H). |
| 83 | 6-(2,6-difluorophenyl)-N-(4-((2R,4R,5S,6R)- | |
| | 4,5-dihydroxy-6-(hydroxymethyl)tetrahydro- | |
| | 2H-pyran-2-yl)pyridin-3-yl)-5- | |
| | fluoropicolinamide | |
| 84 | 3-amino-N-(4-((2R,4R,5S,6R)-4,5-dihydroxy- | |
| | 5,6-dimethyltetrahydro-2H-pyran-2- | |
| | yl)pyridin-3-yl)-6-phenylpyrazine-2- | |
| | carboxamide | |
| 85 | N-(4-((2R,4R,5S,6R)-4,5-dihydroxy-5,6- | |
| | dimethyltetrahydro-2H-pyran-2-yl)pyridin-3- | |
| | yl)-6-phenylpyrazine-2-carboxamide | |
| 86 | 3-amino-N-(4-((2S,4S,5R,6S)-4,5-dihydroxy- | |
| | 5,6-dimethyltetrahydro-2H-pyran-2- | |
| | yl)pyridin-3-yl)-6-phenylpyrazine-2- | |
| | carboxamide | |
| 87 | N-(4-((2S,4S,5R,6S)-4,5-dihydroxy-5,6- | |
| | dimethyltetrahydro-2H-pyran-2-yl)pyridin-3- | |
| | yl)-6-phenylpyrazine-2-carboxamide | |
| 88 | 2-(2,6-difluorophenyl)-N-(4-((2R,4S,5S)-4,5- | |
| | dihydroxy-6,6-dimethyltetrahydro-2H-pyran- | |
| | 2-yl)pyridin-3-yl)thiazole-4-carboxamide | |
| 89 | 6-(2,6-difluorophenyl)-N-(4-((2R,4S,5S)-4,5- | |
| | dihydroxy-6,6-dimethyltetrahydro-2H-pyran- | |
| | 2-yl)pyridin-3-yl)-5-fluoropicolinamide | |
| 90 | 2-(2,6-difluorophenyl)-N-(4-((2S,4R,5R)-4,5- | |
| | dihydroxy-6,6-dimethyltetrahydro-2H-pyran- | |
| | 2-yl)pyridin-3-yl)thiazole-4-carboxamide | |
| 91 | 6-(2,6-difluorophenyl)-N-(4-((2S,4R,5R)-4,5- | |
| | dihydroxy-6,6-dimethyltetrahydro-2H-pyran- | |
| | 2-yl)pyridin-3-yl)-5-fluoropicolinamide | |
| 92 | N-(4-((2R,4R,5S,6R)-4,5-dihydroxy-5,6- | |
| | dimethyltetrahydro-2H-pyran-2-yl)pyridin-3- | |
| | yl)-6-(2-fluorophenyl)picolinamide | |
| 93 | N-(4-((2R,4R,5S,6R)-4,5-dihydroxy-5,6- | |
| | dimethyltetrahydro-2H-pyran-2-yl)pyridin-3- | |
| | yl)-5-fluoro-6-(2-fluorophenyl)picolinamide | |
| 94 | 2-(2,6-difluorophenyl)-N-(4-((2S,4S,5R,6S)-4- | |
| | hydroxy-5,6-dimethyltetrahydro-2H-pyran-2- | |
| | yl)pyridin-3-yl)thiazole-4-carboxamide | |
| 95 | 2-(2,6-difluorophenyl)-N-(4-((2R,4R,5S,6R)- | |
| | 4-hydroxy-5,6-dimethyltetrahydro-2H-pyran- | |
| | 2-yl)pyridin-3-yl)thiazole-4-carboxamide | |
| 96 | 6-(2,6-difluorophenyl)-5-fluoro-N-(4- | |
| | ((2S,4S,5R,6S)-4-hydroxy-5,6- | |
| | dimethyltetrahydro-2H-pyran-2-yl)pyridin-3- | |
| | yl)picolinamide | |
| 97 | 6-(2,6-difluorophenyl)-5-fluoro-N-(4- | |
| | ((2R,4R,5S,6R)-4-hydroxy-5,6- | |
| | dimethyltetrahydro-2H-pyran-2-yl)pyridin-3- | |
| | yl)picolinamide | |
| 98 | 3-amino-6-(2,6-difluoro-3- | |
| | (isopropylcarbamoyl)phenyl)-N-(4- | |
| | ((2S,4R,5S,6R)-4,5-dihydroxy-5,6- | |
| | dimethyltetrahydro-2H-pyran-2-yl)pyridin-3- | |
| | yl)picolinamide | |
| 99 | 2-(2,6-difluorophenyl)-N-(4-((2R,4R)-4- | |
| | (hydroxymethyl)-1,3-dioxan-2-yl)pyridin-3- | |
| | yl)thiazole-4-carboxamide | |
| 100 | 6-(2,6-difluorophenyl)-5-fluoro-N-(4- | |
| | ((2R,4R)-4-(hydroxymethyl)-1,3-dioxan-2- | |
| | yl)pyridin-3-yl)picolinamide | |
| 101 | 6-(2,6-difluorophenyl)-5-fluoro-N-(4-((2S,4S)- | |
| | 4-(hydroxymethyl)-1,3-dioxolan-2-yl)pyridin- | |
| | 3-yl)picolinamide | |
| 102 | 2-(2,6-difluorophenyl)-N-(4-((2S,4S)-4- | |
| | (hydroxymethyl)-1,3-dioxan-2-yl)pyridin-3- | |
| | yl)thiazole-4-carboxamide | |
| 103 | 6-(2,6-difluorophenyl)-5-fluoro-N-(4-((2S,4S)- | |
| | 4-(hydroxymethyl)-1,3-dioxan-2-yl)pyridin-3- | |
| | yl)picolinamide | |
| 104 | 3-amino-N-(4-((2R,4R,5S,6R)-4,5-dihydroxy- | |
| | 5,6-dimethyltetrahydro-2H-pyran-2- | |
| | yl)pyridin-3-yl)-6-(2-fluoro-5- | |
| | (isopropylcarbamoyl)phenyl)picolinamide | |
| 105 | N-(4-((2R,4R,5S,6R)-4,5-dihydroxy-5,6- | |
| | dimethyltetrahydro-2H-pyran-2-yl)pyridin-3- | |
| | yl)-5-fluoro-6-(2-fluoro-5- | |
| | (isopropylcarbamoyl)phenyl)picolinamide | |
| 106 | N-(4-((2R,4R,5S,6R)-4,5-dihydroxy-5,6- | |
| | dimethyltetrahydro-2H-pyran-2-yl)pyridin-3- | |
| | yl)-6-(2-fluoro-5- | |
| | (isopropylcarbamoyl)phenyl)picolinamide | |
| 107 | 6-(2,6-difluorophenyl)-N-(4-((2S,4R,5R,6S)- | |
| | 4,5-dihydroxy-6-methyltetrahydro-2H-pyran- | |
| | 2-yl)pyridin-3-yl)-5-fluoropicolinamide | |
| 108 | 6-(2,6-difluorophenyl)-N-(4-((2R,4S,5S,6R)- | |
| | 4,5-dihydroxy-6-methyltetrahydro-2H-pyran- | |
| | 2-yl)pyridin-3-yl)-5-fluoropicolinamide | |
| 109 | 2-(2,6-difluorophenyl)-N-(4-((2R,4R,5S,6R)- | 1H NMR (400 MHz, DMSO- |
| | 4,5-dihydroxy-5,6-dimethyltetrahydro-2H- | d6) d: ppm 0.77 (s, 3 H) |
| | pyran-2-yl)pyridin-3-yl)thiazole-4- | 0.97 (d, 3 H) 1.49-1.60 (m, 2 H) |
| | carboxamide | 187-1.95 (m 2 H) |
| | | 3.29-3.36 (m, 2 H) 3.49-3.56 (m, 2 H) |
| | | 4.74-4.81 (m, 1 H) 7.34 (t, 1 |
| | | H) 7.49 (d, 1 H) 7.64-7.74 (m, |
| | | 1 H) 8.43 (d, 1 H) 8.82 (s, 1 H) |
| | | 9.34 (s, 1 H) 10.54 (s, 1 H) |
| 110 | 6-(2,6-difluorophenyl)-N-(4-((2R,4R,5S,6R)- | 1H NMR (400 MHz, DMSO- |
| | 4,5-dihydroxy-5,6-dimethyltetrahydro-2H- | d6) d: ppm 0.67 (d, 3 H) |
| | pyran-2-yl)pyridin-3-yl)-5-fluoropicolinamide | 0.69 (s, 3 H) 1.48-1.62 (m, 2 H) |
| | | 1.84-1.93 (m, 2 H) |
| | | 3.19-3.28 (m, 2 H) 3.47-3.54 (m, 2 H) |
| | | 4.68-4.76 (m, 1 H) 7.34 (t, 1 |
| | | H) 7.48 (d, 1 H) 7.65-7.75 (m, |
| | | 1 H) 8.21-8.28 (m, 1 H) |
| | | 8.44 (q, 1 H) 9.29 (s, 1 H) 10.60 (s, 1 |
| | | H) |
| 111 | 2-(2,6-difluorophenyl)-N-(4-((2S,4S,5R,6S)- | |
| | 4,5-dihydroxy-5,6-dimethyltetrahydro-2H- | |
| | pyran-2-yl)pyridin-3-yl)thiazole-4- | |
| | carboxamide | |
| 112 | 6-(2,6-difluorophenyl)-N-(4-((2S,4S,5R,6S)- | |
| | 4,5-dihydroxy-5,6-dimethyltetrahydro-2H- | |
| | pyran-2-yl)pyridin-3-yl)-5-fluoropicolinamide | |
| 113 | N-(4-((2S,4S,5R,6S)-4-amino-5-hydroxy-6- | 1H NMR (400 MHz, DMSO- |
| | methyltetrahydro-2H-pyran-2-yl)pyridin-3-yl)- | d6) d: ppm 1.06 (d, 3 H) |
| | 2-(2,6-difluorophenyl)thiazole-4-carboxamide | 2.06-2.17 (m, 1 H) 2.17-2.34 (m, 2 |
| | | H) 3.30-3.43 (m, 1 H) 3.58 (br. |
| | | s., 1 H) 3.66-3.78 (m, 2 H) |
| | | 4.94 (d, 1 H) 7.26-7.39 (m, 2 |
| | | H) 7.69 (s, 1 H) 7.93 (br. s., 2 |
| | | H) 8.42 (d, 1 H) 8.82 (s, 1 H) |
| | | 9.23 (s, 1 H) 10.34 (s, 1 H) |
| 114 | N-(4-((2R,4R,5S,6R)-4-amino-5-hydroxy-6- | 1H NMR (400 MHz, DMSO- |
| | methyltetrahydro-2H-pyran-2-yl)pyridin-3-yl)- | d6) d ppm 1.07 (d, 3 H) |
| | 2-(2,6-difluorophenyl)thiazole-4-carboxamide | 2.07-2.17 (m, 1 H) 2.18-2.30 (m, 2 |
| | | H) 3.38 (dd, 1 H) 3.58 (br. s., 1 |
| | | H) 3.66-3.78 (m, 2 H) |
| | | 4.91-5.00 (m, 1 H) 7.29-7.39 (m, 2 |
| | | H) 7.64-7.76 (m, 1 H) 7.94 (br. |
| | | s., 2 H) 8.43 (d, 1 H) 8.82 (s, 1 |
| | | H) 9.25 (s, 1 H) 10.36 (s, 1 H) |
| 115 | N-(4-((2S,4S,5R,6S)-4-amino-5-hydroxy-6- | 1H NMR (400 MHz, DMSO- |
| | methyltetrahydro-2H-pyran-2-yl)pyridin-3-yl)- | d6) d ppm 0.74 (d, 3 H) |
| | 6-(2,6-difluorophenyl)-5-fluoropicolinamide | 2.03-2.13 (m, 1 H) 2.16-2.29 (m, 2 |
| | | H) 3.18-3.30 (m, 1 H) |
| | | 3.49-3.66 (m, 3 H) 4.85-4.93 (m, 1 |
| | | H) 7.27-7.38 (m, 2 H) |
| | | 7.62-7.75 (m, 1 H) 7.90 (br. s., 2 H) |
| | | 8.24 (t, 1 H) 8.39-8.48 (m, 2 |
| | | H) 9.16 (s, 1 H) 10.41 (s, 1 H) |
| 116 | N-(4-((2R,4R,5S,6R)-4-amino-5-hydroxy-6- | 1H NMR (400 MHz, DMSO- |
| | methyltetrahydro-2H-pyran-2-yl)pyridin-3-yl)- | d6) d ppm 0.75 (d, 3 H) |
| | 6-(2,6-difluorophenyl)-5-fluoropicolinamide | 2.07-2.15 (m, 1 H) 2.19-2.29 (m, 2 |
| | | H) 3.24-3.30 (m, 1 H) |
| | | 3.52-3.64 (m, 3 H) 4.88-4.94 (m, 1 |
| | | H) 7.31-7.40 (m, 2 H) 7.71 (s, |
| | | 1 H) 7.92 (br. s., 2 H) 8.26 (t, 1 |
| | | H) 8.42-8.49 (m, 2 H) 9.18 (s, |
| | | 1 H) 10.43 (s, 1 H) |
| 117 | N-(4-(1,3-dioxan-2-yl)pyridin-3-yl)-2-(2,6- | |
| | difluorophenyl)thiazole-4-carboxamide | |
| 118 | N-(4-(1,3-dioxan-2-yl)pyridin-3-yl)-6-(2,6- | |
| | difluorophenyl)-5-fluoropicolinamide | |
| 119 | 6-(2,6-difluorophenyl)-N-(4-((2R,4R,5S,6R)- | |
| | 4,5-dihydroxy-6-methyltetrahydro-2H-pyran- | |
| | 2-yl)pyridin-3-yl)-5-fluoropicolinamide | |
| 120 | 6-(2,6-difluorophenyl)-N-(4-((2S,4S,5R,6S)- | |
| | 4,5-dihydroxy-6-methyltetrahydro-2H-pyran- | |
| | 2-yl)pyridin-3-yl)-5-fluoropicolinamide | |
| 121 | N-(4-((2R,4S,6R)-4-amino-6- | |
| | methyltetrahydro-2H-pyran-2-yl)pyridin-3-yl)- | |
| | 2-(2,6-difluorophenyl)thiazole-4-carboxamide | |
| 122 | N-(4-((2R,4S,6R)-4-amino-6- | |
| | methyltetrahydro-2H-pyran-2-yl)pyridin-3-yl)- | |
| | 6-(2,6-difluorophenyl)-5-fluoropicolinamide | |
| 123 | N-(4-((2S,4R,6S)-4-amino-6- | |
| | methyltetrahydro-2H-pyran-2-yl)pyridin-3-yl)- | |
| | 2-(2,6-difluorophenyl)thiazole-4-carboxamide | |
| 124 | N-(4-((2S,4R,6S)-4-amino-6- | |
| | methyltetrahydro-2H-pyran-2-yl)pyridin-3-yl)- | |
| | 6-(2,6-difluorophenyl)-5-fluoropicolinamide | |
| 125 | 2-(2,6-difluorophenyl)-N-(4-((2R,4S,6R)-4- | |
| | hydroxy-6-methyltetrahydro-2H-pyran-2- | |
| | yl)pyridin-3-yl)thiazole-4-carboxamide | |
| 126 | 6-(2,6-difluorophenyl)-5-fluoro-N-(4- | |
| | ((2R,4S,6R)-4-hydroxy-6-methyltetrahydro- | |
| | 2H-pyran-2-yl)pyridin-3-yl)picolinamide | |
| 127 | 2-(2,6-difluorophenyl)-N-(4-((2S,4R,6S)-4- | |
| | hydroxy-6-methyltetrahydro-2H-pyran-2- | |
| | yl)pyridin-3-yl)thiazole-4-carboxamide | |
| 128 | 6-(2,6-difluorophenyl)-5-fluoro-N-(4- | |
| | ((2S,4R,6S)-4-hydroxy-6-methyltetrahydro- | |
| | 2H-pyran-2-yl)pyridin-3-yl)picolinamide | |
| 129 | 6-(2,6-difluorophenyl)-5-fluoro-N-(4- | |
| | ((2R,4S)-4-hydroxytetrahydro-2H-pyran-2- | |
| | yl)pyridin-3-yl)picolinamide | |
| 130 | 2-(2,6-difluorophenyl)-N-(4-((2S,4R)-4- | |
| | hydroxytetrahydro-2H-pyran-2-yl)pyridin-3- | |
| | yl)thiazole-4-carboxamide | |
| 131 | 2-(2,6-difluorophenyl)-N-(4-((2R,4S)-4- | |
| | hydroxytetrahydro-2H-pyran-2-yl)pyridin-3- | |
| | yl)thiazole-4-carboxamide | |
| 132 | 6-(2,6-difluorophenyl)-5-fluoro-N-(4- | |
| | ((2S,4R)-4-hydroxytetrahydro-2H-pyran-2- | |
| | yl)pyridin-3-yl)picolinamide | |
| 133 | (S)-6-(2,6-difluorophenyl)-5-fluoro-N-(4- | |
| | (tetrahydro-2H-pyran-2-yl)pyridin-3- | |
| | yl)picolinamide | |
| 134 | (R)-6-(2,6-difluorophenyl)-5-fluoro-N-(4- | |
| | (tetrahydro-2H-pyran-2-yl)pyridin-3- | |
| | yl)picolinamide | |
| 135 | 2-(2,6-difluorophenyl)-N-(4-((2S,4S,5R,6S)- | |
| | 4,5-dihydroxy-6-methyltetrahydro-2H-pyran- | |
| | 2-yl)pyridin-3-yl)thiazole-4-carboxamide | |
| 136 | 2-(2,6-difluorophenyl)-N-(4-((2R,4R,5S,6R)- | |
| | 4,5-dihydroxy-6-methyltetrahydro-2H-pyran- | |
| | 2-yl)pyridin-3-yl)thiazole-4-carboxamide | |
| 137 | N-(4-((2R,4R,5S,6R)-6-(cyanomethyl)-4,5- | |
| | dihydroxytetrahydro-2H-pyran-2-yl)pyridin-3- | |
| | yl)-6-(2,6-difluorophenyl)-5- | |
| | fluoropicolinamide | |
| 138 | 5-amino-N-(4-((2R,4R,5S,6R)-6- | |
| | (cyanomethyl)-4,5-dihydroxytetrahydro-2H- | |
| | pyran-2-yl)pyridin-3-yl)-2-(2,6- | |
| | difluorophenyl)pyrimidine-4-carboxamide | |
| 139 | 3-amino-N-(4-((2R,4R,5S,6R)-6- | |
| | (cyanomethyl)-4,5-dihydroxytetrahydro-2H- | |
| | pyran-2-yl)pyridin-3-yl)-6-(2,6- | |
| | difluorophenyl)-5-fluoropicolinamide | |
| 140 | 3-amino-N-(4-((2R,4R,5S,6R)-6-ethyl-4,5- | |
| | dihydroxytetrahydro-2H-pyran-2-yl)pyridin-3- | |
| | yl)-6-(thiazol-2-yl)picolinamide | |
| 141 | N-(4-((2R,4R,5S,6S)-6-carbamoyl-4,5- | |
| | dihydroxytetrahydro-2H-pyran-2-yl)pyridin-3- | |
| | yl)-6-(2,6-difluorophenyl)-5- | |
| | fluoropicolinamide | |
| 142 | N-(4-((2R,4R,5S,6R)-6-(2-amino-2-oxoethyl)- | |
| | 4,5-dihydroxytetrahydro-2H-pyran-2- | |
| | yl)pyridin-3-yl)-6-(2,6-difluorophenyl)-5- | |
| | fluoropicolinamide | |
| 143 | 5-amino-2-(2,6-difluorophenyl)-N-(4- | 400 (DMSOd6) d 10.60 (s, 1H), |
| | ((2R,4R,5S,6R)-5-ethyl-4,5-dihydroxy-6- | 9.24 (s, 1H), 8.70 (s, 1H), |
| | methyltetrahydro-2H-pyran-2-yl)pyridin-3- | 8.50 (d, J = 5.2, 1H), 7.62 (d, J = 5.3, |
| | yl)pyrimidine-4-carboxamide | 1H), 7.49-7.55 (m, 1H), 7.19 (t, |
| | | J = 6.0, 2H), 4.76 (dd, J = 11.2, |
| | | 1.2, 1H), 3.54-3.58 (m, 1H), |
| | | 3.22 (q, J = 6.4, 1H), |
| | | 1.87-1.92 (m, 1H), 1.63 (dd, J = 12.4, 12.4, |
| | | 1H), 1.41-1.49 (m, 1H), |
| | | 1.21-1.28 (m, 1H), 0.71 (t, J = 8.0, |
| | | 3H), 0.69 (d, J = 6.4, 3H). |
| 144 | 5-amino-2-(2,6-difluorophenyl)-N-(4- | |
| | ((2R,3S,4R)-3,4-dihydroxy-2-vinyl-3,4- | |
| | dihydro-2H-pyran-6-yl)pyridin-3- | |
| | yl)pyrimidine-4-carboxamide | |
| 145 | 3-amino-N-(4-((2R,4R,5S,6R)-6-ethyl-4,5- | |
| | dihydroxytetrahydro-2H-pyran-2-yl)pyridin-3- | |
| | yl)-6-(thiazol-2-yl)picolinamide | |
| 146 | 5-amino-2-(2,6-difluorophenyl)-N-(4- | |
| | ((2R,4R,5S,6R)-4,5-dihydroxy-5,6- | |
| | dimethyltetrahydro-2H-pyran-2-yl)pyridin-3- | |
| | yl)pyrimidine-4-carboxamide | |
| 147 | 3-amino-N-(4-((2R,4R,5S,6R)-4,5-dihydroxy- | |
| | 5,6-dimethyltetrahydro-2H-pyran-2- | |
| | yl)pyridin-3-yl)-6-(thiazol-2-yl)picolinamide | |
| 148 | 5-amino-N-(4-((2R,4R,5S,6R)-6-cyano-4,5- | |
| | dihydroxytetrahydro-2H-pyran-2-yl)pyridin-3- | |
| | yl)-2-(2,6-difluorophenyl)pyrimidine-4- | |
| | carboxamide | |
| 149 | N-(4-((2R,4R,5S,6R)-6-cyano-4,5- | |
| | dihydroxytetrahydro-2H-pyran-2-yl)pyridin-3- | |
| | yl)-2-(2,6-difluorophenyl)pyrimidine-4- | |
| | carboxamide | |
| 150 | 3-amino-N-(4-((2R,4R,5S,6R)-6-cyano-4,5- | |
| | dihydroxytetrahydro-2H-pyran-2-yl)pyridin-3- | |
| | yl)-6-(thiazol-2-yl)picolinamide | |
| 151 | 3-amino-N-(4-((2S,3R)-2-(chloromethyl)-3- | |
| | hydroxy-4-oxo-3,4-dihydro-2H-pyran-6- | |
| | yl)pyridin-3-yl)-5-fluoro-6-(2- | |
| | fluorophenyl)picolinamide | |
| 152 | 3-amino-N-(4-((2R,4R,5S,6R)-6- | |
| | (cyanomethyl)-4,5-dihydroxytetrahydro-2H- | |
| | pyran-2-yl)pyridin-3-yl)-6-(2,6- | |
| | difluorophenyl)-5-fluoropicolinamide | |
| 153 | 5-amino-N-(4-((2R,4R,5S,6R)-6- | |
| | (cyanomethyl)-4,5-dihydroxytetrahydro-2H- | |
| | pyran-2-yl)pyridin-3-yl)-2-(2,6- | |
| | difluorophenyl)pyrimidine-4-carboxamide | |
| 155 | 5-amino-N-(4-((2R,4R,5S,6R)-5-ethyl-4,5- | |
| | dihydroxy-6-methyltetrahydro-2H-pyran-2- | |
| | yl)pyridin-3-yl)-3′-fluoro-2,2′-bipyridine-6- | |
| | carboxamide | |
| 156 | 3-amino-6-(1,5-dimethyl-1H-pyrazol-4-yl)-N- | |
| | (4-((2R,4R,5S,6R)-5-ethyl-4,5-dihydroxy-6- | |
| | methyltetrahydro-2H-pyran-2-yl)pyridin-3- | |
| | yl)picolinamide | |
| 157 | 5-amino-N-(4-((2R,4R,5S,6R)-5-ethyl-4,5- | |
| | dihydroxy-6-methyltetrahydro-2H-pyran-2- | |
| | yl)pyridin-3-yl)-3′-fluoro-2,4′-bipyridine-6- | |
| | carboxamide | |
| 158 | 3-amino-N-(4-((2R,4R,5S,6R)-5-ethyl-4,5- | |
| | dihydroxy-6-methyltetrahydro-2H-pyran-2- | |
| | yl)pyridin-3-yl)picolinamide | |
| 159 | N-(4-((2R,4R,5S,6R)-6-(2-amino-2-oxoethyl)- | |
| | 4,5-dihydroxytetrahydro-2H-pyran-2- | |
| | yl)pyridin-3-yl)-6-(2,6-difluorophenyl)-5- | |
| | fluoropicolinamide | |
| 160 | 3-amino-N-(4-((2R,4R,5S,6R)-5-ethyl-4,5- | |
| | dihydroxy-6-methyltetrahydro-2H-pyran-2- | |
| | yl)pyridin-3-yl)-6-(pyridazin-4- | |
| | yl)picolinamide | |
| 161 | 5-amino-2-(2,6-difluorophenyl)-N-(4- | |
| | ((2R,4R,5S,6R)-4,5-dihydroxy-6- | |
| | vinyltetrahydro-2H-pyran-2-yl)pyridin-3- | |
| | yl)pyrimidine-4-carboxamide | |
| 162 | 6-(2,6-difluorophenyl)-N-(4-((2R,4R,5S,6R)- | |
| | 6-ethyl-4,5-dihydroxytetrahydro-2H-pyran-2- | |
| | yl)pyrimidin-5-yl)-5-fluoropicolinamide | |
| 163 | 6-(2,6-difluorophenyl)-N-(4-((2S,4S,5R,6S)-6- | |
| | ethyl-4,5-dihydroxy-5-methyltetrahydro-2H- | |
| | pyran-2-yl)pyridin-3-yl)-5-fluoropicolinamide | |
| 164 | 6-(2,6-difluorophenyl)-N-(4-((2R,4R,5S,6R)- | |
| | 6-ethyl-4,5-dihydroxy-5-methyltetrahydro-2H- | |
| | pyran-2-yl)pyridin-3-yl)-5-fluoropicolinamide | |
| 165 | 3-amino-6-(2,6-difluorophenyl)-N-(4- | |
| | ((2S,4S,5R,6S)-6-ethyl-4,5-dihydroxy-5- | |
| | methyltetrahydro-2H-pyran-2-yl)pyridin-3-yl)- | |
| | 5-fluoropicolinamide | |
| 166 | 3-amino-6-(2,6-difluorophenyl)-N-(4- | |
| | ((2R,4R,5S,6R)-6-ethyl-4,5-dihydroxy-5- | |
| | methyltetrahydro-2H-pyran-2-yl)pyridin-3-yl)- | |
| | 5-fluoropicolinamide | |
| 167 | 5-amino-2-(2,6-difluorophenyl)-N-(4- | |
| | ((2S,4S,5R,6S)-6-ethyl-4,5-dihydroxy-5- | |
| | methyltetrahydro-2H-pyran-2-yl)pyridin-3- | |
| | yl)pyrimidine-4-carboxamide | |
| 168 | 5-amino-2-(2,6-difluorophenyl)-N-(4- | |
| | ((2R,4R,5S,6R)-6-ethyl-4,5-dihydroxy-5- | |
| | methyltetrahydro-2H-pyran-2-yl)pyridin-3- | |
| | yl)pyrimidine-4-carboxamide | |
| 169 | 5-amino-2-(2,6-difluorophenyl)-N-(4- | |
| | ((2R,4R,5S,6R)-4,5-dihydroxy-6- | |
| | propyltetrahydro-2H-pyran-2-yl)pyridin-3- | |
| | yl)pyrimidine-4-carboxamide | |
| 170 | N-(4-((2R,4R,5S,6R)-6-ethyl-4,5- | |
| | dihydroxytetrahydro-2H-pyran-2-yl)pyridin-3- | |
| | yl)-4-oxo-5-phenyl-1,4-dihydropyridine-3- | |
| | carboxamide | |
| 171 | N-(4-((2S,4S,4aR,8aS)-4,4a- | |
| | dihydroxyoctahydro-2H-chromen-2- | |
| | yl)pyridin-3-yl)-4-oxo-5-phenyl-1,4- | |
| | dihydropyridine-3-carboxamide | |
| 172 | 5-amino-2-(2,6-difluorophenyl)-N-(4- | |
| | ((2R,4R,4aS,8aR)-4,4a-dihydroxyoctahydro- | |
| | 2H-chromen-2-yl)pyridin-3-yl)pyrimidine-4- | |
| | carboxamide | |
| 173 | 3-amino-N-(4-((2R,4R,4aS,8aR)-4,4a- | |
| | dihydroxyoctahydro-2H-chromen-2- | |
| | yl)pyridin-3-yl)-6-phenylpyrazine-2- | |
| | carboxamide | |
| 174 | 6-(2,6-difluorophenyl)-N-(4- | |
| | ((2R,4R,4aS,8aR)-4,4a-dihydroxyoctahydro- | |
| | 2H-chromen-2-yl)pyridin-3-yl)-5- | |
| | fluoropicolinamide | |
| 175 | 5-amino-2-(2,6-difluorophenyl)-N-(4- | |
| | ((2S,4S,4aR,8aS)-4,4a-dihydroxyoctahydro- | |
| | 2H-chromen-2-yl)pyridin-3-yl)pyrimidine-4- | |
| | carboxamide | |
| 176 | 3-amino-N-(4-((2S,4S,4aR,8aS)-4,4a- | |
| | dihydroxyoctahydro-2H-chromen-2- | |
| | yl)pyridin-3-yl)-6-phenylpyrazine-2- | |
| | carboxamide | |
| 177 | 6-(2,6-difluorophenyl)-N-(4-((2S,4S,4aR,8aS)- | |
| | 4,4a-dihydroxyoctahydro-2H-chromen-2- | |
| | yl)pyridin-3-yl)-5-fluoropicolinamide | |
| 178 | N-(4-((2R,4R,4aS,8aR)-4,4a- | |
| | dihydroxyoctahydro-2H-chromen-2- | |
| | yl)pyridin-3-yl)-4-oxo-5-phenyl-1,4- | |
| | dihydropyridine-3-carboxamide | |
| 179 | N-(4-((2R,4R,5S,6R)-5-ethyl-4,5-dihydroxy-6- | |
| | methyltetrahydro-2H-pyran-2-yl)pyridin-3-yl)- | |
| | 4-oxo-5-phenyl-1,4-dihydropyridine-3- | |
| | carboxamide | |
| 180 | 5-amino-2-(2,6-difluorophenyl)-N-(4- | |
| | ((2R,4R,4aS,7aR)-4,4a- | |
| | dihydroxyoctahydrocyclopenta[b]pyran-2- | |
| | yl)pyridin-3-yl)pyrimidine-4-carboxamide | |
| 181 | N-(4-((2R,4R,4aS,7aR)-4,4a- | |
| | dihydroxyoctahydrocyclopenta[b]pyran-2- | |
| | yl)pyridin-3-yl)-4-oxo-5-phenyl-1,4- | |
| | dihydropyridine-3-carboxamide | |
| 182 | 5-amino-2-(2,6-difluorophenyl)-N-(4- | |
| | ((2S,4S,4aR,7aS)-4,4a- | |
| | dihydroxyoctahydrocyclopenta[b]pyran-2- | |
| | yl)pyridin-3-yl)pyrimidine-4-carboxamide | |
| 183 | 3-amino-N-(4-((2R,4R,4aS,7aR)-4-amino-4a- | |
| | hydroxyoctahydrocyclopenta[b] pyran-2- | |
| | yl)pyridin-3-yl)-6-phenylpyrazine-2- | |
| | carboxamide | |
| 184 | N-(4-((2S,4S,4aR,7aS)-4,4a- | |
| | dihydroxyoctahydrocyclopenta[b]pyran-2- | |
| | yl)pyridin-3-yl)-4-oxo-5-phenyl-1,4- | |
| | dihydropyridine-3-carboxamide | |
| 185 | 3-amino-N-(4-((2S,4S,4aR,7aS)-4- | |
| | amino-4a- | |
| | hydroxyoctahydrocyclopenta[b]pyran-2- | |
| | yl)pyridin-3-yl)-6-phenylpyrazine-2- | |
| | carboxamide | |
| 186 | 3-amino-N-(4-((2S,4S,4aR,7aS)-4,4a- | |
| | dihydroxyoctahydrocyclopenta[b]pyran-2- | |
| | yl)pyridin-3-yl)-6-phenylpyrazine-2- | |
| | carboxamide | |
| 187 | 3-amino-N-(4-((2R,4R,5S,6R)-4-amino-5- | 1H NMR (400 MHz, <cd3od>) |
| | ethyl-5-hydroxy-6-methyltetrahydro-2H- | d ppm 0.74 (t, J = 7.63 Hz, 3 H) |
| | pyran-2-yl)pyridin-3-yl)-6-phenylpyrazine-2-H) | 1.06 (d, J = 6.65 Hz, 3 H) |
| | carboxamide | 1.33-1.46 (m, 1 H) 1.61 (dq, J = 15.16, |
| | | 7.73 Hz, 1 H) 1.84-2.01 (m, 1 |
| | | H) 2.12 (dt, J = 12.91, 3.33 Hz, 1 |
| | | 3.52 (q, J = 6.26 Hz, 1 H) |
| | | 4.93 (m, J = 9.40 Hz, 2 H) |
| | | 7.41 (d, J = 7.43 Hz, 1 H) |
| | | 7.43-7.51 (m, 2 H) 7.56 (d, J = 5.48 Hz, 1 |
| | | H) 7.97 (d, J = 7.43 Hz, 2 H) |
| | | 8.45 (d, J = 5.48 Hz, 1 H) |
| | | 8.78 (s, 1 H) 9.07 (s, 1 H) |
| 188 | 6-(2,6-difluorophenyl)-N-(4-((2S,4S,4aR,7aS)- | |
| | 4,4a-dihydroxyoctahydrocyclopenta[b]pyran- | |
| | 2-yl)pyridin-3-yl)-5-fluoropicolinamide | |
| 189 | 3-amino-N-(4-((2S,4S,5R,6S)-4-amino-5- | 1H NMR (400 MHz, <cd3od>) |
| | ethyl-5-hydroxy-6-methyltetrahydro-2H- | d ppm 0.73 (t, J = 7.83 Hz, 3 H) |
| | pyran-2-yl)pyridin-3-yl)-6-phenylpyrazine-2- | 1.06 (d, J = 6.26 Hz, 3 H) |
| | carboxamide | 1.39 (dq, J = 15.21, 7.58 Hz, 1 H) |
| | | 1.52-1.69 (m, 1 H) |
| | | 1.86-2.01 (m, 1 H) 2.07-2.18 (m, 1 H) |
| | | 3.52 (q, J = 6.52 Hz, 1 H) |
| | | 4.92 (dd, J = 11.35, 2.35 Hz, 2 H) |
| | | 7.37-7.43 (m, 1 H) |
| | | 7.45-7.51 (m, 2 H) 7.54 (d, J = 5.09 Hz, 1 |
| | | H) 7.97 (d, J = 7.04 Hz, 2 H) |
| | | 8.44 (d, J = 5.09 Hz, 1 H) |
| | | 8.78 (s, 1 H) 9.06 (s, 1 H) |
| 190 | N-(4-((2R,4R,4aS,8aR)-4,4a- | |
| | dihydroxyoctahydro-2H-chromen-2- | |
| | yl)pyridin-3-yl)-2-(2-fluorophenyl)-3-oxo-2,3- | |
| | dihydropyridazine-4-carboxamide | |
| 191 | N-(4-((2S,4S,4aR,8aS)-4,4a- | |
| | dihydroxyoctahydro-2H-chromen-2- | |
| | yl)pyridin-3-yl)-2-(2-fluorophenyl)-3-oxo-2,3- | |
| | dihydropyridazine-4-carboxamide | |
| 192 | N-(4-((2S,4S,4aR,7aS)-4,4a- | |
| | dihydroxyoctahydrocyclopenta[b]pyran-2- | |
| | yl)pyridin-3-yl)-2-oxo-1-phenyl-1,2- | |
| | dihydropyridine-3-carboxamide | |
| 193 | N-(4-((2R,4R,5S,6R)-6-ethyl-4,5- | |
| | dihydroxytetrahydro-2H-pyran-2-yl)pyridin-3- | |
| | yl)-2-oxo-1-phenyl-1,2-dihydropyridine-3- | |
| | carboxamide | |
| 194 | N-(4-((2R,4R,4aS,7aR)-4,4a- | |
| | dihydroxyoctahydrocyclopenta[b]pyran-2- | |
| | yl)pyridin-3-yl)-2-oxo-1-phenyl-1,2- | |
| | dihydropyridine-3-carboxamide | |
| 195 | 3-amino-N-(4-((2R,4R,4aS,7aR)-4,4a- | |
| | dihydroxyoctahydrocyclopenta[b]pyran-2- | |
| | yl)pyridin-3-yl)-6-phenylpyrazine-2- | |
| | carboxamide | |
| 196 | 6-(2,6-difluorophenyl)-5-fluoro-N-(4- | |
| | ((2R,4R,4aS,8aR)-4-hydroxy-6-oxooctahydro- | |
| | 2H-pyrano[3,2-b]pyridin-2-yl)pyridin-3- | |
| | yl)picolinamide | |
| 197 | 3-amino-N-(4-((2R,4R,4aS,8aR)-4-amino-4a- | 1H NMR (400 MHz, <cd3od>) |
| | hydroxyoctahydro-2H-chromen-2-yl)pyridin- | d ppm 1.18-1.28 (m, 1 H) |
| | 3-yl)-6-phenylpyrazine-2-carboxamide | 1.34 (d, J = 13.30 Hz, 1 H) |
| | | 1.40-1.83 (m, 7 H) 1.88-2.04 (m, 1 H) |
| | | 2.22-2.35 (m, 1 H) 3.53 (s, 1 |
| | | H) 4.99 (dd, J = 11.74, 2.35 Hz, |
| | | 1 H) 7.36-7.43 (m, 1 H) |
| | | 7.45-7.53 (m, 2 H) 7.68 (d, J = 5.09 Hz, |
| | | 1 H) 7.93-8.03 (m, 2 H) |
| | | 8.50 (d, J = 5.09 Hz, 1 H) |
| | | 9.02 (s, 1 H) |
| 198 | 6-(2,6-difluorophenyl)-5-fluoro-N-(4- | |
| | ((2R,4R,4aR,8aR)-4-hydroxy-6-oxooctahydro- | |
| | 2H-pyrano[3,2-b]pyridin-2-yl)pyridin-3- | |
| | yl)picolinamide | |
| 199 | 3-amino-N-(4-((2R,4R,4aR,7aR)-4- | 1H NMR (400 MHz, <cd3od>) |
| | aminooctahydrocyclopenta[b]pyran-2- | d ppm 1.39-1.75 (m, 7H) |
| | yl)pyridin-3-yl)-6-phenylpyrazine-2- | 1.86 (q, J = 12.39 Hz, 1 H) 2.09 (dd, |
| | carboxamide | J = 10.56, 4.70 Hz, 2 H) 3.84 (dt, |
| | | J = 12.13, 4.89 Hz, 1 H) 4.19 (t, |
| | | J = 3.52 Hz, 1 H) 7.36-7.43 (m, |
| | | 1 H) 7.45-7.52 (m, 2 H) |
| | | 7.64 (d, J = 5.48 Hz, 1 H) 7.97 (m, |
| | | J = 7.04 Hz, 2 H) 8.49 (d, J = 5.48 Hz, |
| | | 1 H) 8.76 (s, 1 H) 9.18 (s, 1 |
| | | H) |
| 200 | N-(4-((2R,4R,5R,6R)-5-cyano-6-ethyl-4- | |
| | hydroxytetrahydro-2H-pyran-2-yl)pyridin-3- | |
| | yl)-6-(2,6-difluorophenyl)-5- | |
| | fluoropicolinamide | |
| 201 | N-(4-((2R,4R,4aS,7aR)-4,4a- | |
| | dihydroxyoctahydrocyclopenta[b]pyran-2- | |
| | yl)pyridin-3-yl)-2-(2-fluorophenyl)-3-oxo-2,3- | |
| | dihydropyridazine-4-carboxamide | |
| 202 | N-(4-((2R,4R,5S,6R)-6-ethyl-4,5- | |
| | dihydroxytetrahydro-2H-pyran-2-yl)pyridin-3- | |
| | yl)-5-(2-fluorophenyl)-4-oxo-1,4- | |
| | dihydropyridine-3-carboxamide | |
| 203 | N-(4-((2S,4S,4aR,7aS)-4,4a- | |
| | dihydroxyoctahydrocyclopenta[b]pyran-2- | |
| | yl)pyridin-3-yl)-2-(2-fluorophenyl)-3-oxo-2,3- | |
| | dihydropyridazine-4-carboxamide | |
| 204 | 6-(2,6-difluorophenyl)-N-(4- | |
| | ((2R,4R,4aS,7aR)-4,4a- | |
| | dihydroxyoctahydrocyclopenta[b]pyran-2- | |
| | yl)pyridin-3-yl)-5-fluoropicolinamide | |
| 205 | 5-amino-N-(4-((2S,4S,5R,6S)-6-cyclopropyl- | |
| | 4,5-dihydroxytetrahydro-2H-pyran-2- | |
| | yl)pyridin-3-yl)-2-(2,6- | |
| | difluorophenyl)pyrimidine-4-carboxamide | |
| 206 | N-(4-((2S,4S,5R,6S)-6-cyclopropyl-4,5- | |
| | dihydroxytetrahydro-2H-pyran-2-yl)pyridin-3- | |
| | yl)-6-(2,6-difluorophenyl)picolinamide | |
| 207 | 5-amino-N-(4-((2R,4R,5S,6R)-6-cyclopropyl- | |
| | 4,5-dihydroxytetrahydro-2H-pyran-2- | |
| | yl)pyridin-3-yl)-2-(2,6- | |
| | difluorophenyl)pyrimidine-4-carboxamide | |
| 208 | N-(4-((2R,4R,5S,6R)-6-cyclopropyl-4,5- | |
| | dihydroxytetrahydro-2H-pyran-2-yl)pyridin-3- | |
| | yl)-6-(2,6-difluorophenyl)picolinamide | |
| 209 | N-(4-((2S,4S,5R,6S)-6-cyclopropyl-4,5- | |
| | dihydroxytetrahydro-2H-pyran-2-yl)pyridin-3- | |
| | yl)-6-(2,6-difluorophenyl)-5- | |
| | fluoropicolinamide | |
| 210 | N-(4-((2R,4R,4aS,7aR)-4-amino-4a- | |
| | hydroxyoctahydrocyclopenta[b]pyran-2- | |
| | yl)pyridin-3-yl)-6-(2,6-difluorophenyl)-5- | |
| | fluoropicolinamide | |
| 211 | N-(4-((2R,4R,5S,6R)-6-cyclopropyl-4,5- | |
| | dihydroxytetrahydro-2H-pyran-2-yl)pyridin-3- | |
| | yl)-6-(2,6-difluorophenyl)-5- | |
| | fluoropicolinamide | |
| 212 | N-(4-((2S,4S,4aR,7aS)-4-amino-4a- | |
| | hydroxyoctahydrocyclopenta[b]pyran-2- | |
| | yl)pyridin-3-yl)-6-(2,6-difluorophenyl)-5- | |
| | fluoropicolinamide | |
| 213 | 5-amino-N-(4-((2R,4R,4aS,7aR)-4-amino-4a- | |
| | hydroxyoctahydrocyclopenta[b]pyran-2- | |
| | yl)pyridin-3-yl)-2-(2,6- | |
| | difluorophenyl)pyrimidine-4-carboxamide | |
| 214 | 5-amino-N-(4-((2S,4S,4aR,7aS)-4-amino-4a- | |
| | hydroxyoctahydrocyclopenta[b]pyran-2- | |
| | yl)pyridin-3-yl)-2-(2,6- | |
| | difluorophenyl)pyrimidine-4-carboxamide | |
| 215 | 3-amino-N-(4-((2R,4R,4aS,7aR)-4-amino-4a- | |
| | hydroxyoctahydrocyclopenta[b]pyran-2- | |
| | yl)pyridin-3-yl)-6-(2,6- | |
| | difluorophenyl)picolinamide | |
| 216 | 3-amino-N-(4-((2R,4R,4aS,7aR)-4-amino-4a- | |
| | hydroxyoctahydrocyclopenta[b]pyran-2- | |
| | yl)pyridin-3-yl)-6-(2,6-difluorophenyl)-5- | |
| | fluoropicolinamide | |
| 218 | 4-amino-1-benzyl-N-(4-((2R,4R,5S,6R)-4,5- | |
| | dihydroxy-5,6-dimethyltetrahydro-2H-pyran- | |
| | 2-yl)pyridin-3-yl)-1H-pyrazole-3-carboxamide | |
| 219 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluorophenyl)-5- | |
| | fluoropicolinamide | |
| 220 | N-(4-((2S,4S,5R,6S)-4-amino-5-hydroxy-5,6- | |
| | dimethyltetrahydro-2H-pyran-2-yl)pyridin-3- | |
| | yl)-6-(2,6-difluorophenyl)-5- | |
| | fluoropicolinamide | |
| 221 | N-(4-((2R,4R,5S,6R)-4-amino-5-hydroxy-5,6- | |
| | dimethyltetrahydro-2H-pyran-2-yl)pyridin-3- | |
| | yl)-2-(2,6-difluorophenyl)thiazole-4- | |
| | carboxamide | |
| 222 | N-(4-((2S,4S,5R,6S)-4-amino-5-hydroxy-5,6- | |
| | dimethyltetrahydro-2H-pyran-2-yl)pyridin-3- | |
| | yl)-2-(2,6-difluorophenyl)thiazole-4- | |
| | carboxamide | |
| 223 | N-(4-((2S,4S,4aS,7aS)-4- | |
| | aminooctahydrocyclopenta[b]pyran-2- | |
| | yl)pyridin-3-yl)-6-(2,6-difluorophenyl)-5- | |
| | fluoropicolinamide | |
| 224 | N-(4-((2R,4R,4aR,7aR)-4- | |
| | aminooctahydrocyclopenta[b]pyran-2- | |
| | yl)pyridin-3-yl)-6-(2,6-difluorophenyl)-5- | |
| | fluoropicolinamide | |
| 225 | 6-(2,6-difluorophenyl)-N-(5-((2R,4R,5S,6R)- | |
| | 4,5-dihydroxy-6-(hydroxymethyl)tetrahydro- | |
| | 2H-pyran-2-yl)-2-methoxypyridin-4-yl)-5- | |
| | fluoropicolinamide | |
| 226 | 6-(2,6-difluorophenyl)-N-(5-((2R,4R,5S,6R)- | |
| | 4,5-dihydroxy-6-(hydroxymethyl)tetrahydro- | |
| | 2H-pyran-2-yl)-2-oxo-1,2-dihydropyridin-4- | |
| | yl)-5-fluoropicolinamide | |
| 227 | 3-amino-N-(4-((2S,4R,5R,6S)-4-amino-6-tert- | |
| | butyl-5-hydroxytetrahydro-2H-pyran-2- | |
| | yl)pyridin-3-yl)-6-phenylpyrazine-2- | |
| | carboxamide | |
| 228 | 3-amino-N-(4-((2R,4R,5S,6R)-4-amino-5- | |
| | hydroxy-5,6-dimethyltetrahydro-2H-pyran-2- | |
| | yl)pyridin-3-yl)-6-phenylpyrazine-2- | |
| | carboxamide | |
| 229 | 3-amino-N-(4-((2S,4S,5R,6S)-4-amino-5- | |
| | hydroxy-5,6-dimethyltetrahydro-2H-pyran-2- | |
| | yl)pyridin-3-yl)-6-phenylpyrazine-2- | |
| | carboxamide | |
| 230 | N-(4-((2S,4S,4aS,7aS)-4- | |
| | aminooctahydrocyclopenta[b]pyran-2- | |
| | yl)pyridin-3-yl)-2-(2,6- | |
| | difluorophenyl)thiazole-4-carboxamide | |
| 231 | N-(4-((2R,4R,4aR,7aR)-4- | |
| | aminooctahydrocyclopenta[b]pyran-2- | |
| | yl)pyridin-3-yl)-2-(2,6- | |
| | difluorophenyl)thiazole-4-carboxamide | |
| 232 | N-(4-((2R,4R,5S,6R)-4-amino-5-ethyl-5- | |
| | hydroxy-6-methyltetrahydro-2H-pyran-2- | |
| | yl)pyridin-3-yl)-6-(2,6-difluorophenyl)-5- | |
| | fluoropicolinamide | |
| 233 | 3-amino-N-(4-((2S,4S,4aS,7aS)-4- | 1H NMR (400 MHz, <cd3od>) |
| | aminooctahydrocyclopenta[b]pyran-2- | d ppm 1.38-1.75 (m, 7 H) |
| | yl)pyridin-3-yl)-6-phenylpyrazine-2- | 1.86 (q, J = 12.13 Hz, 1 H) 2.08 (dd, |
| | carboxamide | J = 11.74, 3.91 Hz, 2 H) 3.83 (dt, |
| | | J = 11.93, 4.99 Hz, 1 H) 4.19 (t, |
| | | J = 3.52 Hz, 1 H) 7.37-7.43 (m, |
| | | 1 H) 7.43-7.53 (m, 2 H) |
| | | 7.61 (d, J = 5.48 Hz, 1 H) 7.96 (d, |
| | | J = 7.43 Hz, 2 H) 8.47 (d, J = 5.09 Hz, |
| | | 1 H) 8.76 (s, 1 H) 9.14 (s, 1 |
| | | H) |
| 234 | N-(4-((2R,4R,5S,6R)-4-amino-5-ethyl-5- | |
| | hydroxy-6-methyltetrahydro-2H-pyran-2- | |
| | yl)pyridin-3-yl)-6-(2,6-difluorophenyl)-5- | |
| | fluoropicolinamide | |
| 235 | N-(4-((2R,4R,5S,6R)-4-amino-6-cyclopropyl- | |
| | 5-hydroxytetrahydro-2H-pyran-2-yl)pyridin-3- | |
| | yl)-6-(2,6-difluorophenyl)-5- | |
| | fluoropicolinamide | |
| 236 | N-(4-((2S,4S,5R,6S)-4-amino-6-cyclopropyl- | |
| | 5-hydroxytetrahydro-2H-pyran-2-yl)pyridin-3- | |
| | yl)-6-(2,6-difluorophenyl)-5- | |
| | fluoropicolinamide | |
| 237 | N-(4-((2R,4R,4aS,8aR)-4-amino-4a- | |
| | hydroxyoctahydro-2H-chromen-2-yl)pyridin- | |
| | 3-yl)-2-(2,6-difluorophenyl)pyrimidine-4- | |
| | carboxamide | |
| 238 | 3-amino-N-(4-((2R,4R,4aS,8aR)-4-amino-4a- | |
| | hydroxyoctahydro-2H-chromen-2-yl)pyridin- | |
| | 3-yl)-6-(2,6-difluorophenyl)-5- | |
| | fluoropicolinamide | |
| 239 | 5-amino-N-(4-((2R,4R,5S,6R)-4-amino-5- | |
| | ethyl-5-hydroxy-6-methyltetrahydro-2H- | |
| | pyran-2-yl)pyridin-3-yl)-2-(2,6- | |
| | difluorophenyl)pyrimidine-4-carboxamide | |
| 240 | 3-amino-N-(4-((2S,4S,5R,6S)-4-amino-5- | |
| | hydroxy-6-isopropyltetrahydro-2H-pyran-2- | |
| | yl)pyridin-3-yl)-6-phenylpyrazine-2- | |
| | carboxamide | |
| 241 | N-(4-((2R,4R,4aS,8aR)-4-amino-6- | |
| | oxooctahydro-2H-pyrano[3,2-b]pyridin-2- | |
| | yl)pyridin-3-yl)-6-(2,6-difluorophenyl)-5- | |
| | fluoropicolinamide | |
| 242 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-4-methoxyphenyl)-5- | |
| | fluoropicolinamide | |
| 243 | 3-amino-N-(4-((2S,4S,4aR,8aS)-4-amino-4a- | 1H NMR (400 MHz, <cd3od>) |
| | hydroxyoctahydro-2H-chromen-2-yl)pyridin- | d ppm 1.18-1.28 (m, 1 H) |
| | 3-yl)-6-phenylpyrazine-2-carboxamide | 1.34 (d, J = 12.91 Hz, 1 H) |
| | | 1.41-1.82 (m, 7 H) 1.95 (q, J = 12.52 Hz, 1 |
| | | H) 2.23-2.35 (m, 1 H) 3.52 (br. |
| | | s., 1 H) 4.94-5.03 (m, 1 H) |
| | | 7.35-7.43 (m, 1 H) |
| | | 7.45-7.52 (m, 2 H) 7.67 (d, J = 5.48 Hz, 1 |
| | | H) 7.99 (d, J = 7.43 Hz, 1 H) |
| | | 8.50 (d, J = 5.48 Hz, 1 H) |
| | | 8.77 (s, 1 H) 8.96-9.04 (m, 1 H) |
| 244 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-3-methoxyphenyl)-5- | |
| | fluoropicolinamide | |
| 245 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-4-methylphenyl)-5- | |
| | fluoropicolinamide | |
| 246 | 5-amino-N-(4-((2R,4R,5S,6R)-6-cyclopropyl- | |
| | 4,5-dihydroxy-5-methyltetrahydro-2H-pyran- | |
| | 2-yl)pyridin-3-yl)-2-(2,6- | |
| | difluorophenyl)pyrimidine-4-carboxamide | |
| 247 | N-(4-((2R,4R,5S,6R)-6-cyclopropyl-4,5- | |
| | dihydroxy-5-methyltetrahydro-2H-pyran-2- | |
| | yl)pyridin-3-yl)-6-(2,6-difluorophenyl)-5- | |
| | fluoropicolinamide | |
| 248 | N-(4-((2S,4S,5R,6S)-6-cyclopropyl-4,5- | |
| | dihydroxy-5-methyltetrahydro-2H-pyran-2- | |
| | yl)pyridin-3-yl)-6-(2,6-difluorophenyl)-5- | |
| | fluoropicolinamide | |
| 249 | 5-amino-N-(4-((2S,4S,5R,6S)-6-cyclopropyl- | |
| | 4,5-dihydroxy-5-methyltetrahydro-2H-pyran- | |
| | 2-yl)pyridin-3-yl)-2-(2,6- | |
| | difluorophenyl)pyrimidine-4-carboxamide | |
| 250 | 6-(2,6-difluorophenyl)-N-(4-((5S,7S,8R)-7,8- | |
| | dihydroxy-4-oxaspiro[2.5]octan-5-yl)pyridin- | |
| | 3-yl)-5-fluoropicolinamide | |
| 251 | 5-amino-2-(2,6-difluorophenyl)-N-(4- | |
| | ((5S,7S,8R)-7,8-dihydroxy-4- | |
| | oxaspiro[2.5]octan-5-yl)pyridin-3- | |
| | yl)pyrimidine-4-carboxamide | |
| 252 | 5-amino-2-(2,6-difluorophenyl)-N-(4- | |
| | ((5R,7R,8S)-7,8-dihydroxy-4- | |
| | oxaspiro[2.5]octan-5-yl)pyridin-3- | |
| | yl)pyrimidine-4-carboxamide | |
| 253 | 6-(2,6-difluorophenyl)-N-(4-((5R,7R,8S)-7,8- | |
| | dihydroxy-4-oxaspiro[2.5]octan-5-yl)pyridin- | |
| | 3-yl)-5-fluoropicolinamide | |
| 254 | N-(4-((2S,4S,5R,6S)-4-amino-6-(tert-butyl)-5- | |
| | hydroxytetrahydro-2H-pyran-2-yl)pyridin-3- | |
| | yl)-6-(2,6-difluorophenyl)-5- | |
| | fluoropicolinamide | |
| 255 | N-(4-((2R,4R,5S,6R)-4-amino-6-(tert-butyl)- | |
| | 5-hydroxytetrahydro-2H-pyran-2-yl)pyridin-3- | |
| | yl)-6-(2,6-difluorophenyl)-5- | |
| | fluoropicolinamide | |
| 256 | N-(4-((2R,4R,5S,6R)-4-amino-6-cyclopropyl- | |
| | 5-hydroxy-5-methyltetrahydro-2H-pyran-2- | |
| | yl)pyridin-3-yl)-6-(2,6-difluorophenyl)-5- | |
| | fluoropicolinamide | |
| 257 | N-(4-((2S,4S,5R,6S)-4-amino-6-cyclopropyl- | |
| | 5-hydroxy-5-methyltetrahydro-2H-pyran-2- | |
| | yl)pyridin-3-yl)-6-(2,6-difluorophenyl)-5- | |
| | fluoropicolinamide | |
| 258 | N-(4-((2R,4R,5S,6R)-4-amino-5-ethyl-5- | |
| | hydroxy-6-methyltetrahydro-2H-pyran-2- | |
| | yl)pyridin-3-yl)-6-(2,6-difluoro-4- | |
| | methoxyphenyl)-5-fluoropicolinamide | |
| 259 | N-(4-((2R,4R,5S,6R)-4-amino-5-ethyl-5- | |
| | hydroxy-6-methyltetrahydro-2H-pyran-2- | |
| | yl)pyridin-3-yl)-6-(2,6-difluoro-4- | |
| | methylphenyl)-5-fluoropicolinamide | |
| 260 | 5-amino-N-(4-((2R,4R,5S,6R)-4-amino-6-tert- | |
| | butyl-5-hydroxytetrahydro-2H-pyran-2- | |
| | yl)pyridin-3-yl)-2-(2,6- | |
| | difluorophenyl)pyrimidine-4-carboxamide | |
| 261 | 3-amino-N-(4-((2R,4R,5S,6R)-4-amino-6- | |
| | cyclopropyl-5-hydroxy-5-methyltetrahydro- | |
| | 2H-pyran-2-yl)pyridin-3-yl)-6-(2,6- | |
| | difluorophenyl)-5-fluoropicolinamide | |
| 262 | 5-amino-N-(4-((2R,4R,5S,6R)-4-amino-6- | |
| | cyclopropyl-5-hydroxy-5-methyltetrahydro- | |
| | 2H-pyran-2-yl)pyridin-3-yl)-2-(2,6- | |
| | difluorophenyl)pyrimidine-4-carboxamide | |
| 263 | N-(4-((2R,4R,5S,6R)-4-amino-6-cyclopropyl- | |
| | 5-hydroxy-5-methyltetrahydro-2H-pyran-2- | |
| | yl)pyridin-3-yl)-2-(2,6- | |
| | difluorophenyl)thiazole-4-carboxamide | |
| 264 | N-(4-((2R,4R,5S,6R)-4-amino-6-cyclopropyl- | |
| | 5-hydroxy-5-methyltetrahydro-2H-pyran-2- | |
| | yl)pyridin-3-yl)-6-(2,6-difluoro-4- | |
| | methylphenyl)-5-fluoropicolinamide | |
| 265 | N-(4-((2R,4R,5S,6R)-4-amino-6-cyclopropyl- | |
| | 5-hydroxy-5-methyltetrahydro-2H-pyran-2- | |
| | yl)pyridin-3-yl)-6-(2,6-difluoro-4- | |
| | methoxyphenyl)-5-fluoropicolinamide | |
| 266 | N-(4-((5S,7S,8R)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 267 | N-(4-((5R,7R,8S)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 268 | N-(4-((2R,4R,5S,6R)-4-amino-6-tert-butyl-5- | |
| | hydroxytetrahydro-2H-pyran-2-yl)pyridin-3- | |
| | yl)-2-(2,6-difluorophenyl)thiazole-4- | |
| | carboxamide | |
| 269 | N-(4-((2R,4R,5S,6R)-4-amino-6-tert-butyl-5- | |
| | hydroxytetrahydro-2H-pyran-2-yl)pyridin-3- | |
| | yl)-6-(2,6-difluoro-4-methoxyphenyl)-5- | |
| | fluoropicolinamide | |
| 270 | N-(4-((2R,4R,5S,6R)-4-amino-6-tert-butyl-5- | |
| | hydroxytetrahydro-2H-pyran-2-yl)pyridin-3- | |
| | yl)-6-(2,6-difluoro-4-methylphenyl)-5- | |
| | fluoropicolinamide | |
| 271 | N-(4-((2R,4R,5S,6R)-4-amino-5-hydroxy-6- | |
| | isopropyltetrahydro-2H-pyran-2-yl)pyridin-3- | |
| | yl)-6-(2,6-difluorophenyl)-5- | |
| | fluoropicolinamide | |
| 272 | 5-amino-N-(4-((2R,4R,5S,6R)-4-amino-5- | |
| | hydroxy-6-isopropyltetrahydro-2H-pyran-2- | |
| | yl)pyridin-3-yl)-2-(2,6- | |
| | difluorophenyl)pyrimidine-4-carboxamide | |
| 273 | 5-amino-N-(4-((2S,4S,5R,6R)-4-amino-5- | |
| | hydroxy-6-isopropyltetrahydro-2H-pyran-2- | |
| | yl)pyridin-3-yl)-2-(2,6- | |
| | difluorophenyl)pyrimidine-4-carboxamide | |
| 274 | N-(4-((2S,4S,5R,6R)-4-amino-5-hydroxy-6- | |
| | isopropyltetrahydro-2H-pyran-2-yl)pyridin-3- | |
| | yl)-6-(2,6-difluorophenyl)-5- | |
| | fluoropicolinamide | |
| 275 | N-(4-((2R,4R,5S,6R)-4-amino-6-ethyl-5- | |
| | hydroxy-5-methyltetrahydro-2H-pyran-2- | |
| | yl)pyridin-3-yl)-6-(2,6-difluorophenyl)-5- | |
| | fluoropicolinamide | |
| 276 | N-(4-((2S,4S,5R,6S)-4-amino-6-ethyl-5- | |
| | hydroxy-5-methyltetrahydro-2H-pyran-2- | |
| | yl)pyridin-3-yl)-6-(2,6-difluorophenyl)-5- | |
| | fluoropicolinamide | |
| 277 | N-(4-((2R,4R,5S,6R)-4-amino-6-(tert-butyl)- | |
| | 5-hydroxytetrahydro-2H-pyran-2-yl)pyridin-3- | |
| | yl)-2-(2,6-difluorophenyl)pyrimidine-4- | |
| | carboxamide | |
| 278 | N-(4-((2R,4S,5S,6R)-4-amino-6-(tert-butyl)-5- | |
| | hydroxytetrahydro-2H-pyran-2-yl)pyridin-3- | |
| | yl)-6-(2,6-difluorophenyl)-5- | |
| | fluoropicolinamide | |
| 279 | N-(4-((2S,4R,5R,6S)-4-amino-6-(tert-butyl)-5- | |
| | hydroxytetrahydro-2H-pyran-2-yl)pyridin-3- | |
| | yl)-6-(2,6-difluorophenyl)-5- | |
| | fluoropicolinamide | |
| 280 | N-(4-((2R,4R,5S,6R)-4-amino-6-(tert-butyl)- | |
| | 5-hydroxytetrahydro-2H-pyran-2-yl)pyridin-3- | |
| | yl)-6-(2,6-difluoro-4-(methylsulfonyl)phenyl)- | |
| | 5-fluoropicolinamide | |
| 281 | N-(4-((2S,4R,5S,6R)-4-amino-6-cyclopropyl- | (400 mHz, DMSO-d6) |
| | 5-hydroxy-5-methyltetrahydro-2H-pyran-2- | 0.14-0.13 (m, 4 H) |
| | yl)pyridin-3-yl)-6-(2,6-difluoro-4- | 0.16-0.31 (m, 1 H) 0.67-0.87 (m, 3H) |
| | (methylsulfonyl)phenyl)-5-fluoropicolinamide | 1.17 (s, 1 H) 1.46 (q, J = 12.13 Hz, |
| | | 1 H) 1.71 (d, J = 12.91 Hz, 1 |
| | | H) 1.84 (s, 1 H) 2.76 (d, J = 8.61 Hz, |
| | | 1 H) 3.35 (s, 3H) |
| | | 4.47-4.65 (m, 2 H) 7.27 (d, J = 4.70 Hz, |
| | | 1 H) 7.91 (d, J = 7.04 Hz, 2 |
| | | H) 8.24 (t, J = 9.00 Hz, 1 H) |
| | | 8.32 (d, J = 5.09 Hz, 1 H) 8.43 (dd, |
| | | J = 8.80, 4.11 Hz, 1 H) |
| | | 9.03-9.11 (m, 1 H) 10.41 (s, 1 H) |
| 282 | 6-(2,6-difluorophenyl)-5-fluoro-N-(4- | |
| | ((2R,4R,5S,6S)-6-(fluoromethyl)-4,5- | |
| | dihydroxytetrahydro-2H-pyran-2-yl)pyridin-3- | |
| | yl)picolinamide | |
| 283 | N-(4-((2S,4R,5S,6R)-4-amino-6-ethyl-5- | |
| | hydroxy-5-methyltetrahydro-2H-pyran-2- | |
| | yl)pyridin-3-yl)-6-(2,6-difluoro-4- | |
| | (methylsulfonyl)phenyl)-5-fluoropicolinamide | |
| 284 | N-(4-((2S,4R,5S,6R)-4-amino-5-ethyl-5- | |
| | hydroxy-6-methyltetrahydro-2H-pyran-2- | |
| | yl)pyridin-3-yl)-6-(2,6-difluoro-4- | |
| | (methylsulfonyl)phenyl)-5-fluoropicolinamide | |
| 285 | N-(4-((2R,4R,5S,6R)-4-amino-5-hydroxy-5,6- | 1H NMR (400 MHz, |
| | dimethyltetrahydro-2H-pyran-2-yl)pyridin-3- | <cd3od>) d 9.40 (s, 1H), |
| | yl)-6-(2,6-difluoro-4-(methylsulfonyl)phenyl)- | 8.55 (dd, J = 4.11, 8.80 Hz, 1H), |
| | 5-fluoropicolinamide | 8.37 (d, J = 5.09 Hz, 1H), |
| | | 8.11 (t, J = 8.80 Hz, 1H), 7.89 (d, J = 7.04 Hz, |
| | | 2H), 7.41 (d, J = 5.09 Hz, |
| | | 1H), 4.79 (dd, J = 1.96, |
| | | 11.74 Hz, 1H), 3.37 (s, 1H), |
| | | 3.30 (s, 3H), 2.89 (dd, J = 4.30, |
| | | 12.13 Hz, 1H), 2.00-2.06 (m, |
| | | 1H), 1.68-1.79 (m, 1H), |
| | | 1.31 (br. s., 1H), 0.87 (s, 3H), |
| | | 0.78 (d, J = 6.26 Hz, 3H) |
| 286 | 3-amino-N-(4-((2R,4S,5S,6R)-4-amino-6-tert- | |
| | butyl-5-hydroxytetrahydro-2H-pyran-2- | |
| | yl)pyridin-3-yl)-6-phenylpyrazine-2- | |
| | carboxamide | |
| 287 | N-(4-((5R,7S,8S)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 288 | N-(4-((5S,7R,8R)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 289 | N-(4-((5R,7R,8S)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 290 | N-(4-((5S,7S,8R)-7-amino-8-hydroxy-8- | |
| | methyl-4-oxaspiro[2.5]octan-5-yl)pyridin-3- | |
| | yl)-6-(2,6-difluorophenyl)-5- | |
| | fluoropicolinamide | |
| 291 | 3-amino-N-(4-((2R,4S,6S)-4-amino-6- | 1H NMR (400 MHz, <dmso>) |
| | isopropyltetrahydro-2H-pyran-2-yl)pyridin-3- | d ppm 0.60-0.71 (m, 3 H) |
| | yl)-6-phenylpyrazine-2-carboxamide | 0.75 (d, J = 6.65 Hz, 3 H) 1.16 (q, |
| | | J = 11.87 Hz, 1 H) 1.38 (q, |
| | | J = 11.74 Hz, 1 H) 1.58 (dq, |
| | | J = 13.25, 6.54 Hz, 1 H) 1.84 (d, |
| | | J = 11.74 Hz, 1 H) 2.12 (d, |
| | | J = 12.13 Hz, 1 H) 3.24 (dd, |
| | | J = 10.17, 6.65 Hz, 1 H) 3.35 (br. |
| | | s., 1 H) 4.77 (d, J = 10.56 Hz, 1 |
| | | H) 7.32-7.41 (m, 1 H) |
| | | 7.42-7.51 (m, 3 H) 7.66 (br. s., 1 H) |
| | | 7.86 (br. s., 2 H) 8.12 (d, J = 7.43 Hz, |
| | | 2 H) 8.49 (d, J = 5.09 Hz, 1 |
| | | H) 8.79-8.85 (m, 1 H) 8.89 (s, |
| | | 1 H) 10.33-10.46 (m, 1 H) |
| 292 | 3-amino-N-(4-((2S,4R,6R)-4-amino-6- | 1H NMR (400 MHz, <dmso>) |
| | isopropyltetrahydro-2H-pyran-2-yl)pyridin-3- | d ppm 0.64 (d, J = 6.65 Hz, 3 H) |
| | yl)-6-phenylpyrazine-2-carboxamide | 0.72 (d, J = 6.65 Hz, 3 H) |
| | | 1.12 (q, J = 11.74 Hz, 1 H) 1.34 (q, |
| | | J = 11.74 Hz, 1 H) 1.55 (dq, |
| | | J = 13.30, 6.65 Hz, 1 H) 1.80 (d, |
| | | J = 10.96 Hz, 1 H) 2.09 (d, |
| | | J = 11.74 Hz, 1 H) 3.21 (dd, |
| | | J = 10.17, 6.26 Hz, 1 H) 4.73 (d, |
| | | J = 10.56 Hz, 1 H) 7.31-7.38 (m, |
| | | 1 H) 7.39-7.47 (m, 3 H) |
| | | 7.63 (br. s., 1 H) 7.82 (br. s., 2 H) |
| | | 8.08 (d, J = 7.43 Hz, 2 H) |
| | | 8.46 (d, J = 5.09 Hz, 1 H) |
| | | 8.77-8.82 (m, 1 H) 8.86 (s, 1 H) 10.38 (s, |
| | | 1 H) |
| 293 | N-(4-((2R,4R,5S,6R)-4-amino-5-hydroxy-6- | |
| | isopropyltetrahydro-2H-pyran-2-yl)pyridin-3- | |
| | yl)-2-(2,6-difluorophenyl)pyrimidine-4- | |
| | carboxamide | |
| 295 | N-(4-((2R,4R,5S,6R)-4-amino-5-hydroxy-6- | |
| | isopropyltetrahydro-2H-pyran-2-yl)pyridin-3- | |
| | yl)-5-fluoro-6-(1H-pyrrolo[2,3-b]pyridin-5- | |
| | yl)picolinamide | |
| 296 | 3-amino-N-(4-((2R,4S)-4-amino-6,6- | |
| | dimethyltetrahydro-2H-pyran-2-yl)pyridin-3- | |
| | yl)-6-phenylpyrazine-2-carboxamide | |
| 297 | 3-amino-N-(4-((2S,4R)-4-amino-6,6- | |
| | dimethyltetrahydro-2H-pyran-2-yl)pyridin-3- | |
| | yl)-6-phenylpyrazine-2-carboxamide | |
| 298 | N-(4-((2R,4S)-4-amino-6,6- | |
| | dimethyltetrahydro-2H-pyran-2-yl)pyridin-3- | |
| | yl)-6-(2,6-difluorophenyl)-5- | |
| | fluoropicolinamide | |
| 299 | N-(4-((2S,4R)-4-amino-6,6- | |
| | dimethyltetrahydro-2H-pyran-2-yl)pyridin-3- | |
| | yl)-6-(2,6-difluorophenyl)-5- | |
| | fluoropicolinamide | |
| 300 | N-(4-((2R,4S,6S)-4-amino-6- | |
| | isopropyltetrahydro-2H-pyran-2-yl)pyridin-3- | |
| | yl)-6-(2,6-difluorophenyl)-5- | |
| | fluoropicolinamide | |
| 301 | N-(4-((2S,4R,6R)-4-amino-6- | |
| | isopropyltetrahydro-2H-pyran-2-yl)pyridin-3- | |
| | yl)-6-(2,6-difluorophenyl)-5- | |
| | fluoropicolinamide | |
| 302 | N-(4-((2R,4S,6S)-4-amino-6- | |
| | isopropyltetrahydro-2H-pyran-2-yl)pyridin-3- | |
| | yl)-6-(2,6-difluoro-4-(methylsulfonyl)phenyl)- | |
| | 5-fluoropicolinamide | |
| 303 | N-(4-((2S,4R,6R)-4-amino-6- | |
| | isopropyltetrahydro-2H-pyran-2-yl)pyridin-3- | |
| | yl)-6-(2,6-difluoro-4-(methylsulfonyl)phenyl)- | |
| | 5-fluoropicolinamide | |
| 304 | N-(4-((2R,4R,5S,6R)-4-amino-5-hydroxy-6- | |
| | isopropyltetrahydro-2H-pyran-2-yl)pyridin-3- | |
| | yl)-2-(4-oxopyridin-1(4H)-yl)pyrimidine-4- | |
| | carboxamide | |
| 305 | N-(4-((2S,4R,5S,6R)-4-amino-5-hydroxy-6- | |
| | isopropyltetrahydro-2H-pyran-2-yl)pyridin-3- | |
| | yl)-6-(2,6-difluoro-4-(methylsulfonyl)phenyl)- | |
| | 5-fluoropicolinamide | |
| 306 | N-(4-((2R,4R,5S,6R)-4-amino-5-hydroxy-6- | |
| | isopropyltetrahydro-2H-pyran-2-yl)pyridin-3- | |
| | yl)-5-fluoro-6-(4- | |
| | (methylsulfonyl)phenyl)picolinamide | |
| 307 | 6-(3-acetamido-2,6-difluorophenyl)-N-(4- | |
| | ((2S,4R,5S,6R)-4-amino-5-hydroxy-6- | |
| | isopropyltetrahydro-2H-pyran-2-yl)pyridin-3- | |
| | yl)-5-fluoropicolinamide | |
| 308 | N-(4-((2R,4R,5S,6R)-4-amino-5-hydroxy-6- | |
| | isopropyltetrahydro-2H-pyran-2-yl)pyridin-3- | |
| | yl)-6-(2,6-difluoro-3-isobutyramidophenyl)-5- | |
| | fluoropicolinamide | |
| 309 | N-(4-((2R,4R,5S,6R)-4-amino-5-hydroxy-6- | |
| | isopropyltetrahydro-2H-pyran-2-yl)pyridin-3- | |
| | yl)-5-fluoro-6-phenylpicolinamide | |
| 310 | N-(4-((2R,4R,5S,6R)-4-amino-5-hydroxy-6- | |
| | isopropyltetrahydro-2H-pyran-2-yl)pyridin-3- | |
| | yl)-2-(6-fluoro-2-oxopyridin-1(2H)- | |
| | yl)pyrimidine-4-carboxamide | |
| 311 | 6′-amino-N-(4-((2R,4R,5S,6R)-4-amino-5- | |
| | hydroxy-5,6-dimethyltetrahydro-2H-pyran-2- | |
| | yl)pyridin-3-yl)-2′,3-difluoro-2,3′-bipyridine- | |
| | 6-carboxamide | |
| 312 | N-(4-((2R,4R,5S,6R)-4-amino-5-hydroxy-6- | |
| | isopropyltetrahydro-2H-pyran-2-yl)pyridin-3- | |
| | yl)-5-fluoro-6-(3- | |
| | (methylsulfonyl)phenyl)picolinamide | |
| 313 | N-(4-((2S,4R)-4-amino-6,6- | |
| | dimethyltetrahydro-2H-pyran-2-yl)pyridin-3- | |
| | yl)-6-(2,6-difluoro-4-methylphenyl)-5- | |
| | fluoropicolinamide | |
| 314 | N-(4-((2S,4R)-4-amino-6,6- | |
| | dimethyltetrahydro-2H-pyran-2-yl)pyridin-3- | |
| | yl)-6-(2,6-difluoro-4-(2- | |
| | hydroxyethoxy)phenyl)-5-fluoropicolinamide | |
| 315 | N-(4-((2S,4R)-4-amino-6,6- | |
| | dimethyltetrahydro-2H-pyran-2-yl)pyridin-3- | |
| | yl)-6-(2,6-difluoro-4-(2- | |
| | methoxyethoxy)phenyl)-5-fluoropicolinamide | |
| 316 | N-(4-((2S,4R,5S,6R)-4-amino-5-hydroxy-6- | |
| | isopropyltetrahydro-2H-pyran-2-yl)pyridin-3- | |
| | yl)-6-(2,6-difluoro-3- | |
| | (methylcarbamoyl)phenyl)-5- | |
| | fluoropicolinamide | |
| 317 | N-(4-((2S,4R,5S,6R)-4-amino-5-hydroxy-6- | |
| | isopropyltetrahydro-2H-pyran-2-yl)pyridin-3- | |
| | yl)-6-(3-(dimethylcarbamoyl)-2,6- | |
| | difluorophenyl)-5-fluoropicolinamide | |
| 318 | N-(4-((2S,4R,5S,6R)-4-amino-5-hydroxy-6- | |
| | isopropyltetrahydro-2H-pyran-2-yl)pyridin-3- | |
| | yl)-6-(2,6-difluoro-4-(2- | |
| | methoxyethoxy)phenyl)-5-fluoropicolinamide | |
| 319 | N-(4-((2S,4R,5S,6R)-4-amino-5-hydroxy-6- | |
| | isopropyltetrahydro-2H-pyran-2-yl)pyridin-3- | |
| | yl)-6-(2,6-difluoro-4-(2- | |
| | hydroxyethoxy)phenyl)-5-fluoropicolinamide | |
| 320 | N-(4-((2R,4R,5S,6R)-4-amino-5-hydroxy-5,6- | |
| | dimethyltetrahydro-2H-pyran-2-yl)pyridin-3- | |
| | yl)-5-fluoro-6-phenylpicolinamide | |
| 321 | N-(4-((2R,4R,5S,6R)-4-amino-5-hydroxy-5,6- | |
| | dimethyltetrahydro-2H-pyran-2-yl)pyridin-3- | |
| | yl)-5-fluoro-6-(4- | |
| | (methylsulfonyl)phenyl)picolinamide | |
| 322 | N-(4-((2R,4R,5S,6R)-4-amino-6-cyclopropyl- | |
| | 5-hydroxytetrahydro-2H-pyran-2-yl)pyridin-3- | |
| | yl)-6-(2,6-difluoro-4-methylphenyl)-5- | |
| | fluoropicolinamide | |
| 323 | N-(4-((2R,4R,5S,6R)-4-amino-6-cyclopropyl- | |
| | 5-hydroxytetrahydro-2H-pyran-2-yl)pyridin-3- | |
| | yl)-6-(2,6-difluoro-4-methoxyphenyl)-5- | |
| | fluoropicolinamide | |
| 324 | N-(4-((2S,4S,5R,6S)-4-amino-6-cyclopropyl- | |
| | 5-hydroxytetrahydro-2H-pyran-2-yl)pyridin-3- | |
| | yl)-6-(2,6-difluoro-4-methylphenyl)-5- | |
| | fluoropicolinamide | |
| 325 | N-(4-((2S,4S,5R,6S)-4-amino-6-cyclopropyl- | |
| | 5-hydroxytetrahydro-2H-pyran-2-yl)pyridin-3- | |
| | yl)-6-(2,6-difluoro-4-methoxyphenyl)-5- | |
| | fluoropicolinamide | |
| 326 | N-(4-((2S,4S,5R,6S)-4-amino-6-cyclopropyl- | |
| | 5-hydroxytetrahydro-2H-pyran-2-yl)pyridin-3- | |
| | yl)-6-(2,6-difluoro-4-(methylsulfonyl)phenyl)- | |
| | 5-fluoropicolinamide | |
| 327 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-3-(methylthio)phenyl)-5- | |
| | fluoropicolinamide | |
| 328 | N-(4-((2S,4S,6S)-4-amino-6- | |
| | isopropyltetrahydro-2H-pyran-2-yl)pyridin-3- | |
| | yl)-2-(2,6-difluorophenyl)pyrimidine-4- | |
| | carboxamide | |
| 329 | N-(4-((2R,4R,5S,6R)-4-amino-5-hydroxy-6- | |
| | isopropyltetrahydro-2H-pyran-2-yl)pyridin-3- | |
| | yl)-6-(3-cyano-2,6-difluorophenyl)-5- | |
| | fluoropicolinamide | |
| 330 | N-(4-((2R,4R,5S,6R)-4-amino-5-hydroxy-6- | |
| | isopropyltetrahydro-2H-pyran-2-yl)pyridin-3- | |
| | yl)-6-(4-cyano-2-fluorophenyl)-5- | |
| | fluoropicolinamide | |
| 331 | N-(4-((2R,4R,5S,6R)-4-amino-6-ethyl-5- | |
| | hydroxytetrahydro-2H-pyran-2-yl)pyridin-3- | |
| | yl)-6-(2,6-difluorophenyl)-5- | |
| | fluoropicolinamide | |
| 332 | N-(4-((2S,4S,5R,6S)-4-amino-6-ethyl-5- | |
| | hydroxytetrahydro-2H-pyran-2-yl)pyridin-3- | |
| | yl)-6-(2,6-difluorophenyl)-5- | |
| | fluoropicolinamide | |
| 333 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-4- | |
| | (methylcarbamoyl)phenyl)-5- | |
| | fluoropicolinamide | |
| 334 | N-(4-((2R,4R,5S,6R)-4-amino-5-hydroxy-5,6- | |
| | dimethyltetrahydro-2H-pyran-2-yl)pyridin-3- | |
| | yl)-6-(4-(dimethylcarbamoyl)-2,6- | |
| | difluorophenyl)-5-fluoropicolinamide | |
| 335 | N-(4-((2R,4R,5S,6R)-4-amino-5-hydroxy-5,6- | |
| | dimethyltetrahydro-2H-pyran-2-yl)pyridin-3- | |
| | yl)-6-(1,1-dioxidothiomorpholino)-5- | |
| | fluoropicolinamide | |
| 336 | N-(4-((2R,4R,5S,6R)-4-amino-6-ethyl-5- | |
| | hydroxytetrahydro-2H-pyran-2-yl)pyridin-3- | |
| | yl)-6-(2,6-difluoro-4-(2-hydroxypropan-2- | |
| | yl)phenyl)-5-fluoropicolinamide | |
| 337 | N-(4-((2R,4S,6S)-4-amino-6- | |
| | isopropyltetrahydro-2H-pyran-2-yl)pyridin-3- | |
| | yl)-6-(3-cyano-2,6-difluorophenyl)-5- | |
| | fluoropicolinamide | |
| 338 | 6-(4-acetamido-2,6-difluorophenyl)-N-(4- | |
| | ((2R,4R,5S,6R)-4-amino-5-hydroxy-5,6- | |
| | dimethyltetrahydro-2H-pyran-2-yl)pyridin-3- | |
| | yl)-5-fluoropicolinamide | |
| 339 | N-(4-((2R,4R,5S,6R)-4-amino-5-hydroxy-5,6- | |
| | dimethyltetrahydro-2H-pyran-2-yl)pyridin-3- | |
| | yl)-6-(2,6-difluoro-4-isobutyramidophenyl)-5- | |
| | fluoropicolinamide | |
| 340 | 3-amino-N-(4-((2R,4R,5S,6R)-4,5-dihydroxy- | |
| | 6-propyltetrahydro-2H-pyran-2-yl)pyridin-3- | |
| | yl)-6-phenylpyrazine-2-carboxamide | |
| 341 | 3-amino-N-(4-((2R,4R,5S,6R)-4,5-dihydroxy- | |
| | 6-(hydroxymethyl)tetrahydro-2H-pyran-2- | |
| | yl)pyridin-3-yl)-6-phenylpyrazine-2- | |
| | carboxamide | |
| 342 | 3-amino-N-(4-((2S,4S,5R,6S)-4-amino-6- | |
| | ethyl-5-hydroxytetrahydro-2H-pyran-2- | |
| | yl)pyridin-3-yl)-6-phenylpyrazine-2- | |
| | carboxamide | |
| 343 | 3-amino-N-(4-((2R,4R,5S,6R)-4-amino-6- | |
| | ethyl-5-hydroxytetrahydro-2H-pyran-2- | |
| | yl)pyridin-3-yl)-6-phenylpyrazine-2- | |
| | carboxamide | |
| 344 | 3-amino-N-(4-((2R,4R,5S,6R)-4-amino-6- | |
| | cyclopropyl-5-hydroxy-5-methyltetrahydro- | |
| | 2H-pyran-2-yl)pyridin-3-yl)-6- | |
| | phenylpyrazine-2-carboxamide | |
| 345 | 3-amino-N-(4-((2S,4S,5R,6S)-4-amino-6- | |
| | ethyl-5-hydroxy-5-methyltetrahydro-2H- | |
| | pyran-2-yl)pyridin-3-yl)-6-phenylpyrazine-2- | |
| | carboxamide | |
| 346 | 3-amino-N-(4-((2R,4R,5S,6R)-4-amino-6- | |
| | cyclopropyl-5-hydroxytetrahydro-2H-pyran-2- | |
| | yl)pyridin-3-yl)-6-phenylpyrazine-2- | |
| | carboxamide | |
| 347 | 3-amino-N-(4-((2S,4S,5R,6S)-4-amino-6- | |
| | cyclopropyl-5-hydroxy-5-methyltetrahydro- | |
| | 2H-pyran-2-yl)pyridin-3-yl)-6- | |
| | phenylpyrazine-2-carboxamide | |
| 348 | 3-amino-N-(4-((2R,4R,5S,6R)-4-amino-6- | |
| | ethyl-5-hydroxy-5-methyltetrahydro-2H- | |
| | pyran-2-yl)pyridin-3-yl)-6-phenylpyrazine-2- | |
| | carboxamide | |
| 349 | 3-amino-N-(4-((2S,4S,5R,6S)-4-amino-6- | |
| | cyclopropyl-5-hydroxytetrahydro-2H-pyran-2- | |
| | yl)pyridin-3-yl)-6-phenylpyrazine-2- | |
| | carboxamide | |
| 350 | 3-amino-N-(4-((5S,7S,8R)-7-amino-8- | |
| | hydroxy-8-methyl-4-oxaspiro[2.5]octan-5- | |
| | yl)pyridin-3-yl)-6-phenylpyrazine-2- | |
| | carboxamide | |
| 351 | 3-amino-N-(4-((5R,7S,8S)-7-amino-8- | |
| | hydroxy-8-methyl-4-oxaspiro[2.5]octan-5- | |
| | yl)pyridin-3-yl)-6-phenylpyrazine-2- | |
| | carboxamide | |
| 352 | 3-amino-N-(4-((5S,7R,8R)-7-amino-8- | |
| | hydroxy-8-methyl-4-oxaspiro[2.5]octan-5- | |
| | yl)pyridin-3-yl)-6-phenylpyrazine-2- | |
| | carboxamide | |
| 353 | 3-amino-N-(4-((5R,7R,8S)-7-amino-8- | |
| | hydroxy-8-methyl-4-oxaspiro[2.5]octan-5- | |
| | yl)pyridin-3-yl)-6-phenylpyrazine-2- | |
| | carboxamide | |
| 354 | 3-amino-N-(4-((2R,4R,5S,6R)-4-amino-6-tert- | |
| | butyl-5-hydroxytetrahydro-2H-pyran-2- | |
| | yl)pyridin-3-yl)-6-phenylpyrazine-2- | |
| | carboxamide | |
| 355 | 3-amino-N-(4-((2S,4S,5R,6S)-4-amino-6-tert- | |
| | butyl-5-hydroxytetrahydro-2H-pyran-2- | |
| | yl)pyridin-3-yl)-6-phenylpyrazine-2- | |
| | carboxamide | |
| 356 | 3-amino-N-(4-((2R,4R,5S,6R)-4-amino-5- | |
| | hydroxy-6-isopropyltetrahydro-2H-pyran-2- | |
| | yl)pyridin-3-yl)-6-phenylpyrazine-2- | |
| | carboxamide | |

### KinaseGlo Pim1 ATP Depletion Assay

The activity of PIM1 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μA per well. To start the reaction, 10 μA of 5 nM Pim1 kinase and 80 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 40 μM ATP in assay buffer is added. Final assay concentrations are 2.5 nM PIM1, 20 μM ATP, 40 μM BAD peptide and 2.5% DMSO. The reaction is performed until approximately 50% of the ATP is depleted, then stopped with the addition of 20 μA KinaseGlo Plus (Promega Corporation) solution. The stopped reaction is incubated for 10 minutes and the remaining ATP detected via luminescence on the Victor2 (Perkin Elmer). Compounds of the foregoing examples were tested by the Pim1 ATP depletion assay and found to exhibit an IC₅₀ values as shown in TABLE 3 below. IC₅₀, the half maximal inhibitory concentration, represents the concentration of a test compound that is required for 50% inhibition of its target in vitro.

### KinaseGlo Pim2 ATP Depletion Assay

The activity of PIM2 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 piper well. To start the reaction, 10 μl of 10 nM Pim2 kinase and 20 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 8 μM ATP in assay buffer is added. Final assay concentrations are 5 nM PIM2, 4 μM ATP, 10 μM BAD peptide and 2.5% DMSO. The reaction is performed until approximately 50% of the ATP is depleted, then stopped with the addition of 20 μl KinaseGlo Plus (Promega Corporation) solution. The stopped reaction is incubated for 10 minutes and the remaining ATP detected via luminescence on the Victor2 (Perkin Elmer). Compounds of the foregoing examples were tested by the Pim2 ATP depletion assay and found to exhibit an IC₅₀ values as shown in TABLE 3 below.

### KinaseGlo Pim3 ATP Depletion Assay

The activity of PIM3 is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μl per well. To start the reaction, 10 μl of 10 nM Pim3 kinase and 200 μM BAD peptide (RSRHSSYPAGT-OH) in assay buffer (50 mM HEPES pH 7.5, 5 mM MgCl₂, 1 mM DTT, 0.05% BSA) is added into each well. After 15 minutes, 10 μl of 80 μM ATP in assay buffer is added. Final assay concentrations are 5 nM PIM1, 40 μM ATP, 100 μM BAD peptide and 2.5% DMSO. The reaction is performed until approximately 50% of the ATP is depleted, then stopped by the addition of 20 μl KinaseGlo Plus (Promega Corporation) solution. The stopped reaction is incubated for 10 minutes and the remaining ATP detected via luminescence on the Victor2 (Perkin Elmer). Compounds of the foregoing examples were tested by the Pim3 ATP depletion assay and found to exhibit an IC₅₀ values as shown in TABLE 3 below.

### KDR Kinase Inhibition Assay

LanthaScreen™ is the detection of Time-Resolved Fluorescence Resonance Energy Transfer (TR-FRET) using lanthanide chelates to measure interactions between various binding partners. The application of TR-FRET to assay kinase activity was first described by Mathis (1995). A TR-FRET assay was used to measure KDR kinase inhibitory activity. The assay panel was run on a Biomek FX liquid handling workstations. To the assay plates containing 50 nL compound or control solutions, 4.5 μL of buffer A (50 mM TRIS-HCl pH 7.4, 2 mM DTT, 0.02% Tween 20, 0.02 mM Na₃VO₄, H₂O nanpure) including a generic concentration of ATP (2 μM f.c.) was added per well, followed by 4.5 μL of buffer B (4 uM ATP in Buffer A) including a generic concentration of polyEAY (50 nM f.c.), KDR kinase, and divalent cations. Final concentration of kinase and cations were: [KDR kinase]=0.38 nM, [Mg]=10 mM, [Ca]=1 mM. After 1 hour of incubation the kinase reactions were stopped by the addition of 4.5 μL of stop solution D (50 mM EDTA, 20 mM TRIS-HCl pH 7.4, 0.04% NP-40) immediately followed by 4.5 μL of buffer A (50 mM TRIS-HCl pH 7.4, 2 mM DTT, 0.02% Tween 20, 0.02 mM Na₃VO₄, H₂O nanpure) including the Tb-labeled P-20 antibody to give a total detection volume of 18 μL. After an incubation time of 45 min in the dark, the plates were transferred into the Pherastar fluorescence reader for counting. The effect of compound on the enzymatic activity was obtained from the linear progress curves and determined from one reading (end point measurement). Compounds of the foregoing examples were tested by the KDR TR-FRET assay and found to exhibit an IC₅₀ values as shown in TABLE 3 and TABLE 4 below.

### PKCα and cABLT315 Kinase Caliper Assays

Assays were performed in 384 well microtiter plates. Each assay plate contained 8-point serial dilutions for test compounds, as well as two 16-point serial dilutions of staurosporine as reference compound, plus 16 high- and 16 low controls. Liquid handling and incubation steps were done on a Thermo CatX workstation equipped with a Innovadyne Nanodrop Express. Between pipetting steps, tips were cleaned in wash cycles using wash buffer. Plates with terminated kinase reactions were transferred to the Caliper LC3000 workstations for reading. Phosphorylated and unphosphorylated peptides were separated using the Caliper microfluidic mobilitishift technology and Kinase activities were calculated from the amounts of formed phospho-peptide.

Kinase reactions were prepared in 384 low volume plates by the following sequence:

1. 0.05 μA Compound (start with 1.8 mM in 90% DMSO/10% H₂O)

2. +4.5 μl 2× peptide/ATP solution

3. +4.5 μl 2× enzyme solution

4. Incubate for 60 min at 30° C.

4. +16 μl stop/run buffer

Independent of the kinase, all reactions were done performed in 50 mM HEPES, pH 7.5, 1 mM DTT, 0.02% Tween20, 0.02% BSA, and 0.6% DMSO. For cABLT315 assay specific details were as follows: [cABLT315 kinase]=2.4 nM, [ATP]=10 uM, [peptide]=2 uM, [Mg]=10 mM. For PKCα, assay specific details were as follows: [kinase]=0.012 nM, [ATP]=17 uM, [peptide]=1 uM, [Mg]=7 mM, [Ca]=0.2 mM. Compounds of the foregoing examples were tested by the PKCα and cABLT315 kinase Caliper assays and found to exhibit IC₅₀ values as shown in TABLE 3 and TABLE 4 below.

### GSK3β ATP Depletion Assay

The activity of GSK3β is measured using a luciferase-luciferin based ATP detection reagent to quantify ATP depletion resulting from kinase-catalyzed phosphoryl transfer to a peptide substrate. Compounds to be tested are dissolved in 100% DMSO and directly distributed into white 384-well plates at 0.5 μl per well. To start the reaction, 10 μl of 10 nM GSK3B kinase and 20 μM biotinylated CREB peptide (SGSGKRREILSRRP(pS)YR-NH2) in assay buffer (50 mM TRIS pH 7.5, 15 mM MgCl₂, 1 mM DTT, 0.1% BSA) is added into each well. After 15 minutes, 10 μl of 2 μM ATP in assay buffer is added. Final assay concentrations are 5 nM GSK3B, 2 μM ATP, 10 μM b-CREB peptide and 2.5% DMSO. The reaction is performed until approximately 50% of the ATP is depleted, then stopped with the addition of 20 μl KinaseGlo (Promega Corporation) solution. The stopped reaction is incubated for 10 minutes and the remaining ATP is detected via luminescence on the Victor2 (Perkin Elmer). Compounds of the foregoing examples were tested by the GSK3β ATP depletion assay and found to exhibit IC₅₀ values as shown in TABLE 3 and TABLE 4 below.

### Cell Proliferation Assay

KMS11 (human myeloma cell line), were cultured in IMDM supplemented with 10% FBS, sodium pyruvate and antibiotics. Cells were plated in the same medium at a density of 2000 cells per well into 96 well tissue culture plates, with outside wells vacant, on the day of assay. MMl.s (human myeloma cell line), were cultured in RPMI1640 supplemented with 10% FBS, sodium pyruvate and antibiotics. Cells were plated in the same medium at a density of 5000 cells per well into 96 well tissue culture plates, with outside wells vacant, on the day of assay.

Test compounds supplied in DMSO were diluted into DMSO at 500 times the desired final concentrations before dilution into culture media to 2 times final concentrations. Equal volumes of 2× compounds were added to the cells in 96 well plates and incubated at 37° C. for 3 days.

After 3 days plates were equilibrated to room temperature and equal volume of CellTiter-Glow Reagent (Promega) was added to the culture wells. The plates were agitated briefly and luminescent signal was measured with luminometer. The percent inhibition of the signal seen in cells treated with DMSO alone vs. cells treated with control compound was calculated and used to determine EC₅₀ values (i.e., the concentration of a test compound that is required to obtain 50% of the maximum effect in the cells) for tested compounds, as shown in TABLE 3 and TABLE 4 below.

### hERG Binding Assay

Compounds of the invention were pipetted into each well of pre-wet 96-well Millipore GF/C filter plates (#MSFCN6B50): 119 μl assay buffer, 1 μl test compound in 100% DMSO (or 100% DMSO only for total binding), 40 μl [3H] dofetilide (12.5 nM, final concentration 2.5 nM; Novartis radioisotope laboratory, East Hanover, N.J., USA, specific activity 15-45 Ci/mmol); 40 μl crude membrane suspension (ca. 15 μg protein). The final concentration of DMSO during the incubation was 0.5%. Incubations were performed at room temperature for 90 min. Non-specific binding (NSB) was defined as the binding remaining in the presence of 25 μM terfenadine (Sigma T9652). The incubations were terminated by rapid filtration on a Millipore filtration manifold, followed by three washes of 200 μA ice-cold assay buffer. The plates were left to dry overnight before adding 40 μA scintillant (MicroScint-20). The plates were then sealed (Sealing Tape SI, Nunc 236366) and read in a Wallac MicroBeta Trilux beta-counter for 1.5 min per well. Compounds were tested as 9-concentration response curves in duplicate, ranging from 30 μM to 3 nM in 1:3 dilution steps. Dilution curves were prepared in 100% DMSO. The reference compound (terfenadine) was tested as an eight-concentration response curve, ranging from 10 μM to 0.6 nM in 1:4 dilution steps. Compounds of the foregoing examples were tested by the hERG binding assay and found to exhibit IC₅₀ values as shown in TABLE 3 and TABLE 4 below.

The compounds of the invention are useful in vitro and/or in vivo in inhibiting the growth of cancer cells. The compounds may be used alone or in compositions together with a pharmaceutically acceptable carrier or excipient. Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), incorporated herein by reference.

Effective amounts of the compounds of the invention generally include any amount sufficient to detectably inhibit Pim activity by any of the assays described herein, by other Pim kinase activity assays known to those having ordinary skill in the art or by detecting an inhibition or alleviation of symptoms of cancer. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

The compounds of the present invention may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

While the compounds of the invention can be administered as the sole active pharmaceutical agent, they can also be used in combination with one or more other agents used in the treatment of cancer. The compounds of the present invention are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The compounds of the invention are also useful when co-administered with radiation therapy.

Therefore, in one embodiment of the invention, the compounds of the invention are also used in combination with known anticancer agents including, for example, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

In certain presently preferred embodiments of the invention, representative agents useful in combination with the compounds of the invention for the treatment of cancer include, for example, irinotecan, topotecan, gemcitabine, 5-fluorouracil, cytarabine, daunorubicin, PI3 Kinase inhibitors, mTOR inhibitors, DNA synthesis inhibitors, leucovorin carboplatin, cisplatin, taxanes, tezacitabine, cyclophosphamide, vinca alkaloids, imatinib (Gleevec), anthracyclines, rituximab, trastuzumab, as well as other cancer chemotherapeutic agents.

The above compounds to be employed in combination with the compounds of the invention will be used in therapeutic amounts as indicated in the Physicians' Desk Reference (PDR) 64th Edition (2010), which is incorporated herein by reference, or such therapeutically useful amounts as would be known to one of ordinary skill in the art.

The compounds of the invention and the other anticancer agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

In one embodiment, the invention provides a method of inhibiting Pim1, Pim2 or Pim3 in a human or animal subject. The method includes administering an effective amount of a compound, or a pharmaceutically acceptable salt thereof, of any of the embodiments of compounds of Formula I or II to a subject in need thereof.

The present invention will be understood more readily by reference to the following examples, which are provided by way of illustration and are not intended to be limiting of the present invention. Table 3 provides IC₅₀ values for the compounds in the different assays discussed above.

TABLE 3

| | | | | | | | | KMS11- | |
|---|---|---|---|---|---|---|---|---|---|
| | Pim1 | Pim2 | Pim3 | | KDR | PKC | | Luc | |
| | IC50 | IC50 | IC50 | GSK3b | IC50 | IC50 | cABLT315 | EC50 | HERGdof |
| Ex # | μM | μM | μM | IC50 μM | μM | μM | IC50 μM | μM | IC50μM |
| 1 | 0.017 | 0.005 | 0.0032 | 0.033 | | | | 9.35 | |
| 2 | 0.042 | 0.013 | 0.0083 | 0.051 | | | | >10 | |
| 3 | 0.0087 | 0.066 | 0.012 | 1.17 | | | | >10 | |
| 4 | 0.0011 | 0.004 | 0.0031 | 0.268 | | | | 7.24 | |
| 5 | 0.0259 | 0.254 | 0.0373 | 0.001 | 0.01 | 0.06 | 0.07 | 0.09 | >30 |
| 6 | 0.070 | 0.28 | 0.0536 | 0.720 | | | | >10 | |
| 7 | 0.008 | 0.151 | 0.017 | 0.879 | | | | >10 | |
| 8 | 0.051 | 0.323 | 0.149 | 1.33 | | | | >10 | |
| 9 | 0.0049 | 0.154 | 0.025 | 0.453 | | | | >10 | |
| 10 | 0.0186 | 0.040 | 0.0206 | 1.47 | | | | 8.63 | |
| 11 | 0.0007 | 0.0014 | 0.0018 | 0.718 | | | | 0.718 | |
| 12 | 0.001 | 0.005 | 0.003 | 0.220 | | | | 7.65 | |
| 13 | 0.001 | 0.012 | 0.005 | 0.583 | | | | 8.11 | |
| 14 | 0.001 | 0.002 | 0.003 | 0.070 | | | | 2.63 | |
| 15 | 0.001 | 0.008 | 0.002 | 0.001 | | | | 0.184 | |
| 16 | 0.001 | 0.003 | 0.002 | 0.101 | | | | 1.94 | >30 |
| 17 | 0.002 | 0.003 | 0.004 | 0.066 | | | | 4.16 | |
| 18 | 0.003 | 0.005 | 0.005 | 0.191 | | | | 5.71 | |
| 19 | 0.001 | 0.01 | 0.004 | 0.703 | | | | | |
| 20 | 0.008 | 0.103 | 0.032 | 2.565 | | | | | |
| 21 | 0.001 | 0.009 | 0.003 | 1.143 | | | | | |
| 22 | 0.02 | 0.16 | 0.009 | 0.87 | | | | >10 | |
| 23 | 0.006 | 0.041 | 0.008 | 0.2 | | | | >10 | |
| 24 | 0.001 | 0.007 | 0.004 | 0.177 | | | | 7.37 | |
| 25 | 0.001 | 0.002 | 0.002 | 0.66 | | | | 1.35 | |
| 26 | 0.004 | 0.029 | 0.002 | 0.277 | | | | >10 | |
| 27 | 0.001 | 0.001 | 0.001 | 0.25 | | | | | >30 |
| 28 | 0.002 | 0.007 | 0.003 | 0.022 | | | | 0.808 | |
| 29 | 0.001 | 0.001 | 0.001 | 0.041 | | | | 4.67 | |
| 30 | 0.001 | 0.001 | 0.001 | 0.201 | | | | 0.825 | |
| 31 | 0.001 | 0.001 | 0.001 | 0.117 | | | | 1.90 | |
| 32 | 0.005 | 0.044 | 0.003 | 0.454 | >10 | >10 | 9 | 1.12 | >30 |
| 33 | 0.001 | 0.002 | 0.002 | 0.276 | | | | 4.53 | |
| 34 | 0.022 | 0.17 | 0.038 | 0.061 | | | | 5.0 | |
| 35 | 0.001 | 0.001 | 0.001 | 0.088 | | | | 1.0 | |
| 36 | 0.001 | 0.008 | 0.002 | 0.001 | 0.001 | 0.1 | 0.03 | 0.2 | >30 |
| 37 | 0.012 | 0.065 | 0.017 | 0.014 | | | | >10 | |
| 38 | 0.001 | 0.003 | 0.002 | 0.015 | | | | 6.9 | |
| 39 | 0.004 | 0.04 | 0.004 | 0.267 | >10 | >10 | 8 | >10 | >30 |
| 40 | 0.001 | 0.002 | 0.002 | 0.002 | | | | 0.6 | |
| 41 | 0.001 | 0.065 | 0.007 | 0.017 | | | | 9.9 | |
| 42 | 0.001 | 0.003 | 0.001 | 0.023 | | | | 9.6 | |
| 43 | 0.001 | 0.001 | 0.001 | 0.188 | | | | 1.7 | |
| 44 | 0.001 | 0.001 | 0.001 | 0.339 | | | | 0.9 | |
| 45 | 0.068 | 1.268 | 0.338 | 4.307 | | | | | |
| 46 | 0.007 | 0.149 | 0.033 | 0.442 | | | | >10 | |
| 47 | 0.058 | 0.356 | 0.113 | 9.179 | | | | >10 | |
| 48 | 0.072 | 19.579 | 1.765 | 3.624 | | | | | |
| 49 | 0.027 | 1.418 | 0.073 | 0.21 | | | | | |
| 50 | 0.028 | 2.126 | 0.11 | 0.196 | | | | | |
| 51 | 0.054 | 7.358 | 0.617 | 3.722 | | | | | |
| 52 | 0.029 | 4.22 | 0.124 | 0.695 | | | | | |
| 53 | 0.078 | 10.269 | 0.847 | 3.116 | | | | | |
| 54 | 0.001 | 0.003 | 0.002 | 0.131 | | | | 2.1 | >30 |
| 55 | 0.002 | 0.101 | 0.026 | 0.911 | | | | 5.5 | |
| 56 | 0.001 | 0.003 | 0.002 | 0.073 | | | | 8.3 | >30 |
| 57 | 0.019 | 0.111 | 0.028 | 1.999 | | | | 6.1 | |
| 58 | 0.009 | 0.276 | 0.027 | 0.001 | 0.28 | 0.82 | 0.66 | 1.0 | >30 . |
| 59 | 0.006 | 0.162 | 0.014 | 0.757 | | | | >10 | |
| 60 | | 0.028 | | 0.092 | | | | >10 | >30 |
| 61 | 0.007 | 0.599 | 0.02 | 0.065 | | | | >10 | |
| 62 | 0.002 | 0.321 | 0.015 | 0.027 | | | | >10 | |
| 63 | 0.006 | 0.924 | 0.031 | 0.002 | 0.22 | 0.041 | 0.65 | 1.3 | >30 . |
| 64 | 0.0010 | 0.016 | 0.002 | 0.549 | | | | 0.8 | >30 |
| 65 | 0.0010 | 0.046 | 0.003 | 0.051 | | | | 8.3 | >30 |
| 66 | 0.0010 | 0.099 | 0.008 | 0.689 | | | | 2.2 | |
| 67 | 0.0040 | 0.3 | 0.022 | 0.984 | | | | >10 | |
| 68 | 0.0060 | 3.387 | 0.089 | 1.067 | | | | 6.0 | |
| 69 | 0.0040 | 0.497 | 0.053 | 1.802 | | | | 4.0 | |
| 70 | 0.0010 | 0.323 | 0.019 | 0.331 | | | | >10 | |
| 71 | 0.0010 | 0.052 | 0.006 | 1.48 | | | | 6.4 | >30 |
| 72 | 0.0010 | 0.015 | 0.006 | 0.385 | | | | 1.5 | |
| 73 | 0.0100 | 1.108 | 0.2 | 3.234 | | | | >10 | |
| 74 | 0.0010 | 0.011 | 0.005 | 0.316 | | | | 3.6 | >30 |
| 75 | 0.0010 | 0.004 | 0.002 | 0.228 | | | | 2.2 | |
| 76 | 0.0010 | 0.002 | 0.001 | 0.1 | | | | 0.2 | >30 . |
| 77 | 0.0500 | 0.756 | 0.236 | 4.895 | | | | 6.4 | |
| 78 | 0.1270 | 2.519 | 0.151 | 5.825 | | | | 1.5 | |
| 79 | 0.0020 | 0.017 | 0.003 | 1.446 | | | | >10 | |
| 80 | 0.0440 | 4.921 | 0.171 | 1.225 | | | | >10 | |
| 81 | 0.0310 | 0.72 | 0.044 | 0.854 | | | | >10 | |
| 82 | 0.0020 | 0.036 | 0.007 | 1.173 | | | | >10 | |
| 83 | 0.0190 | 0.291 | 0.032 | 2.798 | | | | >10 | |
| 84 | 0.0010 | 0.033 | 0.003 | 0.001 | 0.016 | 0.49 | 0.086 | 0.9 | >30 |
| 85 | 0.2210 | 20.643 | 0.564 | 0.257 | | | | >10 | >30 |
| 86 | 0.0050 | 0.164 | 0.015 | 0.003 | 0.3 | 0.79 | 0.7 | 2.8 | 25 |
| 87 | 0.9370 | >25 | 2.665 | 0.809 | | | | >10 | >30 |
| 88 | 0.0010 | 0.08 | 0.005 | 0.01 | | | | 9.7 | |
| 89 | 0.0010 | 0.006 | 0.002 | 0.088 | | | | 2.4 | |
| 90 | 0.0050 | 0.5 | 0.031 | 0.046 | | | | 4.0 | |
| 91 | 0.0030 | 0.047 | 0.013 | 0.171 | | | | 7.1 | |
| 92 | 0.0010 | 0.016 | 0.002 | 0.125 | | | | >10 | |
| 93 | 0.0010 | 0.059 | 0.005 | 0.117 | | | | >10 | |
| 94 | 0.0010 | 0.246 | 0.009 | 0.011 | | | | 4.2 | |
| 95 | 0.0080 | 2.168 | 0.08 | 0.032 | | | | >10 | |
| 96 | 0.0010 | 0.016 | 0.003 | 0.141 | | | | 3.9 | |
| 97 | 0.0050 | 0.263 | 0.037 | 0.313 | | | | >10 | |
| 98 | 0.0020 | 0.002 | 0.003 | 0.916 | | | | 0.5 | >30 |
| 99 | 0.0140 | 3.381 | 0.075 | 3.167 | | | | >10 | |
| 100 | 0.0100 | 0.799 | 0.042 | 2.469 | | | | >10 | |
| 101 | 0.0120 | 1.344 | 0.068 | 2.596 | | | | >10 | |
| 102 | 0.0280 | 2.886 | 0.103 | 2.003 | | | | >10 | |
| 103 | 0.0110 | 0.502 | 0.027 | 2.422 | | | | >10 | |
| 104 | 0.0020 | 0.003 | 0.002 | 0.979 | | | | 2.3 | >30 |
| 105 | 0.0010 | 0.05 | 0.006 | 1.181 | | | | 4.7 | |
| 106 | 0.0070 | 0.078 | 0.015 | 3.589 | | | | 9.2 | |
| 107 | 0.0020 | 0.02 | 0.004 | 0.196 | | | | 5.9 | |
| 108 | 0.0010 | 0.015 | 0.005 | 0.344 | | | | 4.2 | |
| 109 | 0.0680 | 3.421 | 0.088 | 0.272 | | | | >10 | |
| 110 | 0.0190 | 0.347 | 0.016 | 0.659 | | | | >10 | |
| 111 | 0.0010 | 0.043 | 0.002 | 0.026 | | | | >10 | |
| 112 | 0.0010 | 0.003 | 0.002 | 0.163 | | | | 1.2 | >30 |
| 113 | 0.0050 | 0.142 | 0.011 | 0.064 | | | | >10 | |
| 114 | 0.0040 | 0.144 | 0.014 | 0.652 | | | | 1.5 | |
| 115 | 0.0010 | 0.005 | 0.002 | 0.225 | | | | 1.3 | 22 |
| 116 | 0.0020 | 0.023 | 0.007 | 3.062 | | | | 1.9 | |
| 117 | 0.0080 | 2.477 | 0.04 | 2.73 | | | | 9.5 | |
| 118 | 0.0040 | 0.363 | 0.017 | 3.519 | | | | >10 | |
| 119 | 0.0010 | 0.011 | 0.002 | 0.629 | | | | 7.2 | |
| 120 | 0.0020 | 0.012 | 0.003 | 0.798 | | | | >10 | |
| 121 | 0.0090 | 0.795 | 0.044 | 3.229 | | | | 4.7 | 10 |
| 122 | 0.0020 | 0.045 | 0.01 | 12.347 | | | | 4.7 | 2 |
| 123 | 0.0010 | 0.044 | 0.007 | 0.421 | | | | 4.2 | 12 |
| 124 | 0.0010 | 0.005 | 0.002 | 3.405 | | | | 1.6 | 3 |
| 125 | 0.0100 | 2.323 | 0.165 | 0.104 | | | | >10 | |
| 126 | 0.0040 | 0.213 | 0.052 | 0.548 | | | | >10 | |
| 127 | 0.0010 | 0.324 | 0.019 | 0.025 | | | | 9.6 | >30 |
| 128 | 0.0010 | 0.028 | 0.006 | 0.276 | | | | 9.1 | >30 |
| 129 | 0.0020 | 0.147 | 0.013 | 0.361 | | | | >10 | >30 |
| 130 | 0.0120 | 2.345 | 0.084 | 0.228 | | | | >10 | |
| 131 | 0.0030 | 1.107 | 0.022 | 0.216 | | | | >10 | |
| 132 | 0.0060 | 0.465 | 0.033 | 0.196 | | | | >10 | |
| 133 | 0.0030 | 0.214 | 0.01 | 2.2 | | | | >10 | |
| 134 | 0.0100 | 0.678 | 0.4 | 0.759 | | | | >10 | |
| 135 | 0.0010 | 0.149 | 0.007 | 0.076 | | | | >10 | |
| 136 | 0.0180 | 0.363 | 0.013 | 0.165 | | | | >10 | |
| 137 | 0.003 | 0.023 | 0.005 | 0.351 | | | | >10 | |
| 138 | 0.024 | 0.027 | 0.014 | 0.235 | | | | >10 | |
| 139 | 0.001 | 0.005 | 0.002 | 0.450 | | | | >10 | |
| 140 | 0.018 | 0.054 | 0.019 | 2.25 | | | | >10 | |
| 141 | 0.181 | 0.720 | 0.094 | 3.36 | | | | >10 | |
| 142 | 1.08 | 6.6 | 0.890 | >25 | | | | >10 | |
| 143 | 0.0013 | 0.0035 | 0.0024 | 0.1015 | | >10 | >10 | 1.94 | >30 |
| 144 | 0.0053 | 0.0037 | 0.0043 | 0.3939 | | | | 4.34 | |
| 145 | 0.0184 | 0.0575 | 0.0194 | 2.2530 | | | | >10 | |
| 146 | 0.0057 | 0.0095 | 0.0051 | 0.3451 | | | | >10 | |
| 147 | 0.0174 | 0.0713 | 0.0185 | 1.3152 | | | | >10 | |
| 148 | 0.0297 | 0.0303 | 0.0204 | 0.6909 | | | | >10 | |
| 149 | 0.0798 | 0.4581 | 0.1431 | 0.2094 | | | | >10 | |
| 150 | 0.0571 | 0.1562 | 0.0533 | 0.2071 | | | | >10 | |
| 151 | 0.0052 | 0.0842 | 0.0251 | 1.4269 | | | | >10 | |
| 152 | 0.0013 | 0.0054 | 0.0023 | 0.4496 | | | | >10 | |
| 153 | 0.0236 | 0.0267 | 0.0135 | 0.2346 | | | | >10 | |
| 155 | 0.0040 | 0.0463 | 0.0053 | 0.3736 | | | | 4.92 | |
| 156 | 0.0254 | 0.5319 | 0.0424 | 0.0219 | | | | >10 | |
| 157 | 0.0080 | 0.0346 | 0.0081 | 0.0079 | | | | 9.63 | |
| 158 | 0.0088 | 0.0681 | 0.0069 | 0.0231 | | | | >10 | |
| 159 | 1.0800 | 6.6420 | 0.8903 | >25 | | | | >10 | |
| 160 | 0.1655 | 1.6417 | 0.2602 | 0.0674 | | | | >10 | |
| 161 | 0.0328 | 0.0223 | 0.0158 | 1.4314 | | | | >10 | |
| 162 | 0.2158 | 0.8185 | 0.1785 | 13.5471 | | | | >10 | |
| 164 | 0.0008 | 0.0019 | 0.0016 | 2.0663 | | | | 2.76 | |
| 165 | 0.0090 | 0.0677 | 0.0359 | 2.6765 | | | | >10 | |
| 166 | 0.0006 | 0.0012 | 0.0015 | 1.6791 | | | | 1.33 | |
| 167 | 0.0655 | 0.0920 | 0.0867 | 3.4058 | | | | >10 | |
| 168 | 0.0030 | 0.0023 | 0.0027 | 1.0301 | | | | 5.90 | |
| 169 | 0.0144 | 0.0093 | 0.0132 | 1.6596 | | | | >10 | |
| 170 | 1.4515 | 11.793 | 1.3188 | 3.5776 | | | | | |
| 171 | 8.3741 | >25 | 10.790 | >25 | | | | | |
| 172 | 0.0022 | 0.0031 | 0.0031 | 0.0210 | | | | | |
| 173 | 0.0022 | 0.0157 | 0.0036 | 0.0014 | | 0.31 | 0.07 | | >30 |
| 174 | 0.0005 | 0.0013 | 0.0016 | 0.0198 | | | | 2.63 | |
| 175 | 0.0357 | 0.0587 | 0.0574 | 0.5984 | | | | >10 | |
| 176 | 0.0638 | 1.8189 | 0.2017 | 0.0485 | | 2.60 | 7.40 | 3.60 | >30 |
| 177 | 0.0386 | 0.1707 | 0.1623 | 1.0587 | | | | 6.83 | |
| 178 | 0.1538 | 2.1750 | 0.1237 | 4.2477 | | >10 | >10 | >10 | >30 |
| 179 | 0.2588 | 3.0769 | 0.1320 | 2.6937 | | | | >10 | |
| 180 | 0.0084 | 0.0106 | 0.0072 | 0.1011 | | | | >10 | |
| 181 | 0.8249 | 9.0984 | 0.4920 | 4.2112 | | | | >10 | |
| 182 | 0.5392 | 2.7813 | 0.9510 | 2.6660 | | | | >10 | |
| 184 | >25 | >25 | >25 | >25 | | | | >10 | |
| 186 | 0.1593 | 3.1949 | 0.6103 | 0.0400 | | 0.76 | 9.90 | 6.91 | >30 |
| 188 | 0.0345 | 0.3177 | 0.1440 | 0.7308 | | | | >10 | |
| 190 | 0.0339 | 0.4943 | 0.0355 | 0.0385 | | | | >10 | |
| 191 | 1.7805 | >25 | 2.3741 | 1.0787 | | | | >10 | |
| 192 | >25 | >25 | >25 | >25 | | | | >10 | |
| 193 | 5.8504 | >25 | 7.5664 | >25 | | | | >10 | |
| 194 | 2.2078 | >25 | 2.8496 | 13.3129 | | | | >10 | |
| 195 | 0.0022 | 0.0121 | 0.0032 | 0.0010 | | 0.23 | 0.09 | 0.11 | >30 |
| 196 | 0.0034 | 0.0108 | 0.0036 | 0.6447 | | >10 | >10 | 8.10 | >30 |
| 198 | 0.0026 | 0.0536 | 0.0192 | 0.0240 | | | | >10 | |
| 200 | 0.0078 | 0.0467 | 0.0339 | 1.2044 | | | | >10 | |
| 201 | 0.1492 | 3.2122 | 0.1317 | 0.3831 | | | | >10 | |
| 202 | 0.7538 | 6.9139 | 1.0972 | 4.3972 | | | | >10 | |
| 203 | 6.6106 | >250 | 23.788 | 0.5432 | | | | >10 | |
| 204 | 0.0005 | 0.0028 | 0.0020 | 0.0462 | | | | 5.31 | |
| 205 | 0.0373 | 0.0454 | 0.0697 | 1.8660 | | | | >10 | |
| 206 | 0.0199 | 0.0338 | 0.0202 | 1.9236 | | | | >10 | |
| 207 | 0.0063 | 0.0060 | 0.0085 | 0.5716 | | | | 6.45 | |
| 208 | 0.0046 | 0.0088 | 0.0066 | 0.7957 | | | | >10 | |
| 209 | 0.0032 | 0.0079 | 0.0042 | 1.0648 | | | | 9.50 | |
| 210 | 0.0005 | 0.0024 | 0.0023 | 0.2895 | | >10 | >10 | 0.64 | 10 |
| 211 | 0.0010 | 0.0031 | 0.0022 | 0.6359 | | | | 2.70 | |
| 212 | 0.0179 | 0.5076 | 0.1550 | 11.5220 | | | | 3.36 | |
| 213 | 0.0039 | 0.0063 | 0.0058 | 0.5582 | | >10 | >10 | 2.80 | >30 |
| 214 | 0.2577 | 2.2483 | 0.3730 | >25 | | | | >10 | |
| 215 | 0.0006 | 0.0026 | 0.0040 | 0.2523 | | | | 0.80 | |
| 282 | 0.001 | 0.01 | 0.004 | 0.187 | | | | | |

Pim1, Pim2, Pim3 AlphaScreen Assay Pim 1, Pim 2 & Pim 3 AlphaScreen assays using high ATP (11-125×ATP Km) were used to determine the biochemical activity of the inhibitors. The activity of Pim 1, Pim 2, & Pim 3 is measured using a homogeneous bead based system quantifying the amount of phosphorylated peptide substrate resulting from kinase-catalyzed phosphoryl transfer to a peptide substrate. Compounds to be tested are dissolved in 100% DMSO and directly distributed to a white 384-well plate at 0.25 μA per well. To start the reaction, 5 μl of 100 nM Bad peptide (Biotin-AGAGRSRHSSYPAGT —OH) and ATP (concentrations described below) in assay buffer (50 mM Hepes, pH=7.5, 5 mM MgCl₂, 0.05% BSA, 0.01% Tween-20, 1 mM DTT) is added to each well. This is followed by the addition of 5 μl/well of Pim 1, Pim 2 or Pim 3 kinase in assay buffer (concentrations described below). Final assay concentrations (described below) are in 2.5% DMSO. The reactions are performed for ˜2 hours, then stopped by the addition of 10 μl of 0.75 μg/ml anti-phospho Ser/Thr antibody (Cell Signaling), 10 μg/ml Protein A AlphaScreen beads (Perkin Elmer), and 10 μg/ml streptavidin coated AlphaScreen beads in stop/detection buffer (50 mM EDTA, 95 mM Tris, pH=7.5, 0.01% Tween-20). The stopped reactions are incubated overnight in the dark. The phosphorylated peptide is detected via an oxygen anion initiated chemiluminescence/fluorescence cascade using the Envision plate reader (Perkin Elmer).

AlphaScreen Assay Conditions

| Enzyme | Enzyme conc. | b-BAD peptide | ATP conc. | ATP Km |
|---|---|---|---|---|
| source | (nM) | conc. (nM) | (uM) | (app) (uM) |
| Pim 1 (INV) | 0.0025 | 50 | 2800 | 246 |
| Pim 2 (INV) | 0.01 | 50 | 500 | 4 |
| Pim 3 (NVS) | 0.005 | 50 | 2500 | 50 |

Indicated compounds of the foregoing examples were tested by the Pim 1, Pim 2 & Pim 3 AlphaScreen assays and found to exhibit an IC₅₀ values as shown in Table 4, below. IC₅₀, the half maximal inhibitory concentration, represents the concentration of a test compound that is required for 50% inhibition of its target in vitro under the described assay conditions.

Using the procedures of Cell Proliferation Assay, the EC₅₀ concentration of indicated compounds of the examples in were determined in KMS11 cells as shown in Table 4.

TABLE 4

| | Pim1 | Pim2 | Pim3 | GSK3b | KDR | PKC | | KMS11- | |
|---|---|---|---|---|---|---|---|---|---|
| | IC50 | IC50 | IC50 | IC50 | IC50 | IC50 | cABLT315 | Luc EC50 | HERGdof |
| Ex # | μM | μM | μM | μM | μM | μM | IC50 μM | μM | IC50 μM |
| 163 | | 7.49 | | | | | | | |
| 183 | | 0.0766 | | 0.0044 | 0.0068 | 0.0340 | | 1.57 | |
| 185 | | 6.4296 | | 0.6700 | 2.0000 | 0.6500 | | 9.39 | |
| 187 | | 0.0863 | | 0.0033 | 0.0420 | 0.0500 | | 0.95 | |
| 189 | | 24.972 | | 0.0710 | 1.4000 | 0.7100 | | 8.83 | |
| 197 | | 0.0892 | | 0.0080 | 0.0400 | 0.0410 | | 0.07 | |
| 199 | | 0.1015 | | 0.0076 | 0.0620 | 0.0310 | | 0.59 | |
| 216 | 0.00014 | | 0.0017 | 0.9455 | | | | 0.53 | |
| 217 | 0.00517 | 0.106 | 0.0512 | 1.3914 | | | | 5.33 | |
| 218 | 12.5433 | >25 | >25 | 1.2944 | | | | >10 | |
| 219 | 0.00009 | 0.0085 | 0.0014 | 0.9785 | | >10 | >10 | 0.42 | 16 |
| 220 | 0.00209 | 0.2275 | 0.0299 | 9.1408 | | | | 6.21 | |
| 221 | 0.00064 | | 0.0187 | 0.0746 | | | | 7.72 | |
| 222 | 0.01030 | | 0.1890 | 3.0623 | | | | >10 | |
| 223 | 0.00462 | | 0.2010 | 9.8328 | | | | 4.38 | |
| 224 | 0.00010 | | 0.0041 | 0.3628 | | >10 | >10 | 0.65 | 2 |
| 225 | 14.6123 | | >25 | >25 | | | | >10 | |
| 226 | 1.43023 | | 23.534 | 10.637 | | | | >10 | |
| 227 | | 0.5495 | | 0.0064 | 1.7 | 0.055 | | 5.31 | |
| 228 | | 0.1203 | | 0.0130 | 0.038 | 0.076 | | 2.06 | |
| 229 | | 3.3087 | | 0.0390 | 0.79 | 0.37 | | 5.87 | |
| 230 | 0.00683 | | 0.4818 | 4.5774 | | 1.10 | >10 | 2.99 | 4 |
| 231 | 0.00024 | | 0.0146 | 0.0532 | | | | 5.82 | |
| 232 | 0.00008 | 0.0067 | 0.0014 | 0.6216 | | 9.50 | >10 | 0.05 | 5 |
| 233 | | 5.1395 | | 0.3300 | 3.0000 | .5500 | | 0.10 | |
| 234 | 0.00935 | 0.817 | 0.1956 | 20.792 | | | | 7.53 | |
| 235 | 0.00035 | 0.0099 | 0.0076 | 7.4235 | | | | | |
| 236 | 0.00367 | 0.209 | 0.1159 | >25 | | | | | |
| 237 | 0.00337 | 0.070 | 0.0240 | 0.0130 | | | | | |
| 238 | 0.00008 | 0.0018 | 0.0006 | 0.3926 | | >10 | >10 | | 4 |
| 239 | 0.00092 | 0.024 | 0.0084 | 0.3517 | | | | | |
| 240 | | 2.7692 | | 0.2700 | 5.1000 | 0.2900 | | 0.38 | |
| 241 | 0.02076 | 0.3929 | 0.1324 | 6.3001 | | | | | |
| 242 | 0.00004 | 0.0099 | 0.0008 | 0.4707 | | >10 | >10 | | 6 |
| 243 | | | | 0.8700 | 8.4000 | 7.2000 | | | |
| 244 | 0.00008 | 0.0122 | 0.0012 | 0.8094 | | | | | |
| 245 | 0.00005 | 0.0069 | 0.0008 | 0.7679 | | | | | |
| 246 | 0.07120 | 0.8177 | 0.9699 | 0.9826 | | | | | |
| 247 | 0.00401 | 0.1852 | 0.1026 | 1.1981 | | | | | |
| 248 | 0.09201 | 9.2793 | 2.2665 | 1.9667 | | | | | |
| 249 | 0.98884 | >25 | 20.871 | 6.0151 | | | | | |
| 250 | 0.02009 | 3.5865 | 0.4990 | 1.0444 | | | | | |
| 251 | 0.09068 | 9.1031 | 1.3188 | 1.1114 | | | | | |
| 252 | 0.02028 | 0.8484 | 0.2524 | 0.5718 | | | | | |
| 253 | 0.00099 | 0.0795 | 0.0126 | 0.2725 | | | | | |
| 254 | 0.00078 | 0.0472 | 0.0160 | 4.6950 | | | | | |
| 255 | 0.00003 | 0.0017 | 0.0004 | 2.1926 | | | | | |
| 256 | 0.00017 | 0.0061 | 0.0017 | 4.6245 | | | | | |
| 257 | 0.00188 | 0.1727 | 0.0359 | 19.980 | | | | | |
| 258 | 0.00004 | 0.0062 | 0.0005 | 0.1832 | | | | | |
| 259 | 0.00003 | 0.0048 | 0.0004 | 0.2179 | | | | | |
| 260 | 0.00034 | 0.0060 | 0.0036 | 1.0443 | | | | | |
| 261 | 0.00011 | 0.0061 | 0.0012 | 5.5200 | | | | | |
| 262 | 0.00215 | 0.0182 | 0.0181 | 2.0208 | | | | | |
| 263 | 0.00126 | 0.0727 | 0.0231 | 0.3338 | | | | | |
| 264 | 0.00011 | 0.0081 | 0.0024 | 1.7908 | | | | | |
| 265 | 0.00013 | 0.0096 | 0.0023 | 1.0466 | | | | | |
| 266 | 0.00198 | 0.3937 | 0.0275 | 6.0566 | | | | | |
| 267 | 0.00252 | 0.2133 | 0.0297 | 6.9266 | | | | | |
| 268 | 0.00016 | 0.0203 | 0.0036 | 0.2067 | | | | | |
| 269 | 0.00003 | 0.0024 | 0.0005 | 0.6129 | | | | | |
| 270 | 0.00003 | 0.0031 | 0.0005 | 1.0565 | | | | | |
| 271 | 0.00015 | 0.0043 | 0.0021 | 2.7527 | | | | | |
| 272 | 0.00066 | 0.0109 | 0.0090 | 1.2244 | | | | | |
| 273 | 0.02311 | 0.6604 | 0.5125 | 16.072 | | | | | |
| 274 | 0.00470 | 0.3053 | 0.0957 | 10.684 | | | | | |
| 275 | 0.00262 | 0.2337 | 0.0519 | 16.578 | | | | | |
| 276 | 0.00047 | 0.0174 | 0.0046 | 20.860 | | | | | |
| 277 | 0.00105 | 0.0275 | 0.0227 | 0.4993 | | | | | |
| 278 | | 0.0707 | | 9.7025 | | | | | |
| 279 | | 0.0271 | | 0.5483 | | | | | |
| 280 | | 0.0118 | | 2.2478 | | | | 0.20 | |
| 281 | | 0.0623 | | 4.2468 | | | | 1.09 | |
| 283 | | 0.0874 | | 12.511 | | | | | |
| 284 | | 0.0591 | | 0.6194 | | | | | |
| 285 | | 0.0449 | | 1.2917 | | | | 1.17 | |
| 286 | | 0.0649 | | 0.0005 | 0.1800 | | | 1.97 | |
| 287 | | 0.3877 | | 1.5011 | | | | | |
| 288 | | 0.2724 | | 16.334 | | | | | |
| 289 | 0.0025 | 0.2133 | 0.0297 | 6.9266 | | | | | |
| 290 | 0.00198 | 0.3937 | 0.0275 | 6.0566 | | | | | |
| 291 | | 0.0193 | | 0.0044 | 0.34 | 0.043 | | 0.34 | |
| 292 | | 0.7642 | | 0.0440 | 4.3 | 0.1 | | 0.70 | |
| 293 | | 0.1535 | | 0.9497 | | | | | |
| 294 | | 0.0102 | | >25 | | | | 0.16 | |
| 295 | | 0.3449 | | 4.3010 | | | | | |
| 296 | | 0.2830 | | 0.0094 | | 0.04 | 0.11 | | |
| 297 | | 0.5142 | | 0.0079 | | 0.01 | 0.86 | | |
| 298 | | 0.2338 | | 4.9443 | | | | | |
| 299 | | 0.0064 | | 0.2251 | | | | 0.14 | |
| 300 | | 0.0115 | | 3.2973 | | | | 0.69 | |
| 301 | | 0.1595 | | 20.730 | | | | | |
| 302 | | 0.0713 | | 1.5827 | | | | 1.62 | |
| 303 | | 1.2467 | | 10.337 | | | | | |
| 304 | | >25 | | >25 | | | | | |
| 305 | | 0.0153 | | 2.8367 | | | | 0.31 | |
| 306 | | 1.6616 | | 2.8227 | | | | | |
| 307 | | 0.0501 | | 3.4163 | | | | | |
| 308 | | 0.0685 | | 18.291 | | | | | |
| 309 | | 0.1307 | | 8.1227 | | | | | |
| 310 | | 6.9384 | | 3.3149 | | | | | |
| 311 | | 0.0846 | | 2.2966 | | | | | |
| 312 | | 2.48 | | >25 | | | | | |
| 313 | 0.00006 | 0.0086 | 0.0014 | 0.1278 | | | | 0.36 | |
| 314 | 0.00005 | 0.0075 | 0.0011 | 0.1088 | | | | 0.06 | |
| 315 | 0.00007 | 0.0117 | 0.0015 | 0.1209 | | | | 0.20 | |
| 316 | 0.00027 | 0.0051 | .00576 | 4.3879 | | | | 0.06 | |
| 317 | 0.0010 | 0.0066 | 0.0096 | >25 | | | | 0.08 | |
| 318 | 0.00005 | 0.0023 | 0.0017 | 1.1596 | | | | 0.08 | |
| 319 | 0.00007 | 0.0035 | 0.0018 | 1.5739 | | | | 0.03 | |
| 320 | 0.0010 | 0.2448 | 0.042 | 3.2507 | | | | 4.67 | |
| 321 | 0.0047 | 1.3670 | 0.783 | 1.8819 | | | | >10 | |
| 322 | 0.0003 | 0.0067 | 0.009 | 5.2367 | | | | 0.23 | |
| 323 | 0.0003 | 0.0134 | 0.0097 | 4.1961 | | | | 0.19 | |
| 324 | 0.0034 | 0.3964 | 0.184 | >25 | | | | 4.32 | |
| 325 | 0.004 | 0.5037 | 0.181 | >25 | | | | 5.47 | |
| 326 | 0.0275 | 2.1997 | 1.93 | >25 | | | | >10 | |
| 327 | 0.00007 | 0.0188 | 0.0026 | 0.6401 | | | | 0.31 | |
| 328 | 0.007 | 0.1651 | 0.172 | | | | | 0.54 | |
| 329 | 0.0007 | 0.0147 | 0.0147 | | | | | 0.19 | |
| 330 | 0.002 | 0.2286 | 0.232 | | | | | 2.43 | |
| 331 | 0.0002 | 0.0118 | 0.006 | | | | | 0.07 | |
| 332 | 0.0063 | 0.3098 | 0.157 | | | | | 4.20 | |
| 333 | | 0.0273 | | | | | | 3.76 | |
| 334 | | 0.0627 | | | | | | 0.76 | |
| 335 | | >25 | | | | | | >10 | |
| 336 | | 0.0039 | | | | | | 0.18 | |
| 337 | | 0.0400 | | | | | | 1.35 | |
| 338 | | 0.0021 | | | | | | 0.94 | |
| 339 | | 0.0035 | | | | | | 0.23 | |
| 340 | | 3.6467 | | 0.0009 | 0.1300 | 0.8600 | | 0.99 | |
| 341 | | >25 | | 0.0030 | 0.0600 | 0.6500 | | 10.00 | |
| 342 | | 1.3704 | | 0.0390 | 0.5100 | 0.0710 | | 0.77 | |
| 343 | | 0.0491 | | 0.0120 | 0.0130 | 0.0230 | | 0.15 | |
| 344 | | 0.1063 | | 0.0290 | 0.0580 | 0.1100 | | 0.87 | |
| 345 | | 7.8089 | | 0.3700 | 4.4000 | 0.6600 | | 3.17 | |
| 346 | | 0.0627 | | 0.0092 | 0.0280 | 0.0600 | | 0.47 | |
| 347 | | 8.0169 | | 0.5700 | 3.7000 | 1.0000 | | 2.43 | |
| 348 | | 0.1460 | | 0.1200 | 0.1500 | 0.2200 | | 1.19 | |
| 349 | | 2.7386 | | 0.1000 | 2.3000 | 0.1400 | | 0.54 | |
| 350 | | 0.7861 | | 0.0078 | 0.2200 | 0.0210 | | | |
| 351 | | 0.5377 | | 0.0004 | 0.007 | | | | |
| 352 | | 1.9755 | | 0.0053 | 0.0280 | 0.0550 | | | |
| 353 | | 2.4303 | | 0.0250 | 0.2500 | 0.0840 | | | |
| 354 | | 0.0090 | | 0.0032 | 0.0620 | 0.0220 | | 0.14 | |
| 355 | | 8.7478 | | 0.1700 | 6.4000 | 0.8400 | | 0.21 | |
| 356 | | 0.0281 | | 0.0086 | 0.0430 | 0.0750 | | 0.18 | |

### FGFR3 Kinase Inhibition Assay

LanthaScreen™ is the detection of Time-Resolved Fluorescence Resonance Energy Transfer (TR-FRET) using lanthanide chelates to measure interactions between various binding partners. The application of TR-FRET to assay kinase activity was first described by Mathis (1995). A TR-FRET assay was used to measure FGFR3 kinase inhibitory activity. The assay panel was run on a Biomek FX liquid handling workstations. To the assay plates containing 50 mL compound or control solutions, 4.5 μL of buffer A (50 mM TRIS-HCl pH 7.4, 2 mM DTT, 0.02% Tween 20, 0.02 mM Na₃VO₄, H₂O nanpure) including a generic concentration of ATP (2 μM f.c.) was added per well, followed by 4.5 μL of buffer B (4 uM ATP in Buffer A) including a generic concentration of polyEAY (50 nM f.c.), FGFR3 kinase, and divalent cations. Final concentration of kinase and cations were: [FGFR3 kinase]=0.20 nM, [Mg]=3 mM, [Mn]=3 mM. After 1 hour of incubation the kinase reactions were stopped by the addition of 4.5 μL of stop solution D (50 mM EDTA, 20 mM TRIS-HCl pH 7.4, 0.04% NP-40) immediately followed by 4.5 μL of buffer A (50 mM TRIS-HCl pH 7.4, 2 mM DTT, 0.02% Tween 20, 0.02 mM Na₃VO₄, H₂O nanpure) including the Tb-labeled P-20 antibody to give a total detection volume of 18 μL. After an incubation time of 45 min in the dark, the plates were transferred into the Pherastar fluorescence reader for counting. The effect of compound on the enzymatic activity was obtained from the linear progress curves and determined from one reading (end point measurement). Compounds of the foregoing examples were tested by the FGFR3TR-FRET assay and found to exhibit an IC₅₀ values as shown in TABLE 5 below.

### PDGFRaV561D Kinase Inhibition Assay

LanthaScreen™ is the detection of Time-Resolved Fluorescence Resonance Energy Transfer (TR-FRET) using lanthanide chelates to measure interactions between various binding partners. The application of TR-FRET to assay kinase activity was first described by Mathis (1995). A TR-FRET assay was used to measure PDGFRaV561D kinase inhibitory activity. The assay panel was run on a Biomek FX liquid handling workstations. To the assay plates containing 50 mL compound or control solutions, 4.5 μL of buffer A (50 mM TRIS-HCl pH 7.4, 2 mM DTT, 0.02% Tween 20, 0.02 mM Na₃VO₄, H₂O nanpure) including a generic concentration of ATP (2 μM f.c.) was added per well, followed by 4.54 of buffer B (4 uM ATP in Buffer A) including a generic concentration of polyEAY (50 nM f.c.), PDGFRaV561D kinase, and divalent cations. Final concentration of kinase and cations were: [PDGFRaV561D kinase]=4.4 nM, [Mn]=10 mM. After 1 hour of incubation the kinase reactions were stopped by the addition of 4.5 μL of stop solution D (50 mM EDTA, 20 mM TRIS-HCl pH 7.4, 0.04% NP-40) immediately followed by 4.5 μL of buffer A (50 mM TRIS-HCl pH 7.4, 2 mM DTT, 0.02% Tween 20, 0.02 mM Na₃VO₄, H₂O nanpure) including the Tb-labeled P-20 antibody to give a total detection volume of 184. After an incubation time of 45 min in the dark, the plates were transferred into the Pherastar fluorescence reader for counting. The effect of compound on the enzymatic activity was obtained from the linear progress curves and determined from one reading (end point measurement). Compounds of the foregoing examples were tested by the PDGFRaV561D TR-FRET assay and found to exhibit an IC₅₀ values as shown in TABLE 5 below.

### FLT3D835Y Kinase Inhibition Assay

LanthaScreen™ is the detection of Time-Resolved Fluorescence Resonance Energy Transfer (TR-FRET) using lanthanide chelates to measure interactions between various binding partners. The application of TR-FRET to assay kinase activity was first described by Mathis (1995). A TR-FRET assay was used to measure FLT3D835Y kinase inhibitory activity. The assay panel was run on a Biomek FX liquid handling workstations. To the assay plates containing 50 mL compound or control solutions, 4.5 μL of buffer A (50 mM TRIS-HCl pH 7.4, 2 mM DTT, 0.02% Tween 20, 0.02 mM Na₃VO₄, H₂O nanpure) including a generic concentration of ATP (2 μM f.c.) was added per well, followed by 4.54 of buffer B (4 uM ATP in Buffer A) including a generic concentration of polyEAY (50 nM f.c.), FLT3D835Y kinase, and divalent cations. Final concentration of kinase and cations were: [FLT3D835Y kinase]=5.7 nM, [Mg]=3 mM, [Mn]=3 mM. After 1 hour of incubation the kinase reactions were stopped by the addition of 4.5 μL of stop solution D (50 mM EDTA, 20 mM TRIS-HCl pH 7.4, 0.04% NP-40) immediately followed by 4.5 μL of buffer A (50 mM TRIS-HCl pH 7.4, 2 mM DTT, 0.02% Tween 20, 0.02 mM Na₃VO₄, H₂O nanpure) including the Tb-labeled P-20 antibody to give a total detection volume of 184. After an incubation time of 45 min in the dark, the plates were transferred into the Pherastar fluorescence reader for counting. The effect of compound on the enzymatic activity was obtained from the linear progress curves and determined from one reading (end point measurement). Compounds of the foregoing examples were tested by the FLT3D835Y TR-FRET assay and found to exhibit an IC₅₀ values as shown in TABLE 5 below.

TABLE 5

| EX# | FLT3 IC50 μM | PDGFRa IC50 μM | FGFR3 IC50 μM |
|---|---|---|---|
| 143 | >10 | >10 | >10 |
| 173 | 0.0074 | 0.048 | 0.045 |
| 176 | 1.1 | 8 | 8 |
| 178 | >10 | >10 | >10 |
| 183 | 0.0044 | 0.0230 | 0.0440 |
| 185 | 1.0000 | 4.1000 | >10 |
| 186 | 1.6 | >10 | 3.7 |
| 187 | 0.0017 | 0.0250 | 0.8300 |
| 189 | 0.1700 | 1.7000 | 7.4000 |
| 195 | 0.0035 | 0.033 | 0.011 |
| 196 | >10 | >10 | >10 |
| 197 | 0.0014 | 0.0230 | |
| 199 | 0.0018 | 0.0140 | |
| 210 | 4.2 | 0.4 | >10 |
| 213 | 3.5 | 4 | >10 |
| 219 | 1.7 | 0.63 | >10 |
| 224 | 1.6 | 0.39 | >10 |
| 227 | 0.1400 | 1.3000 | 2.4000 |
| 228 | 0.0027 | 0.0370 | 0.3200 |
| 229 | 0.0930 | 1.0000 | 3.4000 |
| 230 | 0.27 | 1.5 | >10 |
| 232 | 0.83 | 0.63 | >10 |
| 233 | 0.1600 | 0.9400 | |
| 238 | 0.99 | 7.5 | >10 |
| 240 | 1.2000 | 7.3000 | >10 |
| 242 | 1.2 | 0.4 | >10 |
| 243 | 4.1000 | 8.3000 | |
| 286 | | 0.2800 | 0.7100 |
| 291 | 0.0053 | 0.0810 | 1.6000 |
| 292 | 0.5900 | 4.4000 | >10 |
| 296 | 0.0008 | 0.0087 | 0.2 |
| 297 | 0.023 | 0.15 | 1.6 |
| 340 | 0.1100 | 0.8100 | 0.2100 |
| 341 | 0.1200 | 0.8800 | 0.4800 |
| 342 | 0.0120 | 0.1400 | 1.6000 |
| 343 | 0.0018 | 0.0120 | 0.0350 |
| 344 | 0.0100 | 0.1100 | 0.4400 |
| 345 | 0.9200 | 7.4000 | >10 |
| 346 | 0.0085 | 0.0720 | 0.1800 |
| 347 | 0.7700 | 9.0000 | 8.8000 |
| 348 | 0.0170 | 0.1500 | 0.4200 |
| 349 | 0.2500 | 3.3000 | 6.9000 |
| 350 | 0.0440 | 0.4200 | |
| 351 | 0.005 | 0.018 | |
| 352 | 0.0025 | 0.0410 | |
| 353 | 0.0950 | 0.7600 | |
| 354 | 0.0031 | 0.0260 | 0.8700 |
| 355 | 2.6000 | 9.1000 | >10 |
| 356 | 0.0058 | 0.0530 | 0.2200 |

1-12. (canceled)

13. A compound of Formula IA or IB: 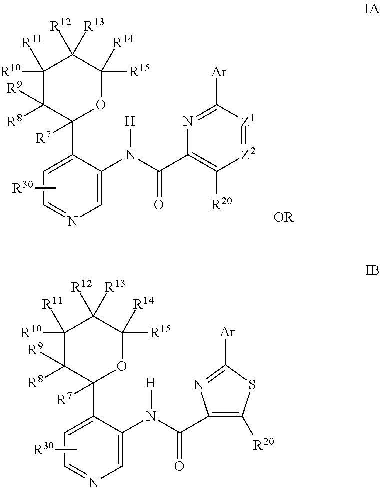
wherein:
Ar is selected from phenyl, pyridyl, pyrazinyl, pyridazinyl, thiazolyl, and pyrazolyl, where Ar is optionally substituted with up to four groups selected from halo, C₁₋₄ alkyl, C₃₋₅ cycloalkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, —(CH₂)₁₋₃—OR, wherein each R is H or C₁₋₄ alkyl or C₃₋₅ cycloalkyl;
Z¹ is N or C—Y, where Y is H, NH₂, F, Cl, or CN;
Z² is CH or N;
R²⁰ is H, D, halo, OH, or NH₂;
R³⁰ is H, D, Me, OMe, CN, or halo;
R⁷ is H, D, Me or CF₃;
R⁸ and R⁹ are independently H, D, Me, OH, NH₂, OMe, or F; or R⁸ and R⁹ taken together represent ═O (oxo):
or R⁷ and R⁸ taken together form a double bond between the carbon atoms to which they are attached;
R¹⁰ and R¹¹ are independently H, D, C₁₋₄ alkyl, C₃₋₅ cycloalkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹⁰ and R¹¹ are linked together to form a 3-6 membered cycloalkyl or heterocycloalkyl ring; or R¹⁰ and R¹¹ taken together represent ═O (oxo) or ═CH₂:
R¹² and R¹³ are independently H, D, C₁₋₄ alkyl, C₃₋₅ cycloalkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹² and R¹³ are linked together to form a 3-6 membered cycloalkyl or heterocycloalkyl ring; or R¹² and R¹³ taken together represent ═O (oxo) or ═CH₂:
R¹⁴ and R¹⁵ are independently H, D, C₁₋₄ alkyl, C₃₋₅ cycloalkyl, C₁₋₄ alkoxy, C₁₋₄ haloalkyl, C₂₋₄ alkenyl, C₂₋₄ alkynyl, —(CH₂)₁₋₃X, OH, NH₂, or F; or R¹⁴ and R¹⁵ are linked together to form a 3-6 membered cycloalkyl or heterocycloalkyl ring;
where each X is independently F, Cl, CN, OH, OMe, or NH₂;
and optionally R¹² can be taken together with either R¹¹ or R¹⁴ to form a 5-6 membered ring containing up to 2 heteroatoms selected from N, O and S as ring members, and optionally substituted with ═O, CN, halo, Me, OMe, OH, or NH₂;
including the tautomers, stereoisomers, and pharmaceutically acceptable salts of these compounds.

14. The compound of claim 13, wherein Z¹ is N, or Z¹ is C—Y, where Y is H, F or CN.

15. The compound of claim 13, wherein R²⁰ is H or NH₂.

16. The compound of claim 13, wherein R³⁰ is H.

17. The compound of claim 13, wherein Ar is unsubstituted phenyl, or Ar is 2-fluorophenyl or 2,6-difluorophenyl that is optionally substituted with one or two additional groups selected from halo, C₁₋₄ alkyl, C₁₋₄ alkoxy, C₁₋₄ haloalkyl, CN, CONR₂, OH, —NRC(O)R, hydroxy-substituted C₁₋₄ alkyl, dihydroxy-substituted C₁₋₄ alkyl, —SO₂R, —SR, or a group of the formula —(CH₂)₁₋₃—OR, or two such groups can be joined together to form a 5-6 membered optionally substituted ring fused to Ar and containing up to two heteroatoms selected from N, O and S as ring members; wherein each R is independently H or C₁₋₄ alkyl, and where two R on the same or adjacent connected atoms can be joined together to form a 5-6 membered ring containing up to two heteroatoms selected from N, O and S as ring members.

18. The compound of claim 17, wherein at least two of R¹⁰, R¹¹, R¹², R¹³, R¹⁴ and R¹⁵ are selected from —OH, NH₂, Me, and Et.

19. The compound of claim 13, which is a compound of Formula IA′ or IB′: 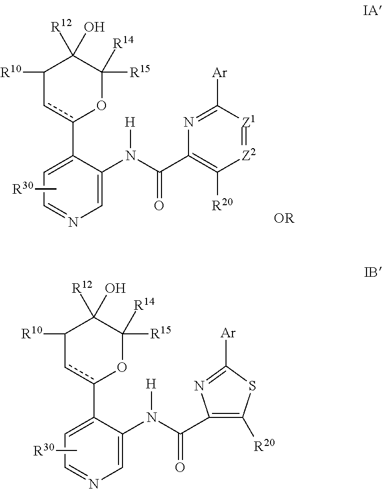
wherein the dashed line represents an optional carbon-carbon double bond;
R¹⁰ is OH or NH₂;
R²⁰ is H or NH₂;
R³⁰ is H;
R¹² is H, Me, Et, or Propyl;
R¹⁴ is selected from H, Me, Et, vinyl, propyl, isopropyl, t-butyl, cyclopropyl and —(CH₂)₁₋₃—X, where X is OH, CN, OMe, or halo, and R¹⁵ is H or Me;
or R¹⁴ and R¹⁵ taken together form a spirocyclopropane ring.

20. The compound of claim 19, which is of the formula:

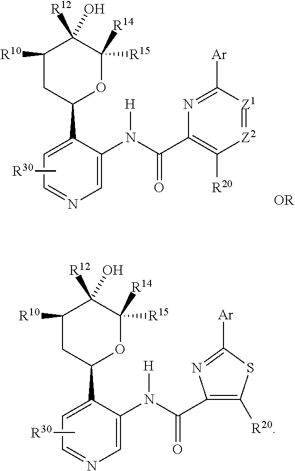

21. A compound of Formula II, or a pharmaceutically acceptable salt thereof, 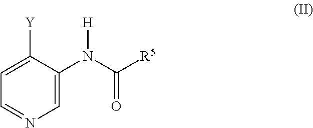
wherein,
Y is selected from tetrahydropyran, dioxane, dihydro-2H-pyran, dioxolane, dihydro-2H-pyran-4-(3H)-one, 5-methylenetetrahydro-2H-pyran-4-ol, 3,4-dihydro-2H-pyran-4-ol, 2H-pyran-4(3H)-one, and tetrahydrofuran, wherein each said Y group is independently substituted with at least one of R⁷, R⁸, R⁹, R¹⁰, R¹¹, R¹², R¹³, R¹⁴, and R¹⁵;
R⁵ is selected from a group consisting of thiazole, pyridine, pyrimidine, triazine, and pyrazine, wherein each said R⁵ group is substituted with one to three substituents selected from R¹⁸, R¹⁹, and R²⁰;
R⁷ is selected from C₁₋₄-alkyl, H, D, F, and C₁₋₄-halo alkyl;
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group, or C₃₋₈-heterocycloalkyl group;
R¹⁸, R¹⁹, and R²⁰ independently are selected from H, aryl, pyridine, thiazole, pyrimidine, pyrazine, pyridazine, amino, C₃₋₈-cycloalkyl or a C₃₋₈-heterocycloalkyl, cyano, halogen, and C₁₋₄-alkyl, wherein said aryl, pyridine, thiazole, pyrimidine, pyrazine, pyridazine, amino and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, oxo, —SO₂—C₁₋₄ alkyl, CO—NH—C₃₋₆-branched alkyl, OC₁₋₄-alkyl, and OC₁₋₄-haloalkyl.

22. The compound of claim 21, wherein:
Y represents tetrahydropyran, or dihydro-pyran, wherein each said Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

23. The compound of claim 21, wherein Y represents tetrahydropyran.

24. The compound of claim 21, wherein Y represents dihydro-pyran.

25. The compound of claim 21, wherein:
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, oxo, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl₁ group.

26. The compound of claim 21, wherein:
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₆ cycloalkyl, C₃₋₆ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

27. The compound of claim 21, wherein:
Y represents tetrahydrofuran, or dihydro-2H-pyran-4(3H)-one, wherein each Y group is substituted with at least one of R⁷, R⁸, R⁹, R¹⁰, R¹¹, R¹², R¹³, R¹⁴, and R¹⁵;
R⁷ is selected from methyl, H, D, and trifluoro-methyl; and
R⁸, R⁹, R¹⁰, R¹¹, R¹², R¹³, R¹⁴, and R¹⁵ independently at each occurrence are selected from H, hydroxy, D, hydroxy-methyl, Cl, chloro-methyl, F, methyl, ethyl, amino, ethylene, cyano, hydroxymethyl, fluoromethyl, difluoromethyl, trifluoromethyl, vinyl, acetylene, and cyano-methyl; alternatively any two of R⁸, R⁹, R¹⁰, R¹¹, R¹², R¹³, R¹⁴, and R¹⁵ along with the carbon atom to which they are attached can be taken together to form a C₃₋₈-cycloalkyl group or C₃₋₈-heterocycloalkyl group.

28. The compound of claim 21, wherein:
R⁵ is selected from a group consisting of thiazole, pyridine, pyrimidine, triazine and pyrazine, wherein each said R⁵ group is substituted with one to three substituents selected from R¹⁸, R¹⁹, and R²⁰;
R¹⁸, R¹⁹, and R²⁰ independently are selected from H, phenyl, pyridine, thiazole, pyrimidine, pyridazine, pyrazine, amino, cyano, halogen, C₃₋₈ cycloalkyl, C₃₋₈ heterocycloalkyl, and C₁₋₄-alkyl, wherein said aryl, heteroaryl and alkyl groups are further substituted with at least one of R²¹, R²², and R²³; and
R²¹, R²², and R²³ independently are selected from halogen, C₁₋₄-alkyl, hydroxy, amino, CN, NO₂, H, COOH, CONH—C₁₋₄ alkyl, CO—NH—C₃₋₆-branched alkyl, OC₁₋₄-alkyl, and OC₁₋₄-haloalkyl.

29. The compound of claim 21, which is selected from the compounds 1-356 in Table 1.

30. A pharmaceutical composition comprising a compound of claim 13 admixed with at least one pharmaceutically acceptable excipient.

31. The pharmaceutical composition of claim 30, wherein said pharmaceutical composition comprises an additional agent for the treatment of cancer.

32. The pharmaceutical composition of claim 31 wherein the additional agent is selected from irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib (Gleevec), anthracyclines, rituximab, and trastuzumab.

33. A method for treating a condition by modulation of Provirus Integration of Maloney Kinase (PIM Kinase), GSK3, PKC, KDR, PDGFRa, FGFR3, FLT3, or cABL activity comprising administering to a patient in need of such treatment an effective amount of a compound of claim 13.

34. The method of claim 33, wherein the condition is selected from carcinoma of the lungs, pancreas, thyroid, ovarian, bladder, breast, prostate, or colon, melanoma, myeloid leukemia, multiple myeloma and erythro leukemia, villous colon adenoma, and osteosarcoma.

35. The method of claim 33, wherein the condition is an autoimmune disorder selected from Crohn's disease, inflammatory bowel disease, rheumatoid arthritis, and chronic inflammatory diseases.

36-38. (canceled)
